(19) United States
(12) Patent Application Publication
(10) Pub. No.: US 2006/0188885 A1
(43) Pub. Date: Aug. 24, 2006

(54) High throughput functional genomic screening methods for osteoarthritis

(76) Inventors: Dale Lesley Bodian, Whippany, NJ (US); Sherif Daouti, Hasbrouck Heights, NJ (US); Chandrika Saidapet Kumar, Acton, MA (US); Brian Jude Latario, Groton, MA (US); Joseph Quintavalla, New Providence, NJ (US)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/553,520

(22) PCT Filed: Apr. 16, 2004
(86) PCT No.: PCT/EP04/04055

§ 371 Date: Oct. 14, 2005

Related U.S. Application Data
(60) Provisional application No. 60/463,933, filed on Apr. 18, 2003.

(51) Int. Cl. C12Q 1/68

(52) U.S. Cl. 435/6

(57) ABSTRACT

High-throughput functional screening assays are provided that identify genes and gene products that are associated with the pathogenesis of osteoarthritis (OA) in chondrocytes. In addition, genes and gene products identified by such functional assays are also provided. The genes and gene products provided herein are useful inter alia for diagnosing OA in individuals and as drug targets for identifying drugs to treat OA.

### FIELD OF THE INVENTION

The present invention provides novel functional genomic screening methods for identifying genes and gene products that are involved in OA. Genes and gene products are also provided that have been identified in such screening assays and which are useful inter alia as drug targets for treating OA. Methods of treating and diagnosing OA and compositions therefor which use genes and/or gene products identified in these screening assays are also provided.

### BACKGROUND

Osteoarthritis (OA) is primarily a non-inflammatory disease characterized by pain and stiffness of the joints caused by the progressive loss of articular cartilage. OA is among the most common age associated disease and is estimated to affect about 56 million individuals worldwide or 80% of the population greater than 60 years old. Although OA is generally considered a degenerative disorder, the disease is associated with activation of chondrocyte cells, the major cell type present in normal articular cartilage. Hallmarks of this cell activation include hypertrophy, proliferation, dedifferentiation, degradation of the existing extracellular matrix, and finally apoptosis.

The molecular etiology of OA remains unknown. Current therapeutic methods for treating OA are therefore directed toward symptomatic relief such as reducing joint pain and secondary inflammatory changes rather than toward treating the disease's underlying causes. Pharmacological interventions that prevent disease progression are not currently available. Many patients thus progress to advanced stages of the disease where total joint replacement surgery is necessary. For reviews, see Pritzken, “Pathology of Osteoarthritis” in Osteoarthritis (Brandt et al., Eds.) Oxford University Press 1998, pages 50-61. See also, Sandell & Aigner, Arthritis and Rheumatism 2001, 3:107-113.

Large scale sequencing of OA cDNA libraries has identified several putative gene products that are expressed by diseased chondrocyte cells. See, Stokes et al., Arth. Rheum. 2002, 46:404-419; Hu et al., J. Biol. Chem. 1998, 51:34406-34412; Aigner et al., Arth. Rheum. 2001, 44:2777-2789. However, functional information is not presently available for these gene products and their role in OA, if any, remains unknown. The molecular basis of OA therefore remains unknown and only a very limited number of potential drug targets is known. There remains a need, therefore, for therapeutic compounds and methods to treat OA and related diseases. There is moreover a need for novel genes and gene products that may be useful, e.g., as drug targets for such therapeutic methods to treat OA.

In order to identify genes associated with OA that can serve as suitable drug targets, Applicants disclose herein several high throughput screening methods that may be used successfully with chondrocytes. Identification of genes that are critical in mediating the diseased phenotype requires development of comprehensive highly sensitive cell-based assays compatible with high-throughput settings. The availability of methods to shuttle full length cDNA clones from one vector into another (Gateway system, Invitrogen, Carlsbad, Calif.) combined with the ability to express genes in high levels in disease relevant primary cells using viral vectors and the availability of methods for assay miniaturization and liquid handling have lead to the possibility of efficiently screening for inducers of OA phenotype on a genome wide scale.

Using said methods, Applicants have identified several genes (referred to herein as “candidate genes”) in chondrocytes that are associated with OA. Thus, according to the present invention, it is now proposed that these genes and gene products have a role in OA pathogenesis and it is contemplated herein that any one or more of them are useful drug targets for the development of therapeutics for the prevention, treatment or amelioration of OA or related conditions associated with abnormal cartilage degradation.

The invention also provides a method for identifying modulators (e.g. inhibitors) of these newly identified OA related genes and the use of such modulators for the treatment, prevention, or amelioration of this disease and related conditions, in human and veterinary patients. The invention also provides pharmaceutical compositions comprising said modulators.

### SUMMARY OF THE INVENTION

The present invention provides high throughput functional genomic screening (HTS) assays that may be used to identify genes and gene products associated with OA. In preferred embodiments, a HTS assay of the invention comprises steps of transfecting a cell (preferably a chondrocyte cell) with a nucleic acid to be tested in the screening assay (i.e., a “test” nucleic acid) so that the test nucleic acid is expressed by the cell. The transfected cell is then assayed for one or more characteristics that are associated with OA. For example, in one preferred embodiment, a screening assay of the invention comprises steps of detecting expression by the cell of one or more genes or gene products whose expression is known to be associated with OA.

Similarly, screening assays of the invention can be used to identify polypeptides and other gene products that are associated with OA in cells. Such methods involve transfecting a cell preferably a chondrocyte cell) with a nucleic acid that encodes a polypeptide or other gene product to be tested in the screening assay (Le., a “test” polypeptide) so that the test polypeptide is expressed by the cell. The transfected cell is then assayed for one or more characteristics that are associated with OA. For example, in one preferred embodiment a screening assay of the invention comprises steps of detecting expression by the cell of one or more genes or gene products whose expression is known to be associated with OA.

A variety of known genes and gene products associated with OA are provided in the application and can be used in the above-described assays. Preferred genes and gene products that are associated with OA ( or an “OA phenotype” include, for example, an Aggrecanase-1 gene, an MMP-13 gene, genes of Collagen Types I, IIa and X, an iNOS gene, an Aggrecan gene or gene product, and a Decorin gene, as well as gene products encoded by any of these genes. Still other genes or gene products that are associated with an OA phenotype and can be used in the methods described here include new marker genes C17, SMOC2, OSF-2, MARCKS, retinoic acid receptor beta, Zic1, BASP1 and DIM1 genes and their gene products which were identified by computational analysis of OA cDNA libraries.

In another aspect, the Applicants have discovered that genes and gene products for an OA phenotype may be rapidly screened by identifying gene and gene products that induce the proliferation of chondrocyte cells. Hence, the invention also provides, in another aspect, a method for identifying a nucleic acid that induces an OA phenotype by transfecting a chondrocyte cell with a candidate nucleic acid, and detecting proliferation of the chondrocyte cell (e.g., by identifying clusters of clonally proliferating chondrocyte cells in cell culture). Similarly, the invention provides methods for identifying a polypeptide that induces an OA phenotype in cells, by transfecting a chondrocyte cell with a nucleic acid that encodes a candidate polypeptide, and detecting proliferation of the chondrocyte cell (e.g., by identifying clusters of clonally proliferating chondrocyte cells in cell culture). In such methods, proliferation of the chondrocyte cells indicates that the candidate nucleic acid or polypeptide is a nucleic acid or polypeptide that induces an OA phenotype.

Genes and gene products that are identified by such screening methods are useful, inter alia, for the diagnosis and treatment, prevention and/or amelioration of OA. For example, candidate genes and gene products identified by these screening methods may be used in still other screening assays, to identify compounds that bind to and/or inhibit expression of these candidate genes and gene products. The compounds (i.e., modulators) identified in these screening assays are useful, e.g., in therapeutic methods for treating OA and as pharmaceutical compositions or medicaments that can be administered in such therapeutic methods. Thus the present invention also pertains to the use of these genes, gene products, compounds and modulators in the manufacture of a medicament and/or as a pharmaceutical for the treatment, prevention and/or amelioration of OA and other cartilage-related diseases.

In still other embodiments, the invention provides methods for treating, preventing and/or ameliorating OA in an individual, by administering an effective amount of a compound that can modulate (i.e. a “modulator”) a candidate gene identified by the assay and methods of the present invention. In a preferred embodiment, the modulator inhibits a candidate gene disclosed in Tables V or VI disclosed herein. The invention also provides pharmaceutical compositions that comprise an effective amount of a modulator to a candidate gene identified herein.

Thus, in another aspect, the invention relates to a method to treat, prevent or ameliorate OA, comprising administering to a subject in need thereof a pharmaceutical composition comprising an effective amount of a modulator of a candidate gene and/or ligand thereo (i.e a gene provided in Tables V or VI provided herein. In various preferred embodiments, said pharmaceutical composition comprises one or more modulators to any one or more of said candidate genes and/or ligands thereof.

In another aspect, the invention relates to a pharmaceutical composition comprising a modulator of a candidate gene and/or ligand thereof in an amount effective to treat, prevent or ameliorate OA in a subject in need thereof wherein said modulator, e.g., can inhibit the activity, expression of or ligand binding to, any one or more of the candidate genes disclosed herein e.g., a candidate gene provided in Tables V or VI herein. In one embodiment, said pharmaceutical composition comprises any one or more substances selected from the group consisting of antisense oligonucleotides, triple helix DNA, siRNA, ribozymes, RNA aptamers or double or single stranded RNA directed to a nucleic acid sequence of a candidate gene or ligand thereof wherein said substances are designed to inhibit gene expression of said family member or ligand. In a further embodiment, said pharmaceutical composition comprises antibodies to a candidate gene or ligand thereof, or fragments thereof, wherein said antibodies can, e.g., inhibit the activity of said member and/or ligand.

In yet another aspect of the present invention there are provided assay methods and kits comprising the components necessary to detect expression of polynucleotides encoding a candidate gene or ligand thereof, or polypeptide levels of said candidate genes or ligands thereof, or fragments thereof, in biological samples derived from a patient, such kits comprising, e.g., antibodies that bind to said polypeptides, or to fragments thereof, or oligonucleotide probes that hybridize with said polynucleotides. In a preferred embodiment, such kits also comprise instructions detailing the procedures by which the kit components are to be used.

The present invention also provides methods for identifying individuals who have OA. Such diagnostic methods involve detecting a candidate gene or gene product (identified by one of the high throughput functional assays described, supra) in a biological sample (e.g., chondrocyte cell or cartilage tissue sample) from the individual. Elevated expression of the candidate gene or gene product in the chondrocyte cell or cartilage tissue indicates that the individual does have OA.

The invention also provides methods for identifying compounds that may be used to treat OA. In a first embodiments, these methods involve contacting a test compound to a candidate gene or gene product under conditions sufficient to allow the test compound to bind to a candidate gene or gene product of the invention, and detecting complexes of the test compound bound to that candidate gene or gene product. The detection of the test compound bound to the candidate gene or gene product identifies the test compound as a compound that can be used for treating OA.

In another embodiment, methods for identifying compounds that may be used to treat OA involve contacting a test compound to a cell that normally expresses a candidate gene or gene product of the invention, and detecting expression of that candidate gene or gene product by the cell once it has been contacted with the test compound. In such embodiments, a decreased expression of the candidate gene or gene product by the cell in the presence of the test compound indicates that the test compound is a compound that can be used to treat OA.

### DETAILED DESCRIPTION

As used herein and in the appended claims, the singular forms “a”, “an”, and “the” include plural reference unless the context clearly dictates otherwise. Thus, for example, reference to the “antibody” is a reference to one or more antibodies and equivalents thereof known to those skilled in the art, and so forth.

“Nucleic acid sequence”, as used herein, refers to an oligonucleotide, nucleotide or polynucleotide, and fragments or portions thereof, and to DNA or RNA of genomic or synthetic origin that may be single or double stranded, and represent the sense or antisense strand.

As used herein, “high throughput” refers to an increase in screening capacity compared to conventional methods. It is contemplated herein that the high throughput method of the present invention is preferably carried out using microtiter plates (i.e. 96, 384 or 1536 well plates). Assays at a genomic level are also contemplated.

cDNA libraries for use with the high throughput screen disclosed herein are those wherein each cDNA is defined and arrayed in a specific order in high throughput format (multititer dishes). While the examples in the present invention describe results obtained with a proprietary cDNA collection, suitable cDNA libraries are commercially available, for example, from Invitrogen (Carlsbad, Calif.), Origene (Rockville, Md.) as well as the NIH (i.e., the Mammalian Gene Collection).

The term “antisense” as used herein, refers to nucleotide sequences which are complementary to a specific DNA or RNA sequence. The term “antisense strand” is used in reference to a nucleic acid strand that is complementary to the “sense” strand. Antisense molecules may be produced by any method, including synthesis by ligating the gene(s) of interest in a reverse orientation to a viral promoter which permits the synthesis of a complementary strand. Once introduced into a cell, this transcribed strand combines natural sequences produced by the cell to form duplexes. These duplexes then block either the further transcription or translation. The designation “negative” is sometimes used in reference to the antisense strand, and “positive” is sometimes used in reference to the sense strand.

“cDNA” refers to DNA that is complementary to a portion of messenger RNA (mRNA) sequence and is generally synthesized from an mRNA preparation using reverse transcriptase.

As contemplated herein, antisense oligonucleotides, triple helix DNA, RNA aptamers, ribozymes, siRNA and double stranded RNA are directed to a nucleic acid sequence such that the nucleotide sequence chosen will produce gene-specific inhibition of gene expression. For example, knowledge of a nucleotide sequence may be used to design an antisense molecule which gives strongest hybridization to the mRNA. Similarly, ribozymes can be synthesized to recognize specific nucleotide sequences of a gene and cleave it (Cech. J. Amer. Med Assn. 260:3030 (1988)). Techniques for the design of such molecules for use in targeted inhibition of gene expression is well known to one of skill in the art.

The individual candidate gene products, (i.e. proteins/polypeptides) referred to herein include any and all forms of these proteins including, but not limited to, partial forms, isoforms, variants, precursor forms, the full length protein, fusion proteins containing the sequence or fragments of any of the above, from human or any other species. Protein homologs which would be apparent to one of skill in the art are included in this definition. It is also contemplated that the term refers to proteins isolated from naturally occurring sources of any species such as genomic DNA libraries as well as genetically engineered host cells comprising expression systems, or produced by chemical synthesis using, for instance, automated peptide synthesizers or a combination of such methods. Means for isolating and preparing such polypeptides are well understood in the art.

The terms “sample” or “biological sample” as used herein, are used in their broadest sense. A biological sample from a subject may comprise blood, urine or other biological material with which protein activity or gene expression may be assayed. A biological sample may include, for example, cells, cartilage, blood, tumors or other specimens from which total RNA may be purified for gene expression profiling using, for example, conventional glass chip microarray technologies such as Affymetrix chips, RT-PCR or other conventional methods.

As used herein, the term “antibody” refers to intact molecules as well as fragments thereof such as Fa, F(ab′)₂, and Fv, which are capable of binding the epitopic determinant. Antibodies that bind specific polypeptides can be prepared using intact polypeptides or fragments containing small peptides of interest as the immunizing antigen. The polypeptides or peptides used to immunize an animal can be derived from the translation of RNA or synthesized chemically, and can be conjugated to a carrier protein, if desired. Commonly used carriers that are chemically coupled to peptides include bovine serum albumin and thyroglobulin. The coupled peptide is then used to immunize an animal (e.g., a mouse, a rat or a rabbit).

The term “humanized antibody” as used herein, refers to antibody molecules in which amino acids have been replaced in the non-antigen binding regions in order to more closely resemble a human antibody, while still retaining the original binding ability.

A “therapeutically effective amount” is the amount of drug sufficient to treat, prevent or ameliorate pathological conditions associated with OA.

“Subject” or “individual” refer to any human or nonhuman organism.

The high throughput assay disclosed herein is preferably used or performed in an at least substantially automated setting. A multiwell format is suited for performing at least part of the methods of the present invention, but can be performed on many different scales, including screening cDNAs on a genomic scale. The term “automated” as used herein means able to perform the predetermined steps of the method without, for the most part, requiring manual intervention during the process. In this regard, machines for use in the high throughput methods disclosed herein include, but are not limited to, machines for preparing DNA plasmid preparations, reading DNA concentration and yield, plating cells, automated pipeting stations and luminescence detectors. Such machines are commercially available and familiar to one of skill in the art, for example, the Quiagen 8000 for automated DNA production (Qiagen Inc, Valencia Calif.), the Beckman Coulter BiomekFX for automated pipetting and transfections (Beckman Coulter, Fullerton Calif.) and the Fluoroskan Ascent for fluorescent and luminescent assay readouts (Thermo Labsystems, Franklin, Mass.).

Nucleic acid transfer into cells (e.g. transfection) may be performed according to any conventional method familiar to one of skill in the art. As mentioned above, transfections are preferably implemented in an automated, multiwell, high throughput format, for example, using commercially available robotics such as a Beckman Coulter BiomekFX.

The present invention provides high throughput screening (ITS) assays that are useful, inter alia for identifying therapeutic agents to treat and/or diagnose disorders such as osteoarthritis (OA) that affect the growth and/or degradation of-cartilage. In particular, the Examples infra describe particular, preferred embodiments of screening assays that identify genes and gene products associated with OA. The genes and gene products identified in such screening assays are therefore useful, e.g., as drug target candidates for the development of novel drug therapies to treat OA and other such cartilage disorders. For convenience therefore, the genes and gene products identified in screening assays of the present invention are generally referred to in this document as “candidate” genes and “candidate” gene products, respectively.

Generally speaking, the HTS assays of this invention allow a user to rapidly screen large numbers of genes, e.g., in a cDNA library, to identify ones that are involved in OA. Briefly, nucleic acids (preferably cDNA molecules) corresponding to the genes to be tested in a screening assay are first transferred to expression vectors that are capable of expressing those “test” genes or gene products in chondrocyte cells. Preferred expression vectors are retroviral vectors (such as those described in the Examples, infra) or other vectors that are capable of expressing the candidate genes at high levels in chondrocyte cells.

Chondrocyte cells are then transformed with the expression vectors carrying these test genes and are assayed for one or more characteristics that are associated with OA. For convenience, such characteristics are generally referred to in this application as “OA phenotypes.” However, it is understood that a characteristic assayed or tested for in these screening assays may be any feature that is associated with OA.

For instance, Example 1 describes one preferred embodiment of a HTS assay that uses RT-PCR to measure the expression of one or more genes whose expression in chondrocyte cells is associated with OA. Examples of such genes which are preferred in these methods include Aggrecanase-1 and MMP-13 (the expression of which is associated with cartilage degradation), Collagen Type I, Collagen Type IIa and Collagen Type X (the over expression of which is associated with aberrant chondrocyte cell differentiation such as hypertrophy and proliferation), genes and gene products that induce inflammation (for example, iNOS and Cox-2), and genes such as Aggrecan and Decorin that modulate synthesis or repair of the cartilage matrix.

Such genes, whose expression or, more particular, over expression is indicative of OA in chondrocyte cells, are generally referred to here as “marker genes.” However, “marker genes” that may be used in screening assays of the invention are not limited to the particular genes described, e.g., in the examples (see, for example, in Table I or Table II, infra). Any gene or gene product whose elevated expression in chondrocyte cells is associated with OA may be used as a marker gene in screening assays according to the present invention. For example, and as explained in further detail below, the screening assays of this invention identify other genes and gene products whose elevated expression is associated with OA. Hence, a candidate gene or gene product identified in such screening assays (for example, any of the candidate genes and gene products listed in Tables V and VI infra) may itself be used as a marker gene in another screening assay according to this invention.

Similarly, those who are skilled in the art will appreciate that marker genes which can be used in screening assays of this invention are not limited to gene whose over expression is associated with OA. In particular, a screening assay of the present invention can also use marker genes that are underexpressed (i.e., their expression is reduced) in OA chondrocytes. In such embodiments, the HTS assays of this invention will identify candidate genes that, when expressed in chondrocyte cells, cause the reduced expression of one or more marker genes.

The HTS assays of this invention also are not limited to embodiments that measure the expression of marker genes or their gene products. Other characteristics or phenotypes associated with OA can also be measured or observed, and then used to identify candidate genes in a screening assay. For instance, Example 2 infra describes an alternative embodiment of the screening assay which identify cDNAs that induce a particular type of cell proliferation characteristic of OA chondrocytes. In particular, whereas normal chondrocyte cells have a low division rate when grown in a 3-dimensional matrix (e.g., of agarose or alginate), OA chondrocyte cells (both in cell culture and in OA cartilage tissue) grow in clusters of rapidly proliferating chondrocyte cell clones. Accordingly, screening assays of the invention can also identify genes and gene products which, when expressed in chondrocyte cell cultures, cause the formation of such clusters of chondrocyte cell clones.

Genes and gene products that are tested in a screening assay of the invention may be from any source and obtained by any method known in the art. For example, cDNA libraries may be derived from a cell or cell line of interest, which is preferably a chondrocyte cell. Methods for obtaining such cDNA libraries are well known in the art. See, for example, Sambrook et al., Molecular Cloning: A Laboratory Manual, Second Edition (1989) Cold Spring Harbor Laboratory Press, Cold Spring Harbor, N.Y.); Glover, D. M. 3ed., 1985, DNA Cloning: A Practical Approach, MRL Press, Ltd. Oxford U.K. Vols. I and II). See also, in the Examples, infra. Alternatively, however, the genes and Gene Products may be hand selected. For instance, Example 1 describes an embodiment where the genes in a cDNA library are first “datamined” to identify genes and gene products that are particularly useful as drug targets (e.g., for therapeutic compounds to treat OA). Examples of such preferred test genes are genes that are involved in signal transduction and/or proteolysis (such as receptors, kinases and proteases).

Candidate genes and gene products that are identified in screening assays of the present invention are useful, inter alia, as new marker genes for identifying osteoarritic cells (i,e., cells that are present in cartilage from patients having OA and/or which exhibit one or more characteristics associated with OA). Moreover, the genes and gene products identified in these screening assays can also be used in diagnostic and prognostic applications. Hence, the candidate genes and gene products that are identified in the screening assays provided here can be used to identify individuals who have a disorder, such as OA, that is associated with abnormal cartilage growth and/or repair.

The candidate genes and gene products identified in screening assays of this invention can also be used in prognostic applications to identify individuals who are either have OA or who are at an increased risk of developing OA. Hence, the invention also provides therapeutic methods for treating OA related disorders in individuals. Such methods involve administering a compound to an individual that inhibits the expression or activity of a candidate gene identified in a screening assay of the invention or, alternatively, a compound that inhibits the expression or activity of a candidate gene product identified in a screening assay of this invention.

Various applications and uses for candidate genes and gene products identified in the present invention are described, in detail infra. In particular, the following sections first describe various homologs and analogs of both candidate genes and candidate genes products that can be used in such prognostic, diagnostic, and therapeutic assays. Particular utilities for these candidate genes and gene products (including the various homologs and analogs thereof) are then also described in detail. Finally, the Examples describe detailed, exemplary embodiments of screening assays that are considered part of the present invention. These examples also provide Tables identifying the nucleotide and amino acid sequence (by GenBank Accession number) of both genes and gene products that are identified in such screening assays. These nucleotide and amino acid sequences are therefore considered examples of preferred embodiments of candidate genes and gene products of the invention.

The present invention may employ a variety of conventional techniques in the arts of molecular biology, microbiology and recombinant DNA technology. Such techniques are well known in the art and are explained fully in the literature. See, for example, Sambrook, Fitsch & Maniatis, Molecular Cloning: A Laboratory Manual, Second Edition (1989) Cold Spring Harbor Laboratory Press, Cold Spring Harbor, NY (referred to herein as “Sambrook et al., 1989); DNA Cloning: A Practical Approach Volumes I and II (D. N. Glover et al. 1985); Oligonucleotide Synthesis (M. J. Gait ed. 1984); Nucleic Acid Hybridization (B. D. Hames & S. J. Higgins, eds. 1984); Animal Cell Culture (R. I. Freshney, ed. 1986); Immobilized Cells and Enzymes (IRL Press, 1986); B. E. Perbal, A Practical Guide to Molecular Cloning (1984); F. M. Ausubel et al. (eds.), Current Protocols in Molecular Biology, John Wiley & Sons, Inc.

Candidate Polypeptides:

It is understood that, as used in the description of this invention, the term “candidate polypeptide” refers to the polypeptide encoded by a candidate gene of the invention. For convenience, candidate genes and gene products of the present invention are frequently identified here by SEQ ID number and by the GenBank Accession Number(s) for preferred nucleotide or amino acid sequences. However, it is understood that the candidate genes and gene products of this invention are not limited to these particular sequences, but also include homologs and variants evident to one of ordinary skill in the art.

As an example, and not by way of limitation, candidate gene product polypeptides of the present invention include not only polypeptides having the exemplary full length amino acid sequences specified here, but also include polypeptides comprising an amino acid sequence for one or more epitopes or domains of a full length candidate gene product polypeptide. An epitope of a polypeptide represents a site on the polypeptide against which an antibody may be produced and to which the antibody binds. Therefore, polypeptides comprising the amino acid sequence of a candidate gene product epitope are useful for making antibodies to the candidate polypeptide. Preferably, an epitope comprises a sequence of at least 5, more preferably at least 10, 15, 20, 25 or 50 amino acid residues in length. Thus, polypeptides of the invention that comprises epitopes of a candidate gene product preferably contain an amino acid sequence corresponding to at least 5, at least 10, at least 15, at least 20, at least 25 or at least 50 amino acid residues of a full length candidate gene product polypeptide sequence.

Candidate gene products of the invention also include analogs and derivatives of the exemplary full length candidate gene product sequences provided in the Examples, infra. Analogs and derivatives of the candidate gene products of this invention have the same or homologous characteristics of the exemplary candidate gene product sequences set forth in the Examples, infra. Chimeric or fusion polypeptides can also be prepared in which the candidate gene product portion of the fusion polypeptide has one or more characteristics of the candidate gene product. Such fusion polypeptides therefore represent embodiments of the candidate gene product polypeptides of this invention. Such fusion polypeptides may also comprise the amino acid sequence of a marker polypeptide; for example FLAG, a histidine tag, glutathione S-transferase (GST), or the Fc portion of an IgG to name a few. Additionally, fusion polypeptides of the invention may comprise amino acid sequences that increase solubility of the polypeptide, such as a thioreductase amino acid sequence or the sequence of one or more immunoglobulin proteins (e.g., IgG1 or IgG2).

Analogs or variants of a candidate polypeptide can also be made by altering encoding nucleic acid molecules, for example by substitutions, additions or deletions. Preferred analogs or variants of a candidate polypeptide are “function conservative variants” of the particular candidate polypeptide sequence specified in the Examples, infra. “Function-conservative variants” of a polypeptide or polynucleotide are those in which a given amino acid residue in the polypeptide, or the amino acid residue encoded by a codon of the polynucleotide, has been changed or altered without altering the overall conformation and function of the polypeptide. Such changes are expected to have little or no effect on the apparent molecular weight or isoelectric point of the polypeptide. Hence, such altered-nucleic acid molecules preferably encode functionally similar molecules (i.e., molecules that perform one or more functions of a candidate polypeptide and/or have one or more of the candidate polypeptide's bioactivities).

Amino acid residues, other than ones that are specifically identified herein as being conserved, may differ among variants of a protein or polypeptide. Accordingly, the percentage of protein or amino acid sequence similarity between any two variants or analogs of a candidate polypeptide may vary. Typically, the percentage of protein or amino acid sequence similarity between variant or analog candidate polypeptides may be from 70% to 99%, as determined according to an alignment scheme such as the Cluster Method and/or the MEGALIGN or GCG alignment algorithm. Preferred variants and analogs of a candidate polypeptide are at least about 75%, and more preferably at least about 80%, 85%, 90%, 95% or 99% sequence identity as determined by a sequence comparison algorithm such as BLAST, FASTA, DNA Strider, CLUSTAL, etc.

Function-conservative variants of the present invention, as defined above, include not only variants of the full length candidate polypeptides of this invention (e.g., variants of polypeptides comprising the particular candidate polypeptide sequences specified in the Examples, infra), but also include function-conservative variants of modified candidate polypeptides (e.g., truncations and deletions) and of fragments (e.g., corresponding to domains or epitopes) of full length candidate polypeptides.

In yet other embodiments, an analog of a candidate polypeptide is an allelic variant or mutant of a candidate polypeptide sequence provided, e.g., in the Examples, infra. The terms allelic variant and mutant, when used herein to describe a polypeptide, refer to a polypeptide encoded by an allelic variant or mutant gene. Thus, the allelic variant and mutant candidate polypeptides of this invention are polypeptides encoded by allelic variants or mutants of a candidate nucleic acid of the present invention.

In yet other embodiments, an analog of a candidate polypeptide is a substantially homologous polypeptide from the same species (e.g., allelic variants) or from another species (e.g., an orthologous polypeptide). The term “homologous,” in all its grammatical forms and spelling variations, refers to the relationship between two proteins or nucleic acids that possess a “common evolutionary origin”, including proteins from superfamilies (e.g., the immunoglobulin superfamily) in the same species of organism as well as homologous proteins from different species of organism (for example, myosin light chain polypeptide, etc.; see, Reeck et al., Cell 1987, 50:667). Such proteins (and their encoding nucleic acids) having sequence homology, as reflected by their sequence similarity, whether in terms of percent identity or by the presence of specific residues or motifs and conserved positions. Preferred homologous polypeptides of the present invention have levels of sequence similarity or identity as specified, above, for other variant and analog candidate polypeptides of the invention. Homologs and orthologs of the specific candidate polypeptides may be obtained, e.g., from mammals such as humans, mice, rats, hamsters, rabbit, guinea pig, dog, cat, sheep, goat, pig, horse and cow to name a few.

In other embodiments, variants of a candidate polypeptide (including analogs, homologs, etc.) are polypeptides encoded by nucleic acid molecules that hybridize to the complement of a nucleic acid molecule encoding one or more of the particular candidate polypeptide sequences specified in the Examples, infra. A nucleic acid molecule is “hybridizable” to another nucleic acid molecule (for example cDNA, genomic DNA, or RNA) when a single stranded form of the nucleic acid molecule can anneal to the other nucleic acid molecule under appropriate conditions of temperature and solution ionic strength (see, e.g., Sambrook et al., supra). The conditions of temperature and ionic strength determine the “stringency” of the hybridization. For preliminary screening for homologous nucleic acids, low stringency hybridization conditions corresponding to a melting temperature (Tₘ) of about 55° C. can be used (for example, 5×SSC, 0.1% SDS, 0.25% milk and no formamide; or, alternatively, 30% formamide, 5×SSC, and 0.5% SDS). Moderate stringency hybridization conditions correspond to a higher Tₘ., e.g., 40% formamide with 5× or 6×SSC. High stringency hybridization conditions correspond to the highest Tₘ, e.g., 50% formamdie, 5× or 6×SSC. A 1×SSC solution is understood to be a solution containing 0.15 M NaCl and 0.015 M Na-citrate.

Hybridization requires that the two nucleic acids contain complementary sequences, although depending on the stringency of the hybridization, mismatches between bases are possible. The appropriate stringency for hybridizing nucleic acids depends on the length of the nucleic acids and the degree of complementation, variables well known in the art. The greater the degree of similarity or homology between two nucleotide sequences the greater the value of Tₘ for hybrids of nucleic acids having those sequences.

For hybrids of greater than 100 nucleotides in length, equations for calculating Tₘ have been derived (see, Sambrook et al., supra, 9.50-9.51).

In a specific embodiment, the term “standard hybridization conditions” refers to a Tₘ of about 55° C. and utilizes conditions as set forth above. In a preferred embodiment, the Tₘ is 60° C.; in a more preferred embodiment, the Tₘ is 65° C. In a specific embodiment, the term “high stringency” refers to hybridization and/or washing conditions at 68° C. in 0.2×SSC, at 42° C. in 50% formamide, 4×SSC, or under conditions that afford levels of hybridization equivalent to those observed under either of these two conditions.

In still other embodiments, variants (including analogs, homologs and orthologs) of a candidate polypeptide can be identified by isolating variants of a candidate gene, e.g., using PCR with degenerate oligonucleotide primers designed on the basis of amino acid sequences of the candidate polypeptides and as described below.

Derivatives of a candidate polypeptide of the invention further include phosphorylated polypeptides, myristylated polypeptides, methylated polypeptides, and other candidate polypeptides that are chemically modified. Candidate polypeptides of the invention further include labeled variants; for example, radio-labeled with iodine or phosphorous (see, e.g., EP 372707B) or other detectable molecules such as, but by no means limited to, biotin, fluorescent dyes (e.g., Cy5 or Cy3), a chelating group complexed with a metal ion, a chromophore or fluorophore, a gold colloid, a particle such as a latex bead, or attached to a water soluble polymer such as poly(ethylene)-glycol (PEG). Chemical modifications of a candidate polypeptide may provide additional advantages under certain circumstances. See, for example, U.S. Pat. No. 4,179,337. For a review, see also Abuchowski et al., in Enzymes as Drugs (J. S. Holcerberg & J. Roberts, eds. 1981) pages 367-383. A review article describing protein modification and fusion proteins is also found in Fracis, Focus on Growth Factors 1992, 3:4-10, Mediscript: Mountview Court, Friern Barnet Lane, London N20, OLD, UK.

Candidate Nucleic Acids:

It is understood that, for purposes of describing the present invention, the term “candidate nucleic acid” refers to a nucleic acid comprising the nucleotide sequence of a candidate gene. For convenience, candidate nucleic acids of the present invention are frequently identified here by the SEQ ID number or GenBank Accession number for their preferred nucleotide sequences or for preferred amino acid sequences that they encode. However, it is understood that, as with the candidate polypeptides, the candidate nucleic acids of this invention are not limited to those particular sequences and include homologs and variants that are well within the ordinary skill of the art.

In general, candidate nucleic acid molecule of the present invention comprises a nucleic acid sequence that encodes a candidate polypeptide as defined, supra, the complement of a nucleic acid sequence that encodes a candidate polypeptide, and fragments thereof. Thus, the exemplary nucleic acid sequences provided in GenBank Accession numbers specified for particular candidate genes of the Examples, infra, represent preferred candidate nucleic acid sequences of the present invention.

In still other embodiments, the candidate nucleic acid molecules of the invention comprise nucleotide sequences that encode one or more domains of a candidate polypeptide.

The candidate nucleic acid molecules of the invention also include nucleic acids which comprise a sequence encoding one or more fragments of a candidate polypeptide sequence.

The candidate nucleic acid molecules of the invention also include nucleic acid molecules that comprise coding sequences for modified candidate polypeptides (e.g., having amino acid substitutions, deletions or truncations) and for variants (including allelic variants, analogs and homologs from the same or different species) candidate polypeptides. In preferred embodiments, such nucleic acid molecules have at least 50%, preferably at least 75% and more preferably at least 90% sequence identity to candidate polypeptide coding sequence (e.g., to the coding sequence set forth in the Examples, infra).

In addition, candidate nucleic acid molecules of the invention include ones that hybridize to another candidate nucleic acid molecule, e.g., in a Southern blot assay under defined conditions. For example, in specific embodiments a candidate nucleic acid molecule of the invention comprises a nucleotide sequence which hybridizes to a complement of a particular nucleic acid sequence, such as the coding sequence set forth in the GenBank Accession numbers for exemplary candidate genes specified in the Examples, infra. Alternatively, a nucleic acid molecule of the invention may hybridize, under the same defined hybridization conditions, to the complement of a fragment of a nucleotide sequence encoding a full length candidate polypeptide. Examples of preferred hybridization include those set forth above.

In other embodiments, the nucleic acid molecules of the invention comprise fragments of a full length candidate nucleic acid sequence. Such candidate nucleic acid fragments comprise a nucleotide sequence that corresponds to a sequence of at least 10 nucleotides, preferably at least 15 nucleotides and more preferably at least 20 nucleotides of a nucleotide sequence encoding a full length candidate polypeptide. In preferred embodiments, the candidate nucleic acid fragments comprise sequences of at least 10, preferably at least 15, and more preferably at least 20 nucleotides that are complementary and/or hybridize to a full length candidate nucleic acid sequence or to a fragment thereof. For hybridization with shorter nucleic acids, i.e., oligonucleotides, the position of mismatches becomes more important and the length of the oligonucleotide determines its specificity (see, Sambrook et al., supra, at 11.7-11.8). A minimum length for a hybridizable nucleic acid is preferably at least about 10 nucleotides, more preferably at least about 15 nucleotides, and still more preferably at least about 20 nucleotides.

Nucleic acid molecules comprising such fragments are useful, for example, as oligonucleotide probes and primers (e.g., PCR primers) to detect and amplify other nucleic acid molecules encoding a candidate polypeptide, including genes the encode variant candidate polypeptides. Oligonucleotide fragments of the invention may also be used, e.g., as antisense nucleic acids to modulate levels of a candidate gene's expression or transcription in cells.

The nucleic acid molecules of the invention also include “chimeric” nucleic acid molecules. Such chimeric nucleic acid molecules are polynucleotides which comprise at least one candidate nucleic acid sequence (which may be any of the full length or partial candidate nucleic acid sequences described above), and also at least one non-candidate nucleic acid sequence (i.e., a nucleic acid sequence not normally associated with the particular candidate gene). For example, the non-candidate nucleic acid sequence may be a heterologous regulatory sequence (for example a promoter sequence) that is derived from another gene and is not normally associated with the naturally occurring candidate gene. The non-candidate nucleic acid sequence may also be a coding sequence of another polypeptide such as FLAG, a histidine tag, glutathione S-transferase (GST), hemaglutinin, β-galactosidase, thioreductase or an immunoglobulin domain or domains (for examples, an Fc region). In preferred embodiments, a chimeric nucleic acid molecule of the invention encodes a fusion polypeptide of the invention.

Nucleic acid molecules of the invention, whether genomic DNA, cDNA or otherwise, can be isolated from any source including, for example, cDNA or genomic libraries derived from a cell or cell line from an organism that has the desired candidate gene. In the case of cDNA libraries, such libraries are preferably derived from a cell or cell line that expresses the particular candidate gene. Methods for obtaining candidate genes are well known in the art (see, e.g., Sambrook et al., 1989, supra).

The DNA may be obtained by standard procedures known in the art from cloned DNA (for example, from a DNA “library”), and preferably is obtained from a cDNA library prepared from tissues with high level expression of the protein. In one preferred embodiment, the DNA is obtained from a “subtraction” library to enrich the library for cDNAs of genes specifically expressed by a particular cell type or under certain conditions. Use of such a subtraction library may increase the likelihood of isolating cDNA for a particular gene. In still other embodiments, a library may be prepared by chemical synthesis, by cDNA cloning, or by the cloning of genomic DNA or fragments thereof purified from the desired cell (See, for example, Sambrook et al., 1989, supra; Glover, D. M. ed., 1985, DNA Cloning: A Practical Approach, MRL Press, Ltd. Oxford, U.K. Vols. I and II).

In one embodiment, a cDNA library may be screened for a desired candidate nucleic acid by identifying cDNA inserts that encode a polypeptide which is homologous or substantially similar to a candidate polypeptide of particular interest. Similarly, a cDNA library may be screened for a desired candidate nucleic acid by identifying cDNA inserts having a nucleic acid sequence that is homologous or substantially similar to a particular candidate nucleic acid sequence of interest.

Clones derived from genomic DNA may contain regulatory and intron DNA regions in addition to coding regions. Clones derived from cDNA generally will not contain intron sequences. Whatever the source, the gene is preferably molecularly cloned into a suitable vector for propagation of the gene. Identification of the specific DNA fragment containing the desired candidate gene may be accomplished in a number of ways. For example, a portion of a candidate gene can be purified and labeled to prepare a labeled probe (Benton & Davis, Science 1977, 196:180; Grunstein & Hogness, Proc. Natl. Acad. Sci. U.S.A. 1975, 72:3961). Those DNA fragments with substantial homology to the probe, such as an allelic variant from another individual, will hybridize. In a specific embodiment, highest stringency hybridization conditions are used to identify a homologous candidate gene.

The genes encoding derivatives and analogs of a candidate gene of this invention can be produced by various methods known in the art. The manipulations which result in their production can occur at the gene or protein level. For example, the cloned sequence can be modified by any of numerous strategies known in the art (Sambrook et al., 1989, supra). The sequence can be cleaved at appropriate sites with restriction endonuclease(s), followed by further enzymatic modification if desired, isolated, and ligated in vitro. In the production of the gene encoding a derivative or analog of a candidate gene, care should be taken to ensure that the modified gene remains within the same translational reading frame as the candidate gene from which it is derived, uninterrupted by translational stop signals, in the gene region where the desired activity is encoded.

Additionally, a candidate gene:sequence can be mutated in vitro or in vivo, to create and/or destroy translation, initiation, and/or termination sequences, or to create variations in coding regions and/or form new restriction endonuclease sites or destroy preexisting ones, to facilitate further in vitro modification. Modifications can also be made to introduce restriction sites and facilitate cloning the candidate gene into an expression vector. Any technique for mutagenesis known in the art can be used, including but not limited to, in vitro site-directed mutagenesis (Hutchinson, C., et al., J. Biol. Chem. 253:6551, 1978; Zoller and Smith, DNA 3:479-488, 1984; Oliphant et al., Gene 44:177, 1986; Hutchinson et al., Proc. Natl. Acad. Sci. U.S.A. 83:710, 1986), use of TAB″ linkers (Pharmacia Corp., Peapack, NJ), etc. PCR techniques are preferred for site directed mutagenesis (see Higuchi, 1989, “Using PCR to Engineer DNA”, in PCR Technology: Principles and Applications for DNA Amplification, H. Erlich, ed., Stockton Press, Chapter 6, pp. 61-70).

The identified and isolated gene can then be inserted into an appropriate cloning vector. A large number of vector-host systems known in the art may be used. Possible vectors include, but are not limited to, plasmids or modified viruses, but the vector system must be compatible with the host cell used. Examples of vectors include, but are not limited to, E. coli, bacteriophages such as lambda derivatives, or plasmids such as pBR322 derivatives or pUC plasmid derivatives, e.g., pGEX vectors, pmal-c, pFLAG, pKK plasmids (Clonetech, Palo Alto, Calif.), pET plasmids (Novagen, Inc., Madison, Wis.), pRSET or pREP plasmids, pcDNA (Invitrogen, Carlsbad, Calif.), or pMAL plasmids (New England Biolabs, Beverly, Mass.), etc. The insertion into a cloning vector can, for example, be accomplished by ligating the DNA fragment into a cloning vector which has complementary cohesive termini. However, if the complementary restriction sites used to fragment the DNA are not present in the cloning vector, the ends of the DNA molecules may be enzymatically modified. Alternatively, any site desired may be produced by ligating nucleotide sequences (linkers) onto the DNA termini. These ligated linkers may comprise specific chemically synthesized oligonucleotides encoding restriction endonuclease recognition sequences.

Recombinant molecules can be introduced into host cells via transformation, transfection, infection, electroporation, etc., so that many copies of the gene sequence are generated. Preferably, the cloned gene is contained on a shuttle vector plasmid, which provides for expansion in a cloning cell, e.g., E. coli, and facile purification for subsequent insertion into an appropriate expression cell line, if such is desired. For example, a shuttle vector, which is a vector that can replicate in more than one type of organism, can be prepared for replication in both E. coli and Saccharomyces cerevisiae by linking sequences from an E. coli plasmid with sequences from the yeast 2m plasmid.

It is understood that candidate nucleic acids of the invention may be either DNA or RNA and may be single-, double- or even triple-stranded (e.g., a triple-helix of candidate single-stranded candidate nucleic acids and/or their complement(s)). Candidate nucleic acids of the invention include genomic DNA, cDNA, RNA, mRNA, cRNA, etc.; as well as synthetic and genetically manipulated polynucleotides and both sense and antisense polynucleotides. Such synthetic polynucleotides include, for example, “protein nucleic acids” (PNA) formed by conjugating nucleotide bases to an amino acid backbone. Other exemplary synthetic nucleic acids include nucleic acids containing modified bases, such as thio-uracil, thio-guanine and fluoro-uracil. For convenience, the exemplary nucleotide sequences provided in this description are provided as sequences of DNA. However, it is understood that identical sequences of other types of nucleic acids (for example, RNA) may also be used and are equivalent. Thus, for example, where the particular nucleotide sequences in this description specify a thymine (T) at some position, it is understood that a uracil (U) may be substituted at that position and is a functional equivalent.

The polynucleotides of this invention may be flanked by natural regulatory sequences, or they may be associated with heterologous sequences such as promoters, enhancers, response elements, signal sequences, polyadenylation sequences, introns, 5′ and 3′-non-coding regions and the like. The term “heterologous”, in this context, refers to a combination of elements (e.g., sequences) that are not naturally occurring. Hence, a candidate nucleic acid of this invention may have sequences, such as a promoter etc., that are not normally associated with the candidate gene.

Nucleic acids of the invention may also be modified by any means known in the art. Non-limiting examples of such modifications include methylation, “caps”, substitution of one or more of the naturally occurring nucleotides with an analog, and internucleotide modifications such as, for example, those with uncharged linkages (e.g. methyl phosphonates, phosphotriesters, phosphoroamidates, carbamates, etc.) and with charged linkages (e.g., phosphorothioates, phosphorodithioates, etc.). Nucleic acids of the invention may contain one or more additional covalently linked moieties such as proteins (e.g., nucleases, toxins, antibodies, signal peptides, poly-L-lysine, etc.), intercalators (e.g., acridine, psoralen, etc.), chelators (e.g., metals, radioactive metals, iron, oxidative metals, etc.) and alkylators to name a few. The polynucleotides may be derivatized by formation of a methyl or ethyl phosphotriester or an alkyl phosphoramidite linkage. Furthermore, the polynucleotides herein may also be modified with a label capable of providing a detectable signal, either directly or indirectly. Exemplary labels include radioisotopes, fluorescent molecules, biotin and the like.

Expression of Candidate Polypeptides and Nucleic Acids:

A nucleotide sequence coding for candidate polypeptides, including chimeric proteins, antigenic fragments, derivatives or analogs thereof may be inserted into an appropriate expression vector, i.e., a vector which contains the necessary elements for the transcription and translation of the inserted protein-coding sequence. Thus, a nucleic acid encoding a candidate polypeptide of the invention can be operationally associated with a promoter in an expression vector of the invention. Both cDNA and genomic sequences can be cloned and expressed under control of such regulatory sequences. Such vectors can be used to express functional or functionally inactivated candidate polypeptides.

The necessary transcriptional and translational signals can be provided on a recombinant expression vector.

Potential host-vector systems include but are not limited to mammalian or other vertebrate cell systems transfected with expression plasmids or infected with virus (e.g., vaccinia virus, adenovirus, adeno-associated virus, herpes virus, etc.); insect cell systems infected with virus (e.g., baculovirus); microorganisms such as yeast containing yeast vectors; or bacteria transformed with bacteriophage, DNA, plasmid DNA, or cosmid DNA. The expression elements of vectors vary in their strengths and specificities. Depending on the host-vector system utilized, any one of a number of suitable transcription and translation elements may be used.

Expression of a candidate protein may be controlled by any promoter/enhancer element known in the art, but these regulatory elements must be functional in the host selected for expression. Promoters which may be used to control MIP-3α gene expression include, but are not limited to, cytomegalovirus (CMV) promoter (U.S. Pat. Nos. 5,385,839 and 5,168,062), the SV40 early promoter region (Benoist and Chambon, Nature 1981, 290:304-310), the promoter contained in the 3′ long terminal repeat of Rous sarcoma virus (Yamamoto, et al., Cell 1980, 22:787-797), the herpes thymidine kinase promoter (Wagner et al., Proc. Natl. Acad. Sci. U.S.A. 1981, 78:1441-1445), the regulatory sequences of the metallothionein gene (Brinster et al., Nature 1982, 296:39-42); prokaryotic expression vectors such as the b-lactamase promoter (Villa-Komaroff, et al., Proc. Natl. Acad. Sci. U.S.A. 1978, 75:3727-3731), or the tac promoter (DeBoer, et al., Proc. Natl. Acad. Sci. U.S.A. 1983, 80:21-25, 1983); see also “Useful proteins from recombinant bacteria” in Scientific American 1980, 242:74-94. Still other useful promoter elements which may be used include promoter elements from yeast or other fungi such as the Gal 4 promoter, the ADC (alcohol dehydrogenase) promoter, PGK (phosphoglycerol kinase) promoter, alkaline phosphatase promoter; and transcriptional control regions that exhibit hematopoietic tissue specificity, in particular: beta-globin gene control region which is active in myeloid cells (Mogram et al., Nature 1985, 315:338-340; Kollias et al., Cell 1986, 46:89-94), hematopoietic stem cell differentiation factor promoters, erythropoietin receptor promoter (Maouche et al., Blood 1991, 15:2557), etc.

In another embodiment, the invention provides methods for expressing candidate polypeptides by using a non-endogenous promoter to control expression of endogenous candidate genes within a cell. An endogenous candidate gene within a cell is a candidate gene of the present invention which is ordinarily (i.e., naturally) found in the genome of that cell. A non-endogenous promoter, however, is a promoter or other nucleotide sequence that may be used to control expression of a gene but is not ordinarily or naturally associated with the endogenous candidate gene. As an example, methods of homologous recombination may be employed (preferably using non-protein encoding nucleic acid sequences of the invention) to insert an amplifiable gene or other regulatory sequence in the proximity of an endogenous candidate gene. The inserted sequence may then be used, e.g. to provide for higher levels of the candidate gene's expression than normally occurs in that cell, or to overcome one or more mutations in the endogenous candidate gene's regulatory sequences which prevent normal levels of gene expression. Such methods of homologous recombination are well known in the art. See, for example, International Patent Publication No. WO 91/06666, published May 16, 1991 by Skoultchi; International Patent Publication No. WO 91/099555, published Jul. 11, 1991 by Chappel; and International Patent Publication No. WO 90/14092, published Nov. 29, 1990 by Kucherlapati and Campbell.

Soluble forms of the protein can be obtained by collecting culture fluid, or solubilizing inclusion bodies, e.g., by treatment with detergent, and if desired sonication or other mechanical processes, as described above. The solubilized or soluble protein can be isolated using various techniques, such as polyacrylamide gel electrophoresis (PAGE), isoelectric focusing, 2-dimensional gel electrophoresis, chromatography (e.g., ion exchange, affinity, immunoaffinity, and sizing column chromatography), centrifugation, differential solubility, immunoprecipitation, or by any other standard technique for the purification of proteins.

Preferred vectors are viral vectors, such as lentiviruses, retroviruses, herpes viruses, adenoviruses, adeno-associated viruses, vaccinia virus, baculovirus, and other recombinant viruses with desirable cellular tropism. Thus, a gene encoding a functional or mutant candidate protein or polypeptide domain fragment thereof can be introduced in vivo, ex vivo, or in vitro using a viral vector or through direct introduction of DNA. Expression in targeted tissues can be effected by targeting the transgenic vector to specific cells, such as with a viral vector or a receptor ligand, or by using a tissue-specific promoter, or both.

Antibodies to Candidate Gene Products:

Antibodies to candidate gene products of the present invention are useful, inter alia, for diagnostic and therapeutic methods, as set forth below. According to the invention, candidate polypeptides produced, e.g., recombinantly or by chemical synthesis, and fragments or other derivatives or analogs thereof, including fusion proteins, may be used as an immunogen to generate antibodies that recognize these polypeptides. Such antibodies include but are not limited to polyclonal, monoclonal, chimeric, single chain, Fab fragments, and an Fab expression library. Such an antibody is preferably specific for (i.e., specifically binds to) a human candidate polypeptide of the present invention. However, the antibody may, alternatively, be specific for an ortholog from some other species of organism, preferably another species of mammal such as mouse, rat or hamster, to name a few. The antibody may recognize wild-type, mutant or both forms of the candidate polypeptide.

Various procedures known in the art may be used for the production of polyclonal antibodies. For the production of polyclonal antibodies, various host animals can be immunized by injection with the desired candidate polypeptide, or derivatives (e.g., fragments or fusion proteins) thereof, including but not limited to rabbits, mice, rats, sheep, goats, etc. In one embodiment, the candidate polypeptide or fragment thereof can be conjugated to an immunogenic carrier, e.g., bovine serum albumin (BSA) or keyhole limpet hemocyanin (KLH). Various adjuvants may be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calinette-Guerin) and Corynebacterium parvum.

For preparation of monoclonal antibodies directed toward the candidate polypeptides, or fragment, analogs, or derivatives thereof, any technique that provides for the production of antibody molecules by continuous cell lines in culture may be used. These include but are not limited to the hybridoma technique originally developed by Kohler and Milstein (Nature 1975, 256:495-497), as well as the trioma technique, the human B-cell hybridoma technique (Kozbor et al., Immunology Today 1983, 4:72; Cote et al., Proc. Natl. Acad. Sci. U.S.A. 1983, 80:2026-2030), and the EBV-hybridoma technique to produce human monoclonal antibodies (Cole et al, in Monoclonal Antibodies and Cancer Therapy, Alan R. Liss, Inc., 1985, pp. 77-96). In an additional embodiment of the invention, monoclonal antibodies can be-produced in germ-free animals (International Patent Publication No. WO 89/12690). In fact, according to the invention; techniques developed for the production of “chimeric antibodies” (Morrison et al, J. Bacteriol. 1984, 159:870; Neuberger et al., Nature 1984, 312:604-608; Takeda et al., Nature 1985, 314:452-454) may also be used. Briefly, such techniques comprise splicing the genes from an antibody molecule from a first species of organism (e.g., a mouse) that is specific for a candidate polypeptide together with genes from an antibody molecule of appropriate biological activity derived from a second species of organism (e.g., from a human). Such chimeric antibodies are within the scope of this invention.

Antibody fragments which contain the idiotype of the antibody molecule can be generated by known techniques. For example, such fragments include but are not limited to: the F(ab′)₂ fragment which can be produced by pepsin digestion:of the antibody molecule; the Fab′ fragments which can be generated by reducing the disulfide bridges of the F(ab′)₂ fragment, and the Fab fragments which can be generated by treating the antibody molecule with papain and a reducing agent.

According to the invention, techniques described for the production of single chain antibodies (U.S. Pat. Nos. 5,476,786, 5,132,405, and 4,946,778) can be adapted to produce specific single chain antibodies that specifically bind to a particular candidate polypeptide. An additional embodiment of the invention utilizes the techniques described for the construction of Fab expression libraries (Huse et al., Science 1989, 246:1275-1281) to allow rapid and easy identification of monoclonal Fab fragments with the desired specificity for a candidate polypeptide, or for its derivatives, or analogs.

In the production and use of antibodies, screening for or testing with the desired antibody can be accomplished by techniques known in the art, e.g., radioimmunoassay, ELISA (enzyme-linked immunosorbant assay), “sandwich” immunoassays, immunoradiometric assays, gel diffusion precipitin reactions, immunodiffusion assays, in situ immunoassays (using colloidal gold, enzyme or radioisotope labels, for example), Western blots, precipitation reactions, agglutination assays (e.g., gel agglutination assays, hemagglutination assays), complement fixation assays, immunofluorescence assays, protein A assays; and immunoelectrophoresis assays, etc. In one embodiment, antibody binding is detected by detecting a label on the primary antibody. In another embodiment, the primary antibody is detected by detecting binding of a secondary antibody or reagent to the primary antibody. In a further embodiment, the secondary antibody is labeled. Many means are known in the art for detecting binding in an immunoassay and are within the scope of the present invention.

The foregoing antibodies can be used in methods known in the art relating to the localization and activity of a candidate polypeptide of interest, e.g., for Western blotting, imaging candidate polypeptides in situ, measuring levels thereof in appropriate physiological samples, etc. using any of the detection techniques mentioned above or known in the art. Such antibodies can also be used in assays for ligand binding, e.g., as described in U.S. Pat. No. 5,679,582. Antibody binding generally occurs most readily under physiological conditions, e.g., pH of between about 7 and 8, and physiological ionic strength. The presence of a carrier protein in the buffer solutions stabilizes the assays. While there is some tolerance of perturbation of optimal conditions, e.g., increasing or decreasing ionic strength, temperature, or pH, or adding detergents or chaotropic salts, such perturbations generally decrease binding stability.

In still other embodiments, antibodies may also be used to isolate cells which express a candidate polypeptide of interest (for example, OA chondrocyte cells) by panning or related immunoadsorption techniques.

In a specific embodiment, antibodies that agonize or antagonize the activity of a candidate polypeptide can be generated. In particular; intracellular single chain Fv antibodies can be used to regulate (inhibit) MIP-3a activity (Marasco et al., Proc. Natl. Acad. Sci. U.S.A. 1993, 90:7884-7893; Chen., Mol. Med. Today 1997, 3:160-167; Spitz et al., Anticancer Res. 1996, 16:3415-22; Indolfi et al. Nat. Med. 1996, 2:634-635; Kijma et al., Pharmacol. Ther. 1995, 68:247-267). Such antibodies can be tested using the assays described infra for identifying ligands.

Applications and Uses:

Described herein are various applications and uses for candidate genes and gene products that are identified in screening methods of the present invention. These include, inter alia, applications and uses for the candidate nucleic Acids and polypeptides described above, including the particular candidate nucleic acids and polypeptides provided in the examples as well as fragments, analogs, homologs and other variants thereof.

The candidate genes and gene products that are identified in screening assays of this invention include ones that are expressed at elevated levels in cells from patients with OA compared to healthy subjects. Other candidate genes and gene products of the invention induce one or more features of an OA phenotype when they are expressed in cells. Hence, candidate genes and/or gene products may be used as tissue-specific markers to detect and/or identify OA cells or tissue, including OA chondrocyte cells and cartilage. Candidate nucleic acids and polypeptides of the invention can therefore be used in methods for detecting OA, e.g., in diagnostic and prognostic applications, by using one or more candidate genes or gene products to detect expression in a sample such as a cell or tissue sample from an individual (obtained, e.g., from a biopsy).

In addition, candidate genes and gene products of the invention can serve as drug targets for the development of therapeutics to treat individuals suffering from OA. Methods are provided that use candidate nucleic acids and polypeptides of the invention to screen for compounds that can be used to treat or prevent cartilage degradation, as well as for the treatment or prevention of conditions such as OA. Such screening methods may, for example, identify compounds that modulate or interfere with binding of a candidate gene or gene product to its ligand or receptor. In other embodiments, drug screening methods of the invention may identify compounds that modulate downstream signaling events from a candidate or gene or gene product, or they may identify compounds that interfere with upstream signaling event that activate a candidate gene or gene product. In still other embodiments, drug screening assays of the invention may identify compounds that inhibit the expression and/or activity of either a candidate gene or its gene product.

Drug screening assays. Using screening assays such as those described below, it is possible to identify compounds that bind to or otherwise interact with candidate genes of the present invention and/or their gene products, including intracellular compounds (for example, proteins or portions of proteins), natural and synthetic ligands or receptors, compounds that interfere with the interaction of a candidate gene product (for example, compounds that interfere with specific binding of a candidate gene product to its receptor or ligand), and compounds that modulate the activity of a candidate gene (for example, by modulating the level of the candidate gene's expression) or the activity (for example, the bioactivity) of a candidate gene product.

The screening assays of this invention may therefore be used to identify compounds that specifically bind to a candidate gene or gene product to modulate its expression. For example, the screening assays described here may be used to identify compounds that bind to a promoter or other regulatory sequence of a candidate gene, and so may modulate the level of that candidate gene's expression (see, for example, Platt, J. Biol Chem. 1994, 269:28558-28562). The screening assays may also be used to identify compounds that bind to and thereby stabilize a candidate nucleic acid or polypeptide. In addition, these screening assays may be used to identify compounds that inhibit or modulate such binding interactions and which are therefore useful, e.g., as agonists or antagonists for the candidate gene product's binding to a specific transcription factor or enhancer, or for the candidate gene product's binding to a stabilizer. Compounds identified in these or similar screening assays may therefore be used to treat diseases and disorders that are associated with the candidate gene's abnormal expression and/or activity, associated with, but not limited to, OA.

Classes of compounds that may be identified by such screening assays include, but are not limited to, small molecules (e.g., organic or inorganic molecules which are less than about 2 kDa in molecular weight, are more preferably less than about 1 kDa in molecular weight, and/or are able to cross the blood-brain barrier or gain entry into an appropriate cell and affect expression of either a candidate gene or of some gene involved in the candidate gene's regulatory pathway) as well as macromolecules (e.g., molecules greater than about 2 kDa in molecular weight). Compounds identified by these screening assays may also include nucleic acids, peptides and polypeptides. Examples of such compounds (including peptides) include but are not limited to: soluble peptides; fusion peptide members of combinatorial libraries (such as ones described by Lam et al., Nature 1991, 354:82-84; and by Houghten et al., Nature 1991, 354:84-86); members of libraries derived by combinatorial chemistry, such as molecular libraries of D- and/or L-configuration amino acids; phosphopeptides, such as members of random or partially degenerate, directed phosphopeptide libraries (see, e.g., Songyang et al., Cell 1993, 72:767-778); antibodies, including but not limited to polyclonal, monoclonal, humanized, anti-idiotypic, chimeric or single chain antibodies; antibody fragments, including but not limited to Fab, F(ab′)₂, Fab expression library fragments, and epitope-binding fragments thereof. Nucleic acids used in these screening assays may be DNA or RNA, or synthetic nucleic acids. Particular examples include, but are by no means limited to, antisense nucleic acids and ribozymes, as well as double-stranded and triple helix nucleic acid molecules.

Assays for binding compounds. In vitro systems can be readily designed to identify compounds capable of binding to a candidate gene product of the present invention. Such compounds can be useful, for example, in modulating the expression, stability or activity of a wild-type candidate gene product or, alternatively, to modulate the expression, stability or activity of a mutant or other variant candidate gene product.

Generally, such screening assays involve preparation of a reactive mixture comprising the candidate gene product of interest and a test compound under conditions and for a time sufficient to allow the two compounds to interact (e.g., bind), thereby forming a complex that may be detected. The assays may be conducted in any of a variety of different ways. For example, one embodiment comprises anchoring a candidate polypeptide or a test compound onto a solid phase and detecting complexes of the candidate polypeptide and the test compound that are on the solid phase at the end of the reaction and after removing (e.g., by washing) unbound compounds. For example, in one preferred embodiment of such a method, a candidate gene product may be anchored onto a solid surface and a labeled compound (e.g., labeled according to any of the methods described supra) is contacted to the surface. After incubating the test compound for a sufficient time and under sufficient conditions that a complex may form between the candidate gene product and the test compound, unbound molecules of the test compound are removed from the surface (e.g., by washing) and labeled molecules which remain are detected.

In another, alternative embodiment, molecules of one or more different test compounds are attached to the solid phase and molecules of a labeled candidate polypeptide may be contacted thereto. In such embodiments, the molecules of different test compounds are preferably attached to the solid phase at a particular location on the solid phase so that test compounds that bind to the candidate polypeptide may be identified by determining the location of the bound candidate polypeptides on the solid phase or surface.

Assays for compounds that interact with a candidate gene or gene product. Any of a variety of known methods for detecting protein-protein interactions may also be used to detect and/or identify proteins that interact with a candidate gene product of the invention. For example, co-immunoprecipitation, cross-linking and co-purification through gradients or chromatographic columns as well as other techniques known in the art may be employed. Proteins which may be identified using such assays include, but are not limited to, extracellular proteins, such as receptors and ligands for candidate genes and/or their gene products, as well as intracellular proteins such as signal transducing proteins.

Compounds, including other cellular proteins and nucleic acids, that interact with a candidate gene or gene product may themselves be used in the methods of this invention, e.g., to modulate activity of the candidate gene or gene product and to treat or prevent cartilage degradation. Alternatively, such interacting compounds may, themselves, be used in the screening assays of this invention to identify other compounds that could, in turn, be used to treat or prevent cartilage degradation.

As an example, and not by way of limitation, an expression cloning assay may be used to identify receptors and other proteins that specifically interact with a candidate gene product of interest. In such assays, a cDNA expression library may be generated from any cell line that expresses such a receptor. Clones from such an expression library may then be transfected or infected into cells that do not normally express a receptor for the candidate gene product. Cells that are transfected with a clone that encodes a receptor which specifically binds to the candidate gene product may then express this receptor, and can be identified and isolated using standard techniques such as FACS or using magnetic beads that have the candidate polypeptide (for example, an Fc-fusion of the candidate polypeptide) attached thereto.

Alternatively, receptors and/or ligands that specifically bind to a candidate gene product may be isolated from a cell line using immunoprecipitation techniques that are well known in the art.

Receptors and/or ligands for a candidate gene product may also be isolated using any of the screening assays discussed, supra for identifying binding compounds. For example, an Fc-fusion polypeptide of a candidate gene product may be bound or otherwise attached to a solid surface, and a labeled compound (e.g., a candidate receptor or ligand) may be contacted to the surface for a sufficient time and under conditions that permit formation of a complex between the fusion polypeptide and the test compound. Unbound molecules of the test compound can then be removed from the surface (e.g., by washing), and labeled compounds that remain bound can be detected.

Once so isolated, standard techniques may be used to identify any protein detected in such assays. For example, at least a portion of the amino acid sequence of a protein that interacts with a candidate gene product can be ascertained using techniques well known in the art, such as the Edman degradation technique (see, e.g., Creighton, 1983, Proteins: Structures and Molecular Principles, W.H. Freeman&Co., New York, pages 34-49).

Once such proteins have been identified, their amino acid sequence may be used as a guide for the generation of oligonucleotide mixtures to screen for gene sequences encoding such proteins; e.g., using standard hybridization or PCR techniques described supra. See, for example, Ausubel supra; and PCR Protocols: A Guide to Methods and Applications, Innis et al., eds., Academic Press, Inc., New York (1990) for descriptions of techniques for the generation of such oligonucleotide mixtures and their use in screening assays.

Other methods are known in the art which result in the simultaneous identification of genes that encode a protein that interacts with a candidate gene or gene product. For example, expression libraries may be probed with a labeled candidate polypeptide.

As another example and not by way of limitation, a two-hybrid system may be used to detect protein interactions with a candidate gene product in vivo. Briefly, utilizing such a system, plasmids may be constructed which encode two hybrid proteins, one of which preferably comprises of the DNA-binding domain of a transcription activator protein fused to a candidate gene product. The other hybrid protein preferably comprises an activation domain of the transcription activator protein used in the first hybrid, fused to an unknown protein that is encoded by a cDNA recombined into the plasmid library as part of a cDNA library. Both the DNA-binding domain fusion plasmid and the cDNA library may be co-transformed into a strain of Saccharomyces cerevisiae or other suitable organism which contains a reporter gene (for example, HBS, lacZ, HIS3 or GFP). Preferably, the regulatory region of this reporter gene comprises a binding site for the transcription activator moiety of the two hybrid proteins. In such a two-hybrid system, the presence of either of the two hybrid proteins alone cannot activate transcription of the reporter gene. Specifically, the DNA-binding domain hybrid protein cannot activate transcription because it cannot localize to the necessary activation function. Likewise, the activation domain hybrid protein cannot activate transcription because it cannot localize to the DNA binding site on the reporter gene. However, interaction between the two hybrid proteins, reconstitutes that functional transcription activator protein and results in expression of the reporter gene. Thus, in a two-hybrid system such as the one described here in detail, an interaction between a candidate polypeptide (i.e., the candidate polypeptide fused to the transcription activator's DNA binding domain) and a test polypeptide (i.e., a protein fused to the transcription activator's DNA binding domain) may be detected by simply detecting expression of a gene product of the reporter gene.

cDNA libraries for screening in such two-hybrid and other assays may be made according to any suitable technique known in the art. As a particular and non-limiting example, cDNA fragments may be inserted into a vector so that they are translationally fused to the transcriptional activation domain of GAL4, and co-transformed along with a “bait” GAL4 fusion plasmid (encoding a GAL4-fusion of a candidate gene product) into a strain of Saccharomyces cerevisiae or other suitable organism that contains a HIS3 gene driven by a promoter that contains a GAL4 activation sequence. A protein from this cDNA library, fused to the GAL4 transcriptional activation domain, which interacts with the candidate polypeptide moiety of the GAL4-fusion will reconstitute and active GAL4 protein, and can thereby drive expression of the HIS3 gene. Colonies that express the HIS3 gene may be detected by their growth on petri dishes containing semi-solid agar based media lacking histidine. The cDNA may then be purified from these strains, sequenced and used to identify the encoded protein which interacts with the candidate polypeptide.

Once compounds have been identified which bind to a candidate gene or gene product of the invention, the screening methods described in these methods may also be used to identify other compounds (e.g., small molecules, peptides and proteins) which bind to these binding compounds. Such compounds may also be useful for modulating bioactivities associated with a candidate gene and its gene product, for example by binding to a natural receptor, ligand or other binding partner and preventing its interaction with the candidate gene product. For instance, these compounds could be tested for their ability to inhibit the binding of an Fc-fusion of the candidate gene product to cell lines which express a specific receptor for the candidate gene product.

Assays for compounds that interfere with a candidate gene/protein ligand interaction. As noted supra, a candidate gene product of the invention may interact with one or more molecules (e.g., with a specific receptor or ligand) in vivo or in vitro. Compounds that disrupt or otherwise interfere with this binding interaction are therefore useful in modulating biological activity or activities that are associated with the candidate gene product, including for example, cartilage degradation. Such compounds may therefore be useful, e.g., to treat disorders such as OA that are associated with abnormal levels of a candidate gene or gene product's expression and/or activity.

Such compounds include, but are not limit to, compounds identified according to the screening assays described supra, for identifying compounds that bind to a candidate gene product, including any of the numerous exemplary classes of compounds described therein.

In general, assays for identifying compounds that interfere with the interaction between a candidate gene product and a binding partner (e.g., a receptor or ligand) involve preparing a test reaction mixture that contains the candidate gene product and its binding partner under conditions and for a time sufficient for the candidate gene product and its binding partner to bind and form a complex. In order to test a compound for inhibitory activity (i.e., for the ability to inhibit formation of the binding complex or to disrupt the binding complex once formed), the test compound preferably is also present in the test reaction mixture. In one exemplary embodiment, the test compound may be initially included in the test reaction mixture with the candidate gene product and its binding partner. Alternatively, however, the test compound may be added to the test reaction mixture at a later time, subsequent to the addition of the candidate gene product and its binding partner. In preferred embodiments, one or more control reaction mixtures, which do not contain the test compound, may also be prepared. Typically, a control reaction mixture will contain the same candidate gene product and binding partner that are in the test reaction mixture, but will not contain a test compound. A control reaction mixture may also contain a placebo, not present in the test reaction mixture, in place of the test compound. The formation of a complex between the candidate gene product and the binding partner may then be detected in the reaction mixture. The formation of such a complex in the absence of the test compound (e.g., in a control reaction mixture) but not in the presence of the test compound, indicates that the test compound is one which interferes with or modulates the interaction of the candidate polypeptide and its binding partner.

Such assays for compounds that modulate the interaction of a candidate gene product and a binding partner may be conducted in a heterogeneous format or, alternatively, in a homogeneous format. Heterogeneous assays typically involve anchoring either a candidate gene product or a binding partner onto a solid phase and detecting compounds anchored to the solid phase at the end of the reaction. Thus, such assays are similar to the solid phase assays described supra for detecting and/or identifying candidate nucleic acids and gene products and for detecting or identifying binding partners. Indeed, those skilled in the art will recognize that many of the principles and techniques described above for those assays may be modified and applied without undue experimentation in the solid phase assays described here, for identifying compounds that modulate interaction(s) between a candidate gene product and a binding partner.

Regardless of the particular assay used, the order to which reactants are added to a reaction mixture may be varied; for example, to identify compounds that interfere with the interaction of a candidate gene product with a binding partner by competition, or to identify compounds that disrupt a preformed binding complex. Compounds that interfere with the interaction of a candidate gene product with a binding partner by competition may be identified by conducting the reaction in the presence of a test compound. Specifically, in such assays a test compound may be added to the reaction mixture prior to or simultaneously with the candidate gene product and the binding partner. Test compounds that disrupt preformed complexes of a candidate gene product and a binding partner may be tested by adding the test compound to a reaction mixture after complexes have been formed.

The screening assays described herein may also be practiced using peptides or polypeptides that correspond to portions of a full length candidate polypeptide or protein, or with fusion proteins comprising such peptide or polypeptide sequences. For example, screening assays for identifying compounds the modulate interactions of a candidate polypeptide with a binding partner may be practiced using peptides or polypeptides corresponding to particular regions or domains of a full length candidate polypeptide that bind to a binding partner (e.g., receptor “binding sites”).

A variety of methods are known in the art that may be used to identify specific binding sites of a candidate polypeptide. For example, binding sites may be identified by mutating a candidate gene and screening for disruptions of binding as described above. A gene encoding the binding partner may also be mutated in such assays to identify mutations that compensate for disruptions from the mutation to the candidate gene. Sequence analysis of these mutations can then reveal mutations that correspond to the binding region of the two proteins.

In an alternative embodiment, a protein (e.g. a candidate protein or a protein binding partner to a candidate protein) may be anchored to a solid surface or support using the methods described hereinabove. Another labeled protein which binds to the protein anchored to the solid surface may be treated with a proteolytic enzyme, and its fragments may be allowed to interact with the protein attached to the solid surface, according to the methods of the binding assays described supra. After washing, short, labeled peptide fragments of the treated protein may remain associated with the anchored protein. These peptides can be isolated and the region of the fall length protein from which they are derived may be identified by the amino acid sequence.

In still other embodiments, compounds that interfere with interactions between a candidate polypeptide and a receptor or ligand may also be identified by screening for compounds that modulate binding of the candidate polypeptide (for example, an Fc-fusion construct of the candidate polypeptide) to cells that express a specific receptor thereto.

Diagnostic and Prognostic Applications:

A variety of methods can be employed for diagnostic and prognostic methods using reagents such as the candidate nucleic acids and polypeptides described supra as well as antibodies directed against such candidate nucleic acids and polypeptides. For example, using the methods described here it is possible to detect expression of a candidate nucleic acid or protein in a biological sample from an individual, such as in cells or tissues in a sample (e.g., from a biopsy) obtained or derived from an individual subject or patient. As explained above, candidate nucleic acids and polypeptides identified in screening assays of this invention induce one or more characteristics associated with OA when they are expressed in cells. Hence, the expression of such candidate nucleic acids and/or polypeptides at elevated levels in cells is an indication of OA or a related disorder.

Using the methods described here (as well as other methods known in the art) a skilled artisan may detect elevated levels of a candidate nucleic acid or polypeptide in a sample of cells or tissue from an individual, and may thereby detect and/or identify cells or tissue in that sample as being symptomatic of OA. In certain preferred embodiments the particular type of tissue identified in such methods is cartilage tissue. By using such methods to detect such cells or tissue in an individual, a skilled user may thereby diagnose the presence of OA in that individual.

In preferred embodiments the methods described herein are performed using pre-packaged diagnostic kits. Such kits may comprise at least one specific candidate nucleic acid or a candidate gene product specific antibody reagent. For example, said diagnostic kit may be used for detecting mRNA levels or protein levels of a candidate gene or gene product selected from the group consisting of those disclosed in Table V and Table VI, said kit comprising: (a) a polynucleotide of said candidate gene or a fragment thereof; (b) a nucleotide sequence complementary to that of (a); (c) an expression product of said candidate gene, or a fragment thereof; or (d) an antibody to said expression product and wherein components (a), (b), (c) or (d) may comprise a substantial component.

In preferred embodiments, a kit will also contain instructions for its use, e.g., to detect diseased cells or tissues, or to diagnose a disorder (such as OA) associated with abnormal expression of a candidate gene or gene product. In preferred embodiments, such instructions may be packaged directly with the kit. In other embodiments, however, instructions may be provided separately. For example, the invention provides embodiments of kits where instructions for using the kit may be downloaded, e.g., from the internet. A kit of the invention may also comprise, preferably in separate containers, suitable buffers and other solutions to use the reagents (e.g., nucleic acid or antibody specific for a candidate gene or gene product) to detect the candidate gene or gene product. The kit and any reagent(s) contained therein may be used, for example, in a clinical setting, to diagnose patients exhibiting or suspected of having OA.

A sample comprising a cell of any cell type or tissue of any tissue type in which a candidate gene is expressed may also be used in such diagnostic methods, e.g., for detection of candidate gene expression or of candidate gene products (such as candidate polypeptides), as well as for identifying cells, e.g. chondrocytes, that express a candidate gene or a candidate gene product. Thus, in one embodiment, the methods described herein may be performed in situ, e.g., using cells or tissues obtained from an individual such as in a biopsy. Such methods may be useful, for example, in surgical procedures where it is desirable to identify arthritic tissue without removing benign, healthy tissue.

The methods described herein are not limited to diagnostic applications, but may also be used in prognostic applications, e.g., to monitor the progression of a disease (such as OA) that is associated with abnormal expression of a candidate gene or gene product, or to monitor a therapy thereto. Accordingly, prognostic methods of the invention may comprise, in one exemplary embodiment, monitoring candidate nucleic acid or polypeptide levels in an individual during the course of a treatment or therapy (for example, a drug treatment or exercise regimen) for OA. Similarly, the methods of the invention may also be used to detect and identify diseased cells and tissue (e.g. cells overexpressing one or more candidate genes of gene products compared to non OA cells or tissue) during the course of a therapy. In such embodiments, decreasing numbers of diseased cells is generally indicative of an effective treatment. The methods of the invention may further be used, e.g., to screen candidate drugs or compounds and identify ones that may be effective, e.g., as anti-OA drugs. Such methods may be performed in vivo (e.g., using an animal model) or in vitro (for example, in a cell culture assay). In one embodiment such methods may comprise contacting a candidate compound to a cell and identifying whether expression of a candidate gene or gene product by the cell has been inhibited. In another embodiment, a compound may be contacted to a cell or administered to an organism and extracellular levels of candidate nucleic acid or polypeptide may be measured (for example, in cell culture media for cell culture assays, or in blood or other body fluid in an animal model assay).

Detection of candidate nucleic acids. The diagnostic and prognostic methods of the invention include methods for assaying the level of candidate gene expression. A variety of methods known in the art may be used to detect assay levels of one or more candidate nucleic acid sequences in a sample. For example, RNA from a cell type or tissue that is known or suspected to express one or more candidate genes of interest may be isolated and tested utilizing hybridization or PCR techniques known in the art. The isolated cells may be, for example, cells derived from a cell culture or from an individual. The analysis of cells taken from a cell culture may be useful, e.g., to test the effect of compounds on the expression of one or more candidate genes, or alternatively, to verify that the cells are ones of a particular cell type that express one or more candidate genes of interest.

As an example, and not by way of limitation, diagnostic methods for the detection of candidate nucleic acids can involve contacting and incubating nucleic acids (including recombinant DNA molecules, cloned genes or degenerate variants thereof) obtained from a sample with one or more labeled nucleic acid reagents, such as recombinant candidate DNA molecules, cloned genes or degenerate variants thereof, under conditions favorable for specifically annealing or hybridizing these reagents to their complementary sequences in the sample nucleic acids. After incubation, all non-annealed or non-hybridized nucleic acids are removed. The presence of nucleic acids that have hybridized, if any such molecules exist, is then detected and the level of candidate nucleic acid sequences to which the nucleic acid reagents have annealed may be compared to the annealing pattern or level expected from a control sample (e.g., from a sample of normal, non-OA cells or tissues) to determine whether candidate nucleic acid is expressed at an elevated level.

In a preferred embodiment of such a detection scheme, the nucleic acid from the cell type or tissue of interest may be immobilized, for example, to a solid support such as a membrane or a plastic surface (for example, on a nylon membrane, a microtiter plate or on polystyrene beads). After incubation, non-annealed, labeled candidate nucleic acid reagents maybe easily removed and detection of the remaining, annealed, labeled candidate nucleic acid reagents may be accomplished using standard techniques that are well-known in the art.

Alternative diagnostic methods for the detection of candidate nucleic acids in patient samples or in other cell or tissue sources may involve their amplification, e.g., by PCR (see, for example, the experimental embodiment taught in U.S. Pat. No. 4,683,202) followed by detection of the amplified molecules using techniques that are well known to those of skilled in the art. The resulting level of amplified candidate nucleic acids may be compared to those levels that would be expected if the sample being amplified contained only normal levels of the candidate nucleic acid(s), as normal cells or tissues, to determine whether elevated levels of any candidate nucleic acid(s) are expressed.

In one preferred embodiment of such a detection scheme, a cDNA molecule is synthesized from an RNA molecule of interest (e.g., by reverse transcription). A sequence within the cDNA may then be used as a template for a nucleic acid amplification reaction such as PCR. Nucleic acid reagents used as synthesis initiation reagents (e.g., primers) in the reverse transcription and amplification steps of such an assay are preferably chosen from the candidate nucleic acid sequences described herein or are fragments thereof. Preferably, the nucleic acid reagents are at least about 9 to 30 nucleotides in length. The amplification may be performed using, e.g., radioactively labeled or fluorescently labeled nucleotides, for detection. Alternatively, enough amplified product may be made such that the product can be visualized by standard ethidium bromide or other staining methods.

Candidate gene expression assays of the invention may also be performed in situ (i.e., directly upon tissue sections of patient tissue, which may be fixed and/or frozen), thereby eliminating the need for nucleic acid purification. Candidate nucleic acid reagents may be used as probes or as primers for such in situ procedures (see, for example, Nuovo, PCR In Situ Hybridization: Protocols And Application, 1992, Raven Press, New York). Alternatively, if a sufficient quantity of the appropriate cells can be obtained, standard Northern analysis can be performed to determine the level of candidate gene expression by detecting levels of one or more candidate mRNAs.

Detection of candidate gene products. The diagnostic and prognostic methods of the invention also include ones that comprise detecting levels of a candidate polypeptide and including functionally conserved variants and fragments thereof. For example, antibodies directed against unimpaired, wild-type or mutant candidate gene products or against functionally conserved variants or peptide fragments of a candidate gene product may be used as diagnostic and prognostic reagents. Such reagents may be used, for example, to detect abnormalities in the level of candidate gene product synthesis or expression, or to detect abnormalities in the structure, temporal expression or physical location of a candidate gene product. Antibodies and immunoassay methods such as those described hereinbelow also have important in vitro applications for assessing the efficacy of treatments, e.g., for OA. For example, antibodies, or fragments of antibodies, can be used in screens of potentially therapeutic compounds in vitro to ascertain a compound's effects on candidate gene expression and candidate polypeptide production. Compounds that may have beneficial effects on a disorder associated with abnormal candidate gene expression can be identified and a therapeutically effective dose for such compounds may be determined using such assays.

As one example, antibodies or fragments of antibodies may be used to detect the presence of a candidate gene product, a variant of a candidate gene product or fragments thereof, for example, by immunofluorescence techniques employing a fluorescently labeled antibody coupled with light microscopic, flow cytometric or fluorimetric detection methods.

In particularly preferred embodiments, antibodies or fragments thereof may also-be employed histologically, for example in immunofluorescence or immunoelectron microscopy techniques, for in situ detection of a candidate gene product. In situ detection may be accomplished by removing a histological specimen (e.g., a tissue sample) from a patient and applying thereto a labeled antibody of the present invention or a fragment of such an antibody. The antibody or antibody fragment is preferably applied by overlaying the labeled antibody or antibody fragment onto a biological sample. Through the use of such a procedure, it is possible to detect, not only the presence of a candidate gene product, but also the gene product's distribution in the examined tissue. A wide variety of histological methods that are well known in the art (for example, staining procedures) can be readily modified by those skilled in the art without undue experimentation to achieve such in situ detection. Immunoassays for candidate gene products will typically comprise incubating a biological sample (for example, a tissue extract) in the presence of a detectably labeled antibody that is capable of specifically binding a candidate gene product (including, for example, a functionally conserved variant or a peptide fragment thereof). The bound antibody may then be detected by any of a number of techniques well known in the art.

Therapeutic Methods and Pharmaceutical Compositions:

Candidate nucleic acids and polypeptides, and specific antibodies thereto may also be used in therapeutic methods and compositions, e.g., to treat, prevent or ameliorate diseases and disorders associated with abnormal (preferably elevated) levels of the candidate gene's expression. In preferred embodiments such methods are used to treat OA. In one preferred embodiment the therapeutic methods of the invention comprise administering one or more compounds that modulate (e.g., inhibit) the expression or activity of a candidate gene or its gene product; for example, compounds that bind to a candidate nucleic acid or polypeptide of the invention, compounds that modulate expression of a candidate gene, and/or compounds that interfere with or modulate binding of a candidate nucleic acid or polypeptide with a binding compound.

In another preferred embodiment, the therapeutic methods of the invention may comprise one or more cell-targeted therapies which target compounds (for example, drugs, pro-drugs, toxins or cytotoxins) to cells expressing a candidate nucleic acid or polypeptide.

Inhibitory approaches. In alternative embodiments, the present invention provides methods and compositions for treating a disease or disorder (for example, OA) associated with the abnormal expression or activity of a candidate gene or gene product by modulating (e.g., increasing or decreasing) the expression or activity of the candidate gene or its gene product. Such methods may simply comprise administering one or more compounds that modulate expression of a candidate gene, synthesis of a candidate gene product or activity of a candidate gene product so the immune response is modulated (e.g., enhanced or suppressed). Preferably, these one or more compounds are administered until one or more symptoms of the disorder are eliminated or at least ameliorated.

Among the compounds that may exhibit an ability to modulate the activity, expression or synthesis of a candidate nucleic acid are antisense molecules. Such molecules may be designed to reduce or inhibit wild-type nucleic acids and polypeptides or, alternatively, may target mutant candidate nucleic acids or polypeptides.

Antisense RNA and DNA molecules act to directly block the translation of mRNA by hybridizing to target mRNA molecules and preventing protein translation. Antisense approaches involve the design of oligonucleotides that are complementary to a target gene mRNA. The antisense oligonucleotides will bind to the complementary target gene mRNA transcripts and prevent translation. Absolute complementarity, although preferred, is not required. As used in this description, “antisense” broadly includes RNA-RNA interactions, triple helix interactions, ribozymes and RNase-H mediated arrest. Antisense nucleic acid molecules can be encoded by a recombinant gene for expression in a cell (see, e.g., U.S. Pat. Nos. 5,814,500; and 5,811,234) or, alternatively, they can be prepared synthetically (U.S. Pat. No. 5,780,607).

A sequence that is “complementary” to a portion of a nucleic acid refers to a sequence having sufficient complementarity to be able to hybridize with the nucleic acid and form a stable duplex. The ability of nucleic acids to hybridize will depend both on the degree of sequence complementarity and the length of the antisense nucleic acid. Generally, however, the longer the hybridizing nucleic acid, the more base mismatches it may contain and still form a stable duplex (or triplex in triple helix methods). A tolerable degree of mismatch can be readily ascertained, e.g., by using standard procedures to determine the melting temperature of a hybridized complex.

In one preferred embodiment, oligonucleotides complementary to non-coding regions of a candidate gene may be used in an antisense approach to inhibit translation of endogenous candidate mRNA molecules. Antisense nucleic acids are preferably at least six nucleotides in length, and more preferably range from between about six to about 50 nucleotides in length. In specific embodiments, the oligonucleotides may be at least 10, at least 15, at least 20, at least 25 or at least 50 nucleotides in length.

It is generally preferred that in vitro studies are first performed to quantitate the ability of an antisense oligonucleotide to inhibit gene expression. It is preferred that these studies utilize controls that distinguish between antisense gene inhibition and nonspecific biological effects of oligonucleotides. It is also preferred that these studies compare levels of the target RNA or protein with that of an internal control RNA or protein. Additionally, it is envisioned that results obtained using the antisense oligonucleotide are compared with those obtained using a control oligonucleotide. It is preferred that the control oligonucleotide is of approximately the same length as the test oligonucleotide and that the nucleotide sequence of the oligonucleotide differs from the antisense sequence no more than is necessary to prevent specific hybridization to the target sequence.

While antisense nucleotides complementary to the target gene coding region sequence could be used, those complementary to the transcribed, untranslated region are most preferred.

Antisense molecules are preferably delivered to cells, such as chondrocytes, that express the target gene in vivo. A number of methods have been developed for delivering antisense DNA or RNA to cells. For example, antisense molecules can be injected directly into the tissue site (e.g., directly into a tumor), or modified antisense molecules can be designed to target the desired cells (e.g., antisense linked to peptides or antibodies that specifically bind receptors or antigens expressed on the target cell surface) can be administered systemically.

Preferred embodiments achieve intracellular concentrations of antisense nucleic acid molecules which are sufficient to suppress translation of endogenous mRNAs. For example, one preferred approach uses a recombinant DNA construct in which the antisense oligonucleotide is placed under the control of a strong pol III or pol II promoter. The use of such a construct to transfect target cells in the patient will result in the transcription of sufficient amounts of single stranded RNAs that will form complementary base pairs with the endogenous target gene transcripts and thereby prevent translation of the target gene mRNA. For example, a vector, as set forth above, can be introduced e.g., such that it is taken up by a cell and directs the transcription of an antisense RNA. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in mammalian cells. Expression of the sequence encoding the antisense RNA can be by any promoter known in the art to act in the particular cell type (for example in a hemopoietic cell). For example, any of the promoters discussed supra in connection with the expression of recombinant candidate nucleic acids can also be used to express a candidate antisense nucleic acid.

In addition to antisense technology, RNA aptamers (Good et al., 1997, Gene Therapy 4: 45-54), double stranded RNA (WO 99/32619), ribozymes (Cech. J., 1988, Amer. Med Assn. 260:3030; Cotten et al., 1989, EMBO J. 8:3861-3866; Grassi and Marini, 1996, Annals of Medicine 28: 499-510; Gibson, 1996, Cancer and Metastasis Reviews 15: 287-299) and/or triple helix DNA (Gee, J. E. et al. (1994) In: Huber, B. E. and B. I. Carr, Molecular and Immunologic Approaches, Futura Publishing Co., Mt. Kisco, N.Y.) may be used to modulate the activity, expression or synthesis of a target candidate nucleic acid according to methods familiar to one of skill in the art.

Alternatively, small interfering RNA (siRNA) molecules can also be used to inhibit the expression of nucleic acids for a candidate receptor or for a candidate ligand. RNA interference is a method in which exogenous, short RNA duplexes are administered where one strand corresponds to the coding region of the target mRNA (Elbashir et al., Nature 2001, 411: 494498). Upon entry into cells, siRNA molecules cause not only degradation of the exogenous RNA duplexes, but also of single-stranded RNAs having identical sequences, including endogenous messenger RNAs. Accordingly, siRNA may be more potent and effective than traditional antisense RNA methodologies since the technique is believed to act through a catalytic mechanism.

Preferred siRNA molecules are typically greater than about 19 nucleotides in length and comprise the sequence of a nucleic acid for a candidate receptor or its ligand. Effective strategies for delivering siRNA to target cells include any of the methods described, supra, for delivering antisense nucleic acids. For example, siRNA can be introduced to cells by transduction using physical or chemical transfection. Alternatively siRNAs may be expressed in cells using, e.g., various PolIII promoter expression cassettes that allow transcription of functional siRNA or precursors thereof. See, for example, Scherr et al., Curr. Med. Chem. 2003, 10(3):245-256; Turki et al., Hum. Gene Ther. 2002, 13(18):2197-2201; Cornell et al., Nat. Struct. Biol. 2003, 10(2):91-92.

Pharmaceutical preparations. Compositions used in the therapeutic methods of this invention may be administered (e.g., in vitro or ex vivo to cell cultures, or, more preferably, in vivo to an individual) at therapeutically effective doses to treat a disease or disorder such as OA that is associated with abnormal candidate gene expression and/or activity. For example, compounds, including compounds identified in such screening methods as described above, that bind to a candidate gene or gene product of the invention may be administered to the cells or individual so that expression and/or activity of the candidate gene or gene product is inhibited. The invention therefore also provides pharmaceutical preparations for use, e.g., as therapeutic compounds to treat disorders, including OA, that are associated with abnormal candidate gene expression or activity.

The terms “therapeutically effective dose” and “effective amount” refer to the amount of the compound that is sufficient to result in a therapeutic response. In embodiments where a compound (e.g., a drug or toxin) is administered in a complex (e.g., with a specific antibody), the terms “therapeutically effective dose” and “effective amount” may refer to the amount of the complex that is sufficient to result in a therapeutic response. A therapeutic response may be any response that a user (e.g., a clinician) will recognize as an effective response to the therapy. Thus, a therapeutic response will generally be an amelioration of one or more symptoms of a disease or disorder. In preferred embodiments, where the pharmaceutical preparations are used to treat OA, a therapeutic response may be a reduction in the amount of cartilage degradation observed, e.g., in biopsies from a patient during treatment.

Toxicity and therapeutic efficacy of compounds can be determined by standard pharmaceutical procedures, for example in cell culture assays or using experimental animals to determine the LD₅₀ and the ED₅₀. The parameters LD₅₀ and ED₅₀ are well known in the art, and refer to the doses of a compound that are lethal to 50% of a population and therapeutically effective in 50% of a population, respectively. The dose ratio between toxic and therapeutic effects is referred to as the therapeutic index and may be expressed as the ratio: LD₅₀/ED₅₀. Compounds that exhibit large therapeutic indices are preferred.

While compounds that exhibit toxic side effects may be used, however, in such instances it is particularly preferable to use delivery systems that specifically target such compounds to the site of affected tissue so as to minimize potential damage to other cells, tissues or organs and to reduce side effects.

Data obtained from cell culture assay or animal studies may be used to formulate a range of dosages for use in humans. The dosage of compounds used in therapeutic methods of the present invention preferably lie within a range of circulating concentrations that includes the ED₅₀ concentration but with little or no toxicity (e.g. below the LD₅₀ concentration). The particular dosage used in any application may vary within this range, depending upon factors such as the particular dosage form employed, the route of administration utilized, the conditions of the individual (e.g., patient), and so forth.

A therapeutically effective dose may be initially estimated from cell culture assays and formulated in animal models to achieve a circulating concentration range that includes the IC₅₀. The IC₅₀ concentration of a compound is the concentration that achieves a half-maximal inhibition of symptoms (e.g., as determined from the cell culture assays). Appropriate dosages for use in a particular individual, for example in human patients, may then be more accurately determined using such information.

Measures of compounds in plasma may be routinely measured in an individual such as a patient by techniques such as high performance liquid chromatography (HPLC) or gas chromatography.

Pharmaceutical compositions for use in accordance with the present invention may be formulated in conventional manner using one or more physiologically acceptable carriers or excipients.

Thus, the compounds and their physiologically acceptable salts and solvates may be formulated for administration by inhalation or insufflation (either through the mouth or the nose) or oral, buccal, parenteral or rectal administration.

For oral administration, the pharmaceutical compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

Preparations for oral administration may be suitably formulated to give controlled release of the active compound. For buccal administration the compositions may take the form of tablets or lozenges formulated in conventional manner. For administration by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

The compositions may, if desired, be presented in a pack or dispenser device that may contain one or more unit dosage forms containing the active ingredient. The pack may for example comprise metal or plastic foil, such as a blister pack. The pack or dispenser device may be accompanied by instructions for administration.

Numerous references, including patents, patent applications and various publications, are cited and discussed in the description of this invention. The citation and/or discussion of such references is provided merely to clarify the description of the present invention and is not an admission that any such reference is “prior art” to the invention described herein. All references cited and discussed in this specification (including references to biological sequences deposited in GenBank or other public databases) are incorporated herein by reference in their entirety and to the same extent as if each reference was individually incorporated by reference.

### EXAMPLES

The present invention is also described by means of the following examples. However, the use of these or other examples anywhere in the specification is illustrative only and in no way limits the scope and meaning of the invention or of any exemplified term. Likewise, the invention is not limited to any particular preferred embodiments described herein. Indeed, many modifications and variations of the invention may be apparent to those skilled in the art upon reading this specification and can be made without departing from its spirit and scope. The invention is therefore to be limited only by the terms of the appended claims along with the fall scope of equivalents to which the claims are entitled.

### Example 1

### A High Throughput Screen to Identify Candidate Genes Related to OA Employing RT:PCR Analysis of OA “Marker” Genes

This example describes experiments that use a real time polymerase chain reaction (RT-PCR) assay to identify candidate genes or gene products that may be related to the pathogenesis of OA. In particular, the experiments described in this example test individual full length cDNAs in a high throughput parallel mode for their ability to activate one or more marker genes the expression of which is associated with OA in human articular chondrocyte (HAC) cells.

Materials and Methods:

Data mining OA cDNA libraries. cDNA libraries are preferably generated “in house” from OA chondrocyte cells and used in screening assays of the present invention. Raw sequences of genes in the OA cDNA library are pre-processed and then annotated to identify clones that are likely to be particularly useful as drug targets. In particular, the Phred/Phrap system (Gordon et al., Genome Re. 2001, 11(4):614-625; Ewing et al., Genome Res. 1998, 8:175-185; Ewing et al., Genome Res. 1998, 8:186-194; Gordon et al., Genome Res. 1998, 8;195-202) is used to trim raw sequences to high quality regions and to trim vector sequences. Mitochondrial DNA, ribosomal DNA, repeat regions, low complexity sequence and linker regions are removed. Then, the resulting processed sequences are compared to known and predicted genes in the GenBank database.

Next, the resulting sequence annotations are searched for keywords of interest to select specific clones for screening. The keywords are chosen to emphasize proteins in classes considered most likely to play a role in the disease process based on current biological knowledge. Thus, for example, terms indicative of signal transduction and proteolysis (e.g., “kinase,” “receptor,” “factor” and “protease”) are included since these processes have been previously implicated in osteoarthritis. Individual full length clones for genes selected in this way are then retrieved.

Preparation of plasmid DNA from full length cDNA clones. Bacterial stocks of full-length clones from the OA cDNA libraries in pCMVSport6 vector (Invitrogen, Carlsbad Calif.) are grown in 96 deep-well blocks (Qiagen, Valencia Calif.), each well containing 1.0 mL of Terrific broth (Sigma, St. Louis Mo.) and ampicillin (40 μg/mL). The cultures are initially grown for 24 hours at 37° C. with shaking at 300 RPM, re-innoculated into a fresh block and further grown overnight to ensure uniform growth of bacteria in all wells. Plasmid DNA is isolated from the bacteria with a Biorobot 8000 (Qiagen, Valencia Calif.) following standard protocols described by the manufacturer.

GATEWAY™ transfer of full-length cDNA clones. In order to screen individual clones in an RT-PCR assay, cDNA clones in the OA libraries are transferred from the pCMVSport6 vector to a retroviral vector using the GATEWAY™ platform (Invitrogen, Carlsbad Calif.).

Gateway BP reactions are carried out in 96-well plates (Ashford, United Kingdom). Briefly, 1.0 μL (100-120 ng) plasmid DNA is added to each well containing 1 μL (100-120 ng) pDONR 201 entry vector (Invitrogen, Carlsbad Calif.), 1 μL BP reaction buffer (Invitrogen Carlsbad, Calif.), 1 μL tris-EDTA and 1 μL BP Clonase enzyme mix (Invitrogen, Carlsbad Calif.) on ice. The plates are incubated at 25° C. for three hours.

The Gateway LR reaction mix, consisting of 0.25 μL of 0.75 M NaCl, 1.0 μL (100-120 ng) linearized retroviral vector and 1.5 μL LR Clonase enzyme mix (Invitrogen, Carlsbad Calif.) is added to each BP reaction.

The retroviral vector contains a hybrid cytomegalovirus (CMV)/Maloney murine leukemia virus (MoMuLV) 5′ LTR, a MoMuLV 3′ LTR and a retroviral packaging Ψ site and may be constructed according to conventional methods. The same vector is also commercially available (Clontech). Samples are mixed thoroughly and incubated for two additional hours at 25° C. One-tenth volume (0.8 μL; 2 mg/mL) of Proteinase K solution (Invitrogen, Carlsbad Calif.) is added and incubated at 37° C. for ten minutes.

40 μL of Max efficiency DH5α cells (Invitrogen, Carlsbad Calif.) are aliquoted into wells of a flat bottom 96-well block (Qiagen, Valencia Calif.) on ice. 1 μL of the LR reaction mixture from each well is then added to the cells and incubated on ice for 30 minutes. Cells are heat shocked for 30 seconds at 42° C., placed on ice for 1-2 minutes, and 65 μL of S.O.C. medium (Invitrogen, Carlsbad Calif.) is added to each well. The 96-well block is incubated at 37° C. for one hour with shaking. 35 μL of the final transformation mixture was added to each well of a 2×48 deep-well block containing LB agar with 40 μg/mL zeocin (Invitrogen, Carlsbad Calif.), and was grown overnight at 37° C. Single colonies are inoculated to 1 mL Terrific broth/zeocin (40 μg/mL) in 96-well format and grown overnight at 37° C./300 RPM. Plasmid DNA is isolated using a Biorobot 8000 (Qiagen, Valencia Calif.) following standard protocols described by the manufacturer.

Production of Supernatants. GP2-293 packaging cells (BD Biosciences Clontech, Palo Alto Calif.) are seeded (5×10⁴ cells per well) in 96-well PDL plates (BD Biosciences Clontech, Palo Alto, Calif.) 16-24 hours prior to transfection in antibiotic-free DMEM containing 10% FBS (Invitrogen, Carlsbad Calif.). GATEWAY™ constructs along with envelope vector pVPack-VSV-G (Stratagene, La Jolla Calif.) are cotransfected into the packaging cells by combining 150 ng GATEWAY™ construct with 150 ng envelope plasmid in a total volume of 25 μL OPTIMEM (Invitrogen, Calsbad Calif.) in a 96-well format. In a separate plate, 25 μL of OPTIMEM™ is combined with 1 μL of Lipofectamine 2000 reagent (Invitrogen, Carlsbad Calif.). This second solution is incubated for five minutes at room temperature, and the two solutions are then combined. The DNA-lipofectamine complex is allowed to form for 20 minutes before being added to the cells. The media is replaced with complete media containing antibiotics 16-24 hours after the transfection procedure. The media, containing viral supernatants; is collected at 24 and 48 hours post transfection.

Transduction into Primary Chondrocytes. Primary chondrocytes (isolated from cartilage tissue obtained from joint replacement surgery, Mullenberg Hospital, Plainfield, N.J.) are seeded at 1.1×10⁴ cells perwell in duplicate 96-well plates, twenty-four hours prior to transduction. At time of transduction, media are replaced with 100 μL viral supernatant and 100 μL complete media supplemented with 20 mM HEPES and 16 μg/mL polybrene. Cells are centrifuged in a swinging bucket rotor at 32° C., 1000× g, for 1.5 hours. The media are replaced after 16-24 hours with fresh media, and cells are incubated for an additional 48 hours.

RNA isolation and RT-PCR. Total cellular RNA is isolated from pooled duplicate 96-well plates using a BioRobot 8000 (Qiagen, Valencia Calif.) and Qiagen RNeasy 96 Biorobot reagents according to the manufacturer's instructions. On-column DNase I digestion is employed, pursuant to standard protocols published by Qiagen (Valencia Calif.) to eliminate contaminating genomic DNA. First strand cDNA is synthesized using random primers with a High-Capacity cDNA Archive kit (PE Applied Biosystems, Foster City Calif.) in a 100 μL reaction volume. Real time PCR (RT-PCR) was performed in a 384-well format on the ABI Prism 7900HT Sequence Detection System (Applied Biosystems, Foster City Calif.). The cDNA template and PCR mix are distributed using a Biomek FX liquid handling robot. The 20 μL reaction contains 5 μL cDNA, 200 nM forward and reverse primers, and SYBR Green PCR Master Mix (Applied Biosystems, Foster City Calif.). The default cycling program (95° C.—10 minutes and 40 cycles of 95° C.—15 second, 60° C.—1 minute) is followed by a dissociation stage whereby a melting curve is generated to confirm the specificity of the PCR product and the absence of primer dimers.

Amplification of the ubiquitously expressed gene GAPDH is used to normalize the amount of cDNA added to the reaction. ROX dye is used as a passive reference to normalize non-PCR related fluctuations in the fluorescence signal. Changes in gene expression are calculated according to the manufacturer's instructions using the comparative Cₜ method which makes use of a calibrator sample (i.e., a sample to which all others are compared). The value of the calibrator sample is normalized as 1.0 so that expression levels for all other samples are defined as multiples of the expression level measured for the calibrator sample. For RT-PCR experiments described in this example, a retroviral vector containing no cDNA insert is used as the calibrator sample. Briefly, the amount of target relative to the calibrator is calculated according to the formula: 2^{-ΔΔC}^{ᵣ} where Cₜ=thresh hold cycle (cycle# at which the amount of amplified target reaches a fixed thresh hold).

Cell treatment. To optimize RT-PCR conditions and validate the markers chosen in these screens, human articular chondrocytes from knee joint cartilage obtained in joint replacement surgeries are plated in 96 well plates (11,000 cells per well) using DMEM medium containing 10% FBS (Invitrogen, Carlsbad Calif.). Two days later, the cells are treated with IL-1 (5 ng/mL) (Peprotech, UK, London) and OSM (50 ng/mL) or PDGF (50 ng/mL) or TGF-β (50 ng/mL) overnight in serum free medium. OSM, PDGF and TGF-β are purchased from R&D systems, (Minneapolis, Minn.). RNA is isolated from these cells and evaluated by RT-PCR using the methods described above.

Data Mining for OA associated genes. Early and late OA cDNA libraries are mined to identify the most abundant genes associated with OA cartilage. Among the most highly expressed genes in early OA libraries is C17. An exemplary nucleotide sequence for this gene is available from GenBank Accession No. NM_{—}018659. The C17 gene encodes a protein that has been described as “cytokine-like” and was previously believed to be expressed only in CD34+ hematopoietic cells. The number of ESTs for C17 is higher in early OA than in late OA, suggesting that the expression level of this gene decreases during progression of the disease.

A second abundant gene, known as SMOC2 (available from GenBank Accession No. NM_{—}022138) is highly expressed in late OA cartilage, as evidenced by the higher number of ESTs in a late OA cDNA library than in an early OA cDNA library. Thus, expression of this gene presumably increases during progression of the disease.

OA associated genes are also identified by mining gene expression data generated using DNA microarrays. U95A GeneChips from Affymetrix (Santa Clara, Calif.) are used according to the manufacturer's recommended protocol to compare sets of expressed genes in knee cartilage from 12 OA and 9 healthy patients. The average difference in intensity is calculated for all genes, and the significance of the difference between diseased and healthy patients is evaluated using a statistical t-test. Visual inspection confirms that the computed differences represent differences between patient groups rather than variability in the data. Among the most significantly changed genes between normal and OA knees are the genes OSF-2 (also known as periostin), MARCKS (myristoylated alanine-rich protein kinase C substrate), retinoic acid receptor beta, zinc finger protein Zic1, BASP1 (brain abundant membrane attached signal protein 1), and DIM1. All of these genes are upregulated in OA patients but have not previously been associated with that disease. GenBank Accession numbers for preferred nucleotide sequences sequences of these genes are provided below, along with GenBank Accession numbers for amino acid sequences that are encoded by these nucleic acids.

TABLE I
NEWLY IDENTIFIED OA MARKER GENES

| | GenBank Accession Nos. | | |
|---|---|---|---|
| Gene | Nucleotide | Protein | |
| OSF-2 | NM_006475 | NP_006466 | SEQ ID NO 23 |
| | SEQ ID NO 171 | | |
| MARCKS | NM_002356 | NP_002347 | SEQ ID NO 24 |
| | SEQ ID NO 172 | | |
| Retinoic Acid | NM_00965 | NP_000956 | SEQ ID NO 25 |
| Receptor β | SEQ ID NO 173; | NP_057236 | SEQ ID NO 26 |
| | NM_016152 | | |
| | SEQ ID NO 174 | | |
| BASP1 | NM_006317 | NP_006308 | SEQ ID NO 27 |
| | SEQ ID NO 175 | | |
| Zic1 | NM_003412 | NP_003403 | SEQ ID NO 28 |
| | SEQ ID NO 176 | | |
| DIM1 | NM_006701 | NP_006692 | SEQ ID NO 29 |
| | SEQ ID NO 177 | | |

Choosing OA markers. To identify genes that are involved in osteoarthritis (OA) and/or may be useful for the diagnosis or treatment of that disease, a real time polymerase chain reaction (RT-PCR) based assay is used to screen cDNA clones in a high throughput parallel mode. In particular, the assays described in this example use RT-PCR to measure expression of certain genes that are considered “markers” or indicators of OA.

The marker geness are preferably selected to represent various biological pathways that are affected in OA (see Table II). The GenBank Accession Number for an exemplary nucleotide sequence is also provided for each marker gene. In addition, the gene GAPDH (GenBank Accession No. AJ_{—}005371) is selected as a ubiquitously expressed “housekeeping” gene to which all samples are normalized.

TABLE II
MARKER GENES FOR OA PHENOTYPES

| OA | | |
|---|---|---|
| Pheotype/Characteristic | Marker Gene | Accession No. |
| Cartilage degradation | Aggrecanase-1 | AF148213 |
| | MMP-13 | XM_006274 |
| Aberrant chondrocyte | Collagen Type I | AF017178 |
| cell differentiation | Collagen Type Iia | XM_012271 |
| (hypertrophy and | Collagen Type X | NM_000493 |
| proliferation) | | |
| Inflammation | Inos | AB022318 |
| | Cox-2 | M90100 |
| Matrix synthesis | Aggrecan | X80278 |
| | Decorin | AF91944 |

PCR primers for each of the marker genes is designed with Primer Express software (Applied Biosystems, Foster City Calif.) under default parameters and reaction conditions. The primer sequences used for marker genes in this example are provided in Table III, below.

TABLE III
RT-PCR PRIMERS TO DETECT OA MARKER GENES

| | | |
|---|---|---|
| Marker Gene | Primer | Sequence |

| | | | |
|---|---|---|---|
| Aggrecanase-1 | forward | 5′-TTTCCCTGGCAAGGACTATGA-3′ | (SEQ ID NO:1) |
| | reverse | 5′-AATGGCGTGAGTCGGGC-3′ | (SEQ ID NO:2) |
| MMP-13 | forward | 5′-TGATCTCTTTTGGAATTAAGGAGCAT-3′ | (SEQ ID NO:3) |
| | reverse | 5′-ATGGGCATCTCCTCCATAATTTG-3 | (SEQ ID NO:4) |
| COX-2 | forward | 5′-AAATTGCTGGCAGGGTTGC-3′ | (SEQ ID NO:5) |
| | reverse | 5′-TTTCTGTACTGCGGGTGGAAC-3′ | (SEQ ID NO:6) |
| iNOS | forward | 5′-GGAAACCTTCAAGGCAGCC-3′ | (SEQ ID NO:7) |
| | reverse | 5′-TGCTGTTTGCCTCGGACAT-3′ | (SEQ ID NO:8) |
| Collagen IIa | forward | 5′-ACGCTGCTCGTCGCCG-3′ | (SEQ ID NO:9) |
| | reverse | 5′-GCCAGCCTCCTGGACATCCT-3′ | (SEQ ID NO:10) |
| Collage X | forward | 5′-ACCCAACACCAAGACACAGTTCT-3′ | (SEQ ID NO:11) |
| | reverse | 5′-TCTTACTGCTATACCTTTACTCTTTATGGTGTA-3′ | (SEQ ID NO:12) |
| Collagen I | forward | 5′-CAGCCGCTTCACCTACAGC-3′ | (SEQ ID NO:13) |
| | reverse | 5′-TTTTGTATTCAATCACTGTCTTGCC-3′ | (SEQ ID NO:14) |
| Decorin | forward | 5′-GCCAGCCTCCTGGACATCCT-3′ | (SEQ ID NO:15) |
| | reverse | 5′-AGTCCTTTCAGGCTAGCTGCATC-3′ | (SEQ ID NO:16) |
| Aggrecan | forward | 5′-TCGAGGACAGCGAGGCC-3′ | (SEQ ID NO:17) |
| | reverse | 5′-TCGAGGGTGTAGCGTGTAGAGA-3′ | (SEQ ID NO:18) |
| GAPDH | forward | 5′-ATGGGGAAGGTGAAGGTCG-3′ | (SEQ ID NO:19) |
| | reverse | 5′-TAAAAGCAGCCGTGGTGACC-3′ | (SEQ ID NO:20) |

Expression changes of OA markers. To validate the RT-PCR conditions and primers, human articular chondrocyte cells are treated with various compounds as described in the Materials and Methods section, above, for this example. These compounds are known to induce an OA phenotype in the chondrocyte cells. See, for example, Smith et al., Arthritis Rheum. 1991, 34:697-706; Tardif et al., Arthritis Rheum. 1999, 42:1147-1158.

RT-PCR is performed to determine whether there is any detectable change in expression of one or more marker genes. Table IV, below, summarizes exemplary changes in mRNA levels of each marker mediated by treatment of the chondrocyte cells with: (i) IL-1 and OSM; (ii) TGF-β; and (iii) PDGF. Expression levels are indicated as the multiples of normalized expression levels (i.e., as the “fold changes” in Mrna levels) measured in untreated chondrocyte cells. The data in Table IV indicates that the various OA marker genes undergo the expected changes in their expression levels in response to known treatments that induce an OA phenotype. Moreover, the response of these OA marker genes is sensitive enough to validate this RT-PCR assay for running high throughput functional screens.

TABLE IV
CHANGE OF MARKER GENE EXPRESSION
IN TREATED CHONDROCYTE CELLS

Treatment

| Marker Gene | IL-1/OSM | TGF-β | PDGF | Untreated |
|---|---|---|---|---|
| Aggrecanase-1 | 50.21 | 3.81 | 2.46 | 1.00 |
| MMP-13 | 125.37 | 6.92 | 4.20 | 1.00 |
| Collagen Iia | −227.54 | 1.45 | −2.04 | 1.00 |
| Collagen X | −3.71 | 19.97 | −1.79 | 1.00 |
| Collagen I | −3.58 | 3.84 | −1.89 | 1.00 |

To further validate the RT-PCR assay for use in functional screens, the constitutively active gene AKT/PKB (GenBank Accession No. NPL-001907) is overexpressed in chondrocyte cells by retroviral-mediated gene transfer. Activation of this gene's biochemical pathway induces Aggrecanse-1 and MMP-13 in chondrocyte cells. Cellular RNA is harvested 48 hours and 72 hours post transduction, and changes in the expression of MMP-13 and aggrecanse-1 Mrna are detected by RT-PCR. AKT over-expression results in a 12-fold induction of Aggrecanase-1 and a 9-fold induction of MMP-13.

These experiments validate RT-PCR as a valid and sensitive method that can be used in high throughput functional assays to identify novel mediators of an OA phenotype.

Results:

Verified hits from an RT-PCR screen. The high throughput screen disclosed in this example is performed by overexpressing a select set of about 1200 test genes mined from OA libraries in primary chondrocytes. Expression levels of the OA marker genes are measured by RT-PCR when these test genes are expressed in chondrocyte cells, and these expression levels are compared to the expression levels measured in untransformed cells. To the Applicant's knowledge, heretofore, high throughput screens of chondrocytes have not been reported.

Table V lists 63 candidate genes identified in such an RT-PCR screen, along with GenBank accession numbers for their preferred nucleotide sequences. Residues coding the predicted amino acid sequence (i.e., the coding sequence or “CDS”) are also specified.

TABLE V
CANDIDATE GENES IDENTIFIED IN RT-PCR SCREEN

| | SEQ ID | Accession # | | Accession # |
|---|---|---|---|---|
| Gene | NOS | (nucleotide) | CDS | (protein) |
| SFRS3 | 30/31 | NM_003017 | 106-600 | NP_003008 |
| SFRS10 | 32/33 | NM_004593 | 122-988 | NP_004584 |
| U2AF1 | 34/35 | NM_006758 | 39-761 | NP_006749 |
| TGFBR2 | 36/37 | NM_003242 | 336-2039 | NP_003233 |
| TSC22 | 38/39 | NM_006022 | 192-626 | NP_006013 |
| MTIF3 | 40/41 | NM_152912 | 237-1073 | NP_690876 |
| CAMK2G | 42/43 | XM_044349 | 5-1561 | XP_044349 |
| PHKG1 | 44/45 | NM_006213 | 120-1283 | NP_006204 |
| DTR | 46/47 | NM_001945 | 262-888 | NP_001936 |
| TGFA | 48/49 | NM_003236 | 32-514 | NP_003227 |
| SF3B1 | 50/51 | NM_012433 | 1-3915 | NP_036565 |
| BCAT1 | 52/53 | BC033864 | 424-1386 | AAH33864 |
| CSNK2A1 | 54/55 | NM_001895 | 149-1324 | NP_001886 |
| FLJ14103 | 56/57 | NM_024689 | 76-624 | NP_078965 |
| U5snRNP-AP | 58/59 | AF221842 | 106-2931 | AAF66128 |
| VTI2 | 60/61 | NM_006370 | 341-1039 | NP_006361 |
| LOC51231 | 62/63 | NM_016440 | 119-1543 | NP_057524 |
| TCEA3 | 64/65 | XM_114075 | 136-1182 | XP_114075 |
| UBE2G1 | 66/67 | NM_003342 | 167-679 | NP_003333 |
| SENP3 | 68/69 | NM_015670 | 71-1795 | NP_056485 |
| SF3A3 | 70/71 | NM_006802 | 9-1514 | NP_006793 |
| NRF1 | 72/73 | NM_005011 | 79-1647 | NM_005002 |
| ARF6 | 74/75 | NM_001663 | 518-1045 | NP_001654 |
| TNFSF12 | 76/77 | NM_003809 | 97-846 | NP_003800 |
| RELA | 78/79 | NM_021975 | 39-1652 | NP_068810 |
| TNFRSF1A | 80/81 | NM_001065 | 282-1649 | NP_001056 |
| RPS6KB2 | 82/83 | NM_003952 | 16-1503 | NP_003943 |
| GSK3A | 84/85 | NM_019884 | 115-1566 | NP_063937 |
| CLC | 86/87 | NM_013246 | 46-723 | NP_037378 |
| ZNF213 | 88/89 | XM_036493 | 366-1745 | XP_036493 |
| CAMK1 | 90/91 | NM_003656 | 179-1291 | NP_003647 |
| FGFR1 | 92/93 | NM_023107 | 727-1635 | NP_075595 |
| CLK1 | 94/95 | NM_004071 | 156-1610 | NP_004062 |
| MUS81 | 96/97 | NM_025128 | 511-1941 | NP_079404 |
| VEGF | 98/99 | NM_003376 | 702-1277 | NP_003367 |
| FGF18 | 100/101 | NM_033649 | 538-1161 | NP_387498 |
| HGS | 102/103 | NM_004712 | 78-2411 | NP_004703 |
| RIPK2 | 103/104 | NM_003821 | 212-1834 | NP_003812 |
| TNFAIP1 | 105/106 | NM_021137 | 212-1162 | NP_066960 |
| CLK3 | 107/108 | NM_003992 | 57-1529 | NP_003983 |
| ADAMTS9 | 109/110 | NM_020249 | 13-3231 | NP_064634 |
| CDKN2C | 111/112 | NM_001262 | 1217-1723 | NP_001253 |
| FYN | 113/114 | NM_002037 | 580-2193 | NP_002028 |
| FGF1 | 115/116 | NM_000800 | 142-609 | NP_000791 |
| PTN | 117/118 | NM_002825 | 396-902 | NP_002816 |
| GLA | 119/120 | NM_000169 | 61-1350 | NP_000160 |
| LOC162542 | 121/122 | XM_091624 | 12-287 | XP_091624 |
| EXT2 | 123/124 | NM_000401 | 488-2644 | NP_000392 |
| METAP2 | 125/126 | NM_006838 | 35-1471 | NP_006829 |
| MLL3 | 127/128 | NM_021230 | 364-12441 | NP_067053 |
| RARG | 129/130 | NM_000966 | 138-1502 | NP_000957 |
| Rho GEF p114 | 131/132 | NM_015318 | 108-3155 | NP_056133 |
| CHKL | 133/134 | NM_005198 | 185-1372 | NP_005189 |
| ANXA2 | 135/136 | NM_004039 | 50-1069 | NP_004030 |
| LOC143785 | 137/138 | XM_084635 | 390-1025 | XP_084635 |
| TGFB3 | 139/140 | NM_003239 | 254-1492 | NP_003230 |
| MAP3K11 | 141/142 | NM_002419 | 494-3037 | NP_002410 |
| PHKG2 | 143/144 | NM_000294 | 94-1314 | NP_000285 |
| NNMT | 145/146 | NM_006169 | 118-912 | NP_006160 |
| TPT1 | 147/148 | NM_003295 | 95-613 | NP_003286 |
| IL17BR | 149/150 | NM_018725 | 45-1553 | NP_061195 |
| ECRG4 | 151/152 | NM_032411 | 109-555 | NP_115787 |

### Example 2

### A High Throughput Screen to Identify Candidate Genes Related to OA Employing Analysis of Clonal Proliferation of Chondrocyte Clusters In Vitro

This example describes experiments using another high throughput screen to identify genes and gene products associated with OA. In particular, the experiments described in this example screen whole cDNA libraries and identify genes that induce clonal proliferation of chondrocyte clusters, a type of cell proliferation associated with osteoarthritic chondrocytes.

Materials and Methods:

Construction of late-OA cDNA library. 1 μg of polyA(+) RNA is isolated from 200 μg of total RNA (extracted from OA chondrocyte cells) using a Dynabeads mRNA Purification kit (Dynal, Lake Success N.Y.) following the manufacturer's recommend protocol. The library is constructed using the Superscript Choice System for cDNA Synthesis (Invitrogen Life Technologies, Carlsbad Calif.). The procedure follows the manufacturer's recommended protocol, but with the modifications specifically noted here. A modified oligo d(T)-NotI primer is used to prime the first-strand synthesis reaction. Following second-strand synthesis, adaptor ligation includes the use of EcoRI half-site adapters and Not I restriction digest to allow for the directional cloning of the size fractionated double-stranded cDNA into the entry vector pENTR2B (Invitrogen Life Technologies, Carlsbad Calif.). This vector is constructed to contain GATEWAY™ site-specific recombination sites (attL1 and attL2) flanking the cloned cDNAs and allows the one-step transfer of cDNA inserts into retroviral vectors containing the attR1 and attR2 site-specific recombination sites via LR clonase.

Transfer of Late-OA library. 300 ng of amplified library DNA is used for the transfer of cDNAs into each of two linearized retroviral vectors using LR Clonase (Invitrogen, Carslbad Calif.) according to the manufacturer's recommended protocol. Following a brief clean-up step, the LR reaction products are electroporated into STBL4 electrocompetent cells (Invitrogen Life Technologies, Carlsbad Calif.) and amplified on selective solid medium.

Construction of Early-OA cDNA library. cDNA libraries are constructed “in house” from chondrocytes isolated from early stage human OA cartilage, following the same procedure as for the late-OA cDNA library, above, but with the following exceptions. A modified oligo d(T)-Sfil(B) primer primes the first strand synthesis reaction. Following second-strand synthesis, adaptor ligation includes the use of Sfi I (A) half-site adapters and Sfi I restriction digest to allow for the directional cloning of the size fractionated double-stranded cDNA into the vector pCMBSport6 (Invitrogen Life Technologies, Carlsbad Calif.). This vector has been constructed to contain the GATEWAY™ site-specific recombination sites attB1 and attB2 flanking the cloned cDNAs and requires a two-step transfer of cDNA inserts—first into an entry vector (BP reaction) and second into a retroviral vector containing the attR1 and attR2 site-specific recombination sites via LR cleanse (LR Reaction; Nitrogen, Carlsbad Calif.). The early-OA cDNA library is transferred into a retroviral vector using DH10B cells from Invitrogen (Carlsbad, Calif.) as the host E. coli strain.

Transfection. GP2-293 cells are plated the day before transfection at 7×10⁵ cells per well in 6-well Bio coat plates (BD Biosciences, Palo Alto Calif.) with 2 M DMEM containing 10% FBS per well (Nitrogen, Carlsbad Calif.). The following day, for each well to be transfected, 1 μg of OA cDNA library DNA and 1 μg of pVpack-VSVG plasmids are diluted in OPTIMEM™ medium (Invitrogen, Carlsbad Calif.) to a final volume of 250 μL.

Lipofectamine 2000 (Invitrogen, Carlsbad Calif.) (9 μL/2 μg DNA for each well) is diluted in OPTIMEM™ to 250 μL final volume. The diluted Lipofectamine is added drop wise to the diluted DNA, gently mixed and incubated at room temperature for 20 minutes. The DNA-Lipofectamine complex (500 μL per well) is then added directly into the 2 nL conditioned medium, and the plates are incubated overnight at 37° C. The following day, the medium in each well is aspirated and replaced with 3 mL DMEM containing 10% FBS per well. Supernatants are collected 48 hours and 72 hours post transfection, filtered through a 0.22 micron filter and frozen at −80° C.

Spinfection of viral supernatants into chondrocytes. Human chondrocyte cells (Cell Applications, San Diego Calif.) derived from fetal human cartilage are cryopreserved at the first passage and used at passage 2. The chondrocyte cells are cultured in six well plates at a cell density of 2.5×10⁵ cells per well. The complete growth media is replaced with spinnoculation medium containing DMEM, 10% FBS, 8 μg/mL polybrene and 10 μM HEPES). The viral supernatants are diluted 1:2 with this medium, filtered through a 0.22 micron filter and added to the wells (2 mL/well). The chondrocyte cells are centrifuged for 1.5 hours at 2700 rpm, 32° C. The cells are then placed in a CO₂ incubator for six hours. At the end of the day, 2 mL fresh spinnoculation media is added and the cells are incubated overnight. The next day, the spinnoculation media is replaced with growth media (containing DMEM and 10% FBS), and the cells are cultured for three days.

Chondrocyte cloning assay. Three days post transduction, the chondrocyte cells are trypsinized and suspended in 0.4% low melt agarose (Life Technologies, Rockville Md.) in complete DMEM (Invitrogen, Carlsbad Calif.) at a density of 1×10⁴ cells/mL. 8 mL of the chondrocyte cell suspension is pipeted into 10 cm tissue culture plates that have been previously coated with 8 mL of 0.7% low melt agarose in DMEM containing 10% FBS (Invitrogen, Carlsbad Calif.). The agarose is allowed to solidify at room temperature, and then placed in a 37° C. humidified incubator for 3-4 weeks.

Identification of chondrocyte cell clones. Chondrocyte cell clones are identified using a microscope under 20× magnification, picked using a hand pipetor and the seeded directly into 6-well cluster plates (BD Biosciences Clontech, Palo Alto Calif.) at one clone per well. Clones are allowed to expand in monolayer culture (DMEM, 10% FBS) until confluent.

RNA is isolated using RNeasy 96 (Qiagen, Valencia Calif.). RT-PCR is performed using 96 well format Advantage RT-PCR kit. (Clontech, Palo Alto Calif.) with Amplitaq Gold (Perkin Elmer, Palo Alto Calif.), with the following primers for the AttB sites flanking each cDNA:

| | | |
|---|---|---|
| AttB1 | 5′-CAAGTTTGTACAAAAAAGC-3′ | (SEQ ID NO:21) |
| AttB2 | 5′-ACCACTTTGTACAAGAAAG-3′ | (SEQ ID NO:22) |

The cDNA sequences thus isolated are cloned using a TOPO TA cloning kit (Invitrogen, Carlsbad Calif.). The plasmid DNA is then sequenced by standard sequencing methods (Seqwright, Houston Tex.) for identification. Full length clones corresponding to the identified genes were obtained from a full length clone collection that is generated “in house” by routine methods.

GATEWAY™ transfer of full-length clones. Full length cDNA clones obtained from an in house collection and are transferred into a retroviral vector using the Gateway™ platform as described above, and the fidelity of all clones is verified by nucleotide sequencing (Seqwright, Houston Tex.).

Image Analysis. Validation of a cDNA's ability to promote clonal chondrocyte cell proliferation is measured in agarose cultures of single-gene transduced chondrocyte cells. Chondrocyte cell response is based on the number of clones formed that are greater than 50 microns in diameter. Chondrocyte cell clones are observed using an Olympus IX70 inverted microscope with a 4× objective (Olympus America, Inc.; Melville N.Y.) under brightfield illumination. Each culture dish is photographed at five different microscope fields on three replicate plates and digitally captured on an Olympus MagnaFire camera and software. Each image is then analyzed using Image-Pro Plus v.4.5 software (Media Cybernetics, Inc., (Silver Spring, Md.). Each image is optimized for cell and clone recognition using enhancement filters before being counted. Counts are done automatically using the software, and mean diameter measurements of cells or clones were compiled on an Excel (Microsoft Corporation, Redmond Wash.) spreadsheet.

Results:

Normal chondrocyte cells quickly lose their phenotype and become fibroblastic when grown in monolayer cultures. However, when grown in a 3-dimensional matrix (e.g., of agarose or alginate) these cells remain chondrocytic in their appearance, gene expression profile and low cell division rate. See, Benya & Shaffer, Cell 1982, 30:215-224; Glowacki et al., Proc. Soc. Exp. Biol. and Med. 1983, 172:93-98. Under these culturing conditions, certain growth factors have been shown to induce cell proliferation, as evidenced by the formation of cell clusters reminiscent of the clusters observed in OA cartilage. Kato et al., J. Cell Physiol. 1987, 133:491-498; Iwamoto et al., Biochem. Biophys. Res. Comm. 1989, 159:1006-1011.

To evaluate whether such growth characteristics could be used in a functional screening assay, the clone forming activity of transduced chondrocyte cells overexpressing bFGF is compared to clone forming activity in chondrocyte cells cultured with bFGF exogenously added to the culture medium. The results demonstrate that expression of a retrovirally transduced gene in chondrocyte cells can stimulate cell proliferation in a manner similar to that observed when the gene product is added exogenously (data not shown).

cDNA libraries are constructed from both early and late stage OA cartilage tissue and transferred to retroviral vectors. These libraries can be virally packed and transduced in early passage human chondrocyte cells. Following growth in suspension cultures for 3-4 weeks, cell clusters are isolated using a micropipet under magnification. The transgenes are recovered from these cell clusters using PCR, and are identified by routine nucleotide sequencing. The recovered transgenes are preferably verified by determining whether they induce chondrocyte cluster formation when the full length genes are over expressed individually in chondrocyte cells.

Table VI, below, lists candidate genes that can be identified and verified by such a screening assay. GenBank accession numbers for the preferred nucleotide sequences of those genes are also specified, along with the residues coding the predicted amino acid sequence (i.e., the “CDS”) accession numbers for preferred amino acid sequences of their gene product(s).

TABLE VI
CANDIDATE GENES IDENTIFIED IN CLONAL SCREENS

| | | Accession # | | Accession # |
|---|---|---|---|---|
| Gene | SEQ ID NOs | (nucleotide) | CDS | (protein) |
| C1r | 153/154 | NM_001733 | 52-2169 | NP_001724 |
| NDUFV2 | 155/156 | NM_021074 | 19-768 | NP_066552 |
| BPOZ | 157/158 | NM_032548 | 505-1515 | NP_115937 |
| IL17-RC | 159/160 | NM_032732 | 198-1814 | NP_116121 |
| COMP | 161/162 | NM_000095 | 26-2219 | NP_000086 |
| SLC16A3 | 167/168 | NM_004207 | 1-465 | NP_004198 |
| FGF1 | 169/170 | NM_000800 | 142-609 | NP_000791 |

Candidate genes which may be identified in such clonal screening assays include the bFGF gene, further validating the screening assay.

### EXAMPLE 3

### Sequences for Candidate Genes and Newly Identified OA Marker Genes Identified Herein

>gi|24025684|gb|NM_003017.2|SFRS3 1403 bp mRNA Homo sapiens
splicing factor, arginine/serine-rich 3 (SFRs3), mRNA.
CCGGGTGAGTGAGAGAGTTGGTTGGTGTTGGGCCGGAGGAAAGCGGGAAGACTCATCGGA
GCGTGTGGATTTGAGCCGCCGCATTTTTTAACCCTAGATCTCGAAATGCATCGTGATTCC
TGTCCATTGGACTGTAAGGTTTATGTAGGCAATCTTGGAAACAATGGCAACAAGACGGAA
TTGGAACGGGCTTTTGGCTACTATGGACCACTCCGAAGTGTGTGGGTTGCTAGAAACCCA
CCCGGCTTTGCTTTTGTTGAATTTGAAGATCCCCGAGATGCAGCTGATGCAGTCCGAGAG
CTAGATGGAAGAACACTATGTGGCTGCCGTGTAAGAGTGGAACTGTCGAATGGTGAAAAA
AGAAGTAGAAATCGTGGCCCACCTCCCTCTTGGGGTCGTCGCCCTCGAGATGATTATCGT
AGGAGGAGTCCTCCACCTCGTCGCAGATCTCCAAGAAGGAGAAGCTTCTCTCGCAGCCGG
AGCAGGTCCCTTTCTAGAGATAGGAGAAGAGAGAGATCGCTGTCTCGGGAGAGAAATCAC
AAGCCGTCCCGATCCTTCTCTAGGTCTCGTAGTCGATCTAGGTCAAATGAAAGGAAATAG
AAGACAGTTTGCAAGAGAAGTGGTGTACAGGAAATTACTTCATTTGACAGGAGTATGTAC
AGAAAATTCAAGTTTTGTTTGAGACTTCATAAGCTTGGTGCATTTTTAAGATGTTTTAGC
TGTTCAAATCTGTTTGTCTCTTGAAACAGTGACACAAAGGTGTAATTCTCTATGGTTTGA
AATGGATCATACGAGGCATGTAATACCAAGAATTGTTACTTTACAATGTTCCCTTAAGCA
AAATTGAATTTGCTTTGAACTTTTAGTTATGCACAGACTGATAATAAACCTCTAAACCTG
CCCAGCGGAAGTGTGTTTTTTTTTAAATTTAAATACAGAAACAACTGGCAAAAATTGAAC
TAAGATTTACTTTTTTTTCCATAGCTGGGATATAGGCTGCAGCTATAGTTGAACAAGCAG
TCTTTAAAAACTGCTGTGAAACACAGGCCATCAGGGAAAACGAAATGCTGCACTATTAAA
TTAGAGGTTTTTGAAAAATCCAACTCTCATCCTGGGCAGAGGTTGCCTAGTTGGTATAGA
ATGTTAAGTTTCAAGAAAGTTTACCTTTGCTTTAGGTCATAAGTTCCTTATTTGATTGCT
GTATATGGATACATGGCTGTTCGTGACATTCTTTATGTGCAAATTTGTGATTTCAAAAAT
GTCCTGCCAGTTTAAGGGTACATTGTAGAGCCGAACTTTGAGTTACTGTGCAAGATTTTT
TTTTCATGCTGTCATTTGTAATATGTTTTGTGAGAATCCTTGGGATTAAAGTTTTGGTTA
CAAATTGTTAAAAAAAAAAAAAA
>gi|4506901|gb|NP_003008.1|SFRS3 164 aa linear splicing
factor, arginine/serine-rich 3; splicing factor, arginine//
serine-rich, 20-kD [Homo sapiens].
MHRDSCPLDCKVYVGNLGNNGNKTELERAFGYYGPLRSVWVARNPPGFAFVEFEDPRDAA
DAVRELDGRTLCGCRVRVELSNGEKRSRNRGPPPSWGRRPRDDYRRRSPPPRRRSPRRRS
FSRSRSRSLSRDRRRERSLSRERNRKPSRSFSRSRSRSRSNERK
>gi|4759097|gb|NM_004593.1|SFRS10 1972 bp mRNA Homo sapiens
splicing factor, arginine/serine-rich 10 (transformer 2
homolog, Drosophila) (SFRS10), mRNA.
GAATTCGGCACGAGGGCGACCGGCGCGTCGTGCGGGGCTGCGGCGGAGCCTCCTTAAGGA
AGGTGCAAGAGGTTGGCAGCTTCGATTGAAGCACATCGACCGGCGACAGCAGCCAGGAGT
CATGAGCGACAGCGGCGAGCAGAACTACGGCGAGCGGGAATCCCGTTCTGCTTCCAGAAG
TGGAAGTGCTCACGGATCGGGGAAATCTGCAAGGCATACCCCTGCAAGGTCTCGCTCCAA
GGAAGATTCCAGGCGTTCCAGATCAAAGTCCAGGTCCCGATCTGAATCTAGGTCTAGATC
CAGAAGAAGCTCCCGAAGGCATTATACCCGGTCACGGTCTCGCTCCCGCTCCCATAGACG
ATCACGTAGCAGGTCTTACAGTCGAGATTATCGTAGACGGCACAGCCACAGCCATTCTCC
CATGTCTACTCGCAGGCGTCATGTTGGGAATCGGGCAAATCCTGATCCTAACTGTTGTCT
TGGAGTATTTGGGCTGAGCTTGTACACCACAGAAAGAGATCTAAGAGAAGTGTTCTCTAA
ATATGGTCCCATTGCCGATGTGTCTATTGTATATGACCAGCAGTCTAGGCGTTCAAGAGG
ATTTGCCTTTGTATATTTTGAAAATGTAGATGATGCCAAGGAAGCTAAAGAACGTGCCAA
TGGAATGGAGCTTGATGGGCGTAGGATCAGAGTTGATTTCTCTATAACAAAAAGACCACA
TACGCCAACACCAGGAATTTACATGGGGAGACCTACCTATGGCAGCTCTCGCCGTCGGGA
TTACTATGACAGAGGATATGATCGGGGCTATGATGATCGGGACTACTATAGCAGATCATA
CAGAGGAGGAGGTGGAGGAGGAGGAGGATGGAGAGCTGCCCAAGACAGGGATCAGATTTA
TAGAAGGCGGTCACCTTCTCCTTACTATAGTCGTGGAGGATACAGATCACGTTCCAGATC
TCGATCATACTCACCTCGTCGCTATTAAAGCATGAAGACTTTCTGAAACCTGCCCTAGAG
CTGGGATATTGTTTGTGGGCAATATTTTTTATTGTCTCTTGTTTAAAAAGTGAACAGTGC
CTAGTGAAGTTAGGTGACTTTTACACCTTTTACGATGACTACTTTTGGTGGAGTTGAAAT
GCTGTTTTCATTCTGCATTTGTGTAGTTTGGTGCTTTGTTCCAAGTTAAGTGTTTTCAGA
AAAGTATGTTTTGCATGTATTTTTTTACAGTCTAAATTTTGACTGCTGAGAAGTTTCTAT
TGTACAAAACTTCATTTAAAAGGTTTTTCTACTGAATCCAGGGTATTCTGAAGATCGAAG
CCTGTGTAAAATGCTACCAAATGGCAAAAAGCAACAATAAACAGTTTGATTTTTACTTTT
CTTTCTAACATATCAATGCTTAGCAGAACTATTCAGATTGTCAGTAGTAAATTTAAAGAC
AAATGCCCGTTTTCCTCCAGTCCATGAAACATACCATACTTATATACCTGCAACTAAGTG
TTTAAAATTATGCTCTGTAACTCTGTACTGCTAGTATTAGAACTAAAAATCTTAAAATAC
AGCCAGTGCTTAATGCTTATATCAATGTGGATTTGTCGGCTTTTATGTAATCTGTAATAT
GTATAGCAGGAAATACGAAGAGTTACACAGTGTATGCCTTAAAAGGCTGTTTCTTAAAGG
TGTTACAAGGGGATAATGGTATTTCAACTAGTTATCAGCAAGTGACAATACATTCCACCA
CAAATACACTCTTGTTCTTCTAGCTTTTAGACTATATGAAAAAACCGGGTGCTTCAAAGT
ACATGATAAGGGAACACTATACCTGTCATGGATGAACTGAAGACTTTGCCTGTTCATTTT
TTAAATATTATTTTCAGGTCCTTTGCTTACCAAAGGAGGCCCAATTTCACTCAAATGTTT
TGAGAACTGTGTTTAAATAAACGCAAATGAAAAGAAAAAAAAAAAAAAAAAA
>gi|4759098|gb|NP_004584.1|SFRS10 288 aa linear splicing
factor, arginine/serine-rich 10 (transformer 2 homolog,
Drosophila); splicing factor, arginine/serine-rich
(transformer 2 Drosophila homolog) 10 [Homo sapiens].
MSDSGEQNYGERESRSASRSGSAHGSGKSARHTPARSRSKEDSRRSRSKSRSRSESRSRS
RRSSRRHYTRSRSRSRSHRRSRSRSYSRDYRRRHSHSHSPMSTRRRHVGNRANPDPNCCL
GVFGLSLYTTERDLREVFSKYGPIADVSIVYDQQSRRSRGFAFVYFENVDDAKEAKERAN
GMELDGRRIRVDFSITKRPHTPTPGIYMGRPTYGSSRRRDYYDRGYDRGYDDRDYYSRSY
RGGGGGGGGWRAAQDRDQIYRRRSPSPYYSRGGYRSRSRSRSYSPRRY
>gi|5803206|gb|NM_006758.1|U2AF1 904 bp mRNA Homo sapiens
U2(RNU2) small nuclear RNA auxiliary factor 1 (U2AF1), mRNA.
GGAATTCCGTCGACGGCAGCGGCGGCGGCGGGTGGGAAATGGCGGAGTATCTGGCCTCCA
TCTTCGGCACCGAGAAAGACAAAGTCAACTGTTCATTTTATTTCAAAATTGGAGCATGTC
GTCATGGAGACAGGTGCTCTCGGTTGCACAATAAACCGACGTTTAGCCAGACCATTGCCC
TCTTGAACATTTACCGTAACCCTCAAAACTCTTCCCAGTCTGCTGACGGTTTGCGCTGTG
CCGTGAGCGATGTGGAGATGCAGGAACACTATGATGAGTTTTTTGAGGAGGTTTTTACAG
AAATGGAGGAGAAGTATGGGGAAGTAGAGGAGATGAACGTCTGTGACAACCTGGGAGACC
ACCTGGTGGGGAACGTGTACGTCAAGTTTCGCCGTGAGGAAGATGCGGAAAAGGCTGTGA
TTGACTTGAATAACCGTTGGTTTAATGGACAGCCGATCCACGCCGAGCTGTCACCCGTGA
CGGACTTCAGAGAAGCCTGCTGCCGTCAGTATGAGATGGGAGAATGCACACGAGGCGGCT
TCTGCAACTTCATGCATTTGAAGCCCATTTCCAGAGAGCTGCGGCGGGAGCTGTATGGCC
GCCGTCGCAAGAAGCATAGATCAAGATCCCGATCCCGGGAGCGTCGTTCTCGGTCTAGAG
ACCGTGGTCGTGGCGGTGGCGGTGGCGGTGGTGGAGGTGGCGGCGGACGGGAGCGTGACA
GGAGGCGGTCGAGAGATCGTGAAAGATCTGGGCGATTCTGAGCCATGCCATTTTTACCTT
ATGTCTGCTAGAAAGTGTTGTAGTTGATTGACCAAACCAGTTCATAAGGGGAATTTTTTA
AAAAACAACAAAAAAAAAACATACAAAGATGGGTTTCTGAATAAAAATTTGTAGTGATAA
CAGT
>gi|5803207|gb|NP_006749.1|U2AF1 240 aa linear U2 small
nuclear RNA auxiliary factor 1; U2 snRNP auxiliary factor
small subunit; splicing factor U2AF 35kDa subunit [Homo
sapiens].
MAEYLASIFGTEKDKVNCSFYFKIGACRHGDRCSRLHNKPTFSQTIALLNIYRNPQNSSQ
SADGLRCAVSDVEMQEHYDEFFEEVFTEMEEKYGEVEEMNVCDNLGDHLVGNVYVKFRRE
EDAEKAVIDLNNRWFNGQPIHAELSPVTDFREACCRQYEMGECTRGGFCNFMHLKPISRE
LRRELYGRRRKKHRSRSRSRERRSRSRDRGRGGGGGGGGGGGGRERDRRRSRDRERSGRF
>gi|23308726|gb|NM_003242.3|TGFBR2 2090 bp mRNA Homo sapiens
transforming growth factor, beta receptor II (70/80kDa)
(TGFBR2), mnRNA.
GTTGGCGAGGAGTTTCCTGTTTCCCCCGCAGCGCTGAGTTGAAGTTGAGTGAGTCACTCG
CGCGCACGGAGCGACGACACCCCCGCGCGTGCACCCGCTCGGGACAGGAGCCGGACTCCT
GTGCAGCTTCCCTCGGCCGCCGGGGGCCTCCCCGCGCCTCGCCGGCCTCCAGGCCCCTCC
TGGCTGGCGAGCGGGCGCCACATCTGGCCCGCACATCTGCGCTGCCGGCCCGGCGCGGGG
TCCGGAGAGGGCGCGGCGCGGAGCGCAGCCAGGGGTCCGGGAAGGCGCCGTCCGTGCGCT
GGGGGCTCGGTCTATGACGAGCAGCGGGGTCTGCCATGGGTCGGGGGCTGCTCAGGGGCC
TGTGGCCGCTGCACATCGTCCTGTGGACGCGTATCGCCAGCACGATCCCACCGCACGTTC
AGAAGTCGGTTAATAACGACATGATAGTCACTGACAACAACGGTGCAGTCAAGTTTCCAC
AACTGTGTAAATTTTGTGATGTGAGATTTTCCACCTGTGACAACCAGAAATCCTGCATGA
GCAACTGCAGCATCACCTCCATCTGTGAGAAGCCACAGGAAGTCTGTGTGGCTGTATGGA
GAAAGAATGACGAGAACATAACACTAGAGACAGTTTGCCATGACCCCAAGCTCCCCTACC
ATGACTTTATTCTGGAAGATGCTGCTTCTCCAAAGTGCATTATGAAGGAAAAAAAAAAGC
CTGGTGAGACTTTCTTCATGTGTTCCTGTAGCTCTGATGAGTGCAATGACAACATCATCT
TCTCAGAAGAATATAACACCAGCAATCCTGACTTGTTGCTAGTCATATTTCAAGTGACAG
GCATCAGCCTCCTGCCACCACTGGGAGTTGCCATATCTGTCATCATCATCTTCTACTGCT
ACCGCGTTAACCGGCAGCAGAAGCTGAGTTCAACCTGGGAAACCGGCAAGACGCGGAAGC
TCATGGAGTTCAGCGAGCACTGTGCCATCATCCTGGAAGATGACCGCTCTGACATCAGCT
CCACGTGTGCCAACAACATCAACCACAACACAGAGCTGCTGCCCATTGAGCTGGACACCC
TGGTGGGGAAAGGTCGCTTTGCTGAGGTCTATAAGGCCAAGCTGAAGCAGAACACTTCAG
AGCAGTTTGAGACAGTGGCAGTCAAGATCTTTCCCTATGAGGAGTATGCCTCTTGGAAGA
CAGAGAAGGACATCTTCTCAGACATCAATCTGAAGCATGAGAACATACTCCAGTTCCTGA
CGGCTGAGGAGCGGAAGACGGAGTTGGGGAAACAATACTGGCTGATCACCGCCTTCCACG
CCAAGGGCAACCTACAGGAGTACCTGACGCGGCATGTCATCAGCTGGGAGGACCTGCGCA
AGCTGGGCAGCTCCCTCGCCCGGGGGATTGCTCACCTCCACAGTGATCACACTCCATGTG
GGAGGCCCAAGATGCCCATCGTGCACAGGGACCTCAAGAGCTCCAATATCCTCGTGAAGA
ACGACCTAACCTGCTGCCTGTGTGACTTTGGGCTTTCCCTGCGTCTGGACCCTACTCTGT
CTGTGGATGACCTGGCTAACAGTGGGCAGGTGGGAACTGCAAGATACATGGCTCCAGAAG
TCCTAGAATCCAGGATGAATTTGGAGAATGCTGAGTCCTTCAAGCAGACCGATGTCTACT
CCATGGCTCTGGTGCTCTGGGAAATGACATCTCGCTGTAATGCAGTGGGAGAAGTAAAAG
ATTATGAGCCTCCATTTGGTTCCAAGGTGCGGGAGCACCCCTGTGTCGAAAGCATGAAGG
ACAACGTGTTGAGAGATCGAGGGCGACCAGAAATTCCCAGCTTCTGGCTCAACCACCAGG
GCATCCAGATGGTGTGTGAGACGTTGACTGAGTGCTGGGACCACGACCCAGAGGCCCGTC
TCACAGCCCAGTGTGTGGCAGAACGCTTCAGTGAGCTGGAGCATCTGGACAGGCTCTCGG
GGAGGAGCTGCTCGGAGGAGAAGATTCCTGAAGACGGCTCCCTAAACACTACCAAATAGC
TCTTATGGGGCAGGCTGGGCATGTCCAAAGAGGCTGCCCCTCTCACCAAA
>gi|23308727|gb|NP_003233.3|TGFBR2 567 aa linear trans-
forming growth factor, beta receptor II (70/80kDa); trans-
forming growth factor, beta receptor II (70-80kD) [Homo
sapiens].
MGRGLLRGLWPLHIVLWTRIASTIPPHVQKSVNNDMIVTDNNGAVKFPQLCKFCDVRFST
CDNQKSCMSNCSITSICEKPQEVCVAVWRKNDENITLETVCHDPKLPYHDFILEDAASPK
CIMKEKKKPGETFFMCSCSSDECNDNIIFSEEYNTSNPDLLLVIFQVTGISLLPPLGVAI
SVIIIFYCYRVNRQQKLSSTWETGKTRKLMEFSEHCAIILEDDRSDISSTCANNINHNTE
LLPIELDTLVGKGRFAEVYKAKLKQNTSEQFETVAVKTFPYEEYASWKTEKDIFSDINLK
HENILQFLTAEERKTELGKQYWLITAFHAKGNLQEYLTRHVISWEDLRKLGSSLARGIAH
LRSDHTPCGRPKMPIVHRDLKSSNILVKNDLTCCLCDFGLSLRLDPTLSVDDLANSGQVG
TARYMAPEVLESRMNLENAESFKQTDVYSMALVLWEMTSRCNAVGEVKDYEPPFGSKVRE
HPCVESMKDNVLRDRGRPEIPSFWLNHQGIQMVCETLTECWDHDPEARLTAQCVAERFSE
LEHLDRLSGRSCSEEKIPEDGSLNTTK
>gi|5174728|gb|NM_006022.1|TSC22 1725 bp mRNA Homo sapiens
transforming growth factor beta-stimulated protein TSC-22
(TSC22), mRNA.
CGCCTCTTCACGGCACTGGGATCCGCATCTGCCTGGGATCATCAAGCCCTAGAAGCTGGG
TTTCTTTAAATTAGGGCTGCCGTTTTCTGTTTCTCCCTGGGCTGCGGAAAGCCAGAAGAT
TTTATCTAGCTTATACAAGGCTGCTGGTGTTCCCTCTTTTTTTCCACGAGGGTGTTTTTG
GCTGGAATTGCATGAAATCCCAATGGTGTAGACCAGTGGCGATGGATCTAGGAGTTTACC
AACTGAGACATTTTTCAATTTCTTTCTTGTCATCCTTGCTGGGGACTGAAAACGCTTCTG
TGAGACTTGATAATAGCTCCTCTGGTGCAAGTGTGGTAGCTATTGACAACAAAATCGAGC
AAGCTATGGATCTAGTGAAAAGCCATTTGATGTATGCGGTCAGAGAAGAAGTGGAGGTCC
TCAAAGAGCAAATCAAAGAACTAATAGAGAAAAATTCCCAGCTGGAGCAGGAGAACAATC
TGCTGAAGACACTGGCCAGTCCTGAGCAGCTTGCCCAGTTTCAGGCCCAGCTGCAGACTG
GCTCCCCCCCTGCCACCACCCAGCCACAGGGCACCACACAGCCCCCCGCCCAGCCAGCAT
CGCAGGGCTCAGGACCAACCGCATAGCTGCCTATGCCCCCGCAGAACTGGCTGCTGCGTG
TGAACTGAACAGACGGAGAAGATGTGCTAGGGAGAATCTGCCTCCACAGTCACCCATTTC
ATTGCTCGCTGCGAAAGAGACGTGAGACTGACATATGCCATTATCTCTTTTCCAGTATTA
AACACTCATATGCTTATGGCTTGGAGAAATTTCTTAGTTGGGTGAATTAAAGGTTAATCC
GAGAATTAGCATGGATATACCGGGACCTCATGCAGCTTGGCAGATATCTGAGAAATGGTT
TAATTCATGCTCAGGAGCTGTGTGCCTTTCCATCCCTTCCGGCTCCCTACCCCTCACTTC
CAAGGGTTCTCTCTCCTGCTTGCGCTTAGTGTCCTACATGGGGTTGTGAAGCGATGGAGC
TCCTCACTGGACTCGCCTCTCTCCTCTCCTCCCCCCAGGAGGAACTTGAAAGGAGGGTAA
AAAGACTAAAATGAGGGGGAACAGAGTTCACTGTACAAATTTGACAACTGTCACCAAAAT
TCATAAAAAACAATAGTACTGTGCCTCTTTCTTCTCAAACAATGGATGACACAAAACTAT
GAGAGTGACAAAATGGTGACAGGTAGCTGGGACCTAGGCTATCTTACCATGAAGGTTGTT
TTGCTTATTGTATATTTGTGTATGTAGTGTAACTATTTTGTACAATAGAGGACTGTAACT
ACTATTTAGGTTGTACAGATTGAAATTTAGTTGTTTCATTGGCTGTCTGAGGAGGTGTGG
ACTTTTATATATAGATCTACATAAAAACTGCTACATGACAAAAACCACACCTAAACCCCT
TTTAAGAATTTGGCACAGTTACTCACTTTGTGTAATCTGAAATCTAGCTGCTGAATACGC
TGAAGTAAATCCTTGTTCACTGAAGTCTTTCAATTGAGCTGGTTGAATACTTTGAAAAAT
GCTCAGTTCTAACTAATGAAATGGATTTCCCAGTAGGGGTTTCTGCATATCACCTGTATA
GTAGTTATATGCATATGTTTCTGTGCATGTTCTCTACACAATTGTAAGGTGTCACTGTAT
TTAACTGTTGCACTTGTCAACTTTCAATAAAGCATATAAATGTTG
>gi|5174729|gb|NP_006013.1|TSC22 144 aa linear transforming
growth factor beta-stimulated protein TSC-22 [Homo sapiens]
MKSQWCRPVAMDLGVYQLRHFSISFLSSLLGTENASVRLDNSSSGASVVAIDNKIEQAMD
LVKSHLMYAVREEVEVLKEQIKELIEKNSQLEQENNLLKTLASPEQLAQFQAQLQTGSPP
ATTQPQGTTQPPAQPASQGSGPTA
>gi|24432096|gb|NM_152912.2|MTIF3 1693 bp mRNA Homo sapiens
mitochondrial translational initiation factor 3 (MTIF3),
mRNA.
GCAGATCCGCTGTACTTGCGGGCGCTACAGTATGTCAATCGCTTGCCCCAGCACAGTGGG
CTCCGTGGCTTAAGACTTGAACCAAGTAAACGAAGTTCTCTTACTGAGAAGTCTCAGTTT
CAAAAGAGCTTCTCCTCATCAACTGGGGATGATTACAGTTCTTCCTAAAAAAGCCTACTT
GATGTGAAGACAATGAGGATGAAGACCTTTATGGTGATCCACTTCCACTTAATAGGATGG
CTGCTCTTTTTCTAAAGAGGTTAACACTACAAACTGTAAAGTCTGAAAATAGTTGCATTA
GATGTTTTGGTAAACACATCCTGCAAAAGACAGCACCAGCACAGTTGTCCCCTATTGCTT
CTGCCCCAAGACTCTCCTTCCTAATTCATGCAAAAGCCTTTAGTACCGCTGAAGACACCC
AGAATGAAGGAAAAAAGACAAAAAAGAATAAAACAGCTTTTAGTAACGTTGGAAGAAAAA
TTAGTCAGCGAGTTATTCACTTATTTGATGAGAAGGGCAATGATTTGGGAAACATGCACC
GAGCAAATGTGATTAGACTTATGGATGAGCGAGACCTGCGACTGGTTCAAAGGAACACCA
GCACAGAACCTGCAGAGTATCAGCTCATGACAGGATTGCAGATCCTCCAGGAGCGGCAGA
GGCTGAGGGAGATGGAGAAGGCGAACCCCAAAACTGGACCAACCCTGAGAAAGGAACTGA
TTTTGTCTTCAAATATTGGACAACATGATTTGGACACAAAGACTAAACAGATTCAGCAGT
GGATTAAGAAAAAACACCTAGTCCAGATTACCATAAAGAAAGGAAAAAATGTAGACGTGT
CAGAAAATGAAATGGAGGAGATATTTCATCAAATACTCCAGACTATGCCTGGAATAGCTA
CATTCTCATCTAGGCCACAAGCTGTTCAAGGAGGAAAAGCTTTAATGTGTGTTCTTCGTG
CTTTGAGCAAAAATGAGGAGAAGGCATATAAAGAAACTCAAGAGACCCAGGAAAGAGACA
CTTTGAACAAAGATCATGGAAATGATAAGGAATCAAATGTTCTGCATCAGTAATTTTAAT
AAAGAAAAGCATGCTCTGAGAGAAAAAAAAGCTCGCTCCTTGGTCTGCAGTCCTTTAAAC
AAAGCAGTGCAGTTCTTAGCCAAGGGTAAGTACTGCAACTGTCGAGAGCATCTTGTCTTC
CACACAGTTGGGTGACTCTCCGTTTTGACACAAAGATAAGCCTTGCCCTTGTTTCCTTTT
GGGAGGGATATATCCACTGAGATGAGAGGCCAAACTCCGTTTTTCACGAGATTTTTTGAC
TTTGAGCTTCATTTTCTTCTTGTCAGGATCATGTACAACAGCATGCCTAGTGAGACTTTG
TTTCATTGCAAATGTTTTGCCACAGCCAGCATGTTCACACACAAAAGGGCGGCTTTCCTC
ATGGAAGGAGAGGATATGGCTTTGGAGATTAAACACAGTTGTATAGGTTCTTCCACAGCC
TTCTCTTGGACAGCGACATAATCCCTTCTGGGGCATGAGTTTATGTGTTGCTTAAGGAAC
TTGCGTTAAAGTTTTCCGGCAACTTCACATGGATTCCTTTGAATGAGTTCAAATGTTCCC
ATGCTAAGCTGAGTCTGTGCCATAGCAAACCATGATATAGCAAGTCTCCAGAATGTGTAC
GAATCAATACTCC
>gi|23097266|gb|NP_690876.1|MTIF3 278 aa linear mitochon-
drial translational initiation factor 3 [Homo sapiens].
MAALFLKRLTLQTVKSENSCIRCFGKHILQKTAPAQLSPIASAPRLSFLIHAKAFSTAED
TQNEGKKTKKNKTAFSNVGRKISQRVIHLFDEKGNDLGNMHRA1NIRLMDERDLRLVQRN
TSTEPAEYQLMTGLQILQERQRLREMEKANPKTGPTLRKELILSSNIGQHDLDTKTKQIQ
QWIKKKHLVQITIKKGKNVDVSENEMEEIFHQILQTMPGIATFSSRPQAVQGGKALMCVL
RALSKNEEKAYKETQETQERDTLNKDHGNDKESNVLHQ
>gi|27499034|gb|XM_044349.7|CAMK2G 1776 bp mRNA Homo sapiens
calcium/calmodulin-dependent protein kinase (CaM kinase) II
gamma (CAMK2G), mRNA.
CAGCATGGCCACCACCGCCACCTGCACCCGTTTCACCGACGACTACCAGCTCTTCGAGGA
GCTTGGCAAGGGTGCTTTCTCTGTGGTCCGCAGGTGTGTGAAGAAAACCTCCACGCAGGA
GTACGCAGCAAAAATCATCAATACCAAGAAGTTGTCTGCCCGGGATCACCAGAAACTAGA
ACGTGAGGCTCGGATATGTCGACTTCTGAAACATCCAAACATCGTGCGCCTCCATGACAG
TATTTCTGAAGAAGGGTTTCACTACCTCGTGTTTGACCTTGTTACCGGCGGGGAGCTGTT
TGAAGACATTGTGGCCAGAGAGTACTACAGTGAAGCAGATGCCAGCCACTGTATACATCA
GATTCTGGAGAGTGTTAACCACATCCACCAGCATGACATCGTCCACAGGGACCTGAAGCC
TGAGAACCTGCTGCTGGCGAGTAAATGCAAGGGTGCCGCCGTCAAGCTGGCTGATTTTGG
CCTAGCCATCGAAGTACAGGGAGAGCAGCAGGCTTGGTTTGGTTTTGCTGGCACCCCAGG
TTACTTGTCCCCTGAGGTCTTGAGGAAAGATCCCTATGGAAAACCTGTGGATATCTGGGC
CTGCGGGGTCATCCTGTATATCCTCCTGGTGGGCTATCCTCCCTTCTGGGATGAGGATCA
GCACAAGCTGTATCAGCAGATCAAGGCTGGAGCCTATGATTTCCCATCACCAGAATGGGA
CACGGTAACTCCTGAAGCCAAGAACTTGATCAACCAGATGCTGACCATAAACCCAGCAAA
GCGCATCACGGCTGACCAGGCTCTCAAGCACCCGTGGGTCTGTCAACGATCCACGGTGGC
ATCCATGATGCATCGTCAGGAGACTGTGGAGTGTTTGCGCAAGTTCAATGCCCGGAGAAA
ACTGAAGGGTGCCATCCTCACGACCATGCTTGTCTCCAGGAACTTCTCAGCTGCCAAAAG
CCTATTGAACAAGAAGTCGGATGGCGGTGTCAAGCCACAGAGCAACAACAAAAACAGTCT
CGTAAGCCCAGCCCAAGAGCCCGCGCCCTTGCAGACGGCCATGGAGCCACAAACCACTGT
GGTACACAACGCTACAGATGGGATCAAGGGCTCCACAGAGAGCTGCAACACCACCACAGA
AGATGAGGACCTCAAAGTGCGAAAACAGGAGATCATTAAGATTACAGAACAGCTGATTGA
AGCCATCAACAATGGGGACTTTGAGGCCTACACGAAGATTTGTGATCCAGGCCTCACTTC
CTTTGAGCCTGAGGCCCTTGGTAACCTCGTGGAGGGGATGGATTTCCATAAGTTTTACTT
TGAGAATCTCCTGTCCAAGAACAGCAAGCCTATCCATACCACCATCCTAAACCCACACGT
CCACGTGATTGGGGAGGACGCAGCGTGCATCGCCTACATCCGCCTCACCCAGTACATCGA
CGGGCAGGGTCGGCCTCGCACCAGCCAGTCAGAAGAGACCCGGGTCTGGCACCGTCGGGA
TGGCAAGTGGCTCAATGTCCACTATCACTGCTCAGGGGCCCCTGCCGCACCGCTGCAGTG
AGCTCAGCCACAGGGGCTTTAGGAGATTCCAGCCGGAGGTCCAACCTTCGCAGCCAGTGG
CTCTGGAGGGCCTGAGTGACAGCGGCAGTCCTGTTTGTTTGAGGTTTAAAACAATTCAAT
TACAAAAGCGGCAGCAGCCAATGCACGCCCCTGCATGCAGCCCTCCCGCCCGCCCTTCGT
GTCTGTCTCTGCTGTACCGAGGTGTTTTTTACATTT
>gi|27499035|gb|XP_044349.7|CAMK2G 518 aa linear similar to
calcium/calmodulin-dependent protein kinase II gamma [Mus
musculus] [Homo sapiens].
MATTATCTRFTDDYQLFEELGKGAFSVVRRCVKKTSTQEYAAKIINTKKLSARDHQKLER
EARICRLLKHPNIVRLHDSISEEGFHYLVFDLVTGGELFEDIVAREYYSEADASHCIHQI
LESVNHIHQHDIVHRDLKPENLLLASKCKGAAVKLADFGLAIEVQGEQQAWFGFAGTPGY
LSPEVLRKDPYGKPVDIWACGVILYILLVGYPPFWDEDQHKLYQQIKAGAYDFPSPEWDT
VTPEAKNLINQMLTINPAKRITADQALKHPWVCQRSTVASMMHRQETVECLRKFNARRKL
KGAILTTMLVSRNFSAAKSLLNKKSDGGVKPQSNNKNSLVSPAQEPAPLQTAMEPQTTVV
HNATDGIKGSTESCNTTTEDEDLKVRKQEIIKITEQLIEAINNGDFEAYTKICDPGLTSF
EPEALGNLVEGMDFHKFYFENLLSKNSKPIHTTILNPHVHVIGEDAACIAYIRLTQYIDG
QGRPRTSQSEETRVWHRRDGKWLNVHYHCSGAPAAPLQ
>gi|5453881|gb|NM_006213.1|PHKG1 1377 bp mRNA Homo sapiens
phosphorylase kinase, gamma 1 (muscle) (PHKG1), mRNA.
GGCCTTCAGCCCTCTGTGGTCCCCTCTCCCCGGGGGGCTTTGGGATTCTTGTCAAGCTCC
TTCAAGAGCCTGCAAGCACTTAACCAGCCACCCAGAGTTCCCTCACTGAAGATCTGAGCA
TGACCCGGGACGAGGCACTGCCGGACTCTCATTCTGCACAGGACTTCTATGAGAATTATG
AGCCCAAAGAGATCCTGGGCAGGGGCGTTAGCAGTGTGGTCAGGCGATGCATCCACAAGC
CCACGAGCCAGGAGTACGCCGTGAAGGTCATCGACGTCACCGGTGGAGGCAGCTTGAGCC
CGGAGGAGGTGCGGGAGCTGCGAGAAGCCACGCTGAAGGAGGTGGACATCCTGCGCAAGG
TCTCAGGGCACCCCAACATCATACAGCTGAAGGACACTTATGAGACCAACACTTTCTTCT
TCTTGGTGTTTGACCTGATGAAGAGAGGGGAGCTCTTTGACTACCTCACTGAGAAGGTCA
CCTTGAGTGAGAAGGAAACCAGAAAGATCATGCGAGCTCTGCTGGAGGTGATCTGCACCT
TGCACAAACTCAACATCGTGCACCGGGACCTGAAGCCCGAGAACATTCTCTTGGATGACA
ACATGAACATCAAGCTCACAGACTTTGGCTTTTCCTGCCAGCTGGAGCCGGGAGAGAGGC
TGCGAGAGGTCTGCGGGACCCCCAGTTACCTGGCCCCTGAGATTATCGAGTGCTCCATGA
ATGAGGACCACCCGGGCTACGGGAAAGAGGTGGACATGTGGAGCACTGGCGTCATCATGT
ACACGCTGCTGGCCGGCTCCCCGCCCTTCTGGCACCGGAAGCAGATGCTGATGCTGAGGA
TGATCATGAGCGGCAACTACCAGTTTGGCTCGCCCGAGTGGGATGATTACTCGGACACCG
TGAAGGACCTGGTCTCCCGATTCCTGGTGGTGCAACCCCAGAACCGCTACACAGCGGAAG
AGGCCTTGGCACACCCCTTCTTCCAGCAGTACTTGGTGGAGGAAGTGCGGCACTTCAGCC
CCCGGGGGAAGTTCAAGGTGATCGCTCTGACCGTGCTGGCTTCAGTGCGGATCTACTACC
AGTACCGCCGGGTGAAGCCTGTGACCCGGGAGATCGTCATCCGAGACCCCTATGCCCTCC
GGCCTCTGCGCCGGCTCATCGACGCCTACGCTTTCCGAATCTATGGCCACTGGGTGAAGA
AGGGGCAGCAGCAGAACCGGGCAGCCCTTTTCGAGAACACACCCAAGGCCGTGCTCCTCT
CCCTGGCCGAGGAGGACTACTGAGGGGCTGGCCAGTCAGGGAGGGCTAGGGGGCAGGTGG
GGAGGGGAAGCCATGGAAATACAAGTCAAAGGGGTAAAAAAAAAAAAAAAAAAAAAA
>gi|5453882|gb|NP_006204.1|PHKG1|387 aa linear phosphory-
lase kinase, gamma 1 (muscle) [Homo sapiens]
MTRDEALPDSHSAQDFYENYEPKEILGRGVSSVVRRCIHKPTSQEYAVKVIDVTGGGSFS
PEEVRELREATLKEVDILRKVSGHPNIIQLKDTYETNTFFFLVFDLMKPGELFDYLTEKV
TLSEKETRKIMRALLEVICTLHKLNIVHRDLKPENILLDDNMNIKLTDFGFSCQLEPGER
LREVCGTPSYLAPEIIECSMNEDHPGYGKEVDMWSTGVIMYTLLAGSPPFWHRKQMLMLR
MIMSGNYQFGSPEWDDYSDTVKDLVSRFLVVQPQNRYTAEEALAHPFFQQYLVEEVRHFS
PRGKFKVIALTVLASVRIYYQYRRVKPVTREIVIRDPYALRPLRRLIDAYAFRIYGHWVK
KGQQQNRAALFENTPKAVLLSLAEEDY
>gi|4503412|gb|NM_001945.1|DTR 2360 bp mRNA Homo sapiens
diphtheria toxin receptor (heparin-binding epidermal growth
factor-like growth factor). (DTR), mRNA.
GCTACGCGGGCCACGCTGCTGGCTGGCCTGACCTAGGCGCGCGGGGTCGGGCGGCCGCGC
GGGCGGGCTGAGTGAGCAAGACAAGACACTCAAGAAGAGCGAGCTGCGCCTGGGTCCCGG
CCAGGCTTGCACGCAGAGGCGGGCGGCAGACGGTGCCCGGCGGAATCTCCTGAGCTCCGC
CGCCCAGCTCTGGTGCCAGCGCCCAGTGGCCGCCGCTTCGAAAGTGACTGGTGCCTCGCC
GCCTCCTCTCGGTGCGGGACCATGAAGCTGCTGCCGTCGGTGGTGCTGAAGCTCTTTCTG
GCTGCAGTTCTCTCGGCACTGGTGACTGGCGAGAGCCTGGAGCGGCTTCGGAGAGGGCTA
GCTGCTGGAACCAGCAACCCGGACCCTCCCACTGTATCCACGGACCAGCTGCTACCCCTA
GGAGGCGGCCGGGACCGGAAAGTCCGTGACTTGCAAGAGGCAGATCTGGACCTTTTGAGA
GTCACTTTATCCTCCAAGCCACAAGCACTGGCCACACCAAACAAGGAGGAGCACGGGAAA
AGAAAGAAGAAAGGCAAGGGGCTAGGGAAGAAGAGGGACCCATGTCTTCGGAAATACAAG
GACTTCTGCATCCATGGAGAATGCAAATATGTGAAGGAGCTCCGGGCTCCCTCCTGCATC
TGCCACCCGGGTTACCATGGAGAGAGGTGTCATGGGCTGAGCCTCCCAGTGGAAAATCGC
TTATATACCTATGACCACACAACCATCCTGGCCGTGGTGGCTGTGGTGCTGTCATCTGTC
TGTCTGCTGGTCATCGTGGGGCTTCTCATGTTTAGGTACCATAGGAGAGGAGGTTATGAT
GTGGAAAATGAAGAGAAAGTGAAGTTGGGCATGACTAATTCCCACTGAGAGAGACTTGTG
CTCAAGGAATCGGCTGGGGACTGCTACCTCTGAGAAGACACAAGGTGATTTCAGACTGCA
GAGGGGAAAGACTTCCATCTAGTCACAAAGACTCCTTCGTCCCCAGTTGCCGTCTAGGAT
TGGGCCTCCCATAATTGCTTTGCCAAAATACCAGAGCCTTCAAGTGCCAAACAGAGTATG
TCCGATGGTATCTGGGTAAGAAGAAAGCAAAAGCAAGGGACCTTCATGCCCTTCTGATTC
CCCTCCACCAAACCCCACTTCCCCTCATAAGTTTGTTTAAACACTTATCTTCTGGATTAG
AATGCCGGTTAAATTCCATATGCTCCAGGATCTTTGACTGAAAAAAAAAAAGAAGAAGAA
GAAGGAGAGCAAGAAGGAAAGATTTGTGAACTGGAAGAAAGCAACAAAGATTGAGAAGCC
ATGTACTCAAGTACCACCAAGGGATCTGCCATTGGGACCCTCCAGTGCTGGATTTGATGA
GTTAACTGTGAAATACCACAAGCCTGAGAACTGAATTTTGGGACTTCTACCCAGATGGAA
AAATAACAACTATTTTTGTTGTTGTTGTTTGTAAATGCCTCTTAAATTATATATTTATTT
TATTCTATGTATGTTAATTTATTTAGTTTTTAACAATCTAACAATAATATTTCAAGTGCC
TAGACTGTTACTTTGGCAATTTCCTGGCCCTCCACTCCTCATCCCCACAATCTGGCTTAG
TGCCACCCACCTTTGCCACAAAGCTAGGATGGTTCTGTGACCCATCTGTAGTAATTTATT
GTCTGTCTACATTTCTGCAGATCTTCCGTGGTCAGAGTGCCACTGCGGGAGCTCTGTATG
GTCAGGATGTAGGGGTTAACTTGGTCAGAGCCACTCTATGAGTTGGACTTCAGTCTTGCC
TAGGCGATTTTGTCTACCATTTGTGTTTTGAAAGCCCAAGGTGCTGATGTCAAAGTGTAA
CAGATATCAGTGTCTCCCCGTGTCCTCTCCCTGCCAAGTCTCAGAAGAGGTTGGGCTTCC
ATGCCTGTAGCTTTCCTGGTCCCTCACCCCCATGGCCCCAGGCCACAGCGTGGGAACTCA
CTTTCCCTTGTGTCAAGACATTTCTCTAACTCCTGCCATTCTTCTGGTGCTACTCCATGC
AGGGGTCAGTGCAGCAGAGGACAGTCTGGAGAAGGTATTAGCAAAGCAAAAGGCTGAGAA
GGAACAGGGAACATTGGAGCTGACTGTTCTTGGTAACTGATTACCTGCCAATTGCTACCG
AGAAGGTTGGAGGTGGGGAAGGCTTTGTATAATCCCACCCACCTCACCAAAACGATGAAG
GTATGCTGTCATGGTCCTTTCTGGAAGTTTCTGGTGCCATTTCTGAACTGTTACAACTTG
TATTTCCAAACCTGGTTCATATTTATACTTTGCAATCCAAATAAAGATAACCCTTATTCC
ATAAAAAAAAAAAAAAAAAA
>gi|4503413|gb|NP_001936.1|DTR 208 aa linear diphtheria
toxin receptor (heparin-binding epidermal growth factor-like
growth factor); Diphtheria toxin receptor (heparin-binding
EGF-like growth factor) [Homo sapiens].
MKLLPSVVLKLFLAAVLSALVTGESLERLRRGLAAGTSNPDPPTVSTDQLLPLGGGRDRK
VRDLQEADLDLLRVTLSSKPQALATPNKEEHGKRKKKGKGLGKKRDPCLRKYKDFCIHGE
CKYVKELRAPSCICHPGYHGERCHGLSLPVENRLYTYDHTTILAVVAVVLSSVCLLVIVG
LLMFRYHRRGGYDVENEEKVKLGMTNSH
>gi|4507460|gb|NM_003236.1|TGFA 4119 bp mRNA Homo sapiens
transforming growth factor, alpha (TGFA), mRNA.
CTGGAGAGCCTGCTGCCCGCCCGCCCGTAAAATGGTCCCCTCGGCTGGACAGCTCGCCCT
GTTCGCTCTGGGTATTGTGTTGGCTGCGTGCCAGGCCTTGGAGAACAGCACGTCCCCGCT
GAGTGCAGACCCGCCCGTGGCTGCAGCAGTGGTGTCCCATTTTAATGACTGCCCAGATTC
CCACACTCAGTTCTGCTTCCATGGAACCTGCAGGTTTTTGGTGCAGGAGGACAAGCCAGC
ATGTGTCTGCCATTCTGGGTACGTTGGTGCACGCTGTGAGCATGCGGACCTCCTGGCCGT
GGTGGCTGCCAGCCAGAAGAAGCAGGCCATCACCGCCTTGGTGGTGGTCTCCATCGTGGC
CCTGGCTGTCCTTATCATCACATGTGTGCTGATACACTGCTGCCAGGTCCGAAAACACTG
TGAGTGGTGCCGGGCCCTCATCTGCCGGCACGAGAAGCCCAGCGCCCTCCTGAAGGGAAG
AACCGCTTGCTGCCACTCAGAAACAGTGGTCTGAAGAGCCCAGAGGAGGAGTTTGGCCAG
GTGGACTGTGGCAGATCAATAAAGAAAGGCTTCTTCAGGACAGCACTGCCAGAGATGCCT
GGGTGTGCCACAGACCTTCCTACTTGGCCTGTAATCACCTGTGCAGCCTTTTGTGGGCCT
TCAAAACTCTGTCAAGAACTCCGTCTGCTTGGGGTTATTCAGTGTGACCTAGAGAAGAAA
TCAGCGGACCACGATTTCAAGACTTGTTAAAAAAGAACTGCAAAGAGACGGACTCCTGTT
CACCTAGGTGAGGTGTGTGCAGCAGTTGGTGTCTGAGTCCACATGTGTGCAGTTGTCTTC
TGCCAGCCATGGATTCCAGGCTATATATTTCTTTTTAATGGGCCACCTCCCCACAACAGA
ATTCTGCCCAACACAGGAGATTTCTATAGTTATTGTTTTCTGTCATTTGCCTACTGGGGA
AGAAAGTGAAGGAGGGGAAACTGTTTAATATCACATGAAGACCCTAGCTTTAAGAGAAGC
TGTATCCTCTAACCACGAGACTCTCAACCAGCCCAACATCTTCCATGGACACATGACATT
GAAGACCATCCCAAGCTATCGCCACCCTTGGAGATGATGTCTTATTTATTAGATGGATAA
TGGTTTTATTTTTAATCTCTTAAGTCAATGTAAAAAGTATAAAACCCCTTCAGACTTCTA
CATTAATGATGTATGTGTTGCTGACTGAAAAGCTATACTGATTAGAAATGTCTGGCCTCT
TCAAGACAGCTAAGGCTTGGGAAAAGTCTTCCAGGGTGCGGAGATGGAACCAGAGGCTGG
GTTACTGGTAGGAATAAAGGTAGGGGTTCAGAAATGGTGCCATTGAAGCCACAAAGCCGG
TAAATGCCTCAATACGTTCTGGGAGAAAACTTAGCAAATCCATCAGCAGGGATCTGTCCC
CTCTGTTGGGGAGAGAGGAAGAGTGTGTGTGTCTACACAGGATAAACCCAATACATATTG
TACTGCTCAGTGATTAAATGGGTTCACTTCCTCGTGAGCCCTCGGTAAGTATGTTTAGAA
ATAGAACATTAGCCACGAGCCATAGGCATTTCAGGCCAAATCCATGAAAGGGGGACCAGT
CATTTATTTTCCATTTTGTTGCTTGGTTGGTTTGTTGCTTTATTTTTAAAAGGAGAAGTT
TAACTTTGCTATTTATTTTCGAGCACTAGGAAAACTATTCCAGTAATTTTTTTTTCCTCA
TTTCCATTCAGGATGCCGGCTTTATTAACAAAAACTCTAACAAGTCACCTCCACTATGTG
GGTCTTCCTTTCCCCTCAAGAGAAGGAGCAATTGTTCCCCTGACATCTGGGTCCATCTGA
CCCATGGGGCCTGCCTGTGAGAAACAGTGGGTCCCTTCAAATACATAGTGGATAGCTCAT
CCCTAGGAATTTTCATTAAAATTTGGAAACAGAGTAATGAAGAAATAATATATAAACTCC
TTATGTGAGGAAATGCTACTAATATCTGAAAAGTGAAAGATTTCTATGTATTAACTCTTA
AGTGCACCTAGCTTATTACATCGTGAAAGGTACATTTAAAATATGTTAAATTGGCTTGAA
ATTTTCAGAGAATTTTGTCTTCCCCTAATTCTTCTTCCTTGGTCTGGAAGAACAATTTCT
ATGAATTTTCTCTTTATTTTTTTTTTATAATTCAGACAATTCTATGACCCGTGTCTTCAT
TTTTGGCACTCTTATTTAACAATGCCACACCTGAAGCACTTGGATCTGTTCAGAGCTGAC
CCCCTAGCAACGTAGTTGACACAGCTCCAGGTTTTTAAATTACTAAAATAAGTTCAAGTT
TACATCCCTTGGGCCAGATATGTGGGTTGAGGCTTGACTGTAGCATCCTGCTTAGAGACC
AATCAATGGACACTGGTTTTTAGACCTCTATCAATCAGTAGTTAGCATCCAAGAGACTTT
GCAGAGGCGTAGGATGAAGGCTGGACAGATGGCGGAACGAGAGGTTCCCTGCGAAGACTT
GAGATTTAGTGTCTGTGAATGTTCTAGTTCCTAGGTCCAGCAAGTCACACCTGCCAGTGC
CCTCATCCTTATGCCTGTAACACACATGCAGTGAGAGGCCTCACATATACGCCTCCCTAG
AAGTGCCTTCCAAGTCAGTCCTTTGGAAACCAGCAGGTCTGAAAAAGAGGCTGCATCAAT
GCAAGCCTGGTTGGACCATTGTCCATGCCTCAGGATAGAACAGCCTGGCTTATTTGGGGA
TTTTTCTTCTAGAAATCAAATGACTGATAAGCATTGGCTCCCTCTGCCATTTAATGGCAA
TGGTAGTCTTTGGTTAGCTGCAAAAATACTCCATTTCAAGTTAAAAATGCATCTTCTAAT
CCATCTCTGCAAGCTCCCTGTGTTTCCTTGCCCTTTAGAAAATGAATTGTTCACTACAAT
TAGAGAATCATTTAACATCCTGACCTGGTAAGCTGCCACACACCTGGCAGTGGGGAGCAT
CGCTGTTTCCAATGGCTCAGGAGACAATGAAAAGCCCCCATTTAAAAAAATAACAAACAT
TTTTTAAAAGGCCTCCAATACTCTTATGGAGCCTGGATTTTTCCCACTGCTCTACAGGCT
GTGACTTTTTTTAAGCATCCTGACAGGAAATGTTTTCTTCTACATGGAAAGATAGACAGC
AGCCAACCCTGATCTGGAAGACAGGGCCCCGGCTGGACACACGTGGAACCAAGCCAGGGA
TGGGCTGGCCATTGTGTCCCCGCAGGAGAGATGGGCAGAATGGCCCTAGAGTTCTTTTCC
CTGAGAAAGGAGAAAAAGATGGGATTGCCACTCACCCACCCACACTGGTAAGGGAGGAGA
ATTTGTGCTTCTGGAGCTTCTCAAGGGATTGTGTTTTGCAGGTACAGAAAACTGCCTGTT
ATCTTCAAGCCAGGTTTTCGAGGGCACATGGGTCACCAGTTGCTTTTTCAGTCAATTTGG
CCGGGATGGACTAATGAGGCTCTAACACTGCTCAGGAGACCCCTGCCCTCTAGTTGGTTC
TGGGCTTTGATCTCTTCCAACCTGCCCAGTCACAGAAGGAGGAATGACTCAAATGCCCAA
AACCAAGAACACATTGCAGAAGTAAGACAAACATGTATATTTTTAAATGTTCTAACATAA
GACCTGTTCTCTCTAGCCATTGATTTACCAGGCTTTCTGAAAGATCTAGTGGTTCACACA
GAGAGAGAGAGAGTACTGAAAAAGCAACTCCTCTTCTTAGTCTTAATAATTTACTAAAAT
GGTCAACTTTTCATTATCTTTATTATAATAAACCTGATGCTTTTTTTTAGAACTCCTTAC
TCTGATGTCTGTATATGTTGCACTGAAAAGGTTAATATTTAATGTTTTAATTTATTTTGT
GTGGTAAGTTAATTTTGATTTCTGTAATGTGTTAATGTGATTAGCAGTTATTTTCCTTAA
TATCTGAATTATACTTAAAGAGTAGTGAGCAATATAAGACGCAATTGTGTTTTTCAGTAA
TGTGCATTGTTATTGAGTTGTACTGTACCTTATTTGGAAGGATGAAGGAATGAACCTTTT
TTTCCTAAAAAAAAAAAAAAAAAAAAAAAAAAAAAAAAA
>gi|4507461|gb|NP_003227.1|TGFA 160 aa linear transforming
growth factor, alpha [Homo sapiens].
MVPSAGQLALFALGIVLAACQALENSTSPLSADPPVAAAVVSHFNDCPDSHTQFCFHGTC
RFLVQEDKPACVCHSGYVGARCEHADLLAVVAAAQKKQAITALVVVSIVALAVLIITCVL
IHCCQVRKHCEWCRALICRHEKPSALLKGRTACCHSETVV
>gi|6912653|gb|NM_012433.1|SF3B1 4259 bp mRNA Homo sapiens
splicing factor 3b, subunit 1, 155kDa (SF3B1), mRNA.
ATGGCGAAGATCGCCAAGACTCACGAAGATATTGAAGCACAGATTCGAGAAATTCAAGGC
AAGAAGGCAGCTCTTGATGAAGCTCAAGGAGTGGGCCTCGATTCTACAGGTTATTATGAC
CAGGATTTATGGTGGAGTGACAGCAGATTTGCTGGATACGTGACATCAATTGCTGCTGCA
ACTGAACTTGAAGATGATGACGATGACTATTCATCATCTACGAGTTTGCTTGGTCAGAAG
AAGCCAGGATATCATGCCCCTGTGGCATTGCTTAATGATATACCACAGTCAACAGAACAG
TATGATCCATTTGCTGAGCACAGACCTCCAAAGATTGCAGACCGGGAAGATGAATACAAA
AAGCATAGGCGGACCATGATAATTTCCCCAGAGCGTCTTGATCCTTTTGCAGATGGAGGG
AAGACCCCTGATCCTAAAATGAATGTTAGGACTTACATGGATGTAATGCGAGAACAACAC
TTGACTAAAGAAGAACGAGAAATTAGGCAACAGCTAGCAGAAAAAGCTAAAGCTGGAGAA
CTAAAAGTCGTCAATGGAGCAGCAGCGTCCCAGCCTCCATCAAAACGAAAACGGCGTTGG
GATCAAACAGCTGATCAGACTCCTGGTGCCACTCCCAAAAAACTATCAAGTTGGGATCAG
GCAGAGACCCCTGGGCATACTCCTTCCTTAAGATGGGATGAGACACCAGGTCGTGCAAAG
GGAAGCGAGACTCCTGGAGCAACCCCAGGCTCAAAAATATGGGATCCTACACCTAGCCAC
ACACCAGCGGGAGCTGCTACTCCTGGACGAGGTGATACACCAGGCCATGCGACACCAGGC
CATGGAGGCGCAACTTCCAGTGCTCGTAAAAACAGATGGGATGAAACCCCCAAAACAGAG
AGAGATACTCCTGGGCATGGAAGTGGATGGGCTGAGACTCCTCGAACAGATCGAGGTGGA
GATTCTATTGGTGAAACACCGACTCCTGGAGCCAGTAAAAGAAAATCACGGTGGGATGAA
ACACCAGCTAGTCAGATGGGTGGAAGCACTCCAGTTCTGACCCCTGGAAAGACACCAATT
GGCACACCAGCCATGAACATGGCTACCCCTACTCCAGGTCACATAATGAGTATGACTCCT
GAACAGCTTCAGGCTTGGCGGTGGGAAAGAGAAATTGATGAGAGAAATCGCCCACTTTCT
GATGAGGAATTAGATGCTATGTTCCCAGAAGGATATAAGGTACTTCCTCCTCCAGCTGGT
TATGTTCCTATTCGAACTCCAGCTCGAAAGCTGACAGCTACTCCAACACCTTTGGGTGGT
ATGACTGGTTTCCACATGCAAACTGAAGATCGAACTATGAAAAGTGTTAATGACCAGCCA
TCTGGAAATCTTCCATTTTTAAAACCTGATGATATTCAATACTTTGATAAACTATTGGTT
GATGTTGATGAATCAACACTTAGTCCAGAAGAGCAAAAAGAGAGAAAAATAATGAAGTTG
CTTTTAAAAATTAAGAATGGAACACCACCAATGAGAAAGGCTGCATTGCGTCAGATTACT
GATAAAGCTCGTGAATTTGGAGCTGGTCCTTTGTTTAATCAGATTCTTCCTCTGCTGATG
TCTCCTACACTTGAGGATCAAGAGCGTCATTTACTTGTGAAAGTTATTGATAGGATACTG
TACAAACTTGATGACTTAGTTCGTCCATATGTGCATAAGATCCTCGTGGTCATTGAACCG
CTATTGATTGATGAAGATTACTATGCTAGAGTGGAAGGCCTAGAGATCATTTCTAATTTG
GCAAAGGCTGCTGGTCTGGCTACTATGATCTCTACCATGAGACCTGATATAGATAACATG
GATGAGTATGTCCGTAACACAACAGCTAGAGCTTTTGCTGTTGTAGCCTCTGCCCTGGGC
ATTCCTTCTTTATTGCCCTTCTTAAAAGCTGTGTGCAAAAGCAAGAAGTCCTGGCAAGCG
AGACACACTGGTATTAAGATTGTACAACAGATAGCTATTCTTATGGGCTGTGCCATCTTG
CCACATCTTAGAAGTTTAGTTGAAATCATTGAACATGGTCTTGTGGATGAGCAGCAGAAA
GTTCGGACCATCAGTGCTTTGGCCATTGCTGCCTTGGCTGAAGCAGCAACTCCTTATGGT
ATCGAATCTTTTGATTCTGTGTTAAAGCCTTTATGGAAGGGTATCCGCCAACACAGAGGA
AAGGGTTTGGCTGCTTTCTTGAAGGCTATTGGGTATCTTATTCCTCTTATGGATGCAGAA
TATGCCAACTACTATACTAGAGAAGTGATGTTAATCCTTATTCGAGAATTCCAGTCTCCT
GATGAGGAAATGAAAAAAATTGTGCTGAAGGTGGTAAAACAGTGTTGTGGGACAGATGGT
GTAGAAGCAAACTACATTAAAACAGAGATTCTTCCTCCCTTTTTTAAACACTTCTGGCAG
CACAGGATGGCTTTGGATAGAAGAAATTACCGACAGTTAGTTGATACTACTGTGGAGTTG
GCAAACAAAGTAGGTGCAGCAGAAATTATATCCAGGATTGTGGATGATCTGAAAGATGAA
GCCGAACAGTACAGAAAAATGGTGATGGAGACAATTGAGAAAATTATGGGCAATTTGGGA
GCAGCAGATATTGATCATAAACTTGAAGAACAACTGATTGATGGTATTCTTTATGCTTTC
CAAGAACAGACTACAGAGGACTCAGTAATGTTGAACGGCTTTGGCACAGTGGTTAATGCT
CTTGGCAAACGAGTCAAACCATACTTGCCTCAGATCTGTGGTACAGTTTTGTGGCGTTTA
AATAACAAATCTGCTAAAGTTAGGCAACAGGCAGCTGACTTGATTTCTCGAACTGCTGTT
GTCATGAAGACTTGTCAAGAGGAAAAATTGATGGGACACTTGGGTGTTGTATTGTATGAG
TATTTGGGTGAAGAGTACCCTGAAGTATTGGGCAGCATTCTTGGAGCACTGAAGGCCATT
GTAAATGTCATAGGTATGCATAAGATGACTCCACCAATTAAAGATCTGCTGCCTAGACTC
ACCCCCATCTTAAAGAACAGACATGAAAAAGTACAAGAGAATTGTATTGATCTTGTTGGT
CGTATTGCTGACAGGGGAGCTGAATATGTATCTGCAAGAGAGTGGATGAGGATTTGCTTT
GAGCTTTTAGAGCTCTTAAAAGCCCACAAAAAGGCTATTCGTAGAGCCACAGTCAACACA
TTTGGTTATATTGCAAAGGCCATTGGCCCTCATGATGTATTGGCTACACTTCTGAACAAC
CTCAAAGTTCAAGAAAGGCAGAACAGAGTTTGTACCACTGTAGCAATAGCTATTGTTGCA
GAAACATGTTCACCCTTTACAGTACTCCCTGCCTTAATGAATGAATACAGAGTTCCTGAA
CTGAATGTTCAAAATGGAGTGTTAAAATCGCTTTCCTTCTTGTTTGAATATATTGGTGAA
ATGGGAAAAGACTACATTTATGCCGTAACACCGTTACTTGAAGATGCTTTAATGGATAGA
GACCTTGTACACAGACAGACGGCTAGTGCAGTGGTACAGCACATGTCACTTGGGGTTTAT
GGATTTGGTTGTGAAGATTCGCTGAATCACTTGTTGAACTATGTATGGCCCAATGTATTT
GAGACATCTCCTCATGTAATTCAGGCAGTTATGGGAGCCCTAGAGGGCCTGAGAGTTGCT
ATTGGACCATGTAGAATGTTGCAATATTGTTTACAGGGTCTGTTTCACCCAGCCCGGAAA
GTCAGAGATGTATATTGGAAAATTTACAACTCCATCTACATTGGTTCCCAGGACGCTCTC
ATAGCACATTACCCAAGAATCTACAACGATGATAAGAACACCTATATTCGTTATGAACTT
GACTATATCTTATAATTTTATTGTTTATTTTGTGTTTAATGCACAGCTACTTCACACCTT
AAACTTGCTTTGATTTGGTGATGTAAACTTTTAAACATTGCAGTTCAGTGTAGAACTGGT
CATAGAGGAAGAGCTAGAAATCCAGTAGCATGATTTTTAAATAACCTGTCTTTGTTTTTG
ATGTTAAACAGTAAATGCCAGTAGTGACCAAGAACACAGTGATTATATACACTATACTGG
AGGGATTTCATTTTTAATTCATCTTTATGAAGATTTAGAACTCATTCCTTGTGTTTAAAG
GGAATGTTTAATTGAGAAATAAACATTTGTGTACAAAATGCTAAAAAAAAAAAAAAAAA
>gi|6912654|gb|NP_036565.1|SF3B1 1304 aa linear splicing
factor 3b, subunit 1, 155kDa; spliceosome-associated factor
155; splicing factor 3b, subunit 1, 155kD [Homo sapiens].
MAKTAKTHEDIEAQIREIQGKKAALDEAQGVGLDSTGYYDQEIYGGSDSRFAGYVTSIAA
TELEDDDDDYSSSTSLLGQKKPGYHAPVALLNDIPQSTEQYDPFAEHRPPKIADREDEYK
KHRRTMIISPERLDPFADGGKTPDPKMNVRTYMDVMREQHLTKEEREIRQQLAEKAKAGE
LKVVNGAAASQPPSKRKRRWDQTADQTPGATPKKLSSWDQAETPGHTPSLRWDETPGRAK
GSETPGATPGSKIWDPTPSHTPAGAATPGRGDTPGHATPGHGGATSSARKNRWDETPKTE
RDTPGHGSGWAETPRTDRGGDSIGETPTPGASKRKSRWDETPASQMGGSTPVLTPGKTPI
GTPAMNMATPTPGHIMSMTPEQLQAWRWEREIDERNRPLSDEELDAMFPEGYKVLPPPAG
YVPIRTPARKLTATPTPLGGMTGFHMQTEDRTMKSVNDQPSGNLPFLKPDDIQYFDKLLV
DVDESTLSPEEQKERKIMKLLLKIKNGTPPMRKAALRQITDKAREFGAGPLFNQILPLLM
SPTLEDQERRLLVKVIDRILYKLDDLVRPYVHKILVVIEPLLIDEDYYARVEGLEIISNL
AKAAGLATMISTMRPDIDNMDEYVRNTTARAFAVVASALGIPSLLPFLKAVCKSKKSWQA
RHTGIKIVQQIAILMGCAILPHLRSLVEIIEHGLVDEQQKVRTISALAIAALAEAATPYG
IESFDSVLKPLWKGIRQHRGKGLAAFLKAIGYLIPLMDAEYANYYTREVMLILIREFQSP
DEEMKKIVLKVVKQCCGTDGVEANYIKTEILPPFFKHFWQHRMALDRRNYRQLVDTTVEL
ANKVGAAEIISRIVDDLKDEAEQYRKMVMETIEKIMGNLGAADIDHKLEEQLIDGILYAF
QEQTTEDSVMLNGFGTVVNALGKRVKPYLPQICGTVLWRLNNKSAKVRQQAADLISRTAV
VMKTCQEEKLMGHLGVVLYEYLGEEYPEVLGSILGALKAIVNVIGMHKMTPPIKDLLPRL
TPILKNRHEKVQENCIDLVGRIADRGAEYVSAREWMRICFELLELLKAHKKAIRRATVNT
FGYIAKAIGPHDVLATLLNNLKVQERQNRVCTTVAIAIVAETCSPFTVLPALMNEYRVPE
LNVQNGVLKSLSFLFEYIGEMGKDYIYAVTPLLEDALMDRDLVHRQTASAVVQHMSLGVY
GFGCEDSLNRLLNYVWPNVFETSPHVIQAVMGALEGLRVAIGPCRMLQYCLQGLFHPARK
VRDVYWKIYNSIYIGSQDALIAHYPRIYNDDKNTYIRYELDYIL
>gi|21707321|gb|BC033864.1|BC033864 2321 bp mRNA Homo
sapiens, Similar to branched chain aminotransferase 1,
cytosolic, clone MGC:45234 IMAGE:5186262, mRNA, complete
cds.
GGTGGATGCTGCGGCATCGGAGGACCCTGCTGGTGGAGGAAATGGTTCACGCCCGTCCCC
GTTCCCTTTGCAGGCTTGCTATTGTGCGTCTGTGATTGACAAGACCACGAGGCTGAGCGC
GCCCTGGAGATTTTTCTATAAATGGCTTAACACCCCAGTCTAGACTATTTGCTCGGATAT
AAGGGAGACAATTGTTTTTTTGTTCTTTGCCGGCGAACCCTGGCTCTGTAGGGCTGACCT
GGAATTTAACCAGTCTTCCCTGAGCCGGCGGAGGAGGACAAAAACCGCCGCGACCCCGGC
AGGGTGGGAAGTGCAGGGCAGCGCTCCCAAGACACGCTTGTTGGAGGTTCGGGCCTGGGT
GCTTGGTTGTCTGAGCCTCCTTTTTTGTGTTTGCCTGGGTCCTGGAGAGGAGCGCACGGT
ATCATGGATTGCAGTAACGGATGCTCCGCAGAGTGTACCGGAGAAGGAGGATCAAAAGAG
GTGGTGGGGACTTTTAAGGCTAAAGACCTAATAGTCACACCAGCTACCATTTTAAAGGAA
AAACCAGACCCCAATAATCTGGTTTTTGGAACTGTGTTCACGGATCATATGCTGACGGTG
GAGTGGTCCTCAGAGTTTGGATGGGAGAAACCTCATATCAAGCCTCTTCAGAACCTGTCA
TTGCACCCTGGCTCATCAGCTTTGCACTATGCAGTGGAATTATTTGAAGGATTGAAGGCA
TTTCGAGGAGTAGATAATAAAATTCGACTGTTTCAGCCAAACCTCAACATGGATAGAATG
TATCGCTCTGCTGTGAGGGCAACTCTGCCGGTATTTGACAAAGAAGAGCTCTTAGAGTGT
ATTCAACAGCTTGTGAAATTGGATCAAGAATGGGTCCCATATTCAACATCTGCTAGTCTG
TATATTCGTCCTACATTCATTGGAACTGAGCCTTCTCTTGGAGTCAAGAAGCCTACCAAA
GCCCTGCTCTTTGTACTCTTGAGCCCAGTGGGACCTTATTTTTCAAGTGGAACCTTTAAT
CCAGTGTCCCTGTGGGCCAATCCCAAGTATGTAAGAGCCTGGAAAGGTGGAACTGGGGAC
TGCAAGATGGGAGGGAATTACGGCTCATCTCTTTTTGCCCAATGTGAAGCAGTAGATAAT
GGGTGTCAGCAGGTCCTGTGGCTCTATGGAGAGGACCATCAGATCACTGAAGTGGGAACT
ATGAATCTTTTTCTTTACTGGATAAATGAAGATGGAGAAGAAGAACTGGCAACTCCTCCA
CTAGATGGCATCATTCTTCCAGGAGTGACAAGGCGGTGCATTCTGGACCTGGCACATCAG
TGGGACACAGAACTCAGCTTGTTTTCAATTAATTTGCCTGATTTTCTGCAGTTCATTTAC
TTTTGAACAACATAATTGCAATTGTAGACTGAGAGAAATTGAAACTTTCAAAGAGCCATA
TTTCTATTGCAGATATATTTTCCTGCTCTTCCAAATCTACTTACAGCATGAGTTCTTCTT
TTAAATATTCAAATATTTTGAATATTGCCAAGAGCTTTGATTTCCATTTTTATCTCTTGT
GGGTTTATAAATTAAGAAAAAATACTCATCTTATTTTTTTAAACCTCTCTATTTTTATTG
CCCTTTATTCAAATAACTTGTTGACAAACTTTGAACTTGAACCACTGAGGTAAAAGAACA
AGAATTAAACAGATAGTTTAAACACATAGCTTAAAAGGATCTTTTTCCCATTTCCTATCC
TTGAGCAAAGAATATATTCAAACACTTTGGCAGAAGTCAATGAGGTTATACCACTAATTC
CATGATGAAAATCAACTGAATGTGATACTGAAAGAGAAGGAAGAGAATTGTCACTGTAAA
GTCAACTGTTAGTCATATTAGGAAAAAAAATACATACAATACAATTTCTCAAATAAAGTC
CAAATATACATTCAATGTTTAAAAATAATGAGTATTTCAGATATTTGAACTCAGTCTGTT
CTTTATTCCATAAAAGATATAGGTAAGCCGTGCACGGTGGCTCACAACTATAATCCCAGC
ACTTTGGCACTTTGGGAGGCTGAGGTGGGAGGATCACATGAGCCCAGCCTGGGCAACATA
GGGAGACCGCTATCTTTACAAAATAAAATATAAAATATAAAACCTAGTTGGGCATGGCAG
CATACACCTGTAGTCCCAGGTGCTCGGGAGACTGAGACAGGAGGATCGCTTGGGCCTGGG
AGGTCGAGGCTGCAGTGAGCCAAGATTATGCCACTGCATTCCAGCCTGGGTGACAGGGCA
AGACCCTGTCTTAAAAAAAAAAAAAAAAAAAAAAAAAAAAA
>gi|21707322|gb|AAH33864.1|AAH33864 320 aa linear Similar to
branched chain aminotransferase 1, cytosolic [Homo sapiens].
MDCSNGCSAECTGEGGSKEVVGTFKAKDLIVTPATILKEKPDPNNLVFGTVFTDHMLTVE
WSSEFGWEKPHIKPLQNLSLHPGSSALHYAVELFEGLKAFRGVDNKIRLFQPNLNMDRMY
RSAVRATLPVFDKEELLECIQQLVKLDQEWVPYSTSASLYIRPTFIGTEPSLGVKKPTKA
LLFVLLSPVGPYFSSGTFNPVSLWANPKYVRAWKGGTGDCKMGGNYGSSLFAQCEAVDNG
CQQVLWLYGEDHQITEVGTMNLFLYWINEDGEEELATPPLDGIILPGVTRRCILDLAHQW
DTELSLFSINLPDFLQFIYF
>gi|29570794|gb|NM_001895.2|CSNK2A1 2323 bp mRNA Homo
sapiens casein kinase 2, alpha 1 polypeptide (CSNK2A1),
transcript variant 2, mRNA.
CCCGCCTCCTGGTAGGAGGGGGTTTCCGCTTCCGGCAGCAGCGGCTGCAGCCTCGCTCTG
GTCCCTGCGGCTGGCGGCCGAGCCGTGTGTCTCCTCCTCCATCGCCGCCATATTGTCTGT
GTGAGCAGAGGGGAGAGCGGCCGCCGCCGCTGCCGCTTCCACCACAGTTTGAAGAAAACA
GGTCTGAAACAAGGTCTTACCCCCAGCTGCTTCTGAACACAGTGACTGCCAGATCTCCAA
ACATCAAGTCCAGCTTTGTCCGCCAACCTGTCTGACATGTCGGGACCCGTGCCAAGCAGG
GCCAGAGTTTACACAGATGTTAATACACACAGACCTCGAGAATACTGGGATTACGAGTCA
CATGTGGTGGAATGGGGAAATCAAGATGACTACCAGCTGGTTCGAAAATTAGGCCGAGGT
AAATACAGTGAAGTATTTGAAGCCATCAACATCACAAATAATGAAAAAGTTGTTGTTAAA
ATTCTCAAGCCAGTAAAAAAGAAGAAAATTAAGCGTGAAATAAAGATTTTGGAGAATTTG
AGAGGAGGTCCCAACATCATCACACTGGCAGACATTGTAAAAGACCCTGTGTCACGAACC
CCCGCCTTGGTTTTTGAACACGTAAACAACACAGACTTCAAGCAATTGTACCAGACGTTA
ACAGACTATGATATTCGATTTTACATGTATGAGATTCTGAAGGCCCTGGATTATTGTCAC
AGCATGGGAATTATGCACAGAGATGTCAAGCCCCATAATGTCATGATTGATCATGAGCAC
AGAAAGCTACGACTAATAGACTGGGGTTTGGCTGAGTTTTATCATCCTGGCCAAGAATAT
AATGTCCGAGTTGCTTCCCGATACTTCAAAGGTCCTGAGCTACTTGTAGACTATCAGATG
TACGATTATAGTTTGGATATGTGGAGTTTGGGTTGTATGCTGGCAAGTATGATCTTTCGG
AAGGAGCCATTTTTCCATGGACATGACAATTATGATCAGTTGGTGAGGATAGCCAAGGTT
CTGGGGACAGAAGATTTATATGACTATATTGACAAATACAACATTGAATTAGATCCACGT
TTCAATGATATCTTGGGCAGACACTCTCGAAAGCGATGGGAACGCTTTGTCCACAGTGAA
AATCAGCACCTTGTCAGCCCTGAGGCCTTGGATTTCCTGGACAAACTGCTGCGATATGAC
CACCAGTCACGGCTTACTGCAAGAGAGGCAATGGAGCACCCCTATTTCTACACTGTTGTG
AAGGACCAGGCTCGAATGGGTTCATCTAGCATGCCAGGGGGCAGTACGCCCGTCAGCAGC
GCCAATATGATGTCAGGGATTTCTTCAGTGCCAACCCCTTCACCCCTTGGACCTCTGGCA
GGCTCACCAGTGATTGCTGCTGCCAACCCCCTTGGGATGCCTGTTCCAGCTGCCGCTGGC
GCTCAGCAGTAACGGCCCTATCTGTCTCCTGATGCCTGAGCAGAGGTGGGGGAGTCCACC
CTCTCCTTGATGCAGCTTGCGCCTGGCGGGGAGGGGTGAAACACTTCAGAAGCACCGTGT
CTGAACCGTTGCTTGTGGATTTATAGTAGTTCAGTCATAAAAAAAAAATTATAATAGGCT
GATTTTCTTTTTTCTTTTTTTTTTTAACTCGAACTTTTCATAACTCAGGGGATTCCCTGA
AAAATTACCTGCAGGTGGAATATTTCATGGACAAATTTTTTTTTCTCCCCTCCCAAATTT
AGTTCCTCATCACAAAAGAACAAAGATAAACCAGCCTCAATCCCGGCTGCTGCATTTAGG
TGGAGACTTCTTCCCATTCCCACCATTGTTCCTCCACCGTCCCACACTTTAGGGGGTTGG
TATCTCGTGCTCTTCTCCAGAGATTACAAAAATGTAGCTTCTCAGGGGAGGCAGGAAGAA
AGGAAGGAAGGAAAGAAGGAAGGGAGGACCCAATCTATAGGAGCAGTGGACTGCTTGCTG
GTCGCTTACATCACTTTACTCCATAAGCGCTTCAGTGGGGTTATCCTAGTGGCTCTTGTG
GAAGTGTGTCTTAGTTACATCAAGATGTTGAAAATCTACCCAAAATGCAGACAGATACTA
AAAACTTCTGTTCAGTAAGAATCATGTCTTACTGATCTAACCCTAAATCCAACTCATTTA
TACTTTTATTTTTAGTTCAGTTTAAAATGTTGATACCTTCCCTCCCAGGCTCCTTACCTT
GGTCTTTTCCCTGTTCATCTCCCAACATGCTGTGCTCCATAGCTGGTAGGAGAGGGAAGG
CAAAATCTTTCTTAGTTTTCTTTGTCTTGGCCATTTTGAATTC
>gi|4503095|gb|NP_001886.1|CSNK2A1 391 aa linear casein
kinase II alpha 1 subunit isoform a; CK2 catalytic subunit
alpha [Homo sapiens].
MSGPVPSRARVYTDVNTHRPREYWDYESHVVEWGNQDDYQLVRKLGRGKYSEVFEAINIT
NNEKVVVKILKPVKKKKIKREIKILENLRGGPNIITLADIVKDPVSRTPALVFEHVNNTD
FKQLYQTLTDYDIRFYMYEILKALDYCHSMGIMHRDVKPHNVMIDHEHRKLRLIDWGLAE
FYHPGQEYNVRVASRYFKGPELLVDYQMYDYSLDMWSLGCMLASMIFRKEPFFHGHDNYD
QLVRIAKVLGTEDLYDYIDKYNIELDPRFNDILGRHSRKRWERFVHSENQHLVSPEALDF
LDKLLRYDHQSRLTAREAMEHPYFYTVVKDQARMGSSSMPGGSTPVSSANMMSGISSVPT
PSPLGPLAGSPVIAAANPLGMPVPAAAGAQQ
>gi|13375963|gb|NM_024689.1|FLJ14103 2502 bp mRNA Homo
sapiens hypothetical protein FLJ14103 (FLJ14103), mRNA.
CTCTTTGGCCAAGCCCTGCCTCTGTACAGCCTCGAGTGGACAGCCAGAGGCTGCAGCTGG
AGCCCAGAGCCCAAGATGGAGCCCCAGCTGGGGCCTGAGGCTGCCGCCCTCCGCCCTGGC
TGGCTGGCCCTGCTGCTGTGGGTCTCAGCCCTGAGCTGTTCTTTCTCCTTGCCAGCTTCT
TCCCTTTCTTCTCTGGTGCCCCAAGTCAGAACCAGCTACAATTTTGGAAGGACTTTCCTC
GGTCTTGATAAATGCAATGCCTGCATCGGGACATCTATTTGCAAGAAGTTCTTTAAAGAA
GAAATAAGATCTGACAACTGGCTGGCTTCCCACCTTGGACTGCCTCCCGATTCCTTGCTT
TCTTATCCTGCAAATTACTCAGATGATTCCAAAATCTGGCGCCCTGTGGAGATCTTTAGA
CTGGTCAGCAAATATCAAAACGAGATCTCAGACAGGAAAATCTGTGCCTCTGCATCAGCC
CCAAAGACCTGCAGCATTGAGCGTGTCCTGCGGAAAACAGAGAGGTTCCAGAAATGGCTG
CAGGCCAAGCGCCTCACGCCGGACCTGGTGCAGGACTGTCACCAGGGCCAGAGAGAACTA
AAGTTCCTGTGTATGCTGAGATAACACCAGTGAAAAAGCCTGGCATGGAGCCCAGCACTG
AGAACTTCCAGAAAGTGTTAGCCTTCTCCCAACTGTGTTATACCAACCACATTTTCAAAT
AGTAATCATTAAAGAGGCTTCTGCATCAAACCTTCACATGCAGCTCCCATGCCACCCTCC
AGAATTCACCAACACACAGGCCCACCAGCAACAGGCTACCTTTGCACAATATTCTCTGAT
GACAACTCCAAAGCCCCGGCTCTTTCCACCACACTGTGGTCCCCTAGATGGGGCTGTTGC
TGAGCCCACCCCAATCCAGATGTGATCCCCCTGTGATCTACTTCTGGCAAGATTCTCAGT
CTGGACAGGTCTTCCCTATGAGATAGAACCTGATAAGGAGCTAGGGCAATTCTGACAACA
TTACCAAAGGCCCACATAACTTCTAAATTTTGGTCTGGTCTGAAGGAAAACCTGTTCTCG
CCCTAGTGATGGATGAACTCTCTTATCTCTGGCTTCTAGAGGGAAAAAAAAAGCATACCT
CTTTTACTTTTTAAGTACCTCCATCAGAGTCATGAAATCACCTGTCAAGACTATCTATCT
TTTATGTTTCCATTCTGGTAAGAACTCTTTAAATGAGGACACTGCTGATTGCTGGTGATG
TTTTTTGAGCAAACACTCGGGGGTATGGATGAAAGCCAATCGCAGGTCAAATGACTCCTT
GGGGAAGCTACTTCTCCTCTATTCAGATTTCACTAAAATCTTCCAAGATGAAAGCAAATC
TAGATTTCGGTCTTCATTGCTGTCCATTTTTGTAATGAACGAGTGTTTTTCCTTTAGCTA
GTGTATCAGGCAGGGTTCTACCAGAGAAACAGAACCAGTAGGAGATACATATACATGTCC
AGATTTATTTCAAAGAATTGATTTACATGATTGTGGGGATTGGCAAGTCCAAAATCCATA
TGGTAGGCCTGCAATCTGTAAACCTTTGGGCAGGAGCTGATGCTGTAGTTTGCAGATAGA
ATTCCTTGTTCCTTAAAAAAATCTGTTTTTGTTCTTAAGGGCTTTGAATGATTGGATCAG
GCCCACCCAGATTACCTAGATAATCTCTTTTACTTAAAGTAAACTGATTGTAGGTGCTAA
TCACATCTATGAAATGCCTTCACAGCAACACCTAGATTAGCATTCAATTGAATAACTGGG
GAATACAGCCTAGCCAAGTTGACACATAAAATTAACCATCACAGCAACATGCCTGCTAAA
TTTTATCGACCGTCTTCAGACTGTTAAGGATTGTGGTAGAGAACTGTGACAGCCACTCTC
AGCATCACCCTGAACCAAAGGCCCCTATCAAGTAACAATATAGCCAAGCAAAATTCCAGT
CAATAGAGACATTGACTGGTTGGCTGGCTTCCCAAGGGATAGCACCAGACAAGAAATGCA
AGGATGAGGAAACCAGGCACGGGAGAGGGAGGGGCAACAGAGGTCCAGGGTTTGGTTATC
TTTTTATTTTTCACTGGGAGGTGGTAAGTTAGCCCTGTTGCCCATGTATGCAGATGGGAG
AAGTGATTTAGAAACTCCAAAGCAATTGGTAATCCCCAAAATGGGTGTATCTGGTTTGAA
ATGAAACCTTATTTTATTGGAAATGGTTGGTTTCCCAATTCTGTTTGCCATTGGCCAATA
TAATTGTGGGTTTGCACATGGCCAGCACATGCCAAACAGAAGTAGACAAAGGTCTCACTC
TGTAAGTGGGACCTTGGGGAGGAGCTGCCTCCATCATAAAGGGAGGGGTTAGTAAAAATG
GTCTCTTAAGCCTGTTCCTGCTACAGTTATAGAGGTTGCTCAGAACCTTCTCAGCAAATA
TAGCAGTTATCTATTGTTGTGTATTAAACCATTTCAACACAT
>gi|13375964|gb|NP_078965.1|FLJ14103 182 aa linear hypo-
thetical protein FLJ14103 [Homo sapiens].
MEPQLGPEAAALRPGWLALLLWVSALSCSFSLPASSLSSLVPQVRTSYNFGRTFLGLDKC
NACIGTSICKKFFKEEIRSDNWLASHLGLPPDSLLSYPANYSDDSKIWRPVEIFRLVSKY
QNEISDRKICASASAPKTCSIERVLRKTERFQKWLQAKRLTPDLVQDCHQGQRELKFLCM
LR
>gi|7658290|gb|AF221842.1|AF221842 3057 bp mRNA Homo sapiens
U5 snRNP-associated 102 kDa protein mRNA., complete cds.
ACTTTGCTACGGAGTGCATCGGACGTCGAAGCCTAGAGTCTCTGCGTCTTTCCCTCTTCC
GCTGCCTCATTCCTTTCCTTCCTAGCCTTGGTCGTCGCCGCCACCATGAACAAGAAGAAG
AAACCGTTCCTAGGGATGCCCGCGCCCCTCGGCTACGTGCCGGGGCTGGGCCGGGGCGCC
ACTGGCTTCACCACGCGGTCAGACATTGGGCCCGCCCGTGATGCAAATGACCCTGTGGAT
GATCGCCATGCACCCCCAGGCAAGAGAACCGTTGGGGACCAGATGAAGAAAAATCAGGCT
GCTGACGATGACGACGAGGATCTAAATGACACCAATTACGATGAGTTTAATGGCTATGCT
GGGAGCCTCTTCTCAAGTGGACCCTACGAGAAAGATGATGAGGAAGCAGATGCTATCTAT
GCAGCCCTGGATAAAAGGATGGATGAAAGAAGAAAAGAAAGACGGGAGCAAAGGGAGAAA
GAAGAAATAGAGAAATATCGTATGGAACGCCCCAAAATCCAACAGCAGTTCTCAGACCTC
AAGAGGAAGTTGGCAGAAGTCACAGAAGAAGAGTGGCTGAGCATCCCCGAGGTTGGCGAT
GCCAGAAATAAACGTCAGCGGAACCCACGCTATGAGAAGCTGACCCCTGTTCCTGACAGT
TTCTTTGCCAAACATTTACAGACCGGAGAGAACCATACCTCAGTGGATCCCCGACAAACT
CAATTTGGAGGTCTTAACACACCCTATCCAGGTGGACTAAACACTCCATACCCAGGTGGA
ATGACGCCAGGACTGATGACACCTGGCACAGGTGAGCTGGACATGAGGAAGATTGGCCAA
GCGAGGAACACTCTGATGGACATGAGGCTGAGCCAGGTGTCTGACTCCGTGAGTGGACAG
ACCGTCGTTGACCCCAAAGGCTACCTGACGGATTTAAATTCCATGATCCCGACACACGGA
GGAGACATCAATGATATCAAGAAGGCGCGACTGCTCCTCAAGTCTGTTCGGGAGACGAAC
CCTCATCACCCGCCAGCCTGGATTGCATCAGCCCGCCTGGAAGAAGTCACTGGGAAGCTA
CAAGTAGCTCGGAACCTTATCATGAAGGGGACGGAGATGTGCCCCAAGAGTGAAGATGTC
TGGCTGGAAGCAGCCAGGTTGCAGCCTGGGGACACAGCCAAGGCCGTGGTAGCCCAAGCT
GTCCGTCATCTCCCACAGTCTGTCAGGATTTACATCAGAGCCGCAGAGCTGGAAACGGAC
ATTCGTGCAAAGAAGCGGGTTCTTCGGAAAGCCCTCGAGCATGTTCCAAACTCGGTTCGC
TTGTGGAAAGCAGCCGTTGAGCTGGAAGAACCTGAAGATGCTAGAATCATGCTGAGCCGA
GCTGTGGAGTGCTGCCCCACCAGCGTGGAGCTCTGGCTTGCTCTGGCAAGGCTGGAGACC
TATGAAAATGCCCGCAAGGTCTTGAACAAGGCGCGGGAGAACATTCCTACAGACCGACAT
ATCTGGATCACGGCTGCTAAGCTGGAGGAAGCCAATGGGAACACGCAGATGGTGGAGAAG
ATCATCGACCGAGCCATCACCTCGCTGCGGGCCAACGGTGTGGAGATCAACCGTGAGCAG
TGGATCCAGGATGCCGAGGAATGTGACAGGGCTGGGAGTGTGGCCACCTGCCAGGCCGTC
ATGCGTGCCGTGATTGGGATTGGGATTGAGGAGGAAGATCGGAAGCATACCTGGATGGAG
GATGCTGACAGTTGTGTAGCCCACAATGCCCTGGAGTGTGCACGAGCCATCTACGCCTAC
GCCCTGCAGGTGTTCCCCAGCAAGAAGAGTGTGTGGCTGCGCGCCGCGTACTTCGAGAAG
AACCATGGCACTCGGGAGTCCCTGGAAGCACTCCTGCAGAGGGCTGTGGCCCACTGCCCC
AAAGCAGAGGTGCTGTGGCTCATGGGCGCCAAGTCCAAGTGGCTGGCAGGGGATGTGCCT
GCAGCAAGGAGCATCCTGGCCCTGGCCTTCCAGGCCAACCCCAACAGTGAGGAGATCTGG
CTGGCAGCCGTGAAGCTGGAGTCCGAGAATGATGAGTACGAGCGGGCCCGGAGGCTGCTG
GCCAAGGCGCGGAGCAGTGCCCCCACCGCCCGGGTGTTCATGAAGTCTGTGAAGCTGGAG
TGGGTGCAAGACAACATCAGGGCAGCCCAAGATCTGTGCGAGGAGGCCCTGCGGCACTAT
GAGGACTTCCCCAAGCTGTGGATGATGAAGGGGCAGATCGAGGAGCAGAAGGAGATGATG
GAGAAGGCGCGGGAAGCCTATAACCAGGGGTTGAAGAAGTGTCCCCACTCCACACCCCTG
TGGCTTTTGCTCTCTCGGCTGGAGGAGAAGATTGGGCAGCTTACTCGAGCACGGGCCATT
TTGGAAAAGTCTCGTCTGAAGAACCCAAAGAACCCTGGGCTGTGGTTGGAGTCCGTGCGG
CTGGAGTACCGTGCGGGGCTGAAGAACATCGCAAATACACTCATGGCCAAGGCGCTGCAG
GAGTGCCCCAACTCCGGTATCCTGTGGTCTGAGGCCATCTTCCTCGAGGCAAGGCCCCAG
AGGAGGACCAAGAGCGTGGATGCCCTGAAGAAGTGTGAGCATGACCCCCATGTGCTCCTG
GCCGTGGCCAAGCTGTTTTGGAGTCAGCGGAAGATCACCAAGGCCAGGGAGTGGTTCCAC
CGCACTGTGAAGATTGACTCGGACCTGGGGGATGCCTGGGCCTTCTTCTACAAGTTTGAG
CTGCAGCATGGCACTGAGGAGCAGCAGGAGGAGGTGAGGAAGCGCTGTGAGAGTGCAGAG
CCTCGGCATGGGGAGCTGTGGTGCGCCGTGTCCAAGGACATCGCCAACTGGCAGAAGAAG
ATCGGGGACATCCTTAGGCTGGTGGCCGGCCGCATCAAGAACACCTTCTGATTGAGCGGT
TGCCATGGCCGGTCTCCGTGGGGCAGGGTTGGGCCGCATGTGGAAGGGCTCTGAGCTGTG
TCCTCCTTCATTAAAAGTTTTTATGTCTCGTGTCAGAAAAAAAAAAAAAAAAAAAAA
>gi|7658291|gb|AAF66128.1|AAF66128 941 aa linear U5 snRNP-
associated 102 kDa protein [Homo sapiens].
MNKKKKPFLGMPAPLGYVPGLGRGATGFTTRSDIGPARDANDPVDDRHAPPGKRTVGDQM
KKNQAADDDDEDLNDTNYDEFNGYAGSLFSSGPYEKDDEEADAIYAALDKRNDERRKERR
EQREKEEIEKYRMERPKIQQQFSDLKRKLAEVTEEEWLSIPEVGDARNKRQRNPRYEKLT
PVPDSFFAKHLQTGENHTSVDPRQTQFGGLNTPYPGGLNTPYPGGMTPGLMTPGTGELDM
RKIGQARNTLMDMRLSQVSDSVSGQTVVDPKGYLTDLNSMIPTHGGDINDIKKARLLLKS
VRETNPHHPPAWIASARLEEVTGKLQVARNLIMKGTEMCPKSEDVWLEAARLQPGDTAKA
VVAQAVRHLPQSVRIYIRAAELETDIRAKKRVLRKALEHVPNSVRLWKAAVELEEPEDAR
IMLSRAVECCPTSVELWLALARLETYENARKVLNKARENIPTDRHIWITAAKLEEANGNT
QMVEKIIDRAITSLRANGVEINREQWIQDAEECDRAGSVATCQAVMRAVIGIGIEEEDRK
HTWMEDADSCVAHNALECARAIYAYALQVFPSKKSVWLRAAYFEKNRGTRESLEALLQRA
VAHCPKAEVLWLMGAKSKWLAGDVPAARSILALAFQANPNSEEIWLAAVKLESENDEYER
ARRLLAKARSSAPTARVFMKSVKLEWVQDNIRAAQDLCEEALRHYEDFPKLWMMKGQIEE
QKEMMEKAREAYNQGLKKCPHSTPLWLLLSRLEEKIGQLTRARAILEKSRLKNPKNPGLW
LESVRLEYRAGLKNIANTLMAKALQECPNSGILWSEAIFLEARPQRRTKSVDALKKCEHD
PHVLLAVAKLFWSQRKITKAREWFHRTVKIDSDLGDAWAFFYKFELQHGTEEQQEEVRKR
CESAEPRHGELWCAVSKDIANWQKKIGDILRLVAGRIKNTF
>gi|5454165|gb|NM_006370.1|VTI1B 1287 bp mRNA Homo sapiens
vesicle transport through interaction with t-SNAREs homolog
1B (yeast) (VTI1B), mRNA.
CCCTTTCGCTGCGGCCTTTCCCCAACCCGGACCCGGCACTTCTCGGGTTCCGCGACTGCC
GATCGCCCCGGCGCGGCACCGCTCCCTCAGGAGTCGCCTAGGCCGCGCAGTCTCCCGACT
TCTCGTCAGGCTTTCGCGCCGGCGCTCCAGCAATCACTGGCTGGAGAAGGTGGGCGTTCC
GGCTCGAGAGGACCCTGCCGCGGCTCCGGAAGAGCCTCGTCCTGGGCGGCGGTGGTGCGG
CGGTCGCCGTTATGGCCACTGGGCTGGGCGGCTGACCGCGGGCTAGGAAAGGGCCCAGGG
CCCGAATCTCGGTGGCCGCTGCTCCAGCGCGGCCTGCGCCATGGCCTCCTCCGCCGCCTC
CTCGGAGCATTTCGAGAAGCTGCACGAGATCTTCCGCGGCCTCCATGAAGACCTACAAGG
GGTGCCCGAGCGGCTGCTGGGGACGGCGGGGACCGAAGAAAAGAAGAAATTGATCAGGGA
TTTTGATGAAAAGCAACAGGAAGCAAATGAAACGCTGGCAGAGATGGAGGAGGAGCTACG
TTATGCACCCCTGTCTTTCCGAAACCCCATGATGTCTAAGCTTCGAAACTACCGGAAGGA
CCTTGCTAAACTCCATCGGGAGGTGAGAAGCACACCTTTGACAGCCACACCTGGAGGCCG
AGGAGACATGAAATATGGCATATATGCTGTAGAGAATGAGCATATGAATCGGCTACAGTC
TCAAAGGGCAATGCTTCTGCAGGGCACTGAAAGCCTGAACCGGGCCACCCAAAGTATTGA
ACGTTCTCATCGGATTGCCACAGAGACTGACCAGATTGGCTCAGAAATCATAGAAGAGCT
GGGGGAACAACGAGACCAGTTAGAACGTACCAAGAGTAGACTGGTAAACACAAGTGAAAA
CTTGAGCAAAAGTCGGAAGATTCTCCGTTCAATGTCCAGAAAAGTGACAACCAACAAGCT
GCTGCTTTCCATTATCATCTTACTGGAGCTCGCCATCCTGGGAGGCCTGGTTTACTACAA
ATTCTTTCGCAGCCATTGAACTTCTATAGGGAAGGGTTTGTGGACCAGAACTTTGACCTT
GTGAATGCATGATGTTAGGGATGTGGATAGAATAAGCATATTGCTGCTGTGGGCTGACAG
TTCAAGGATGCACTGTATAGCCAGGCTGTGGGAGGAGGGAGGAAAGATGAAAAACCACTT
AAATGTGAAGGAACAACAGCAACAAGACCAGTATGATATACCAAGGTAATAAATGCTGTT
TATGACTTCTTTAAAAAAAAAAAAAAA
>gi|5454166|gb|NP_006361.1|VTI1B 232 aa linear vesicie-
associated soluble NSF attachment protein receptor (v-SN;
vesicle-associated soluble NSF attachment protein receptor
(v-SNARE; homolog of S. cerevisiae VTI1) [Homo sapiens].
MASSAASSEHFEKLHEIFRGLHEDLQGVPERLLGTAGTEEKKKLIRDFDEKQQEANETLA
EMEEELRYAPLSFRNPMMSKLRNYRKDLAKLHREVRSTPLTATPGGRGDMKYGIYAVENE
HMNRLQSQRAMLLQGTESLNRATQSIERSHRIATETDQIGSEIIEELGEQRDQLERTKSR
LVNTSENLSKSRKILRSMSRKVTTNKLLLSIIILLELAILGGLVYYKFFRSH
>gi|7705992|gb|NM_016440.1|LOC51231 1869 bp mRNA Homo
sapiens VRK3 for vaccinia related kinase 3 (LOC51231), mRNA.
CCGAGGGTCAGGCTGCAGAAGCCCAGAATCCCACCCCAGTCCCCAAGTACAGAGGTCGCT
GTCAAGATGGAGTTTCCAACCCAGTAAATCCAAGGGCCAGACCGTGACCTCATAAAGCAT
GATCTCCTTCTGTCCAGACTGTGGCAAAAGTATCCAAGCGGCATTCAAATTCTGCCCCTA
CTGTGGAAATTCTTTGCCTGTAGAGGAGCATGTAGGGTCCCAGACCTTTGTCAATCCACA
TGTGTCATCCTTCCAAGGCTCAAAGAGAGGGCTGAACTCCAGTTTTGAAACCTCTCCTAA
GAAAGTGAAATGGTCCAGCACCGTCACCTCTCCCCGATTATCCCTCTTCTCAGATGGTGA
CAGTTCTGAGTCTGAAGATACTCTGAGTTCCTCTGAGAGATCCAAAGGCTCCGGGAGCAG
ACCCCCAACCCCCAAAAGCAGCCCTCAGAAGACCAGGAAGAGCCCTCAGGTGACCAGGGG
TAGCCCTCAGAAGACCAGCTGTAGCCCTCAGAAGACCAGGCAGAGCCCTCAGACGCTGAA
GCGGAGCCGAGTGACCACCTCACTTGAAGCTTTGCCCACAGGGACAGTGCTGACAGACAA
GAGTGGGCGACAGTGGAAGCTGAAGTCCTTCCAGACCAGGGACAACCAGGGCATTCTCTA
TGAAGCTGCACCCACCTCCACCCTCACCTGTGACTCAGGACCACAGAAGCAAAAGTTCTC
ACTCAAACTGGATGCCAAGGATGGGCGCTTGTTCAATGAGCAGAACTTCTTCCAGCGGGC
CGCCAAGCCTCTGCAAGTCAACAAGTGGAAGAAGCTGTACTCGACCCCACTGCTGGCCAT
CCCTACCTGCATGGGTTTCGGTGTTCACCAGGACAAATACAGGTTCTTGGTGTTACCCAG
CCTGGGGAGGAGCCTTCAGTCGGCCCTGGATGTCAGCCCAAAGCATGTGCTGTCAGAGAG
GTCTGTGCTGCAGGTGGCCTGCCGGCTGCTGGATGCCCTGGAGTTCCTCCATGAGAATGA
GTATGTTCATGGAAATGTGACAGCTGAAAATATCTTTGTGGATCCAGAGGACCAGAGTCA
GGTGACTTTGGCAGGCTATGGCTTCGCCTTCCGCTATTGCCCAAGTGGCAAACACGTGGC
CTACGTGGAAGGCAGCAGGAGCCCTCACGAGGGGGACCTTGAGTTCATTAGCATGGACCT
GCACAAGGGATGCGGGCCCTCCCGCCGCAGCGACCTCCAGAGCCTGGGCTACTGCATGCT
GAAGTGGCTCTACGGGTTTCTGCCATGGACAAATTGCCTTCCCAACACTGAGGACATCAT
GAAGCAAAAACAGAAGTTTGTTGATAAGCCGGGGCCCTTCGTGGGACCCTGCGGTCACTG
GATCAGGCCCTCAGAGACCCTGCAGAAGTACCTGAAGGTGGTGATGGCCCTCACGTATGA
GGAGAAGCCGCCCTACGCCATGCTGAGGAACAACCTAGAAGCTTTGCTGCAGGATCTGCG
TGTGTCTCCATATGACCCCATTGGCCTCCCGATGGTGCCCTAGGTGGAATCCAGAACTTT
CCATTTGCAGTGTGCAACAGAAAAAAAAATGAAGCAATGTGACTCAAGGCCTGCTGTTTA
ATCACAGATAAGCTTCTAGAACAAGCCCTGGAATGTGCATTCCTGCCACTGGTTTCAGGA
TACTCATCAGTCCTGATTAGCCTCCGGAGGGCCCCAGTTTCCCTCCCGTGAATGTGAAGT
TCCCCATCTTGGTGGCCTGCCCTTCAGCCAGTGTCCTAGCAAAGCTGGATGGGGTTGGGC
CGGCCCACAGGGGGGACCCCTCCTACCCTTGACTCCTCTGTGCTTTGGTAATAAATTGTT
TTACCAGAG
>gi|7705993|gb|NP_057524.1|LOC51231 474 aa linear VRK3 for
vaccinia related kinase 3 [Homo sapiens].
MISFCPDCGKSIQAAFKFCPYCGNSLPVEEHVGSQTFVNPHVSSFQGSKRGLNSSFETSP
KKVKWSSTVTSPRLSLFSDGDSSESEDTLSSSERSKGSGSRPPTPKSSPQKTRKSPQVTR
GSPQKTSCSPQKTRQSPQTLKRSRVTTSLEALPTGTVLTDKSGRQWKLKSFQTRDNQGIL
YEAAPTSTLTCDSGPQKQKFSLKLDAKDGRLFNEQNFFQRAAKPLQVNKWKKLYSTPLLA
IPTCMGFGVHQDKYRFLVLPSLGRSLQSALDVSPKHVLSERSVLQVACRLLDALEFLHEN
EYVHGNVTAENIFVDPEDQSQVTIAGYGFAFRYCPSGKHVAYVEGSRSPHEGDLEFISMD
LHKGCGPSRRSDLQSLGYCMLKWLYGFLPWTNCLPNTEDIMKQKQKFVDKPGPFVGPCGH
WIRPSETLQKYLKVVMALTYEEKPPYAMLRNNLEALLQDLRVSPYDPIGLPMVP
>gi|27479296|gb|XM_114075.2|TCEA3 1543 bp mRNA Homo sapiens
transcription elongation factor A (SII), 3 (TCEA3), mRNA.
CGCCCCCGCCGGGCGTGTGTGTCGTGTGTGTTTGGGGCCCGCGCGGGTTGCGCGCCCTCC
GCCTTCGCGCCTCCTGCCCCCGAGGCCCTACTGCTGCCCCTGTGCCCCTCGCCCCGCCGG
GCGTCGCGGGCCAACATGGGCCAGGAAGAGGAGCTGCTGAGGATCGCCAAAAAGCTGGAG
AAGATGGTGGCCAGGAAGAACACGGAAGGGGCCCTGGACCTTCTGAAGAAGCTGCACAGC
TGCCAGATGTCCATCCAGCTACTACAGACAACCAGGATTGGAGTTGCTGTTAATGGGGTC
CGCAAGCACTGCTCAGACAAGGAGGTGGTGTCCTTGGCCAAAGTCCTTATCAAAAACTGG
AAGCGGCTGCTAGACTCCCCTGGACCCCCAAAAGGAGAAAAAGGAGAGGAAAGAGAAAAG
GCAAAGAAGAAGGAAAAAGGGCTTGAGTGTTCAGACTGGAAGCCAGAAGCAGGCCTTTCT
CCACCAAGGAAAAAACGAGAAGACCCCAAAACCAGGAGAGACTCTGTGGACTCCAAGTCT
TCTGCCTCCTCCTCTCCAAAAAGACCATCGGTGGAAAGATCAAACAGCAGCAAATCAAAA
GCGGAGAGCCCCAAAACACCTAGCAGCCCCTTGACCCCCACGTTTGCCTCTTCCATGTGT
CTCCTGGCCCCCTGCTATCTCACAGGGGACTCTGTCCGGGACAAGTGTGTGGAGATGCTG
TCAGCAGCCCTGAAGGCGGACGATGATTACAAGGACTATGGAGTCAACTGTGACAAGATG
GCATCAGAATCGAAAGATCATATCTACCAAGAGCTCAAGAGCACGGACATGAAGTACCGG
AACCGCGTGCGCAGCCGCATAAGCAACCTCAAGGACCCCAGGAACCCCGGCCTGCGGCGG
AACGTGCTCAGTGGGGCCATCTCCGCAGGGCTTATAGCCAAGATGACGGCAGAGGAAATG
GCCAGTGATGAACTGAGGGAGTTGAGGAATGCCATGACCCAGGAGGCCATCCGTGAGCAC
CAGATGGCCAAGACTGGCGGCACCACCACTGACCTCTTCCAGTGCAGCAAATGCAAGAAG
AAGAACTGCACCTATAACCAGGTGCAGACACGCAGTGCTGATGAGCCCATGACTACCTTT
GTCTTATGCAATGAATGTGGCAATCGCTGGAAGTTCTGCTGATGGAACAGCCAGCCATGA
ACAAGGTGAGGAAGAAGAAAGAGGAAGCGCTGAATTATCTGAACTGGAGAAGCAATAAAA
ATTAAAGTGAAGGAAAATACTGAACTCTGTCTGAGTGGGATGGTATGAGTTAGAGGAAGA
ATTCTCTTGCAAATTAATAATCGGTCATTAGAAACAATTGGTTAATGGGGGAGCCTAATT
GGAGAATGATGCTGAGAATTTGTATTGATGAACCTCTTTTAGAAACTGCAGAGGGCTGGG
CACGGTGGTTTATGGCTGTAATCTGCAAACTCTGGGAGGCTGAGGTGGGAGAATCGCTTA
ACCCCAGAAGTTTGAGTCCAGCCCAGGCAACACAGCAAGACCC
>gi|20473950|gb|XP_114075.1|TCEA3 348 aa linear similar to
Transcription elongation factor A protein 3 (Transcription
elongation factor S-II protein 3) (Transcription elongation
factor TFIIS.h) [Homo sapiens].
MGQEEELLRIAKKLEKMVARKNTEGALDLLKKLHSCQMSIQLLQTTRIGVAVNGVRKHCS
DKEVVSLAKVLIKNWKRLLDSPGPPKGEKGEEREKAKKKEKGLECSDWKPEAGLSPPRKK
REDPKTRRDSVDSKSSASSSPKRPSVERSNSSKSKAESPKTPSSPLTPTFASSMCLLAPC
YLTGDSVRDKCVEMLSAALKADDDUKDUGVNCDKMASEIEDHIYQELKSTDMKYRNRVRS
RISNLKDPRNPGLRRNVLSGAISAGLIAKMTAEEMASDELRELRNAMTQEAIREHQMAKT
GGTTTDLFQCSKCKKKNCTYNQVQTRSADEPMTTFVLCNECGNRWKFC
>gi|21314607|gb|NM_003342.2|UBE2G1 2430 bp mRNA Homo sapiens
ubiquitin-conjugating enzyme E2G 1 (UBC7 homolog, C.
elegans) (UBE2G1), mRNA.
ACCGGCAGCGAGGCGCCGCTCCCGCCGCCTCAGCCCGGCCTTCCTCGGCTCCGGCGCTCC
GGTCGCGGGGCCCGGGTTCCTCGGCACACCCCGCTCCAGCCGCCCCCAGAGCCTGTCCCC
AGCCCTTCGGAAGCCCCGGCGCCAGCCCGGGCCCTCGGCAGGGAGGATGACGGAGCTGCA
GTCGGCACTGCTACTGCGAAGACAGCTGGCAGAACTCAACAAAAATCCAGTGGAAGGCTT
TTCTGCAGGTTTAATAGATGACAATGATCTCTACCGATGGGAAGTCCTTATTATTGGCCC
TCCAGATACACTTTATGAAGGTGGTGTTTTTAAGGCTCATCTTACTTTCCCAAAAGATTA
TCCCCTCCGACCTCCTAAAATGAAATTCATTACAGAAATCTGGCACCCAAATGTTGATAA
AAATGGTGATGTGTGCATTTCTATTCTTCATGAGCCTGGGGAAGATAAGTATGGTTATGA
AAAGCCAGAGGAACGCTGGCTCCCTATCCACACTGTGGAAACCATCATGATTAGTGTCAT
TTCTATGCTGGCAGACCCTAATGGAGACTCACCTGCTAATGTTGATGCTGCGAAAGAATG
GAGGGAAGATAGAAATGGAGAATTTAAAAGAAAAGTTGCCCGCTGTGTAAGAAAAAGCCA
AGAGACTGCTTTTGAGTGACATTTATTTAGCAGCTAGTAACTTCACTTATTTCAGGGTCT
CCAATTGAGAAACATGGCACTGTTTTTCCTGCACTCTACCCACCTATTGCTGGACTTCTG
TTGTACAAGTTGGCAAACACTGGCTGGAACTGGGCTGCAATAAAACATGCCAGTTATCAA
TGCTGACAAGAGCCTAACAAGTGCCAACTTACAGATGATTACGCATTTTGAATTCTAATG
AACTGTTTTAACCTTCAGGAAGAATTGTAAAGACCTGTACATAGCACAACATGATCCGGA
TAATATATATACTGTTCATGTACATCCACAAATACACCTTGTACCAAATAATGCTTTCTT
GTAGTAGAATAAGAATCGTGTAAATTCTAAGAGATTTTAGCAGGTTTTCTTTCCTATTCA
TTGTTTCTTATCAGTTTAAAAGGATTCCTTTAAGCATGTCAGATGAAAAGCAATTAGGAT
TAAAAGTTTCCATTTAATTTCCCTTAAACCCTTGAGGCTTCATTAAACTCTTTTCACTTA
CTAAACTTTTGTATCTTCTTTGTTTTGACACACTCCCCTTTGCTTTTATCTCTTACCTGC
CAGAATGTTCTCAAATGATTTAGTTCAAATACTGAAATACTTAATGAGCAATTACTTGAT
TTTTAATGATGACTTCGAAGGAGTCATCACTAGGTGCTTTGTCCTTTTTGTATTCTAGTT
GCACCCACCTCTTGGATTGGATATAGCAATAACATTTATTGGCCGTTGTGAGCTCTTGAT
CCCAGTCATTACCCCTGAGAACTAAAAATAGATGGTTCTTAATTCAACTTACTGAAAATT
TCCCCAAACAATAGCAAATCTGACTTTTCCCTCTTCAGTTGCCTGGTATTAAGGTTGGAT
AAATGAAGCATGCACAGCTACAGGCTTTCTACTTAACTTCTGGGTTTGCTATTACAAATC
CTATTTACTCTCATACCCTTCTCCTTAGTCCTTCATATTTCTCTGCCTCTATTCTTCTAT
ACTGCAGATTTTTCTCACCTATTGTACAAAGAAATTGCGATGTATATTTTCATGTAATTT
GATTTTGGAATTCTGTCACCTTATGTAGTGAGTTCTTCCAAAATATAATTTTTTTTCAAT
AATTGTCAAGTTGTTGGCTTTTATTGTATTGAATGAAGGCTATAATACTGAGTGCCAGAG
AAGTGGTTTAGGAAAATCTCAGGTTGATTCCTTATGCAAATGAACTTTTAATACTTGAAA
ATCACATGGCCATGGCAGTATATGTATTTGGTTCTATCTAGATTCTTCTGTGAATCTAAA
AGCATTACAGGGGTAAATGCTTTGCTATTTGACGTATAGATCCCGTCACTAACAATAGTA
CACTTGGATGTGATTAATGTTTGAGCTTCAATATATTTCATATCATACAGTTTTCTAAAA
CAACTTCAGCAAATGGTAAAATGAACATGTGCAGTGTTAAAGGCAGGCCTTAGGCTCCTT
CATGTTTGTTGTGAGGTTGTGTGTGGGAAGTAGTCTTTGGCTTATAAGGGATAGAACTTG
AGACAGTAGCAGATGGGACATGGTGTTTGATTGTGAGAATCAGTGAGAATTCGTGCATCT
CTGCTCTGTGGGGTTTGGAGAAATGCTTTGGCAGAAGAGTGAAAGAACTCCTGCCAAGAG
CCCAGACCTCTACAAACGTTGTATGTCCTTTTTTAAGCAGAAATAAAATGGTTGAGGACG
AAAAAAAAAAAAAAAAAAAAAAAAAAAAAA
>gi|13489085|gb|NP_003333.1|UBE2G1 170 aa linear ubiquitin-
conjugating enzyme E2G 1 (UBC7 homolog, C. elegans);
Ubiquitin-conjugating enzyme E2G (UBC7, C. elegans, human
homolog of); ubiquitin-conjugating enzyme E2G (homologous to
C. elegans UBC7); ubiquitin-conjugating enzyme E2G 1
(homologous to C. elegans UBC7) [Homo sapiens].
MTELQSALLLRRQLAELNKNPVEGFSAGLIDDNDLYRWEVLIIGPPDTLYEGGVFKAHLT
FPKDYPLRPPKMKFITEIWHPNVDKNGDVCISILHEPGEDKYGYEKPEERWLPIHTVETI
MISVISMLADPNGDSPANVDAAKEWREDRNGEFKRKVARCVRKSQETAFE
>gi|21361498|gb|NM_015670.2|SENP3 2258 bp mRNA Homo sapiens
sentrin/SUMO-specific protease 3 (SENP3), mRNA.
GAAGCTTGAGGCCGGAGACGCCCGCCTTCGGGCCCGTCCGCCCGGCTTCCCCGCTCCCGG
GTACTGGAAGATGAAAGAGACTATACAAGGGACCGGGTCCTGGGGGCCTGAGCCTCCTGG
ACCCGGCATACCCCCAGCTTACTCAAGTCCCAGGCGGGAGCGTCTTCGTTGGCCCCCACC
TCCCAAACCCCGACTCAAGTCAGGTGGAGGGTTTGGGCCAGATCCTGGGTCAGGGACCAC
AGTGCCAGCCAGACGCCTCCCTGTCCCCCGACCCTCTTTTGATGCCTCAGCAAGTGAAGA
GGAGGAAGAAGAGGAGGAGGAGGAGGATGAAGATGAAGAGGAGGAAGTGGCAGCTTGGAG
GCTGCCCCCAAGATGGAGTCAGCTGGGAACCTCCCAGCGGCCCCGCCCTTCCCGCCCCAC
TCATCGAAAAACCTGCTCACAGCGCCGCCGCCGAGCCATGAGAGCCTTCCGGATGCTGCT
CTACTCAAAAAGCACCTCGCTGACATTCCACTGGAAGCTTTGGGGGCGCCACCGGGGCCG
GCGGCGGGGCCTCGCACACCCCAAGAACCATCTTTCACCCCAGCAAGGGGGTGCGACGCC
ACAGGTGCCATCCCCCTGTTGTCGTTTTGACTCCCCCCGGGGGCCACCTCCACCCCGGCT
GGGTCTGCTAGGTGCTCTCATGGCTGAGGATGGGGTGAGAGGGTCTCCACCAGTGCCCTC
TGGGCCCCCCATGGAGGAAGATGGACTCAGGTGGACTCCAAAGTCTCCTCTGGACCCTGA
CTCGGGCCTCCTTTCATGTACTCTGCCCAACGGTTTTGGGGGACAATCTGGGCCAGAAGG
GGAGCGCAGCTTGGCACCCCCTGATGCCAGCATCCTCATCAGCAATGTGTGCAGCATCGG
GGACCATGTGGCCCAGGAGCTTTTTCAGGGCTCAGATTTGGGCATGGCAGAAGAGGCAGA
GAGGCCTGGGGAGAAAGCCGGCCAGCACAGCCCCCTGCGAGAGGAGCATGTGACCTGCGT
ACAGAGCATCTTGGACGAATTCCTTCAAACGTATGGCAGCCTCATACCCCTCAGCACTGA
TGAGGTAGTAGAGAAGCTGGAGGACATTTTCCAGCAGGAGTTTTCCACCCCTTCCAGGAA
GGGCCTGGTGTTGCAGCTGATCCAGTCTTACCAGCGGATGCCAGGCAATGCCATGGTGAG
GGGCTTCCGAGTGGCTTATAAGCGGCACGTGCTGACCATGGATGACTTGGGGACCTTGTA
TGGACAGAACTGGCTCAATGACCAGGTGATGAACATGTATGGAGACCTGGTCATGGACAC
AGTCCCTGAAAAGGTGCATTTCTTCAATAGTTTCTTCTATGATAAACTCCGTACCAAGGG
TTATGATGGGGTGAAAAGGTGGACCAAAAACGTGGACATCTTCAATAAGGAGCTACTGCT
AATCCCCATCCACCTGGAGGTGCATTGGTCCCTCATCTCTGTTGATGTGAGGCGACGCAC
CATCACCTATTTTGACTCGCAGCGTACCCTAAACCGCCGCTGCCCTAAGCATATTGCCAA
GTATCTACAGGCAGAGGCGGTAAAGAAAGACCGACTGGATTTCCACCAGGGCTGGAAAGG
TTACTTCAAAATGAATGTGGCCAGGCAGAATAATGACAGTGACTGTGGTGCTTTTGTGTT
GCAGTACTGCAAGCATCTGGCCCTGTCTCAGCCATTCAGCTTCACCCAGCAGGACATGCC
CAAACTTCGTCGGCAGATCTACAAGGAGCTGTGTCACTGCAAACTCACTGTGTGAGCCTC
GTACCCCAGACCCCAAGCCCATAAATGGGAAGGGAGACATGGGAGTCCCTTCCCAAGAAA
CTCCAGTTCCTTTCCTCTCTTGCCTCTTCCCACTCACTTCCCTTTGGTTTTTCATATTTA
AATGTTTCAATTTCTGTATTTTTTTTTCTTTGAGAGAATACTTGTTGATTTCTGATGTGC
AGGGGGTGGCTACAGAAAAGCCCCTTTCTTCCTCTGTTTGCAGGGGAGTGTGGCCCTGTG
GCCTGGGTGGAGCAGTCATCCTCCCCCTTCCCCGTGCAGGGAGCAGGAAATCAGTGCTGG
GGGTGGTGGGCGGACAATAGGATCACTGCCTGCCAGATCTTCAAACTTTTATATATATAT
ATATATATATATATATATATATAAAAATATATAAATGCCACGGTCCTGCTCTGGTCAATA
AAGGATCCTTTGTTGATACGTAAAAAAAAAAAAAAAAA
>gi|21361499|gb|NP_056485.2|SENP3 574 aa linear sentrin/
SUMO-specific protease 3 [Homo sapiens].
MKETIQGTGSWGPEPPGPGIPPAYSSPRRERLRWPPPPKPRLKSGGGFGPDPGSGTTVPA
RRLPVPRPSFDASASEEEEEEEEEEDEDEEEEVAAWRLPPRWSQLGTSQRPRPSRPTHRK
TCSQRRRRAMRAFRMLLYSKSTSLTFHWKLWGRHRGRRRGLAHPKNHLSPQQGGATPQVP
SPCCRFDSPRGPPPPRLGLLGALMAEDGVRGSPPVPSGPPMEEDGLRWTPKSPLDPDSGL
LSCTLPNGFGGQSGPEGERSLAPPDASILISNVCSIGDHVAQELFQGSDLGMAEEABRPG
EKAGQHSPLREEHVTCVQSILDEFLQTYGSLIPLSTDEVVEKLEDIFQQEFSTPSRKGLV
LQLIQSYQRMPGNAMVRGFRVAYKRHVLTMDDLGTLYGQNWLNDQVMNMYGDLVMDTVPE
KVHFFNSFFYDKLRTKGYDGVKRWTKNVDIFNKELLLIPIHLEVHWSLISVDVRRRTITY
FDSQRTLNRRCPKHIAKYLQAEAVKKDRLDFHQGWKGYFKMNVARQNNDSDCGAFVLQYC
KHLALSQPFSFTQQDMPKLRRQIYKELCHCKLTV
>gi|5803166|gb|NM_006802.1|SF3A3 2733 bp mRNA Homo sapiens
splicing factor 3a, subunit 3, 60kDa (SF3A3), mRNA.
AAGGGAAGATGGAGACAATACTGGAGCAGCAGCGGCGCTATCATGAGGAGAAGGAACGGC
TCATGGACGTCATGGCTAAAGAGATGCTCACCAAGAAGTCCACGCTCCGGGACCAGATCA
ATTCTGATCACCGCACTCGGGCCATGCAAGATAGGTATATGGAGGTCAGTGGGAACCTGA
GGGATTTGTATGATGATAAGGATGGATTACGAAAGGAGGAGCTCAATGCCATTTCAGGAC
CCAATGAGTTTGCTGAATTCTATAATAGACTCAAGCAAATAAAGGAATTCCACCGGAAGC
ACCCAAATGAGATCTGTGTGCCAATGTCAGTGGAATTTGAGGAACTCCTGAAGGCTCGAG
AGAATCCAAGTGAAGAGGCACAAAACTTGGTGGAGTTCACAGATGAGGAGGGATATGGTC
GTTATCTCGATCTCCATGACTGTTACCTCAAGTACATTAACCTGAAGGCATCTGAGAAGC
TGGATTATATCACATACCTGTCCATCTTTGACCAATTATTTGACATTCCTAAAGAAAGGA
AGAATGCAGAGTATAAGAGATACCTAGAGATGCTGCTTGAGTACCTTCAGGATTACACAG
ATAGAGTGAAGCCTCTCCAAGATCAGAATGAACTTTTTGGGAAGATTCAGGCTGAGTTTG
AGAAGAAATGGGAGAATGGGACCTTTCCTGGATGGCCGAAAGAGACAAGCAGTGCCCTGA
CCCATGCTGGAGCCCATCTTGACCTCTCTGCATTCTCCTCCTGGGAGGAGTTGGCTTCTC
TGGGTTTGGACAGATTGAAATCTGCTCTCTTAGCTTTAGGCTTGAAATGTGGCGGGACCC
TAGAAGAGCGAGCCCAGAGACTATTCAGTACCAAAGGAAAGTCCCTGGAGTCACTTGATA
CCTCTTTGTTTGCCAAAAATCCCAAGTCAAAGGGCACCAAGCGAGACACTGAAAGGAACA
AAGACATTGCTTTTCTAGAAGCCCAGATCTATGAATATGTAGAGATTCTCGGGGAACAGC
GACATCTCACTCATGAAAATGTACAGCGCAAGCAAGCCAGGACAGGAGAAGAGCGAGAAG
AAGAGGAAGAAGAGCAGATCAGTGAGAGTGAGAGTGAAGATGAAGAGAACGAGATCATTT
ACAACCCCAAAAACCTGCCACTTGGCTGGGATGGCAAACCTATTCCCTACTGGCTGTATA
AGCTTCATGGCCTAAATATCAACTACAACTGTGAGATTTGTGGAAACTACACCTACCGAG
GGCCCAAAGCCTTCCAGCGACACTTTGCTGAATGGCGTCATGCTCATGGCATGAGGTGTT
TGGGCATCCCAAATACTGCTCACTTTGCTAATGTGACACAGATTGAAGATGCTGTCTCCT
TGTGGGCCAAACTGAAATTGCAGAAGGCTTCAGAACGATGGCAGCCTGACACTGAGGAAG
AATATGAAGACTCAAGTGGGAATGTTGTGAATAAGAAGACATACGAGGATCTGAAAAGAC
AAGGACTGCTCTAGTGTTGAGGGATGTAGCTCAGCTTTTGGGCTAGCCCAGGCTTCCCTA
AGATCTGCTTTTTCTATTTCTCCCAACCAAATCCTCTTAAAGACCCTTTGCTATGTAGTC
TCATGGTCTAGCATGCATCTTGTAGAAACAAGGCATGCTGGCAGATTGCAGGGTTGAGAT
GTGTTTTATCTGTTTTATATTTTAAAAGATTCTGCCAGAAAATAAAACCAGACCTTGTTC
TAAAGCCCAGGGTTATGGACCAACTCAGTGCTTCAGGTCTTAATGCCTCCATACCTCTTC
CTCACCAACTTTACTAGTAGCTGAGATTTAATGGGCACCTATTATGCTACATATCATGTT
AGGTAAATCTGACCTGACCTCTTTCCCCACCCTCCTTTGTTGCTGCTTCCCTGAATGAGT
ATTACCCCAGGATGAGGTCTGCCATCAGCTTAGTTAGCCATTGATGCAAATACTAGGGAA
AGACTAGGAGGATGAGCCAGGGTTGCTACTAAGGACTAAGTGTCGCACCAAGGTTTGCCT
TTTGTATTTGCATAAAGAAAGGAGTTGGAGCTGGGTGCAGTGGCTTGTGCCTGTAGTCCC
AGCTACTTGGGAGGCTGAGGCAGGAGGGTTGCTTGAGACTAGCCTAGGTAACATAGTGAG
ACCCTGTCTCATTAAAAAAAAAAAAAAAAGGCATGGTGGCACGCACTGTAGTCCCAGCTA
CTCAGGAGACTGAGGCTAGAAGATCCTTTGAACCTAGGAGTTTGAGACCAGCCTGGGCGA
TATAGTGAGGCCCCATCTCAAAAAAAAAAAAAAGCGGGGGGGGGGAGTTGGGCTGTGTTG
GAATGGGCCTGCAGCCCAACAAACAAGGGAACTAGGACCGACAGTGACTTCACCAGCTTG
CTAGGTCAGAATGAGAGACTGGTGGGTCTGTCTACCTGTTTCTTCTACAAGATCCCTATT
TGACTGTAAAAGTAGCTAATACTCACATGTTCTCCAATCCCAGGTAGCCATGGTAGAGTT
GGGTAGAGTTGAGCAGCCGCCCCAGGATCCAAATGTGGTGTCTGAAATGGAAAGAACTAA
GGCAACCAGGAAGGCACTGATCTGCCTTATAAGCACAGTCATCTGAAAGTCAGGCCTGCT
GCAGGACAGGATCCCCCAGAGACCCCATTTGCCTCTCAACACTCAGACCTTCAACTGTTT
TTTAATAAATCTACTTTTTAAAAAAAAAAAATA
>gi|5803167|gb|NP_006793.1|SF3A3 501 aa linear splicing
factor 3a, subunit 3, 60kDa; pre-mRNA splicing factor SF3a
(60kD) [Homo sapiens].
METILEQQRRYHEEKERLMDVMAKEMLTKKSTLRDQINSDHRTRAMQDRYMEVSGNLRDL
YDDKDGLRKEELNAISGPNEFAEFYNRLKQIKEFHRKHPNEICVPMSVEFEELLKARENP
SEEAQNLVEFTDEEGYGRYLDLHDCYLKYINLKASEKLDYITYLSIFDQLFDIPKERKNA
EYKRYLEMLLEYLQDYTDRVKPLQDQNELFGKIQAEFEKKWENGTFPGWPKETSSALTHA
GAHLDLSAFSSWEELASLGLDRLKSALLALGLKCGGTLEERAQRLFSTKGKSLESLDTSL
FAKNPKSKGTKRDTERNKDIAFLEAQIYEYVEILGEQRHLTHENVQRKQARTGEEREEEE
EEQISESESEDEENEIIYNPKNLPLGWDGKPIPYWLYKLHGLNINYNCEICGNYTYRGPK
AFQRHFAEWRHAHGMRCLGIPNTAEFANVTQIEDAVSLWAKLKLQKASERWQPDTEEEYE
DSSGNVVNKKTYEDLKRQGLL
>gi|28882054|gb|NM_005011.2|NRF1 2514 bp mRNA Homo sapiens
nuclear respiratory factor 1 (NRF1), mRNA.
GAGGCTGCGAGGAGCCGGCGCGGTCGCAGTCTCCACGGCGCAGGCCCACGGTAGCGCAGC
CGCTCTGAGTAGAACTTCATGGAGGAACACGGAGTGACCCAAACCGAACATATGGCTACC
ATAGAAGCACATGCAGTGGCCCAGCAAGTGCAGCAGGTCCATGTGGCTACTTACACCGAG
CATAGTATGCTGAGTGCTGATGAAGACTCGCCTTCTTCTCCCGAGGACACCTCTTACGAT
GACTCAGATATACTCAACTCCACAGCAGCTGATGAGGTGACAGCTCATCTGGCAGCTGCA
GGTCCTGTGGGAATGGCCGCTGCTGCTGCTGTGGCAACAGGAAAGAAACGGAAACGGCCT
CATGTATTTGAGTCTAATCCATCTATCCGGAAGAGGCAACAAACACGTTTGCTTCGGAAA
CTTCGAGCCACGTTAGATGAATATACTACTCGTGTGGGACAGCAAGCTATTGTCCTCTGT
ATCTCACCCTCCAAACCTAACCCTGTCTTTAAAGTGTTTGGTGCAGCACCTTTGGAGAAT
GTGGTGCGTAAGTACAAGAGCATGATCCTGGAAGACCTGGAGTCTGCTCTGGCAGAACAC
GCCCCTGCGCCACAGGAGGTTAACTCAGAACTGCCGCCTCTCACCATCGACGGAATTCCA
GTCTCTGTGGACAAAATGACCCAGGCCCAGCTTCGGGCATTTATCCCAGAGATGCTCAAG
TACTCTACAGGTCGGGGAAAACCAGGCTGGGGGAAAGAAAGCTGCAAGCCCATCTGGTGG
CCTGAAGATATCCCCTGGGCAAATGTCCGGAGTGATGTCCGCACAGAAGAGCAAAAGCAG
AGGGTTTCATGGACCCAGGCACTACGGACCATAGTTAAAAACTGTTATAAACAGCATGGG
CGGGAAGACCTTTTGTATGCCTTTGAAGATCAGCAAACGCAAACACAGGCCACAGCCACA
CATAGTATAGCTCATCTTGTACCATCACAGACTGTAGTCCAGACTTTTAGTAACCCTGAT
GGCACTGTCTCACTTATCCAGGTTGGTACGGGGGCAACAGTAGCCACATTGGCTGATGCT
TCAGAATTGCCAACCACGGTCACCGTTGCCCAAGTGAATTATTCTGCCGTGGCTGATGGA
GAGGTGGAACAAAATTGGGCCACGTTACAGGGAGGTGAGATGACCATCCAGACGACGCAA
GCATCAGAGGCCACCCAGGCGGTGGCATCGTTGGCAGAGGCCGCAGTGGCAGCTTCTCAG
GAGATGCAGCAGGGAGCTACAGTCACTATGGCGCTTAACAGCGAAGCTGCCGCCCATGCT
GTCGCCACCCTGGCTGAGGCCACCTTACAAGGTGGGGGACAGATCGTCTTGTCTGGGGAA
ACCGCAGCAGCCGTCGGAGCACTTACTGGAGTCCAAGATGCTAATGGCCTCTTTATGGCA
GATCGTGCAGGTCGCAAGTGGATCCTGACTGACAAAGCCACAGGCCTGGTCCAGATCCCT
GTGAGCATGTACCAGACTGTGGTGACCAGCCTCGCCCAGGGCAACGGACCAGTGCAGGTG
GCCATGGCCCCTGTGACCACCAGGATATCAGACAGCGCAGTCACCATGGACGGCCAAGCT
GTGGAGGTGGTGACATTGGAACAGTGACATACAGCCATATTATGGCATCGTTTTCTAGTC
TACTTCAAAATTTTTTACACGTTTGCAGAGGTGCAATCAAATGGAATTAAGTCTCTCGAC
TTTGGAAGGAAAGTTTTGTTAACCTTTTTTTTTTTAAAAGGAAGAAAGCGGATTTTGGAA
TTGCATTTTTTAAAGCACCACTCTTGATTTTCTGGGATTGGTGAAGAAACTGCATTGTCA
ATTTCACTGTCCCAAAAAAGCCAAATTGTGGCAGGACTTCTTTCTGCGGAAATGTGTGTG
TATACTTATGTGTGTGTATGTGTGAGTGTGAATATATGTATATGTGTACATATGGACATA
CACATTTACATATATATAAAGTATATATATACATATATATATATATATGTATGAAACCCG
CATGGAATTATCTGTATGAAATCAAGGTGCGCTGTGGAAACAATAATTCACCCAGTTTAG
TGGGTGGTAGGGTACGTGGCCAGACACAGTCACCCAGTTTTTGTTCATACCAGGGTCATG
CGTTGAGCTACTGACAAACTCAGGCGGAGGTGACCATGCCCTTCACCAAAGCTGCCTCCC
AGTGGCCACACAGAACTCTCCCTGCTGGACTCACCTGAGGAAAGAGGCTCCAGCATGGGG
TGGGTCAGAGATGTGCTTGCAAGGTCCAGGGACTGCGTGGTCTGCCAGCTGAGATGCTCC
TCGGGCTGGCCCAGGTGCTGACCTTGCCACAGGCAGATGAATGTCTTGAAAGCTCCCGGG
CCTCAGCCTCCCATCTCCTCTCCTTCCCAGGAATCCTTGATCTCATGACTATTAAAATGT
TGCTCTGGTTTTAAGGTCAAAAAAAAAAAAAAAAAAAAAAAAAAAAAAAAAAAA
>gi|28882055|gb|NP_005002.2|NRF1 522 aa linear nuclear
respiratory factor 1 [Homo sapiens].
MEEHGVTQTEHMATIEAHAVAQQVQQVHVATYTEHSMLSADEDSPSSPEDTSYDDSDILN
STAADEVTAHLAAAGPVGMAAAAAVATGKKRKRPHVFESNPSIRKRQQTRLLRKLRATLD
EYTTRVGQQAIVLCISPSKPNPVFKVFGAAPLENVVRKYKSMILEDLESALAEHAPAPQE
VNSELPPLTIDGIPVSVDKMTQAQLRAFIPEMLKYSTGRGRPGWGKESCKPIWWPEDIPW
ANVRSDVRTEEQKQRVSWTQALRTIVKNCYKQHGREDLLYAFEDQQTQTQATATHSIAHL
VPSQTVVQTFSNPDGTVSLIQVGTGATVATLADASELPTTVTVAQVNYSAVADGEVEQNW
ATLQGGEMTIQTTQASEATQAVASIAEAAVAASQEMQQGATVTMALNSEAAAHAVATLAE
ATLQGGGQIVLSGETAAAVGALTGVQDANGLFMADRAGRKWILTDKATGLVQIPVSMYQT
VVTSLAQGNGPVQVAMAPVTTRISDSAVTMDGQAVEVVTLEQ
>gi|6996000|gb|NM_001663.2|ARF6 1806 bp mRNA Homo sapiens
ADP-ribosylation factor 6 (ARF6), mRNA.
GGCCGGAGGGAGCCCGCGCTCGGGGCGGCGGCTGGAGGCAGCGCACCGAGTTCCCGCGAG
GATCCATGACCTGACGGGGCCCCGGAGCCGCGCTGCCTCTCGGGTGTCCTGGGTCGGTGG
GGAGCCCAGTGCTCGCAGGCCGGCGGGCGGGCCGGAGGGCTGCAGTCTCCCTCGCGGTGA
GAGGAAGGCGGAGGAGCGGGAACCGCGGCGGCGCTCGCGCGGCGCCTGCGGGGGGAAGGG
CAGTTCCGGGCCGGGCCGCGCCTCAGCAGGGCGGCGGCTCCCAGCGCAGTCTCAGGGCCC
GGGTGGCGGCGGCGACTGGAGAAATCAAGTTGTGCGGTCGGTGATGCCCGAGTGAGCGGG
GGGCCTGGGCCTCTGCCCTTAGGAGGCAACTCCCACGCAGGCCGCAAAGGGCTCTCGCGG
CCGAGAGGCTTCGTTTCGGTTTCGCGGCGGCGGCGGCGTTGTTGGCTGAGGGGACCCGGG
ACACCTGAATGCCCCCGGCCCCGGCTCCTCCGACGCGATGGGGAAGGTGCTATCCAAAAT
CTTCGGGAACAAGGAAATGCGGATCCTCATGTTGGGCCTGGACGCGGCCGGCAAGACAAC
AATCCTGTACAAGTTGAAGCTGGGCCAGTCGGTGACCACCATTCCCACTGTGGGTTTCAA
CGTGGAGACGGTGACTTACAAAAATGTCAAGTTCAACGTATGGGATGTGGGCGGCCAGGA
CAAGATCCGGCCGCTCTGGCGGCATTACTACACTGGGACCCAAGGTCTCATCTTCGTAGT
GGACTGCGCCGACCGCGACCGCATCGATGAGGCTCGCCAGGAGCTGCACCGCATTATCAA
TGACCGGGAGATGAGGGACGCCATAATCCTCATCTTCGCCAACAAGCAGGACCTGCCCGA
TGCCATGAAACCCCACGAGATCCAGGAGAAACTGGGCCTGACCCGGATTCGGGACAGGAA
CTGGTATGTGCAGCCCTCCTGTGCCACCTCAGGGGACGGACTCTATGAGGGGCTCACATG
GTTAACCTCTAACTACAAATCTTAATGAGCATTCTCCACCCATCCCCTGGAAGGAGAGAA
ATCAAAAACCCATTCATAGGATTATCGCCACCATCACCTCTTTCAATTGCCACTTTCTCT
TCTTTTGAATTTGAACTCTGGAGTTACTGTTCTACAGTTTGGCGGGGACGGGGCTTGGGG
GTTTTCTCTTTTGTTTGTTTCCCTTTCTTTTTCCTTTTTTTTTTTTTTTTTTTGTTGGCT
TTGCGTTAGGATGGCTCTGATCTGACATTTGACATGAACACAAAGTTGCCAAGATGCTCC
TTGTTGACTTCCAGCAGAATGGGAATGGGGGAAACACAGCAGTTCTTGGGTAAAAGTCCC
TTTGTAATAATAGGTTTGGGATTTTTTTATTTCGAGAGAATCTTTCATTTTCCTATGTAT
GCTTTTTTCCTTTTTTGCCCAGTTTCCTTATCACTTGCTGTAGATGGCTTATTTTGCATT
CATGCAGACTATGTTGCAAGTCTGTTTCATCTAGTAAACTGAAAATTATTGCTTAATCAA
ACTGCCGTTTGTCTTTTATATTTAAGGCCTTCCCCCCCCTTCCTTATGAGTTCTAACTTA
GTAATTTCAAATGTGACCTTTTATATCTAAGACCAGTATAGTAAACTTAGCCCACAGTGG
CAAATAATGAGTAATATTGTAATATGTTCCAGTTGCACCTCAGTATGTTAAACAGGTAAT
GTAAGAAGTTCTCTGAAATGTCAGCAAGTAAGTTCTGAAACACATCATGCATGAGTAGGA
ATAAAC
>gi|4502211|gb|NP_001654.1|ARF6 175 aa linear ADP-ribosyla-
tion factor 6 [Homo sapiens].
MGKVLSKIFGNKEMRILMLGLDAAGKTTILYKLKLGQSVTTIPTVGFNVETVTYKNVKFN
VWDVGGQDKIRPLWRHYYTGTQGLIFVVDCADRDRIDEARQELHRIINDREMRDAIILIF
ANKQDLPDAMKPHEIQEKLGLTRIRDRNWYVQPSCATSGDGLYEGLTWLTSNYKS
>gi|23510442|gb|NM_003809.2|TNFSF12 1407 bp mRNA Homo
sapiens tumor necrosis factor (ligand) superfamily, member
12 (TNFSF12), transcript variant 1, mRNA.
CTCTCCCCGGCCCGATCCGCCCGCCGGCTCCCCCTCCCCCGATCCCTCGGGTCCCGGGAT
GGGGGGGCGGTGAGGCAGGCACAGCCCCCCGCCCCCATGGCCGCCCGTCGGAGCCAGAGG
CGGAGGGGGCGCCGGGGGGAGCCGGGCACCGCCCTGCTGGTCCCGCTCGCGCTGGGCCTG
GGCCTGGCGCTGGCCTGCCTCGGCCTCCTGCTGGCCGTGGTCAGTTTGGGGAGCCGGGCA
TCGCTGTCCGCCCAGGAGCCTGCCCAGGAGGAGCTGGTGGCAGAGGAGGACCAGGACCCG
TCGGAACTGAATCCCCAGACAGAAGAAAGCCAGGATCCTGCGCCTTTCCTGAACCGACTA
GTTCGGCCTCGCAGAAGTGCACCTAAAGGCCGGAAAACACGGGCTCGAAGAGCGATCGCA
GCCCATTATGAAGTTCATCCACGACCTGGACAGGACGGAGCGCAGGCAGGTGTGGACGGG
ACAGTGAGTGGCTGGGAGGAAGCCAGAATCAACAGCTCCAGCCCTCTGCGCTACAACCGC
CAGATCGGGGAGTTTATAGTCACCCGGGCTGGGCTCTACTACCTGTACTGTCAGGTGCAC
TTTGATGAGGGGAAGGCTGTCTACCTGAAGCTGGACTTGCTGGTGGATGGTGTGCTGGCC
CTGCGCTGCCTGGAGGAATTCTCAGCCACTGCGGCGAGTTCCCTCGGGCCCCAGCTCCGC
CTCTGCCAGGTGTCTGGGCTGTTGGCCCTGCGGCCAGGGTCCTCCCTGCGGATCCGCACC
CTCCCCTGGGCCCATCTCAAGGCTGCCCCCTTCCTCACCTACTTCGGACTCTTCCAGGTT
CACTGAGGGGCCCTGGTCTCCCCGCAGTCGTCCCAGGCTGCCGGCTCCCCTCGACAGCTC
TCTGGGCACCCGGTCCCCTCTGCCCCACCCTCAGCCGCTCTTTGCTCCAGACCTGCCCCT
CCCTCTAGAGGCTGCCTGGGCCTGTTCACGTGTTTTCCATCCCACATAAATACAGTATTC
CCACTCTTATCTTACAACTCCCCCACCGCCCACTCTCCACCTCACTAGCTCCCCAATCCC
TGACCCTTTGAGGCCCCCAGTGATCTCGACTCCCCCCTGGCCACAGACCCCCAGGGCATT
GTGTTCACTGTACTCTGTGGGCAAGGATGGGTCCAGAAGACCCCACTTCAGGCACTAAGA
GGGGCTGGACCTGGCGGCAGGAAGCCAAAGAGACTGGGCCTAGGCCAGGAGTTCCCAAAT
GTGAGGGGCGAGAAACAAGACAAGCTCCTCCCTTGAGAATTCCCTGTGGATTTTTAAAAC
AGATATTATTTTTATTATTATTGTGACAAAATGTTGATAAATGGATATTAAATAGAATAA
GTCATAAAAAAAAAAAAAAAAAAAAAA
>gi|4507597|gb|NP_003800.1|TNFSF12 249 aa linear tumor
necrosis factor (ligand) superfamily, member 12 isoform 1
precursor; APO3/DR3 ligand; TNF-related WEAK inducer of
apoptosis [Homo sapiens].
MAARRSQRRRGRRGEPGTALLVPLALGLGLALACLGLLLAVVSLGSRASLSAQEPAQEEL
VAEEDQDPSELNPQTEESQDPAPFLNRLVRPRRSAPKGRKTRARRAIAAHYEVHPRPGQD
GAQAGVDGTVSGWEEARINSSSPLRYNRQIGEFIVTRAGLYYLYCQVHFDEGKAVYLKLD
LLVDGVLALRCLEEFSATAASSLGPQLRLCQVSGLLALRPGSSLRIRTLPWAHLKAAPFL
TYFGLFQVH
>gi|11496238|gb|NM_021975.1|RELA. 2444 bp mRNA Homo sapiens
v-rel reticuloendotheliosis viral oncogene homolog A,
nuclear factor of kappa light polypeptide gene enhancer in
B-cells 3, p65 (avian) (RELA), mRNA.
GGCACGAGGCGGGGCCGGGTCGCAGCTGGGCCCGCGGCATGGACGAACTGTTCCCCCTCA
TCTTCCCGGCAGAGCAGCCCAAGCAGCGGGGCATGCGCTTCCGCTACAAGTGCGAGGGGC
GCTCCGCGGGCAGCATCCCAGGCGAGAGGAGCACAGATACCACCAAGACCCACCCCACCA
TCAAGATCAATGGCTACACAGGACCAGGGACAGTGCGCATCTCCCTGGTCACCAAGGACC
CTCCTCACCGGCCTCACCCCCACGAGCTTGTAGGAAAGGACTGCCGGGATGGCTTCTATG
AGGCTGAGCTCTGCCCGGACCGCTGCATCCACAGTTTCCAGAACCTGGGAATCCAGTGTG
TGAAGAAGCGGGACCTGGAGCAGGCTATCAGTCAGCGCATCCAGACCAACAACAACCCCT
TCCAAGTTCCTATAGAAGAGCAGCGTGGGGACTACGACCTGAATGCTGTGCGGCTCTGCT
TCCAGGTGACAGTGCGGGACCCATCAGGCAGGCCCCTCCGCCTGCCGCCTGTCCTTTCTC
ATCCCATCTTTGACAATCGTGCCCCCAACACTGCCGAGCTCAAGATCTGCCGAGTGAACC
GAAACTCTGGCAGCTGCCTCGGTGGGGATGAGATCTTCCTACTGTGTGACAAGGTGCAGA
AAGAGGACATTGAGGTGTATTTCACGGGACCAGGCTGGGAGGCCCGAGGCTCCTTTTCGC
AAGCTGATGTGCACCGACAAGTGGCCATTGTGTTCCGGACCCCTCCCTACGCAGACCCCA
GCCTGCAGGCTCCTGTGCGTGTCTCCATGCAGCTGCGGCGGCCTTCCGACCGGGAGCTCA
GTGAGCCCATGGAATTCCAGTACCTGCCAGATACAGACGATCGTCACCGGATTGAGGAGA
AACGTAAAAGGACATATGAGACCTTCAAGAGCATCATGAAGAAGAGTCCTTTCAGCGGAC
CCACCGACCCCCGGCCTCCACCTCGACGCATTGCTGTGCCTTCCCGCAGCTCAGCTTCTG
TCCCCAAGCCAGCACCCCAGCCCTATCCCTTTACGTCATCCCTGAGCACCATCAACTATG
ATGAGTTTCCCACCATGGTGTTTCCTTCTGGGCAGATCAGCCAGGCCTCGGCCTTGGCCC
CGGCCCCTCCCCAAGTCCTGCCCCAGGCTCCAGCCCCTGCCCCTGCTCCAGCCATGGTAT
CAGCTCTGGCCCAGGCCCCAGCCCCTGTCCCAGTCCTAGCCCCAGGCCCTCCTCAGGCTG
TGGCCCCACCTGCCCCCAAGCCCACCCAGGCTGGGGAAGGAACGCTGTCAGAGGCCCTGC
TGCAGCTGCAGTTTGATGATGAAGACCTGGGGGCCTTGCTTGGCAACAGCACAGACCCAG
CTGTGTTCACAGACCTGGCATCCGTCGACAACTCCGAGTTTCAGCAGCTGCTGAACCAGG
GCATACCTGTGGCCCCCCACACAACTGAGCCCATGCTGATGGAGTACCCTGAGGCTATAA
CTCGCCTAGTGACAGCCCAGAGGCCCCCCGACCCAGCTCCTGCTCCACTGGGGGCCCCGG
GGCTCCCCAATGGCCTCCTTTCAGGAGATGAAGACTTCTCCTCCATTGCGGACATGGACT
TCTCAGCCCTGCTGAGTCAGATCAGCTCCTAAGGGGGTGACGCCTGCCCTCCCCAGAGCA
CTGGTTGCAGGGGATTGAAGCCCTCCAAAAGCACTTACGGATTCTGGTGGGGTGTGTTCC
AACTGCCCCCAACTTTGTGGATGTCTTCCTTGGAGGGGGGAGCCATATTTTATTCTTTTA
TTGTCAGTATCTGTATCTCTCTCTCTTTTTGGAGGTGCTTAAGCAGAAGCATTAACTTCT
CTGGAAAGGGGGGAGCTGGGGAAACTCAAACTTTTCCCCTGTCCTGATGGTCAGCTCCCT
TCTCTGTAGGGAACTGTGGGGTCCCCCATCCCCATCCTCCAGCTTCTGGTACTCTCCTAG
AGACAGAAGCAGGCTGGAGGTAAGGCCTTTGAGCCCACAAAGCCTTATCAAGTGTCTTCC
ATCATGGATTCATTACAGCTTAATCAAAATAACGCCCCAGATACCAGCCCCTGTATGGCA
CTGGCATTGTCCCTGTGCCTAACACCAGCGTTTGAGGGGCTGCCTTCCTGCCCTACAGAG
GTCTCTGCCGGCTCTTTCCTTGCTCAACCATGGCTGAAGGAAACAGTGCAACAGCACTGG
CTCTCTCCAGGATCCAGAAGGGGTTTGGTCTGGACTTCCTTGCTCTCCCCTCTTCTCAAG
TGCCTTAATAGTAGGGTAAGTTGTTAAGAGTGGGGGAGAGCAGGCTGGCAGCTCTCCAGT
CAGGAGGCATAGTTTTTAGTGAACAATCAAAGCACTTGGACTCTTGCTCTTTCTACTCTG
AACTAATAAAGCTGTTGCCAAGCTGGACGGCACGAGCTCGTGCC
>gi|11496239|gb|NP_068810.1|RELA 537 aa linear v-rel
reticuloendotheliosis viral oncogene homolog A, nuclear
factor of kappa light polypeptide gene enhancer in B-cells
3, p65; v-rel avian reticuloendotheliosis viral oncogene
homolog A (nuclear factor of kappa light polypeptide gene
enhancer in B-cells 3 (p65)) [Homo sapiens].
MDELFPLIFPAEQPKQRGMRFRYKCEGRSAGSIPGERSTDTTKTHPTIKINGYTGPGTVR
ISLVTKDPPHRPHPHELVGKDCRDGFYEAELCPDRCIHSFQNLGIQCVKKRDLEQAISQR
IQTNNNPFQVPIEEQRGDYDLNAVRLCFQVTVRDPSGRPLRLPPVLSHPIFDNRAPNTAE
LKICRVNRNSGSCLGGDEIFLLCDKVQKEDIEVYFTGPGWEARGSFSQADVHRQVAIVFR
TPPYADPSLQAPVRVSMQLRRPSDRELSEPMEFQYLPDTDDRHRIEEKRKRTYETFKSIM
KKSPFSGPTDPRPPPRRIAVPSRSSASVPKPAPQPYPFTSSLSTINYDEFPTMVFPSGQI
SQASALAPAPPQVLPQAPAPAPAPAMVSALAQAPAPVPVLAPGPPQAVAPPAPKPTQAGE
GTLSEALLQLQFDDEDLGALLGNSTDPAVFTDLASVDNSEFQQLLNQGIPVAPHTTEPML
MEYPEAITRLVTAQRPPDPAPAPLGAPGLPNGLLSGDEDFSSIADMDFSALLSQISS
>gi|23312372|gb|NM_001065.2|TNFRSF1A 2236 bp mRNA Homo
sapiens tumor necrosis factor receptor superfamily, member
1A (TNFRSF1A), mRNA.
GCTGTTGCAACACTGCCTCACTCTTCCCCTCCCACCTTCTCTCCCCTCCTCTCTGCTTTA
ATTTTCTCAGAATTCTCTGGACTGAGGCTCCAGTTCTGGCCTTTGGGGTTCAAGATCACT
GGGACCAGGCCGTGATCTCTATGCCCGAGTCTCAACCCTCAACTGTCACCCCAAGGCACT
TGGGACGTCCTGGACAGACCGAGTCCCGGGAAGCCCCAGCACTGCCGCTGCCACACTGCC
CTGAGCCCAAATGGGGGAGTGAGAGGCCATAGCTGTCTGGCATGGGCCTCTCCACCGTGC
CTGACCTGCTGCTGCCACTGGTGCTCCTGGAGCTGTTGGTGGGAATATACCCCTCAGGGG
TTATTGGACTGGTCCCTCACCTAGGGGACAGGGAGAAGAGAGATAGTGTGTGTCCCCAAG
GAAAATATATCCACCCTCAAAATAATTCGATTTGCTGTACCAAGTGCCACAAAGGAACCT
ACTTGTACAATGACTGTCCAGGCCCGGGGCAGGATACGGACTGCAGGGAGTGTGAGAGCG
GCTCCTTCACCGCTTCAGAAAACCACCTCAGACACTGCCTCAGCTGCTCCAAATGCCGAA
AGGAAATGGGTCAGGTGGAGATCTCTTCTTGCACAGTGGACCGGGACACCGTGTGTGGCT
GCAGGAAGAACCAGTACCGGCATTATTGGAGTGAAAACCTTTTCCAGTGCTTCAATTGCA
GCCTCTGCCTCAATGGGACCGTGCACCTCTCCTGCCAGGAGAAACAGAACACCGTGTGCA
CCTGCCATGCAGGTTTCTTTCTAAGAGAAAACGAGTGTGTCTCCTGTAGTAACTGTAAGA
AAAGCCTGGAGTGCACGAAGTTGTGCCTACCCCAGATTGAGAATGTTAAGGGCACTGAGG
ACTCAGGCACCACAGTGCTGTTGCCCCTGGTCATTTTCTTTGGTCTTTGCCTTTTATCCC
TCCTCTTCATTGGTTTAATGTATCGCTACCAACGGTGGAAGTCCAAGCTCTACTCCATTG
TTTGTGGGAAATCGACACCTGAAAAAGAGGGGGAGCTTGAAGGAACTACTACTAAGCCCC
TGGCCCCAAACCCAAGCTTCAGTCCCACTCCAGGCTTCACCCCCACCCTGGGCTTCAGTC
CCGTGCCCAGTTCCACCTTCACCTCCAGCTCCACCTATACCCCCGGTGACTGTCCCAACT
TTGCGGCTCCCCGCAGAGAGGTGGCACCACCCTATCAGGGGGCTGACCCCATCCTTGCGA
CAGCCCTCGCCTCCGACCCCATCCCCAACCCCCTTCAGAAGTGGGAGGACAGCGCCCACA
AGCCACAGAGCCTAGACACTGATGACCCCGCGACGCTGTACGCCGTGGTGGAGAACGTGC
CCCCGTTGCGCTGGAAGGAATTCGTGCGGCGCCTAGGGCTGAGCGACCACGAGATCGATC
GGCTGGAGCTGCAGAACGGGCGCTGCCTGCGCGAGGCGCAATACAGCATGCTGGCGACCT
GGAGGCGGCGCACGCCGCGGCGCGAGGCCACGCTGGAGCTGCTGGGACGCGTGCTCCGCG
ACATGGACCTGCTGGGCTGCCTGGAGGACATCGAGGAGGCGCTTTGCGGCCCCGCCGCCC
TCCCGCCCGCGCCCAGTCTTCTCAGATGAGGCTGCGCCCCTGCGGGCAGCTCTAAGGACC
GTCCTGCGAGATCGCCTTCCAACCCCACTTTTTTCTGGAAAGGAGGGGTCCTGCAGGGGC
AAGCAGGAGCTAGCAGCCGCCTACTTGGTGCTAACCCCTCGATGTACATAGCTTTTCTCA
GCTGCCTGCGCGCCGCCGACAGTCAGCGCTGTGCGCGCGGAGAGAGGTGCGCCGTGGGCT
CAAGAGCCTGAGTGGGTGGTTTGCGAGGATGAGGGACGCTATGCCTCATGCCCGTTTTGG
GTGTCCTCACCAGCAAGGCTGCTCGGGGGCCCCTGGTTCGTCCCTGAGCCTTTTTCACAG
TGCATAAGCAGTTTTTTTTGTTTTTGTTTTGTTTTGTTTTGTTTTTAAATCAATCATGTT
ACACTAATAGAAACTTGGCACTCCTGTGCCCTCTGCCTGGACAAGCACATAGCAAGCTGA
ACTGTCCTAAGGCAGGGGCGAGCACGGAACAATGGGGCCTTCAGCTGGAGCTGTGGACTT
TTGTACATACACTAAAATTCTGAAGTTAAAGCTCTGCTCTTGGAAAAAAAAAAAAAAAAA
AAAAAAAAAAAAAAAA
>gi|4507575|gb|NP_001056.1|TNFRSF1A 455 aa linear tumor
necrosis factor receptor 1 precursor; tumor necrosis factor
receptor type 1; tumor necrosis factor-alpha receptor; tumor
necrosis factor binding protein 1 [Homo sapiens].
MGLSTVPDLLLPLVLLELLVGIYPSGVIGLVPHLGDREKRDSVCPQGKYIHPQNNSICCT
KCHKGTYLYNDCPGPGQDTDCRECESGSFTASENHLRHCLSCSKCRKEMGQVEISSCTVD
RDTVCGCRKNQYRHYWSENLFQCFNCSLCLNGTVHLSCQEKQNTVCTCHAGFFLRENECV
SCSNCKKSLECTKLCLPQIENVKGTEDSGTTVLLPLVIFFGLCLLSLLFIGLMYRYQRWK
SKLYSIVCGKSTPEKEGELEGTTTKPLAPNPSFSPTPGFTPTLGFSPVPSSTFTSSSTYT
PGDCPNFAAPRREVAPPYQGADPILATALASDPIPNPLQKWEDSAHKPQSLDTDDPATLY
AVVENVPPLRWKEFVRRLGLSDHEIDRLELQNGRCLREAQYSMLATWRRRTPRREATLEL
LGRVLRDMDLLGCLEDIEEALCGPAALPPAPSLLR
>gi|4506738|gb|NM_003952.1|RPS6KB2 1735 bp mRNA Homo sapiens
ribosomal protein S6 kinase, 70kDa, polypeptide 2 (RPS6KB2),
mRNA.
AGAGACTCGTGCCGAATGGCACGAGGCCGACGGGCCCGCGGGGCCGGCGCCGCCATGGCG
GCCGTGTTTGATTTGGATTTGGAGACGGAGGAAGGCAGCGAGGGCGAGGGCGAGCCAGAG
CTCAGCCCCGCGGACGCATGTCCCCTTGCCGAGTTGAGGGCAGCTGGCCTAGAGCCTGTG
GGACACTATGAAGAGGTGGAGCTGACTGAGACCAGCGTGAACGTTGGCCCAGAGCGCATC
GGGCCCCACTGCTTTGAGCTGCTGCGTGTGCTGGGCAAGGGGGGCTATGGCAAGGTGTTC
CAGGTGCGAAAGGTGCAAGGCACCAACTTGGGCAAAATATATGCCATGAAAGTCCTAAGG
AAGGCCAAAATTGTGCGCAATGCCAAGGACACAGCACACACACGGGCTGAGCGGAACATT
CTAGAGTCAGTGAAGCACCCCTTTATTGTGGAACTGGCCTATGCCTTCCAGACTGGTGGC
AAACTCTACCTCATCCTTGAGTGCCTCAGTGGTGGCGAGCTCTTCACGCATCTGGAGCGA
GAGGGCATCTTCCTGGAAGATACGGCCTGCTTCTACCTGGCTGAGATCACGCTGGCCCTG
GGCCATCTCCACTCCCAGGGCATCATCTACCGGGACCTCAAGCCCGAGAACATCATGCTC
AGCAGCCAGGGCCACATCAAACTGACCGACTTTGGACTCTGCAAGGAGTCTATCCATGAG
GGCGCCGTCACTCACACCTTCTGCGGCACCATTGAGTACATGGCCCCTGAGATTCTGGTG
CGCAGTGGCCACAACCGGGCTGTGGACTGGTGGAGCCTGGGGGCCCTGATGTACGACATG
CTCACTGGATCGCCGCCCTTTACCGCAGAGAACCGGAAGAAAACCATGGATAAGATCATC
AGGGGCAAGCTGGCACTGCCCCCCTACCTCACCCCAGATGCCCGGGACCTTGTCAAAAAG
TTTCTGAAACGGAATCCCAGCCAGCGGATTGGGGGTGGCCCAGGGGATGCTGCTGATGTG
CAGAGACATCCCTTTTTCCGGCACATGAATTGGGACGACCTTCTGGCCTGGCGTGTGGAC
CCCCCTTTCAGGCCCTGTCTGCAGTCAGAGGAGGACGTGAGCCAGTTTGATACCCGCTTC
ACACGGCAGACGCCGGTGGACAGTCCTGATGACACAGCCCTCAGCGAGAGTGCCAACCAG
GCCTTCCTGGGCTTCACATACGTGGCGCCGTCTGTCCTGGACAGCATCAAGGAGGGCTTC
TCCTTCCAGCCCAAGCTGCGCTCACCCAGGCGCCTCAACAGTAGCCCCCGGGTCCCCGTC
AGCCCCCTCAAGTTCTCCCCTTTTGAGGGGTTTCGGCCCAGCCCCAGCCTGCCGGAGCCC
ACGGAGCTACCTCTACCTCCACTCCTGCCACCGCCGCCGCCCTCGACCACCGCCCCTCTC
CCCATCCGTCCCCCCTCAGGGACCAAGAAGTCCAAGAGGGGCCGTGGGCGTCCAGGGCGC
TAGGAAGCCGGGTGGGGGTGAGGGTAGCCCTTGAGCCCTGTCCCTGCGGCTGTGAGAGCA
GCAGGACCCTGGGCCAGTTCCAGAGACCTGGGGGTGTGTCTGGGGGTGGGGTGTGAGTGC
GTATGAAAGTGTGTGTCTGCTGGGGCAGCTGTGCCCCTGAATCATGGGCACGGAGGGCCG
CCCGCCACACCCCGCGCTCAACTGCTCCCGTGGAAGATTAAAGGGCTGAATCATG
>gi|4506739|gb|NP_003943.1|RPS6KB2 495 aa linear ribosomal
protein S6 kinase, 70kDa, polypeptide 2; ribosomal protein
S6 kinase, 70kD, polypeptide 2; p70 ribosomal S6 kinase beta
[Homo sapiens].
MARGRRARGAGAAMAAVFDLDLETEEGSEGEGEPELSPADACPLAELRAAGLEPVGHYEE
VELTETSVNVGPERIGPHCFELLRVLGKGGYGKVFQVRKVQGTNLGKIYAMKVLRKAKIV
RNAKDTAHTRAERNILESVKHPFIVELAYAFQTGGKLYLILECLSGGELFTHLEREGIFL
EDTACFYLAEITLALGHLHSQGIIYRDLKPENIMLSSQGHIKLTDFGLCKESIHEGAVTH
TFCGTIEYMAPEILVRSGHNRAVDWWSLGALMYDMLTGSPPFTAENRKKTMDKIIRGKLA
LPPYLTPDARDLVKKFLKRNPSQRIGGGPGDAADVQRHPFFRHMNWDDLLAWRVDPPFRP
CLQSEEDVSQFDTRFTRQTPVDSPDDTALSESANQAFLGFTYVAPSVLDSIKEGFSFQPK
LRSPRRLNSSPRVPVSPLKFSPFEGFRPSPSLPEPTELPLPPLLPPPPPSTTAPLPIRPP
SGTKKSKRGRGRPGR
>gi|11995473|gb|NM_019884.1|GSK3A 2169 bp mRNA Homo sapiens
glycogen synthase kinase 3 alpha (GSK3A), mRNA.
GCCAGAGCGGCGCGGCCTGGAAGAGGCCAGGGCCCGGGGGAGGCGACGGCAGCGGCGGCG
GCTGGGGCAGCCCGGGCAGCCCGAGCCCCGCAGCCTGGGCCTGTGCTCGGCGCCATGAGC
GGCGGCGGGCCTTCGGGAGGCGGCCCTGGGGGCTCGGGCAGGGCGCGGACTAGCTCGTTC
GCGGAGCCCGGCGGCGGAGGCGGAGGAGGCGGCGGCGGCCCCGGAGGCTCGGCCTCCGGC
CCAGGCGGCACCGGCGGCGGAAAGGCATCTGTCGGGGCCATGGGTGGGGGCGTCGGGGCC
TCGAGCTCCGGGGGTGGACCCGGCGGCAGCGGCGGAGGAGGCAGCGGAGGCCCCGGCGCA
GGCACTAGCTTCCCGCCGCCCGGGGTGAAGCTGGGCCGTGACAGCGGGAAGGTGACCACA
GTCGTAGCCACTCTAGGCCAAGGCCCAGAGCGCTCCCAAGAAGTGGCTTACACGGACATC
AAAGTGATTGGCAATGGCTCATTTGGGGTCGTGTACCAGGCACGGCTGGCAGAGACCAGG
GAACTAGTCGCCATCAAGAAGGTTCTCCAGGACAAGAGGTTCAAGAACCGAGAGCTGCAG
ATCATGCGTAAGCTGGACCACTGCAATATTGTGAGGCTGAGATACTTTTTCTACTCCAGT
GGCGAGAAGAAAGACGAGCTTTACCTAAATCTGGTGCTGGAATATGTGCCCGAGACAGTG
TACCGGGTGGCCCGCCACTTCACCAAGGCCAAGTTGACCATCCCTATCCTCTATGTCAAG
GTGTACATGTACCAGCTCTTCCGCAGCTTGGCCTACATCCACTCCCAGGGCGTGTGTCAC
CGCGACATCAAGCCCCAGAACCTGCTGGTGGACCCTGACACTGCTGTCCTCAAGCTCTGC
GATTTTGGCAGTGCAAAGCAGTTGGTCCGAGGGGAGCCCAATGTCTCCTACATCTGTTCT
CGCTACTACCGGGCCCCAGAGCTCATCTTTGGAGCCACTGATTACACCTCATCCATCGAT
GTTTGGTCAGCTGGCTGTGTACTGGCAGAGCTCCTCTTGGGCCAGCCCATCTTCCCTGGG
GACAGTGGGGTGGACCAGCTGGTGGAGATCATCAAGGTGCTGGGAACACCAACCCGGGAA
CAAATCCGAGAGATGAACCCCAACTACACGGAGTTCAAGTTCCCTCAGATTAAAGCTCAC
CCCTGGACAAAGGTGTTCAAATCTCGAACGCCGCCAGAGGCCATCGCGCTCTGCTCTAGC
CTGCTGGAGTACACCCCATCCTCAAGGCTCTCCCCACTAGAGGCCTGTGCGCACAGCTTC
TTTGATGAACTGCGATGTCTGGGAACCCAGCTGCCTAACAACCGCCCACTTCCCCCTCTC
TTCAACTTCAGTGCTGGTGAACTCTCCATCCAACCGTCTCTCAACGCCATTCTCATCCCT
CCTCACTTGAGGTCCCCCAGCGGCACTACCACCCTCACCCCGTCCTCACAAGCTTTAACT
GAGACTCCGACCAGCTCAGACTGGCAGTCGACCGATGCCACACCTACCCTCACTAACTCC
TCCTGAGGGCCCCACCAAGCACCCTTCCACTTCCATCTGGGAGCCCCAAGAGGGCGTGGG
AAGGGGGGCCATAGCCCATCAAGCTCCTGCCCTGGCTGGGCCCCTAGACTAGAGGGCAGA
GGTAAATGAGTCCCTGTCCCCACCTCCAGTCCCTCCCTCACCAGCCTCACCCCTGTGGTG
GGCTTTTTAAGAGGATTTTAACTGGTTGTGGGGAGGGAAGAGAAGGACAGGGTGTTGGGG
GGATGAGGACCTCCTACCCCCTTGGCCCCCTCCCCTCCCCCAGACCTCCACCTCCTCCAG
ACCCCCTCCCCTCCTGTGTCCCTTGTAAATAGAACCAGCCCAGCCCGTCTCCTCTTCCCT
TCCCTGGCCCCCGGGTGTAAATAGATTGTTATAATTTTTTTCTTAAAGAAAACGTCGATT
CGCACCGTCCAACCTGCCCCGCCCCTCCTACAGCTGTAACTCCCCTCCTGTCCTCTGCCC
CCAAGGTCTACTCCCTCCTCACCCCACCCTGGAGGGCCAGGGGAGTGGAGAGAGCTCCTG
ATGTCTTAGTTTCCACAGTAAGGTTTGCCTGTGTACAGACCTCCGTTCAATAAATTATTG
GCATGAAAA
>gi|11995474|gb|NP_063937.1|GSK3A 483 aa linear glycogen
synthase kinase 3 alpha [Homo sapiens].
MSGGGPSGGGPGGSGRARTSSFAEPGGGGGGGGGGPGGSASGPGGTGGGKASVGAMGGGV
GASSSGGGPGGSGGGGSGGPGAGTSFPPPGVKLGRDSGKVTTVVATLGQGPERSQEVAYT
DIKVIGNGSFGVVYQARLAETRELVAIKKVLQDKRFKNRELQIMRKLDHCNIVRLRYFFY
SSGEKKDELYLNLVLEYVPETVYRVARHFTKAKLTIPILYVKVYMYQLFRSLAYIHSQGV
CHRDIKPQNLLVDPDTAVLKLCDFGSAKQLVRGEPNVSYICSRYYRAPELIFGATDYTSS
IDVWSAGCVLAELLLGQPIFPGDSGVDQLVEIIKVLGTPTREQIREMNPNYTEFKFPQIK
AHPWTKVFKSRTPPEAIALCSSLLEYTPSSRLSPLEACAHSFFDELRCLGTQLPNNRPLP
PLFNFSAGELSIQPSLNAILIPPHLRSPSGTTTLTPSSQALTETPTSSDWQSTDATPTLT
NSS
>gi|7019350|gb|NM_013246.1|CLC 1689 bp mRNA Homo sapiens
cardiotrophin-like cytokine (CLC), mRNA.
GCCTCCGGGAGAGGAGCCGCACCCGGCCGGCCCGGCCCCAGCCCCATGGACCTCCGAGCA
GGGGACTCGTGGGGGATGTTAGCGTGCCTGTGCACGGTGCTCTGGCACCTCCCTGCAGTG
CCAGCTCTCAATCGCACAGGGGACCCAGGGCCTGGCCCCTCCATCCAGAAAACCTATGAC
CTCACCCGCTACCTGGAGCACCAACTCCGCAGCTTGGCTGGGACCTATCTGAACTACCTG
GGCCCCCCTTTCAACGAGCCAGACTTCAACCCTCCCCGCCTGGGGGCAGAGACTCTGCCC
AGGGCCACTGTTGACTTGGAGGTGTGGCGAAGCCTCAATGACAAACTGCGGCTGACCCAG
AACTACGAGGCCTACAGCCACCTTCTGTGTTACTTGCGTGGCCTCAACCGTCAGGCTGCC
ACTGCTGAGCTGCGCCGCAGCCTGGCCCACTTCTGCACCAGCCTCCAGGGCCTGCTGGGC
AGCATTGCGGGCGTCATGGCAGCTCTGGGCTACCCACTGCCCCAGCCGCTGCCTGGGACT
GAACCCACTTGGACTCCTGGCCCTGCCCACAGTGACTTCCTCCAGAAGATGGACGACTTC
TGGCTGCTGAAGGAGCTGCAGACCTGGCTGTGGCGCTCGGCCAAGGACTTCAACCGGCTC
AAGAAGAAGATGCAGCCTCCAGCAGCTGCAGTCACCCTGCACCTGGGGGCTCATGGCTTC
TGACTTCTGACCTTCTCCTCTTCGCTCCCCCTTCAAACCCTGCTCCCACTTTGTGAGAGC
CAGCCCTGTATGCCAACACCTGTTGAGCCAGGAGACAGAAGCTGTGAGCCTCTGGCCCTT
TCCTGGACCGGCTGGGCGTGTGATGCGATCAGCCCTGTCTCCTCCCCACCTCCCAAAGGT
CTACCGAGCTGGGGAGGAGGTACAGTAGGCCCTGTCCTGTCCTGTTTCTACAGGAAGTCA
TGCTCGAGGGAGTGTGAAGTGGTTCAGGTTGGTGCAGAGGCGCTCATGGCCTCCTGCTTC
TTGCCTACCACTTGGCCAGTGCCCACCCAGCCCCTCAGGTGGCACATCTGGAGGGCAGGG
GTTGAGGGGCCACCACCACACATGCCTTTCTGGGGTGAAGCCCTTTGGCTGCCCCACTCT
CCTTGGATGGGTGTTGCTCCCTTATCCCCAAATCACTCTATACATCCAATTCAGGAAACA
AACATGGTGGCAATTCTACACAAAAAGAGATGAGATTAACAGTGCAGGGTTGGGGTCTGC
ATTGGAGGTGCCCTATAAACCAGAAGAGAAAATACTGAAAGCACAGGGGCAGGGACAGAC
CAGACCAGACCCAGGAGTCTCCAAAGCACAGAGTGGCAAACAAAACCCGAGCTGAGCATC
AGGACCTTGCCTCGAATTGTCTTCCAGTATTACGGTGCCTCTTCTCTGCCCCCTTTCCCA
GGGTATCTGTGGGTTGCCAGGCTGGGGAGGGCAACCATAGCCACACCACAGGATTTCCTG
AAAGTTTACAATGCAGTAGCATTTTGGGGTGTAGGGTGGCAGCTCCCCAAGGCCCTGCCC
CCCAGCCCCACCCACTCATGACTCTAAGTGTGTTGTATTAATATTTATTTATTTGGAGAT
GTTATTTATTAGATGATATTTATTGCAGAATTTCTATTCTTGTATTAACAAATAAAATGC
TTGCCCCAG
>gi|7019351|gb|NP_037378.1|CLC 225 aa linear cardiotrophin-
like cytokine; neurotrophin-1/B-cell stimulating factor-3
[Homo sapiens].
MDLRAGDSWGMLACLCTVLWHLPAVPALNRTGDPGPGPSIQKTYDLTRYLEHQLRSLAGT
YLNYLGPPFNEPDFNPPRLGAETLPRATVDLEVWRSLNDKLRLTQNYEAYSHLLCYLRGL
NRQAATAELRRSLAHFCTSLQGLLGSIAGVMAALGYPLPQPLPGTEPTWTPGPAHSDFLQ
KMDDFWLLKELQTWLWRSAKDFNRLKKKMQPPAAAVTLHLGAHGF
>gi|22068574|gb|XM_036493.3|ZNF213 3073 bp mRNA Homo sapiens
zinc finger protein 213 (ZNF213), mRNA.
GGCCTCTGGCCGCCTGGCTCCAACATCAAGCACCGGGCTCCGAGTGGCCGGGATCAGCGC
CCCGAGGCAGAGGCCGGAGGGCGCGCGCACTGCTAGGAAGTGCTGGTCCCCCGCGCCGCT
CTGCCAGCTTGGTCCCCCGGCAGACGCCCCTGTACGATCGCCGCTCGCCCCGCGGGCGAG
GCTGCGGTGGACAGCGCGGGGCTCCGGCTGGCTCGCCTTCCCGCCTGCCGTGTCCTGCTG
AGCGACCCTGGAGTACACATCCAGATGCCAGCCCAGCTACCACAGGGGATCCCTCTGGGA
GACTGAAAGTACAGGTTCTGGGGCCCAGGTTGAAGCCGACCAACCCTGAGCCTCAGGCCA
GGGGAATGGCAGCCCCCTTGGAGGCCCAGGACCAGGCCCCTGGGGAGGGAGAAGGGCTTC
TGATTGTGAAAGTGGAAGATTCCTCCTGGGAACAGGAATCTGCCCAGCATGAGGATGGCA
GGGATTCCGAAGCCTGCCGCCAGCGCTTCCGGCAATTCTGCTACGGGGATGTGCATGGGC
CTCATGAGGCCTTCAGCCAGCTCTGGGAGCTCTGCTGCCGCTGGCTGCGGCCCGAGCTGC
GTACCAAGGAGCAGATCCTGGAGCTGCTGGTGCTGGAGCAGTTCCTGACAGTGCTGCCAG
GGGAGATCCAGGGCTGGGTGCGTGAGCAGCACCCGGGAAGCGGTGAGGAGGCTGTCGCCT
TGGTGGAGGACCTACAGAAGCAGCCAGTGAAAGCCTGGCGACAGGATGTGCCCTCGGAGG
AGGCGGAACCCGAGGCTGCAGGCCGGGGATCCCAGGCCACGGGGCCTCCCCCGACGGTGG
GGGCACGGAGGCGGCCGTCTGTTCCCCAGGAGCAGCACAGCCATAGCGCCCAGCCTCCTG
CTCTTCTTAAAGAGGGTCGTCCCGGAGAGACGACGGACACCTGCTTTGTCTCTGGGGTCC
ATGGACCTGTGGCATTGGGAGACATCCCATTCTATTTCTCCCGGGAAGAATGGGGCACCC
TGGACCCTGCTCAGCGGGATCTCTTCTGGGACATAAAGCGGGAGAACTCCCGGAACACCA
CCCTGGGTTTTGGGCTCAAAGGCCAAAGTGAGAAGTCCCTGCTGCAGGAGATGGTGCCGG
TGGTGCCAGGCCAGACAGGCAGCGACGTGACTGTGTCCTGGAGCCCCGAGGAGGCTGAGG
CCTGGGAGAGCGAGAACCGGCCGAGGGCGGCCCTGGGCCCAGTGGTGGGCGCGCGACGGG
GGCGGCCACCCACTCGCCGGCGCCAGTTCCGGGACCTGGCAGCCGAGAAGCCGCACAGCT
GCGGGCAGTGTGGAAAGCGCTTCCGCTGGGGCTCGGACCTGGCGCGGCACCAGCGCACGC
ACACGGGCGAGAAGCCACACAAGTGCCCTGAGTGCGACAAGAGCTTCCGCAGCTCCTCGG
ACCTGGTGCGCCACCAAGGCGTGCACACGGGCGAGAAGCCCTTCTCCTGTTCCGAGTGCG
GCAAGAGCTTCAGCCGCAGCGCCTACCTGGCCGACCACCAGCGCATACACACGGGCGAGA
AGCCTTTCGGCTGCAGCGACTGCGGCAAGAGCTTCTCGCTGCGCTCCTACCTGCTGGACC
ATCGGCGTGTGCACACCGGTGAGCGGCCCTTCGGCTGCGGAGAGTGCGACAAGAGCTTCA
AGCAGCGCGCGCACCTCATCGCGCATCAGAGCCTGCACGCCAAGATGGCCCAGCCCGTGG
GGTGAGCAGCTGGCTTGGCCGGAAACCCGGGGGAGGCCCAGCCACGGCACATCCTGCTTT
GTTCACCACTGGGACTCTCCTTCCATCTGTGGCCACCTCCCGGGCTGTCCGAGGGACCCC
AGGGTACCTCACACTCGGAGCTCGCCTGCCCTGCTTGGCTCTGAGGACCTGCCCAGCGCT
CAAAGGGAACGGAAGCCTTCCCCTCCCGCCCCCGATCTTGTCCTCTTTCCCCCTTCTGCG
CCTAGCGTTCCTCTTCCCCTCTAGTTTCCTGGAGCCCCAACACATTCCTGGCAGGGACAG
CAGGGTGGCAAGGACTCAGGTCTAGGTCCCTTCCCAGAAGCCCCCGAGCCTCATTTGACT
GTGTGGCTCTTTGGCCCCCACCCTGTGGGGTGGGTCCATGGGTCAGGCCTCTGCCCTACC
AACCTGTGCCTTTCAGTGGGCGTGGAGGACTGGCCTTGGCCCCCCAGGGGGCTGCTGGAC
TTTGGGAGAGACAGCCCACACCTGTGGGACCGCGGGTCTTAGTCACGGCGGCAGGGGCTT
TCTGGCCCCCTCCCACTCCCGTTTCCAGGCCATGACCACTCTGCCCTGTCCTGGCCATAC
GGACTCGGCCTGCCTTTGCCCTCGGCCTACTTGCCCTAGCATGAGGCTCTGAGAGCCACC
TGCCCACCAATCTGGTGAGGATAATGGTGGCTCCAGCGACAGGAGGCCAACCCTGGAGAC
CAAGAACAGGGCGCCTGGCTGCCATCTTTTCCTCCAGAGGTGGGGCTGCACCAGACTCAG
CACTAGCACTCCATCAGCACTAGCACCTCACTCCATCAGCACTAGCACCTCACTCCATCG
GCCCCGGCACCCTGCTCCATCGGCACTGGCGCCCTGCTCCATCGGCACTAATGCTCCACT
CGGCGCCCCACTCCATCGGCCCCGCTCCATCGGCACTAATGCCCCACTCGGCGCCCCACT
CCATCAGCACTAATGCTCCACTCCATTGGCACTAACGCCCCAACTCCAGCGGCACTAATG
ACCCGCTCCTTTGACATTGGTGCCCCACTCCATCAGCACTAACGCCCTGCTCCATCGGCA
CTGGTGTCCCACTCCATTGTCACTAACGTCCGGCTCCATCGGCACTACCACCCCGCTCCA
TCATCACTATGTCCAGCTCCGTCGGCACTACCACCCTGCTCCATCATCACTACGTCCAGC
TCCAACGGCACTGGTGCCCCATTCCATCGGCACTAACGCCCCGCTCCACCGGCACCAGTG
CCTCGCTCCATTGGCACCAACGCCCAGCTCCACCGGTACTGGCTCCCTGCTCCATCGGCA
CTAACGCCCTGCT
>gi|14777854|gb|XP_036493.1|ZNF213 459 aa linear similar to
Zinc finger protein 213 (Putative transcription factor CR53)
[Homo sapiens].
MAAPLEAQDQAPGEGEGLLIVKVEDSSWEQESAQHEDGRDSEACRQRFRQFCYGDVHGPH
EAFSQLWELCCRWLRPELRTKEQILELLVLEQFLTVLPGEIQGWVREQHPGSGEEAVALV
EDLQKQPVKAWRQDVPSEEAEPEAAGRGSQATGPPPTVGARRRPSVPQEQHSHSAQPPAL
LKEGRPGETTDTCFVSGVHGPVALGDIPFYFSREEWGTLDPAQRDLFWDIKRENSRNTTL
GFGLKGQSEKSLLQEMVPVVPGQTGSDVTVSWSPEEAEAWESENRPRAALGPVVGARRGR
PPTRRRQFRDLAAEKPHSCGQCGKRFRWGSDLARHQRTHTGEKPHKCPECDKSFRSSSDL
VRHQGVHTGEKPFSCSECGKSFSRSAYLADHQRIHTGEKPFGCSDCGKSFSLRSYLLDHR
RVHTGERPFGCGECDKSFKQRAHLIAHQSLHAKMAQPVG
>gi|21536281|gb|NM_003656.3|CAMK1 1501 bp mRNA Homo sapiens
calcium/calmodulin-dependent protein kinase I (CAMK1), mRNA.
GGAGAGAGCCGCCGAGCCGAGCCGAGCCCCAGCTCCAGCAAGAGCGCGGGCGGGTGGCCC
AGGCACGCAGCGGTGAGGACCGCGGCCACAGCTCGGCGCCAACCACCGCGGGCCTCCCAG
CCAGCCCCGCGGCGGGGCAGCCGCAGGAGCCCTGGCTGTGGTCGGGGGGCAGTGGGCCAT
GCTGGGGGCAGTGGAAGGCCCCAGGTGGAAGCAGGCGGAGGACATTAGAGACATCTACGA
CTTCCGAGATGTTCTGGGCACGGGGGCCTTCTCGGAGGTGATCCTGGCAGAAGATAAGAG
GACGCAGAAGCTGGTGGCCATCAAATGCATTGCCAAGGAGGCCCTGGAGGGCAAGGAAGG
CAGCATGGAGAATGAGATTGCTGTCCTGCACAAGATCAAGCACCCCAACATTGTAGCCCT
GGATGACATCTATGAGAGTGGGGGCCACCTCTACCTCATCATGCAGCTGGTGTCGGGTGG
GGAGCTCTTTGACCGTATTGTGGAAAAAGGCTTCTACACGGAGCGGGACGCCAGCCGCCT
CATCTTCCAGGTGCTGGATGCTGTGAAATACCTGCATGACCTGGGCATTGTACACCGGGA
TCTCAAGCCAGAGAATCTGCTGTACTACAGCCTGGATGAAGACTCCAAAATCATGATCTC
CGACTTTGGCCTCTCCAAGATGGAGGACCCGGGCAGTGTGCTCTCCACCGCCTGTGGAAC
TCCGGGATACGTGGCCCCTGAAGTCCTGGCCCAGAAGCCCTACAGCAAGGCTGTGGATTG
CTGGTCCATAGGTGTCATCGCCTACATCTTGCTCTGCGGTTACCCTCCCTTCTATGACGA
GAATGATGCCAAACTCTTTGAACAGATTTTGAAGGCCGAGTACGAGTTTGACTCTCCTTA
CTGGGACGACATCTCTGACTCTGCCAAAGATTTCATCCGGCACTTGATGGAGAAGGACCC
AGAGAAAAGATTCACCTGTGAGCAGGCCTTGCAGCACCCATGGATTGCAGGAGATACAGC
TCTAGATAAGAATATCCACCAGTCGGTGAGTGAGCAGATCAAGAAGAACTTTGCCAAGAG
CAAGTGGAAGCAAGCCTTCAATGCCACGGCTGTGGTGCGGCACATGAGGAAACTGCAGCT
GGGCACCAGCCAGGAGGGGCAGGGGCAGACGGCGAGCCATGGGGAGCTGCTGACACCAGT
GGCTGGGGGGCCGGCAGCTGGCTGTTGCTGTCGAGACTGCTGCGTGGAGCCGGGCACAGA
ACTGTCCCCCACACTGCCCCACCAGCTCTAGGGCCCTGGACCTCGGGTCATGATCCTCTG
CGTGGGAGGGCTTGGGGGCAGCCTGCTCCCCTTCCCTCCCTGAACCGGGAGTTTCTCTGC
CCTGTCCCCTCCTCACCTGCTTCCCTACCACTCCTCACTGCATTTTCCATACAAATGTTT
CTATTTTATTGTTCCTTCTTGTAATAAAGGGAAGATAAAACCAAAAAAAAAAAAAAAAAA
A
>gi|4502553|gb|NP_003647.1|CAMK1 370 aa linear calcium/
calmodulin-dependent protein kinase I [Homo sapiens].
MLGAVEGPRWKQAEDIRDIYDFRDVLGTGAFSEVILAEDKRTQKLVAIKCIAKEALEGKE
GSMENEIAVLHKIKHPNIVALDDIYESGGHLYLIMQLVSGGELFDRIVEKGFYTERDASR
LIFQVLDAVKYLHDLGIVHRDLKPENLLYYSLDEDSKIMISDFGLSKMEDPGSVLSTACG
TPGYVAPEVLAQKPYSKAVDCWSIGVIAYILLCGYPPFYDENDAKLFEQILKAEYEFDSP
YWDDISDSAKDFIRHLMEKDPEKRFTCEQALQHPWIAGDTALDKNIHQSVSEQIKKNFAK
SKWKQAFNATAVVRHMRKLQLGTSQEGQGQTASHGELLTPVAGGPAAGCCCRDCCVEPGT
ELSPTLPHQL
>gi|13186237|gb|NM_023107.1|FGFR1 2590 bp mRNA Homo sapiens
fibroblast growth factor receptor 1 (fms-related tyrosine
kinase 2, Pfeiffer syndrome) (FGFR1), transcript variant 5,
mRNA.
CCTCTTGCGGCCACAGGCGCGGCGTCCTCGGCGGCGGGCGGCAGCTAGCGGGAGCCGGGA
CGCCGGTGCAGCCGCAGCGCGCGGAGGAACCCGGGTGTGCCGGGAGCTGGGCGGCCACGT
CCGGACGGGACCGAGACCCCTCGTAGCGCATTGCGGCGACCTCGCCTTCCCCGGCCGCGA
GCGCGCCGCTGCTTGAAAAGCCGCGGAACCCAAGGACTTTTCTCCGGTCCGAGCTCGGGG
CGCCCCGCAGGCGCACGGTACCCGTGCTGCAGTCGGGCACGCCGCGGCGCCGGGGGCCTC
CGCAGGGCGATGGAGCCGGTCTGCAAGGAAAGTGAGGCGCCGCCGCTGCGTTCTGGAGGA
GGGGGGCACAAGGTCTGGAGACCCCGGGTGGCGGACGGGAGCCCTCCCCCCGCCCCGCCT
CCGGGGCACCAGCTCCGGCTCCATTGTTCCCGCCCGGGCTGGAGGCGCCGAGCACCGAGC
GCCGCCGGGAGTCGAGCGCCGGCCGCGGAGCTCTTGCGACCCCGCCAGGACCCGAACAGA
GCCCGGGGGCGGCGGGCCGGAGCCGGGGACGCGGGCACACGCCCGCTCGCACAAGCCACG
GCGGACTCTCCCGAGGCGGAACCTCCACGCCGAGCGAGGGTCAGTTTGAAAAGGAGGATC
GAGCTCACTGTGGAGTATCCATGGAGATGTGGAGCCTTGTCACCAACCTCTAACTGCAGA
ACTGGGATGTGGAGCTGGAAGTGCCTCCTCTTCTGGGCTGTGCTGGTCACAGCCACACTC
TGCACCGCTAGGCCGTCCCCGACCTTGCCTGAACAAGATGCTCTCCCCTCCTCGGAGGAT
GATGATGATGATGATGACTCCTCTTCAGAGGAGAAAGAAACAGATAACACCAAACCAAAC
CGTATGCCCGTAGCTCCATATTGGACATCCCCAGAAAAGATGGAAAAGAAATTGCATGCA
GTGCCGGCTGCCAAGACAGTGAAGTTCAAATGCCCTTCCAGTGGGACCCCAAACCCCACA
CTGCGCTGGTTGAAAAATGGCAAAGAATTCAAACCTGACCACAGAATTGGAGGCTACAAG
GTCCGTTATGCCACCTGGAGCATCATAATGGACTCTGTGGTGCCCTCTGACAAGGGCAAC
TACACCTGCATTGTGGAGAATGAGTACGGCAGCATCAACCACACATACCAGCTGGATGTC
GTGGAGCGGTCCCCTCACCGGCCCATCCTGCAAGCAGGGTTGCCCGCCAACAAAACAGTG
GCCCTGGGTAGCAACGTGGAGTTCATGTGTAAGGTGTACAGTGACCCGCAGCCGCACATC
CAGTGGCTAAAGCACATCGAGGTGAATGGGAGCAAGATTGGCCCAGACAACCTGCCTTAT
GTCCAGATCTTGAAGGTAATCATGGCACCAGTCTTCGTGGGCCAGTCTACTGGGAAGGAG
ACCACTGTCTCGGGGGCTCAAGTTCCTGTGGGCAGGCTCAGTTGCCCCCGAATGGGATCA
TTCCTCACGCTTCAGGCACACACACTCCATCTCAGTAGGGATCTAGCCACATCCCCCAGG
ACTAGTAACAGAGGTCACAAAGTGGAGGTGAGCTGGGAACAGAGGGCTGCAGGGATGGGT
GGTGCTGGTCTGTAATAAGCTTTGAGAGCAACGTCACTGGGGCTTTGGGGTCAGCTACAC
AAGGAAGGCATTTGGACCCCTGCCTTTTCATTGCCCGAAACCAGAGCCTTTCCACCAAGC
GTTTCCCAGTCTTAGCCCTGTGTTCTGAGTTACGTACGATCTTTCTGGCAAATGGGGTGC
ATGATAAGAGCATCTCTTACGAAGAGTTGGAAAAACAAATGCCATATATAAATTCTAAGC
CATATGAGGACGAGGAGTAATGGCATTTTCTTCCTTTTTCCTCTCACTCCCAGACATTCA
TTGTCCCTGAATGCTCCATTAATCCAGGGAAGGTAATTGCCTAAATCTCCAGTGGATCTC
GCAACAGGAAGGAACCAGAAGCTGGGAAAGTTGTTTACCTCTTTGTCCCAGAGTTAGACC
TCATCCTCCCCTAGCTTAGCTGTCTCAGAGATATACTGGCCCTCCCTTCTCTTCTCTTTG
CTGCTGGTGCTAAAACTGCTCTGTAGGTCATTGGCCACTGTCTCCACTCACAACCCCTGC
TCCAGTCCTGGAGGGAGTGGGTTAAACACAAATAGAACATTCCATTTGAAGCAGTGATTC
TTTTTTTTTTTTTTTTTTTTTAATCAAATGCTTTGGACTTTTGAAGTCCACTTGTTCTGT
ACTTGTAAAAGGGAAAGAAGGCCGGGCGCAGTCGTCACGCCTGTAATCCCAGCACTTTAG
ATCACTTGAGGTCAGGAGTTTGAGACCAGCCCGGCCAACATGGTGAAACCCCATCTCTAC
TAAAAATACAAAAATTAGCTGTGCATAGTGGTTGGCACCTGTAGTCCCAGCTACTCAGGA
GGCTGAGGCAAGCTAACTGCTTGAACCCAGAAGGCAGAGGTTGCAGTGAGCTGAGATCAC
GCCACTGCACTCCAGCCTGGGTGACAGAGTGAGTGAGACTCTGCGTTAAAAAAAAAAAAA
AAAAAAAAAA
>gi|13186238|gb|NP_075595.1|FGFR1 302 aa linear fibroblast
growth factor receptor 1 isoform 5 precursor; fms-related
tyrosine kinase-2; heparin-binding growth factor receptor;
FMS-like tyrosine kinase 2; basic fibroblast growth factor
receptor 1; N-sam tyrosine kinase; FLG protein; protein-
tyrosine kinase; tyrosylprotein kinase; hydroxyaryl-protein
kinase [Homo sapiens].
MWSWKCLLFWAVLVTATLCTARPSPTLPEQDALPSSEDDDDDDDSSSEEKETDNTKPNRM
PVAPYWTSPEKMEKKLHAVPAAKTVKFKCPSSGTPNPTLRWLKNGKEFKPDHRIGGYKVR
YATWSIIMDSVVPSDKGNYTCIVENEYGSINHTYQLDVVERSPHRPILQAGLPANKTVAL
GSNVEFMCKVYSDPQPHIQWLKHIEVNGSKIGPDNLPYVQILKVIMAPVFVGQSTGKETT
VSGAQVPVGRLSCPRMGSFLTLQAHTLHLSRDLATSPRTSNRGHKVEVSWEQRAAGMGGA
GL
>gi|4758007|gb|NM_004071.1|CLK1 1834 bp mRNA Homo sapiens
CDC-like kinase 1 (CLK1), mRNA.
ATTTTTAGATAATCATTAAAGACCACAGAAAATGTAACAGATCCTACTCTTCAAAATAAT
TGCTATTCAGTATTAAAACGAGCAGTCAGCTGCGTGATTCCCGTGATTGCGTTACAAGCT
TTGTCTCCTTCGACTTGGAGTCTTTGTCCAGGACGATGAGACACTCAAAGAGAACTTACT
GTCCTGATTGGGATGACAAGGATTGGGATTATGGAAAATGGAGGAGCAGCAGCAGTCATA
AAAGAAGGAAGAGATCACATAGCAGTGCCCAGGAGAACAAGCGCTGCAAATACAATCACT
CTAAAATGTGTGATAGCCATTATTTGGAAAGCAGGTCTATAAATGAGAAAGATTATCATA
GTCGACGCTACATTGATGAGTACAGAAATGACTACACTCAAGGATGTGAACCTGGACATC
GCCAAAGAGACCATGAAAGCCGGTATCAGAACCATAGTAGCAAGTCTTCTGGTAGAAGTG
GAAGAAGTAGTTATAAAAGCAAACACAGGATTCACCACAGTACTTCACATCGTCGTTCAC
ATGGGAAGAGTCACCGAAGGAAAAGAACCAGGAGTGTAGAGGATGATGAGGAGGGTCACC
TGATCTGTCAGAGTGGAGACGTACTAAGTGCAAGATATGAAATTGTTGATACTTTAGGTG
AAGGAGCTTTTGGAAAAGTTGTGGAGTGCATCGATCATAAAGCGGGAGGTAGACATGTAG
CAGTAAAAATAGTTAAAAATGTGGATAGATACTGTGAAGCTGCTCGCTCAGAAATACAAG
TTCTGGAACATCTGAATACAACAGACCCCAACAGTACTTTCCGCTGTGTCCAGATGTTGG
AATGGTTTGAGCATCATGGTCACATTTGCATTGTTTTTGAACTATTGGGACTTAGTACTT
ACGACTTCATTAAAGAAAATGGTTTTCTACCATTTCGACTGGATCATATCAGAAAGATGG
CATATCAGATATGCAAGTCTGTGAATTTTTTGCACAGTAATAAGTTGACTCACACAGACT
TAAAGCCTGAAAACATCTTATTTGTGCAGTCTGACTACACAGAGGCGTATAATCCCAAAA
TAAAACGTGATGAACGCACCTTAATAAATCCAGATATTAAAGTTGTAGACTTTGGTAGTG
CAACATATGATGACGAACATCACAGTACATTGGTATCTACAAGACATTATAGAGCACCTG
AAGTTATTTTAGCCCTAGGGTGGTCCCAACCATGTGATGTCTGGAGCATAGGATGCATTC
TTATTGAATACTATCTTGGGTTTACCGTATTTCCAACACACGATAGTAAGGAGCATTTAG
CAATGATGGAAAGGATTCTTGGACCTCTACCAAAACATATGATACAGAAAACCAGGAAAC
GTAAATATTTTCACCACGATCGATTAGACTGGGATGAACACAGTTCTGCCGGCAGATATG
TTTCAAGAGCCTGTAAACCTCTGAAGGAATTTATGCTTTCTCAAGATGTTGAACATGAGC
GTCTCTTTGACCTCATTCAGAAAATGTTGGAGTATGATCCAGCCAAAAGAATTACTCTCA
GAGAAGCCTTAAAGCATCCTTTCTTTGACCTTCTGAAGAAAAGTATATAGATCTGTAATT
GGACAGCTCTCTCGAAGAGATCTTACAGACTGTATCAGTCTAATTTTTAAATTTTAAGTT
ATTTTGTACAGCTTTGTAAATTCTTAACATTTTTATATTGCCATGTTTATTTTGTTTGGG
TAATTTGGTTCATTAAGTACATAGCTAAGGTAATGAACATCTTTTTCAGTAATTGTAAAG
TGATTTATTCAGAATAAATTTTTTGTGCTTATGA
>gi|4758008|gb|NP_004062.1|CLK1 484 aa linear CDC-like
kinase 1; protein tyrosine kinase STY [Homo sapiens].
MRHSKRTYCPDWDDKDWDYGKWRSSSSHKRRKRSHSSAQENKRCKYNHSKMCDSHYLESR
SINEKDYHSRRYIDEYRNDYTQGCEPGHRQRDHESRYQNHSSKSSGRSGRSSYKSKHRIH
HSTSHRRSHGKSHRRKRTRSVEDDEEGHLICQSGDVLSARYEIVDTLGEGAFGKVVECID
HKAGGRHVAVKIVKNVDRYCEAARSEIQVLEHLNTTDPNSTFRCVQMLEWFEHHGHICIV
FELLGLSTYDFIKENGFLPFRLDHIRKMAYQICKSVNFLHSNKLTHTDLKPENILFVQSD
YTEAYNPKIKRDERTLINPDIKVVDFGSATYDDEHHSTLVSTRHYPAPEVILALGWSQPC
DVWSIGCILIEYYLGFTVFPTHDSKEHLAMMERILGPLPKHMIQKTRKRKYFHHDRLDWD
EHSSAGRYVSRACKPLKEFMLSQDVEHERLFDLIQKMLEYDPAKRITLREALKHPFFDLL
KKSI
>gi|20127640|gb|NM_025128.2|MUS81 2352 bp mRNA Homo sapiens
MUS81 endonuclease (MUS81), mRNA.
GGCACGAGGGTCTCAAAGGCTGGCTGGAGTGGAGCCAAAGGAAAAGATCGTTAGAGACAG
CGCCCCTGACCAACCACTTAGAGCAGCGCAGGGGTGGGAGGGCGGCCGCAGGCTCTCCTC
TCGTTAGTGCCCCCTGTGTTTGGGGCCCCGTGATCTCAACGGTCCTGCCCTCGGTCTCCC
TCTTCCCCCGCCCCGCCCTGGGCCAGGTGTTCGAATCCCGACTCCAGAACTGGCGGCGTC
CCAGTCCCGCGGGCGTGGAGCGCCGGAGGACCCGCCCTCGGGCTCATGGCGGCCCCGGTC
CGCCTGGGCCGGAAGCGCCCGCTGCCTGCCTGTCCCAACCCGCTCTTCGTTCGCTGGCTG
ACCGAGTGGCGGGACGAGGCGACCCGCAGCAGGCACCGCACGCGCTTCGTATTTCAGAAG
GCGCTGCGTTCCCTCCGACGGTACCCACTGCCGCTGCGCAGCGGGAAGGAAGCTAAGATC
CTACAGCACTTCGGAGACGGGCTCTGCCGGATGCTGGACGAGCGGCTGCAGCGGCACCGA
ACATCGGGCGGTGACCATGCCCCGGACTCACCATCTGGAGAGAACAGTCCAGCCCCGCAG
GGGCGACTTGCGGAAGTCCAGGACTCTTCCATGCCAGTTCCTGCCCAGCCCAAAGCGGGA
GGCTCTGGCAGCTACTGGCCAGCTCGGCACTCAGGAGCCCGAGTGATACTGCTGGTGCTC
TACCGGGAGCACCTGAATCCTAATGGTCACCACTTCTTAACCAAGGAGGAGCTGCTGCAG
AGGTGTGCTCAGAAGTCCCCCAGGGTAGCCCCTGGGAGTGCCCCACCCTGGCCAGCCCTC
CGCTCCCTCCTTCACAGGAACCTGGTCCTCAGGACACACCAGCCAGCCAGGTACTCATTG
ACCCCAGAGGGCCTGGAGCTGGCCCAGAAGTTGGCCGAGTCAGAAGGCCTGAGCTTGCTG
AATGTGGGCATCGGGCCCAAGGAGCCCCCTGGGGAGGAGACAGCAGTGCCAGGAGCAGCT
TCAGCAGAGCTTGCCAGTGAAGCAGGGGTCCAGCAGCAGCCACTGGAGCTGAGGCCTGGA
GAGTACAGGGTGCTGTTGTGTGTGGACATTGGCGAGACCCGGGGGGGCGGGCACAGGCCG
GAGCTGCTCCGAGAGCTACAGCGGCTGCACGTGACCCACACGGTGCGCAAGCTGCACGTT
GGAGATTTTGTGTGGGTGGCTCAGGAGACCAATCCTAGAGACCCAGCAAACCCTGGGGAG
TTGGTACTGGATCACATTGTGGAGCGCAAGCGACTGGATGACCTTTGCAGCAGCATCATC
GACGGCCGCTTCCGGGAGCAGAAGTTCCGACTGAAGCGCTGTGGTCTGGAGCGCCGGGTA
TACCTGGTGGAAGAGCATGGTTCCGTCCACAACCTCAGCCTTCCTGAGAGCACACTGCTG
CAGGCTGTCACCAAGACTCAGGTCATTGATGGCTTTTTTGTGAAGCGCACAGCAGACATT
AAGGAGTCAGCCGCCTACCTGGCCCTCTTGACTCGGGGCCTGCAGAGACTCTACCAGGGC
CACACCCTACGCAGCCGCCCCTGGGGAACCCCTGGGAACCCTGAATCAGGGGCCATGACC
TCTCCAAACCCTCTCTGCTCACTCCTCACCTTCAGTGACTTCAACGCAGGAGCCATCAAG
AATAAGGCCCAGTCGGTGCGAGAAGTGTTTGCCCGGCAGCTGATGCAGGTGCGCGGAGTG
AGTGGGGAGAAGGCAGCAGCCCTGGTGGATCGATACAGCACCCCTGCCAGCCTCCTGGCC
GCCTATGATGCCTGTGCCACCCCCAAGGAACAAGAGACACTGCTGAGCACCATTAAGTGT
GGGCGTCTACAGAGGAATCTGGGGCCTGCTCTGAGCAGGACCTTATCCCAGCTCTACTGC
AGCTACGGCCCCTTGACCTGAGCTTATGCCGTGAAACAGCCCCCAGCCCCCGTCTGTCCC
CCAACCCAGGCTAGCCAGCCTTTTAACAACATCTTTTGGGGTACAATTAGAATCTAAGTG
TTTGCAGCCATATGTGTCATGTAGAAGATGCCTAGCCCTGGGGACCTTGTGAAATACGCA
GGAACCAGGGATACCATCTGGTCCAGTGGTTTTTAAACAAAGCTGCTTAGCACCTGGAAT
TCCCTGGTCAGGGAGATGGAGTCAGTGGGGCATTGCAGCTTGGAATCTATTTTATGTCAC
CAGTTGGTCCTCATCAAATAAAATTTCCTTAGGAGTGCAGAGGGCTCATTGGGAAAATAA
AAATAATAAAAATAAATAAAACTTCCTAAAAGAAAAGATTGAAACCCAAAAAAAAAAAAA
AAAAAAAAAAAA
>gi|13376707|gb|NP_079404.1|MUS82 476 aa linear MUS81
endonuclease [Homo sapiens].
MLDERLQRHRTSGGDHAPDSPSGENSPAPQGRLAEVQDSSMPVPAQPKAGGSGSYWPARH
SGARVILLVLYREHLNPNGHHFLTKEELLQRCAQKSPRVAPGSAPPWPALRSLLHRNLVL
RTHQPARYSLTPEGLELAQKLAESEGLSLLNVGIGPKEPPGEETAVPGAASAELASEAGV
QQQPLELRPGEYRVLLCVDIGETRGGGHRPELLRELQRLHVTHTVRKLHVGDFVWVAQET
NPRDPANPGELVLDHIVERKRLDDLCSSIIDGRFREQKFRLKRCGLERRVYLVEEHGSVH
NLSLPESTLLQAVTNTQVIDGFFVKRTADIKESAAYLALLTRGLQRLYQGHTLRSRPWGT
PGNPESGAMTSPNPLCSLLTFSDFNAGAIKNKAQSVREVFARQLMQVRGVSGEKAAALVD
RYSTPASLLAAYDACATPKEQETLLSTIKCGRLQRNLGPALSRTLSQLYCSYGPLT
>gi|19923239|gb|NM_003376.2|VEGF 3166 bp mRNA Homo sapiens
vascular endothelial growth factor (VEGF), mRNA.
AAGAGCTCCAGAGAGAAGTCGAGGAAGAGAGAGACGGGGTCAGAGAGAGCGCGCGGGCGT
GCGAGCAGCGAAAGCGACAGGGGCAAAGTGAGTGACCTGCTTTTGGGGGTGACCGCCGGA
GCGCGGCGTGAGCCCTCCCCCTTGGGATCCCGCAGCTGACCAGTCGCGCTGACGGACAGA
CAGACAGACACCGCCCCCAGCCCCAGTTACCACCTCCTCCCCGGCCGGCGGCGGACAGTG
GACGCGGCGGCGAGCCGCGGGCAGGGGCCGGAGCCCGCCCCCGGAGGCGGGGTGGAGGGG
GTCGGAGCTCGCGGCGTCGCACTGAAACTTTTCGTCCAACTTCTGGGCTGTTCTCGCTTC
GGAGGAGCCGTGGTCCGCGCGGGGGAAGCCGAGCCGAGCGGAGCCGCGAGAAGTGCTAGC
TCGGGCCGGGAGGAGCCGCAGCCGGAGGAGGGGGAGGAGGAAGAAGAGAAGGAAGAGGAG
AGGGGGCCGCAGTGGCGACTCGGCGCTCGGAAGCCGGGCTCATGGACGGGTGAGGCGGCG
GTGTGCGCAGACAGTGCTCCAGCGCGCGCGCTCCCCAGCCCTGGCCCGGCCTCGGGCCGG
GAGGAAGAGTAGCTCGCCGAGGCGCCGAGGAGAGCGGGCCGCCCCACAGCCCGAGCCGGA
GAGGGACGCGAGCCGCGCGCCCCGGTCGGGCCTCCGAAACCATGAACTTTCTGCTGTCTT
GGGTGCATTGGAGCCTTGCCTTGCTGCTCTACCTCCACCATGCCAAGTGGTCCCAGGCTG
CACCCATGGCAGAAGGAGGAGGGCAGAATCATCACGAAGTGGTGAAGTTCATGGATGTCT
ATCAGCGCAGCTACTGCCATCCAATCGAGACCCTGGTGGACATCTTCCAGGAGTACCCTG
ATGAGATCGAGTACATCTTCAAGCCATCCTGTGTGCCCCTGATGCGATGCGGGGGCTGCT
CCAATGACGAGGGCCTGGAGTGTGTGCCCACTGAGGAGTCCAACATCACCATGCAGATTA
TGCGGATCAAACCTCACCAAGGCCAGCACATAGGAGAGATGAGCTTCCTACAGCACAACA
AATGTGAATGCAGACCAAAGAAAGATAGAGCAAGACAAGAAAATCCCTGTGGGCCTTGCT
CAGAGCGGAGAAAGCATTTGTTTGTACAAGATCCGCAGACGTGTAAATGTTCCTGCAAAA
ACACACACTCGCGTTGCAAGGCGAGGCAGCTTGAGTTAAACGAACGTACTTGCAGATGTG
ACAAGCCGAGGCGGTGAGCCGGGCAGGAGGAAGGAGCCTCCCTCAGGGTTTCGGGAACCA
GATCTCTCTCCAGGAAAGACTGATACAGAACGATCGATACAGAAACCACGCTGCCGCCAC
CACACCATCACCATCGACAGAACAGTCCTTAATCCAGAAACCTGAAATGAAGGAAGAGGA
GACTCTGCGCAGAGCACTTTGGGTCCGGAGGGCGAGACTCCGGCGGAAGCATTCCCGGGC
GGGTGACCCAGCACGGTCCCTCTTGGAATTGGATTCGCCATTTTATTTTTCTTGCTGCTA
AATCACCGAGCCCGGAAGATTAGAGAGTTTTATTTCTGGGATTCCTGTAGACACACCCAC
CCACATACATACATTTATATATATATATATTATATATATATAAAAATAAATATCTCTATT
TTATATATATAAAATATATATATTCTTTTTTTAAATTAACAGTGCTAATGTTATTGGTGT
CTTCACTGGATGTATTTGACTGCTGTGGACTTGAGTTGGGAGGGGAATGTTCCCACTCAG
ATCCTGACAGGGAAGAGGAGGAGATGAGAGACTCTGGCATGATCTTTTTTTTGTCCCACT
TGGTGGGGCCAGGGTCCTCTCCCCTGCCCAAGAATGTGCAAGGCCAGGGCATGGGGGCAA
ATATGACCCAGTTTTGGGAACACCGACAAACCCAGCCCTGGCGCTGAGCCTCTCTACCCC
AGGTCAGACGGACAGAAAGACAAATCACAGGTTCCGGGATGAGGACACCGGCTCTGACCA
GGAGTTTGGGGAGCTTCAGGACATTGCTGTGCTTTGGGGATTCCCTCCACATGCTGCACG
CGCATCTCGCCCCCAGGGGCACTGCCTGGAAGATTCAGGAGCCTGGGCGGCCTTCGCTTA
CTCTCACCTGCTTCTGAGTTGCCCAGGAGGCCACTGGCAGATGTCCCGGCGAAGAGAAGA
GACACATTGTTGGAAGAAGCAGCCCATGACAGCGCCCCTTCCTGGGACTCGCCCTCATCC
TCTTCCTGCTCCCCTTCCTGGGGTGCAGCCTAAAAGGACCTATGTCCTCACACCATTGAA
ACCACTAGTTCTGTCCCCCCAGGAAACCTGGTTGTGTGTGTGTGAGTGGTTGACCTTCCT
CCATCCCCTGGTCCTTCCCTTCCCTTCCCGAGGCACAGAGAGACAGGGCAGGATCCACGT
GCCCATTGTGGAGGCAGAGAAAAGAGAAAGTGTTTTATATACGGTACTTATTTAATATCC
CTTTTTAATTAGAAATTAGAACAGTTAATTTAATTAAAGAGTAGGGTTTTTTTTCAGTAT
TCTTGGTTAATATTTAATTTCAACTATTTATGAGATGTATCTTTTGCTCTCTCTTGCTCT
CTTATTTGTACCGGTTTTTGTATATAAAATTCATGTTTCCAATCTCTCTCTCCCTGATCG
GTGACAGTCACTAGCTTATCTTGAACAGATATTTAATTTTGCTAACACTCAGCTCTGCCC
TCCCCGATCCCCTGGCTCCCCAGCACACATTCCTTTGAAAGAGGGTTTCAATATACATCT
ACATACTATATATATATTGGGCAACTTGTATTTGTGTGTATATATATATATATATGTTTA
TGTATATATGTGATCCTGAAAAAATAAACATCGCTATTCTGTTTTTTATATGTTCAAACC
AAACAAGAAAAAATAGAGAATTCTACATACTAAATCTCTCTCCTTTTTTAATTTTAATAT
TTGTTATCATTTATTTATTGGTGCTACTGTTTATCCGTAATAATTGTGGGGAAAAGATAT
TAACATCACGTCTTTGTCTCTAGTGCAGTTTTTCGAGATATTCCGTAGTACATATTTATT
TTTAAACAACGACAAAGAAATACAGATATATCTTAAAAAAAAAAAA
>gi|19923240|gb|NP_003367.2|VEGF 191 aa linear vascular
endothelial growth factor [Homo sapiens].
MNFLLSWVHWSLALLLYLHHAKWSQAAPMAEGGGQNHHEVVKFMDVYQRSYCHPIETLVD
IFQEYPDEIEYIFKPSCVPLMRCGGCSNDEGLECVPTEESNITMQIMRIKPHQGQEIGEM
SFLQHNKCECRPKKDRARQENPCGPCSERRKHLFVQDPQTCKCSCKNTHSRCKARQLELN
ERTCRCDKPRR
>gi|16306545|gb|NM_033649.1|FGF18 1466 bp mRNA Homo sapiens
fibroblast growth factor 18 (FGF18), transcript variant 2,
mRNA.
CACGGCCGGAGAGACGCGGAGGAGGAGACATGAGCCGGCGGGCGCCCAGACGGAGCGGCC
GTGACGCTTTCGCGCTGCAGCCGCGCGCCCCGACCCCGGAGCGCTGACCCCTGGCCCCAC
GCAGCTCCGCGCCCGGGCCGGAGAGCGCAACTCGGCTTCCAGACCCGCCGCGCATGCTGT
CCCCGGACTGAGCCGGGCAGCCAGCCTCCCACGGACGCCCGGACGGCCGGCCGGCCAGCA
GTGAGCGAGCTTCCCCGCACCGGCCAGGCGCCTCCTGCACAGCGGCTGCCGCCCCGCAGC
CCCTGCGCCAGCCCGGAGGGCGCAGCGCTCGGGAGGAGCCGCGCGGGGCGCTGATGCCGC
AGGGCGCGCCGCGGAGCGCCCCGGAGCAGCAGAGTCTGCAGCAGCAGCAGCCGGCGAGGA
GGGAGCAGCAGCAGCGGCGGCGGCGGCGGCGGCGGCGGCGGAGGCGCCCGGTCCCGGCCG
CGCGGAGCGGACATGTGCAGGCTGGGCTAGGAGCCGCCGCCTCCCTCCCGCCCAGCGATG
TATTCACCGCCCTCCGCCTGCACTTGCCTGTGTTTACACTTCCTGCTGCTGTGCTTCCAG
GTACAGGTGCTGGTTGCCGAGGAGAACGTGGACTTCCGCATCCACGTGGAGAACCAGACG
CGGGCTCGGGACGATGTGAGCCGTAAGCAGCTGCGGCTGTACCAGCTCTACAGCCGGACC
AGTGGGAAACACATCCAGGTCCTGGGCCGCAGGATCAGTGCCCGCGGCGAGGATGGGGAC
AAGTATGCCCAGCTCCTAGTGGAGACAGACACCTTCGGTAGTCAAGTCCGGATCAAGGGC
AAGGAGACGGAATTCTACCTGTGCATGAACCGCAAAGGCAAGCTCGTGGGGAAGCCCGAT
GGCACCAGCAAGGAGTGTGTGTTCATCGAGAAGGTTCTGGAGAACAACTACACGGCCCTG
ATGTCGGCTAAGTACTCCGGCTGGTACGTGGGCTTCACCAAGAAGGGGCGGCCGCGGAAG
GGCCCCAAGACCCGGGAGAACCAGCAGGACGTGCATTTCATGAAGCGCTACCCCAAGGGG
CAGCCGGAGCTTCAGAAGCCCTTCAAGTACACGACGGTGACCAAGAGGTCCCGTCGGATC
CGGCCCACACACCCTGCCTAGGCCACCCCGCCGCGGCCCTCAGGTCGCCCTGGCCACACT
CACACTCCCAGAAAACTGCATCAGAGGAATATTTTTACATGAAAAATAAGGATTTTATTG
TTGACTTGAAACCCCCGATGACAAAAGACTCACGCAAAGGGACTGTAGTCAACCCACAGG
TGCTTGTCTCTCTCTAGGAACAGACAACTCTAAACTCGTCCCCAGAGGAGGACTTGAATG
AGGAAACCAACACTTTGAGAAACCAAAGTCCTTTTTCCCAAAGGTTCTGAAAGGAAAAAA
AAAAAAAAACAAAAAAAAAAAAAAAA
>gi|16306546|gb|NP_387498.1|FGF18 207 aa linear fibroblast
growth factor 18 precursor [Homo sapiens].
MYSAPSACTCLCLHFLLLCFQVQVLVAEENVDFRIHVENQTRARDDVSRKQLRLYQLYSR
TSGKHIQVLGRRISARGEDGDKYAQLLVETDTFGSQVRIKGKETEFYLCMNRKGKLVGKP
DGTSKECVFIEKVLENNYTALMSAKYSGWYVGFTKKGRPRKGPKTRENQQDVHFMKRYPK
GQPELQKPFKYTTVTKRSRRIRPTHPA
>gi|24496766|gb|NM_004712.3|HGS 2926 bp mRNA Homo sapiens
hepatocyte growth factor-regulated tyrosine kinase substrate
(HGS), mRNA.
CGGAAGCGGAAGTCGGGGGGCGCGCCAGCTCGTAGCAGGGGAGCGCCCGCGGCGTCGGGT
TTGGGCTGGAGGTCGCCATGGGGCGAGGCAGCGGCACCTTCGAGCGTCTCCTAGACAAGG
CGACCAGCCAGCTCCTGTTGGAGACAGATTGGGAGTCCATTTTGCAGATCTGCGACCTGA
TCCGCCAAGGGGACACACAAGCAAAATATGCTGTGAATTCCATCAAGAAGAAAGTCAACG
ACAAGAACCCACACGTCGCCTTGTATGCCCTGGAGGTCATGGAATCTGTGGTAAAGAACT
GTGGCCAGACAGTTCATGATGAGGTGGCCAACAAGCAGACCATGGAGGAGCTGAAGGACC
TGCTGAAGAGACAAGTGGAGGTAAACGTCCGTAACAAGATCCTGTACCTGATCCAGGCCT
GGGCGCATGCCTTCCGGAACGAGCCCAAGTACAAGGTGGTCCAGGACACCTACCAGATCA
TGAAGGTGGAGGGGCACGTCTTTCCAGAATTCAAAGAGAGCGATGCCATGTTTGCTGCCG
AGAGAGCCCCAGACTGGGTGGACGCTGAGGAATGCCACCGCTGCAGGGTGCAGTTCGGGG
TGATGACCCGTAAGCACCACTGCCGGGCGTGTGGGCAGATATTCTGTGGAAAGTGTTCTT
CCAAGTACTCCACCATCCCCAAGTTTGGCATCGAGAAGGAGGTGCGCGTGTGTGAGCCCT
GCTACGAGCAGCTGAACAGGAAAGCGGAGGGAAAGGCCACTTCCACCACTGAGCTGCCCC
CCGAGTACCTGACCAGCCCCCTGTCTCAGCAGTCCCAGCTGCCCCCCAAGAGGGACGAGA
CGGCCCTGCAGGAGGAGGAGGAGCTGCAGCTGGCCCTGGCGCTGTCACAGTCAGAGGCGG
AGGAGAAGGAGAGGCTGAGACAGAAGTCCACGTACACTTCGTACCCCAAGGCGGAGCCCA
TGCCCTCGGCCTCCTCAGCGCCCCCCGCCAGCAGCCTGTACTCTTCACCTGTGAACTCGT
CGGCGCCTCTGGCTGAGGACATCGACCCTGAGCTCGCACGGTATCTCAACCGGAACTACT
GGGAGAAGAAGCAGGAGGAGGCTCGCAAGAGCCCCACGCCATCTGCGCCCGTGCCCCTGA
CGGAGCCGGCTGCACAGCCTGGGGAAGGGCACGCAGCCCCCACCAACGTGGTGGAGAACC
CCCTCCCGGAGACAGACTCTCAGCCCATTCCTCCCTCTGGTGGCCCCTTTAGTGAGCCAC
AGTTCCACAATGGCGAGTCTGAGGAGAGCCACGAGCAGTTCCTGAAGGCGCTGCAGAACG
CCGTCACCACCTTCGTGAACCGCATGAAGAGTAACCACATGCGGGGCCGCAGCATCACCA
ATGACTCGGCCGTGCTCTCACTCTTCCAGTCCATCAACGGCATGCACCCGCAGCTGCTGG
AGCTGCTCAACCAGCTGGACGAGCGCAGGCTGTACTATGAGGGGCTGCAGGACAAGCTGG
CACAGATCCGCGATGCCCGGGGGGCGCTGAGTGCCCTGCGCGAAGAGCACCGGGAGAAGC
TTCGCCGGGCAGCCGAGGAGGCAGAGCGCCAGCGCCAGATCCAGCTGGCCCAGAAGCTGG
AGATAATGCGGCAGAAGAAGCAGGAGTACCTGGAGGTGCAGAGGCAGCTGGCCATCCAGC
GCCTGCAGGAGCAGGAGAAGGAGCGGCAGATGCGGCTGGAGCAGCAGAAGCAGACGGTCC
AGATGCGCGCGCAGATGCCCGCCTTCCCCCTGCCCTACGCCCAGCTCCAGGCCATGCCCG
CAGCCGGAGGTGTGCTCTACCAGCCCTCGGGACCAGCCAGCTTCCCCAGCACCTTCAGCC
CTGCCGGCTCGGTGGAGGGCTCCCCAATGCACGGCGTGTACATGAGCCAGCCGGCCCCTG
CCGCTGGCCCCTACCCCAGCATGCCCAGCACTGCGGCTGATCCCAGCATGGTGAGTGCCT
ACATGTACCCAGCAGGGGCCACTGGGGCGCAGGCGGCCCCCCAGGCCCAGGCCGGACCCA
CCGCCAGCCCCGCTTACTCATCCTACCAGCCTACTCCCACAGCGGGCTACCAGAACGTGG
CCTCCCAGGCCCCACAGAGCCTCCCGGCCATCTCTCAGCCTCCGCAGTCCAGCACCATGG
GCTACATGGGGAGCCAGTCAGTCTCCATGGGCTACCAGCCTTACAACATGCAGAATCTCA
TGACCACCCTCCCAAGCCAGGATGCGTCTCTGCCACCCCAGCAGCCCTACATCGCGGGGC
AGCAGCCCATGTACCAGCAGATGGCACCCTCTGGCGGTCCCCCCCAGCAGCAGCCCCCCG
TGGCCCAGCAACCGCAGGCACAGGGGCCGCCGGCACAGGGCAGCGAGGCCCAGCTCATTT
CATTCGACTGACCCAGGCCATGCTCACGTCCGGAGTAACACTACATACAGTTCACCTGAA
ACGCCTCGTCTCTAACTGCCGTCGTCCTGCCTCCCTGTCCTCTACTGCCGGTAGTGTCCC
TTCTCTGCGAGTGAGGGGGGGCCTTCACCCCAAGCCCACCTCCCTTGTCCTCAGCCTACT
GCAGTCCCTGAGTTAGTCTCTGCTTTCTTTCCCCAGGGCTGGGCCATGGGGAGGGAAGGA
CTTTCTCCCAGGGGAAGCCCCCAGCCCTGTGGGTCATGGTCTGTGAGAGGTGGCAGGAAT
GGGGACCCTCACCCCCCAAGCAGCCTGTGCCCTCTGGCCGCACTGTGAGCTGGCTGTGGT
GTCTGGGTGTGGCCTGGGGCTCCCTCTGCAGGGGCCTCTCTCGGCAGCCACAGCCAAGGG
TGGAGGCTTCAGGTCTCCAGCTTCTCTGCTTCTCAGCTGCCATCTCCAGTGCCCCAGAAT
GGTACAGCGATAATAAAATGTATTTCAGAAAAAAAAAAAAAAAAAA
>gi|4758528|gb|NP_004703.1|HGS 777 aa linear hepatocyte
growth factor-regulated tyrosine kinase substrate; human
growth factor-regulated tyrosine kinase substrate [Homo
sapiens].
MGRGSGTFERLLDKATSQLLLETDWESILQICDLIRQGDTQAKYAVNSIKKKVNDKNPHV
ALYALEVMESVVKNCGQTVHDEVANKQTMEELKDLLKRQVEVNVRNKILYLIQAWAHAFR
NEPKYKVVQDTYQIMKVEGHVFPEFKESDAMFAAERAPDWVDAEECHRCRVQFGVMTRKH
HCRACGQIFCGKCSSKYSTIPKFGIEKEVRVCEPCYEQLNRKAEGKATSTTELPPEYLTS
PLSQQSQLPPKRDETALQEEEELQLALALSQSEAEEKERLRQKSTYTSYPKAEPMPSASS
APPASSLYSSPVNSSAPLAEDIDPELARYLNRNYWEKKQEEARKSPTPSAPVPLTEPAAQ
PGEGHAAPTNVVENPLPETDSQPIPPSGGPFSEPQFHNGESEESHEQFLKALQNAVTTFV
NRMKSNHMRGRSITNDSAVLSLFQSINGMHPQLLELLNQLDERRLYYEGLQDKLAQIRDA
RGALSALREEHREKLRRAAEEAERQRQIQLAQKLEIMRQKKQEYLEVQRQLAIQRLQEQE
KERQMRLEQQKQTVQMRAQMPAFPLPYAQLQAMPAAGGVLYQPSGPASFPSTFSPAGSVE
GSPMHGVYMSQPAPAAGPYPSMPSTAADPSMVSAYMYPAGATGAQAAPQAQAGPTASPAY
SSYQPTPTAGYQNVASQAPQSLPAISQPPQSSTMGYMGSQSVSMGYQPYNMQNLMTTLPS
QDASLPPQQPYIAGQQPMYQQMAPSGGPPQQQPPVAQQPQAQGPPAQGSEAQLISFD
>gi|20127435|gb|NM_003821.2|RIPK2 1898 bp mRNA Homo sapiens
receptor-interacting serine-threonine kinase 2 (RIPK2),
mRNA.
GGCACGAGGGTCAGCTCTGGTTCGGAGAAGCAGCGGCTGGCGTGGGCCATCCGGGGAATG
GGCGCCCTCGTGACCTAGTGTTGCGGGGCAAAAAGGGTCTTGCCGGCCTCGCTCGTGCAG
GGGCGTATCTGGGCGCCTGAGCGCGGCGTGGGAGCCTTGGGAGCCGCCGCAGCAGGGGGC
ACACCCGGAACCGGCCTGAGCGCCCGGGACCATGAACGGGGAGGCCATCTGCAGCGCCCT
GCCCACCATTCCCTACCACAAACTCGCCGACCTGCGCTACCTGAGCCGCGGCGCCTCTGG
CACTGTGTCGTCCGCCCGCCACGCAGACTGGCGCGTCCAGGTGGCCGTGAAGCACCTGCA
CATCCACACTCCGCTGCTCGACAGTGAAAGAAAGGATGTCTTAAGAGAAGCTGAAATTTT
ACACAAAGCTAGATTTAGTTACATTCTTCCAATTTTGGGAATTTGCAATGAGCCTGAATT
TTTGGGAATAGTTACTGAATACATGCCAAATGGATCATTAAATGAACTCCTACATAGGAA
AACTGAATATCCTGATGTTGCTTGGCCATTGAGATTTCGCATCCTGCATGAAATTGCCCT
TGGTGTAAATTACCTGCACAATATGACTCCTCCTTTACTTCATCATGACTTGAAGACTCA
GAATATCTTATTGGACAATGAATTTCATGTTAAGATTGCAGATTTTGGTTTATCAAAGTG
GCGCATGATGTCCCTCTCACAGTCACGAAGTAGCAAATCTGCACCAGAAGGAGGGACAAT
TATCTATATGCCACCTGAAAACTATGAACCTGGACAAAAATCAAGGGCCAGTATCAAGCA
CGATATATATAGCTATGCAGTTATCACATGGGAAGTGTTATCCAGAAAACAGCCTTTTGA
AGATGTCACCAATCCTTTGCAGATAATGTATAGTGTGTCACAAGGACATCGACCTGTTAT
TAATGAAGAAAGTTTGCCATATGATATACCTCACCGAGCACGTATGATCTCTCTAATAGA
AAGTGGATGGGCACAAAATCCAGATGAAAGACCATCTTTCTTAAAATGTTTAATAGAACT
TGAACCAGTTTTGAGAACATTTGAAGAGATAACTTTTCTTGAAGCTGTTATTCAGCTAAA
GAAAACAAAGTTACAGAGTGTTTCAAGTGCCATTCACCTATGTGACAAGAAGAAAATGGA
ATTATCTCTGAACATACCTGTAAATCATGGTCCACAAGAGGAATCATGTGGATCCTCTCA
GCTCCATGAAAATAGTGGTTCTCCTGAAACTTCAAGGTCCCTGCCAGCTCCTCAAGACAA
TGATTTTTTATCTAGAAAAGCTCAAGACTGTTATTTTATGAAGCTGCATCACTGTCCTGG
AAATCACAGTTGGGATAGCACCATTTCTGGATCTCAAAGGGCTGCATTCTGTGATCACAA
GACCACTCCATGCTCTTCAGCAATAATAAATCCACTCTCAACTGCAGGAAACTCAGAACG
TCTGCAGCCTGGTATAGCCCAGCAGTGGATCCAGAGCAAAAGGGAAGACATTGTGAACCA
AATGACAGAAGCCTGCCTTAACCAGTCGCTAGATGCCCTTCTGTCCAGGGACTTGATCAT
GAAAGAGGACTATGAACTTGTTAGTACCAAGCCTACAAGGACCTCAAAAGTCAGACAATT
ACTAGACACTACTGACATCCAAGGAGAAGAATTTGCCAAAGTTATAGTACAAAAATTGAA
AGATAACAAACAAATGGGTCTTCAGCCTTACCCGGAAATACTTGTGGTTTCTAGATCACC
ATCTTTAAATTTACTTCAAAATAAAAGCATGTAAGTGACTGTTTTTCAAGAAGAAATGTG
TTTCATAAAAGGATATTTATAAAAAAAAAAAAAAAAAA
>gi|4506537|gb|NP_003812.1|RIPK2 540 aa linear receptor-
interacting serine-threonine kinase 2; receptor interacting
protein 2 [Homo sapiens].
MNGEAICSALPTIPYHKLADLRYLSRGASGTVSSARRADWRVQVAVKHLHIHTPLLDSER
KDVLREAEILHKARFSYILPILGICNEPEFLGIVTEYMPNGSLNELLHRKTEYPDVAWPL
RFRILHEIALGVNYLHNMTPPLLHHDLKTQNILLDNEFHVKIADFGLSKWRMMSLSQSRS
SKSAPEGGTIIYMPPENYEPGQKSRASIKHDIYSYAVITWEVLSRKQPFEDVTNPLQIMY
SVSQGHRPVINEESLPYDIPHRARMISLIESGWAQNPDERPSFLKCLIELEPVLRTFEEI
TFLEAVIQLKKTKLQSVSSAIHLCDKKKMELSLNIPVNHGPQEESCGSSQLHENSGSPET
SRSLPAPQDNDFLSRKAQDCYFMKLHHCPGNHSWDSTISGSQRAAFCDHKTTPCSSAIIN
PLSTAGNSERLQPGIAQQWIQSKREDIVNQMTEACLNQSLDALLSRDLIMKEDYELVSTK
PTRTSKVRQLLDTTDIQGEEFAKVIVQKLKDNKQMGLQPYPEILVVSRSPSLNLLQNKSM
>gi|26051238|gb|NM_021137.3|TNFAIP1 3571 bp mRNA Homo
sapiens tumor necrosis factor, alpha-induced protein 1
(endothelial) (TNFAIP1), mRNA.
CACAGCTTGGGACTGCTGAGGGGCAGGCGGCTGCAGGCTAGGGGCGGCTCGGAGTCCGCT
GGCCACCCAGCTGAGAGGAGAGGCGCCCCCGGGGACGCACTGAGATTATGAGGCTCTGGC
CTCCACTGGCCACTCACTCGTGACCCTTTCCACCACGGCGGAGCCTTCCAAGCCTACCTC
CTGCCGTGTGGTGATCTACCTGCAGCGGGAGATGTCGGGGGACACCTGCCTGTGCCCAGC
CTCAGGGGCCAAGCCCAAGCTCAGTGGCTTCAAGGGAGGAGGGTTGGGCAACAAGTATGT
CCAGCTCAACGTGGGCGGCTCTCTGTACTACACCACTGTGCGGGCCCTGACCCGCCACGA
CACCATGCTCAAGGCCATGTTCAGTGGGCGCATGGAGGTGCTGACCGACAAAGAAGGCTG
GATCCTCATAGACCGTTGTGGAAAGCACTTTGGCACCATTTTGAATTACCTCCGAGATGA
CACCATCACCCTCCCTCAGAACCGGCAAGAAATCAAGGAATTGATGGCTGAAGCAAAGTA
TTACCTCATCCAGGGGCTGGTGAATATGTGCCAGAGTGCCCTGCAGGACAAGAAGGACTC
CTACCAGCCTGTGTGCAACATCCCCATCATCACATCCCTAAAGGAGGAGGAGCGGCTCAT
CGAATCCTCCACCAAGCCCGTGGTGAAGCTGCTGTACAACAGAAGCAACAACAAGTATTC
CTACACCAGCAACTCTGACGACCACCTGCTGAAAAACATCGAGCTGTTTGACAAGCTCTC
CCTGCGCTTCAACGGCCGCGTGCTCTTCATCAAGGATGTCATTGGTGACGAGATCTGCTG
CTGGTCCTTTTATGGCCAGGGCCGTAAGCTGGCAGAGGTGTGCTGTACCTCCATCGTGTA
TGCCACGGAGAAGAAGCAGACCAAGGTGGAATTCCCAGAGGCCCGAATCTATGAGGAGAC
ACTCAACGTCCTACTCTATGAGACTCCCCGCGTCCCCGACAACTCCTTGTTGGAGGCCAC
AAGCCGTAGCCGCAGCCAGGCTTCCCCCAGTGAAGATGAGGAGACCTTTGAACTGCGGGA
CCGTGTCCGCCGCATCCACGTCAAGCGCTACAGCACTTACGATGACCGGCAGCTCGGCCA
CCAGTCTACCCATCGCGACTGACCAGACCCTCAGGGAGTCAGGGCACGGGAGGCCCTATC
TCCCATCCTGTGGAACCCGCCCCATTGGCCACCCCATGCTGCTGCTGCCTGGGTCTCTGC
TCTAGCACCCAGAGGCATGACAGGCCCTGCTCAGAGGTCAGAGGGTCTGGGCAGAGGAGG
GACCACATTCCCCTGCCTTGCCCCTGAGCACTTCTGGAGACTGCGTCCTGTCCTATCTGC
TCACCATCACCCTTCCTGCCCGACGGAGCTGCTTCTGCTCCCTGGGGCATATGGACTGAC
CCACCTCCTGCTGAGAACCTTCCCCTAGGCCCTGTGCAGAAGGGCTACTGCCCCTTAGGC
CTCAGCTGGGGGAAAGGCAGTTCTGGTGCTGTAGAGGCCCTGGTGCAGAAAGTGGGACGT
CTTTTTTCCTAAGGTGTTTAAGCACAGGCTTGATAAGTTTGGTTTTTAAAAAATAATCTA
GGAAATGAATAATTCTAAATCTAGTAATGAGGAAACTGAGCATTTCTTTTGCCCTCCAGG
GTGCCAAGACCCTACATATGACAGAACCCTTGGCCCTTCTCCATGCCTGTGGGATCTGTT
TCTTTAAAGCACTTTGTACTGTTATTCAGGAGGTTGATAATCTCCTTGACCCATGTCTTT
CTACCCTAATCCCCACTTCCCTGCAGAATCAATCTGAGGGAGGGGATAAAGAGGAAGCAA
TAAAAAAAAAACATCCGACAGAGCAGCTCTGGCTTTGCCAGCCTGGCCAGCAGCTCAGAG
TGCACCGAGGAGGGAAGGATGGCTAAGCTGGGACCGGCAGTCCTCACAGGGTGCCTGTGA
GAAAGGACATTTTACCCCCACATCATAGTCACATCACTGACTCCTAGGTCTAGCACGACT
GCTCTTTGTGATTCTCTTGAGTACCCTTGGCTTCCAGCCATGCTGTCCTCACATACGGTA
AAGCCAAAGAGCTGTCACATGGGCCAGAAACATGAGCCACGGCAGGAAGACCGTGGAGCC
CGTGGGCACTGCATGGTGTTGGCTGGCATGCCCATCAGCTGAGGACAGCAAACTCCCAGC
AGCCCCTACAGAGGTGGCACATGCTTGGCCACACATCTACTCCCTGCCCACACCATCTAT
GCTCTTGGTTGGTGCTGGCTGGGATGGCGGTTCTGCCCAGTGGTGTCTCTGAGCGCGGGA
TGACAGGAGCAACCGAAGCACCCTGAAGGCCTTCACTCCTTGTTGGGTAACTCAGCCATG
GAGATGCCAAGCACTAGCCAGGAGGTGAGTTCCTCTTTAGGGCTTTGGTTTTCATTCCTT
TTTGTTTGGCTTGGCCAAACCAGAATTCAGCTTATCTGAATTATTTTCCAAAGGAATGCT
GTCAGGGAGGGACTGTTCTGCCAGCCTAACAAAGCAACGTAGCCACGTATAGTACCCACT
TTCTGCTCTTTGGAGAGAACACAGGTTATCAAGTTCATCTCTCTTGACTACTCTTATGAT
AGCTGATGCCACAGAGCCTATGGGCAAATGCCAGACCCAGGGTTAGACACAAGGACCTGA
AGTGACATGACGGCGGGACAGGGGAAATGTGACTTTCTAATTAGGCATTTTATGTTAGTC
ACAGTCTTGAATGTATAAACAGCACTAAGACTCTCAGGTCAGGTACCTTGGTGATCAGCT
ACTAGTTCTTCCAGCCCTCATTGAGGTAACAAGATAAAGACAAATCCACTTCTTTGGCCA
AATTCAGGCTTTGGCTTTATGACTTTCCCACAGAGACTGGAATGCGTCAGCCTGAGACCA
CTGGCCTATTTTCTCAGCTGCCCTCTTGAGGTCCTTTAACACTCAAATTCCCAGCTCCCC
ACTGAGGTGTTGTGATGCTTGCCTTTTGACCTCCCCATCCCCTTTAGTCCCTGCTTACTA
CTTTGACATTCACATCCTCAGTGTCTCAGTCTTTTTTGCCGAGAAAGCACAGTAGTCTGG
GACTGGGCATTTATCTTCTCTGACTGAAAATCTCTCCTTGGTCTTAAGGAAAATACTAAC
ATTGAACTCACTGACATGATCTTAGCTTCTTTAATCAGACTTTGTGACTTAAAAGTTTGG
GGGTTTTCTTTGAAAGTTTCCAGCCCTATTCAGAAAGCAACTCTTGGCTGTGTGCATTTT
TCAACTCCAAGCAGCCCAGGGGTAAGTAAACAAAGTATGGATGAAGGTCAGATTTTCTTG
TCAGTTTCTGAGAAACCTGGCAGCCTGCTGTTAACAACACAGGCCAGTATTGGGTTTTAT
TGAATTTGGTATGTGACCAAGGTCGGCCTAAAGGATGGCGCAGGTCCTGGGCAGGAAAGA
ATTTTTCCTTTATCACATAACTGTAATATTTGGTTGCTCAGCATAAGTGATGGAAGCAAA
CACTAATTTCTAATAAAATTGTGTTAAACTC
>gi|10863937|gb|NP_066960.1|TNFAIP1 316 aa linear tumor
necrosis factor, alpha-induced protein 1 [Homo sapiens].
MSGDTCLCPASGAKPKLSGFKGGGLGNKYVQLNVGGSLYYTTVRALTRHDTMLKAMFSGR
MEVLTDKEGWILIDRCGKHFGTILNYLRDDTITLPQNRQEIKELMAEAKYYLIQGLVNMC
QSALQDKKDSYQPVCNIPIITSLKEEERLIESSTKPVVKLLYNRSNNKYSYTSNSDDHLL
KNIELFDKLSLRFNGRVLFIKDVIGDEICCWSFYGQGRKLAEVCCTSIVYATEKKQTKVE
FPEARIYEETLNVLLYETPRVPDNSLLEATSRSRSQASPSEDEETPELRDRVRRIHVKRY
STYDDRQLGHQSTHRD
>gi|27597077|gb|NM_006293.2|TYRO3 3949 bp mRNA Homo sapiens
TYRO3 protein tyrosine kinase (TYRO3), mRNA.
GCGGTGGCGCGGGAGCGGCCCCGGGGACCCCGCGCTGCTGACGGCGGCGACCGCGGCCGG
AGGCGGGCGCGGGTCTCGGAGGCGGTCGCCTCAGCACCGCCCCACGGGCGGCCCCAGCCC
CTCCCGCAGCCCTCCTCCCTCCCGCTCCCTTCCCGCCGCCTCCTCCCCGCCCTCCTCCCT
CCTCGCTCGCGGGCCGGGCCCGGCATGGTGCGGCGTCGCCGCCGATGGCGCTGAGGCGGA
GCATGGGGCGGCCGGGGCTCCCGCCGCTGCCGCTGCCGCCGCCACCGCGGCTCGGGCTGC
TGCTGGCGGCTCTGGCTTCTCTGCTGCTCCCGGAGTCCGCCGCCGCAGGTCTGAAGCTCA
TGGGAGCCCCGGTGAAGCTGACAGTGTCTCAGGGGCAGCCGGTGAAGCTCAACTGCAGTG
TGGAGGGGATGGAGGAGCCTGACATCCAGTGGGTGAAGGATGGGGCTGTGGTCCAGAACT
TGGACCAGTTGTACATCCCAGTCAGCGAGCAGCACTGGATCGGCTTCCTCAGCCTGAAGT
CAGTGGAGCGCTCTGACGCCGGCCGGTACTGGTGCCAGGTGGAGGATGGGGGTGAAACCG
AGATCTCCCAGCCAGTGTGGCTCACGGTAGAAGGTGTGCCATTTTTCACAGTGGAGCCAA
AAGATCTGGCAGTGCCACCCAATGCCCCTTTCCAACTGTCTTGTGAGGCTGTGGGTCCCC
CTGAACCTGTTACCATTGTCTGGTGGAGAGGAACTACGAAGATCGGGGGACCCGCTCCCT
CTCCATCTGTTTTAAATGTAACAGGGGTGACCCAGAGCACCATGTTTTCCTGTGAAGCTC
ACAACCTAAAAGGCCTGGCCTCTTCTCGCACAGCCACTGTTCACCTTCAAGCACTGCCTG
CAGCCCCCTTCAACATCACCGTGACAAAGCTTTCCAGCAGCAACGCTAGTGTGGCCTGGA
TGCCAGGTGCTGATGGCCGAGCTCTGCTACAGTCCTGTACAGTTCAGGTGACACAGGCCC
CAGGAGGCTGGGAAGTCCTGGCTGTTGTGGTCCCTGTGCCCCCCTTTACCTGCCTGCTCC
GGGACCTGGTGCCTGCCACCAACTACAGCCTCAGGGTGCGCTGTGCCAATGCCTTGGGGC
CCTCTCCCTATGCTGACTGGGTGCCCTTTCAGACCAAGGGTCTAGCCCCAGCCAGCGCTC
CCCAAAACCTCCATGCCATCCGCACAGATTCAGGCCTCATCTTGGAGTGGGAAGAAGTGA
TCCCCGAGGCCCCTTTGGAAGGCCCCCTGGGACCCTACAAACTGTCCTGGGTTCAAGACA
ATGGAACCCAGGATGAGCTGACAGTGGAGGGGACCAGGGCCAATTTGACAGGCTGGGATC
CCCAAAAGGACCTGATCGTACGTGTGTGCGTCTCCAATGCAGTTGGCTGTGGACCCTGGA
GTCAGCCACTGGTGGTCTCTTCTCATGACCGTGCAGGCCAGCAGGGCCCTCCTCACAGCC
GCACATCCTGGGTACCTGTGGTCCTTGGTGTGCTAACGGCCCTGGTGACGGCTGCTGCCC
TGGCCCTCATCCTGCTTCGAAAGAGACGGAAAGAGACGCGGTTTGGGCAAGCCTTTGACA
GTGTCATGGCCCGGGGAGAGCCAGCCGTTCACTTCCGGGCAGCCCGGTCCTTCAATCGAG
AAAGGCCCGAGCGCATCGAGGCCACATTGGACAGCTTGGGCATCAGCGATGAACTAAAGG
AAAAACTGGAGGATGTGCTCATCCCAGAGCAGCAGTTCACCCTGGGCCGGATGTTGGGCA
AAGGAGAGTTTGGTTCAGTGCGGGAGGCCCAGCTGAAGCAAGAGGATGGCTCCTTTGTGA
AAGTGGCTGTGAAGATGCTGAAAGCTGACATCATTGCCTCAAGCGACATTGAAGAGTTCC
TCAGGGAAGCAGCTTGCATGAAGGAGTTTGACCATCCACACGTGGCCAAACTTGTTGGGG
TAAGCCTCCGGAGCAGGGCTAAAGGCCGTCTCCCCATCCCCATGGTCATCTTGCCCTTCA
TGAAGCATGGGGACCTGCATGCCTTCCTGCTCGCCTCCCGGATTGGGGAGAACCCCTTTA
ACCTACCCCTCCAGACCCTGATCCGGTTCATGGTGGACATTGCCTGCGGCATGGAGTACC
TGAGCTCTCGGAACTTCATCCACCGAGACCTGGCTGCTCGGAATTGCATGCTGGCAGAGG
ACATGACAGTGTGTGTGGCTGACTTCGGACTCTCCCGGAAGATCTACAGTGGGGACTACT
ATCGTCAAGGCTGTGCCTCCAAACTGCCTGTCAAGTGGCTGGCCCTGGAGAGCCTGGCCG
ACAACCTGTATACTGTGCAGAGTGACGTGTGGGCGTTCGGGGTGACCATGTGGGAGATCA
TGACACGTGGGCAGACGCCATATGCTGGCATCGAAAACGCTGAGATTTACAACTACCTCA
TTGGCGGGAACCGCCTGAAACAGCCTCCGGAGTGTATGGAGGACGTGTATGATCTCATGT
ACCAGTGCTGGAGTGCTGACCCCAAGCAGCGCCCGAGCTTTACTTGTCTGCGAATGGAAC
TGGAGAACATCTTGGGCCAGCTGTCTGTGCTATCTGCCAGCCAGGACCCCTTATACATCA
ACATCGAGAGAGCTGAGGAGCCCACTGCGGGAGGCAGCCTGGAGCTACCTGGCAGGGATC
AGCCCTACAGTGGGGCTGGGGATGGCAGTGGCATGGGGGCAGTGGGTGGCACTCCCAGTG
ACTGTCGGTACATACTCACCCCCGGAGGGCTGGCTGAGCAGCCAGGGCAGGCAGAGCACC
AGCCAGAGAGTCCCCTCAATGAGACACAGAGGCTTTTGCTGCTGCAGCAAGGGCTACTGC
CACACAGTAGCTGTTAGCCCACAGGCAGAGGGCATCGGGGCCATTTGGCCGGCTCTGGTG
GCCACTGAGCTGGCTGACTAAGCCCCGTCTGACCCCAGCCCAGACAGCAAGGTGTGGAGG
CTCCTGTGGTAGTCCTCCCAAGCTGTGCTGGGAAGCCCGGACTGACCAAATCACCCAATC
CCAGTTCTTCCTGCAACCACTCTGTGGCCAGCCTGGCATCAGTTTAGGCCTTGGCTTGAT
GGAAGTGGGCCAGTCCTGGTTGTCTGAACCCAGGCAGCTGGCAGGAGTGGGGTGGTTATG
TTTCCATGGTTACCATGGGTGTGGATGGCAGTGTGGGGAGGGCAGGTCCAGCTCTGTGGG
CCCTACCCTCCTGCTGAGCTGCCCCTGCTGCTTAAGTGCATGCATTGAGCTGCCTCCAGC
CTGGTGGCCCAGCTATTACCACACTTGGGGTTTAAATATCCAGGTGTGCCCCTCCAAGTC
ACAAAGAGATGTCCTTGTAATATTCCCTTTTAGGTGAGGGTTGGTAAGGGGTTGGTATCT
CAGGTCTGAATCTTCACCATCTTTCTGATTCCGCACCCTGCCTACGCCAGGAGAAGTTGA
GGGGAGCATGCTTCCCTGCAGCTGACCGGGTCACACAAAGGCATGCTGGAGTACCCAGCC
TATCAGGTGCCCCTCTTCCAAAGGCAGCGTGCCGAGCCAGCAAGAGGAAGGGGTGCTGTG
AGGCTTGCCCAGGAGCAAGTGAGGCCGGAGAGGAGTTCAGGAACCCTTCTCCATACCCAC
AATCTGAGCACGCTACCAAATCTCAAAATATCCTAAGACTAACAAAGGCAGCTGTGTCTG
AGCCCAACCCTTCTAAACGGTGACCTTTAGTGCCAACTTCCCCTCTAACTGGACAGCCTC
TTCTGTCCCAAGTCTCCAGAGAGAAATCAGGCCTGATGAGGGGGAATTCCTGGAACCTGG
ACCCCAGCCTTGGTGGGGGAGCCTCTGGAATGCATGGGGCGGGTCCTAGCTGTTAGGGAC
ATTTCCAAGCTGTTAGTTGCTGTTTAAAATAGAAATAAAATTGAAGACT
>gi|27597078|gb|NP_006284.2|TYRO3 890 aa linear TYRO3
protein tyrosine kinase; Brt; Dtk, Sky; Tif; Tyro3 protein
tyrosine kinase (sea-related receptor tyrosine kinase);
tyrosine-protein kinase receptor TYRO3 precursor [Homo
sapiens].
MALRRSMGRPGLPPLPLPPPPRLGLLLAALASLLLPESAAAGLKLMGAPVKLTVSQGQPV
KLNCSVEGMEEPDIQWVKDGAVVQNLDQLYIPVSEQHWIGFLSLKSVERSDAGRYWCQVE
DGGETEISQPVWLTVEGVPFFTVEPKDLAVPPNAPFQLSCEAVGPPEPVTIVWWRGTTKI
GGPAPSPSVLNVTGVTQSTMFSCEAHNLKGLASSRTATVHLQALPAAPFNITVTKLSSSN
ASVAWMPGADGRALLQSCTVQVTQAPGGWEVLAVVVPVPPFTCLLRDLVPATNYSLRVRC
ANALGPSPYADWVPFQTKGLAPASAPQNLHAIRTDSGLILEWEEVIPEAPLEGPLGPYKL
SWVQDNGTQDELTVEGTRANLTGWDPQKDLIVRVCVSNAVGCGPWSQPLVVSSHDRAGQQ
GPPHSRTSWVPVVLGVLTALVTAAALALILLRKRRKETRFGQAFDSVMARGEPAVHFRAA
RSFNRERPERIEATLDSLGISDELKEKLEDVLIPEQQFTLGRMLGKGEFGSVREAQLKQE
DGSFVKVAVKMLKADIIASSDIEEFLREAACMKEFDHPHVAKLVGVSLRSRAKGRLPIPM
VILPFMKHGDLHAFLLASRIGENPFNLPLQTLIRFMVDIACGMEYLSSRNFIHRDLAARN
CMLAEDMTVCVADFGLSRKIYSGDYYRQGCASKLPVKWLALESLADNLYTVQSDVWAFGV
TMWEIMTRGQTPYAGIENAEIYNYLIGGNRLKQPPECMEDVYDLMYQCWSADPKQRPSFT
CLRMELENILGQLSVLSASQDPLYINIERAEEPTAGGSLELPGRDQPYSGAGDGSGMGAV
GGTPSDCRYILTPGGLAEQPGQAEHQPESPLNETQRLLLLQQGLLPHSSC
>gi|4502884|gb|NM_003992.1|CLK3 1762 bp mRNA Homo sapiens
CDC-like kinase 3 (CLK3), transcript variant phclk3, mRNA.
TGGGGCACTGGTACCTCCAGGACCTGGAGTGTACTGGAAGAAATGGTGCAGTCCAGATGC
ATCACTGTAAGCGATACCGCTCCCCTGAACCAGACCCGTACCTGAGCTACCGATGGAAGA
GGAGGAGGTCCTACAGTCGGGAACATGAAGGGAGACTGCGATACCCGTCCCGAAGGGAGC
CTCCCCCACGAAGATCTCGGTCCAGAAGCCATGACCGCCTGCCCTACCAGAGGAGGTACC
GGGAGCGCCGTGACAGCGATACATACCGGTGTGAAGAGCGGAGCCCATCCTTTGGAGAGG
ACTACTATGGACCTTCACGTTCTCGTCATCGTCGGCGATCGCGGGAGAGGGGGCCATACC
GGACCCGCAAGCATGCCCACCACTGCCACAAACGCCGCACCAGGTCTTGTAGCAGCGCCT
CCTCGAGAAGCCAACAGAGCAGTAAGCGCACAGGCCGGAGTGTGGAAGATGACAAGGAGG
GTCACCTGGTGTGCCGGATCGGCGATTGGCTCCAAGAGCGATATGAGATTGTGGGGAACC
TGGGTGAAGGCACCTTTGGCAAGGTGGTGGAGTGCTTGGACCATGCCAGAGGGAAGTCTC
AGGTTGCCCTGAAGATCATCCGCAACGTGGGCAAGTACCGGGAGGCTGCCCGGCTAGAAA
TCAACGTGCTCAAAAAAATCAAGGAGAAGGACAAAGAAAACAAGTTCCTGTGTGTCTTGA
TGTCTGACTGGTTCAACTTCCACGGTCACATGTGCATCGCCTTTGAGCTCCTGGGCAAGA
ACACCTTTGAGTTCCTGAAGGAGAATAACTTCCAGCCTTACCCCCTACCACATGTCCGGC
ACATGGCCTACCAGCTCTGCCACGCCCTTAGATTTCTGCATGAGAATCAGCTGACCCATA
CAGACTTGAAACCTGAGAACATCCTGTTTGTGAATTCTGAGTTTGAAACCCTCTACAATG
AGCACAAGAGCTGTGAGGAGAAGTCAGTGAAGAACACCAGCATCCGAGTGGCTGACTTTG
GCAGTGCCACATTTGACCATGAGCACCACACCACCATTGTGGCCACCCGTCACTATCGCC
CGCCTGAGGTGATCCTTGAGCTGGGCTGGGCACAGCCCTGTGACGTCTGGAGCATTGGCT
GCATTCTCTTTGAGTACTACCGGGGCTTCACACTCTTCCAGACCCACGAAAACCGAGAGC
ACCTGGTGATGATGGAGAAGATCCTAGGGCCCATCCCATCACACATGATCCACCGTACCA
GGAAGCAGAAATATTTCTACAAAGGGGGCCTAGTTTGGGATGAGAACAGCTCTGACGGCC
GGTATGTGAAGGAGAACTGCAAACCTCTGAAGAGTTACATGCTCCAAGACTCCCTGGAGC
ACGTGCAGCTGTTTGACCTGATGAGGAGGATGTTAGAATTTGACCCTGCCCAGCGCATCA
CACTGGCCGAGGCCCTGCTGCACCCCTTCTTTGCTGGCCTGACCCCTGAGGAGCGGTCCT
TCCACACCAGCCGCAACCCAAGCAGATGACAGGCACAGGCCACCGCATGAGGAGATGGAG
GGCGGGACTGGGCCGCCCAGCCCCTTGACTCCAGCCTCGACCGCCAGCCCCAGGCCAGAG
CCACCCAATGAACAGTGCAATGTGAAGGAAGGCAGGAGCCTGCAGGGGAGCAGACTTGGT
GCCCAGCTGCCAGAAAGCACAGATTTGACCCAAGCTATTTATATGTTATAAAGTTATAAT
AAAGTGTTTCTTACTGTTTGTA
>gi|4502885|gb|NP_003983.1|CLK3 490 aa linear CDC-like
kinase 3 isoform hclk3 [Homo sapiens].
MHHCKRYRSPEPDPYLSYRWKRRRSYSREHEGRLRYPSRREPPPRRSRSRSHDRLPYQRR
YRERRDSDTYRCEERSPSFGEDYYGPSRSRHRRRSRERGPYRTRKHAHHCHKRRTRSCSS
ASSRSQQSSKRTGRSVEDDKEGHLVCRIGDWLQBRYEIVGNLGEGTFGKVVECLDHARGK
SQVALKIIRNVGKYREAARLEINVLKKIKEKDKENKFLCVLMSDWFNFHGHMCIAFELLG
KWTFEFLKENNFQPYPLPHVRHMAYQLCHALRFLHENQLTHTDLKPENILFVNSEFETLY
NEHKSCEEKSVKNTSIRVADFGSATFDHEHHTTIVATRHYRPPEVILELGWAQPCDVWSI
GCILFEYYRGFTLFQTHENREHLVMMEKILGPIPSHMIHRTRKQKYFYKGGLVWDENSSD
GRYVKENCKPLKSYMLQDSLEHVQLFDLMRRMLEFDPAQRITLAEALLHPFFAGLTPEER
SFHTSRNPSR
>gi|9910121|gb|NM_020249.1|ADAMTS9 3674 bp mRNA Homo sapiens
a disintegrin-like and metalloprotease (reprolysin type)
with thrombospondin type 1 motif, 9 (ADAMTS9), mRNA.
GCGGGAAGCACCATGCAGTTTGTATCCTGGGCCACACTGCTAACGCTCCTGGTGCGGGAC
CTGGCCGAGATGGGGAGCCCAGACGCCGCGGCGGCCGTACGCAAGGACAGGCTGCACCCG
AGGCAAGTGAAATTATTAGAGACCCTGGGCGAATACGAAATCGTGTCTCCCATCCGAGTG
AACGCTCTCGGAGAACCCTTTCCCACGAACGTCCACTTCAAAAGAACGCGACGGAGCATT
AACTCTGCCACTGACCCCTGGCCTGCCTTCGCCTCCTCCTCTTCCTCCTCTACCTCCTCC
CAGGCGCATTACCGCCTCTCTGCCTTCGGCCAGCAGTTTCTATTTAATCTCACCGCCAAT
GCCGGATTTATCGCTCCACTGTTCACTGTCACCCTCCTCGGGACGCCCGGGGTGAATCAG
ACCAAGTTTTATTCCGAAGAGGAAGCGGAACTCAAGCACTGTTTCTACAAAGGCTATGTC
AATACCAACTCCGAGCACACGGCCGTCATCAGCCTCTGCTCAGGAATGCTGGGCACATTC
CGGTCTCATGATGGGGATTATTTTATTGAACCACTACAGTCTATGGATGAACAAGAAGAT
GAAGAGGAACAAAACAAACCCCACATCATTTATAGGCGCAGCGCCCCCCAGAGAGAGCCC
TCAACAGGAAGGCATGCATGTGACACCTCAGAACACAAAAATAGGCACAGTAAAGACAAG
AAGAAAACCAGAGCAAGAAAATGGGGAGAAAGGATTAACCTGGCTGGTGACGTAGCAGCA
TTAAACAGCGGCTTAGCAACAGAGGCATTTTCTGCTTATGGTAATAAGACGGACAACACA
AGAGAAAAGAGGACCCACAGAAGGACAAAACGTTTTTTATCCTATCCACGGTTTGTAGAA
GTCTTGGTGGTGGCAGACAACAGAATGGTTTCATACCATGGAGAAAACCTTCAACACTAT
ATTTTAACTTTAATGTCAATTGTAGCCTCTATCTATAAAGACCCAAGTATTGGAAATTTA
ATTAATATTGTTATTGTGAACTTAATTGTGATTCATAATGAACAGGATGGGCCTTCCATA
TCTTTTAATGCTCAGACAACATTAAAAAACCTTTGCCAGTGGCAGCATTCGAAGAACAGT
CCAGGTGGAATCCATCATGATACTGCTGTTCTCTTAACAAGACAGGATATCTGCAGAGCT
CACGACAAATGTGATACCTTAGGCCTGGCTGAACTGGGAACCATTTGTGATCCCTATAGA
AGCTGTTCTATTAGTGAAGATAGTGGATTGAGTACAGCTTTTACGATCGCCCATGAGCTG
GGCCATGTGTTTAACATGCCTCATGATGACAACAACAAATGTAAAGAAGAAGGAGTTAAG
AGTCCCCAGCATGTCATGGCTCCAACACTGAACTTCTACACCAACCCCTGGATGTGGTCA
AAGTGTAGTCGAAAATATATCACTGAGTTTTTAGACACTGGTTATGGCGAGTGTTTGCTT
AACGAACCTGAATCCAGACCCTACCCTTTGCCTGTCCAACTGCCAGGCATCCTTTACAAC
GTGAATAAACAATGTGAATTGATTTTTGGACCAGGTTCTCAGGTGTGCCCATATATGATG
CAGTGCAGACGGCTCTGGTGCAATAACGTCAATGGAGTACACAAAGGCTGCCGGACTCAG
CACACACCCTGGGCCGATGGGACGGAGTGCGAGCCTGGAAAGCACTGCAAGTATGGATTT
TGTGTTCCCAAAGAAATGGATGTCCCCGTGACAGATGGATCCTGGGGAAGTTGGAGTCCC
TTTGGAACCTGCTCCAGAACATGTGGAGGGGGCATCAAAACAGCCATTCGAGAGTGCAAC
AGACCAGAACCAAAAAATGGTGGAAAATACTGTGTAGGACGTAGAATGAAATTTAAGTCC
TGCAACACGGAGCCATGTCTCAAGCAGAAGCGAGACTTCCGAGATGAACAGTGTGCTCAC
TTTGACGGGAAGCATTTTAACATCAACGGTCTGCTTCCCAATGTGCGCTGGGTCCCTAAA
TACAGTGGAATTCTGATGAAGGACCGGTGCAAGTTGTTCTGCAGAGTGGCAGGGAACACA
GCCTACTATCAGCTTCGAGACAGAGTGATAGATGGAACTCCTTGTGGCCAGGACACAAAT
GATATCTGTGTCCAGGGCCTTTGCCGGCAAGCTGGATGCGATCATGTTTTAAACTCAAAA
GCCCGGAGAGATAAATGTGGGGTTTGTGGTGGCGATAATTCTTCATGCAAAACAGTGGCA
GGAACATTTAATACAGTACATTATGGTTACAATACTGTGGTCCGAATTCCAGCTGGTGCT
ACCAATATTGATGTGCGGCAGCACAGTTTCTCAGGGGAAACAGACGATGACAACTACTTA
GCTTTATCAAGCAGTAAAGGTGAATTCTTGCTAAATGGAAACTTTGTTGTCACAATGGCC
AAAAGGGAAATTCGCATTGGGAATGCTGTGGTAGAGTACAGTGGGTCCGAGACTGCCGTA
GAAAGAATTAACTCAACAGATCGCATTGAGCAAGAACTTTTGCTTCAGGTTTTGTCGGTG
GGAAAGTTGTACAACCCCGATGTACGCTATTCTTTCAATATTCCAATTGAAGATAAACCT
CAGCAGTTTTACTGGAACAGTCATGGGCCATGGCAAGCATGCAGTAAACCCTGCCAAGGG
GAACGGAAACGAAAACTTGTTTGCACCAGGGAATCTGATCAGCTTACTGTTTCTGATCAA
AGATGCGATCGGCTGCCCCAGCCTGGACACATTACTGAACCCTGTGGTACAGACTGTGAC
CTGAGGTGGCATGTTGCCAGCAGGAGTGAATGTAGTGCCCAGTGTGGCTTGGGTTACCGC
ACATTGGACATCTACTGTGCCAAATATAGCAGGCTGGATGGGAAGACTGAGAAGGTTGAT
GATGGTTTTTGCAGCAGCCATCCCAAACCAAGCAACCGTGAAAAATGCTCAGGGGAATGT
AACACGGGTGGCTGGCGCTATTCTGCCTGGACTGAATGTTCAAAAAGCTGTGACGGTGGG
ACCCAGAGGAGAAGGGCTATTTGTGTCAATACCCGAAATGATGTACTGGATGACAGCAAA
TGCACACATCAAGAGAAAGTTACCATTCAGAGGTGCAGTGAGTTCCCTTGTCCACAGTGG
AAATCTGGAGACTGGTCAGAGGTAAGATGGGAGGGCTGTTATTTCCCCTAGGTCATCTCT
TACATTCTAGTTCTGGTGCTCTCTATCTGTTTAAGACAAACCCTTGTGCACCTTTCTCCC
ACCTCTCCCTTTCTCCCTTGTCTCCCTTGAGAAAACAACTCCAGTTCTCTGCCTGCACCA
TGACTGTCGTACTGGATGTAACTAGTCTACCAGTGACCTCAGGGCACTTTGGGCTTGGCT
AGATCACTCACTGTTGTAGCTTCTGTTGTGATTTTGAAGTTGCAGTCCATCACCTTCCCT
CCTCTTTGAGCCCTAGCTAAGTCACTGAAAGGAAATCATGGATTTATTAATCATAAAGCT
ATACTAGCTCACATCTGAAGTCAACATGAAGTTTCCTACTTCCTTGTCTTTGAAATAAGA
GAATTAGACCCCAGGGAGTGACCTCTCTGACTTACCCATCCAACTGCCCAAAAAAAAAAA
AAAAAAAAAAAAAA
>gi|99101122|gb|NP_064634.1|ADAMTS9 1072 aa linear a
disintegrin and metalloproteinase with thrombospondin
motifs-9 preproprotein [Homo sapiens].
MQFVSWATLLTLLVRDLAEMGSPDAAAAVRKDRLHPRQVKLLETLGEYEIVSPIRVNALG
EPFPTNVHFKRTRRSINSATDPWPAFASSSSSSTSSQAHYRLSAFGQQFLFNLTANAGFI
APLFTVTLLGTPGVNQTKFYSEEEAELKHCFYKGYVNTNSEHTAVISLCSGMLGTFRSHD
GDYFIEPLQSMDEQEDEEEQNKPHIIYRRSAPQREPSTGRHACDTSEHKNRHSKDKKKTR
ARKWGERINLAGDVAALNSGLATEAFSAYGNKTDNTREKRTHRRTKRFLSYPRFVEVLVV
ADNRMVSYHGENLQHYILTLMSIVASIYKDPSIGNLINIVIVNLIVIHNEQDGPSISFNA
QTTLKNLCQWQHSKNSPGGIHHDTAVLLTRQDICRAHDKCDTLGLAELGTICDPYRSCSI
SEDSGLSTAFTIAHELGHVFNMPHDDNNKCKEEGVKSPQHVMAPTLNFYTNPWMWSKCSR
KYITEFLDTGYGECLLNEPESRPYPLPVQLPGILYNVNKQCELIFGPGSQVCPYMMQCRR
LWCNNVNGVHKGCRTQHTPWADGTECEPGKHCKYGFCVPKEMDVPVTDGSWGSWSPFGTC
SRTCGGGIKTAIRECNRPEPKNGGKYCVGRRMKFKSCNTEPCLKQKRDFRDEQCAHFDGK
HFNINGLLPNVRWVPKYSGILMKDRCKLFCRVAGNTAYYQLRDRVIDGTPCGQDTNDICV
QGLCRQAGCDHVLNSKARRDKCGVCGGDNSSCKTVAGTFNTVHYGYNTVVRIPAGATNID
VRQHSFSGETDDDNYLALSSSKGEFLLNGNFVVTMAKREIRIGNAVVEYSGSETAVERIN
STDRIEQELLLQVLSVGKLYNPDVRYSFNIPIEDKPQQFYWNSHGPWQACSKPCQGERKR
KLVCTRESDQLTVSDQRCDRLPQPGHITEPCGTDCDLRWHVASRSECSAQCGLGYRTLDI
YCAKYSRLDGKTEKVDDGFCSSHPKPSNREKCSGECNTGGWRYSAWTECSKSCDGGTQRR
RAICVNTRNDVLDDSKCTHQEKVTIQRCSEFPCPQWKSGDWSEVRWEGCYFP
>gi|17981697|gb|NM_001262.2|CDKN2C 2104 bp mRNA Homo sapiens
cyclin-dependent kinase inhibitor 2C (p18, inhibits CDK4)
(CDKN2c), transcript variant 1, mRNA.
CTCTGCCGAGCCTCCTTAAAACTCTGCCGTTAAAATGGGGGCGGGTTTTTCAACTCAAAA
AGCGCTCAATTTTTTTCTTTTCAAAAAAAGCTGATGAGGTCGGAAAAAAGGGAGAAGAAA
CCGGCACCCTCTCTGAGAGGCAACAGAAGCAGCAATTGTTTCAGCGAAAAAAGCAGCAAG
GGAGGGAGTGAAGGAAAAAAGCAAAAAAGGGGGCGACACGCAAGTGCCTGTAGGGGTGAA
AGGAGCAGGGACCGGCGATCTAGGGGGGGATCAGCTACAAAAGAAACTGTCACTGGGAGC
GGTGCGGCCAAGGAGGAAGCAGTGCTGCCAGGCTCTGCTCCAGGGCACAGCTGGCTGGCG
GCTGCCCTGTCCGCAGCAAAGGGGCACAGGCCGGGGACCGCGAGAGGTGGCAAAGTGGCA
CCGGGCGCCGAGGCTGCTGAGCGCTCGCCGAGACGGCGACCGGACTGGCTGCCCCGGAAC
TGCGGCGACTCTCCCTACTCAGAACTTGGCCTACGTTTCCCAGGACTCTCCCCATCTCCA
GAGGCCCCCACAAAACCGGGAAAGGAAGGAAAGGACAGCGGCGGCAGCAGCTCAATGAGT
GCCTACAGCAGAAAGCCTGAACGAGCTCGGTCGTAGGCGGGAAGTTCCCGGGGGGGCTGC
CCAGTGCAGCCGCAATGCTGCCGCGAGCTGCCCCAGCAGTCCGGGCTCCGTAGACGCTTT
CCGCATCACTCTCCTTCCTCGGGCTGCCGGGAGTCCCGGGACCTGGCGGGGCCGGCATGA
CGGGCTTCTCGGGGGCCCGCCGCACGCCCGGCAGCCTCCGGAGACGCGCGCCGAGCCCGG
CTCCCACGGCCTCTGAGGCTCGGCGGGGCTGCGGCTGCCTGGCGGGCGGGCTCCGGAGCT
TTCCTGAGCGGCATTAGCCCACGGCTTGGCCCGGACGCGACCAAAGGCTCTTCTGGAGAA
GCCCAGAGCACTGGGCAATCGTTACGACCTGTAACTTGAGGGCCACCGAACTGCTACTCC
CGTTCGCCTTTGGCGATCATCTTTTAACCCTCCGGAGCACGTCAGCATCCAGCCACCGCG
GCGCTCTCCCAGCAGCGGAGGACCCAGGACTATCCCTTCGGCGAGACGGATGGAAACCGA
GCCCCCTGGAGGACCTGCCCCTGCAGTTCTGCCTCACACGGCTCAAGTCACCACCGTGAA
CAAGGGACCCTAAAGAATGGCCGAGCCTTGGGGGAACGAGTTGGCGTCCGCAGCTGCCAG
GGGGGACCTAGAGCAACTTACTAGTTTGTTGCAAAATAATGTAAACGTCAATGCACAAAA
TGGATTTGGAAGGACTGCGCTGCAGGTTATGAAACTTGGAAATCCCGAGATTGCCAGGAG
ACTGCTACTTAGAGGTGCTAATCCCGATTTGAAAGACCGAACTGGTTTCGCTGTCATTCA
TGATGCGGCCAGAGCAGGTTTCCTGGACACTTTACAGACTTTGCTGGAGTTTCAAGCTGA
TGTTAACATCGAGGATAATGAAGGGAACCTGCCCTTGCACTTGGCTGCCAAAGAAGGCCA
CCTCCGGGTGGTGGAGTTCCTGGTGAAGCACACGGCCAGCAATGTGGGGCATCGGAACCA
TAAGGGGGACACCGCCTGTGATTTGGCCAGGCTCTATGGGAGGAATGAGGTTGTTAGCCT
GATGCAGGCAAACGGGGCTGGGGGAGCCACAAATCTTCAATAAACGTGGGGAGGGCTCCC
CCACGTTGCCTCTACTTTATCAATTAACTGAGTAGCTCTCCTGACTTTTAATGTCATTTG
TTAAAATACAGTTCTGTCATATGTTAAGCAGCTAAATTTTCTGAAACTGCATAAGTGAAA
ATCTTACAACAGGCTTATGAATATATTTAAGCAACATCTTTTTAACCTGCAAAATCTGTT
CTAACATGTAATTGCAGATAACTTTGACTTTCTTCTGAATATTTTATCTTTCCTTGGCTT
TTCCCTTGCTTCCCCTTTTGCCAATCTCAACACCCAAGTTGAAGACTTTGTTTTTAAAAT
GGTTTGTCCTGATGCTTTTGTCTAATTAAAACACTTTCAAAACAGGAAAAAAAAAAAAAA
AAAA
>gi|4502751|gb|NP_001253.1|CDKN2C 168 aa linear cyclin-
dependent kinase inhibitor 2C; cyclin-dependent kinase 6
inhibitor p18; cyclin-dependent kinase 4 inhibitor C;
cyclin-dependent inhibitor; CDK6 inhibitor p18 [Homo
sapiens].
MAEPWGNELASAAARGDLEQLTSLLQNNVNVNAQNGFGRTALQVMKLGNPEIARRLLLRG
ANPDLKDRTGFAVIHDAARAGFLDTLQTLLEFQADVNIEDNEGNLPLHLAAKEGHLRVVE
FLVKHTASNVGHRNHKGDTACDLARLYGRNEVVSLMQANGAGGATNLQ
>gi|23510344|gb|NM_002037.3|FYN 2650 bp mRNA Homo sapiens
FYN oncogene related to SRC, FGR, YES (FYN), transcript
variant 1, mRNA.
GCCGCGCTGGTGGCGGCGGCGCGTCGTTGCAGTTGCGCCATCTGTCAGGAGCGGAGCCGG
CGAGGAGGGGGCTGCCGCGGGCGAGGAGGAGGGGTCGCCGCGAGCCGAAGGCCTTCGAGA
CCCGCCCGCCGCCCGGCGGCGAGAGTAGAGGCGAGGTTGTTGTGCGAGCGGCGCGTCCTC
TCCCGCCCGGGCGCGCCGCGCTTCTCCCAGCGCACCGAGGACCGCCCGGGCGCACACAAA
GCCGCCGCCCGCGCCGCACCGCCCGGCGGCCGCCGCCCGCGCCAGGGAGGGATTCGGCCG
CCGGGCCGGGGACACCCCGGCGCCGCCCCCTCGGTGCTCTCGGAAGGCCCACCGGCTCCC
GGGCCCGCCGGGGACCCCCCGGAGCCGCCTCGGCCGCGCCGGAGGAGGGCGGGGAGAGGA
CCATGTGAGTGGGCTCCGGAGCCTCAGCGCCGCGCAGTTTTTTTGAAGAAGCAGGATGCT
GATCTAAACGTGGAAAAAGACCAGTCCTGCCTCTGTTGTAGAAGACATGTGGTGTATATA
AAGTTTGTGATCGTTGGCGGACATTTTGGAATTTAGATAATGGGCTGTGTGCAATGTAAG
GATAAAGAAGCAACAAAACTGACGGAGGAGAGGGACGGCAGCCTGAACCAGAGCTCTGGG
TACCGCTATGGCACAGACCCCACCCCTCAGCACTACCCCAGCTTCGGTGTGACCTCCATC
CCCAACTACAACAACTTCCACGCAGCCGGGGGCCAAGGACTCACCGTCTTTGGAGGTGTG
AACTCTTCGTCTCATACGGGGACCTTGCGTACGAGAGGAGGAACAGGAGTGACACTCTTT
GTGGCCCTTTATGACTATGAAGCACGGACAGAAGATGACCTGAGTTTTCACAAAGGAGAA
AAATTTCAAATATTGAACAGCTCGGAAGGAGATTGGTGGGAAGCCCGCTCCTTGACAACT
GGAGAGACAGGTTACATTCCCAGCAATTATGTGGCTCCAGTTGACTCTATCCAGGCAGAA
GAGTGGTACTTTGGAAAACTTGGCCGAAAAGATGCTGAGCGACAGCTATTGTCCTTTGGA
AACCCAAGAGGTACCTTTCTTATCCGCGAGAGTGAAACCACCAAAGGTGCCTATTCACTT
TCTATCCGTGATTGGGATGATATGAAAGGAGACCATGTCAAACATTATAAAATTCGCAAA
CTTGACAATGGTGGATACTACATTACCACCCGGGCCCAGTTTGAAACACTTCAGCAGCTT
GTACAACATTACTCAGAGAGAGCTGCAGGTCTCTGCTGCCGCCTAGTAGTTCCCTGTCAC
AAAGGGATGCCAAGGCTTACCGATCTGTCTGTCAAAACCAAAGATGTCTGGGAAATCCCT
CGAGAATCCCTGCAGTTGATCAAGAGACTGGGAAATGGGCAGTTTGGGGAAGTATGGATG
GGTACCTGGAATGGAAACACAAAAGTAGCCATAAAGACTCTTAAACCAGGCACAATGTCC
CCCGAATCATTCCTTGAGGAAGCGCAGATCATGAAGAAGCTGAAGCACGACAAGCTGGTC
CAGCTCTATGCAGTGGTGTCTGAGGAGCCCATCTACATCGTCACCGAGTATATGAACAAA
GGAAGTTTACTGGATTTCTTAAAAGATGGAGAAGGAAGAGCTCTGAAATTACCAAATCTT
GTGGACATGGCAGCACAGGTGGCTGCAGGAATGGCTTACATCGAGCGCATGAATTATATC
CATAGAGATCTGCGATCAGCAAACATTCTAGTGGGGAATGGACTCATATGCAAGATTGCT
GACTTCGGATTGGCCCGATTGATAGAAGACAATGAGTACACAGCAAGACAAGGTGCAAAG
TTCCCCATCAAGTGGACGGCCCCCGAGGCAGCCCTGTACGGGAGGTTCACAATCAAGTCT
GACGTGTGGTCTTTTGGAATCTTACTCACAGAGCTGGTCACCAAAGGAAGAGTGCCATAC
CCAGGCATGAACAACCGGGAGGTGCTGGAGCAGGTGGAGCGAGGCTACAGGATGCCCTGC
CCGCAGGACTGCCCCATCTCTCTGCATGAGCTCATGATCCACTGCTGGAAAAAGGACCCT
GAAGAACGCCCCACTTTTGAGTACTTGCAGAGCTTCCTGGAAGACTACTTTACCGCGACA
GAGCCCCAGTACCAACCTGGTGAAAACCTGTAAGGCCCGGGTCTGCGGAGAGAGGCCTTG
TCCCAGAGGCTGCCCCACCCCTCCCCATTAGCTTTCAATTCCGTAGCCAGCTGCTCCCCA
GCAGCGGAACCGCCCAGGATCAGATTGCATGTGACTCTGAAGCTGACGAACTTCCATGGC
CCTCATTAATGACACTTGTCCCCAAATCCGAACCTCCTCTGTGAAGCATTCGAGACAGAA
CCTTGTTATTTCTCAGACTTTGGAAAATGCATTGTATCGATGTTATGTAAAAGGCCAAAC
CTCTGTTCAGTGTAAATAGTTACTCCAGTGCCAACAATCCTAGTGCTTTCCTTTTTTAAA
AATGCAAATCCTATGTGATTTTAACTCTGTCTTCACCTGATTCAACTAAAAAAAAAAAAG
TATTATTTTCCAAAAGTGGCCTCTTTGTCTAAAACAATAAAATTTTTTTTCATGTTTTAA
CAAAAACCAA
>gi|4503823|gb|NP_002028.1|FYN 537 aa linear protein-
tyrosine kinase fyn isoform a; proto-oncogene tyrosine-
protein kinase fyn; src/yes-related novel gene; src-like
kinase; c-syn protooncogene; tyrosine kinase p59fyn(T);
OKT3-induced calcium influx regulator [Homo sapiens].
MGCVQCKDKEATKLTEERDGSLNQSSGYRYGTDPTPQHYPSFGVTSIPNYNNFHAAGGQG
LTVFGGVNSSSHTGTLRTRGGTGVTLFVALYDYEARTEDDLSFHKGEKFQILNSSEGDWW
EARSLTTGETGYIPSNYVAPVDSIQAEEWYFGKLGRKDAERQLLSFGNPRGTFLIRESET
TKGAYSLSIRDWDDMKGDHVKHYKIRKLDNGGYYITTRAQFETLQQLVQHYSERAAGLCC
RLVVPCHKGMPRLTDLSVKTKDVWEIPRESLQLIKRLGNGQFGEVWMGTWNGNTKVAIKT
LKPGTMSPESFLEEAQIMKKLKHDKLVQLYAVVSEEPIYIVTEYMNKGSLLDFLKDGEGR
ALKLPNLVDMAAQVAAGMAYIERMNYIHRDLRSANILVGNGLICKIADFGLARLIEDNEY
TARQGAKFPIKWTAPEAALYGRFTIKSDVWSFGILLTELVTKGRVPYPGMNNREVLEQVE
RGYRMPCPQDCPISLHELMIHCWKKDPEERPTFEYLQSFLEDYFTATEPQYQPGENL
>gi|15055546|gb|NM_000800.2|FGF1 2357 bp mRNA Homo sapiens
fibroblast growth factor 1 (acidic) (FGF1), transcript
variant 1, mRNA.
GAGCCGGGCTACTCTGAGAAGAAGACACCAAGTGGATTCTGCTTCCCCTGGGACAGCACT
GAGCGAGTGTGGAGAGAGGTACAGCCCTCGGCCTACAAGCTCTTTAGTCTTGAAAGCGCC
ACAAGCAGCAGCTGCTGAGCCATGGCTGAAGGGGAAATCACCACCTTCACAGCCCTGACC
GAGAAGTTTAATCTGCCTCCAGGGAATTACAAGAAGCCCAAACTCCTCTACTGTAGCAAC
GGGGGCCACTTCCTGAGGATCCTTCCGGATGGCACAGTGGATGGGACAAGGGACAGGAGC
GACCAGCACATTCAGCTGCAGCTCAGTGCGGAAAGCGTGGGGGAGGTGTATATAAAGAGT
ACCGAGACTGGCCAGTACTTGGCCATGGACACCGACGGGCTTTTATACGGCTCACAGACA
CCAAATGAGGAATGTTTGTTCCTGGAAAGGCTGGAGGAGAACCATTACAACACCTATATA
TCCAAGAAGCATGCAGAGAAGAATTGGTTTGTTGGCCTCAAGAAGAATGGGAGCTGCAAA
CGCGGTCCTCGGACTCACTATGGCCAGAAAGCAATCTTGTTTCTCCCCCTGCCAGTCTCT
TCTGATTAAAGAGATCTGTTCTGGGTGTTGACCACTCCAGAGAAGTTTCGAGGGGTCCTC
ACCTGGTTGACCCAAAAATGTTCCCTTGACCATTGGCTGCGCTAACCCCCAGCCCACAGA
GCCTGAATTTGTAAGCAACTTGCTTCTAAATGCCCAGTTCACTTCTTTGCAGAGCCTTTT
ACCCCTGCACAGTTTAGAACAGAGGGACCAAATTGCTTCTAGGAGTCAACTGGCTGGCCA
GTCTGGGTCTGGGTTTGGATCTCCAATTGCCTCTTGCAGGCTGAGTCCCTCCATGCAAAA
GTGGGGCTAAATGAAGTGTGTTAAGGGGTCGGCTAAGTGGGACATTAGTAACTGCACACT
ATTTCCCTCTACTGAGTAAACCCTATCTGTGATTCCCCCAAACATCTGGCATGGCTCCCT
TTTGTCCTTCCTGTGCCCTGCAAATATTAGCAAAGAAGCTTCATGCCAGGTTAGGAAGGC
AGCATTCCATGACCAGAAACAGGGACAAAGAAATCCCCCCTTCAGAACAGAGGCATTTAA
AATGGAAAAGAGAGATTGGATTTTGGTGGGTAACTTAGAAGGATGGCATCTCCATGTAGA
ATAAATGAAGAAAGGGAGGCCCAGCCGCAGGAAGGCAGAATAAATCCTTGGGAGTCATTA
CCACGCCTTGACCTTCCCAAGGTTACTCAGCAGCAGAGAGCCCTGGGTGACTTCAGGTGG
AGAGCACTAGAAGTGGTTTCCTGATAACAAGCAAGGATATCAGAGCTGGGAAATTCATGT
GGATCTGGGGACTGAGTGTGGGAGTGCAGAGAAAGAAAGGGAAACTGGCTGAGGGGATAC
CATAAAAAGAGGATGATTTCAGAAGGAGAAGGAAAAAGAAAGTAATGCCACACATTGTGC
TTGGCCCCTGGTAAGCAGAGGCTTTGGGGTCCTAGCCCAGTGCTTCTCCAACACTGAAGT
GCTTGCAGATCATCTGGGGACCTGGTTTGAATGGAGATTCTGATTCAGTGGGTTGGGGGC
AGAGTTTCTGCAGTTCCATCAGGTCCCCCCCAGGTGCAGGTGCTGACAATACTGCTGCCT
TACCCGCCATACATTAAGGAGCAGGGTCCTGGTCCTAAAGAGTTATTCAAATGAAGGTGG
TTCGACGCCCCGAACCTCACCTGACCTCAACTAACCCTTAAAAATGCACACCTCATGAGT
CTACCTGAGCATTCAGGCAGCACTGACAATAGTTATGCCTGTACTAAGGAGCATGATTTT
AAGAGGCTTTGGCCAATGCCTATAAAATGCCCATTTCGAAGATATACAAAAACATACTTC
AAAAATGTTAAACCCTTACCAACAGCTTTTCCCAGGAGACCATTTGTATTACCATTACTT
GTATAAATACACTTCCTGCTTAAACTTGACCCAGGTGGCTAGCAAATTAGAAACACCATT
CATCTCTAACATATGATACTGATGCCATGTAAAGGCCTTTAATAAGTCATTGAAATTTAC
TGTGAGACTGTATGTTTTAATTGCATTTAAAAATATATAGCTTGAAAGCAGTTAAACTGA
TTAGTATTCAGGCACTGAGAATGATAGTAATAGGATACAATGTATAAGCTACTCACTTAT
CTGATACTTATTTACCTATAAAATGAGATTTTTGTTTTCCACTGTGCTATTACAAATTTT
CTTTTGAAAGTAGGAACTCTTAAGCAATGGTAATTGTGAATAAAAATTGATGAGAGTGTT
AAAAAAAAAAAAAAAAA
>gi|4503697|gb|NP_000791.1|FGF1 155 aa linear fibroblast
growth factor 1 (acidic) isoform 1 precursor; heparin-
binding growth factor 1 precursor; endothelial cell growth
factor, alpha; endothelial cell growth factor, beta [Homo
sapiens].
MAEGEITTFTALTEKFNLPPGNYKKPKLLYCSNGGHFLRILPDGTVDGTRDRSDQHIQLQ
LSAESVGEVYIKSTETGQYLAMDTDGLLYGSQTPNEECLFLERLEENHYNTYISKKHAEK
NWFVGLKKNGSCKRGPRTHYGQKAILFLPLPVSSD
>gi|27552761|gb|NM_002825.3|PTN 1029 bp mRNA Homo sapiens
pleiotrophin (heparin binding growth factor 8, neurite
growth-promoting factor 1) (PTN), mRNA.
TCTGCTTTTAATAAGCTTCCCAATCAGCTCTCGAGTGCAAAGCGCTCTCCCTCCCTCGCC
CAGCCTTCGTCCTCCTGGCCCGCTCCTCTCATCCCTCCCATTCTCCATTTCCCTTCCGTT
CCCTCCCTGTCAGGGCGTAATTGAGTCAAAGGCAGGATCAGGTTCCCCGCCTTCCAGTCC
AAAAATCCCGCCAAGAGAGCCCCAGAGCAGAGGAAAATCCAAAGTGGAGAGAGGGGAAGA
AAGAGACCAGTGAGTCATCCGTCCAGAAGGCGGGGAGAGCAGCAGCGGCCCAAGCAGGAG
CTGCAGCGAGCCGGGTACCTGGACTCAGCGGTAGCAACCTCGCCCCTTGCAACAAAGGCA
GACTGAGCGCCAGAGAGGACGTTTCCAACTCAAAAATGCAGGCTCAACAGTACCAGCAGC
AGCGTCGAAAATTTGCAGCTGCCTTCTTGGCATTCATTTTCATACTGGCAGCTGTGGATA
CTGCTGAAGCAGGGAAGAAAGAGAAACCAGAAAAAAAAGTGAAGAAGTCTGACTGTGGAG
AATGGCAGTGGAGTGTGTGTGTGCCCACCAGTGGAGACTGTGGGCTGGGCACACGGGAGG
GCACTCGGACTGGAGCTGAGTGCAAGCAAACCATGAAGACCCAGAGATGTAAGATCCCCT
GCAACTGGAAGAAGCAATTTGGCGCGGAGTGCAAATACCAGTTCCAGGCCTGGGGAGAAT
GTGACCTGAACACAGCCCTGAAGACCAGAACTGGAAGTCTGAAGCGAGCCCTGCACAATG
CCGAATGCCAGAAGACTGTCACCATCTCCAAGCCCTGTGGCAAACTGACCAAGCCCAAAC
CTCAAGCAGAATCTAAGAAGAAGAAAAAGGAAGGCAAGAAACAGGAGAAGATGCTGGATT
AAAAGATGTCACCTGTGGAACATAAAAAGGACATCAGCAAACAGGATCAGTTAACTATTG
CATTTATATGTACCGTAGGCTTTGTATTCAAAAATTATCTATAGCTAAGTACACAATAAG
CAAAAACAA
>gi|4506281|gb|NP_002816.1|PTN 168 aa linear pleiotrophin
(heparin binding growth factor 8, neurite growth-promoting
factor 1); heparin affin regulatory protein; heparin-binding
growth-associated molecule [Homo sapiens].
MQAQQYQQQRRKFAAAFLAFIFILAAVDTAEAGKKEKPEKKVKKSDCGEWQWSVCVPTSG
DCGLGTREGTRTGAECKQTMKTQRCKIPCNWKKQFGAECKYQFQAWGECDLNTALKTRTG
SLKRALHNAECQKTVTISKPCGKLTKPKPQAESKKKKKEGKKQEKMLD
>gi|4504008|gb|NM_000169.1|GLA 1350 bp mRNA Homo sapiens
galactosidase, alpha (GLA), mRNA.
AGGTTAATCTTAAAAGCCCAGGTTACCCGCGGAAATTTATGCTGTCCGGTCACCGTGACA
ATGCAGCTGAGGAACCCAGAACTACATCTGGGCTGCGCGCTTGCGCTTCGCTTCCTGGCC
CTCGTTTCCTGGGACATCCCTGGGGCTAGAGCACTGGACAATGGATTGGCAAGGACGCCT
ACCATGGGCTGGCTGCACTGGGAGCGCTTCATGTGCAACCTTGACTGCCAGGAAGAGCCA
GATTCCTGCATCAGTGAGAAGCTCTTCATGGAGATGGCAGAGCTCATGGTCTCAGAAGGC
TGGAAGGATGCAGGTTATGAGTACCTCTGCATTGATGACTGTTGGATGGCTCCCCAAAGA
GATTCAGAAGGCAGACTTCAGGCAGACCCTCAGCGCTTTCCTCATGGGATTCGCCAGCTA
GCTAATTATGTTCACAGCAAAGGACTGAAGCTAGGGATTTATGCAGATGTTGGAAATAAA
ACCTGCGCAGGCTTCCCTGGGAGTTTTGGATACTACGACATTGATGCCCAGACCTTTGCT
GACTGGGGAGTAGATCTGCTAAAATTTGATGGTTGTTACTGTGACAGTTTGGAAAATTTG
GCAGATGGTTATAAGCACATGTCCTTGGCCCTGAATAGGACTGGCAGAAGCATTGTGTAC
TCCTGTGAGTGGCCTCTTTATATGTGGCCCTTTCAAAAGCCCAATTATACAGAAATCCGA
CAGTACTGCAATCACTGGCGAAATTTTGCTGACATTGATGATTCCTGGAAAAGTATAAAG
AGTATCTTGGACTGGACATCTTTTAACCAGGAGAGAATTGTTGATGTTGCTGGACCAGGG
GGTTGGAATGACCCAGATATGTTAGTGATTGGCAACTTTGGCCTCAGCTGGAATCAGCAA
GTAACTCAGATGGCCCTCTGGGCTATCATGGCTGCTCCTTTATTCATGTCTAATGACCTC
CGACACATCAGCCCTCAAGCCAAAGCTCTCCTTCAGGATAAGGACGTAATTGCCATCAAT
CAGGACCCCTTGGGCAAGCAAGGGTACCAGCTTAGACAGGGAGACAACTTTGAAGTGTGG
GAACGACCTCTCTCAGGCTTAGCCTGGGCTGTAGCTATGATAAACCGGCAGGAGATTGGT
GGACCTCGCTCTTATACCATCGCAGTTGCTTCCCTGGGTAAAGGAGTGGCCTGTAATCCT
GCCTGCTTCATCACACAGCTCCTCCCTGTGAAAAGGAAGCTAGGGTTCTATGAATGGACT
TCAAGGTTAAGAAGTCACATAAATCCCACAGGCACTGTTTTGCTTCAGCTAGAAAATACA
ATGCAGATGTCATTAAAAGACTTACTTTAA
>gi|4504009|gb|NP_000160.1|GLA 429 aa linear galactosidase,
alpha [Homo sapiens].
MQLRNPELHLGCALALRFLALVSWDIPGARALDNGLARTPTMGWLHWERFMCNLDCQEEP
DSCISEKLFMEMAELMVSEGWKDAGYEYLCIDDCWMAPQRDSEGRLQADPQRFPHGIRQL
ANYVHSKGLKLGIYADVGNKTCAGFPGSFGYYDIDAQTFADWGVDLLKFDGCYCDSLENL
ADGYKHMSLALNRTGRSIVYSCEQPLYMWPFQKPNYTEIRQYCNHWRNFADIDDSWKSIK
SILDWTSFNQERIVDVAGPGGWNDPDMLVIGNFGLSWNQQVTQMALWAIMAAPLFMSNDL
RHISPQAKALLQDKDVIAINQDPLGKQGYQLRQGDNFEVWERPLSGLAWAVAMINRQEIG
GPRSYTIAVASLGKGVACNPACFITQLLPVKRKLGFYEWTSRLRSHINPTGTVLLQLENT
MQMSLKDLL
>gi|18587778|gb|XM_091624.1|LOC162542 287 bp mRNA Homo
sapiens similar to ADP-ribosylation factor 1 (LOC162542),
mRNA.
GTCTGATTTTTATGGTTGACAGTAATGACAGAGAGCAGATTGATGAGGCCTGGGAAGTGC
TAACTTACTTGTTAGAGGACGATGAGCTCAGAAATGCAGTTTTATTGGTATTTGCCAATA
AACAAGATCTCCCTAATACTATGAACGCGGCAGAGATAACGGACAAGCTCGGCCTCCATT
CCCTCCGCTACAGAAACTGGCACATTCAGGCTACTTGTGCCACTACTGGACATGGGCTTT
ACGAAGGCCTGAACTGGCTCGCCAACCAGTTCCAGAACCAGAACTGA
>gi|18587779|gb|XP_091624.1|LOC162542 91 aa linear similar
to ADP-ribosylation factor 1 [Homo sapiens].
MVDSNDREQIDEAWEVLTYLLEDDELRNAVLLVFANKQDLPNTMNAAEITDKLGLHSLRY
RNWHIQATCATTGHGLYEGLNWLANQFQNQN
>gi|4557572|gb|NM_000401.1|EXT2 3781 bp mRNA Homo sapiens
exostoses (multiple) 2 (EXT2), mRNA.
CTGTCTGAGCATTTCACTGCGGAGCCTGAGCGCGCCTGCCTGGGAAAACACTGCAGCGGT
GCTCGGACTCCTCCTGTCCAGCAGGAGGCGCGGCCCGGCAGCTCCCGCATGCGCAGTGCG
CTCGGTGTCAGACGGCCCGGATCCCGGTTACCGGCCCCTCGCTCGCTGCTCGCCAGCCCA
GACTCGGCCCTGGCAGTGGCGGCTGGCGATTCGGACCGATCCGACCTGGGCGGAGGTGGC
CCGCGCCCCGCGGCATGAGCCGGTGACCAAGCTCGGGGCCGAGCGGGAGGCAGCCGTGGC
CGAGCCACAGGGATCTGATTCCTCCCAGGGGGATGTCCTGCGCCTCAGGGTCCGGTGGTG
GCCTGCGGCATCCCTTGCGGTGCCAGAAGCCGTGGGACGAGTGTCTTTAATGTTATAGAG
CTACTCAGAGTTGCTGTTTCTCCTTGAGATGCTTTTGGAGTGTGAGGAAGAGGCTGTCTG
TGTCATTATGTGTGCGTCGGTCAAGTATAATATCCGGGGTCCTGCCCTCATCCCAAGAAT
GAAGACCAAGCACCGAATCTACTATATCACCCTCTTCTCCATTGTCCTCCTGGGCCTCAT
TGCCACTGGCATGTTTCAGTTTTGGCCCCATTCTATCGAGTCCTCAAATGACTGGAATGT
AGAGAAGCGCAGCATCCGTGATGTGCCGGTTGTTAGGCTGCCAGCCGACAGTCCCATCCC
AGAGCGGGGGGATCTCAGTTGCAGAATGCACACGTGTTTTGATGTCTATCGCTGTGGCTT
CAACCCAAAGAACAAAATCAAGGTGTATATCTATGCTCTGAAAAAGTACGTGGATGACTT
TGGCGTCTCTGTCAGCAACACCATCTCCCGGGAGTATAATGAACTGCTCATGGCCATCTC
AGACAGTGACTACTACACTGATGACATCAACCGGGCCTGTCTGTTTGTTCCCTCCATCGA
TGTGCTTAACCAGAACACACTGCGCATCAAGGAGACAGCACAAGCGATGGCCCAGCTCTC
TAGGTGGGATCGAGGTACGAATCACCTGTTGTTCAACATGTTGCCTGGAGGTCCCCCAGA
TTATAACACAGCCCTGGATGTCCCCAGAGACAGGGCCCTGTTGGCTGGTGGCGGCTTTTC
TACGTGGACTTACCGGCAAGGCTACGATGTCAGCATTCCTGTCTATAGTCCACTGTCAGC
TGAGGTGGATCTTCCAGAGAAAGGACCAGGTCCACGGCAATACTTCCTCCTGTCATCTCA
GGTGGGTCTCCATCCTGAGTACAGAGAGGACCTAGAAGCCCTCCAGGTCAAACATGGAGA
GTCAGTGTTAGTACTCGATAAATGCACCAACCTCTCAGAGGGTGTCCTTTCTGTCCGTAA
GCGCTGCCACAAGCACCAGGTCTTCGATTACCCACAGGTGCTACAGGAGGCTACTTTCTG
TGTGGTTCTTCGTGGAGCTCGGCTGGGCCAGGCAGTATTGAGCGATGTGTTACAAGCTGG
CTGTGTCCCGGTTGTCATTGCAGACTCCTATATTTTGCCTTTCTCTGAAGTTCTTGACTG
GAAGAGAGCATCTGTGGTTGTACCAGAAGAAAAGATGTCAGATGTGTACAGTATTTTGCA
GAGCATCCCCCAAAGACAGATTGAAGAAATGCAGAGACAGGCCCGGTGGTTCTGGGAAGC
GTACTTCCAGTCAATTAAAGCCATTGCCCTGGCCACCCTGCAGATTATCAATGACCGGAT
CTATCCATATGCTGCCATCTCCTATGAAGAATGGAATGACCCTCCTGCTGTGAAGTGGGG
CAGCGTGAGCAATCCACTCTTCCTCCCGCTGATCCCACCACAGTCTCAAGGGTTCACCGC
CATAGTCCTCACCTACGACCGAGTAGAGAGCCTCTTCCGGGTCATCACTGAAGTGTCCAA
GGTGCCCAGTCTATCCAAACTACTTGTCGTCTGGAATAATCAGAATAAAAACCCTCCAGA
AGATTCTCTCTGGCCCAAAATCCGGGTTCCATTAAAAGTTGTGAGGACTGCTGAAAACAA
GTTAAGTAACCGTTTCTTCCCTTATGATGAAATCGAGACAGAAGCTGTTCTGGCCATTGA
TGATGATATCATTATGCTGACCTCTGACGAGCTGCAATTTGGTTATGAGGTCTGGCGGGA
ATTTCCTGACCGGTTGGTGGGTTACCCGGGTCGTCTGCATCTCTGGGACCATGAGATGAA
TAAGTGGAAGTATGAGTCTGAGTGGACGAATGAAGTGTCCATGGTGCTCACTGGGGCAGC
TTTTTATCACAAGTATTTTAATTACCTGTATACCTACAAAATGCCTGGGGATATCAAGAA
CTGGGTAGATGCTCATATGAACTGTGAAGATATTGCCATGAACTTCCTGGTGGCCAACGT
CACGGGAAAAGCAGTTATCAAGGTAACCCCACGAAAGAAATTCAAGTGTCCTGAGTGCAC
AGCCATAGATGGGCTTTCACTAGACCAAACACACATGGTGGAGAGGTCAGAGTGCATCAA
CAAGTTTGCTTCAGTCTTCGGGACCATGCCTCTCAAGGTGGTGGAACACCGAGCTGACCC
TGTCCTGTACAAAGATGACTTTCCTGAGAAGCTGAAGAGCTTCCCCAACATTGGCAGCTT
ATGAAACGTGTCATTGGTGGAGGTCTGAATGTGAGGCTGGGACAGAGGGAGAGAACAAGG
CCTCCCAGCACTCTGATGTCAGAGTAGTAGGTTAAGGGTGGAAGGTTGACCTACTTGGAT
CTTGGCATGCACCCACCTAACCCACTTTCTCAAGAACAAGAACCTAGAATGAATATCCAA
GCACCTCGAGCTATGCAACCTCTGTTCTTGTATTTCTTATGATCTCTGATGGGTTCTTCT
CGAAAATGCCAAGTGGAAGACTTTGTGGCATGCTCCAGATTTAAATCCAGCTGAGGCTCC
CTTTGTTTTCAGTTCCATGTAACAATCTGGAAGGAAACTTCACGGACAGGAAGACTGCTG
GAGAAGAGAAGCGTGTTAGCCCATTTGAGGTCTGGGGAATCATGTAAAGGGTACCCAGAC
CTCACTTTTAGTTATTTACATCAATGAGTTCTTTCAGGGAACCAAACCCAGAATTCGGTG
CAAAAGCCAAACATCTTGGTGGGATTTGATAAATGCCTTGGGACCTGGAGTGCTGGGCTT
GTGCACAGGAAGAGCACCAGCCGCTGAGTCAGGATCCTGTCAGTTCCATGAGCTATTCCT
CTTTGGTTTGGCTTTTTGATATGATTAAAATTATTTTTTATTCCTTTTTCTACTGTGTCT
TAAACACCAATTCCTGATAGTCCAAGGAACCACCTTTCTCCCTTGATATATTTAACTCCG
TCTTTGGCCTGACAACAGTCTTCTGCCCATGTCTGGGAACACACGCCAGGAGGAATGTCT
GATACCCTCTGCATCAAGCGTAAGAAGGTCCCAAATCATAACCATTTTAAGAACAGATGA
CTCAGAAACCTCCAGAGGAATCTGTTTGCTTCCTGATTAGATCCAGTCAATGTTTTAAAG
GTATTGTCAGAGAAAAACAGAGGGTCTGTACTAGCCATGCAAGGAGTCGCTCTAGCTGGT
ACCCGTAAAAGTTGTGGGATTGTGACCCCCCATCCCAAGGGGATGCCAAAATTTCTCTCA
TTCTTTTGGTATAAACTTAACATTAGCCAGGGAGGTTCTGGCTAACGTTAAATGCTGCTA
TACAACTGCTTTGCAACAGTTGCTGGTATATTTAAATCATTAAATTTCAGCATTTACTAA
T
>gi|4557573|gb|NP_000392.1|EXT2 718 aa linear exostoses
(multiple) 2 [Homo sapiens].
MCASVKYNIRGPALIPRMKTKHRIYYITLFSIVLLGLIATGMFQFWPHSIESSNDWNVEK
RSIRDVPVVRLPADSPIPERGDLSCRMHTCFDVYRCGFNPKNKIKVYIYALKKYVDDFGV
SVSNTISREYNELLMAISDSDYYTDDINRACLFVPSIDVLNQNTLRIKETAQAMAQLSRW
DRGTNHLLFNMLPGGPPDYNTALDVPRDRALLAGGGFSTWTYRQGYDVSIPVYSPLSAEV
DLPEKGPGPRQYFLLSSQVGLHPEYREDLEALQVKHGESVLVLDKCTNLSEGVLSVRKRC
HKHQVFDYPQVLQEATFCVVLRGARLGQAVLSDVLQAGCVPVVIADSYILPFSEVLDWKR
ASVVVPEEKMSDVYSILQSIPQRQIEEMQRQARWFWEAYFQSIKAIALATLQIINDRIYP
YAAISYEEWNDPPAVKWGSVSNPLFLPLIPPQSQGFTAIVLTYDRVESLFRVITEVSKVP
SLSKLLVVWNNQNKNPPEDSLWPKIRVPLKVVRTAENKLSNRFFPYDEIETEAVLAIDDD
IIMLTSDELQFGYEVWREFPDRLVGYPGRLHLWDHEMNKWKYESEWTNEVSMVLTGAAFY
HKYFNYLYTYKMPGDIKNWVDAHMNCEDIANNFLVANVTGKAVIKVTPRKKFKCPECTAI
DGLSLDQTHMVERSECINKFASVFGTMPLKVVEHRADPVLYKDDFPEKLKSFPNIGSL
>gi|27597083|gb|NM_006838.2|METAP2 1908 bp mRNA Homo sapiens
methionyl aminopeptidase 2 (METAP2), mRNA.
CTCTGTCTCATTCCCTCGCGCTCTCTCGGGCAACATGGCGGGTGTGGAGGAGGTAGCGGC
CTCCGGGAGCCACCTGAATGGCGACCTGGATCCAGACGACAGGGAAGAAGGAGCTGCCTC
TACGGCTGAGGAAGCAGCCAAGAAAAAAAGACGAAAGAAGAAGAAGAGCAAAGGGCCTTC
TGCAGCAGGGGAACAGGAACCTGATAAAGAATCAGGAGCCTCAGTGGATGAAGTAGCAAG
ACAGTTGGAAAGATCAGCATTGGAAGATAAAGAAAGAGATGAAGATGATGAAGATGGAGA
TGGCGATGGAGATGGAGCAACTGGAAAGAAGAAGAAAAAGAAGAAGAAGAAGAGAGGACC
AAAAGTTCAAACAGACCCTCCCTCAGTTCCAATATGTGACCTGTATCCTAATGGTGTATT
TCCCAAAGGACAAGAATGCGAATACCCACCCACACAAGATGGGCGAACAGCTGCTTGGAG
AACTACAAGTGAAGAAAAGAAAGCATTAGATCAGGCAAGTGAAGAGATTTGGAATGATTT
TCGAGAAGCTGCAGAAGCACATCGACAAGTTAGAAAATACGTAATGAGCTGGATCAAGCC
TGGGATGACAATGATAGAAATCTGTGAAAAGTTGGAAGACTGTTCACGCAAGTTAATAAA
AGAGAATGGATTAAATGCAGGCCTGGCATTTCCTACTGGATGTTCTCTCAATAATTGTGC
TGCCCATTATACTCCCAATGCCGGTGACACAACAGTATTACAGTATGATGACATCTGTAA
AATAGACTTTGGAACACATATAAGTGGTAGGATTATTGACTGTGCTTTTACTGTCACTTT
TAATCCCAAATATGATACGTTATTAAAAGCTGTAAAAGATGCTACTAACACTGGAATAAA
GTGTGCTGGAATTGATGTTCGTCTGTGTGATGTTGGTGAGGCCATCCAAGAAGTTATGGA
GTCCTATGAAGTTGAAATAGATGGGAAGACATATCAAGTGAAACCAATCCGTAATCTAAA
TGGACATTCAATTGGGCAATATAGAATACATGCTGGAAAAACAGTGCCGATTGTGAAAGG
AGGGGAGGCAACAAGAATGGAGGAAGGAGAAGTATATGCAATTGAAACCTTTGGTAGTAC
AGGAAAAGGTGTTGTTCATGATGATATGGAATGTTCACATTACATGAAAAATTTTGATGT
TGGACATGTGCCAATAAGGCTTCCAAGAACAAAACACTTGTTAAATGTCATCAATGAAAA
CTTTGGAACCCTTGCCTTCTGCCGCAGATGGCTGGATCGCTTGGGAGAAAGTAAATACTT
GATGGCTCTGAAGAATCTGTGTGACTTGGGCATTGTAGATCCATATCCACCATTATGTGA
CATTAAAGGATCATATACAGCGCAATTTGAACATACCATCCTGTTGCGTCCAACATGTAA
AGAAGTTGTCAGCAGAGGAGATGACTATTAAACTTAGTCCAAAGCCACCTCAACACCTTT
ATTTTCTGAGCTTTGTTGGAAAACATGATACCAGAATTAATTTGCCACATGTTGTCTGTT
TTAACAGTGGACCCATGTAATACTTTTATCCATGTTTAAAAAGAAGGAATTTGGACAAAG
GCAAACCGTCTAATGTAATTAACCAACGAAAAAGCTTTCCGGACTTTTAAATGCTAACTG
TTTTTCCCCTTCCTGTCTAGGAAAATGCTATAAAGCTCAAATTAGTTAGGAATGACTTAT
ACGTTTTGTTTTGAATACCTAAGAGATACTTTTTGGATATTTATATTGCCATATTCTTAC
TTGAATGCTTTGAATGACTACATCCAGTTCTGCACCTATACCCTCTGGTGTTGCTTTTTA
ACCTTCCTGGAATCCATTTCTAAAAAATAAAGACATTTTCAGATCTGA
>gi|5803092|gb|NP_006829.1|METAP2 478 aa linear methionyl
aminopeptidase 2; methionine aminopeptidase; eIF-2-
associated p67 [Homo sapiens].
MAGVEEVAASGSHLNGDLDPDDREEGAASTAEEAAKKKRRKKKKSKGPSAAGEQEPDKES
GASVDEVARQLERSALEDKERDEDDEDGDGDGDGATGKKKKKKKKKRGPKVQTDPPSVPI
CDLYPNGVFPKGQECEYPPTQDGRTAAWRTTSEEKKALDQASEEIWNDFREAAEAHRQVR
KYVMSWIKPGMTMIEICEKLEDCSRKLIKENGLNAGLAFPTGCSLNNCAAHYTPNAGDTT
VLQYDDICKIDFGTHISGRIIDCAFTVTFNPKYDTLLKAVKDATNTGIKCAGIDVRLCDV
GEAIQEVMESYEVEIDGKTYQVKPIRNLNGHSIGQYRIHAGKTVPIVKGGEATRMEEGEV
YAIETFGSTGKGVVHDDMECSHYMKNFDVGHVPIRLPRTKHLLNVINENFGTLAFCRRWL
DRLGESKYLMALKNLCDLGIVDPYPPLCDIKGSYTAQFEHTILLRPTCKEVVSRGDDY
>gi|10864040|gb|NM_021230.1|MLL3 12689 bp mRNA Homo sapiens
myeloid/lymphoid or mixed-lineage leukemia3 (MLL3), mRNA.
AAAATTCCTTAGTTGCTGGCTTTGACCTTTTATGTTGCTGAGTTTTACACATCTATTTTC
TCAACTGCCATATCCTAGGGGGCTTGGAGTACCCATAATACAGTGAGCCCACCTTCCTGG
TCCCCAGACATTTCAGAAGGTCGGGAAATTTTTAAACCCAGGCAGCTTCCTGGCAGTGCC
ATTTGGAGCATCAAAGTGGGCCGTGGGTCTGGATTTCCAGGAAAGCGGAGACCTCGAGGT
GCAGGACTGTCGGGGCGAGGTGGCCGAGGCAGGTCAAAGCTGAAAAGTGGAATCGGAGCT
GTTGTATTACCTGGGGTGTCTACTGCAGATATTTCATCAAATAAGGATGATGAAGAAAAC
TCTATGCACAATACAGTTGTGTTGTTTTCTAGCAGTGACAAGTTCACTTTGAATCAGGAT
ATGTGTGTAGTTTGTGGCAGTTTTGGCCAAGGAGCAGAAGGAAGATTACTTGCCTGTTCT
CAGTGTGGTCAGTGTTACCATCCATACTGTGTCAGTATTAAGATCACTAAAGTGGTTCTT
AGCAAAGGTTGGAGGTGTCTTGAGTGCACTGTGTGTGAGGCCTGTGGGAAGGCAACTGAC
CCAGGAAGACTCCTGCTGTGTGATGACTGTGACATAAGTTATCACACCTACTGCCTAGAC
CCTCCATTGCAGACAGTTCCCAAAGGAGGCTGGAAGTGCAAATGGTGTGTTTGGTGCAGA
CACTGTGGAGCAACATCTGCAGGTCTAAGATGTGAATGGCAGAACAATTACACACAGTGC
GCTCCTTGTGCAAGCTTATCTTCCTGTCCAGTCTGCTATCGAAACTATAGAGAAGAAGAT
CTTATTCTGCAATGTAGACAATGTGATAGATGGATGCATGCAGTTTGTCAGAACTTAAAT
ACTGAGGAAGAAGTGGAAAATGTAGCAGACATTGGTTTTGATTGTAGCATGTGCAGACCC
TATATGCCTGCGTCTAATGTGCCTTCCTCAGACTGCTGTGAATCTTCACTTGTAGCACAA
ATTGTCACAAAAGTAAAAGAGCTAGACCCACCCAAGACTTATACCCAGGATGGTGTGTGT
TTGACTGAATCAGGGATGACTCAGTTACAGAGCCTCACAGTTACAGTTCCAAGAAGAAAA
CGGTCAAAACCAAAATTGAAATTGAAGATTATAAATCAGAATAGCGTGGCCGTCCTTCAG
ACCCCTCCAGACATCCAATCAGAGCATTCAAGGGATGGTGAAATGGATGATAGTCGAGAA
GGAGAACTTATGGATTGTGATGGAAAATCAGAATCTAGTCCTGAGCGGGAAGCTGTGGAT
GATGAAACTAAGGGAGTGGAAGGAACAGATGGTGTCAAAAAGAGAAAAAGGAAACCATAC
AGACCAGGTATTGGTGGATTTATGGTGCGGCAAAGAAGTCGAACTGGGCAAGGGAAAACC
AAAAGATCTGTGATCAGAAAAGATTCCTCAGGCTCTATTTCCGAGCAGTTACCTTGCAGA
GATGATGGCTGGAGTGAGCAGTTACCAGATACTTTAGTTGATGAATCTGTTTCTGTTACT
GAAAGCACTGAAAAAATAAAGAAGAGATACCGAAAAAGGAAAAATAAGCTTGAAGAAACT
TTCCCTGCCTATTTACAAGAAGCTTTCTTTGGAAAAGATCTTCTAGATACAAGTAGACAA
AGCAAGATAAGTTTAGATAATCTGTCAGAAGATGGAGCTCAGCTTTTATATAAAACAAAC
ATGAACACAGGTTTCTTGGATCCTTCCTTAGATCCACTACTTAGTTCATCCTCGGCTCCA
ACAAAATCTGGAACTCACGGTCCTGCTGATGACCCATTAGCTGATATTTCTGAAGTTTTA
AACACAGATGATGACATTCTTGGAATAATTTCAGATGATCTAGCAAAATCAGTTGATCAT
TCAGATATTGGTCCTGTCACTGATGATCCTTCCTCTTTGCCTCAGCCAAATGTCAATCAG
AGTTCACGACCATTAAGTGAAGAACAGCTAGATGGGATCCTCAGTCCTGAACTAGACAAA
ATGGTCACAGATGGAGCAATTCTTGGAAAATTATATAAAATTCCAGAGCTTGGCGGAAAA
GATGTTGAAGACTTATTTACAGCTGTACTTAGTCCTGCGAACACTCAGCCAACTCCATTG
CCACAGCCTCCCCCACCAACACAGCTGTTGCCAATACACAATCAGGATGCTTTTTCACGG
ATGCCTCTCATGAATGGCCTTATTGGATCCAGTCCTCATCTCCCACATAATTCTTTGCCA
CCTGGAAGCGGACTGGGAACTTTCTCTGCAATTGCACAATCCTCTTATCCTGATGCCAGG
GATAAAAATTCAGCCTTTAATCCAATGGCAAGTGATCCTAACAACTCTTGGACATCATCA
GCTCCCACTGTGGAAGGAGAAAATGACACAATGTCGAATGCCCAGAGAAGCACGCTTAAG
TGGGAGAAAGAGGAGGCTCTGGGTGAAATGGCAACTGTTGCCCCAGTTCTCTACACCAAT
ATTAATTTCCCCAACTTAAAGGAAGAATTCCCTGATTGGACTACTAGAGTGAAGCAAATT
GCCAAATTGTGGAGAAAAGCAAGCTCACAAGAAAGAGCACCATATGTGCAAAAAGCCAGA
GATAACAGAGCTGCTTTACGCATTAATAAAGTACAGATGTCAAATGATTCCATGAAAAGG
CAGCAACAGCAAGATAGCATTGATCCCAGCTCTCGTATTGATTCGGAGCTTTTTAAAGAT
CCTTTAAAGCAAAGAGAATCAGAACATGAACAGGAATGGAAATTTAGACAGCAAATGCGT
CAGAAAAGTAAGCAGCAAGCTAAAATTGAAGCCACACAGAAACTTGAACAGGTGAAAAAT
GAGCAGCAGCAGCAGCAACAACAGCAATTTGGTTCTCAGCATCTTCTGGTGCAGTCTGGT
TCAGATACACCAAGTAGTGGGATACAGAGTCCCTTGACACCTCAGCCTGGCAATGGAAAT
ATGTCTCCTGCACAGTCATTCCATAAAGAACTGTTTACAAAACAGCCACCCAGTACCCCT
ACGTCTACATCTTCAGATGATGTGTTTGTAAAGCCACAAGCTCCACCTCCTCCTCCAGCC
CCATCCCGGATTCCCATCCAGGATAGTCTTTCTCAGGCTCAGACTTCTCAGCCACCCTCA
CCGCAAGTGTTTTCACCTQGGTCCTCTAACTCACGACCACCATCTCCAATGGATCCATAT
GCAAAAATGGTTGGTACCCCTCGACCACCTCCTGTGGGCCATAGTTTTTCCAGAAGAAAT
TCTGCTGCACCAGTGGAAAACTGTACACCTTTATCATCGGTATCTAGGCCCCTTCAAATG
AATGAGACAACAGCAAATAGGCCATCCCCTGTCAGAGATTTATGTTCTTCTTCCACGACA
AATAATGACCCCTATGCAAAACCTCCAGACACACCTAGGCCTGTGATGACAGATCAATTT
CCCAAATCCTTGGGCCTATCCCGGTCTCCTGTAGTTTCAGAACAAACTGCAAAAGGCCCT
ATAGCAGCTGGAACCAGTGATCACTTTACTAAACCATCTCCTAGGGCAGATGTGTTTCAA
AGACAAAGGATACCTGACTCATATGCACGACCCTTGTTGACACCTGCACCTCTTGATAGT
GGTCCTGGACCTTTTAAGACTCCAATGCAACCTCCTCCATCCTCTCAGGATCCTTATGGA
TCAGTGTCACAGGCATCAAGGCGATTGTCTGTTGACCCTTATGAAAGGCCTGCTTTGACA
CCAAGACCTATAGATAATTTTTCTCATAATCAGTCAAATGATCCATATAGTCAGCCTCCC
CTTACCCCACATCCAGCAGTGAATGAATCTTTTGCCCATCCTTCAAGGGCTTTTTCCCAG
CCTGGAACCATATCAAGGCCAACATCTCAGGACCCATACTCCCAACCCCCAGGAACTCCA
CGACCTGTTGTAGATTCTTATTCCCAATCTTCAGGAACAGCTAGGTCCAATACAGACCCT
TACTCTCAACCTCCTGGAACTCCCCGGCCTACTACTGTTGACCCATATAGTCAGCAGCCC
CAAACCCCAAGACCATCTACACAAACTGACTTGTTTGTTACACCTGTAACAAATCAGAGG
CATTCTGATCCATATGCTCATCCTCCTGGAACACCAAGACCTGGAATTTCTGTCCCTTAC
TCTCAGCCACCAGCAACACCAAGGCCAAGGATTTCAGAGGGTTTTACTAGGTCCTCAATG
ACAAGACCAGTCCTCATGCCAAATCAGGATCCTTTCCTGCAAGCAGCACAAAACCGAGGA
CCAGCTTTACCTGGCCCGTTGGTAAGGCCACCTGATACATGTTCCCAGACACCTAGGCCC
CCTGGACCTGGTCTTTCAGACACATTTAGCCGTGTTTCCCCATCTGCTGCCCGTGATCCC
TATGATCAGTCTCCAATGACTCCAAGATCTCAGTCTGACTCTTTTGGAACAAGTCAAACT
GCCCATGATGTTGCTGATCAGCCAAGGCCTGGATCAGAGGGGAGCTTCTGTGCATCTTCA
AACTCTCCAATGCACTCCCAAGGCCAGCAGTTCTCTGGTGTCTCCCAACTTCCTGGACCT
GTGCCAACTTCAGGAGTAACTGATACACAGAATACTGTAAATATGGCCCAAGCAGATACA
GAGAAATTGAGACAGCGGCAGAAGTTACGTGAAATCATTCTCCAGCAGCAACAGCAGAAG
AAGATTGCAGGTCGACAGGAGAAGGGGTCACAGGACTCACCCGCAGTGCCTCATCCAGGG
CCTCTTCAACACTGGCAACCAGAGAATGTTAACCAGGCTTTCACCAGACCCCCACCTCCC
TATCCTGGGAACATTAGGTCTCCTGTTGCCCCTCCTTTAGGACCTAGATATGCTGTTTTC
CCAAAAGATCAGCGTGGACCCTATCCTCCTGATGTTGCTAGTATGGGGATGAGACCTCAT
GGATTTAGATTTGGATTTCCAGGAGGTAGTCATGGTACCATGCCGAGTCAAGAGCGCTTC
CTTGTGCCTCCTCAGCAAATACAGGGATCTGGAGTTTCTCCACAGCTAAGAAGATCAGTA
TCTGTAGATATGCCTAGGCCTTTAAATAACTCACAAATGAATAATCCAGTTGGACTTCCT
CAGCATTTTTCACCACAGAGCTTGCCAGTTCAGCAGCACAACATACTGGGCCAAGCATAT
ATTGAACTGAGACATAGGGCTCCTGACGGAAGGCAACGGCTGCCTTTCAGTGCTCCACCT
GGCAGCGTTGTAGAGGCATCTTCTAATCTGAGACATGGAAACTTCATTCCCCGGCCAGAC
TTTCCGGGCCCTAGACACACAGACCCCATGCGACGACCTCCCCAGGGTCTACCTAATCAG
CTACCTGTGCACCCAGATTTGGAACAAGTGCCACCATCTCAACAAGAGCAAGGTCATTCT
GTCCATTCATCTTCTATGGTCATGAGGACTCTGAACCATCCACTAGGTGGTGAATTTTCA
GAAGCTCCTTTGTCAACATCTGTACCGTCTGAAACAACGTCTGATAATTTACAGATAACC
ACCCAGCCTTCTGATGGTCTAGAGGAAAAACTTGATTCTGATGACCCTTCTGTGAAGGAA
CTGGATGTTAAAGACCTTGAGGGGGTTGAAGTCAAAGACTTAGATGATGAAGATCTTGAA
AACTTAAATTTAGATACAGAGGATGGCAAGGTAGTTGAATTGGATACTTTAGATAATTTG
GAAACTAATGATCCCAACCTGGATGACCTCTTAAGGTCAGGAGAGTTTGATATCATTGCA
TATACAGATCCAGAACTTGACATGGGAGATAAGAAAAGCATGTTTAATGAGGAACTAGAC
CTTCCAATTGATGATAAGTTAGATAATCAGTGTGTATCTGTTGAACCAAAAAAAAAGGAA
CAAGAAAACAAAACTCTGGTTCTCTCTGATAAACATTCACCACAGAAAAAATCCACTGTT
ACCAATGAGGTAAAAACGGAAGTACTGTCTCCAAATTCTAAGGTGGAATCCAAATGTGAA
ACTGAAAAAAATGATGAGAATAAAGATAATGTTGACACTCCTTGCTCACAGGCTTCTGCT
CACTCAGACCTAAATGATGGAGAAAAGACTTCTTTGCATCCTTGTGATCCAGATCTATTT
GAGAAAAGAACCAATCGAGAAACTGCTGGCCCCAGTGCAAATGTCATTCAGGCATCCACT
CAACTACCTGCTCAAGATGTAATAAACTCTTGTGGCATAACTGGATCAACTCCAGTTCTC
TCAAGTTTACTTGCTAATGAGAAATCTGATAATTCAGACATTAGGCCATCGGGGTCTCCA
CCACCACCAACTCTGCCGGCCTCCCCATCCAATCATGTGTCAAGTTTGCCTCCTTTCATA
GCACCGCCTGGCCGTGTTTTGGATAATGCCATGAATTCTAATGTGACAGTAGTCTCTAGG
GTAAACCATGTTTTTTCTCAGGGTGTGCAGGTAAACCCAGGGCTCATTCCAGGTCAATCA
ACAGTTAACCACAGTCTGGGGACAGGAAAACCTGCAACTCAAACTGGGCCTCAAACAAGT
CAGTCTGGTACCAGTAGCATGTCTGGACCCCAACAGCTAATGATTCCTCAAACATTAGCA
CAGCAGAATAGAGAGAGGCCCCTTCTTCTAGAAGAACAGCCTCTACTTCTACAGGATCTT
TTGGATCAAGAAAGGCAAGAACAGCAGCAGCAAAGACAGATGCAAGCCATGATTCGTCAG
CGATCAGAACCGTTCTTCCCTAATATTGATTTTGATGCAATTACAGATCCTATAATGAAA
GCCAAAATGGTGGCCCTTAAAGGTATAAATAAAGTGATGGCACAAACAAATCTGGGCATG
CCACCAATGGTGATGAGCAGGTTCCCTTTTATGGGCCAGGTGGTAACTGGAACACAGAAC
AGTGAAGGACAGAACCTTGGACCACAGGCCATTCCTCAGGATGGCAGTATAACACATCAG
ATTTCTAGGCCTAATCCTCCAAATTTTGGTCCAGGCTTTGTCAATGATTCACAGCGTAAG
CAGTATGAAGAGTGGCTCCAGGAGACCCAACAGCTGCTTCAAATGCAGCAGAAGTATCTT
GAAGAACAAATTGGTGCTCACAGAAAATCTAAGAAGGCCCTTTCAGCTAAACAACGTACT
GCCAAGAAAGCTGGGCGTGAATTTCCAGAGGAAGATGCAGAACAACTCAAGCATGTTACT
GAACAGCAAAGCATGGTTCAGAAACAGCTAGAACAGATTCGTAAACAACAGAAAGAACAT
GCTGAATTGATTGAAGATTATCGGATCAAACAGCAGCAGCAATGTGCAATGGCCCCACCT
ACCATGATGCCCAGTGTCCAGCCCCAGCCACCCCTAATTCCAGGTGCCACTCCACCCACC
ATGAGCCAACCCACCTTTCCCATGGTGCCACAGCAGCTTCAGCACCAGCAGCACACAACA
GTTATTTCTGGCCATACTAGCCCTGTTAGAATGCCCAGTTTACCTGGATGGCAACCCAAC
AGTGCTCCTGCCCACCTGCCCCTCAATCCTCCTAGAATTCAGCCCCCAATTGCCCAGTTA
CCAATAAAAACTTGTACACCAGCCCCAGGGACAGTCTCAAATGCAAATCCACAGAGTGGA
CCACCACCTCGGGTAGAATTTGATGACAACAATCCCTTTAGTGAAAGTTTTCAAGAACGG
GAACGTAAGGAACGTTTACGAGAACAGCAAGAGAGACAACGGATCCAACTCATGCAGGAG
GTAGATAGACAAAGAGCTTTGCAGCAGAGGATGGAAATGGAGCAGCATGGTATGGTGGGC
TCTGAGATAAGTAGTAGTAGGACATCTGTGTCCCAGATTCCCTTCTACAGTTCCGACTTA
CCTTGTGATTTTATGCAACCTCTAGGACCCCTTCAGCAGTCTCCACAACACCAACAGCAA
ATGGGGCAGGTTTTACAGCAGCAGAATATACAACAAGGATCAATTAATTCACCCTCCACC
CAAACTTTCATGCAGACTAATGAGCGAAGGCAGGTAGGCCCTCCTTCATTTGTTCCTGAT
TCACCATCAATCCCTGTTGGAAGCCCAAATTTTTCTTCTGTGAAGCAGGGACATGGAAAT
CTTTCTGGGACCAGCTTCCAGCAGTCCCCAGTGAGGCCTTCTTTTACACCTGCTTTACCA
GCAGCACCTCCAGTAGCTAATAGCAGTCTCCCATGTGGCCAAGATTCTACTATAACCCAT
GGACACAGTTATCCGGGATCAACCCAATCGCTCATTCAGTTGTATTCTGATATAATCCCA
GAGGAAAAAGGGAAAAAGAAAAGAACAAGAAAGAAGAAAAGAGATGATGATGCAGAATCC
ACCAAGGCTCCATCAACTCCCCATTCAGATATAACTGCCCCACCGACTCCAGGCATCTCA
GAAACTACCTCTACTCCTGCAGTGAGCACACCCAGTGAGCTTCCTCAACAAGCCGACCAA
GAGTCGGTGGAACCAGTCGGCCCATCCACTCCCAATATGGCAGCAGGCCAGCTATGTACA
GAATTAGAGAACAAACTGCCCAATAGTGATTTCTCACAAGCAACTCCAAATCAACAGACG
TATGCAAATTCAGAAGTAGACAAGCTCTCCATGGAAACCCCTGCCAAAACAGAAGAGATA
AAACTGGAAAAGGCTGAGACAGAGTCCTGCCCAGGCCAAGAGGAGCCTAAATTGGAGGAA
CAGAATGGTAGTAAGGTAGAAGGAAACGCTGTAGCCTGTCCTGTCTCCTCAGCACAGAGT
CCTCCCCATTCTGCTGGGGCCCCTGCTGCCAAAGGAGACTCAGGGAATGAACTTCTGAAA
CACTTGTTGAAAAATAAAAAGTCATCTTCTCTTTTGAATCAAAAACCTGAGGGCAGTATT
TGTTCAGAAGATGACTGTACAAAGGATAATAAACTAGTTGAGAAGCAGAACCCAGCTGAA
GGACTGCAAACTTTGGGGGCTCAAATGCAAGGTGGTTTTGGATGTGGCAACCAGTTGCCA
AAAACAGATGGAGGAAGTGAAACCAAGAAACAGCGAAGCAAACGGACTCAGAGGACGGGT
GAGAAAGCAGCACCTCGCTCAAAGAAAAGGAAAAAGGACGAAGAGGAGAAACAAGCTATG
TACTCTAGCACTGACACGTTTACCCACTTGAAACAGGTGAGGCAGCTCTCTCTGCTCCCT
CTAATGGAACCAATCATTGGAGTGAACTTTGCGCACTTTCTTCCTTATGGCAGTGGCCAA
TTTAATAGTGGGAATCGACTTCTAGGAACTTTTGGCAGTGCTACCCTGGAAGGGGTTTCG
GACTACTATTCTCAGTTGATCTACAAGCAGAATAATTTAAGTAATCCTCCAACACCCCCT
GCCTCTCTTCCTCCTACACCACCTCCTATGGCTTGTCAGAAGATGGCCAATGGTTTTGCA
ACAACTGAAGAACTTGCTGGAAAAGCCGGAGTGTTAGTGAGCCATGAAGTTACCAAAACT
CTAGGACCTAAACCATTTCAGCTGCCCTTCAGACCCCAGGACGACTTGTTGGCCCGAGCT
CTTGCTCAGGGCCCCAAGACAGTTGATGTGCCAGCCTCCCTCCCAACACCACCTCATAAC
AATCAGGAAGAATTAAGGATACAGGATCACTGTGGTGATCGAGATACTCCTGACAGTTTT
GTTCCCTCATCCTCTCCTGAGAGTGTGGTTGGGGTAGAAGTGAGCAGGTATCCAGATCTG
TCATTGGTCAAGGAGGAGCCTCCAGAACCGGTGCCGTCCCCCATCATTCCAATTCTTCCT
AGCACTGCTGGGAAAAGTTCAGAATCAAGAAGGAATGACATCAAAACTGAGCCAGGCACT
TTATATTTTGCGTCACCTTTTGGTCCTTCCCCAAATGGTCCCAGATCAGGTCTTATATCT
GTAGCAATTACTCTGCATCCTACAGCTGCTGAGAACATTAGCAGTGTTGTGGCTGCATTT
TCCGACCTTCTTCACGTCCGAATCCCTAACAGCTATGAGGTTAGCAGTGCTCCAGATGTC
CCATCCATGGGTTTGGTCAGTAGCCACAGAATCAACCCGGGTTTGGAGTATCGACAGCAT
TTACTTCTCCGTGGGCCTCCGCCAGGATCTGCAAACCCTCCCAGATTAGTGAGCTCTTAC
CGGCTGAAGCAGCCTAATGTACCATTTCCTCCAACAAGCAATGGTCTTTCTGGATATAAG
GATTCTAGTCATGGTATTGCAGAAAGCGCAGCACTCAGACCACAGTGGTGTTGTCATTGT
AAAGTGGTTATTCTTGGAAGTGGTGTGCGGAAATCTTTCAAAGATCTGACCCTTTTGAAC
AAGGATTCCCGAGAAAGCACCAAGAGGGTAGAGAAGGACATTGTCTTCTGTAGTAATAAC
TGCTTTATTCTTTATTCATCAACTGCACAAGCGAAAAACTCAGAAAACAAGGAATCCATT
CCTTCATTGCCACAATCACCTATGAGAGAAACGCCTTCCAAAGCATTTCATCAGTACAGC
AACAACATCTCCACTTTGGATGTGCACTGTCTCCCCCAGCTCCCAGAGAAAGCTTCTCCC
CCTGCCTCACCACCCATCGCCTTCCCTCCTGCTTTTGAAGCAGCCCAAGTCGAGGCCAAG
CCAGATGAGCTGAAGGTGACAGTCAAGCTGAAGCCTCGGCTAAGAGCTGTCCATGGTGGG
TTTGAAGATTGCAGGCCGCTCAATAAAAAATGGAGAGGAATGAAATGGAAGAAGTGGAGC
ATTCATATTGTAATCCCTAAGGGGACATTTAAACCACCTTGTGAGGATGAAATAGATGAA
TTTCTAAAGAAATTGGGCACTTCCCTTAAACCTGATCCTGTGCCCAAAGACTATCGGAAA
TGTTGCTTTTGTCATGAAGAAGGTGATGGATTGACAGATGGACCAGCAAGGCTACTCAAC
CTTGACTTGGATCTGTGGGTCCACTTGAACTGCGCTCTGTGGTCCACGGAGGTCTATGAG
ACTCAGGCTGGTGCCTTAATAAATGTGGAGCTAGCTCTGAGGAGAGGCCTACAAATGAAA
TGTGTCTTCTGTCACAAGACGGGTGCCACTAGTGGATGCCACAGATTTCGATGCACCAAC
ATTTATCACTTCACTTGCGCCATTAAAGCACAATGCATGTTTTTTAAGGACAAAACTATG
CTTTGCCCCATGCACAAACCAAAGGGAATTCATGAGCAAGAATTAAGTTACTTTGCAGTC
TTCAGGAGGGTCTATGTTCAGCGTGATGAGGTGCGACAGATTGCTAGCATCGTGCAACGA
GGAGAACGGGACCATACCTTTCGCGTGGGTAGCCTCATCTTCCACACAATTGGTCAGCTG
CTTCCACAGCAGATGCAAGCATTCCATTCTCCTAAAGCACTCTTCCCTGTGGGCTATGAA
GCCAGCCGGCTGTACTGGAGCACTCGCTATGCCAATAGGCGCTGCCGCTACCTGTGCTCC
ATTGAGGAGAAGGATGGGCGCCCAGTGTTTGTCATCAGGATTGTGGAACAAGGCCATGAA
GACCTGGTTCTAAGTGACATCTCACCTAAAGGTGTCTGGGATAAGATTTTGGAGCCTGTG
GCATGTGTGAGAAAAAAGTCTGAAATGCTCCAGCTTTTCCCAGCGTATTTAAAAGGAGAG
GATCTGTTTGGCCTGACCGTCTCTGCAGTGGCACGCATAGCGGAATCACTTCCTGGGGTT
GAGGCATGTGAAAATTATACCTTCCGATACGGCCGAAATCCTCTCATGGAACTTCCTCTT
GCCGTTAACCCCACAGGTTGTGCCCGTTCTGAACCTAAAATGAGTGCCCATGTCAAGAGG
CCTCACACCTTAAACAGCACCAGCACCTCAAAGTCATTTCAGAGCACAGTCACTGGAGAA
CTGAACGCACCTTATAGTAAACAGTTTGTTCACTCCAAGTCATCGCAGTACCGGAAGATG
AAAACTGAATGGAAATCCAATGTGTATCTGGCACGGTCTCGGATTCAGGGGCTGGGCCTG
TATGCTGCTCGAGACATTGAGAAACACACCATGGTCATTGAGTACATCGGGACTATCATT
CGAAACGAAGTAGCCAACAGGAAAGAGAAGCTTTATGAGTCTCAGAACCGTGGTGTGTAC
ATGTTCCGCATGGATAACGACCATGTGATTGACGCGACGCTCACAGGAGGGCCCGCAAGG
TATATCAACCATTCGTGTGCACCTAATTGTGTGGCTGAAGTGGTGACTTTTGAGAGAGGA
CACAAAATTATCATCAGCTCCAGTCGGAGAATCCAGAAAGGAGAAGAGCTCTGCTATGAC
TATAAGTTTGACTTTGAAGATGACCAGCACAAGATTCCGTGTCACTGTGGAGCTGTGAAC
TGCCGGAAGTGGATGAACTGAAATGCATTCCTTGCTAGCTCAGCGGGCGGCTTGTCCCTA
GGAAGAGGCGATTCAACACACCATTGGAATTTTGCAGACAGAAAGAGATTTTTGTTTTCT
GTTTTATGACTTTTTGAAAAAGCTTCTGGGAGTTCTGATTTCCTCAGTCCTTTAGGTTAA
AGCAGCGCCAGGAGGAAGCTGACAGAAGCAGCGTTCCTGAAGTGGCCGAGGTTAAACGGA
ATCACAGAATGGTCCAGCACTTTTGCTTT
>gi|10864041|gb|NP_067053.1|MLL3 4025 aa linear myeloid/
lynphoid or mixed-lineage leukemia 3; ALR-like protein
[Homo sapiens].
MRNTVVLFSSSDKFTLNQDMCVVCGSFGQGAEGRLLACSQCGQCYEPYCVSIKITKVVLS
KGWRCLECTVCEACGKATDPGRLLLCDDCDISYHTYCLDPPLQTVPKGGWKCKWCVWCRH
CGATSAGLRCEWQNNYTQCAPCASLSSCPVCYRNYREEDLILQCRQCDRWMHAVCQNLNT
EEEVENVADIGFDCSMCRPYMPASNVPSSDCCESSLVAQIVTKVKELDPPKTYTQDGVCL
TESGMTQLQSLTVTVPRRKRSKPKLKLKIINQNSVAVLQTPPDIQSEHSRDGEMDDSREG
ELMDCDGKSESSPEREAVDDETKGVEGTDGVKKRKRKPYRPGIGGFMVRQRSRTGQGKTK
RSVIRKDSSGSISEQLPCRDDGWSEQLPDTLVDESVSVTESTEKIKKRYRKRKNKLEETF
PAYLQEAFFGKDLLDTSRQSKISLDNLSEDGAQLLYKTNMNTGFLDPSLDPLLSSSSAPT
KSGTHGPADDPLADISEVLNTDDDILGIISDDLAKSVDHSDIGPVTDDPSSLPQPNVNQS
SRPLSEEQLDGILSPELDKMVTDGAILGKLYKIPELGGKDVEDLFTAVLSPANTQPTPLP
QPPPPTQLLPIHNQDAFSRMPLMNGLIGSSPHLPHNSLPPGSGLGTFSAIAQSSYPDARD
KNSAFNPMASDPNNSWTSSAPTVEGENDTMSNAQRSTLKWEKEEALGEMATVAPVLYTNI
NFPNLKEEFPDWTTRVKQIAKLWRKASSQERAPYVQKARDNRAALRINKVQMSNDSMKRQ
QQQDSIDPSSRIDSELFKDPLKQRESEHEQEWKFRQQMRQKSKQQAKIEATQKLEQVKNE
QQQQQQQQFGSQHLLVQSGSDTPSSGIQSPLTPQPGNGNMSPAQSFHKELFTKQPPSTPT
STSSDDVFVKPQAPPPPPAPSRIPIQDSLSQAQTSQPPSPQVFSPGSSNSRPPSPMDPYA
KMVGTPRPPPVGHSFSRRNSAAPVENCTPLSSVSRPLQMNETTANRPSPVRDLCSSSTTN
NDPYAKPPDTPRPVMTDQFPKSLGLSRSPVVSEQTAKGPIAAGTSDHFTKPSPRADVFQR
QRIPDSYARPLLTPAPLDSGPGPFKTPMQPPPSSQDPYGSVSQASRRLSVDPYERPALTP
RPIDNFSHNQSNDPYSQPPLTPHPAVNESFAHPSRAFSQPGTISRPTSQDPYSQPPGTPR
PVVDSYSQSSGTARSNTDPYSQPPGTPRPTTVDPYSQQPQTPRPSTQTDLFVTPVTNQRH
SDPYAHPPGTPRPGISVPYSQPPATPRPRISEGFTRSSMTRPVLMPNQDPFLQAAQNRGP
ALPGPLVRPPDTCSQTPRPPGPGLSDTFSRVSPSAARDPYDQSPMTPRSQSDSFGTSQTA
HDVADQPRPGSEGSFCASSNSPMHSQGQQFSGVSQLPGPVPTSGVTDTQNTVNMAQADTE
KLRQRQKLREIILQQQQQKKIAGRQEKGSQDSPAVPHPGPLQHWQPENVNQAFTRPPPPY
PGNIRSPVAPPLGPRYAVFPKDQRGPYPPDVASMGMRPHGFRFGFPGGSHGTMPSQERFL
VPPQQIQGSGVSPQLRRSVSVDMPRPLNNSQMNWPVGLPQHFSPQSLPVQQHNILGQAYI
ELRHRAPDGRQRLPFSAPPGSVVEASSNLRHGNFIPRPDFPGPRHTDPMRRPPQGLPNQL
PVHPDLEQVPPSQQEQGESVHSSSMVMRTLNHPLGGEFSEAPLSTSVPSETTSDNLQITT
QPSDGLEEKLDSDDPSVKELDVKDLEGVEVKDLDDEDLENLNLDTEDGKVVELDTLDNLE
TNDPNLDDLLRSGEFDIIAYTDPELDMGDKKSMFNEELDLPIDDKLDNQCVSVEPKKKEQ
ENKTLVLSDKHSPQKKSTVTNEVKTEVLSPNSKVESKCETEKNDENKDNVDTPCSQASAH
SDLNDGEKTSLHPCDPDLFEKRTNRETAGPSANVIQASTQLPAQDVINSCGITGSTPVLS
SLLANEKSDNSDIRPSGSPPPPTLPASPSNHVSSLPPFIAPPGRVLDNAMNSNVTVVSRV
NHVFSQGVQVNPGLIPGQSTVNHSLGTGKPATQTGPQTSQSGTSSMSGPQQLMIPQTLAQ
QNRERPLLLEEQPLLLQDLLDQERQEQQQQRQMQAMIRQRSEPFFPNIDFDAITDPIMKA
KMVALKGINKVMAQNNLGMPPMVMSRFPFMGQVVTGTQNSEGQNLGPQAIPQDGSITHQI
SRPNPPNFGPGFVNDSQRKQYEEWLQETQQLLQMQQKYLEEQIGAHRKSKKALSAKQRTA
KKAGREFPEEDAEQLKHVTEQQSMVQKQLEQIRKQQKEHAELIEDYRIKQQQQCAMAPPT
MMPSVQPQPPLIPGATPPTMSQPTFPMVPQQLQHQQHTTVISGHTSPVRMPSLPGWQPNS
APAHLPLNPPRIQPPIAQLPIKTCTPAPGTVSNANPQSGPPPRVEFDDNNPFSESFQERE
RKERLREQQERQRIQLMQEVDRQRALQQRMEMEQHGMVGSEISSSRTSVSQIPFYSSDLP
CDFMQPLGPLQQSPQHQQQMGQVLQQQNIQQGSINSPSTQTFMQTNERRQVGPPSFVPDS
PSIPVGSPNFSSVKQGHGNLSGTSFQQSPVRPSFTPALPAAPPVANSSLPCGQDSTITHG
HSYPGSTQSLIQLYSDIIPEEKGKKKRTRKKKRDDDAESTKAPSTPHSDITAPPTPGISE
TTSTPAVSTPSELPQQADQESVEPVGPSTPNMAAGQLCTELENKLPNSDFSQATPNQQTY
ANSEVDKLSMETPAKTEEIKLEKAETESCPGQEEPKLEEQNGSKVEGNAVACPVSSAQSP
PHSAGAPAAKGDSGNELLKHLLKNKKSSSLLNQKPEGSICSEDDCTKDNKLVEKQNPAEG
LQTLGAQMQGGFGCGNQLPKTDGGSETKKQRSKRTQRTGEKAAPRSKKRKKDEEEKQAMY
SSTDTFTHLKQVRQLSLLPLMEPIIGVNFAHFLPYGSGQFNSGNRLLGTFGSATLEGVSD
YYSQLIYKQNNLSNPPTPPASLPPTPPPMACQKMANGFATTEELAGKAGVLVSHEVTKTL
GPKPFQLPFRPQDDLLARALAQGPKTVDVPASLPTPPHNNQEELRIQDHCGDRDTPDSFV
PSSSPESVVGVEVSRYPDLSLVKEEPPEPVPSPIIPILPSTAGKSSESRRNDIKTEPGTL
YFASPFGPSPNGPRSGLISVAITLHPTAAENISSVVAAFSDLLHVRIPNSYEVSSAPDVP
SMGLVSSHRINPGLEYRQHLLLRGPPPGSANPPRLVSSYRLKQPNVPFPPTSNGLSGYKD
SSHGIAESAALRPQWCCHCKVVILGSGVRKSFKDLTLLNKDSRESTKRVEKDIVFCSNNC
FILYSSTAQAKNSENKESIPSLPQSPMRETPSKAFHQYSNNISTLDVHCLPQLPEKASPP
ASPPIAFPPAFEAAQVEAKPDELKVTVKLKPRLRAVHGGFEDCRPLNKKWRGMKWKKWSI
HIVIPKGTFKPPCEDEIDEFLKKLGTSLKPDPVPKDYRKCCFCHEEGDGLTDGPARLLNL
DLDLWVHLNCALWSTEVYETQAGALINVELALRRGLQMKCVFCHKTGATSGCHRFRCTNI
YHFTCAIKAQCMFFKDKTMLCPMHKPKGIHEQELSYFAVFRRVYVQRDEVRQIASIVQRG
ERDHTFRVGSLIFHTIGQLLPQQMQAFHSPKALFPVGYEASRLYWSTRYANRRCRYLCSI
EEKDGRPVFVIRIVEQGHEDLVLSDISPKGVWDKILEPVACVRKKSEMLQLFPAYLKGED
LFGLTVSAVARIAESLPGVEACENYTFRYGRNPLMELPLAVNPTGCARSEPKMSAHVKRP
HTLNSTSTSKSFQSTVTGELNAPYSKQFVHSKSSQYRKMKTEWKSNVYLARSRIQGLGLY
AARDIEKHTMVIEYIGTIIRNEVANRKEKLYESQNRGVYMFRMDNDHVIDATLTGGPARY
INHSCAPNCVAEVVTFERGHKIIISSSRRIQKGEELCYDYKFDFEDDQHKIPCHCGAVNC
RKWMN
>gi|21359851|gb|NM_000966.2|RARG 2663 bp mRNA Homo sapiens
retinoic acid receptor, gamma (RARG), mRNA.
GGCACGAGGCAGTGGGCAGGCCAGGCAGGGCGGGTACGGAGCCTCCCAGGCTGGGGCAGT
GGGCATGGGCAGGGGCTGTGGCTGAAGACCTCGCCCGCCCACTGCAGACTCCAGGGGACT
CTCACACCGCAGCTGCCATGGCCACCAATAAGGAGCGACTCTTTGCGGCTGGTGCCCTGG
GGCCTGGATCTGGCTACCCAGGGGCAGGTTTCCCCTTCGCCTTCCCAGGGGCACTCAGGG
GGTCTCCGCCTTTCGAGATGCTGAGCCCTAGCTTCCGGGGCCTGGGCCAGCCTGACCTCC
CCAAGGAGATGGCCTCTCTGTCGGTGGAGACACAGAGCACCAGCTCAGAGGAGATGGTGC
CCAGCTCGCCCTCGCCCCCTCCGCCTCCTCGGGTCTACAAGCCATGCTTCGTGTGCAATG
ACAAGTCCTCTGGCTACCACTATGGGGTCAGCTCTTGTGAAGGCTGCAAGGGCTTCTTTC
GCCGAAGCATCCAGAAGAACATGGTGTACACGTGTCACCGCGACAAAAACTGTATCATCA
ACAAGGTGACCAGGAATCGCTGCCAGTACTGCCGGCTACAGAAGTGCTTCGAAGTGGGCA
TGTCCAAGGAAGCTGTGCGAAATGACCGGAACAAGAAGAAGAAAGAGGTGAAGGAAGAAG
GGTCACCTGACAGCTATGAGCTGAGCCCTCAGTTAGAAGAGCTCATCACCAAGGTCAGCA
AAGCCCATCAGGAGACTTTCCCCTCGCTCTGCCAGCTGGGCAAGTATACCACGAACTCCA
GTGCAGACCACCGCGTGCAGCTGGATCTGGGGCTGTGGGACAAGTTCAGTGAGCTGGCTA
CCAAGTGCATCATCAAGATCGTGGAGTTTGCCAAGCGGTTGCCTGGCTTTACAGGGCTCA
GCATTGCTGACCAGATCACTCTGCTCAAAGCTGCCTGCCTAGATATCCTGATGCTGCGTA
TCTGCACAAGGTACACCCCAGAGCAGGACACCATGACCTTCTCCGACGGGCTGACCCTGA
ACCGGACCCAGATGCACAATGCCGGCTTCGGGCCCCTCACAGACCTTGTCTTTGCCTTTG
CTGGGCAGCTCCTGCCCCTGGAGATGGATGACACCGAGACAGGGCTGCTCAGCGCCATCT
GCCTCATCTGCGGAGACCGCATGGACCTGGAGGAGCCCGAAAAAGTGGACAAGCTGCAGG
AGCCACTGCTGGAAGCCCTGAGGCTGTACGCCCGGCGCCGGCGGCCCAGCCAGCCCTACA
TGTTCCCAAGGATGCTAATGAAAATCACCGACCTCCGGGGCATCAGCACTAAGGGAGCTG
AAAGGGCCATTACTCTGAAGATGGAGATTCCAGGCCCGATGCCTCCCTTAATCCGAGAGA
TGCTGGAGAACCCTGAAATGTTTGAGGATGACTCCTCGCAGCCTGGTCCCCACCCCAATG
CCTCTAGCGAGGATGAGGTTCCTGGGGGCCAGGGCAAAGGGGGCCTGAAGTCCCCAGCCT
GACCAGGGCCCCTGACCTCCCCGCTGTGGGGGTTGGGGCTTCAGGCAGCAGACTGACCAT
CTCCCAGACCGCCAGTGACTGGGGGAGGACCTGCTCTGCCCTCTCCCCACCCCTTCCAAT
GAGCTCCTTGTTTTTGCCAAAGTTTCTAGGGGTGCCTCTGTGTTCATCCCCTTCCTGATC
TAACCGGCTCCCTCGCCAGTCCCGGGGGCCTGCCCTGCTCCCACCAGGAGAGAGGGCAAA
GGGATGAGCCTGGGTTTGGACTCTAAAATCTCAGCACTGCCCCATGGGTCCTAGACTTCC
CAGGGCAAGAGGAAGACCCTGCCATTCCACAGCCCCTTCCTCTGCCAGGTGCTTGGCTCT
CTGAGAGCAAACAGGAACACTAGAGACCAAAAAGGGGACAAAGGAGAAGGGCTGAGCCCA
CCTTCTTGCTCCTACCCTTGGTGCCTAATGCTGTGTGATGCACCTGCAGGGTGTGTGCTA
GCCTCTGTGCCCCGTCCTTGTGCCAGGTCAAGGTGGGGGCAGGCTGGGCCCTGCATTTCT
GGGGCAGGAACAGAGGGTGAAAGGGACAGATAGATGCAGGTCCATTCTGCACCTCTTGGC
TCGGGTGCAGAGTTCACCCTGTGCCCTCCGTTATAAGTCCCTCCCCCAGCCCTGTCATGT
GCCTTGGGCTCCTCCTGCCCTCCATCTCAGCCATTGGGGCAGGGACCCTCCTACACTACA
GAGGGGCCAGGGGATCCCTCTCTCCCTAGTGCCTTCCACCCTTTACTCCCCAGAGCAGCT
TGGCCCAGGGAGGGGGGATGCTGCTTAGCTGATCCCGCCCTGACCCAGAGGAAGCCTCTA
TTTATTTATTAGCTTTTGTTTACACCGTGGAATTGACCCCTTCCTCCAGGGGTCTTGGGT
GGGGGAGCCCAGGGCCCCTGTGACCCCTCCTTTCTTCCTCCAATCCCCAGTTTGTATTTA
GCTGCCAAATAAGATTCCCATTGGCTCCCTGTGTTCTCTTGGGGGGTCAGGGTGCTGTCC
CCTCCCCTCTGTTTACATCTCCCCTCTACCCCGCTGTATCGCATATTGCTGAGTTTTCTA
TTTTTGCAAAATAAAGTGATGGAAACTCAAAAAAAAAAAAAAAAAAAAAAAAAAAAAAAA
AAAAAAAAAAAAAAAAAAAAAAA
>gi|4506423|gb|NP_000957.1|RARG 454 aa linear retinoic acid
receptor, gamma; Retinoic acid receptor, gamma polypeptide
[Homo sapiens].
MATNKERLFAAGALGPGSGYPGAGFPFAFPGALRGSPPFEMLSPSFRGLGQPDLPKEMAS
LSVETQSTSSEEMVPSSPSPPPPPRVYKPCFVCNDKSSGYHYGVSSCEGCKGFFRRSIQK
NMVYTCHRDKNCIINKVTRNRCQYCRLQKCFEVGMSKEAVRNDRNKKKKEVKEEGSPSDY
ELSPQLEELITKVSKAHQETFPSLCQLGKYTTNSSADHRVQLDLGLWDKFSELATKCIIK
IVEFAKRLPGFTGLSIADQITLLKAACLDILMLRICTRYTPEQDTMTFSDGLTLNRTQMH
NAGFGPLTDLVFAFAGQLLPLEMDDTETGLLSAICLICGDRMDLEEPEKVDKLQEPLLEA
LRLYARRRRPSQPYMFPRMLMKITDLRGISTKGAERAITLKMEIPGPMPPLIREMLENPE
MFEDDSSQPGPHPNASSEDEVPGGQGKGGLKSPA
>gi|14670376|gb|NM_015318.1|P114-RHO-GEF 5113 bp mRNA Homo
sapiens Rho-specific guanine nucleotide exchange factor p114
(P114-RHO-GEF), mRNA.
GCTGGCGGAGAGCGGCCTGCGGGCGATCGGGCCGAGCCTCGCTCAAGGAGCACCCCCGGG
GCACCCTCCTGTCCGATGGCAGCCCGGCCCTGTCCAGGAATGTCGGTATGACGGTCTCTC
AGAAAGGGGGTCCCCAGCCAACACCGAGCCCGGCTGGCCCTGGGACGCAACTCGGACCAA
TCACAGGAGAGATGGATGAAGCCGATTCTGCGTTTTTAAAATTTAAGCAGACAGCTGATG
ACTCTCTGTCCCTTACATCTCCAAACACCGAGTCCATTTTTGTAGAAGATCCCTACACCG
CCTCGCTGAGGAGTGAGATTGAGTCAGACGGCCACGAGTTTGAAGCTGAGTCCTGGAGCC
TCGCCGTGGATGCAGCCTACGCCAAGAAGCAAAAGAGGGAGGTGGTGAAAAGACAAGATG
TCCTTTATGAGCTGATGCAGACAGAGGTGCACCACGTGCGGACGCTCAAGATCATGCTGA
AGGTGTACTCCAGGGCCCTGCAGGAGGAGCTGCAGTTCAGCAGCAAGGCCATTGGCCGCC
TCTTCCCATGCGCTGACGACCTGCTGGAGACGCACAGCCACTTCCTCGCTCGGCTCAAGG
AGCGCCGCCAGGAGTCCCTGGAGGAGGGCAGTGACCGGAATTATGTCATCCAGAAAATCG
GCGACCTCCTGGTTCAGCAGTTTTCAGGTGAAAATGGGGAGAGAATGAAAGAAAAGTACG
GTGTGTTTTGTAGTGGCCACAATGAAGCTGTTAGTCATTACAAGTTGCTGCTTCAGCAAA
ACAAGAAATTTCAAAACTTGATCAAGAAAATTGGCAACTTCTCCATCGTGCGGCGGCTTG
GCGTGCAGGAGTGCATTCTCCTGGTTACACAACGCATAACCAAATACCCAGTGCTGGTGG
AGCGCATCATCCAGAACACGGAAGCTGGCACTGAGGACTATGAAGACCTGACCCAGGCCT
TGAACCTCATCAAAGATATCATCTCACAAGTGGACGCCAAGGTCAGTGAGTGTGAGAAGG
GCCAGCGCCTCAGGGAGATCGCAGGGAAGATGGACCTGAAGTCTTCCAGCAAACTCAAGA
ACGGGCTCACCTTCCGCAAGGAAGACATGCTTCAGCGGCAGCTCCACCTGGAGGGCATGC
TATGCTGGAAGACCACATCAGGGCGCTTGAAAGATATCCTGGCTATCCTGCTGACCGACG
TACTTTTGCTGCTACAAGAAAAAGATCAGAAATACGTCTTTGCTTCTGTGGACTCAAAGC
CACCCGTCATCTCGTTACAAAAGCTCATCGTGAGGGAAGTGGCCAACGAGGAGAAAGCGA
TGTTTCTGATCAGCGCCTCCTTGCAAGGGCCGGAGATGTATGAAATCTACACGAGCTCCA
AAGAGGACAGGAACGCCTGGATGGCCCACATCCAAAGGGCTGTGGAGAGCTGCCCTGACG
AGGAGGAGGGGCCCTTCAGCCTGCCCGAAGAGGAAAGGAAGGTGGTCGAGGCCCGCGCCA
CGAGACTCCGGGACTTTCAAGAGCGGTTGAGCATGAAAGACCAGCTGATCGCACAGAGCC
TCCTAGAGAAACAGCAGATCTACCTGGAGATGGCCGAGATGGGCGGCCTCGAAGACCTGC
CCCAGCCCCGAGGCCTATTCCGTGGAGGGGACCCATCCGAGACCCTGCAGGGGGAGCTAA
TTCTCAAGTCGGCCATGAGCGAGATCGAGGGCATCCAGAGCCTGATCTGCAGGCGGCTGG
GCAGCGCCAACGGCCAGGCGGAAGACGGAGGCAGCTCCACAGGCCCGCCCAGGAGGGCTG
AGACCTTCGCGGGCTACGACTGCACAAACAGCCCCACCAAGAATGGCAGTTTCAAGAAGA
AAGTCAGCAGCACTGACCCCAGGCCCCGAGACTGGCGAGGCCCCCCAAACAGCCCGGACT
TGAAGCTCAGTGACAGTGACATTCCTGGGAGCTCTGAGGAATCGCCGCAGGTGGTGGAGG
CGCCAGGCACGGAATCCGATCCCCGTCTGCCCACCGTCCTGGAGTCGGAGCTTGTCCAGC
GGATCCAGACACTGTCCCAGCTGCTCCTGAACCTTCAGGCGGTAATCGCCCACCAGGACA
GCTATGTGGAGACGCAGCGGGCTGCCATCCAGGAGCGGGAGAAGCAGTTCCGGCTGCAGT
CGACGCGTGGGAACCTGCTGCTGGAGCAGGAGCGGCAACGCAACTTCGAGAAGCAGCGGG
AGGAGCGCGCGGCCCTGGAGAAGCTGCAGAGCCAGCTGCGGCACGAGCAGCAGCGCTGGG
AGCGCGAGCGCCAGTGGCAGCACCAGGAGCTGGAGCGTGCGGGCGCGCGGCTGCAGGAGC
GCGAGGGCGAGGCGCGGCAGCTACGCGAGCGGCTGGAGCAGGAGCGGGCCGAGCTGGAGC
GCCAGCGCCAGGCCTACCAGCACGACCTGGAGCGGCTGCGCGAGGCCCAGCGTGCCGTGG
AGCGCGAGCGGGAGCGCCTGGAGCTGCTGCGCCGCCTCAAGAAGCAGAACACCGCGCCAG
GCGCGCTGCCGCCCGACACACTGGCCGAGGCCCAGCCCCCAAGCCACCCTCCCAGCTTCA
ACGGGGAAGGGCTGGAGGGCCCTCGGGTGAGCATGCTGCCATCCGGCGTGGGGCCAGAGT
ACGCAGAGCGCCCCGAGGTGGCTCGCCGGGACAGCGCCCCCACCGAGAGCCGGCTGGCCA
AGAGCGATGTGCCCATCCAGCTGCTCAGCGCCACCAACCAGTTCCAGAGGCAGGCGGCCG
TGCAGCAGCAGATCCCCACCJAGCTGGCGGCCTCCACCAAGGGTGGCAAGGACAAGGGCG
GCAAGAGCAGGGGCTCTCAGCGCTGGGAGAGCTCAGCGTCCTTCGACCTGAAGCAGCAGC
TGCTGCTCAACAAGCTCATGGGGAAAGATGAGAGCACCTCACGGAACCGCCGCTCGCTGA
GCCCTATCCTGCCCGGCAGACACAGTCCTGCGCCCCCACCAGACCCTGGCTTCCCCGCCC
CGAGCCCACCGCCAGCTGACAGCCCCTCCGAGGGCTTCTCTCTCAAGGCCGGGGGCACAG
CCCTCCTGCCCGGCCCCCCAGCTCCCTCGCCACTGCCGGCCACACCACTCAGCGCCAAGG
AGGACGCCAGCAAAGAAGACGTCATCTTCTTCTAAAAGGGCCGTGACTCAAGGAAAGTTT
TTAATGGAAAGTTGAGCCAGAACTAAACCAGGGAGCTGTCTGAAATCATAGCACCCCATC
CGGGTGGCGGGGAGATCAACTCCGAGCTGTTTTTCCGAGGCAGTGAGGAACGGTGCCGGC
TCTGCACGGAGCTGAGGACAGGACAGACCTTGCTTTGAGAAGGAGCTGCCGGCCGGGGCC
ACGCTCCACAGCCGCCGCGCGACAGTGGAGCCAAGGGTTAGGGCACCAGGAGGGGCCAGG
TGGCGTCGGCAGCATCTGTCCCCAGAATCAGGCAGAATCCACTTCCCAAACAGAGCCCCA
CGCAGGTTCACCATGAACCTCAGGGTCAGGGAATGAGCCAGGCACGGGGGCATGGGCAGA
GAGGGCCACGGGGCAGGGCCCACTGAGGGAACATCAGTGGCCCTCCAGTCAGGTTCTGTG
GGTTTGGAAGCCCATCGTGAAAGGGGCTGACCTTTGCCCCTTTTTACTTGGCATTGGTTT
TGAAACCAGCTGTTTCCCAAACTCTGCTTCCCAAGGGCAACCGTTGCTGTTCACACGCTC
AGCCTGTCTGGGGGAGCGGGCCTCTAGCTTCAGCCAGGGCGGGTACACACCCTGGGCACA
GGGTCCTCAGCCCCCGGGAAATGAGCTCCCAGGGCTGGCGTCCCACCTTCCAGGTGGGGG
CTGGCACATCACAGACTGTCGAGAGCGCCATGTCCCAGGGCATGCAGAGGTTGCACCTAG
AGACGTTGCAGCAAGTGGACAAGTGGCCGCTGTGCGGGCCCCTCGCTTGTAGTGAGCTGT
TGCAGCTTACGGTCCGTTCCCTGGAGGGGTGGAGGAAGGAGGTGTTGGGCAGCATCAAAG
GTGCTGGGACATCCCAGGGTGGTGAGATCCATCCACGATCCAGCTCCGGTGGAGAAAGGG
CCCATGTCAAGCCTTGTTCTGCACCCCAAGCATTGGTGGTAGGACTGGGTCCTGGCTGAT
CGTCCTTGTTCCCAGTGGGGTACATGTGAGCCCCTGCCAGGGCCAAGTCCTTCTCCCGAA
CCCAGGGTCCTGGGAACTGCAGATCCCGGGGGGATTCAGCCCTTCTCCCACTGTGCTGGC
AGAGGCACTCCTGTGACGCTGAATACAGTGAACAGGGACATTCCCGCCACTCGGGGACAG
ATGGGCACAAGGGAGGGGAAACTCCATCAGGAAGTGCTCCCCTGGGCAGAGGCGCCCACT
GGGTGCTGTGGGCTCAGGAGGGGGCGGGGCAGGAGCTGGTGCCAACCGGGAACCAGAGCC
CCACAGCCATACAGCCCATTGGTGACAAGGTCCTGAGAACACAGTGGCCAGGTGTCCCCA
GGCTCCTGGCCCCTCCGACGACCTCAACTCTGCCCAGCCCGGTCCCTGGCCATCAGCGAC
GCTGTCCGCCCCCCGTCAGATCCCATGTGTGCCATGTTTATCATCAGTGTTTTGTATTTT
TGTACTGAGTATCGGAGCACTTTACAGAAGCTGACTGTACATTCCTGTTCTGTTGTGAAG
AGAACATTCCCAGACCCTGGCACCCTCCTGAGCCGGCGTGTGCCGGTCCAGCCCTCCGAG
ATGCCACAATTCCTTGGATGGGGGAGAAGTTCAAGGAATTTCTGCTCGGCCACGCGGTGG
GAACCCCGCGTCCCCGCCATGTGGCAGAGGGGTCTCAGTCGTGCTAGGCATCGGGCGGCA
GCGCCGACAGCCCTTCCCTCGCCAGTGCCCCTCGGCCACTCCTGGGTTGGAGCCCGATTT
TATTTGTAAAGTTGACAGTCGAGCAAATGTTCCTATTTTCGTGGGATCTGCACACGTCTT
TGTCAGTTGTGGTCATGATCTTAGTCACCTGCTAATTATTTTTACAATGATTACAACATT
TCCTCACTGCGGGATATTTCTGACCCGCTTTAGAACTTAAGACCTGATTCTAGCAATAAA
CGTGTCCGAGATG
>gi|14670377|gb|NP_056133.1|P114-RHO-GEF 1015 aa linear Rho-
specific guanine nucleotide exchange factor p114 [Homo
sapiens].
MTVSQKGGPQPTPSPAGPGTQLGPITGEMDEADSAFLKFKQTADDSLSLTSPNTESIFVE
DPYTASLRSEIESDGHEFEAESWSLAVDAAYAKKQKREVVKRQDVLYELMQTEVHHVRTL
KIMLKVYSRALQEELQFSSKAIGRLFPCADDLLETHSHFLARLKERRQESLEEGSDRNYV
IQKIGDLLVQQFSGENGERMKEKYGVFCSGHNEAVSHYKLLLQQNKKFQNLIKKIGNFSI
VRRLGVQECILLVTQRITKYPVLVERIIQNTEAGTEDYEDLTQALNLIKDIISQVDAKVS
ECEKGQRLREIAGKMDLKSSSKLKNGLTFRKEDMLQRQLHLEGMLCWKTTSGRLKDILAI
LLTDVLLLLQEKDQKYVFASVDSKPPVISLQKLIVREVANEEKAMFLISASLQGPEMYEI
YTSSKEDRNAWMAHIQRAVESCPDEEEGPFSLPEEERKVVEARATRLRDFQERLSMKDQL
IAQSLLEKQQIYLEMAEMGGLEDLPQPRGLFRGGDPSETLQGELILKSAMSEIEGIQSLI
CRRLGSANGQAEDGGSSTGPPRRAETFAGYDCTNSPTKNGSFKKKVSSTDPRPRDWRGPP
NSPDLKLSDSDIPGSSEESPQVVEAPGTESDPRLPTVLESELVQRIQTLSQLLLNLQAVI
AHQDSYVETQRAAIQEREKQFRLQSTRGNLLLEQERQRNFEKQREERAALEKLQSQLRHE
QQRWERERQWQHQELERAGARLQEREGEARQLRERLEQERAELERQRQAYQHDLERLREA
QRAVERERERLELLRRLKKQNTAPGALPPDTLAEAQPPSHPPSFNGEGLEGPRVSMLPSG
VGPEYAERPEVARRDSAPTESRLAKSDVPIQLLSATNQFQRQAAVQQQIPTKLAASTKGG
KDKGGKSRGSQRWESSASFDLKQQLLLNKLMGKDESTSRNRRSLSPILPGRHSPAPPPDP
GFPAPSPPPADSPSEGFSLKAGGTALLPGPPAPSPLPATPLSAKEDASKEDVIFF
>gi|23238259|gb|NM_005198.3|CHKL 1595 bp mRNA Homo sapiens
choline kinase-like (CHKL), transcript variant 1, mRNA.
CCCGGGCCGGGGCACGGAGAGAGCCGAGCGCCGCAGCCGTGAGCCGAATAGAGCCGGAGA
GACCCGAGTATGACCGGAGAAGCCCAGGCCGGCCGGAAGAGGAGCCGAGCGCGGCCGGAA
GGAACCGAGCCCGTCCGAAGGGAGCGGAGCGCAGCCTGGCCTGGGGCCCGGTCGAGCCCG
CGCCATGGCGGCCGAGGCGACAGCTGTGGCCGGAAGCGGGGCTGTTGGCGGCTGCCTGGC
CAAAGACGGCTTGCAGCAGTCTAAGTGCCCGGACACTACCCCAAAACGGCGGCGCGCCTC
GTCGCTGTCGCGTGACGCCGAGCGCCGAGCCTACCAATGGTGCCGGGAGTACTTGGGCGG
GGCCTGGCGCCGAGTGCAGCCCGAGGAGCTGAGGGTTTACCCCGTGAGCGGAGGCCTCAG
CAACCTGCTCTTCCGCTGCTCGCTCCCGGACCACCTGCCCAGCGTTGGCGAGGAGCCCCG
GGAGGTGCTTCTGCGGCTGTACGGAGCCATCTTGCAGGGCGTGGACTCCCTGGTGCTAGA
AAGCGTGATGTTCGCCATACTTGCGGAGCGGTCGCTGGGGCCCCAGCTGTACGGAGTCTT
CCCAGAGGGCCGGCTGGAACAGTACATCCCAAGTCGGCCATTGAAAACTCAAGAGCTTCG
AGAGCCAGTGTTGTCAGCAGCCATTGCCACGAAGATGGCGCAATTTCATGGCATGGAGAT
GCCTTTCACCAAGGAGCCCCACTGGCTGTTTGGGACCATGGAGCGGTACCTAAAACAGAT
CCAGGACCTGCCCCCAACTGGCCTCCCTGAGATGAACCTGCTGGAGATGTACAGCCTGAA
GGATGAGATGGGCAACCTCAGGAAGTTACTAGAGTCTACCCCATCGCCAGTCGTCTTCTG
CCACAATGACATCCAGGAAGGGAACATCTTGCTGCTCTCAGAGCCAGAAAATGCTGACAG
CCTCATGCTGGTGGACTTCGAGTACAGCAGTTATAACTATAGGGGCTTTGACATTGGGAA
CCATTTTTGTGAGTGGGTTTATGATTATACTCACGAGGAATGGCCTTTCTACAAAGCAAG
GCCCACAGACTACCCCACTCAAGAACAGCAGTTGCATTTTATTCGTCATTACCTGGCAGA
GGCAAAGAAAGGTGAGACCCTCTCCCAAGAGGAGCAGAGAAAACTGGAAGAAGATTTGCT
GGTAGAAGTCAGTCGGTATGCTCTGGCATCCCATTTCTTCTGGGGTCTGTGGTCCATCCT
CCAGGCATCCATGTCCACCATAGAATTTGGTTACTTGGACTATGCCCAGTCTCGGTTCCA
GTTCTACTTCCAGCAGAAGGGGCAGCTGACCAGTGTCGACTCCTCATCCTGACTCCACCC
TCCCACTCCTTGGATTTCTCCTGGAGCCTCCAGGGCAGGACCTTGGAGGGAGGAACAACG
AGCAGAAGGCCCTGGCGACTGGGCTGAGCCCCCAAGTGAAACTGAGGTTCAGGAGACCGG
CCTGTTCCTGAGTTTGAGTAGGTCCCCATGGCTGGCAGGCCAGAGCCCCGTGCTGTGTAT
GTAACACAATAAACAAGCTTCTTCTTCCCACCCTG
>gi|6978649|gb|NP_005189.2|CHKL 395 aa linear choline/
ethanolamine kinase isoform a [Homo sapiens].
MAAEATAVAGSGAVGGCLAKDGLQQSKCPDTTPKRRPASSLSRDAERRAYQWCREYLGGA
WRRVQPEELRVYPVSGGLSNLLFRCSLPDHLPSVGEEPREVLLRLYGAILQGVDSLVLES
VMFAILAERSLGPQLYGVFPEGRLEQYIPSRPLKTQELREPVLSAAIATKMAQFHGMEMP
FTKEPHWLFGTMERYLKQIQDLPPTGLPEMNLLEMYSLKDEMGNLRKLLESTPSPVVFCH
NDIQEGNILLLSEPENADSLMLVDFEYSSYNYRGFDIGNHFCEWVYDYTHEEWPFYKARP
TDYPTQEQQLHFIRHYLAEAKKGETLSQEEQRKLEEDLLVEVSRYALASHFFWGLWSILQ
ASMSTIEFGYLDYAQSRFQFYFQQKGQLTSVHSSS
>gi|4757755|gb|NM_004039.1|ANXA2 1362 bp mRNA Homo sapiens
annexin A2 (ANXA2), mRNA.
CATTTGGGGACGCTCTCAGCTCTCGGCGCACGGCCCAGCTTCCTTCAAAATGTCTACTGT
TCACGAAATCCTGTGCAAGCTCAGCTTGGAGGGTGATCACTCTACACCCCCAAGTGCATA
TGGGTCTGTCAAAGCCTATACTAACTTTGATGCTGAGCGGGATGCTTTGAACATTGAAAC
AGCCATCAAGACCAAAGGTGTGGATGAGGTCACCATTGTCAACATTTTGACCAACCGCAG
CAATGCACAGAGACAGGATATTGCCTTCGCCTACCAGAGAAGGACCAAAAAGGAACTTGC
ATCAGCACTGAAGTCAGCCTTATCTGGCCACCTGGAGACGGTGATTTTGGGCCTATTGAA
GACACCTGCTCAGTATGACGCTTCTGAGCTAAAAGCTTCCATGAAGGGGCTGGGAACCGA
CGAGGACTCTCTCATTGAGATCATCTGCTCCAGAACCAACCAGGAGCTGCAGGAAATTAA
CAGAGTCTACAAGGAAATGTACAAGACTGATCTGGAGAAGGACATTATTTCGGACACATC
TGGTGACTTCCGCAAGCTGATGGTTGCCCTGGCAAAGGGTAGAAGAGCAGAGGATGGCTC
TGTCATTGATTATGAACTGATTGACCAAGATGCTCGGGATCTCTATGACGCTGGAGTGAA
GAGGAAAGGAACTGATGTTCCCAAGTGGATCAGCATCATGACCGAGCGGAGCGTGCCCCA
CCTCCAGAAAGTATTTGATAGGTACAAGAGTTACAGCCCTTATGACATGTTGGAAAGCAT
CAGGAAAGAGGTTAAAGGAGACCTGGAAAATGCTTTCCTGAACCTGGTTCAGTGCATTCA
GAACAAGCCCCTGTATTTTGCTGATCGGCTGTATGACTCCATGAAGGGCAAGGGGACGCG
AGATAAGGTCCTGATCAGAATCATGGTCTCCCGCAGTGAAGTGGACATGTTGAAAATTAG
GTCTGAATTCAAGAGAAAGTACGGCAAGTCCCTGTACTATTATATCCAGCAAGACACTAA
GGGCGACTACCAGAAAGCGCTGCTGTACCTGTGTGGTGGAGATGACTGAAGCCCGACACG
GCCTGAGCGTCCAGAAATGGTGCTCACCATGCTTCCAGCTAACAGGTCTAGAAAACCAGC
TTGCGAATAACAGTCCCCGTGGCCATCCCTGTGAGGGTGACGTTAGCATTACCCCCAACC
TCATTTTAGTTGCCTAAGCATTGCCTGGCCTTCCTGTCTAGTCTCTCCTGTAAGCCAAAG
AAATGAACATTCCAAGGAGTTGGAAGTGAAGTCTATGATGTGAAACACTTTGCCTCCTGT
GTACTGTGTCATAAACAGATGAATAAACTGAATTTGTACTTT
>gi|4757756|gb|NP_004030.1|ANXA2 339 aa linear annexin A2;
annexin II; annexin II (lipocortin II); calpactin I, heavy
polypeptide (p36); lipocortin II; Annexin II (lipocortin I);
annexin II (lipocortin II; calpactin I, heavy polypeptide)
[Homo sapiens].
MSTVHEILCKLSLEGDHSTPPSAYGSVKAYTNFDAERDALNIETAIKTKGVDEVTIVNIL
TNRSNAQRQDIAFAYQRRTKKELASALKSALSGHLETVILGLLKTPAQYDASELKASMKG
LGTDEDSLIEIICSRTNQELQEINRVYKEMYKTDLEKDIISDTSGDFRKLMVALAKGRRA
EDGSVIDYELIDQDARDLYDAGVKRKGTDVPKWISIMTERSVPHLQKVFDRYKSYSPYDM
LESIRKEVKGDLENAFLNLVQCIQNKPLYFADRLYDSMKGKGTRDKVLIRIMVSRSEVDM
LKIRSEFKRKYGKSLYYYIQQDTKGDYQKALLYLCGGDD
>gi|27484939|gb|XM_084635.3|LOC143785 1982 bp mRNA Homo
sapiens similar to hypothetical protein XP_084635 [Homo
sapiens](LOC143785), mRNA.
TACTATCAGGGGGCAAGAGCCTTTCTCTCCAGCTACACACTCCATCTCCCGGGAGCAAGG
GGAAACTCCGAGAGGAGGGCAACAGAGCCAGCATCTTGCCAGGGCCCCGGAGGAGGGGTT
CCCCGCTACGCCTGTGCCGGAGGAGTTCCAGTCACCGAGCGAGGGGCGCAAGGGTGGGTG
CATCCTGCGCTGCGGCGGGCGCGCTACCCAGACGCTGGTGTGCAGAGCCACATGAAGCCT
GCTGGGGACTGGGGGCCAGGGAGCAGCAAGCCAGCTGGGACTGAGGCGGACGCTGTCTCA
GGGAGACGCTGACTCGCAAAGACACTCCCTTCCTTGTGCCTGGGTAAAAAGTCTCCTCCT
GGGGTCCCTGGCCATCCTGAATATCCAGAATGGTGTTTCTGAAGTTCTTCTGCATGAGTT
TCTTCTGCCACCTGTGTCAAGGCTACTTCGATGGCCCCCTCTACCCAGAGATGTCCAATG
GGACTCTGCACCACTACTTCGTGCCCGATGGGGACTATGAGGAGAACGATGACCCCGAGA
AGTGCCAGCTGCTCTTCAGGGTGAGTGACCACAGGCGCTGCTCCCAGGGGGAGGGGAGCC
AGGTTGGCAGCCTGCTGAGCCTCACCCTGCGGGAGGAGTTCACCGTGCTGGGCCGCCAGG
TGGAGGATGCTGGGCGCGTGCTGGAGGGCATCAGCAAAAGCATCTCCTACGACCTAGACG
GGGAAGAGAGCTATGGCAAGTACCTGCGGCGGGAGTCCCACCAGATCGGGGATGCCTACT
CCAACTCGGACAAATCCCTCACTGAGCTGGAGAGCAAGTTCAAGCAGGGCCAGGAACAGG
ACAGCCGGCAGGAGAGCAGGCTCAACGAGGACTTTCTGGGAATGCTGGTCCACACCAGGT
CCCTGCTGAAGGAGACACTGGACATCTCTGTGGGGCTCAGGGACAAATACGAGCTGCTGG
CCCTCACCATTAGGAGCCATGGGACCCGACTAGGTCGGCTGAAAAATGATTATCTTAAAG
TATAGGTGGAAGGATACAAATGCTAGAAAGAGGGAATCAAATCAGCCCCGTTTTGGAGGG
TGGGGGACAGAAGATGGGGCTACATTTCCCCCATACCTACTATTTTTTTATATCCCGATT
TGCACTTTGAGAATACATCTAAGGTCATCTTTCAAAAGAGAAAAATTGGACACTTGAGTG
ACTTTGTTTTTAGTTTTGTTTTTGTACATTATTTATGTGATTGTTATGGAATTGTCACCT
GGAAAGAACAATTTTAAGCAATGTCATTTCTAGATGGGTTTCTAATTCTGCAGAGACACC
CGTTTCAGCCACATCTAAAAGAGCACAGTTTATGTGGTGCGGAATTAAACTTCCCCATCC
TGCAGATTATGTGGAAATACCCAAAGATAATAGTGCATAGCTCCTTTCAGCCTCTAGCCT
TCACTCCTGGGCTCCAAAAGCTATCCCAGTTGCCTGTTTTTCAAATGAGGTTCAAGGTGC
TGCTTTGCATGCCTGCCAACCCATGGAAGTTGTTTCTTACTTCTTTTCTCTCTTATTTAT
TAACCATGGTCTGAGAGTTGTTTTTGTTCTATGTAACAGTATTGCCACAAAACTATAGGC
AAATCGTGTTTGCAGGGAGATTTCTGATGCCTCTGTGGGTGTGTGTAAGTTAAAGTGGCC
ACATTTAAGAAGGCCAAGCTTTGTAGTGGTTGCACAGTCACACTGATATGCTGATTTGCT
CTTTCTCATTGTATGTCTATGCTTTGTCATCAGTGCTATAGTAAATTACAAAGAAATAGG
TAGATTGTATGAACATACCCACAAATGCCTATGATTTAGGTTACCAATGTATTCTTTCTC
ATTTGGGGTTTTGCTTCTGTCTGTCTGTTTATTGGAAACTTGTACTTCAAGTAGGGGGAA
TCCTAATTCTAATAACTCCTTAGCTAAGTTTTATTATTCAGGCAATAAACATGTTTTCAT
GT
>gi|18578340|gb|XP_084635.1|LOC143785 211 aa linear similar
to hypothetical protein XP_084635 [Homo sapiens].
MVFLKFFCMSFFCHLCQGYFDGPLYPEMSNGTLHHYFVPDGDYEENDDPEKCQLLFRVSD
HRRCSQGEGSQVGSLLSLTLREEFTVLGRQVEDAGRVLEGISKSISYDLDGEESYGKYLR
RESHQIGDAYSNSDKSLTELESKFKQGQEQDSRQESRLNEDFLGMLVHTRSLLKETLDIS
VGLRDKYELLALTIRSHGTRLGRLKNDYLKV
>gi|4507464|gb|NM_003239.1|TGFB3 2574 bp mRNA Homo sapiens
transforming growth factor, beta 3 (TGFB3), mRNA.
CCTGTTTAGACACATGGACAACAATCCCAGCGCTACAAGGCACACAGTCCGCTTCTTCGT
CCTCAGGGTTGCCAGCGCTTCCTGGAAGTCCTGAAGCTCTCGCAGTGCAGTGAGTTCATG
CACCTTCTTGCCAAGCCTCAGTCTTTGGGATCTGGGGAGGCCGCCTGGTTTTCCTCCCTC
CTTCTGCACGTCTGCTGGGGTCTCTTCCTCTCCAGGCCTTGCCGTCCCCCTGGCCTCTCT
TCCCAGCTCACACATGAAGATGCACTTGCAAAGGGCTCTGGTGGTCCTGGCCCTGCTGAA
CTTTGCCACGGTCAGCCTCTCTCTGTCCACTTGCACCACCTTGGACTTCGGCCACATCAA
GAAGAAGAGGGTGGAAGCCATTAGGGGACAGATCTTGAGCAAGCTCAGGCTCACCAGCCC
CCCTGAGCCAACGGTGATGACCCACGTCCCCTATCAGGTCCTGGCCCTTTACAACAGCAC
CCGGGAGCTGCTGGAGGAGATGCATGGGGAGAGGGAGGAAGGCTGCACCCAGGAAAACAC
CGAGTCGGAATACTATGCCAAAGAAATCCATAAATTCGACATGATCCAGGGGCTGGCGGA
GCACAACGAACTGGCTGTCTGCCCTAAAGGAATTACCTCCAAGGTTTTCCGCTTCAATGT
GTCCTCAGTGGAGAAAAATAGAACCAACCTATTCCGAGCAGAATTCCGGGTCTTGCGGGT
GCCCAACCCCAGCTCTAAGCGGAATGAGCAGAGGATCGAGCTCTTCCAGATCCTTCGGCC
AGATGAGCACATTGCCAAACAGCGCTATATCGGTGGCAAGAATCTGCCCACACGGGGCAC
TGCCGAGTGGCTGTCCTTTGATGTCACTGACACTGTGCGTGAGTGGCTGTTGAGAAGAGA
GTCCAACTTAGGTCTAGAAATCAGCATTCACTGTCCATGTCACACCTTTCAGCCCAATGG
AGATATCCTGGAAAACATTCACGAGGTGATGGAAATCAAATTCAAAGGCGTGGACAATGA
GGATGACCATGGCCGTGGAGATCTGGGGCGCCTCAAGAAGCAGAAGGATCACCACAACCC
TCATCTAATCCTCATGATGATTCCCCCACACCGGCTCGACAACCCGGGCCAGGGGGGTCA
GAGGAAGAAGCGGGCTTTGGACACCAATTACTGCTTCCGCAACTTGGAGGAGAACTGCTG
TGTGCGCCCCCTCTACATTGACTTCCGACAGGATCTGGGCTGGAAGTGGGTCCATGAACC
TAAGGGCTACTATGCCAACTTCTGCTCAGGCCCTTGCCCATACCTCCGCAGTGCAGACAC
AACCCACAGCACGGTGCTGGGACTGTACAACACTCTGAACCCTGAAGCATCTGCCTCGCC
TTGCTGCGTGCCCCAGGACCTGGAGCCCCTGACCATCCTGTACTATGTTGGGAGGACCCC
CAAAGTGGAGCAGCTCTCCAACATGGTGGTGAAGTCTTGTAAATGTAGCTGAGACCCCAC
GTGCGACAGAGAGAGGGGAGAGAGAACCACCACTGCCTGACTGCCCGCTCCTCGGGAAAC
ACACAAGCAACAAACCTCACTGAGAGGCCTGGAGCCCACAACCTTCGGCTCCGGGCAAAT
GGCTGAGATGGAGGTTTCCTTTTGGAACATTTCTTTCTTGCTGGCTCTGAGAATCACGGT
GGTAAAGAAAGTGTGGGTTTGGTTAGAGGAAGGCTGAACTCTTCAGAACACACAGACTTT
CTGTGACGCAGACAGAGGGGATGGGGATAGAGGAAAGGGATGGTAAGTTGAGATGTTGTG
TGGCAATGGGATTTGGGCTACCCTAAAGGGAGAAGGAAGGGCAGAGAATGGCTGGGTCAG
GGCCAGACTGGAAGACACTTCAGATCTGAGGTTGGATTTGCTCATTGCTGTACCACATCT
GCTCTAGGGATCTGGATTATGTTATAcAGGCAAGCATTTTTTTTTTTTTAAAGACAGGTT
ACGAAGACAAAGTCCCAGAATTGTATCTCATACTGTCTGGGATTAAGGGCAAATCTATTA
CTTTTGCAAACTGTCCTCTACATCAATTAACATCGTGGGTCACTACAGGGAGAAAATCCA
GGTCATGCAGTTCCTGGCCCATCAACTGTATTGGGCCTTTTGGATATGCTGAACGCAGAA
GAAAGGGTGGAAATCAACCCTCTCCTGTCTGCCCTCTGGGTCCCTCCTCTCACCTCTCCC
TCGATCATATTTCCCCTTGGACACTTGGTTAGACGCCTTCCAGGTCAGGATGCACATTTC
TGGATTGTGGTTCCATGCAGCCTTGGGGCATTATGGGTCTTCCCCCACTTCCCCTCCAAG
ACCCTGTGTTCATTTGGTGTTCCTGGAAGCAGGTGCTACAACATGTGAGGCATTCGGGGA
AGCTGCACATGTGCCACACAGTGACTTGGCCCCAGACGCATAGACTGAGGTATAAAGACA
AGTATGAATATTACTCTCAAAATCTTTGTATAAATAAATATTTTTGGGGCATCCTGGATG
ATTTCATCTTCTGGAATATTGTTTCTAGAACAGTAAAAGCCTTATTCTAAGGTG
>gi|4507465|gb|NP_003230.1|TGFB3 412 aa linear transforming
growth factor, beta 3 [Homo sapiens].
MKMHLQRALVVLALLNFATVSLSLSTCTTLDFGHIKKKRVEAIRGQILSKLRLTSPPEPT
VMTHVPYQVLALYNSTRELLEEMHGEREEGCTQENTESEYYAKEIHKFDMIQGLAEHNEL
AVCPKGITSKVFRFNVSSVEKNRTNLFRAEFRVLRVPNPSSKRNEQRIELFQILRPDEHI
AKQRYIGGKNLPTRGTAEWLSFDVTDTVREWLLRRESNLGLEISIHCPCHTFQPNGDILE
NIHEVMEIKFKGVDNEDDHGRGDLGRLKKQKDHHNPHLILMMIPPHRLDNPGQGGQRKKR
ALDTNYCFRNLEENCCVRPLYIDFRQDLGWKWVHEPKGYYANFCSGPCPYLRSADTTHST
VLGLYNTLNPEASASPCCVPQDLEPLTILYYVGRTPKVEQLSNMVVKSCKCS
>gi|21735553|gb|NM_002419.2|MAP3K11 3603 bp mRNA Homo
sapiens mitogen-activated protein kinase kinase kinase 11
(MAP3K11), mRNA.
ACAAAGGGAGGAGGAAGAAGGGAGCGGGGTCGGAGCCGTCGGGGCCAAAGGAGACGGGGC
CAGGAACAGGCAGTCTCGGCCCAACTGCGGACGCTCCCTCCACCCCCTGCGCAAAAAGAC
CCAACCGGAGTTGAGGCGCTGCCCCTGAAGGCCCCACCTTACACTTGGCGGGGGCCGGAG
CCAGGCTCCCAGGACTGCTCCAGAACCGAGGGAAGCTCGGGTCCCTCCAAGCTAGCCATG
GTGAGGCGCCGGAGGCCCCGGGGCCCCACCCCCCCGGCCTGACCACACTGCCCTGGGTGC
CCTCCTCCAGAAGCCCGAGATGCGGGGGGCCGGGAGACAACACTCCTGGCTCCCCAGAGA
GGCGTGGGTCTGGGGCTGAGGGCCAGGGCCCGGATGCCCAGGTTCCGGGACTAGGGCCTT
GGCAGCCAGCGGGGGTGGGGACCACGGGCACCCAGAGAAGGTCCTCCACACATCCCAGCG
CCGGCTCCCGGCCATGGAGCCCTTGAAGAGCCTCTTCCTCAAGAGCCCTCTAGGGTCATG
GAATGGCAGTGGCAGCGGGGGTGGTGGGGGCGGTGGAGGAGGCCGGCCTGAGGGGTCTCC
AAAGGCAGCGGGTTATGCCAACCCGGTGTGGACAGCCCTGTTCGACTACGAGCCCAGTGG
GCAGGATGAGCTGGCCCTGAGGAAGGGTGACCGTGTGGAGGTGCTGTCCCGGGACGCAGC
CATCTCAGGAGACGAGGGCTGGTGGGCGGGCCAGGTGGGTGGCCAGGTGGGCATCTTCCC
GTCCAACTATGTGTCTCGGGGTGGTGGCCCGCCCCCCTGCGAGGTGGCCAGCTTCCAGGA
GCTGCGGCTGGAGGAGGTGATCGGCATTGGAGGCTTTGGCAAGGTGTACAGGGGCAGCTG
GCGAGGTGAGCTGGTGGCTGTGAAGGCAGCTCGCCAGGACCCCGATGAGGACATCAGTGT
GACAGCCGAGAGCGTTCGCCAGGAGGCCCGGCTCTTCGCCATGCTGGCACACCCCAACAT
CATTGCCCTCAAGGCTGTGTGCCTGGAGGAGCCCAACCTGTGCCTGGTGATGGAGTATGC
AGCCGGTGGGCCCCTCAGCCGAGCTCTGGCCGGGCGGCGCGTGCCTCCCCATGTGCTGGT
CAACTGGGCTGTGCAGATTGCCCGTGGGATGCACTACCTGCACTGCGAGGCCCTGGTGCC
CGTCATCCACCGTGATCTCAAGTCCAACAACATTTTGCTGCTGCAGCCCATTGAGAGTGA
CGACATGGAGCACAAGACCCTGAAGATCACCGACTTTGGCCTGGCCCGAGAGTGGCACAA
AACCACACAAATGAGTGCCGCGGGCACCTACGCCTGGATGGCTCCTGAGGTTATCAAGGC
CTCCACCTTCTCTAAGGGCAGTGACGTCTGGAGTTTTGGGGTGCTGCTGTGGGAACTGCT
GACCGGGGAGGTGCCATACCGTGGCATTGACTGCCTTGCTGTGGCCTATGGCGTAGCTGT
TAACAAGCTCACACTGCCCATCCCATCCACCTGCCCCGAGCCCTTCGCACAGCTTATGGC
CGACTGCTGGGCGCAGGACCCCCACCGCAGGCCCGACTTCGCCTCCATCCTGCAGCAGTT
GGAGGCGCTGGAGGCACAGGTCCTACGGGAAATGCCGCGGGACTCCTTCCATTCCATGCA
GGAAGGCTGGAAGCGCGAGATCCAGGGTCTCTTCGACGAGCTGCGAGCCAAGGAAAAGGA
ACTACTGAGCCGCGAGGAGGAGCTGACGCGAGCGGCGCGCGAGCAGCGGTCACAGGCGGA
GCAGCTGCGGCGGCGCGAGCACCTGCTGGCCCAGTGGGAGCTAGAGGTGTTCGAGCGCGA
GCTGACGCTGCTGCTGCAGCAGGTGGACCGCGAGCGACCGCACGTGCGCCGCCGCCGCGG
GACATTCAAGCGCAGCAAGCTCCGGGCGCGCGACGGCGGCGAGCGTATCAGCATGCCACT
CGACTTCAAGCACCGCATCACCGTGCAGGCCTCACCCGGCCTTGACCGGAGGAGAAACGT
CTTCGAGGTCGGGCCTGGGGATTCGCCCACCTTTCCCCGGTTCCGAGCCATCCAGTTGGA
GCCTGCAGAGCCAGGCCAGGCATGGGGCCGCCAGTCCCCCCGACGTCTGGAGGACTCAAG
CAATGGAGAGCGGCGAGCATGCTGGGCTTGGGGTCCCAGTTCCCCCAAGCCTGGGGAAGC
CCAGAATGGGAGGAGAAGGTCCCGCATGGACGAAGCCACATGGTACCTGGATTCAGATGA
CTCATCCCCCTTAGGATCTCCTTCCACACCCCCAGCACTCAATGGTAACCCCCCGCGGCC
TAGCCTGGAGCCCGAGGAGCCCAAGAGGCCTGTCCCCGCAGAGCGCGGTAGCAGCTCTGG
GACGCCCAAGCTGATCCAGCGGGCGCTGCTGCGCGGCACCGCCCTGCTCGCCTCGCTGGG
CCTTGGCCGCGACCTGCAGCCGCCGGGAGGCCCAGGACGCGAGCGCGGGGAGTCCCCGAC
AACACCCCCCACGCCAACGCCCGCGCCCTGCCCGACCGAGCCGCCCCCTTCCCCGCTCAT
CTGCTTCTCGCTCAAGACGCCCGACTCCCCGCCCACTCCTGCACCCCTGTTGCTGGACCT
GGGTATCCCTGTGGGCCAGCGGTCAGCCAAGAGCCCCCGACGTGAGGAGGAGCCCCGCGG
AGGCACTGTCTCACCCCCACCGGGGACATCACGCTCTGCTCCTGGCACCCCAGGCACCCC
ACGTTCACCACCCCTGGGCCTCATCAGCCGACCTCGGCCCTCGCCCCTTCGCAGCCGCAT
TGATCCCTGGAGCTTTGTGTCAGCTGGGCCACGGCCTTCTCCCCTGCCATCACCACAGCC
TGCACCCCGCCGAGCACCCTGGACCTTGTTCCCGGACTCAGACCCCTTCTGGGACTCCCC
ACCTGCCAACCCCTTCCAGGGGGGCCCCCAGGACTGCAGGGCACAGACCAAAGACATGGG
TGCCCAGGCCCCGTGGGTGCCGGAAGCGGGGCCTTGAGTGGGCCAGGCCACTCCCCCGAG
CTCCAGCTGCCTTAGGAGGAGTCACAGCATACACTGGAACAGGAGCTGGGTCAGCCTCTG
CAGCTGCCTCAGTTTCCCCAGGGACCCCACCCCCCTTTGGGGGTCAGGAACACTACACTG
CACAGGAAGCCTTCACACTGGAAGGGGGACCTGCGCCCCCACATCTGAAACCTGTAGGTC
CCCCCAGCTCACCTGCCCTACTGGGGCCCAACACTGTACCCAGCTGGTTGGGAGGACCAG
AGCCTGTCTCAGGGAATTGCCTGCTGGGGTGATGCAGGGAGGAGGGGAGGTGCAGGGAAG
AGGGGCCGGCCTCAGCTGTCACCAGCACTTTTGACCAAGTCCTGCTACTGCGGCCCCTGC
CCTAGGGCTTAGAGCATGGACCTCCTGCCCTGGGGGTCATCTGGGGCCAGGGCTCTCTGG
ATGCCTTCCTGCTGCCCCAGCCAGGGTTGGAGTCTTAGCCTCGGGATCCAGTGAAGCCAG
AAGCCAAATAAACTCAAAAGCTGTCTCCCCAAAAAAAAAAAAAAAAAAAAAAAAAAAAAA
AAA
>gi|4505195|gb|NP_002410.1|MAP3K11 847 aa linear mitogen-
activated protein kinase kinase kinase 11; mixed lineage
kinase 3; SH3 domain-containing proline-rich kinase;
protein-tyrosine kinase PTK1 [Homo sapiens].
MEPLKSLFLKSPLGSWNGSGSGGGGGGGGGRPEGSPKAAGYANPVWTALFDYEPSGQDEL
ALRKGDRVEVLSRDAAISGDEGWWAGQVGGQVGIFPSNYVSRGGGPPPCEVASFQELRLE
EVIGIGGFGKVYRGSWRGELVAVKAARQDPDEDISVTAESVRQEARLFAMLAHPNIIALK
AVCLEEPNLCLVMEYAAGGPLSRALAGRRVPPHVLVNWAVQIARGMHYLHCEALVPVIHR
DLKSNNILLLQPIESDDMEHKTLKITDFGLAREWHKTTQMSAAGTYAWMAPEVIKASTFS
KGSDVWSFGVLLWELLTGEVPYRGIDCLAVAYGVAVNKLTLPIPSTCPEPFAQLMADCWA
QDPHRRPDFASILQQLEALEAQVLREMPRDSFHSMQEGWKREIQGLFDELRAKEKELLSR
EEELTRAAREQRSQAEQLRRREHLLAQWELEVFERELTLLLQQVDRERPHVRRRRGTFKR
SKLRARDGGERISMPLDFKHRITVQASPGLDRRRNVFEVGPGDSPTFPRFPAIQLEPAEP
GQAWGRQSPRRLEDSSNGERRACWAWGPSSPKPGEAQNGRRRSRMDEATWYLDSDDSSPL
GSPSTPPALNGNPPRPSLEPEEPKRPVPAERGSSSGTPKLIQRALLRGTALLASLGLGRD
LQPPGGPGRERGESPTTPPTPTPAPCPTEPPPSPLICFSLKTPDSPPTPAPLLLDLGIPV
GQRSAKSPRREEEPRGGTVSPPPGTSRSAPGTPGTPRSPPLGLISRPRPSPLRSRIDPWS
FVSAGPRPSPLPSPQPAPRRAPWTLFPDSDPFWDSPPANPFQGGPQDCRAQTKDMGAQAP
WVPEAGP
>gi|4505784|gb|NM_000294.1|PHKG2 1571 bp mRNA Homo sapiens
phosphorylase kinase, gamma 2 (testis) (PHKG2), mRNA.
AAGGTGAGCGACTGCAGGCAAACCCGGCGACAGCGCAGCTCGCGTCGACCCTGGCTCCTC
TGCCTGCCCCCTCAGGCCCCCGCCTCCTTCAGGATGACGCTGGACGTGGGGCCGGAGGAT
GAGCTGCCCGACTGGGCCGCCGCCAAAGAGTTTTACCAGAAGTACGACCCTAAGGACGTC
ATCGGCAGAGGAGTGAGCTCTGTGGTCCGCCGTTGTGTTCATCGAGCTACTGGCCACGAG
TTTGCGGTGAAGATTATGGAAGTGACAGCTGAGCGGCTGAGTCCTGAGCAGCTGGAGGAG
GTGCGGGAAGCCACACGGCGAGAGACACACATCCTTCGCCAGGTCGCCGGCCACCCCCAC
ATCATCACCCTCATCGATTCCTACGAGTCTTCTAGCTTCATGTTCCTGGTGTTTGACCTG
ATGCGGAAGGGAGAGCTGTTTGACTATCTCACAGAGAAGGTGGCCCTCTCTGAAAAGGAA
ACCAGGTCCATCATGCGGTCTCTGCTGGAAGCAGTGAGCTTTCTCCATGCCAACAACATT
GTGCATCGAGATCTGAAGCCCGAGAATATTCTCCTAGATGACAATATGCAGATCCGACTT
TCAGATTTCGGGTTCTCCTGCCACTTGGAACCTGGCGAGAAGCTTCGAGAGTTGTGTGGG
ACCCCAGGGTATCTAGCGCCAGAGATCCTTAAATGCTCCATGGATGAAACCCACCCAGGC
TATGGCAAGGAGGTCGACCTCTGGGCCTGTGGGGTGATCTTGTTCACACTCCTGGCTGGC
TCGCCACCCTTCTGGCACCGGCGGCAGATCCTGATGTTACGCATGATCATGGAGGGCCAG
TACCAGTTCAGTTCCCCCGAGTGGGATGACCGTTCCAGCACTGTCAAAGACCTGATCTCC
AGGCTGCTGCAGGTGGATCCTGAGGCACGCCTGACAGCTGAGCAGGCCCTACAGCACCCC
TTCTTTGAGCGTTGTGAAGGCAGCCAACCCTGGAACCTCACCCCCCGCCAGCGGTTCCGG
GTGGCAGTGTGGACAGTGCTGGCTGCTGGACGAGTGGCCCTAAGCACCCATCGTGTACGG
CCACTGACCAAGAATGCACTGTTGAGGGACCCTTATGCGCTGCGGTCAGTGCGGCACCTC
ATCGACAACTGTGCCTTCCGGCTCTACGGGCACTGGGTAAAGAAAGGGGAGCAGCAGAAC
CGGGCGGCTCTCTTTCAGCACCGGCCCCCTGGGCCTTTTCCCATCATGGGCCCTGAAGAG
GAGGGAGACTCTGCTGCTATAACTGAGGATGAGGCCGTGCTTGTGCTGGGCTAGGACCTC
AACCCCAGGGATTCCCAGGAAGCAGAACTCTCCAGAAGAAGGGTTTTGATCATTCCAGCT
CCTCTGGGCTCTGGCCTCAGGCCCACTAATGATCCTGCTACCCTCTTGAAGACCAGCCCG
GTACCTCTCTCCCCACTGGCCAGGACTCTGAGATCAGAGCTGGGGTGGAAGGGAGCCATT
CTGAACGCCACGCCTGGCCCGGTCAGTGCTGCATGCACTGCATATGAAATAAAATCTGCT
ACACGCCAGGG
>gi|4505785|gb|NP_000285.1|PHKG2 406 aa linear phosphorylase
kinase, gamma 2 (testis); Phosphorylase kinase, gamma 2
(testis/liver) [Homo sapiens].
MTLDVGPEDELPDWAAAKEFYQKYDPKDVIGRGVSSVVRRCVHRATGHEFAVKIMEVTAE
RLSPEQLEEVREATRRETHILRQVAGHPHIITLIDSYESSSFMFLVFDLMRKGELFDYLT
EKVALSEKETRSIMRSLLEAVSFLHANNIVHRDLKPENILLDDNMQIRLSDFGFSCHLEP
GEKLRELCGTPGYLAPEILKCSMDETHPGYGKEVDLWACGVILFTLLAGSPPFWHRRQIL
MLRMIMEGQYQFSSPEWDDRSSTVKDLISRLLQVDPEARLTAEQALQHPFFERCEGSQPW
NLTPRQRFRVAVWTVLAAGRVALSTHRVRPLTKNALLRDPYALRSVRHLIDNCAFRLYGH
WVKKGEQQNRAALFQHRPPGPFPIMGPEEEGDSAAITEDEAVLVLG
>gi|5453789|gb|NM_006169.1|NNMT 952 bp mRNA Homo sapiens
nicotinamide N-methyltransferase (NNMT), mRNA.
TGAACTCTGGATGCTGTTAGCCTGAGACTCAGGAAGACAACTTCTGCAGGGTCACTCCCT
GGCTTCTGGAGGAAAGAGAAGGAGGGCAGTGCTCCAGTGGTACAGAAGTGAGACATAATG
GAATCAGGCTTCACCTCCAAGGACACCTATCTAAGCCATTTTAACCCTCGGGATTACCTA
GAAAAATATTACAAGTTTGGTTCTAGGCACTCTGCAGAAAGCCAGATTCTTAAGCACCTT
CTGAAAAATCTTTTCAAGATATTCTGCCTAGACGGTGTGAAGGGAGACCTGCTGATTGAC
ATCGGCTCTGGCCCCACTATCTATCAGCTCCTCTCTGCTTGTGAATCCTTTAAGGAGATC
GTCGTCACTGACTACTCAGACCAGAACCTGCAGGAGCTGGAGAAGTGGCTGAAGAAAGAG
CCAGAGGCCTTTGACTGGTCCCCAGTGGTGACCTATGTGTGTGATCTTGAAGGGAACAGA
GTCAAGGGTCCAGAGAAGGAGGAGAAGTTGAGACAGGCGGTCAAGCAGGTGCTGAAGTGT
GATGTGACTCAGAGCCAGCCACTGGGGGCCGTCCCCTTACCCCCGGCTGACTGCGTGCTC
AGCACACTGTGTCTGGATGCCGCCTGCCCAGACCTCCCCACCTACTGCAGGGCGCTCAGG
AACCTCGGCAGCCTACTGAAGCCAGGGGGCTTCCTGGTGATCATGGATGCGCTCAAGAGC
AGCTACTACATGATTGGTGAGCAGAAGTTCTCCAGCCTCCCCCTGGGCCGGGAGGCAGTA
GAGGCTGCTGTGAAAGAGGCTGGCTACACAATCGAATGGTTTGAGGTGATCTCGCAAAGT
TATTCTTCCACCATGGCCAACAACGAAGGACTTTTCTCCCTGGTGGCGAGGAAGCTGAGC
AGACCCCTGTGATGCCTGTGACCTCAATTAAAGCAATTCCTTTGACCTGTCA
>gi|5453790|gb|NP_006160.1|NNMT 264 aa linear nicotinamide
N-methyltransferase [Homo sapiens].
MESGFTSKDTYLSHFNPRDYLEKYYKFGSRHSAESQILKHLLKNLFKIFCLDGVKGDLLI
DIGSGPTIYQLLSACESFKEIVVTDYSDQNLQELEKWLKKEPEAFDWSPVVTYVCDLEGN
RVKGPEKEEKLRQAVKQVLKCDVTQSQPLGAVPLPPADCVLSTLCLDAACPDLPTYCRAL
RNLGSLLKPGGFLVIMDALKSSYYMIGEQKFSSLPLGREAVEAAVKEAGYTIEWFEVISQ
SYSSTMANNEGLFSLVARKLSRPL
>gi|4507668|gb|NM_003295.1|TPT1 830 bp mRNA Homo sapiens
tumor protein, translationally-controlled 1 (TPT1), mRNA.
CCCCCCCGAGCGCCGCTCCGGCTGCACCGCGCTCGCTCCGAGTTTCAGGCTCGTGCTAAG
CTAGCGCCGTCGTCGTCTCCCTTCAGTCGCCATCATGATTATCTACCGGGACCTCATCAG
CCACGATGAGATGTTCTCCGACATCTACAAGATCCGGGAGATCGCGGACGGGTTGTGCCT
GGAGGTGGAGGGGAAGATGGTCAGTAGGACAGAAGGTAACATTGATGACTCGCTCATTGG
TGGAAATGCCTCCGCTGAAGGCCCCGAGGGCGAAGGTACCGAAAGCACAGTAATCACTGG
TGTCGATATTGTCATGAACCATCACCTGCAGGAAACAAGTTTCACAAAAGAAGCCTACAA
GAAGTACATCAAAGATTACATGAAATCAATCAAAGGGAAACTTGAAGAACAGAGACCAGA
AAGAGTAAAACCTTTTATGACAGGGGCTGCAGAACAAATCAAGCACATCCTTGCTAATTT
CAAAAACTACCAGTTCTTTATTGGTGAAAACATGAATCCAGATGGCATGGTTGCTCTATT
GGACTACCGTGAGGATGGTGTGACCCCATATATGATTTTCTTTAAGGATGGTTTAGAAAT
GGAAAAATGTTAACAAATGTGGCAATTATTTTGGATCTATCACCTGTCATCATAACTGGC
TTCTGCTTGTCATCCACACAACACCAGGACTTAAGACAAATGGGACTGATGTCATCTTGA
GCTCTTCATTTATTTTGACTGTGATTTATTTGGAGTGGAGGCATTGTTTTTAAGAAAAAC
ATGTCATGTAGGTTGTCTAAAAATAAAATGCATTTAAACTCATTTGAGAG
>gi|4507669|gb|NP_003286.1|TPT1 172 aa linear tumor protein,
translationally-controlled 1; fortilin; histamine-releasing
factor [Homo sapiens].
MIIYRDLISHDEMFSDIYKIREIADGLCLEVEGKMVSRTEGNIDDSLIGGNASAEGPEGE
GTESTVITGVDIVMNHHLQETSFTKEAYKKYIKDYMKSIKGKLEEQRPERVKPFMTGAAE
QIKHILANFKNYQFFIGENMNPDGMVALLDYREDGVTPYMIFFKDGLEMEKC
>gi|27477073|gb|NM_018725.2|IL17BR 2077 bp mRNA Homo sapiens
interleukin 17B receptor (IL17BR), transcript variant 1,
mRNA.
AGCGCAGCGTGCGGGTGGCCTGGATCCCGCGCAGTGGCCCGGCGATGTCGCTCGTGCTGC
TAAGCCTGGCCGCGCTGTGCAGGAGCGCCGTACCCCGAGAGCCGACCGTTCAATGTGGCT
CTGAAACTGGGCCATCTCCAGAGTGGATGCTACAACATGATCTAATCCCCGGAGACTTGA
GGGACCTCCGAGTAGAACCTGTTACAACTAGTGTTGCAACAGGGGACTATTCAATTTTGA
TGAATGTAAGCTGGGTACTCCGGGCAGATGCCAGCATCCGCTTGTTGAAGGCCACCAAGA
TTTGTGTGACGGGCAAAAGCAACTTCCAGTCCTACAGCTGTGTGAGGTGCAATTACACAG
AGGCCTTCCAGACTCAGACCAGACCCTCTGGTGGTAAATGGACATTTTCCTACATCGGCT
TCCCTGTAGAGCTGAACACAGTCTATTTCATTGGGGCCCATAATATTCCTAATGCAAATA
TGAATGAAGATGGCCCTTCCATGTCTGTGAATTTCACCTCACCAGGCTGCCTAGACCACA
TAATGAAATATAAAAAAAAGTGTGTCAAGGCCGGAAGCCTGTGGGATCCGAACATCACTG
CTTGTAAGAAGAATGAGGAGACAGTAGAAGTGAACTTCACAACCACTCCCCTGGGAAACA
GATACATGGCTCTTATCCAACACAGCACTATCATCGGGTTTTCTCAGGTGTTTGAGCCAC
ACCAGAAGAAACAAACGCGAGCTTCAGTGGTGATTCCAGTGACTGGGGATAGTGAAGGTG
CTACGGTGCAGCTGACTCCATATTTTCCTACTTGTGGCAGCGACTGCATCCGACATAAAG
GAACAGTTGTGCTCTGCCCACAAACAGGCGTCCCTTTCCCTCTGGATAACAACAAAAGCA
AGCCGGGAGGCTGGCTGCCTCTCCTCCTGCTGTCTCTGCTGGTGGCCACATGGGTGCTGG
TGGCAGGGATCTATCTAATGTGGAGGCACGAAAGGATCAAGAAGACTTCCTTTTCTACCA
CCACACTACTGCCCCCCATTAAGGTTCTTGTGGTTTACCCATCTGAAATATGTTTCCATC
ACACAATTTGTTACTTCACTGAATTTCTTCAAAACCATTGCAGAAGTGAGGTCATCCTTG
AAAAGTGGCAGAAAAAGAAAATAGCAGAGATGGGTCCAGTGCAGTGGCTTGCCACTCAAA
AGAAGGCAGCAGACAAAGTCGTCTTCCTTCTTTCCAATGACGTCAACAGTGTGTGCGATG
GTACCTGTGGCAAGAGCGAGGGCAGTCCCAGTGAGAACTCTCAAGACCTCTTCCCCCTTG
CCTTTAACCTTTTCTGCAGTGATCTAAGAAGCCAGATTCATCTGCACAAATACGTGGTGG
TCTACTTTAGAGAGATTGATACAAAAGACGATTACAATGCTCTCAGTGTCTGCCCCAAGT
ACCACCTCATGAAGGATGCCACTGCTTTCTGTGCAGAACTTCTCCATGTCAAGCAGCAGG
TGTCAGCAGGAAAAAGATCACAAGCCTGCCACGATGGCTGCTGCTCCTTGTAGCCCACCC
ATGAGAAGCAAGAGACCTTAAAGGCTTCCTATCCCACCAATTACAGGGAAAAAACGTGTG
ATGATCCTGAAGCTTACTATGCAGCCTACAAACAGCCTTAGTAATTAAAACATTTTATAC
CAATAAAATTTTCAAATATTGCTAACTAATGTAGCATTAACTAACGATTGGAAACTACAT
TTACAACTTCAAAGCTGTTTTATACATAGAAATCAATTACAGTTTTAATTGAAAACTATA
ACCATTTTGATAATGCAACAATAAAGCATCTTCAGCCAAACATCTAGTCTTCCATAGACC
ATGCATTGCAGTGTACCCAGAACTGTTTAGCTAATATTCTATGTTTAATTAATGAATACT
AACTCTAAGAACCCCTCACTGATTCACTCAATAGCATCTTAAGTGAAAAACCTTCTATTA
CATGCAAAAAATCATTGTTTTTAAGATAACAAAAGTAGGGAATAAACAAGCTGAACCCAC
TTTTAAAAAAAAAAAAAAAAAAAAAAAAAAAAAAAAA
>gi|27477074|gb|NP_061195.2|IL17BR 502 aa linear IL-17B
receptor isoform 1 precursor; IL-17B receptor; interleukin
17 receptor homolog 1; interleukin 17 receptor homolog;
cytokine receptor CRL4 [Homo sapiens].
MSLVLLSLAALCRSAVPREPTVQCGSETGPSPEWMLQHDLIPGDLRDLRVEPVTTSVATG
DYSILMNVSWVLRADASIRLLKATKICVTGKSNFQSYSCVRCNYTEAFQTQTRPSGGKWT
FSYIGFPVELNTVYFIGAHNIPNANMNEDGPSMSVNFTSPGCLDHIMKYKKKCVKAGSLW
DPNITACKKNEETVEVNFTTTPLGNRYMALIQHSTIIGFSQVFEPHQKKQTRASVVIPVT
GDSEGATVQLTPYFPTCGSDCIRHKGTVVLCPQTGVPFPLDNNKSKPGGWLPLLLLSLLV
ATWVLVAGIYLMWRHERIKKTSFSTTTLLPPIKVLVVYPSEICFHHTICYFTEFLQNHCR
SEVILEKWQKKKIAEMGPVQWLATQKKAADKVVFLLSNDVNSVCDGTCGKSEGSPSENSQ
DLFPLAFNLFCSDLRSQIHLHKYVVVYFREIDTKDDYNALSVCPKYHLMKDATAFCAELL
HVKQQVSAGKRSQACHDGCCSL
>gi|14165275|gb|NM_032411.1|ECRG4 772 bp mRNA Homo sapiens
esophageal cancer related gene 4 protein (ECRG4), mRNA.
GGATAACCCGCGGCCGCGCCTGCCCGCTCGCACCCCTCTCCCGCGCCCGGTTCTCCCTCG
CAGCACCTCGAAGTGCGCCCCTCGCCCTCCTGCTCGCGCCCCGCCGCCATGGCTGCCTCC
CCCGCGCGGCCTGCTGTCCTGGCCCTGACCGGGCTGGCGCTGCTCCTGCTCCTGTGCTGG
GGCCCAGGTGGCATAAGTGGAAATAAACTCAAGCTGATGCTTCAAAAACGAGAAGCACCT
GTTCCAACTAAGACTAAAGTGGCCGTTGATGAGAATAAAGCCAAAGAATTCCTTGGCAGC
CTGAAGCGCCAGAAGCGGCAGCTGTGGGACCGGACTCGGCCCGAGGTGCAGCAGTGGTAC
CAGCAGTTTCTCTACATGGGCTTTGACGAAGCGAAATTTGAAGATGACATCACCTATTGG
CTTAACAGAGATCGAAATGGACATGAATACTATGGCGATTACTACCAACGTCACTATGAT
GAAGACTCTGCAATTGGTCCCCGGAGCCCCTACGGCTTTAGGCATGGAGCCAGCGTCAAC
TACGATGACTACTAACCATGACTTGCCACACGCTGTACAAGAAGCAAATAGCGATTCTCT
TCATGTATCTCCTAATGCCTTACACTACTTGGTTTCTGATTTGCTCTATTTCAGCAGATC
TTTCTACCTACTTTGGTGATCAAAAAAGAAGAGTTAAAACAACACATGTAAATGCCTTTT
GATATTTCATGGGAATGTTTAAAAATAGAAATAAAGCATTTTGTTAAAACGA
>gi|14165276|gb|NP_115787.1|ECRG4 148 aa linear esophageal
cancer related gene 4 protein [Homo sapiens].
MAASPARPAVLALTGLALLLLLCWGPGGISGNKLKLMLQKREAPVPTKTKVAVDENKAKE
FLGSLKRQKRQLWDRTRPEVQQWYQQFLYMGFDEAKFEDDITYWLNRDRNGHEYYGDYYQ
RHYDEDSAIGPRSPYGFRHGASVNYDDY
>gi|24025684|gb|NM_003017.2|SFRS3 1403 bp mRNA Homo sapiens
splicing factor, arginine/serine-rich 3 (SFRs3), mRNA.
CCGGGTGAGTGAGAGAGTTGGTTGGTGTTGGGCCGGAGGAAAGCGGGAAGACTCATCGGA
GCGTGTGGATTTGAGCCGCCGCATTTTTTAACCCTAGATCTCGAAATGCATCGTGATTCC
TGTCCATTGGACTGTAAGGTTTATGTAGGCAATCTTGGAAACAATGGCAACAAGACGGAA
TTGGAACGGGCTTTTGGCTACTATGGACCACTCCGAAGTGTGTGGGTTGCTAGAAACCCA
CCCGGCTTTGCTTTTGTTGAATTTGAAGATCCCCGAGATGCAGCTGATGCAGTCCGAGAG
CTAGATGGAAGAACACTATGTGGCTGCCGTGTAAGAGTGGAACTGTCGAATGGTGAAAAA
AGAAGTAGAAATCGTGGCCCACCTCCCTCTTGGGGTCGTCGCCCTCGAGATGATTATCGT
AGGAGGAGTCCTCCACCTCGTCGCAGATCTCCAAGAAGGAGAAGCTTCTCTCGCAGCCGG
AGCAGGTCCCTTTCTAGAGATAGGAGAAGAGAGAGATCGCTGTCTCGGGAGAGAAATCAC
AAGCCGTCCCGATCCTTCTCTAGGTCTCGTAGTCGATCTAGGTCAAATGAAAGGAAATAG
AAGACAGTTTGCAAGAGAAGTGGTGTACAGGAAATTACTTCATTTGACAGGAGTATGTAC
AGAAAATTCAAGTTTTGTTTGAGACTTCATAAGCTTGGTGCATTTTTAAGATGTTTTAGC
TGTTCAAATCTGTTTGTCTCTTGAAACAGTGACACAAAGGTGTAATTCTCTATGGTTTGA
AATGGATCATACGAGGCATGTAATACCAAGAATTGTTACTTTACAATGTTCCCTTAAGCA
AAATTGAATTTGCTTTGAACTTTTAGTTATGCACAGACTGATAATAAACCTCTAAACCTG
CCCAGCGGAAGTGTGTTTTTTTTTAAATTTAAATACAGAAACAACTGGCAAAAATTGAAC
TAAGATTTACTTTTTTTTCCATAGCTGGGATATAGGCTGCAGCTATAGTTGAACAAGCAG
TCTTTAAAAACTGCTGTGAAACACAGGCCATCAGGGAAAACGAAATGCTGCACTATTAAA
TTAGAGGTTTTTGAAAAATCCAACTCTCATCCTGGGCAGAGGTTGCCTAGTTGGTATAGA
ATGTTAAGTTTCAAGAAAGTTTACCTTTGCTTTAGGTCATAAGTTCCTTATTTGATTGCT
GTATATGGATACATGGCTGTTCGTGACATTCTTTATGTGCAAATTTGTGATTTCAAAAAT
GTCCTGCCAGTTTAAGGGTACATTGTAGAGCCGAACTTTGAGTTACTGTGCAAGATTTTT
TTTTCATGCTGTCATTTGTAATATGTTTTGTGAGAATCCTTGGGATTAAAGTTTTGGTTA
CAAATTGTTAAAAAAAAAAAAAA
>gi|4506901|gb|NP_003008.1|SFRS3 164 aa linear splicing
factor, arginine/serine-rich 3; splicing factor, arginine//
serine-rich, 20-kD [Homo sapiens].
MHRDSCPLDCKVYVGNLGNNGNKTELERAFGYYGPLRSVWVARNPPGFAFVEFEDPRDAA
DAVRELDGRTLCGCRVRVELSNGEKRSRNRGPPPSWGRRPRDDYRRRSPPPRRRSPRRRS
FSRSRSRSLSRDRRRERSLSRERNRKPSRSFSRSRSRSRSNERK
>gi|4759097|gb|NM_004593.1|SFRS10 1972 bp mRNA Homo sapiens
splicing factor, arginine/serine-rich 10 (transformer 2
homolog, Drosophila) (SFRS10), mRNA.
GAATTCGGCACGAGGGCGACCGGCGCGTCGTGCGGGGCTGCGGCGGAGCCTCCTTAAGGA
AGGTGCAAGAGGTTGGCAGCTTCGATTGAAGCACATCGACCGGCGACAGCAGCCAGGAGT
CATGAGCGACAGCGGCGAGCAGAACTACGGCGAGCGGGAATCCCGTTCTGCTTCCAGAAG
TGGAAGTGCTCACGGATCGGGGAAATCTGCAAGGCATACCCCTGCAAGGTCTCGCTCCAA
GGAAGATTCCAGGCGTTCCAGATCAAAGTCCAGGTCCCGATCTGAATCTAGGTCTAGATC
CAGAAGAAGCTCCCGAAGGCATTATACCCGGTCACGGTCTCGCTCCCGCTCCCATAGACG
ATCACGTAGCAGGTCTTACAGTCGAGATTATCGTAGACGGCACAGCCACAGCCATTCTCC
CATGTCTACTCGCAGGCGTCATGTTGGGAATCGGGCAAATCCTGATCCTAACTGTTGTCT
TGGAGTATTTGGGCTGAGCTTGTACACCACAGAAAGAGATCTAAGAGAAGTGTTCTCTAA
ATATGGTCCCATTGCCGATGTGTCTATTGTATATGACCAGCAGTCTAGGCGTTCAAGAGG
ATTTGCCTTTGTATATTTTGAAAATGTAGATGATGCCAAGGAAGCTAAAGAACGTGCCAA
TGGAATGGAGCTTGATGGGCGTAGGATCAGAGTTGATTTCTCTATAACAAAAAGACCACA
TACGCCAACACCAGGAATTTACATGGGGAGACCTACCTATGGCAGCTCTCGCCGTCGGGA
TTACTATGACAGAGGATATGATCGGGGCTATGATGATCGGGACTACTATAGCAGATCATA
CAGAGGAGGAGGTGGAGGAGGAGGAGGATGGAGAGCTGCCCAAGACAGGGATCAGATTTA
TAGAAGGCGGTCACCTTCTCCTTACTATAGTCGTGGAGGATACAGATCACGTTCCAGATC
TCGATCATACTCACCTCGTCGCTATTAAAGCATGAAGACTTTCTGAAACCTGCCCTAGAG
CTGGGATATTGTTTGTGGGCAATATTTTTTATTGTCTCTTGTTTAAAAAGTGAACAGTGC
CTAGTGAAGTTAGGTGACTTTTACACCTTTTACGATGACTACTTTTGGTGGAGTTGAAAT
GCTGTTTTCATTCTGCATTTGTGTAGTTTGGTGCTTTGTTCCAAGTTAAGTGTTTTCAGA
AAAGTATGTTTTGCATGTATTTTTTTACAGTCTAAATTTTGACTGCTGAGAAGTTTCTAT
TGTACAAAACTTCATTTAAAAGGTTTTTCTACTGAATCCAGGGTATTCTGAAGATCGAAG
CCTGTGTAAAATGCTACCAAATGGCAAAAAGCAACAATAAACAGTTTGATTTTTACTTTT
CTTTCTAACATATCAATGCTTAGCAGAACTATTCAGATTGTCAGTAGTAAATTTAAAGAC
AAATGCCCGTTTTCCTCCAGTCCATGAAACATACCATACTTATATACCTGCAACTAAGTG
TTTAAAATTATGCTCTGTAACTCTGTACTGCTAGTATTAGAACTAAAAATCTTAAAATAC
AGCCAGTGCTTAATGCTTATATCAATGTGGATTTGTCGGCTTTTATGTAATCTGTAATAT
GTATAGCAGGAAATACGAAGAGTTACACAGTGTATGCCTTAAAAGGCTGTTTCTTAAAGG
TGTTACAAGGGGATAATGGTATTTCAACTAGTTATCAGCAAGTGACAATACATTCCACCA
CAAATACACTCTTGTTCTTCTAGCTTTTAGACTATATGAAAAAACCGGGTGCTTCAAAGT
ACATGATAAGGGAACACTATACCTGTCATGGATGAACTGAAGACTTTGCCTGTTCATTTT
TTAAATATTATTTTCAGGTCCTTTGCTTACCAAAGGAGGCCCAATTTCACTCAAATGTTT
TGAGAACTGTGTTTAAATAAACGCAAATGAAAAGAAAAAAAAAAAAAAAAAA
>gi|4759098|gb|NP_004584.1|SFRS10 288 aa linear splicing
factor, arginine/serine-rich 10 (transformer 2 homolog,
Drosophila); splicing factor, arginine/serine-rich
(transformer 2 Drosophila homolog) 10 [Homo sapiens].
MSDSGEQNYGERESRSASRSGSAHGSGKSARHTPARSRSKEDSRRSRSKSRSRSESRSRS
RRSSRRHYTRSRSRSRSHRRSRSRSYSRDYRRRHSHSHSPMSTRRRHVGNRANPDPNCCL
GVFGLSLYTTERDLREVFSKYGPIADVSIVYDQQSRRSRGFAFVYFENVDDAKEAKERAN
GMELDGRRIRVDFSITKRPHTPTPGIYMGRPTYGSSRRRDYYDRGYDRGYDDRDYYSRSY
RGGGGGGGGWRAAQDRDQIYRRRSPSPYYSRGGYRSRSRSRSYSPRRY
>gi|5803206|gb|NM_006758.1|U2AF1 904 bp mRNA Homo sapiens
U2(RNU2) small nuclear RNA auxiliary factor 1 (U2AF1), mRNA.
GGAATTCCGTCGACGGCAGCGGCGGCGGCGGGTGGGAAATGGCGGAGTATCTGGCCTCCA
TCTTCGGCACCGAGAAAGACAAAGTCAACTGTTCATTTTATTTCAAAATTGGAGCATGTC
GTCATGGAGACAGGTGCTCTCGGTTGCACAATAAACCGACGTTTAGCCAGACCATTGCCC
TCTTGAACATTTACCGTAACCCTCAAAACTCTTCCCAGTCTGCTGACGGTTTGCGCTGTG
CCGTGAGCGATGTGGAGATGCAGGAACACTATGATGAGTTTTTTGAGGAGGTTTTTACAG
AAATGGAGGAGAAGTATGGGGAAGTAGAGGAGATGAACGTCTGTGACAACCTGGGAGACC
ACCTGGTGGGGAACGTGTACGTCAAGTTTCGCCGTGAGGAAGATGCGGAAAAGGCTGTGA
TTGACTTGAATAACCGTTGGTTTAATGGACAGCCGATCCACGCCGAGCTGTCACCCGTGA
CGGACTTCAGAGAAGCCTGCTGCCGTCAGTATGAGATGGGAGAATGCACACGAGGCGGCT
TCTGCAACTTCATGCATTTGAAGCCCATTTCCAGAGAGCTGCGGCGGGAGCTGTATGGCC
GCCGTCGCAAGAAGCATAGATCAAGATCCCGATCCCGGGAGCGTCGTTCTCGGTCTAGAG
ACCGTGGTCGTGGCGGTGGCGGTGGCGGTGGTGGAGGTGGCGGCGGACGGGAGCGTGACA
GGAGGCGGTCGAGAGATCGTGAAAGATCTGGGCGATTCTGAGCCATGCCATTTTTACCTT
ATGTCTGCTAGAAAGTGTTGTAGTTGATTGACCAAACCAGTTCATAAGGGGAATTTTTTA
AAAAACAACAAAAAAAAAACATACAAAGATGGGTTTCTGAATAAAAATTTGTAGTGATAA
CAGT
>gi|5803207|gb|NP_006749.1|U2AF1 240 aa linear U2 small
nuclear RNA auxiliary factor 1; U2 snRNP auxiliary factor
small subunit; splicing factor U2AF 35kDa subunit [Homo
sapiens].
MAEYLASIFGTEKDKVNCSFYFKIGACRHGDRCSRLHNKPTFSQTIALLNIYRNPQNSSQ
SADGLRCAVSDVEMQEHYDEFFEEVFTEMEEKYGEVEEMNVCDNLGDHLVGNVYVKFRRE
EDAEKAVIDLNNRWFNGQPIHAELSPVTDFREACCRQYEMGECTRGGFCNFMHLKPISRE
LRRELYGRRRKKHRSRSRSRERRSRSRDRGRGGGGGGGGGGGGRERDRRRSRDRERSGRF
>gi|23308726|gb|NM_003242.3|TGFBR2 2090 bp mRNA Homo sapiens
transforming growth factor, beta receptor II (70/80kDa)
(TGFBR2), mnRNA.
GTTGGCGAGGAGTTTCCTGTTTCCCCCGCAGCGCTGAGTTGAAGTTGAGTGAGTCACTCG
CGCGCACGGAGCGACGACACCCCCGCGCGTGCACCCGCTCGGGACAGGAGCCGGACTCCT
GTGCAGCTTCCCTCGGCCGCCGGGGGCCTCCCCGCGCCTCGCCGGCCTCCAGGCCCCTCC
TGGCTGGCGAGCGGGCGCCACATCTGGCCCGCACATCTGCGCTGCCGGCCCGGCGCGGGG
TCCGGAGAGGGCGCGGCGCGGAGCGCAGCCAGGGGTCCGGGAAGGCGCCGTCCGTGCGCT
GGGGGCTCGGTCTATGACGAGCAGCGGGGTCTGCCATGGGTCGGGGGCTGCTCAGGGGCC
TGTGGCCGCTGCACATCGTCCTGTGGACGCGTATCGCCAGCACGATCCCACCGCACGTTC
AGAAGTCGGTTAATAACGACATGATAGTCACTGACAACAACGGTGCAGTCAAGTTTCCAC
AACTGTGTAAATTTTGTGATGTGAGATTTTCCACCTGTGACAACCAGAAATCCTGCATGA
GCAACTGCAGCATCACCTCCATCTGTGAGAAGCCACAGGAAGTCTGTGTGGCTGTATGGA
GAAAGAATGACGAGAACATAACACTAGAGACAGTTTGCCATGACCCCAAGCTCCCCTACC
ATGACTTTATTCTGGAAGATGCTGCTTCTCCAAAGTGCATTATGAAGGAAAAAAAAAAGC
CTGGTGAGACTTTCTTCATGTGTTCCTGTAGCTCTGATGAGTGCAATGACAACATCATCT
TCTCAGAAGAATATAACACCAGCAATCCTGACTTGTTGCTAGTCATATTTCAAGTGACAG
GCATCAGCCTCCTGCCACCACTGGGAGTTGCCATATCTGTCATCATCATCTTCTACTGCT
ACCGCGTTAACCGGCAGCAGAAGCTGAGTTCAACCTGGGAAACCGGCAAGACGCGGAAGC
TCATGGAGTTCAGCGAGCACTGTGCCATCATCCTGGAAGATGACCGCTCTGACATCAGCT
CCACGTGTGCCAACAACATCAACCACAACACAGAGCTGCTGCCCATTGAGCTGGACACCC
TGGTGGGGAAAGGTCGCTTTGCTGAGGTCTATAAGGCCAAGCTGAAGCAGAACACTTCAG
AGCAGTTTGAGACAGTGGCAGTCAAGATCTTTCCCTATGAGGAGTATGCCTCTTGGAAGA
CAGAGAAGGACATCTTCTCAGACATCAATCTGAAGCATGAGAACATACTCCAGTTCCTGA
CGGCTGAGGAGCGGAAGACGGAGTTGGGGAAACAATACTGGCTGATCACCGCCTTCCACG
CCAAGGGCAACCTACAGGAGTACCTGACGCGGCATGTCATCAGCTGGGAGGACCTGCGCA
AGCTGGGCAGCTCCCTCGCCCGGGGGATTGCTCACCTCCACAGTGATCACACTCCATGTG
GGAGGCCCAAGATGCCCATCGTGCACAGGGACCTCAAGAGCTCCAATATCCTCGTGAAGA
ACGACCTAACCTGCTGCCTGTGTGACTTTGGGCTTTCCCTGCGTCTGGACCCTACTCTGT
CTGTGGATGACCTGGCTAACAGTGGGCAGGTGGGAACTGCAAGATACATGGCTCCAGAAG
TCCTAGAATCCAGGATGAATTTGGAGAATGCTGAGTCCTTCAAGCAGACCGATGTCTACT
CCATGGCTCTGGTGCTCTGGGAAATGACATCTCGCTGTAATGCAGTGGGAGAAGTAAAAG
ATTATGAGCCTCCATTTGGTTCCAAGGTGCGGGAGCACCCCTGTGTCGAAAGCATGAAGG
ACAACGTGTTGAGAGATCGAGGGCGACCAGAAATTCCCAGCTTCTGGCTCAACCACCAGG
GCATCCAGATGGTGTGTGAGACGTTGACTGAGTGCTGGGACCACGACCCAGAGGCCCGTC
TCACAGCCCAGTGTGTGGCAGAACGCTTCAGTGAGCTGGAGCATCTGGACAGGCTCTCGG
GGAGGAGCTGCTCGGAGGAGAAGATTCCTGAAGACGGCTCCCTAAACACTACCAAATAGC
TCTTATGGGGCAGGCTGGGCATGTCCAAAGAGGCTGCCCCTCTCACCAAA
>gi|23308727|gb|NP_003233.3|TGFBR2 567 aa linear trans-
forming growth factor, beta receptor II (70/80kDa); trans-
forming growth factor, beta receptor II (70-80kD) [Homo
sapiens].
MGRGLLRGLWPLHIVLWTRIASTIPPHVQKSVNNDMIVTDNNGAVKFPQLCKFCDVRFST
CDNQKSCMSNCSITSICEKPQEVCVAVWRKNDENITLETVCHDPKLPYHDFILEDAASPK
CIMKEKKKPGETFFMCSCSSDECNDNIIFSEEYNTSNPDLLLVIFQVTGISLLPPLGVAI
SVIIIFYCYRVNRQQKLSSTWETGKTRKLMEFSEHCAIILEDDRSDISSTCANNINHNTE
LLPIELDTLVGKGRFAEVYKAKLKQNTSEQFETVAVKTFPYEEYASWKTEKDIFSDINLK
HENILQFLTAEERKTELGKQYWLITAFHAKGNLQEYLTRHVISWEDLRKLGSSLARGIAH
LRSDHTPCGRPKMPIVHRDLKSSNILVKNDLTCCLCDFGLSLRLDPTLSVDDLANSGQVG
TARYMAPEVLESRMNLENAESFKQTDVYSMALVLWEMTSRCNAVGEVKDYEPPFGSKVRE
HPCVESMKDNVLRDRGRPEIPSFWLNHQGIQMVCETLTECWDHDPEARLTAQCVAERFSE
LEHLDRLSGRSCSEEKIPEDGSLNTTK
>gi|5174728|gb|NM_006022.1|TSC22 1725 bp mRNA Homo sapiens
transforming growth factor beta-stimulated protein TSC-22
(TSC22), mRNA.
CGCCTCTTCACGGCACTGGGATCCGCATCTGCCTGGGATCATCAAGCCCTAGAAGCTGGG
TTTCTTTAAATTAGGGCTGCCGTTTTCTGTTTCTCCCTGGGCTGCGGAAAGCCAGAAGAT
TTTATCTAGCTTATACAAGGCTGCTGGTGTTCCCTCTTTTTTTCCACGAGGGTGTTTTTG
GCTGGAATTGCATGAAATCCCAATGGTGTAGACCAGTGGCGATGGATCTAGGAGTTTACC
AACTGAGACATTTTTCAATTTCTTTCTTGTCATCCTTGCTGGGGACTGAAAACGCTTCTG
TGAGACTTGATAATAGCTCCTCTGGTGCAAGTGTGGTAGCTATTGACAACAAAATCGAGC
AAGCTATGGATCTAGTGAAAAGCCATTTGATGTATGCGGTCAGAGAAGAAGTGGAGGTCC
TCAAAGAGCAAATCAAAGAACTAATAGAGAAAAATTCCCAGCTGGAGCAGGAGAACAATC
TGCTGAAGACACTGGCCAGTCCTGAGCAGCTTGCCCAGTTTCAGGCCCAGCTGCAGACTG
GCTCCCCCCCTGCCACCACCCAGCCACAGGGCACCACACAGCCCCCCGCCCAGCCAGCAT
CGCAGGGCTCAGGACCAACCGCATAGCTGCCTATGCCCCCGCAGAACTGGCTGCTGCGTG
TGAACTGAACAGACGGAGAAGATGTGCTAGGGAGAATCTGCCTCCACAGTCACCCATTTC
ATTGCTCGCTGCGAAAGAGACGTGAGACTGACATATGCCATTATCTCTTTTCCAGTATTA
AACACTCATATGCTTATGGCTTGGAGAAATTTCTTAGTTGGGTGAATTAAAGGTTAATCC
GAGAATTAGCATGGATATACCGGGACCTCATGCAGCTTGGCAGATATCTGAGAAATGGTT
TAATTCATGCTCAGGAGCTGTGTGCCTTTCCATCCCTTCCGGCTCCCTACCCCTCACTTC
CAAGGGTTCTCTCTCCTGCTTGCGCTTAGTGTCCTACATGGGGTTGTGAAGCGATGGAGC
TCCTCACTGGACTCGCCTCTCTCCTCTCCTCCCCCCAGGAGGAACTTGAAAGGAGGGTAA
AAAGACTAAAATGAGGGGGAACAGAGTTCACTGTACAAATTTGACAACTGTCACCAAAAT
TCATAAAAAACAATAGTACTGTGCCTCTTTCTTCTCAAACAATGGATGACACAAAACTAT
GAGAGTGACAAAATGGTGACAGGTAGCTGGGACCTAGGCTATCTTACCATGAAGGTTGTT
TTGCTTATTGTATATTTGTGTATGTAGTGTAACTATTTTGTACAATAGAGGACTGTAACT
ACTATTTAGGTTGTACAGATTGAAATTTAGTTGTTTCATTGGCTGTCTGAGGAGGTGTGG
ACTTTTATATATAGATCTACATAAAAACTGCTACATGACAAAAACCACACCTAAACCCCT
TTTAAGAATTTGGCACAGTTACTCACTTTGTGTAATCTGAAATCTAGCTGCTGAATACGC
TGAAGTAAATCCTTGTTCACTGAAGTCTTTCAATTGAGCTGGTTGAATACTTTGAAAAAT
GCTCAGTTCTAACTAATGAAATGGATTTCCCAGTAGGGGTTTCTGCATATCACCTGTATA
GTAGTTATATGCATATGTTTCTGTGCATGTTCTCTACACAATTGTAAGGTGTCACTGTAT
TTAACTGTTGCACTTGTCAACTTTCAATAAAGCATATAAATGTTG
>gi|5174729|gb|NP_006013.1|TSC22 144 aa linear transforming
growth factor beta-stimulated protein TSC-22 [Homo sapiens]
MKSQWCRPVAMDLGVYQLRHFSISFLSSLLGTENASVRLDNSSSGASVVAIDNKIEQAMD
LVKSHLMYAVREEVEVLKEQIKELIEKNSQLEQENNLLKTLASPEQLAQFQAQLQTGSPP
ATTQPQGTTQPPAQPASQGSGPTA
>gi|24432096|gb|NM_152912.2|MTIF3 1693 bp mRNA Homo sapiens
mitochondrial translational initiation factor 3 (MTIF3),
mRNA.
GCAGATCCGCTGTACTTGCGGGCGCTACAGTATGTCAATCGCTTGCCCCAGCACAGTGGG
CTCCGTGGCTTAAGACTTGAACCAAGTAAACGAAGTTCTCTTACTGAGAAGTCTCAGTTT
CAAAAGAGCTTCTCCTCATCAACTGGGGATGATTACAGTTCTTCCTAAAAAAGCCTACTT
GATGTGAAGACAATGAGGATGAAGACCTTTATGGTGATCCACTTCCACTTAATAGGATGG
CTGCTCTTTTTCTAAAGAGGTTAACACTACAAACTGTAAAGTCTGAAAATAGTTGCATTA
GATGTTTTGGTAAACACATCCTGCAAAAGACAGCACCAGCACAGTTGTCCCCTATTGCTT
CTGCCCCAAGACTCTCCTTCCTAATTCATGCAAAAGCCTTTAGTACCGCTGAAGACACCC
AGAATGAAGGAAAAAAGACAAAAAAGAATAAAACAGCTTTTAGTAACGTTGGAAGAAAAA
TTAGTCAGCGAGTTATTCACTTATTTGATGAGAAGGGCAATGATTTGGGAAACATGCACC
GAGCAAATGTGATTAGACTTATGGATGAGCGAGACCTGCGACTGGTTCAAAGGAACACCA
GCACAGAACCTGCAGAGTATCAGCTCATGACAGGATTGCAGATCCTCCAGGAGCGGCAGA
GGCTGAGGGAGATGGAGAAGGCGAACCCCAAAACTGGACCAACCCTGAGAAAGGAACTGA
TTTTGTCTTCAAATATTGGACAACATGATTTGGACACAAAGACTAAACAGATTCAGCAGT
GGATTAAGAAAAAACACCTAGTCCAGATTACCATAAAGAAAGGAAAAAATGTAGACGTGT
CAGAAAATGAAATGGAGGAGATATTTCATCAAATACTCCAGACTATGCCTGGAATAGCTA
CATTCTCATCTAGGCCACAAGCTGTTCAAGGAGGAAAAGCTTTAATGTGTGTTCTTCGTG
CTTTGAGCAAAAATGAGGAGAAGGCATATAAAGAAACTCAAGAGACCCAGGAAAGAGACA
CTTTGAACAAAGATCATGGAAATGATAAGGAATCAAATGTTCTGCATCAGTAATTTTAAT
AAAGAAAAGCATGCTCTGAGAGAAAAAAAAGCTCGCTCCTTGGTCTGCAGTCCTTTAAAC
AAAGCAGTGCAGTTCTTAGCCAAGGGTAAGTACTGCAACTGTCGAGAGCATCTTGTCTTC
CACACAGTTGGGTGACTCTCCGTTTTGACACAAAGATAAGCCTTGCCCTTGTTTCCTTTT
GGGAGGGATATATCCACTGAGATGAGAGGCCAAACTCCGTTTTTCACGAGATTTTTTGAC
TTTGAGCTTCATTTTCTTCTTGTCAGGATCATGTACAACAGCATGCCTAGTGAGACTTTG
TTTCATTGCAAATGTTTTGCCACAGCCAGCATGTTCACACACAAAAGGGCGGCTTTCCTC
ATGGAAGGAGAGGATATGGCTTTGGAGATTAAACACAGTTGTATAGGTTCTTCCACAGCC
TTCTCTTGGACAGCGACATAATCCCTTCTGGGGCATGAGTTTATGTGTTGCTTAAGGAAC
TTGCGTTAAAGTTTTCCGGCAACTTCACATGGATTCCTTTGAATGAGTTCAAATGTTCCC
ATGCTAAGCTGAGTCTGTGCCATAGCAAACCATGATATAGCAAGTCTCCAGAATGTGTAC
GAATCAATACTCC
>gi|23097266|gb|NP_690876.1|MTIF3 278 aa linear mitochon-
drial translational initiation factor 3 [Homo sapiens].
MAALFLKRLTLQTVKSENSCIRCFGKHILQKTAPAQLSPIASAPRLSFLIHAKAFSTAED
TQNEGKKTKKNKTAFSNVGRKISQRVIHLFDEKGNDLGNMHRA1NIRLMDERDLRLVQRN
TSTEPAEYQLMTGLQILQERQRLREMEKANPKTGPTLRKELILSSNIGQHDLDTKTKQIQ
QWIKKKHLVQITIKKGKNVDVSENEMEEIFHQILQTMPGIATFSSRPQAVQGGKALMCVL
RALSKNEEKAYKETQETQERDTLNKDHGNDKESNVLHQ
>gi|27499034|gb|XM_044349.7|CAMK2G 1776 bp mRNA Homo sapiens
calcium/calmodulin-dependent protein kinase (CaM kinase) II
gamma (CAMK2G), mRNA.
CAGCATGGCCACCACCGCCACCTGCACCCGTTTCACCGACGACTACCAGCTCTTCGAGGA
GCTTGGCAAGGGTGCTTTCTCTGTGGTCCGCAGGTGTGTGAAGAAAACCTCCACGCAGGA
GTACGCAGCAAAAATCATCAATACCAAGAAGTTGTCTGCCCGGGATCACCAGAAACTAGA
ACGTGAGGCTCGGATATGTCGACTTCTGAAACATCCAAACATCGTGCGCCTCCATGACAG
TATTTCTGAAGAAGGGTTTCACTACCTCGTGTTTGACCTTGTTACCGGCGGGGAGCTGTT
TGAAGACATTGTGGCCAGAGAGTACTACAGTGAAGCAGATGCCAGCCACTGTATACATCA
GATTCTGGAGAGTGTTAACCACATCCACCAGCATGACATCGTCCACAGGGACCTGAAGCC
TGAGAACCTGCTGCTGGCGAGTAAATGCAAGGGTGCCGCCGTCAAGCTGGCTGATTTTGG
CCTAGCCATCGAAGTACAGGGAGAGCAGCAGGCTTGGTTTGGTTTTGCTGGCACCCCAGG
TTACTTGTCCCCTGAGGTCTTGAGGAAAGATCCCTATGGAAAACCTGTGGATATCTGGGC
CTGCGGGGTCATCCTGTATATCCTCCTGGTGGGCTATCCTCCCTTCTGGGATGAGGATCA
GCACAAGCTGTATCAGCAGATCAAGGCTGGAGCCTATGATTTCCCATCACCAGAATGGGA
CACGGTAACTCCTGAAGCCAAGAACTTGATCAACCAGATGCTGACCATAAACCCAGCAAA
GCGCATCACGGCTGACCAGGCTCTCAAGCACCCGTGGGTCTGTCAACGATCCACGGTGGC
ATCCATGATGCATCGTCAGGAGACTGTGGAGTGTTTGCGCAAGTTCAATGCCCGGAGAAA
ACTGAAGGGTGCCATCCTCACGACCATGCTTGTCTCCAGGAACTTCTCAGCTGCCAAAAG
CCTATTGAACAAGAAGTCGGATGGCGGTGTCAAGCCACAGAGCAACAACAAAAACAGTCT
CGTAAGCCCAGCCCAAGAGCCCGCGCCCTTGCAGACGGCCATGGAGCCACAAACCACTGT
GGTACACAACGCTACAGATGGGATCAAGGGCTCCACAGAGAGCTGCAACACCACCACAGA
AGATGAGGACCTCAAAGTGCGAAAACAGGAGATCATTAAGATTACAGAACAGCTGATTGA
AGCCATCAACAATGGGGACTTTGAGGCCTACACGAAGATTTGTGATCCAGGCCTCACTTC
CTTTGAGCCTGAGGCCCTTGGTAACCTCGTGGAGGGGATGGATTTCCATAAGTTTTACTT
TGAGAATCTCCTGTCCAAGAACAGCAAGCCTATCCATACCACCATCCTAAACCCACACGT
CCACGTGATTGGGGAGGACGCAGCGTGCATCGCCTACATCCGCCTCACCCAGTACATCGA
CGGGCAGGGTCGGCCTCGCACCAGCCAGTCAGAAGAGACCCGGGTCTGGCACCGTCGGGA
TGGCAAGTGGCTCAATGTCCACTATCACTGCTCAGGGGCCCCTGCCGCACCGCTGCAGTG
AGCTCAGCCACAGGGGCTTTAGGAGATTCCAGCCGGAGGTCCAACCTTCGCAGCCAGTGG
CTCTGGAGGGCCTGAGTGACAGCGGCAGTCCTGTTTGTTTGAGGTTTAAAACAATTCAAT
TACAAAAGCGGCAGCAGCCAATGCACGCCCCTGCATGCAGCCCTCCCGCCCGCCCTTCGT
GTCTGTCTCTGCTGTACCGAGGTGTTTTTTACATTT
>gi|27499035|gb|XP_044349.7|CAMK2G 518 aa linear similar to
calcium/calmodulin-dependent protein kinase II gamma [Mus
musculus][Homo sapiens].
MATTATCTRFTDDYQLFEELGKGAFSVVRRCVKKTSTQEYAAKIINTKKLSARDHQKLER
EARICRLLKHPNIVRLHDSISEEGFHYLVFDLVTGGELFEDIVAREYYSEADASHCIHQI
LESVNHIHQHDIVHRDLKPENLLLASKCKGAAVKLADFGLAIEVQGEQQAWFGFAGTPGY
LSPEVLRKDPYGKPVDIWACGVILYILLVGYPPFWDEDQHKLYQQIKAGAYDFPSPEWDT
VTPEAKNLINQMLTINPAKRITADQALKHPWVCQRSTVASMMHRQETVECLRKFNARRKL
KGAILTTMLVSRNFSAAKSLLNKKSDGGVKPQSNNKNSLVSPAQEPAPLQTAMEPQTTVV
HNATDGIKGSTESCNTTTEDEDLKVRKQEIIKITEQLIEAINNGDFEAYTKICDPGLTSF
EPEALGNLVEGMDFHKFYFENLLSKNSKPIHTTILNPHVHVIGEDAACIAYIRLTQYIDG
QGRPRTSQSEETRVWHRRDGKWLNVHYHCSGAPAAPLQ
>gi|5453881|gb|NM_006213.1|PHKG1 1377 bp mRNA Homo sapiens
phosphorylase kinase, gamma 1 (muscle) (PHKG1), mRNA.
GGCCTTCAGCCCTCTGTGGTCCCCTCTCCCCGGGGGGCTTTGGGATTCTTGTCAAGCTCC
TTCAAGAGCCTGCAAGCACTTAACCAGCCACCCAGAGTTCCCTCACTGAAGATCTGAGCA
TGACCCGGGACGAGGCACTGCCGGACTCTCATTCTGCACAGGACTTCTATGAGAATTATG
AGCCCAAAGAGATCCTGGGCAGGGGCGTTAGCAGTGTGGTCAGGCGATGCATCCACAAGC
CCACGAGCCAGGAGTACGCCGTGAAGGTCATCGACGTCACCGGTGGAGGCAGCTTGAGCC
CGGAGGAGGTGCGGGAGCTGCGAGAAGCCACGCTGAAGGAGGTGGACATCCTGCGCAAGG
TCTCAGGGCACCCCAACATCATACAGCTGAAGGACACTTATGAGACCAACACTTTCTTCT
TCTTGGTGTTTGACCTGATGAAGAGAGGGGAGCTCTTTGACTACCTCACTGAGAAGGTCA
CCTTGAGTGAGAAGGAAACCAGAAAGATCATGCGAGCTCTGCTGGAGGTGATCTGCACCT
TGCACAAACTCAACATCGTGCACCGGGACCTGAAGCCCGAGAACATTCTCTTGGATGACA
ACATGAACATCAAGCTCACAGACTTTGGCTTTTCCTGCCAGCTGGAGCCGGGAGAGAGGC
TGCGAGAGGTCTGCGGGACCCCCAGTTACCTGGCCCCTGAGATTATCGAGTGCTCCATGA
ATGAGGACCACCCGGGCTACGGGAAAGAGGTGGACATGTGGAGCACTGGCGTCATCATGT
ACACGCTGCTGGCCGGCTCCCCGCCCTTCTGGCACCGGAAGCAGATGCTGATGCTGAGGA
TGATCATGAGCGGCAACTACCAGTTTGGCTCGCCCGAGTGGGATGATTACTCGGACACCG
TGAAGGACCTGGTCTCCCGATTCCTGGTGGTGCAACCCCAGAACCGCTACACAGCGGAAG
AGGCCTTGGCACACCCCTTCTTCCAGCAGTACTTGGTGGAGGAAGTGCGGCACTTCAGCC
CCCGGGGGAAGTTCAAGGTGATCGCTCTGACCGTGCTGGCTTCAGTGCGGATCTACTACC
AGTACCGCCGGGTGAAGCCTGTGACCCGGGAGATCGTCATCCGAGACCCCTATGCCCTCC
GGCCTCTGCGCCGGCTCATCGACGCCTACGCTTTCCGAATCTATGGCCACTGGGTGAAGA
AGGGGCAGCAGCAGAACCGGGCAGCCCTTTTCGAGAACACACCCAAGGCCGTGCTCCTCT
CCCTGGCCGAGGAGGACTACTGAGGGGCTGGCCAGTCAGGGAGGGCTAGGGGGCAGGTGG
GGAGGGGAAGCCATGGAAATACAAGTCAAAGGGGTAAAAAAAAAAAAAAAAAAAAAA
>gi|5453882|gb|NP_006204.1|PHKG1|387 aa linear phosphory-
lase kinase, gamma 1 (muscle) [Homo sapiens]
MTRDEALPDSHSAQDFYENYEPKEILGRGVSSVVRRCIHKPTSQEYAVKVIDVTGGGSFS
PEEVRELREATLKEVDILRKVSGHPNIIQLKDTYETNTFFFLVFDLMKPGELFDYLTEKV
TLSEKETRKIMRALLEVICTLHKLNIVHRDLKPENILLDDNMNIKLTDFGFSCQLEPGER
LREVCGTPSYLAPEIIECSMNEDHPGYGKEVDMWSTGVIMYTLLAGSPPFWHRKQMLMLR
MIMSGNYQFGSPEWDDYSDTVKDLVSRFLVVQPQNRYTAEEALAHPFFQQYLVEEVRHFS
PRGKFKVIALTVLASVRIYYQYRRVKPVTREIVIRDPYALRPLRRLIDAYAFRIYGHWVK
KGQQQNRAALFENTPKAVLLSLAEEDY
>gi|4503412|gb|NM_001945.1|DTR 2360 bp mRNA Homo sapiens
diphtheria toxin receptor (heparin-binding epidermal growth
factor-like growth factor). (DTR), mRNA.
GCTACGCGGGCCACGCTGCTGGCTGGCCTGACCTAGGCGCGCGGGGTCGGGCGGCCGCGC
GGGCGGGCTGAGTGAGCAAGACAAGACACTCAAGAAGAGCGAGCTGCGCCTGGGTCCCGG
CCAGGCTTGCACGCAGAGGCGGGCGGCAGACGGTGCCCGGCGGAATCTCCTGAGCTCCGC
CGCCCAGCTCTGGTGCCAGCGCCCAGTGGCCGCCGCTTCGAAAGTGACTGGTGCCTCGCC
GCCTCCTCTCGGTGCGGGACCATGAAGCTGCTGCCGTCGGTGGTGCTGAAGCTCTTTCTG
GCTGCAGTTCTCTCGGCACTGGTGACTGGCGAGAGCCTGGAGCGGCTTCGGAGAGGGCTA
GCTGCTGGAACCAGCAACCCGGACCCTCCCACTGTATCCACGGACCAGCTGCTACCCCTA
GGAGGCGGCCGGGACCGGAAAGTCCGTGACTTGCAAGAGGCAGATCTGGACCTTTTGAGA
GTCACTTTATCCTCCAAGCCACAAGCACTGGCCACACCAAACAAGGAGGAGCACGGGAAA
AGAAAGAAGAAAGGCAAGGGGCTAGGGAAGAAGAGGGACCCATGTCTTCGGAAATACAAG
GACTTCTGCATCCATGGAGAATGCAAATATGTGAAGGAGCTCCGGGCTCCCTCCTGCATC
TGCCACCCGGGTTACCATGGAGAGAGGTGTCATGGGCTGAGCCTCCCAGTGGAAAATCGC
TTATATACCTATGACCACACAACCATCCTGGCCGTGGTGGCTGTGGTGCTGTCATCTGTC
TGTCTGCTGGTCATCGTGGGGCTTCTCATGTTTAGGTACCATAGGAGAGGAGGTTATGAT
GTGGAAAATGAAGAGAAAGTGAAGTTGGGCATGACTAATTCCCACTGAGAGAGACTTGTG
CTCAAGGAATCGGCTGGGGACTGCTACCTCTGAGAAGACACAAGGTGATTTCAGACTGCA
GAGGGGAAAGACTTCCATCTAGTCACAAAGACTCCTTCGTCCCCAGTTGCCGTCTAGGAT
TGGGCCTCCCATAATTGCTTTGCCAAAATACCAGAGCCTTCAAGTGCCAAACAGAGTATG
TCCGATGGTATCTGGGTAAGAAGAAAGCAAAAGCAAGGGACCTTCATGCCCTTCTGATTC
CCCTCCACCAAACCCCACTTCCCCTCATAAGTTTGTTTAAACACTTATCTTCTGGATTAG
AATGCCGGTTAAATTCCATATGCTCCAGGATCTTTGACTGAAAAAAAAAAAGAAGAAGAA
GAAGGAGAGCAAGAAGGAAAGATTTGTGAACTGGAAGAAAGCAACAAAGATTGAGAAGCC
ATGTACTCAAGTACCACCAAGGGATCTGCCATTGGGACCCTCCAGTGCTGGATTTGATGA
GTTAACTGTGAAATACCACAAGCCTGAGAACTGAATTTTGGGACTTCTACCCAGATGGAA
AAATAACAACTATTTTTGTTGTTGTTGTTTGTAAATGCCTCTTAAATTATATATTTATTT
TATTCTATGTATGTTAATTTATTTAGTTTTTAACAATCTAACAATAATATTTCAAGTGCC
TAGACTGTTACTTTGGCAATTTCCTGGCCCTCCACTCCTCATCCCCACAATCTGGCTTAG
TGCCACCCACCTTTGCCACAAAGCTAGGATGGTTCTGTGACCCATCTGTAGTAATTTATT
GTCTGTCTACATTTCTGCAGATCTTCCGTGGTCAGAGTGCCACTGCGGGAGCTCTGTATG
GTCAGGATGTAGGGGTTAACTTGGTCAGAGCCACTCTATGAGTTGGACTTCAGTCTTGCC
TAGGCGATTTTGTCTACCATTTGTGTTTTGAAAGCCCAAGGTGCTGATGTCAAAGTGTAA
CAGATATCAGTGTCTCCCCGTGTCCTCTCCCTGCCAAGTCTCAGAAGAGGTTGGGCTTCC
ATGCCTGTAGCTTTCCTGGTCCCTCACCCCCATGGCCCCAGGCCACAGCGTGGGAACTCA
CTTTCCCTTGTGTCAAGACATTTCTCTAACTCCTGCCATTCTTCTGGTGCTACTCCATGC
AGGGGTCAGTGCAGCAGAGGACAGTCTGGAGAAGGTATTAGCAAAGCAAAAGGCTGAGAA
GGAACAGGGAACATTGGAGCTGACTGTTCTTGGTAACTGATTACCTGCCAATTGCTACCG
AGAAGGTTGGAGGTGGGGAAGGCTTTGTATAATCCCACCCACCTCACCAAAACGATGAAG
GTATGCTGTCATGGTCCTTTCTGGAAGTTTCTGGTGCCATTTCTGAACTGTTACAACTTG
TATTTCCAAACCTGGTTCATATTTATACTTTGCAATCCAAATAAAGATAACCCTTATTCC
ATAAAAAAAAAAAAAAAAAA
>gi|4503413|gb|NP_001936.1|DTR 208 aa linear diphtheria
toxin receptor (heparin-binding epidermal growth factor-like
growth factor); Diphtheria toxin receptor (heparin-binding
EGF-like growth factor) [Homo sapiens].
MKLLPSVVLKLFLAAVLSALVTGESLERLRRGLAAGTSNPDPPTVSTDQLLPLGGGRDRK
VRDLQEADLDLLRVTLSSKPQALATPNKEEHGKRKKKGKGLGKKRDPCLRKYKDFCIHGE
CKYVKELRAPSCICHPGYHGERCHGLSLPVENRLYTYDHTTILAVVAVVLSSVCLLVIVG
LLMFRYHRRGGYDVENEEKVKLGMTNSH
>gi|4507460|gb|NM_003236.1|TGFA 4119 bp mRNA Homo sapiens
transforming growth factor, alpha (TGFA), mRNA.
CTGGAGAGCCTGCTGCCCGCCCGCCCGTAAAATGGTCCCCTCGGCTGGACAGCTCGCCCT
GTTCGCTCTGGGTATTGTGTTGGCTGCGTGCCAGGCCTTGGAGAACAGCACGTCCCCGCT
GAGTGCAGACCCGCCCGTGGCTGCAGCAGTGGTGTCCCATTTTAATGACTGCCCAGATTC
CCACACTCAGTTCTGCTTCCATGGAACCTGCAGGTTTTTGGTGCAGGAGGACAAGCCAGC
ATGTGTCTGCCATTCTGGGTACGTTGGTGCACGCTGTGAGCATGCGGACCTCCTGGCCGT
GGTGGCTGCCAGCCAGAAGAAGCAGGCCATCACCGCCTTGGTGGTGGTCTCCATCGTGGC
CCTGGCTGTCCTTATCATCACATGTGTGCTGATACACTGCTGCCAGGTCCGAAAACACTG
TGAGTGGTGCCGGGCCCTCATCTGCCGGCACGAGAAGCCCAGCGCCCTCCTGAAGGGAAG
AACCGCTTGCTGCCACTCAGAAACAGTGGTCTGAAGAGCCCAGAGGAGGAGTTTGGCCAG
GTGGACTGTGGCAGATCAATAAAGAAAGGCTTCTTCAGGACAGCACTGCCAGAGATGCCT
GGGTGTGCCACAGACCTTCCTACTTGGCCTGTAATCACCTGTGCAGCCTTTTGTGGGCCT
TCAAAACTCTGTCAAGAACTCCGTCTGCTTGGGGTTATTCAGTGTGACCTAGAGAAGAAA
TCAGCGGACCACGATTTCAAGACTTGTTAAAAAAGAACTGCAAAGAGACGGACTCCTGTT
CACCTAGGTGAGGTGTGTGCAGCAGTTGGTGTCTGAGTCCACATGTGTGCAGTTGTCTTC
TGCCAGCCATGGATTCCAGGCTATATATTTCTTTTTAATGGGCCACCTCCCCACAACAGA
ATTCTGCCCAACACAGGAGATTTCTATAGTTATTGTTTTCTGTCATTTGCCTACTGGGGA
AGAAAGTGAAGGAGGGGAAACTGTTTAATATCACATGAAGACCCTAGCTTTAAGAGAAGC
TGTATCCTCTAACCACGAGACTCTCAACCAGCCCAACATCTTCCATGGACACATGACATT
GAAGACCATCCCAAGCTATCGCCACCCTTGGAGATGATGTCTTATTTATTAGATGGATAA
TGGTTTTATTTTTAATCTCTTAAGTCAATGTAAAAAGTATAAAACCCCTTCAGACTTCTA
CATTAATGATGTATGTGTTGCTGACTGAAAAGCTATACTGATTAGAAATGTCTGGCCTCT
TCAAGACAGCTAAGGCTTGGGAAAAGTCTTCCAGGGTGCGGAGATGGAACCAGAGGCTGG
GTTACTGGTAGGAATAAAGGTAGGGGTTCAGAAATGGTGCCATTGAAGCCACAAAGCCGG
TAAATGCCTCAATACGTTCTGGGAGAAAACTTAGCAAATCCATCAGCAGGGATCTGTCCC
CTCTGTTGGGGAGAGAGGAAGAGTGTGTGTGTCTACACAGGATAAACCCAATACATATTG
TACTGCTCAGTGATTAAATGGGTTCACTTCCTCGTGAGCCCTCGGTAAGTATGTTTAGAA
ATAGAACATTAGCCACGAGCCATAGGCATTTCAGGCCAAATCCATGAAAGGGGGACCAGT
CATTTATTTTCCATTTTGTTGCTTGGTTGGTTTGTTGCTTTATTTTTAAAAGGAGAAGTT
TAACTTTGCTATTTATTTTCGAGCACTAGGAAAACTATTCCAGTAATTTTTTTTTCCTCA
TTTCCATTCAGGATGCCGGCTTTATTAACAAAAACTCTAACAAGTCACCTCCACTATGTG
GGTCTTCCTTTCCCCTCAAGAGAAGGAGCAATTGTTCCCCTGACATCTGGGTCCATCTGA
CCCATGGGGCCTGCCTGTGAGAAACAGTGGGTCCCTTCAAATACATAGTGGATAGCTCAT
CCCTAGGAATTTTCATTAAAATTTGGAAACAGAGTAATGAAGAAATAATATATAAACTCC
TTATGTGAGGAAATGCTACTAATATCTGAAAAGTGAAAGATTTCTATGTATTAACTCTTA
AGTGCACCTAGCTTATTACATCGTGAAAGGTACATTTAAAATATGTTAAATTGGCTTGAA
ATTTTCAGAGAATTTTGTCTTCCCCTAATTCTTCTTCCTTGGTCTGGAAGAACAATTTCT
ATGAATTTTCTCTTTATTTTTTTTTTATAATTCAGACAATTCTATGACCCGTGTCTTCAT
TTTTGGCACTCTTATTTAACAATGCCACACCTGAAGCACTTGGATCTGTTCAGAGCTGAC
CCCCTAGCAACGTAGTTGACACAGCTCCAGGTTTTTAAATTACTAAAATAAGTTCAAGTT
TACATCCCTTGGGCCAGATATGTGGGTTGAGGCTTGACTGTAGCATCCTGCTTAGAGACC
AATCAATGGACACTGGTTTTTAGACCTCTATCAATCAGTAGTTAGCATCCAAGAGACTTT
GCAGAGGCGTAGGATGAAGGCTGGACAGATGGCGGAACGAGAGGTTCCCTGCGAAGACTT
GAGATTTAGTGTCTGTGAATGTTCTAGTTCCTAGGTCCAGCAAGTCACACCTGCCAGTGC
CCTCATCCTTATGCCTGTAACACACATGCAGTGAGAGGCCTCACATATACGCCTCCCTAG
AAGTGCCTTCCAAGTCAGTCCTTTGGAAACCAGCAGGTCTGAAAAAGAGGCTGCATCAAT
GCAAGCCTGGTTGGACCATTGTCCATGCCTCAGGATAGAACAGCCTGGCTTATTTGGGGA
TTTTTCTTCTAGAAATCAAATGACTGATAAGCATTGGCTCCCTCTGCCATTTAATGGCAA
TGGTAGTCTTTGGTTAGCTGCAAAAATACTCCATTTCAAGTTAAAAATGCATCTTCTAAT
CCATCTCTGCAAGCTCCCTGTGTTTCCTTGCCCTTTAGAAAATGAATTGTTCACTACAAT
TAGAGAATCATTTAACATCCTGACCTGGTAAGCTGCCACACACCTGGCAGTGGGGAGCAT
CGCTGTTTCCAATGGCTCAGGAGACAATGAAAAGCCCCCATTTAAAAAAATAACAAACAT
TTTTTAAAAGGCCTCCAATACTCTTATGGAGCCTGGATTTTTCCCACTGCTCTACAGGCT
GTGACTTTTTTTAAGCATCCTGACAGGAAATGTTTTCTTCTACATGGAAAGATAGACAGC
AGCCAACCCTGATCTGGAAGACAGGGCCCCGGCTGGACACACGTGGAACCAAGCCAGGGA
TGGGCTGGCCATTGTGTCCCCGCAGGAGAGATGGGCAGAATGGCCCTAGAGTTCTTTTCC
CTGAGAAAGGAGAAAAAGATGGGATTGCCACTCACCCACCCACACTGGTAAGGGAGGAGA
ATTTGTGCTTCTGGAGCTTCTCAAGGGATTGTGTTTTGCAGGTACAGAAAACTGCCTGTT
ATCTTCAAGCCAGGTTTTCGAGGGCACATGGGTCACCAGTTGCTTTTTCAGTCAATTTGG
CCGGGATGGACTAATGAGGCTCTAACACTGCTCAGGAGACCCCTGCCCTCTAGTTGGTTC
TGGGCTTTGATCTCTTCCAACCTGCCCAGTCACAGAAGGAGGAATGACTCAAATGCCCAA
AACCAAGAACACATTGCAGAAGTAAGACAAACATGTATATTTTTAAATGTTCTAACATAA
GACCTGTTCTCTCTAGCCATTGATTTACCAGGCTTTCTGAAAGATCTAGTGGTTCACACA
GAGAGAGAGAGAGTACTGAAAAAGCAACTCCTCTTCTTAGTCTTAATAATTTACTAAAAT
GGTCAACTTTTCATTATCTTTATTATAATAAACCTGATGCTTTTTTTTAGAACTCCTTAC
TCTGATGTCTGTATATGTTGCACTGAAAAGGTTAATATTTAATGTTTTAATTTATTTTGT
GTGGTAAGTTAATTTTGATTTCTGTAATGTGTTAATGTGATTAGCAGTTATTTTCCTTAA
TATCTGAATTATACTTAAAGAGTAGTGAGCAATATAAGACGCAATTGTGTTTTTCAGTAA
TGTGCATTGTTATTGAGTTGTACTGTACCTTATTTGGAAGGATGAAGGAATGAACCTTTT
TTTCCTAAAAAAAAAAAAAAAAAAAAAAAAAAAAAAAAA
>gi|4507461|gb|NP_003227.1|TGFA 160 aa linear transforming
growth factor, alpha [Homo sapiens].
MVPSAGQLALFALGIVLAACQALENSTSPLSADPPVAAAVVSHFNDCPDSHTQFCFHGTC
RFLVQEDKPACVCHSGYVGARCEHADLLAVVAAAQKKQAITALVVVSIVALAVLIITCVL
IHCCQVRKHCEWCRALICRHEKPSALLKGRTACCHSETVV
>gi|6912653|gb|NM_012433.1|SF3B1 4259 bp mRNA Homo sapiens
splicing factor 3b, subunit 1, 155kDa (SF3B1), mRNA.
ATGGCGAAGATCGCCAAGACTCACGAAGATATTGAAGCACAGATTCGAGAAATTCAAGGC
AAGAAGGCAGCTCTTGATGAAGCTCAAGGAGTGGGCCTCGATTCTACAGGTTATTATGAC
CAGGATTTATGGTGGAGTGACAGCAGATTTGCTGGATACGTGACATCAATTGCTGCTGCA
ACTGAACTTGAAGATGATGACGATGACTATTCATCATCTACGAGTTTGCTTGGTCAGAAG
AAGCCAGGATATCATGCCCCTGTGGCATTGCTTAATGATATACCACAGTCAACAGAACAG
TATGATCCATTTGCTGAGCACAGACCTCCAAAGATTGCAGACCGGGAAGATGAATACAAA
AAGCATAGGCGGACCATGATAATTTCCCCAGAGCGTCTTGATCCTTTTGCAGATGGAGGG
AAGACCCCTGATCCTAAAATGAATGTTAGGACTTACATGGATGTAATGCGAGAACAACAC
TTGACTAAAGAAGAACGAGAAATTAGGCAACAGCTAGCAGAAAAAGCTAAAGCTGGAGAA
CTAAAAGTCGTCAATGGAGCAGCAGCGTCCCAGCCTCCATCAAAACGAAAACGGCGTTGG
GATCAAACAGCTGATCAGACTCCTGGTGCCACTCCCAAAAAACTATCAAGTTGGGATCAG
GCAGAGACCCCTGGGCATACTCCTTCCTTAAGATGGGATGAGACACCAGGTCGTGCAAAG
GGAAGCGAGACTCCTGGAGCAACCCCAGGCTCAAAAATATGGGATCCTACACCTAGCCAC
ACACCAGCGGGAGCTGCTACTCCTGGACGAGGTGATACACCAGGCCATGCGACACCAGGC
CATGGAGGCGCAACTTCCAGTGCTCGTAAAAACAGATGGGATGAAACCCCCAAAACAGAG
AGAGATACTCCTGGGCATGGAAGTGGATGGGCTGAGACTCCTCGAACAGATCGAGGTGGA
GATTCTATTGGTGAAACACCGACTCCTGGAGCCAGTAAAAGAAAATCACGGTGGGATGAA
ACACCAGCTAGTCAGATGGGTGGAAGCACTCCAGTTCTGACCCCTGGAAAGACACCAATT
GGCACACCAGCCATGAACATGGCTACCCCTACTCCAGGTCACATAATGAGTATGACTCCT
GAACAGCTTCAGGCTTGGCGGTGGGAAAGAGAAATTGATGAGAGAAATCGCCCACTTTCT
GATGAGGAATTAGATGCTATGTTCCCAGAAGGATATAAGGTACTTCCTCCTCCAGCTGGT
TATGTTCCTATTCGAACTCCAGCTCGAAAGCTGACAGCTACTCCAACACCTTTGGGTGGT
ATGACTGGTTTCCACATGCAAACTGAAGATCGAACTATGAAAAGTGTTAATGACCAGCCA
TCTGGAAATCTTCCATTTTTAAAACCTGATGATATTCAATACTTTGATAAACTATTGGTT
GATGTTGATGAATCAACACTTAGTCCAGAAGAGCAAAAAGAGAGAAAAATAATGAAGTTG
CTTTTAAAAATTAAGAATGGAACACCACCAATGAGAAAGGCTGCATTGCGTCAGATTACT
GATAAAGCTCGTGAATTTGGAGCTGGTCCTTTGTTTAATCAGATTCTTCCTCTGCTGATG
TCTCCTACACTTGAGGATCAAGAGCGTCATTTACTTGTGAAAGTTATTGATAGGATACTG
TACAAACTTGATGACTTAGTTCGTCCATATGTGCATAAGATCCTCGTGGTCATTGAACCG
CTATTGATTGATGAAGATTACTATGCTAGAGTGGAAGGCCTAGAGATCATTTCTAATTTG
GCAAAGGCTGCTGGTCTGGCTACTATGATCTCTACCATGAGACCTGATATAGATAACATG
GATGAGTATGTCCGTAACACAACAGCTAGAGCTTTTGCTGTTGTAGCCTCTGCCCTGGGC
ATTCCTTCTTTATTGCCCTTCTTAAAAGCTGTGTGCAAAAGCAAGAAGTCCTGGCAAGCG
AGACACACTGGTATTAAGATTGTACAACAGATAGCTATTCTTATGGGCTGTGCCATCTTG
CCACATCTTAGAAGTTTAGTTGAAATCATTGAACATGGTCTTGTGGATGAGCAGCAGAAA
GTTCGGACCATCAGTGCTTTGGCCATTGCTGCCTTGGCTGAAGCAGCAACTCCTTATGGT
ATCGAATCTTTTGATTCTGTGTTAAAGCCTTTATGGAAGGGTATCCGCCAACACAGAGGA
AAGGGTTTGGCTGCTTTCTTGAAGGCTATTGGGTATCTTATTCCTCTTATGGATGCAGAA
TATGCCAACTACTATACTAGAGAAGTGATGTTAATCCTTATTCGAGAATTCCAGTCTCCT
GATGAGGAAATGAAAAAAATTGTGCTGAAGGTGGTAAAACAGTGTTGTGGGACAGATGGT
GTAGAAGCAAACTACATTAAAACAGAGATTCTTCCTCCCTTTTTTAAACACTTCTGGCAG
CACAGGATGGCTTTGGATAGAAGAAATTACCGACAGTTAGTTGATACTACTGTGGAGTTG
GCAAACAAAGTAGGTGCAGCAGAAATTATATCCAGGATTGTGGATGATCTGAAAGATGAA
GCCGAACAGTACAGAAAAATGGTGATGGAGACAATTGAGAAAATTATGGGCAATTTGGGA
GCAGCAGATATTGATCATAAACTTGAAGAACAACTGATTGATGGTATTCTTTATGCTTTC
CAAGAACAGACTACAGAGGACTCAGTAATGTTGAACGGCTTTGGCACAGTGGTTAATGCT
CTTGGCAAACGAGTCAAACCATACTTGCCTCAGATCTGTGGTACAGTTTTGTGGCGTTTA
AATAACAAATCTGCTAAAGTTAGGCAACAGGCAGCTGACTTGATTTCTCGAACTGCTGTT
GTCATGAAGACTTGTCAAGAGGAAAAATTGATGGGACACTTGGGTGTTGTATTGTATGAG
TATTTGGGTGAAGAGTACCCTGAAGTATTGGGCAGCATTCTTGGAGCACTGAAGGCCATT
GTAAATGTCATAGGTATGCATAAGATGACTCCACCAATTAAAGATCTGCTGCCTAGACTC
ACCCCCATCTTAAAGAACAGACATGAAAAAGTACAAGAGAATTGTATTGATCTTGTTGGT
CGTATTGCTGACAGGGGAGCTGAATATGTATCTGCAAGAGAGTGGATGAGGATTTGCTTT
GAGCTTTTAGAGCTCTTAAAAGCCCACAAAAAGGCTATTCGTAGAGCCACAGTCAACACA
TTTGGTTATATTGCAAAGGCCATTGGCCCTCATGATGTATTGGCTACACTTCTGAACAAC
CTCAAAGTTCAAGAAAGGCAGAACAGAGTTTGTACCACTGTAGCAATAGCTATTGTTGCA
GAAACATGTTCACCCTTTACAGTACTCCCTGCCTTAATGAATGAATACAGAGTTCCTGAA
CTGAATGTTCAAAATGGAGTGTTAAAATCGCTTTCCTTCTTGTTTGAATATATTGGTGAA
ATGGGAAAAGACTACATTTATGCCGTAACACCGTTACTTGAAGATGCTTTAATGGATAGA
GACCTTGTACACAGACAGACGGCTAGTGCAGTGGTACAGCACATGTCACTTGGGGTTTAT
GGATTTGGTTGTGAAGATTCGCTGAATCACTTGTTGAACTATGTATGGCCCAATGTATTT
GAGACATCTCCTCATGTAATTCAGGCAGTTATGGGAGCCCTAGAGGGCCTGAGAGTTGCT
ATTGGACCATGTAGAATGTTGCAATATTGTTTACAGGGTCTGTTTCACCCAGCCCGGAAA
GTCAGAGATGTATATTGGAAAATTTACAACTCCATCTACATTGGTTCCCAGGACGCTCTC
ATAGCACATTACCCAAGAATCTACAACGATGATAAGAACACCTATATTCGTTATGAACTT
GACTATATCTTATAATTTTATTGTTTATTTTGTGTTTAATGCACAGCTACTTCACACCTT
AAACTTGCTTTGATTTGGTGATGTAAACTTTTAAACATTGCAGTTCAGTGTAGAACTGGT
CATAGAGGAAGAGCTAGAAATCCAGTAGCATGATTTTTAAATAACCTGTCTTTGTTTTTG
ATGTTAAACAGTAAATGCCAGTAGTGACCAAGAACACAGTGATTATATACACTATACTGG
AGGGATTTCATTTTTAATTCATCTTTATGAAGATTTAGAACTCATTCCTTGTGTTTAAAG
GGAATGTTTAATTGAGAAATAAACATTTGTGTACAAAATGCTAAAAAAAAAAAAAAAAA
>gi|6912654|gb|NP_036565.1|SF3B1 1304 aa linear splicing
factor 3b, subunit 1, 155kDa; spliceosome-associated factor
155; splicing factor 3b, subunit 1, 155kD [Homo sapiens].
MAKTAKTHEDIEAQIREIQGKKAALDEAQGVGLDSTGYYDQEIYGGSDSRFAGYVTSIAA
TELEDDDDDYSSSTSLLGQKKPGYHAPVALLNDIPQSTEQYDPFAEHRPPKIADREDEYK
KHRRTMIISPERLDPFADGGKTPDPKMNVRTYMDVMREQHLTKEEREIRQQLAEKAKAGE
LKVVNGAAASQPPSKRKRRWDQTADQTPGATPKKLSSWDQAETPGHTPSLRWDETPGRAK
GSETPGATPGSKIWDPTPSHTPAGAATPGRGDTPGHATPGHGGATSSARKNRWDETPKTE
RDTPGHGSGWAETPRTDRGGDSIGETPTPGASKRKSRWDETPASQMGGSTPVLTPGKTPI
GTPAMNMATPTPGHIMSMTPEQLQAWRWEREIDERNRPLSDEELDAMFPEGYKVLPPPAG
YVPIRTPARKLTATPTPLGGMTGFHMQTEDRTMKSVNDQPSGNLPFLKPDDIQYFDKLLV
DVDESTLSPEEQKERKIMKLLLKIKNGTPPMRKAALRQITDKAREFGAGPLFNQILPLLM
SPTLEDQERRLLVKVIDRILYKLDDLVRPYVHKILVVIEPLLIDEDYYARVEGLEIISNL
AKAAGLATMISTMRPDIDNMDEYVRNTTARAFAVVASALGIPSLLPFLKAVCKSKKSWQA
RHTGIKIVQQIAILMGCAILPHLRSLVEIIEHGLVDEQQKVRTISALAIAALAEAATPYG
IESFDSVLKPLWKGIRQHRGKGLAAFLKAIGYLIPLMDAEYANYYTREVMLILIREFQSP
DEEMKKIVLKVVKQCCGTDGVEANYIKTEILPPFFKHFWQHRMALDRRNYRQLVDTTVEL
ANKVGAAEIISRIVDDLKDEAEQYRKMVMETIEKIMGNLGAADIDHKLEEQLIDGILYAF
QEQTTEDSVMLNGFGTVVNALGKRVKPYLPQICGTVLWRLNNKSAKVRQQAADLISRTAV
VMKTCQEEKLMGHLGVVLYEYLGEEYPEVLGSILGALKAIVNVIGMHKMTPPIKDLLPRL
TPILKNRHEKVQENCIDLVGRIADRGAEYVSAREWMRICFELLELLKAHKKAIRRATVNT
FGYIAKAIGPHDVLATLLNNLKVQERQNRVCTTVAIAIVAETCSPFTVLPALMNEYRVPE
LNVQNGVLKSLSFLFEYIGEMGKDYIYAVTPLLEDALMDRDLVHRQTASAVVQHMSLGVY
GFGCEDSLNRLLNYVWPNVFETSPHVIQAVMGALEGLRVAIGPCRMLQYCLQGLFHPARK
VRDVYWKIYNSIYIGSQDALIAHYPRIYNDDKNTYIRYELDYIL
>gi|21707321|gb|BC033864.1|BC033864 2321 bp mRNA Homo
sapiens, Similar to branched chain aminotransferase 1,
cytosolic, clone MGC:45234 IMAGE:5186262, mRNA, complete
cds.
GGTGGATGCTGCGGCATCGGAGGACCCTGCTGGTGGAGGAAATGGTTCACGCCCGTCCCC
GTTCCCTTTGCAGGCTTGCTATTGTGCGTCTGTGATTGACAAGACCACGAGGCTGAGCGC
GCCCTGGAGATTTTTCTATAAATGGCTTAACACCCCAGTCTAGACTATTTGCTCGGATAT
AAGGGAGACAATTGTTTTTTTGTTCTTTGCCGGCGAACCCTGGCTCTGTAGGGCTGACCT
GGAATTTAACCAGTCTTCCCTGAGCCGGCGGAGGAGGACAAAAACCGCCGCGACCCCGGC
AGGGTGGGAAGTGCAGGGCAGCGCTCCCAAGACACGCTTGTTGGAGGTTCGGGCCTGGGT
GCTTGGTTGTCTGAGCCTCCTTTTTTGTGTTTGCCTGGGTCCTGGAGAGGAGCGCACGGT
ATCATGGATTGCAGTAACGGATGCTCCGCAGAGTGTACCGGAGAAGGAGGATCAAAAGAG
GTGGTGGGGACTTTTAAGGCTAAAGACCTAATAGTCACACCAGCTACCATTTTAAAGGAA
AAACCAGACCCCAATAATCTGGTTTTTGGAACTGTGTTCACGGATCATATGCTGACGGTG
GAGTGGTCCTCAGAGTTTGGATGGGAGAAACCTCATATCAAGCCTCTTCAGAACCTGTCA
TTGCACCCTGGCTCATCAGCTTTGCACTATGCAGTGGAATTATTTGAAGGATTGAAGGCA
TTTCGAGGAGTAGATAATAAAATTCGACTGTTTCAGCCAAACCTCAACATGGATAGAATG
TATCGCTCTGCTGTGAGGGCAACTCTGCCGGTATTTGACAAAGAAGAGCTCTTAGAGTGT
ATTCAACAGCTTGTGAAATTGGATCAAGAATGGGTCCCATATTCAACATCTGCTAGTCTG
TATATTCGTCCTACATTCATTGGAACTGAGCCTTCTCTTGGAGTCAAGAAGCCTACCAAA
GCCCTGCTCTTTGTACTCTTGAGCCCAGTGGGACCTTATTTTTCAAGTGGAACCTTTAAT
CCAGTGTCCCTGTGGGCCAATCCCAAGTATGTAAGAGCCTGGAAAGGTGGAACTGGGGAC
TGCAAGATGGGAGGGAATTACGGCTCATCTCTTTTTGCCCAATGTGAAGCAGTAGATAAT
GGGTGTCAGCAGGTCCTGTGGCTCTATGGAGAGGACCATCAGATCACTGAAGTGGGAACT
ATGAATCTTTTTCTTTACTGGATAAATGAAGATGGAGAAGAAGAACTGGCAACTCCTCCA
CTAGATGGCATCATTCTTCCAGGAGTGACAAGGCGGTGCATTCTGGACCTGGCACATCAG
TGGGACACAGAACTCAGCTTGTTTTCAATTAATTTGCCTGATTTTCTGCAGTTCATTTAC
TTTTGAACAACATAATTGCAATTGTAGACTGAGAGAAATTGAAACTTTCAAAGAGCCATA
TTTCTATTGCAGATATATTTTCCTGCTCTTCCAAATCTACTTACAGCATGAGTTCTTCTT
TTAAATATTCAAATATTTTGAATATTGCCAAGAGCTTTGATTTCCATTTTTATCTCTTGT
GGGTTTATAAATTAAGAAAAAATACTCATCTTATTTTTTTAAACCTCTCTATTTTTATTG
CCCTTTATTCAAATAACTTGTTGACAAACTTTGAACTTGAACCACTGAGGTAAAAGAACA
AGAATTAAACAGATAGTTTAAACACATAGCTTAAAAGGATCTTTTTCCCATTTCCTATCC
TTGAGCAAAGAATATATTCAAACACTTTGGCAGAAGTCAATGAGGTTATACCACTAATTC
CATGATGAAAATCAACTGAATGTGATACTGAAAGAGAAGGAAGAGAATTGTCACTGTAAA
GTCAACTGTTAGTCATATTAGGAAAAAAAATACATACAATACAATTTCTCAAATAAAGTC
CAAATATACATTCAATGTTTAAAAATAATGAGTATTTCAGATATTTGAACTCAGTCTGTT
CTTTATTCCATAAAAGATATAGGTAAGCCGTGCACGGTGGCTCACAACTATAATCCCAGC
ACTTTGGCACTTTGGGAGGCTGAGGTGGGAGGATCACATGAGCCCAGCCTGGGCAACATA
GGGAGACCGCTATCTTTACAAAATAAAATATAAAATATAAAACCTAGTTGGGCATGGCAG
CATACACCTGTAGTCCCAGGTGCTCGGGAGACTGAGACAGGAGGATCGCTTGGGCCTGGG
AGGTCGAGGCTGCAGTGAGCCAAGATTATGCCACTGCATTCCAGCCTGGGTGACAGGGCA
AGACCCTGTCTTAAAAAAAAAAAAAAAAAAAAAAAAAAAAA
>gi|21707322|gb|AAH33864.1|AAH33864 320 aa linear Similar to
branched chain aminotransferase 1, cytosolic [Homo sapiens].
MDCSNGCSAECTGEGGSKEVVGTFKAKDLIVTPATILKEKPDPNNLVFGTVFTDHMLTVE
WSSEFGWEKPHIKPLQNLSLHPGSSALHYAVELFEGLKAFRGVDNKIRLFQPNLNMDRMY
RSAVRATLPVFDKEELLECIQQLVKLDQEWVPYSTSASLYIRPTFIGTEPSLGVKKPTKA
LLFVLLSPVGPYFSSGTFNPVSLWANPKYVRAWKGGTGDCKMGGNYGSSLFAQCEAVDNG
CQQVLWLYGEDHQITEVGTMNLFLYWINEDGEEELATPPLDGIILPGVTRRCILDLAHQW
DTELSLFSINLPDFLQFIYF
>gi|29570794|gb|NM_001895.2|CSNK2A1 2323 bp mRNA Homo
sapiens casein kinase 2, alpha 1 polypeptide (CSNK2A1),
transcript variant 2, mRNA.
CCCGCCTCCTGGTAGGAGGGGGTTTCCGCTTCCGGCAGCAGCGGCTGCAGCCTCGCTCTG
GTCCCTGCGGCTGGCGGCCGAGCCGTGTGTCTCCTCCTCCATCGCCGCCATATTGTCTGT
GTGAGCAGAGGGGAGAGCGGCCGCCGCCGCTGCCGCTTCCACCACAGTTTGAAGAAAACA
GGTCTGAAACAAGGTCTTACCCCCAGCTGCTTCTGAACACAGTGACTGCCAGATCTCCAA
ACATCAAGTCCAGCTTTGTCCGCCAACCTGTCTGACATGTCGGGACCCGTGCCAAGCAGG
GCCAGAGTTTACACAGATGTTAATACACACAGACCTCGAGAATACTGGGATTACGAGTCA
CATGTGGTGGAATGGGGAAATCAAGATGACTACCAGCTGGTTCGAAAATTAGGCCGAGGT
AAATACAGTGAAGTATTTGAAGCCATCAACATCACAAATAATGAAAAAGTTGTTGTTAAA
ATTCTCAAGCCAGTAAAAAAGAAGAAAATTAAGCGTGAAATAAAGATTTTGGAGAATTTG
AGAGGAGGTCCCAACATCATCACACTGGCAGACATTGTAAAAGACCCTGTGTCACGAACC
CCCGCCTTGGTTTTTGAACACGTAAACAACACAGACTTCAAGCAATTGTACCAGACGTTA
ACAGACTATGATATTCGATTTTACATGTATGAGATTCTGAAGGCCCTGGATTATTGTCAC
AGCATGGGAATTATGCACAGAGATGTCAAGCCCCATAATGTCATGATTGATCATGAGCAC
AGAAAGCTACGACTAATAGACTGGGGTTTGGCTGAGTTTTATCATCCTGGCCAAGAATAT
AATGTCCGAGTTGCTTCCCGATACTTCAAAGGTCCTGAGCTACTTGTAGACTATCAGATG
TACGATTATAGTTTGGATATGTGGAGTTTGGGTTGTATGCTGGCAAGTATGATCTTTCGG
AAGGAGCCATTTTTCCATGGACATGACAATTATGATCAGTTGGTGAGGATAGCCAAGGTT
CTGGGGACAGAAGATTTATATGACTATATTGACAAATACAACATTGAATTAGATCCACGT
TTCAATGATATCTTGGGCAGACACTCTCGAAAGCGATGGGAACGCTTTGTCCACAGTGAA
AATCAGCACCTTGTCAGCCCTGAGGCCTTGGATTTCCTGGACAAACTGCTGCGATATGAC
CACCAGTCACGGCTTACTGCAAGAGAGGCAATGGAGCACCCCTATTTCTACACTGTTGTG
AAGGACCAGGCTCGAATGGGTTCATCTAGCATGCCAGGGGGCAGTACGCCCGTCAGCAGC
GCCAATATGATGTCAGGGATTTCTTCAGTGCCAACCCCTTCACCCCTTGGACCTCTGGCA
GGCTCACCAGTGATTGCTGCTGCCAACCCCCTTGGGATGCCTGTTCCAGCTGCCGCTGGC
GCTCAGCAGTAACGGCCCTATCTGTCTCCTGATGCCTGAGCAGAGGTGGGGGAGTCCACC
CTCTCCTTGATGCAGCTTGCGCCTGGCGGGGAGGGGTGAAACACTTCAGAAGCACCGTGT
CTGAACCGTTGCTTGTGGATTTATAGTAGTTCAGTCATAAAAAAAAAATTATAATAGGCT
GATTTTCTTTTTTCTTTTTTTTTTTAACTCGAACTTTTCATAACTCAGGGGATTCCCTGA
AAAATTACCTGCAGGTGGAATATTTCATGGACAAATTTTTTTTTCTCCCCTCCCAAATTT
AGTTCCTCATCACAAAAGAACAAAGATAAACCAGCCTCAATCCCGGCTGCTGCATTTAGG
TGGAGACTTCTTCCCATTCCCACCATTGTTCCTCCACCGTCCCACACTTTAGGGGGTTGG
TATCTCGTGCTCTTCTCCAGAGATTACAAAAATGTAGCTTCTCAGGGGAGGCAGGAAGAA
AGGAAGGAAGGAAAGAAGGAAGGGAGGACCCAATCTATAGGAGCAGTGGACTGCTTGCTG
GTCGCTTACATCACTTTACTCCATAAGCGCTTCAGTGGGGTTATCCTAGTGGCTCTTGTG
GAAGTGTGTCTTAGTTACATCAAGATGTTGAAAATCTACCCAAAATGCAGACAGATACTA
AAAACTTCTGTTCAGTAAGAATCATGTCTTACTGATCTAACCCTAAATCCAACTCATTTA
TACTTTTATTTTTAGTTCAGTTTAAAATGTTGATACCTTCCCTCCCAGGCTCCTTACCTT
GGTCTTTTCCCTGTTCATCTCCCAACATGCTGTGCTCCATAGCTGGTAGGAGAGGGAAGG
CAAAATCTTTCTTAGTTTTCTTTGTCTTGGCCATTTTGAATTC
>gi|4503095|gb|NP_001886.1|CSNK2A1 391 aa linear casein
kinase II alpha 1 subunit isoform a; CK2 catalytic subunit
alpha [Homo sapiens].
MSGPVPSRARVYTDVNTHRPREYWDYESHVVEWGNQDDYQLVRKLGRGKYSEVFEAINIT
NNEKVVVKILKPVKKKKIKREIKILENLRGGPNIITLADIVKDPVSRTPALVFEHVNNTD
FKQLYQTLTDYDIRFYMYEILKALDYCHSMGIMHRDVKPHNVMIDHEHRKLRLIDWGLAE
FYHPGQEYNVRVASRYFKGPELLVDYQMYDYSLDMWSLGCMLASMIFRKEPFFHGHDNYD
QLVRIAKVLGTEDLYDYIDKYNIELDPRFNDILGRHSRKRWERFVHSENQHLVSPEALDF
LDKLLRYDHQSRLTAREAMEHPYFYTVVKDQARMGSSSMPGGSTPVSSANMMSGISSVPT
PSPLGPLAGSPVIAAANPLGMPVPAAAGAQQ
>gi|13375963|gb|NM_024689.1|FLJ14103 2502 bp mRNA Homo
sapiens hypothetical protein FLJ14103 (FLJ14103), mRNA.
CTCTTTGGCCAAGCCCTGCCTCTGTACAGCCTCGAGTGGACAGCCAGAGGCTGCAGCTGG
AGCCCAGAGCCCAAGATGGAGCCCCAGCTGGGGCCTGAGGCTGCCGCCCTCCGCCCTGGC
TGGCTGGCCCTGCTGCTGTGGGTCTCAGCCCTGAGCTGTTCTTTCTCCTTGCCAGCTTCT
TCCCTTTCTTCTCTGGTGCCCCAAGTCAGAACCAGCTACAATTTTGGAAGGACTTTCCTC
GGTCTTGATAAATGCAATGCCTGCATCGGGACATCTATTTGCAAGAAGTTCTTTAAAGAA
GAAATAAGATCTGACAACTGGCTGGCTTCCCACCTTGGACTGCCTCCCGATTCCTTGCTT
TCTTATCCTGCAAATTACTCAGATGATTCCAAAATCTGGCGCCCTGTGGAGATCTTTAGA
CTGGTCAGCAAATATCAAAACGAGATCTCAGACAGGAAAATCTGTGCCTCTGCATCAGCC
CCAAAGACCTGCAGCATTGAGCGTGTCCTGCGGAAAACAGAGAGGTTCCAGAAATGGCTG
CAGGCCAAGCGCCTCACGCCGGACCTGGTGCAGGACTGTCACCAGGGCCAGAGAGAACTA
AAGTTCCTGTGTATGCTGAGATAACACCAGTGAAAAAGCCTGGCATGGAGCCCAGCACTG
AGAACTTCCAGAAAGTGTTAGCCTTCTCCCAACTGTGTTATACCAACCACATTTTCAAAT
AGTAATCATTAAAGAGGCTTCTGCATCAAACCTTCACATGCAGCTCCCATGCCACCCTCC
AGAATTCACCAACACACAGGCCCACCAGCAACAGGCTACCTTTGCACAATATTCTCTGAT
GACAACTCCAAAGCCCCGGCTCTTTCCACCACACTGTGGTCCCCTAGATGGGGCTGTTGC
TGAGCCCACCCCAATCCAGATGTGATCCCCCTGTGATCTACTTCTGGCAAGATTCTCAGT
CTGGACAGGTCTTCCCTATGAGATAGAACCTGATAAGGAGCTAGGGCAATTCTGACAACA
TTACCAAAGGCCCACATAACTTCTAAATTTTGGTCTGGTCTGAAGGAAAACCTGTTCTCG
CCCTAGTGATGGATGAACTCTCTTATCTCTGGCTTCTAGAGGGAAAAAAAAAGCATACCT
CTTTTACTTTTTAAGTACCTCCATCAGAGTCATGAAATCACCTGTCAAGACTATCTATCT
TTTATGTTTCCATTCTGGTAAGAACTCTTTAAATGAGGACACTGCTGATTGCTGGTGATG
TTTTTTGAGCAAACACTCGGGGGTATGGATGAAAGCCAATCGCAGGTCAAATGACTCCTT
GGGGAAGCTACTTCTCCTCTATTCAGATTTCACTAAAATCTTCCAAGATGAAAGCAAATC
TAGATTTCGGTCTTCATTGCTGTCCATTTTTGTAATGAACGAGTGTTTTTCCTTTAGCTA
GTGTATCAGGCAGGGTTCTACCAGAGAAACAGAACCAGTAGGAGATACATATACATGTCC
AGATTTATTTCAAAGAATTGATTTACATGATTGTGGGGATTGGCAAGTCCAAAATCCATA
TGGTAGGCCTGCAATCTGTAAACCTTTGGGCAGGAGCTGATGCTGTAGTTTGCAGATAGA
ATTCCTTGTTCCTTAAAAAAATCTGTTTTTGTTCTTAAGGGCTTTGAATGATTGGATCAG
GCCCACCCAGATTACCTAGATAATCTCTTTTACTTAAAGTAAACTGATTGTAGGTGCTAA
TCACATCTATGAAATGCCTTCACAGCAACACCTAGATTAGCATTCAATTGAATAACTGGG
GAATACAGCCTAGCCAAGTTGACACATAAAATTAACCATCACAGCAACATGCCTGCTAAA
TTTTATCGACCGTCTTCAGACTGTTAAGGATTGTGGTAGAGAACTGTGACAGCCACTCTC
AGCATCACCCTGAACCAAAGGCCCCTATCAAGTAACAATATAGCCAAGCAAAATTCCAGT
CAATAGAGACATTGACTGGTTGGCTGGCTTCCCAAGGGATAGCACCAGACAAGAAATGCA
AGGATGAGGAAACCAGGCACGGGAGAGGGAGGGGCAACAGAGGTCCAGGGTTTGGTTATC
TTTTTATTTTTCACTGGGAGGTGGTAAGTTAGCCCTGTTGCCCATGTATGCAGATGGGAG
AAGTGATTTAGAAACTCCAAAGCAATTGGTAATCCCCAAAATGGGTGTATCTGGTTTGAA
ATGAAACCTTATTTTATTGGAAATGGTTGGTTTCCCAATTCTGTTTGCCATTGGCCAATA
TAATTGTGGGTTTGCACATGGCCAGCACATGCCAAACAGAAGTAGACAAAGGTCTCACTC
TGTAAGTGGGACCTTGGGGAGGAGCTGCCTCCATCATAAAGGGAGGGGTTAGTAAAAATG
GTCTCTTAAGCCTGTTCCTGCTACAGTTATAGAGGTTGCTCAGAACCTTCTCAGCAAATA
TAGCAGTTATCTATTGTTGTGTATTAAACCATTTCAACACAT
>gi|13375964|gb|NP_078965.1|FLJ14103 182 aa linear hypo-
thetical protein FLJ14103 [Homo sapiens].
MEPQLGPEAAALRPGWLALLLWVSALSCSFSLPASSLSSLVPQVRTSYNFGRTFLGLDKC
NACIGTSICKKFFKEEIRSDNWLASHLGLPPDSLLSYPANYSDDSKIWRPVEIFRLVSKY
QNEISDRKICASASAPKTCSIERVLRKTERFQKWLQAKRLTPDLVQDCHQGQRELKFLCM
LR
>gi|7658290|gb|AF221842.1|AF221842 3057 bp mRNA Homo sapiens
U5 snRNP-associated 102 kDa protein mRNA., complete cds.
ACTTTGCTACGGAGTGCATCGGACGTCGAAGCCTAGAGTCTCTGCGTCTTTCCCTCTTCC
GCTGCCTCATTCCTTTCCTTCCTAGCCTTGGTCGTCGCCGCCACCATGAACAAGAAGAAG
AAACCGTTCCTAGGGATGCCCGCGCCCCTCGGCTACGTGCCGGGGCTGGGCCGGGGCGCC
ACTGGCTTCACCACGCGGTCAGACATTGGGCCCGCCCGTGATGCAAATGACCCTGTGGAT
GATCGCCATGCACCCCCAGGCAAGAGAACCGTTGGGGACCAGATGAAGAAAAATCAGGCT
GCTGACGATGACGACGAGGATCTAAATGACACCAATTACGATGAGTTTAATGGCTATGCT
GGGAGCCTCTTCTCAAGTGGACCCTACGAGAAAGATGATGAGGAAGCAGATGCTATCTAT
GCAGCCCTGGATAAAAGGATGGATGAAAGAAGAAAAGAAAGACGGGAGCAAAGGGAGAAA
GAAGAAATAGAGAAATATCGTATGGAACGCCCCAAAATCCAACAGCAGTTCTCAGACCTC
AAGAGGAAGTTGGCAGAAGTCACAGAAGAAGAGTGGCTGAGCATCCCCGAGGTTGGCGAT
GCCAGAAATAAACGTCAGCGGAACCCACGCTATGAGAAGCTGACCCCTGTTCCTGACAGT
TTCTTTGCCAAACATTTACAGACCGGAGAGAACCATACCTCAGTGGATCCCCGACAAACT
CAATTTGGAGGTCTTAACACACCCTATCCAGGTGGACTAAACACTCCATACCCAGGTGGA
ATGACGCCAGGACTGATGACACCTGGCACAGGTGAGCTGGACATGAGGAAGATTGGCCAA
GCGAGGAACACTCTGATGGACATGAGGCTGAGCCAGGTGTCTGACTCCGTGAGTGGACAG
ACCGTCGTTGACCCCAAAGGCTACCTGACGGATTTAAATTCCATGATCCCGACACACGGA
GGAGACATCAATGATATCAAGAAGGCGCGACTGCTCCTCAAGTCTGTTCGGGAGACGAAC
CCTCATCACCCGCCAGCCTGGATTGCATCAGCCCGCCTGGAAGAAGTCACTGGGAAGCTA
CAAGTAGCTCGGAACCTTATCATGAAGGGGACGGAGATGTGCCCCAAGAGTGAAGATGTC
TGGCTGGAAGCAGCCAGGTTGCAGCCTGGGGACACAGCCAAGGCCGTGGTAGCCCAAGCT
GTCCGTCATCTCCCACAGTCTGTCAGGATTTACATCAGAGCCGCAGAGCTGGAAACGGAC
ATTCGTGCAAAGAAGCGGGTTCTTCGGAAAGCCCTCGAGCATGTTCCAAACTCGGTTCGC
TTGTGGAAAGCAGCCGTTGAGCTGGAAGAACCTGAAGATGCTAGAATCATGCTGAGCCGA
GCTGTGGAGTGCTGCCCCACCAGCGTGGAGCTCTGGCTTGCTCTGGCAAGGCTGGAGACC
TATGAAAATGCCCGCAAGGTCTTGAACAAGGCGCGGGAGAACATTCCTACAGACCGACAT
ATCTGGATCACGGCTGCTAAGCTGGAGGAAGCCAATGGGAACACGCAGATGGTGGAGAAG
ATCATCGACCGAGCCATCACCTCGCTGCGGGCCAACGGTGTGGAGATCAACCGTGAGCAG
TGGATCCAGGATGCCGAGGAATGTGACAGGGCTGGGAGTGTGGCCACCTGCCAGGCCGTC
ATGCGTGCCGTGATTGGGATTGGGATTGAGGAGGAAGATCGGAAGCATACCTGGATGGAG
GATGCTGACAGTTGTGTAGCCCACAATGCCCTGGAGTGTGCACGAGCCATCTACGCCTAC
GCCCTGCAGGTGTTCCCCAGCAAGAAGAGTGTGTGGCTGCGCGCCGCGTACTTCGAGAAG
AACCATGGCACTCGGGAGTCCCTGGAAGCACTCCTGCAGAGGGCTGTGGCCCACTGCCCC
AAAGCAGAGGTGCTGTGGCTCATGGGCGCCAAGTCCAAGTGGCTGGCAGGGGATGTGCCT
GCAGCAAGGAGCATCCTGGCCCTGGCCTTCCAGGCCAACCCCAACAGTGAGGAGATCTGG
CTGGCAGCCGTGAAGCTGGAGTCCGAGAATGATGAGTACGAGCGGGCCCGGAGGCTGCTG
GCCAAGGCGCGGAGCAGTGCCCCCACCGCCCGGGTGTTCATGAAGTCTGTGAAGCTGGAG
TGGGTGCAAGACAACATCAGGGCAGCCCAAGATCTGTGCGAGGAGGCCCTGCGGCACTAT
GAGGACTTCCCCAAGCTGTGGATGATGAAGGGGCAGATCGAGGAGCAGAAGGAGATGATG
GAGAAGGCGCGGGAAGCCTATAACCAGGGGTTGAAGAAGTGTCCCCACTCCACACCCCTG
TGGCTTTTGCTCTCTCGGCTGGAGGAGAAGATTGGGCAGCTTACTCGAGCACGGGCCATT
TTGGAAAAGTCTCGTCTGAAGAACCCAAAGAACCCTGGGCTGTGGTTGGAGTCCGTGCGG
CTGGAGTACCGTGCGGGGCTGAAGAACATCGCAAATACACTCATGGCCAAGGCGCTGCAG
GAGTGCCCCAACTCCGGTATCCTGTGGTCTGAGGCCATCTTCCTCGAGGCAAGGCCCCAG
AGGAGGACCAAGAGCGTGGATGCCCTGAAGAAGTGTGAGCATGACCCCCATGTGCTCCTG
GCCGTGGCCAAGCTGTTTTGGAGTCAGCGGAAGATCACCAAGGCCAGGGAGTGGTTCCAC
CGCACTGTGAAGATTGACTCGGACCTGGGGGATGCCTGGGCCTTCTTCTACAAGTTTGAG
CTGCAGCATGGCACTGAGGAGCAGCAGGAGGAGGTGAGGAAGCGCTGTGAGAGTGCAGAG
CCTCGGCATGGGGAGCTGTGGTGCGCCGTGTCCAAGGACATCGCCAACTGGCAGAAGAAG
ATCGGGGACATCCTTAGGCTGGTGGCCGGCCGCATCAAGAACACCTTCTGATTGAGCGGT
TGCCATGGCCGGTCTCCGTGGGGCAGGGTTGGGCCGCATGTGGAAGGGCTCTGAGCTGTG
TCCTCCTTCATTAAAAGTTTTTATGTCTCGTGTCAGAAAAAAAAAAAAAAAAAAAAA
>gi|7658291|gb|AAF66128.1|AAF66128 941 aa linear U5 snRNP-
associated 102 kDa protein [Homo sapiens].
MNKKKKPFLGMPAPLGYVPGLGRGATGFTTRSDIGPARDANDPVDDRHAPPGKRTVGDQM
KKNQAADDDDEDLNDTNYDEFNGYAGSLFSSGPYEKDDEEADAIYAALDKRNDERRKERR
EQREKEEIEKYRMERPKIQQQFSDLKRKLAEVTEEEWLSIPEVGDARNKRQRNPRYEKLT
PVPDSFFAKHLQTGENHTSVDPRQTQFGGLNTPYPGGLNTPYPGGMTPGLMTPGTGELDM
RKIGQARNTLMDMRLSQVSDSVSGQTVVDPKGYLTDLNSMIPTHGGDINDIKKARLLLKS
VRETNPHHPPAWIASARLEEVTGKLQVARNLIMKGTEMCPKSEDVWLEAARLQPGDTAKA
VVAQAVRHLPQSVRIYIRAAELETDIRAKKRVLRKALEHVPNSVRLWKAAVELEEPEDAR
IMLSRAVECCPTSVELWLALARLETYENARKVLNKARENIPTDRHIWITAAKLEEANGNT
QMVEKIIDRAITSLRANGVEINREQWIQDAEECDRAGSVATCQAVMRAVIGIGIEEEDRK
HTWMEDADSCVAHNALECARAIYAYALQVFPSKKSVWLRAAYFEKNRGTRESLEALLQRA
VAHCPKAEVLWLMGAKSKWLAGDVPAARSILALAFQANPNSEEIWLAAVKLESENDEYER
ARRLLAKARSSAPTARVFMKSVKLEWVQDNIRAAQDLCEEALRHYEDFPKLWMMKGQIEE
QKEMMEKAREAYNQGLKKCPHSTPLWLLLSRLEEKIGQLTRARAILEKSRLKNPKNPGLW
LESVRLEYRAGLKNIANTLMAKALQECPNSGILWSEAIFLEARPQRRTKSVDALKKCEHD
PHVLLAVAKLFWSQRKITKAREWFHRTVKIDSDLGDAWAFFYKFELQHGTEEQQEEVRKR
CESAEPRHGELWCAVSKDIANWQKKIGDILRLVAGRIKNTF
>gi|5454165|gb|NM_006370.1|VTI1B 1287 bp mRNA Homo sapiens
vesicle transport through interaction with t-SNAREs homolog
1B (yeast) (VTI1B), mRNA.
CCCTTTCGCTGCGGCCTTTCCCCAACCCGGACCCGGCACTTCTCGGGTTCCGCGACTGCC
GATCGCCCCGGCGCGGCACCGCTCCCTCAGGAGTCGCCTAGGCCGCGCAGTCTCCCGACT
TCTCGTCAGGCTTTCGCGCCGGCGCTCCAGCAATCACTGGCTGGAGAAGGTGGGCGTTCC
GGCTCGAGAGGACCCTGCCGCGGCTCCGGAAGAGCCTCGTCCTGGGCGGCGGTGGTGCGG
CGGTCGCCGTTATGGCCACTGGGCTGGGCGGCTGACCGCGGGCTAGGAAAGGGCCCAGGG
CCCGAATCTCGGTGGCCGCTGCTCCAGCGCGGCCTGCGCCATGGCCTCCTCCGCCGCCTC
CTCGGAGCATTTCGAGAAGCTGCACGAGATCTTCCGCGGCCTCCATGAAGACCTACAAGG
GGTGCCCGAGCGGCTGCTGGGGACGGCGGGGACCGAAGAAAAGAAGAAATTGATCAGGGA
TTTTGATGAAAAGCAACAGGAAGCAAATGAAACGCTGGCAGAGATGGAGGAGGAGCTACG
TTATGCACCCCTGTCTTTCCGAAACCCCATGATGTCTAAGCTTCGAAACTACCGGAAGGA
CCTTGCTAAACTCCATCGGGAGGTGAGAAGCACACCTTTGACAGCCACACCTGGAGGCCG
AGGAGACATGAAATATGGCATATATGCTGTAGAGAATGAGCATATGAATCGGCTACAGTC
TCAAAGGGCAATGCTTCTGCAGGGCACTGAAAGCCTGAACCGGGCCACCCAAAGTATTGA
ACGTTCTCATCGGATTGCCACAGAGACTGACCAGATTGGCTCAGAAATCATAGAAGAGCT
GGGGGAACAACGAGACCAGTTAGAACGTACCAAGAGTAGACTGGTAAACACAAGTGAAAA
CTTGAGCAAAAGTCGGAAGATTCTCCGTTCAATGTCCAGAAAAGTGACAACCAACAAGCT
GCTGCTTTCCATTATCATCTTACTGGAGCTCGCCATCCTGGGAGGCCTGGTTTACTACAA
ATTCTTTCGCAGCCATTGAACTTCTATAGGGAAGGGTTTGTGGACCAGAACTTTGACCTT
GTGAATGCATGATGTTAGGGATGTGGATAGAATAAGCATATTGCTGCTGTGGGCTGACAG
TTCAAGGATGCACTGTATAGCCAGGCTGTGGGAGGAGGGAGGAAAGATGAAAAACCACTT
AAATGTGAAGGAACAACAGCAACAAGACCAGTATGATATACCAAGGTAATAAATGCTGTT
TATGACTTCTTTAAAAAAAAAAAAAAA
>gi|5454166|gb|NP_006361.1|VTI1B 232 aa linear vesicie-
associated soluble NSF attachment protein receptor (v-SN;
vesicle-associated soluble NSF attachment protein receptor
(v-SNARE; homolog of S. cerevisiae VTI1) [Homo sapiens].
MASSAASSEHFEKLHEIFRGLHEDLQGVPERLLGTAGTEEKKKLIRDFDEKQQEANETLA
EMEEELRYAPLSFRNPMMSKLRNYRKDLAKLHREVRSTPLTATPGGRGDMKYGIYAVENE
HMNRLQSQRAMLLQGTESLNRATQSIERSHRIATETDQIGSEIIEELGEQRDQLERTKSR
LVNTSENLSKSRKILRSMSRKVTTNKLLLSIIILLELAILGGLVYYKFFRSH
>gi|7705992|gb|NM_016440.1|LOC51231 1869 bp mRNA Homo
sapiens VRK3 for vaccinia related kinase 3 (LOC51231), mRNA.
CCGAGGGTCAGGCTGCAGAAGCCCAGAATCCCACCCCAGTCCCCAAGTACAGAGGTCGCT
GTCAAGATGGAGTTTCCAACCCAGTAAATCCAAGGGCCAGACCGTGACCTCATAAAGCAT
GATCTCCTTCTGTCCAGACTGTGGCAAAAGTATCCAAGCGGCATTCAAATTCTGCCCCTA
CTGTGGAAATTCTTTGCCTGTAGAGGAGCATGTAGGGTCCCAGACCTTTGTCAATCCACA
TGTGTCATCCTTCCAAGGCTCAAAGAGAGGGCTGAACTCCAGTTTTGAAACCTCTCCTAA
GAAAGTGAAATGGTCCAGCACCGTCACCTCTCCCCGATTATCCCTCTTCTCAGATGGTGA
CAGTTCTGAGTCTGAAGATACTCTGAGTTCCTCTGAGAGATCCAAAGGCTCCGGGAGCAG
ACCCCCAACCCCCAAAAGCAGCCCTCAGAAGACCAGGAAGAGCCCTCAGGTGACCAGGGG
TAGCCCTCAGAAGACCAGCTGTAGCCCTCAGAAGACCAGGCAGAGCCCTCAGACGCTGAA
GCGGAGCCGAGTGACCACCTCACTTGAAGCTTTGCCCACAGGGACAGTGCTGACAGACAA
GAGTGGGCGACAGTGGAAGCTGAAGTCCTTCCAGACCAGGGACAACCAGGGCATTCTCTA
TGAAGCTGCACCCACCTCCACCCTCACCTGTGACTCAGGACCACAGAAGCAAAAGTTCTC
ACTCAAACTGGATGCCAAGGATGGGCGCTTGTTCAATGAGCAGAACTTCTTCCAGCGGGC
CGCCAAGCCTCTGCAAGTCAACAAGTGGAAGAAGCTGTACTCGACCCCACTGCTGGCCAT
CCCTACCTGCATGGGTTTCGGTGTTCACCAGGACAAATACAGGTTCTTGGTGTTACCCAG
CCTGGGGAGGAGCCTTCAGTCGGCCCTGGATGTCAGCCCAAAGCATGTGCTGTCAGAGAG
GTCTGTGCTGCAGGTGGCCTGCCGGCTGCTGGATGCCCTGGAGTTCCTCCATGAGAATGA
GTATGTTCATGGAAATGTGACAGCTGAAAATATCTTTGTGGATCCAGAGGACCAGAGTCA
GGTGACTTTGGCAGGCTATGGCTTCGCCTTCCGCTATTGCCCAAGTGGCAAACACGTGGC
CTACGTGGAAGGCAGCAGGAGCCCTCACGAGGGGGACCTTGAGTTCATTAGCATGGACCT
GCACAAGGGATGCGGGCCCTCCCGCCGCAGCGACCTCCAGAGCCTGGGCTACTGCATGCT
GAAGTGGCTCTACGGGTTTCTGCCATGGACAAATTGCCTTCCCAACACTGAGGACATCAT
GAAGCAAAAACAGAAGTTTGTTGATAAGCCGGGGCCCTTCGTGGGACCCTGCGGTCACTG
GATCAGGCCCTCAGAGACCCTGCAGAAGTACCTGAAGGTGGTGATGGCCCTCACGTATGA
GGAGAAGCCGCCCTACGCCATGCTGAGGAACAACCTAGAAGCTTTGCTGCAGGATCTGCG
TGTGTCTCCATATGACCCCATTGGCCTCCCGATGGTGCCCTAGGTGGAATCCAGAACTTT
CCATTTGCAGTGTGCAACAGAAAAAAAAATGAAGCAATGTGACTCAAGGCCTGCTGTTTA
ATCACAGATAAGCTTCTAGAACAAGCCCTGGAATGTGCATTCCTGCCACTGGTTTCAGGA
TACTCATCAGTCCTGATTAGCCTCCGGAGGGCCCCAGTTTCCCTCCCGTGAATGTGAAGT
TCCCCATCTTGGTGGCCTGCCCTTCAGCCAGTGTCCTAGCAAAGCTGGATGGGGTTGGGC
CGGCCCACAGGGGGGACCCCTCCTACCCTTGACTCCTCTGTGCTTTGGTAATAAATTGTT
TTACCAGAG
>gi|7705993|gb|NP_057524.1|LOC51231 474 aa linear VRK3 for
vaccinia related kinase 3 [Homo sapiens].
MISFCPDCGKSIQAAFKFCPYCGNSLPVEEHVGSQTFVNPHVSSFQGSKRGLNSSFETSP
KKVKWSSTVTSPRLSLFSDGDSSESEDTLSSSERSKGSGSRPPTPKSSPQKTRKSPQVTR
GSPQKTSCSPQKTRQSPQTLKRSRVTTSLEALPTGTVLTDKSGRQWKLKSFQTRDNQGIL
YEAAPTSTLTCDSGPQKQKFSLKLDAKDGRLFNEQNFFQRAAKPLQVNKWKKLYSTPLLA
IPTCMGFGVHQDKYRFLVLPSLGRSLQSALDVSPKHVLSERSVLQVACRLLDALEFLHEN
EYVHGNVTAENIFVDPEDQSQVTIAGYGFAFRYCPSGKHVAYVEGSRSPHEGDLEFISMD
LHKGCGPSRRSDLQSLGYCMLKWLYGFLPWTNCLPNTEDIMKQKQKFVDKPGPFVGPCGH
WIRPSETLQKYLKVVMALTYEEKPPYAMLRNNLEALLQDLRVSPYDPIGLPMVP
>gi|27479296|gb|XM_114075.2|TCEA3 1543 bp mRNA Homo sapiens
transcription elongation factor A (SII), 3 (TCEA3), mRNA.
CGCCCCCGCCGGGCGTGTGTGTCGTGTGTGTTTGGGGCCCGCGCGGGTTGCGCGCCCTCC
GCCTTCGCGCCTCCTGCCCCCGAGGCCCTACTGCTGCCCCTGTGCCCCTCGCCCCGCCGG
GCGTCGCGGGCCAACATGGGCCAGGAAGAGGAGCTGCTGAGGATCGCCAAAAAGCTGGAG
AAGATGGTGGCCAGGAAGAACACGGAAGGGGCCCTGGACCTTCTGAAGAAGCTGCACAGC
TGCCAGATGTCCATCCAGCTACTACAGACAACCAGGATTGGAGTTGCTGTTAATGGGGTC
CGCAAGCACTGCTCAGACAAGGAGGTGGTGTCCTTGGCCAAAGTCCTTATCAAAAACTGG
AAGCGGCTGCTAGACTCCCCTGGACCCCCAAAAGGAGAAAAAGGAGAGGAAAGAGAAAAG
GCAAAGAAGAAGGAAAAAGGGCTTGAGTGTTCAGACTGGAAGCCAGAAGCAGGCCTTTCT
CCACCAAGGAAAAAACGAGAAGACCCCAAAACCAGGAGAGACTCTGTGGACTCCAAGTCT
TCTGCCTCCTCCTCTCCAAAAAGACCATCGGTGGAAAGATCAAACAGCAGCAAATCAAAA
GCGGAGAGCCCCAAAACACCTAGCAGCCCCTTGACCCCCACGTTTGCCTCTTCCATGTGT
CTCCTGGCCCCCTGCTATCTCACAGGGGACTCTGTCCGGGACAAGTGTGTGGAGATGCTG
TCAGCAGCCCTGAAGGCGGACGATGATTACAAGGACTATGGAGTCAACTGTGACAAGATG
GCATCAGAATCGAAAGATCATATCTACCAAGAGCTCAAGAGCACGGACATGAAGTACCGG
AACCGCGTGCGCAGCCGCATAAGCAACCTCAAGGACCCCAGGAACCCCGGCCTGCGGCGG
AACGTGCTCAGTGGGGCCATCTCCGCAGGGCTTATAGCCAAGATGACGGCAGAGGAAATG
GCCAGTGATGAACTGAGGGAGTTGAGGAATGCCATGACCCAGGAGGCCATCCGTGAGCAC
CAGATGGCCAAGACTGGCGGCACCACCACTGACCTCTTCCAGTGCAGCAAATGCAAGAAG
AAGAACTGCACCTATAACCAGGTGCAGACACGCAGTGCTGATGAGCCCATGACTACCTTT
GTCTTATGCAATGAATGTGGCAATCGCTGGAAGTTCTGCTGATGGAACAGCCAGCCATGA
ACAAGGTGAGGAAGAAGAAAGAGGAAGCGCTGAATTATCTGAACTGGAGAAGCAATAAAA
ATTAAAGTGAAGGAAAATACTGAACTCTGTCTGAGTGGGATGGTATGAGTTAGAGGAAGA
ATTCTCTTGCAAATTAATAATCGGTCATTAGAAACAATTGGTTAATGGGGGAGCCTAATT
GGAGAATGATGCTGAGAATTTGTATTGATGAACCTCTTTTAGAAACTGCAGAGGGCTGGG
CACGGTGGTTTATGGCTGTAATCTGCAAACTCTGGGAGGCTGAGGTGGGAGAATCGCTTA
ACCCCAGAAGTTTGAGTCCAGCCCAGGCAACACAGCAAGACCC
>gi|20473950|gb|XP_114075.1|TCEA3 348 aa linear similar to
Transcription elongation factor A protein 3 (Transcription
elongation factor S-II protein 3) (Transcription elongation
factor TFIIS.h) [Homo sapiens].
MGQEEELLRIAKKLEKMVARKNTEGALDLLKKLHSCQMSIQLLQTTRIGVAVNGVRKHCS
DKEVVSLAKVLIKNWKRLLDSPGPPKGEKGEEREKAKKKEKGLECSDWKPEAGLSPPRKK
REDPKTRRDSVDSKSSASSSPKRPSVERSNSSKSKAESPKTPSSPLTPTFASSMCLLAPC
YLTGDSVRDKCVEMLSAALKADDDUKDUGVNCDKMASEIEDHIYQELKSTDMKYRNRVRS
RISNLKDPRNPGLRRNVLSGAISAGLIAKMTAEEMASDELRELRNAMTQEAIREHQMAKT
GGTTTDLFQCSKCKKKNCTYNQVQTRSADEPMTTFVLCNECGNRWKFC
>gi|21314607|gb|NM_003342.2|UBE2G1 2430 bp mRNA Homo sapiens
ubiquitin-conjugating enzyme E2G 1 (UBC7 homolog, C.
elegans) (UBE2G1), mRNA.
ACCGGCAGCGAGGCGCCGCTCCCGCCGCCTCAGCCCGGCCTTCCTCGGCTCCGGCGCTCC
GGTCGCGGGGCCCGGGTTCCTCGGCACACCCCGCTCCAGCCGCCCCCAGAGCCTGTCCCC
AGCCCTTCGGAAGCCCCGGCGCCAGCCCGGGCCCTCGGCAGGGAGGATGACGGAGCTGCA
GTCGGCACTGCTACTGCGAAGACAGCTGGCAGAACTCAACAAAAATCCAGTGGAAGGCTT
TTCTGCAGGTTTAATAGATGACAATGATCTCTACCGATGGGAAGTCCTTATTATTGGCCC
TCCAGATACACTTTATGAAGGTGGTGTTTTTAAGGCTCATCTTACTTTCCCAAAAGATTA
TCCCCTCCGACCTCCTAAAATGAAATTCATTACAGAAATCTGGCACCCAAATGTTGATAA
AAATGGTGATGTGTGCATTTCTATTCTTCATGAGCCTGGGGAAGATAAGTATGGTTATGA
AAAGCCAGAGGAACGCTGGCTCCCTATCCACACTGTGGAAACCATCATGATTAGTGTCAT
TTCTATGCTGGCAGACCCTAATGGAGACTCACCTGCTAATGTTGATGCTGCGAAAGAATG
GAGGGAAGATAGAAATGGAGAATTTAAAAGAAAAGTTGCCCGCTGTGTAAGAAAAAGCCA
AGAGACTGCTTTTGAGTGACATTTATTTAGCAGCTAGTAACTTCACTTATTTCAGGGTCT
CCAATTGAGAAACATGGCACTGTTTTTCCTGCACTCTACCCACCTATTGCTGGACTTCTG
TTGTACAAGTTGGCAAACACTGGCTGGAACTGGGCTGCAATAAAACATGCCAGTTATCAA
TGCTGACAAGAGCCTAACAAGTGCCAACTTACAGATGATTACGCATTTTGAATTCTAATG
AACTGTTTTAACCTTCAGGAAGAATTGTAAAGACCTGTACATAGCACAACATGATCCGGA
TAATATATATACTGTTCATGTACATCCACAAATACACCTTGTACCAAATAATGCTTTCTT
GTAGTAGAATAAGAATCGTGTAAATTCTAAGAGATTTTAGCAGGTTTTCTTTCCTATTCA
TTGTTTCTTATCAGTTTAAAAGGATTCCTTTAAGCATGTCAGATGAAAAGCAATTAGGAT
TAAAAGTTTCCATTTAATTTCCCTTAAACCCTTGAGGCTTCATTAAACTCTTTTCACTTA
CTAAACTTTTGTATCTTCTTTGTTTTGACACACTCCCCTTTGCTTTTATCTCTTACCTGC
CAGAATGTTCTCAAATGATTTAGTTCAAATACTGAAATACTTAATGAGCAATTACTTGAT
TTTTAATGATGACTTCGAAGGAGTCATCACTAGGTGCTTTGTCCTTTTTGTATTCTAGTT
GCACCCACCTCTTGGATTGGATATAGCAATAACATTTATTGGCCGTTGTGAGCTCTTGAT
CCCAGTCATTACCCCTGAGAACTAAAAATAGATGGTTCTTAATTCAACTTACTGAAAATT
TCCCCAAACAATAGCAAATCTGACTTTTCCCTCTTCAGTTGCCTGGTATTAAGGTTGGAT
AAATGAAGCATGCACAGCTACAGGCTTTCTACTTAACTTCTGGGTTTGCTATTACAAATC
CTATTTACTCTCATACCCTTCTCCTTAGTCCTTCATATTTCTCTGCCTCTATTCTTCTAT
ACTGCAGATTTTTCTCACCTATTGTACAAAGAAATTGCGATGTATATTTTCATGTAATTT
GATTTTGGAATTCTGTCACCTTATGTAGTGAGTTCTTCCAAAATATAATTTTTTTTCAAT
AATTGTCAAGTTGTTGGCTTTTATTGTATTGAATGAAGGCTATAATACTGAGTGCCAGAG
AAGTGGTTTAGGAAAATCTCAGGTTGATTCCTTATGCAAATGAACTTTTAATACTTGAAA
ATCACATGGCCATGGCAGTATATGTATTTGGTTCTATCTAGATTCTTCTGTGAATCTAAA
AGCATTACAGGGGTAAATGCTTTGCTATTTGACGTATAGATCCCGTCACTAACAATAGTA
CACTTGGATGTGATTAATGTTTGAGCTTCAATATATTTCATATCATACAGTTTTCTAAAA
CAACTTCAGCAAATGGTAAAATGAACATGTGCAGTGTTAAAGGCAGGCCTTAGGCTCCTT
CATGTTTGTTGTGAGGTTGTGTGTGGGAAGTAGTCTTTGGCTTATAAGGGATAGAACTTG
AGACAGTAGCAGATGGGACATGGTGTTTGATTGTGAGAATCAGTGAGAATTCGTGCATCT
CTGCTCTGTGGGGTTTGGAGAAATGCTTTGGCAGAAGAGTGAAAGAACTCCTGCCAAGAG
CCCAGACCTCTACAAACGTTGTATGTCCTTTTTTAAGCAGAAATAAAATGGTTGAGGACG
AAAAAAAAAAAAAAAAAAAAAAAAAAAAAA
>gi|13489085|gb|NP_003333.1|UBE2G1 170 aa linear ubiquitin-
conjugating enzyme E2G 1 (UBC7 homolog, C. elegans);
Ubiquitin-conjugating enzyme E2G (UBC7, C. elegans, human
homolog of); ubiquitin-conjugating enzyme E2G (homologous to
C. elegans UBC7); ubiquitin-conjugating enzyme E2G 1
(homologous to C. elegans UBC7) [Homo sapiens].
MTELQSALLLRRQLAELNKNPVEGFSAGLIDDNDLYRWEVLIIGPPDTLYEGGVFKAHLT
FPKDYPLRPPKMKFITEIWHPNVDKNGDVCISILHEPGEDKYGYEKPEERWLPIHTVETI
MISVISMLADPNGDSPANVDAAKEWREDRNGEFKRKVARCVRKSQETAFE
>gi|21361498|gb|NM_015670.2|SENP3 2258 bp mRNA Homo sapiens
sentrin/SUMO-specific protease 3 (SENP3), mRNA.
GAAGCTTGAGGCCGGAGACGCCCGCCTTCGGGCCCGTCCGCCCGGCTTCCCCGCTCCCGG
GTACTGGAAGATGAAAGAGACTATACAAGGGACCGGGTCCTGGGGGCCTGAGCCTCCTGG
ACCCGGCATACCCCCAGCTTACTCAAGTCCCAGGCGGGAGCGTCTTCGTTGGCCCCCACC
TCCCAAACCCCGACTCAAGTCAGGTGGAGGGTTTGGGCCAGATCCTGGGTCAGGGACCAC
AGTGCCAGCCAGACGCCTCCCTGTCCCCCGACCCTCTTTTGATGCCTCAGCAAGTGAAGA
GGAGGAAGAAGAGGAGGAGGAGGAGGATGAAGATGAAGAGGAGGAAGTGGCAGCTTGGAG
GCTGCCCCCAAGATGGAGTCAGCTGGGAACCTCCCAGCGGCCCCGCCCTTCCCGCCCCAC
TCATCGAAAAACCTGCTCACAGCGCCGCCGCCGAGCCATGAGAGCCTTCCGGATGCTGCT
CTACTCAAAAAGCACCTCGCTGACATTCCACTGGAAGCTTTGGGGGCGCCACCGGGGCCG
GCGGCGGGGCCTCGCACACCCCAAGAACCATCTTTCACCCCAGCAAGGGGGTGCGACGCC
ACAGGTGCCATCCCCCTGTTGTCGTTTTGACTCCCCCCGGGGGCCACCTCCACCCCGGCT
GGGTCTGCTAGGTGCTCTCATGGCTGAGGATGGGGTGAGAGGGTCTCCACCAGTGCCCTC
TGGGCCCCCCATGGAGGAAGATGGACTCAGGTGGACTCCAAAGTCTCCTCTGGACCCTGA
CTCGGGCCTCCTTTCATGTACTCTGCCCAACGGTTTTGGGGGACAATCTGGGCCAGAAGG
GGAGCGCAGCTTGGCACCCCCTGATGCCAGCATCCTCATCAGCAATGTGTGCAGCATCGG
GGACCATGTGGCCCAGGAGCTTTTTCAGGGCTCAGATTTGGGCATGGCAGAAGAGGCAGA
GAGGCCTGGGGAGAAAGCCGGCCAGCACAGCCCCCTGCGAGAGGAGCATGTGACCTGCGT
ACAGAGCATCTTGGACGAATTCCTTCAAACGTATGGCAGCCTCATACCCCTCAGCACTGA
TGAGGTAGTAGAGAAGCTGGAGGACATTTTCCAGCAGGAGTTTTCCACCCCTTCCAGGAA
GGGCCTGGTGTTGCAGCTGATCCAGTCTTACCAGCGGATGCCAGGCAATGCCATGGTGAG
GGGCTTCCGAGTGGCTTATAAGCGGCACGTGCTGACCATGGATGACTTGGGGACCTTGTA
TGGACAGAACTGGCTCAATGACCAGGTGATGAACATGTATGGAGACCTGGTCATGGACAC
AGTCCCTGAAAAGGTGCATTTCTTCAATAGTTTCTTCTATGATAAACTCCGTACCAAGGG
TTATGATGGGGTGAAAAGGTGGACCAAAAACGTGGACATCTTCAATAAGGAGCTACTGCT
AATCCCCATCCACCTGGAGGTGCATTGGTCCCTCATCTCTGTTGATGTGAGGCGACGCAC
CATCACCTATTTTGACTCGCAGCGTACCCTAAACCGCCGCTGCCCTAAGCATATTGCCAA
GTATCTACAGGCAGAGGCGGTAAAGAAAGACCGACTGGATTTCCACCAGGGCTGGAAAGG
TTACTTCAAAATGAATGTGGCCAGGCAGAATAATGACAGTGACTGTGGTGCTTTTGTGTT
GCAGTACTGCAAGCATCTGGCCCTGTCTCAGCCATTCAGCTTCACCCAGCAGGACATGCC
CAAACTTCGTCGGCAGATCTACAAGGAGCTGTGTCACTGCAAACTCACTGTGTGAGCCTC
GTACCCCAGACCCCAAGCCCATAAATGGGAAGGGAGACATGGGAGTCCCTTCCCAAGAAA
CTCCAGTTCCTTTCCTCTCTTGCCTCTTCCCACTCACTTCCCTTTGGTTTTTCATATTTA
AATGTTTCAATTTCTGTATTTTTTTTTCTTTGAGAGAATACTTGTTGATTTCTGATGTGC
AGGGGGTGGCTACAGAAAAGCCCCTTTCTTCCTCTGTTTGCAGGGGAGTGTGGCCCTGTG
GCCTGGGTGGAGCAGTCATCCTCCCCCTTCCCCGTGCAGGGAGCAGGAAATCAGTGCTGG
GGGTGGTGGGCGGACAATAGGATCACTGCCTGCCAGATCTTCAAACTTTTATATATATAT
ATATATATATATATATATATATAAAAATATATAAATGCCACGGTCCTGCTCTGGTCAATA
AAGGATCCTTTGTTGATACGTAAAAAAAAAAAAAAAAA
>gi|21361499|gb|NP_056485.2|SENP3 574 aa linear sentrin/
SUMO-specific protease 3 [Homo sapiens].
MKETIQGTGSWGPEPPGPGIPPAYSSPRRERLRWPPPPKPRLKSGGGFGPDPGSGTTVPA
RRLPVPRPSFDASASEEEEEEEEEEDEDEEEEVAAWRLPPRWSQLGTSQRPRPSRPTHRK
TCSQRRRRAMRAFRMLLYSKSTSLTFHWKLWGRHRGRRRGLAHPKNHLSPQQGGATPQVP
SPCCRFDSPRGPPPPRLGLLGALMAEDGVRGSPPVPSGPPMEEDGLRWTPKSPLDPDSGL
LSCTLPNGFGGQSGPEGERSLAPPDASILISNVCSIGDHVAQELFQGSDLGMAEEABRPG
EKAGQHSPLREEHVTCVQSILDEFLQTYGSLIPLSTDEVVEKLEDIFQQEFSTPSRKGLV
LQLIQSYQRMPGNAMVRGFRVAYKRHVLTMDDLGTLYGQNWLNDQVMNMYGDLVMDTVPE
KVHFFNSFFYDKLRTKGYDGVKRWTKNVDIFNKELLLIPIHLEVHWSLISVDVRRRTITY
FDSQRTLNRRCPKHIAKYLQAEAVKKDRLDFHQGWKGYFKMNVARQNNDSDCGAFVLQYC
KHLALSQPFSFTQQDMPKLRRQIYKELCHCKLTV
>gi|5803166|gb|NM_006802.1|SF3A3 2733 bp mRNA Homo sapiens
splicing factor 3a, subunit 3, 60kDa (SF3A3), mRNA.
AAGGGAAGATGGAGACAATACTGGAGCAGCAGCGGCGCTATCATGAGGAGAAGGAACGGC
TCATGGACGTCATGGCTAAAGAGATGCTCACCAAGAAGTCCACGCTCCGGGACCAGATCA
ATTCTGATCACCGCACTCGGGCCATGCAAGATAGGTATATGGAGGTCAGTGGGAACCTGA
GGGATTTGTATGATGATAAGGATGGATTACGAAAGGAGGAGCTCAATGCCATTTCAGGAC
CCAATGAGTTTGCTGAATTCTATAATAGACTCAAGCAAATAAAGGAATTCCACCGGAAGC
ACCCAAATGAGATCTGTGTGCCAATGTCAGTGGAATTTGAGGAACTCCTGAAGGCTCGAG
AGAATCCAAGTGAAGAGGCACAAAACTTGGTGGAGTTCACAGATGAGGAGGGATATGGTC
GTTATCTCGATCTCCATGACTGTTACCTCAAGTACATTAACCTGAAGGCATCTGAGAAGC
TGGATTATATCACATACCTGTCCATCTTTGACCAATTATTTGACATTCCTAAAGAAAGGA
AGAATGCAGAGTATAAGAGATACCTAGAGATGCTGCTTGAGTACCTTCAGGATTACACAG
ATAGAGTGAAGCCTCTCCAAGATCAGAATGAACTTTTTGGGAAGATTCAGGCTGAGTTTG
AGAAGAAATGGGAGAATGGGACCTTTCCTGGATGGCCGAAAGAGACAAGCAGTGCCCTGA
CCCATGCTGGAGCCCATCTTGACCTCTCTGCATTCTCCTCCTGGGAGGAGTTGGCTTCTC
TGGGTTTGGACAGATTGAAATCTGCTCTCTTAGCTTTAGGCTTGAAATGTGGCGGGACCC
TAGAAGAGCGAGCCCAGAGACTATTCAGTACCAAAGGAAAGTCCCTGGAGTCACTTGATA
CCTCTTTGTTTGCCAAAAATCCCAAGTCAAAGGGCACCAAGCGAGACACTGAAAGGAACA
AAGACATTGCTTTTCTAGAAGCCCAGATCTATGAATATGTAGAGATTCTCGGGGAACAGC
GACATCTCACTCATGAAAATGTACAGCGCAAGCAAGCCAGGACAGGAGAAGAGCGAGAAG
AAGAGGAAGAAGAGCAGATCAGTGAGAGTGAGAGTGAAGATGAAGAGAACGAGATCATTT
ACAACCCCAAAAACCTGCCACTTGGCTGGGATGGCAAACCTATTCCCTACTGGCTGTATA
AGCTTCATGGCCTAAATATCAACTACAACTGTGAGATTTGTGGAAACTACACCTACCGAG
GGCCCAAAGCCTTCCAGCGACACTTTGCTGAATGGCGTCATGCTCATGGCATGAGGTGTT
TGGGCATCCCAAATACTGCTCACTTTGCTAATGTGACACAGATTGAAGATGCTGTCTCCT
TGTGGGCCAAACTGAAATTGCAGAAGGCTTCAGAACGATGGCAGCCTGACACTGAGGAAG
AATATGAAGACTCAAGTGGGAATGTTGTGAATAAGAAGACATACGAGGATCTGAAAAGAC
AAGGACTGCTCTAGTGTTGAGGGATGTAGCTCAGCTTTTGGGCTAGCCCAGGCTTCCCTA
AGATCTGCTTTTTCTATTTCTCCCAACCAAATCCTCTTAAAGACCCTTTGCTATGTAGTC
TCATGGTCTAGCATGCATCTTGTAGAAACAAGGCATGCTGGCAGATTGCAGGGTTGAGAT
GTGTTTTATCTGTTTTATATTTTAAAAGATTCTGCCAGAAAATAAAACCAGACCTTGTTC
TAAAGCCCAGGGTTATGGACCAACTCAGTGCTTCAGGTCTTAATGCCTCCATACCTCTTC
CTCACCAACTTTACTAGTAGCTGAGATTTAATGGGCACCTATTATGCTACATATCATGTT
AGGTAAATCTGACCTGACCTCTTTCCCCACCCTCCTTTGTTGCTGCTTCCCTGAATGAGT
ATTACCCCAGGATGAGGTCTGCCATCAGCTTAGTTAGCCATTGATGCAAATACTAGGGAA
AGACTAGGAGGATGAGCCAGGGTTGCTACTAAGGACTAAGTGTCGCACCAAGGTTTGCCT
TTTGTATTTGCATAAAGAAAGGAGTTGGAGCTGGGTGCAGTGGCTTGTGCCTGTAGTCCC
AGCTACTTGGGAGGCTGAGGCAGGAGGGTTGCTTGAGACTAGCCTAGGTAACATAGTGAG
ACCCTGTCTCATTAAAAAAAAAAAAAAAAGGCATGGTGGCACGCACTGTAGTCCCAGCTA
CTCAGGAGACTGAGGCTAGAAGATCCTTTGAACCTAGGAGTTTGAGACCAGCCTGGGCGA
TATAGTGAGGCCCCATCTCAAAAAAAAAAAAAAGCGGGGGGGGGGAGTTGGGCTGTGTTG
GAATGGGCCTGCAGCCCAACAAACAAGGGAACTAGGACCGACAGTGACTTCACCAGCTTG
CTAGGTCAGAATGAGAGACTGGTGGGTCTGTCTACCTGTTTCTTCTACAAGATCCCTATT
TGACTGTAAAAGTAGCTAATACTCACATGTTCTCCAATCCCAGGTAGCCATGGTAGAGTT
GGGTAGAGTTGAGCAGCCGCCCCAGGATCCAAATGTGGTGTCTGAAATGGAAAGAACTAA
GGCAACCAGGAAGGCACTGATCTGCCTTATAAGCACAGTCATCTGAAAGTCAGGCCTGCT
GCAGGACAGGATCCCCCAGAGACCCCATTTGCCTCTCAACACTCAGACCTTCAACTGTTT
TTTAATAAATCTACTTTTTAAAAAAAAAAAATA
>gi|5803167|gb|NP_006793.1|SF3A3 501 aa linear splicing
factor 3a, subunit 3, 60kDa; pre-mRNA splicing factor SF3a
(60kD) [Homo sapiens].
METILEQQRRYHEEKERLMDVMAKEMLTKKSTLRDQINSDHRTRAMQDRYMEVSGNLRDL
YDDKDGLRKEELNAISGPNEFAEFYNRLKQIKEFHRKHPNEICVPMSVEFEELLKARENP
SEEAQNLVEFTDEEGYGRYLDLHDCYLKYINLKASEKLDYITYLSIFDQLFDIPKERKNA
EYKRYLEMLLEYLQDYTDRVKPLQDQNELFGKIQAEFEKKWENGTFPGWPKETSSALTHA
GAHLDLSAFSSWEELASLGLDRLKSALLALGLKCGGTLEERAQRLFSTKGKSLESLDTSL
FAKNPKSKGTKRDTERNKDIAFLEAQIYEYVEILGEQRHLTHENVQRKQARTGEEREEEE
EEQISESESEDEENEIIYNPKNLPLGWDGKPIPYWLYKLHGLNINYNCEICGNYTYRGPK
AFQRHFAEWRHAHGMRCLGIPNTAEFANVTQIEDAVSLWAKLKLQKASERWQPDTEEEYE
DSSGNVVNKKTYEDLKRQGLL
>gi|28882054|gb|NM_005011.2|NRF1 2514 bp mRNA Homo sapiens
nuclear respiratory factor 1 (NRF1), mRNA.
GAGGCTGCGAGGAGCCGGCGCGGTCGCAGTCTCCACGGCGCAGGCCCACGGTAGCGCAGC
CGCTCTGAGTAGAACTTCATGGAGGAACACGGAGTGACCCAAACCGAACATATGGCTACC
ATAGAAGCACATGCAGTGGCCCAGCAAGTGCAGCAGGTCCATGTGGCTACTTACACCGAG
CATAGTATGCTGAGTGCTGATGAAGACTCGCCTTCTTCTCCCGAGGACACCTCTTACGAT
GACTCAGATATACTCAACTCCACAGCAGCTGATGAGGTGACAGCTCATCTGGCAGCTGCA
GGTCCTGTGGGAATGGCCGCTGCTGCTGCTGTGGCAACAGGAAAGAAACGGAAACGGCCT
CATGTATTTGAGTCTAATCCATCTATCCGGAAGAGGCAACAAACACGTTTGCTTCGGAAA
CTTCGAGCCACGTTAGATGAATATACTACTCGTGTGGGACAGCAAGCTATTGTCCTCTGT
ATCTCACCCTCCAAACCTAACCCTGTCTTTAAAGTGTTTGGTGCAGCACCTTTGGAGAAT
GTGGTGCGTAAGTACAAGAGCATGATCCTGGAAGACCTGGAGTCTGCTCTGGCAGAACAC
GCCCCTGCGCCACAGGAGGTTAACTCAGAACTGCCGCCTCTCACCATCGACGGAATTCCA
GTCTCTGTGGACAAAATGACCCAGGCCCAGCTTCGGGCATTTATCCCAGAGATGCTCAAG
TACTCTACAGGTCGGGGAAAACCAGGCTGGGGGAAAGAAAGCTGCAAGCCCATCTGGTGG
CCTGAAGATATCCCCTGGGCAAATGTCCGGAGTGATGTCCGCACAGAAGAGCAAAAGCAG
AGGGTTTCATGGACCCAGGCACTACGGACCATAGTTAAAAACTGTTATAAACAGCATGGG
CGGGAAGACCTTTTGTATGCCTTTGAAGATCAGCAAACGCAAACACAGGCCACAGCCACA
CATAGTATAGCTCATCTTGTACCATCACAGACTGTAGTCCAGACTTTTAGTAACCCTGAT
GGCACTGTCTCACTTATCCAGGTTGGTACGGGGGCAACAGTAGCCACATTGGCTGATGCT
TCAGAATTGCCAACCACGGTCACCGTTGCCCAAGTGAATTATTCTGCCGTGGCTGATGGA
GAGGTGGAACAAAATTGGGCCACGTTACAGGGAGGTGAGATGACCATCCAGACGACGCAA
GCATCAGAGGCCACCCAGGCGGTGGCATCGTTGGCAGAGGCCGCAGTGGCAGCTTCTCAG
GAGATGCAGCAGGGAGCTACAGTCACTATGGCGCTTAACAGCGAAGCTGCCGCCCATGCT
GTCGCCACCCTGGCTGAGGCCACCTTACAAGGTGGGGGACAGATCGTCTTGTCTGGGGAA
ACCGCAGCAGCCGTCGGAGCACTTACTGGAGTCCAAGATGCTAATGGCCTCTTTATGGCA
GATCGTGCAGGTCGCAAGTGGATCCTGACTGACAAAGCCACAGGCCTGGTCCAGATCCCT
GTGAGCATGTACCAGACTGTGGTGACCAGCCTCGCCCAGGGCAACGGACCAGTGCAGGTG
GCCATGGCCCCTGTGACCACCAGGATATCAGACAGCGCAGTCACCATGGACGGCCAAGCT
GTGGAGGTGGTGACATTGGAACAGTGACATACAGCCATATTATGGCATCGTTTTCTAGTC
TACTTCAAAATTTTTTACACGTTTGCAGAGGTGCAATCAAATGGAATTAAGTCTCTCGAC
TTTGGAAGGAAAGTTTTGTTAACCTTTTTTTTTTTAAAAGGAAGAAAGCGGATTTTGGAA
TTGCATTTTTTAAAGCACCACTCTTGATTTTCTGGGATTGGTGAAGAAACTGCATTGTCA
ATTTCACTGTCCCAAAAAAGCCAAATTGTGGCAGGACTTCTTTCTGCGGAAATGTGTGTG
TATACTTATGTGTGTGTATGTGTGAGTGTGAATATATGTATATGTGTACATATGGACATA
CACATTTACATATATATAAAGTATATATATACATATATATATATATATGTATGAAACCCG
CATGGAATTATCTGTATGAAATCAAGGTGCGCTGTGGAAACAATAATTCACCCAGTTTAG
TGGGTGGTAGGGTACGTGGCCAGACACAGTCACCCAGTTTTTGTTCATACCAGGGTCATG
CGTTGAGCTACTGACAAACTCAGGCGGAGGTGACCATGCCCTTCACCAAAGCTGCCTCCC
AGTGGCCACACAGAACTCTCCCTGCTGGACTCACCTGAGGAAAGAGGCTCCAGCATGGGG
TGGGTCAGAGATGTGCTTGCAAGGTCCAGGGACTGCGTGGTCTGCCAGCTGAGATGCTCC
TCGGGCTGGCCCAGGTGCTGACCTTGCCACAGGCAGATGAATGTCTTGAAAGCTCCCGGG
CCTCAGCCTCCCATCTCCTCTCCTTCCCAGGAATCCTTGATCTCATGACTATTAAAATGT
TGCTCTGGTTTTAAGGTCAAAAAAAAAAAAAAAAAAAAAAAAAAAAAAAAAAAA
>gi|28882055|gb|NP_005002.2|NRF1 522 aa linear nuclear
respiratory factor 1 [Homo sapiens].
MEEHGVTQTEHMATIEAHAVAQQVQQVHVATYTEHSMLSADEDSPSSPEDTSYDDSDILN
STAADEVTAHLAAAGPVGMAAAAAVATGKKRKRPHVFESNPSIRKRQQTRLLRKLRATLD
EYTTRVGQQAIVLCISPSKPNPVFKVFGAAPLENVVRKYKSMILEDLESALAEHAPAPQE
VNSELPPLTIDGIPVSVDKMTQAQLRAFIPEMLKYSTGRGRPGWGKESCKPIWWPEDIPW
ANVRSDVRTEEQKQRVSWTQALRTIVKNCYKQHGREDLLYAFEDQQTQTQATATHSIAHL
VPSQTVVQTFSNPDGTVSLIQVGTGATVATLADASELPTTVTVAQVNYSAVADGEVEQNW
ATLQGGEMTIQTTQASEATQAVASIAEAAVAASQEMQQGATVTMALNSEAAAHAVATLAE
ATLQGGGQIVLSGETAAAVGALTGVQDANGLFMADRAGRKWILTDKATGLVQIPVSMYQT
VVTSLAQGNGPVQVAMAPVTTRISDSAVTMDGQAVEVVTLEQ
>gi|6996000|gb|NM_001663.2|ARF6 1806 bp mRNA Homo sapiens
ADP-ribosylation factor 6 (ARF6), mRNA.
GGCCGGAGGGAGCCCGCGCTCGGGGCGGCGGCTGGAGGCAGCGCACCGAGTTCCCGCGAG
GATCCATGACCTGACGGGGCCCCGGAGCCGCGCTGCCTCTCGGGTGTCCTGGGTCGGTGG
GGAGCCCAGTGCTCGCAGGCCGGCGGGCGGGCCGGAGGGCTGCAGTCTCCCTCGCGGTGA
GAGGAAGGCGGAGGAGCGGGAACCGCGGCGGCGCTCGCGCGGCGCCTGCGGGGGGAAGGG
CAGTTCCGGGCCGGGCCGCGCCTCAGCAGGGCGGCGGCTCCCAGCGCAGTCTCAGGGCCC
GGGTGGCGGCGGCGACTGGAGAAATCAAGTTGTGCGGTCGGTGATGCCCGAGTGAGCGGG
GGGCCTGGGCCTCTGCCCTTAGGAGGCAACTCCCACGCAGGCCGCAAAGGGCTCTCGCGG
CCGAGAGGCTTCGTTTCGGTTTCGCGGCGGCGGCGGCGTTGTTGGCTGAGGGGACCCGGG
ACACCTGAATGCCCCCGGCCCCGGCTCCTCCGACGCGATGGGGAAGGTGCTATCCAAAAT
CTTCGGGAACAAGGAAATGCGGATCCTCATGTTGGGCCTGGACGCGGCCGGCAAGACAAC
AATCCTGTACAAGTTGAAGCTGGGCCAGTCGGTGACCACCATTCCCACTGTGGGTTTCAA
CGTGGAGACGGTGACTTACAAAAATGTCAAGTTCAACGTATGGGATGTGGGCGGCCAGGA
CAAGATCCGGCCGCTCTGGCGGCATTACTACACTGGGACCCAAGGTCTCATCTTCGTAGT
GGACTGCGCCGACCGCGACCGCATCGATGAGGCTCGCCAGGAGCTGCACCGCATTATCAA
TGACCGGGAGATGAGGGACGCCATAATCCTCATCTTCGCCAACAAGCAGGACCTGCCCGA
TGCCATGAAACCCCACGAGATCCAGGAGAAACTGGGCCTGACCCGGATTCGGGACAGGAA
CTGGTATGTGCAGCCCTCCTGTGCCACCTCAGGGGACGGACTCTATGAGGGGCTCACATG
GTTAACCTCTAACTACAAATCTTAATGAGCATTCTCCACCCATCCCCTGGAAGGAGAGAA
ATCAAAAACCCATTCATAGGATTATCGCCACCATCACCTCTTTCAATTGCCACTTTCTCT
TCTTTTGAATTTGAACTCTGGAGTTACTGTTCTACAGTTTGGCGGGGACGGGGCTTGGGG
GTTTTCTCTTTTGTTTGTTTCCCTTTCTTTTTCCTTTTTTTTTTTTTTTTTTTGTTGGCT
TTGCGTTAGGATGGCTCTGATCTGACATTTGACATGAACACAAAGTTGCCAAGATGCTCC
TTGTTGACTTCCAGCAGAATGGGAATGGGGGAAACACAGCAGTTCTTGGGTAAAAGTCCC
TTTGTAATAATAGGTTTGGGATTTTTTTATTTCGAGAGAATCTTTCATTTTCCTATGTAT
GCTTTTTTCCTTTTTTGCCCAGTTTCCTTATCACTTGCTGTAGATGGCTTATTTTGCATT
CATGCAGACTATGTTGCAAGTCTGTTTCATCTAGTAAACTGAAAATTATTGCTTAATCAA
ACTGCCGTTTGTCTTTTATATTTAAGGCCTTCCCCCCCCTTCCTTATGAGTTCTAACTTA
GTAATTTCAAATGTGACCTTTTATATCTAAGACCAGTATAGTAAACTTAGCCCACAGTGG
CAAATAATGAGTAATATTGTAATATGTTCCAGTTGCACCTCAGTATGTTAAACAGGTAAT
GTAAGAAGTTCTCTGAAATGTCAGCAAGTAAGTTCTGAAACACATCATGCATGAGTAGGA
ATAAAC
>gi|4502211|gb|NP_001654.1|ARF6 175 aa linear ADP-ribosyla-
tion factor 6 [Homo sapiens].
MGKVLSKIFGNKEMRILMLGLDAAGKTTILYKLKLGQSVTTIPTVGFNVETVTYKNVKFN
VWDVGGQDKIRPLWRHYYTGTQGLIFVVDCADRDRIDEARQELHRIINDREMRDAIILIF
ANKQDLPDAMKPHEIQEKLGLTRIRDRNWYVQPSCATSGDGLYEGLTWLTSNYKS
>gi|23510442|gb|NM_003809.2|TNFSF12 1407 bp mRNA Homo
sapiens tumor necrosis factor (ligand) superfamily, member
12 (TNFSF12), transcript variant 1, mRNA.
CTCTCCCCGGCCCGATCCGCCCGCCGGCTCCCCCTCCCCCGATCCCTCGGGTCCCGGGAT
GGGGGGGCGGTGAGGCAGGCACAGCCCCCCGCCCCCATGGCCGCCCGTCGGAGCCAGAGG
CGGAGGGGGCGCCGGGGGGAGCCGGGCACCGCCCTGCTGGTCCCGCTCGCGCTGGGCCTG
GGCCTGGCGCTGGCCTGCCTCGGCCTCCTGCTGGCCGTGGTCAGTTTGGGGAGCCGGGCA
TCGCTGTCCGCCCAGGAGCCTGCCCAGGAGGAGCTGGTGGCAGAGGAGGACCAGGACCCG
TCGGAACTGAATCCCCAGACAGAAGAAAGCCAGGATCCTGCGCCTTTCCTGAACCGACTA
GTTCGGCCTCGCAGAAGTGCACCTAAAGGCCGGAAAACACGGGCTCGAAGAGCGATCGCA
GCCCATTATGAAGTTCATCCACGACCTGGACAGGACGGAGCGCAGGCAGGTGTGGACGGG
ACAGTGAGTGGCTGGGAGGAAGCCAGAATCAACAGCTCCAGCCCTCTGCGCTACAACCGC
CAGATCGGGGAGTTTATAGTCACCCGGGCTGGGCTCTACTACCTGTACTGTCAGGTGCAC
TTTGATGAGGGGAAGGCTGTCTACCTGAAGCTGGACTTGCTGGTGGATGGTGTGCTGGCC
CTGCGCTGCCTGGAGGAATTCTCAGCCACTGCGGCGAGTTCCCTCGGGCCCCAGCTCCGC
CTCTGCCAGGTGTCTGGGCTGTTGGCCCTGCGGCCAGGGTCCTCCCTGCGGATCCGCACC
CTCCCCTGGGCCCATCTCAAGGCTGCCCCCTTCCTCACCTACTTCGGACTCTTCCAGGTT
CACTGAGGGGCCCTGGTCTCCCCGCAGTCGTCCCAGGCTGCCGGCTCCCCTCGACAGCTC
TCTGGGCACCCGGTCCCCTCTGCCCCACCCTCAGCCGCTCTTTGCTCCAGACCTGCCCCT
CCCTCTAGAGGCTGCCTGGGCCTGTTCACGTGTTTTCCATCCCACATAAATACAGTATTC
CCACTCTTATCTTACAACTCCCCCACCGCCCACTCTCCACCTCACTAGCTCCCCAATCCC
TGACCCTTTGAGGCCCCCAGTGATCTCGACTCCCCCCTGGCCACAGACCCCCAGGGCATT
GTGTTCACTGTACTCTGTGGGCAAGGATGGGTCCAGAAGACCCCACTTCAGGCACTAAGA
GGGGCTGGACCTGGCGGCAGGAAGCCAAAGAGACTGGGCCTAGGCCAGGAGTTCCCAAAT
GTGAGGGGCGAGAAACAAGACAAGCTCCTCCCTTGAGAATTCCCTGTGGATTTTTAAAAC
AGATATTATTTTTATTATTATTGTGACAAAATGTTGATAAATGGATATTAAATAGAATAA
GTCATAAAAAAAAAAAAAAAAAAAAAA
>gi|4507597|gb|NP_003800.1|TNFSF12 249 aa linear tumor
necrosis factor (ligand) superfamily, member 12 isoform 1
precursor; APO3/DR3 ligand; TNF-related WEAK inducer of
apoptosis [Homo sapiens].
MAARRSQRRRGRRGEPGTALLVPLALGLGLALACLGLLLAVVSLGSRASLSAQEPAQEEL
VAEEDQDPSELNPQTEESQDPAPFLNRLVRPRRSAPKGRKTRARRAIAAHYEVHPRPGQD
GAQAGVDGTVSGWEEARINSSSPLRYNRQIGEFIVTRAGLYYLYCQVHFDEGKAVYLKLD
LLVDGVLALRCLEEFSATAASSLGPQLRLCQVSGLLALRPGSSLRIRTLPWAHLKAAPFL
TYFGLFQVH
>gi|11496238|gb|NM_021975.1|RELA. 2444 bp mRNA Homo sapiens
v-rel reticuloendotheliosis viral oncogene homolog A,
nuclear factor of kappa light polypeptide gene enhancer in
B-cells 3, p65 (avian) (RELA), mRNA.
GGCACGAGGCGGGGCCGGGTCGCAGCTGGGCCCGCGGCATGGACGAACTGTTCCCCCTCA
TCTTCCCGGCAGAGCAGCCCAAGCAGCGGGGCATGCGCTTCCGCTACAAGTGCGAGGGGC
GCTCCGCGGGCAGCATCCCAGGCGAGAGGAGCACAGATACCACCAAGACCCACCCCACCA
TCAAGATCAATGGCTACACAGGACCAGGGACAGTGCGCATCTCCCTGGTCACCAAGGACC
CTCCTCACCGGCCTCACCCCCACGAGCTTGTAGGAAAGGACTGCCGGGATGGCTTCTATG
AGGCTGAGCTCTGCCCGGACCGCTGCATCCACAGTTTCCAGAACCTGGGAATCCAGTGTG
TGAAGAAGCGGGACCTGGAGCAGGCTATCAGTCAGCGCATCCAGACCAACAACAACCCCT
TCCAAGTTCCTATAGAAGAGCAGCGTGGGGACTACGACCTGAATGCTGTGCGGCTCTGCT
TCCAGGTGACAGTGCGGGACCCATCAGGCAGGCCCCTCCGCCTGCCGCCTGTCCTTTCTC
ATCCCATCTTTGACAATCGTGCCCCCAACACTGCCGAGCTCAAGATCTGCCGAGTGAACC
GAAACTCTGGCAGCTGCCTCGGTGGGGATGAGATCTTCCTACTGTGTGACAAGGTGCAGA
AAGAGGACATTGAGGTGTATTTCACGGGACCAGGCTGGGAGGCCCGAGGCTCCTTTTCGC
AAGCTGATGTGCACCGACAAGTGGCCATTGTGTTCCGGACCCCTCCCTACGCAGACCCCA
GCCTGCAGGCTCCTGTGCGTGTCTCCATGCAGCTGCGGCGGCCTTCCGACCGGGAGCTCA
GTGAGCCCATGGAATTCCAGTACCTGCCAGATACAGACGATCGTCACCGGATTGAGGAGA
AACGTAAAAGGACATATGAGACCTTCAAGAGCATCATGAAGAAGAGTCCTTTCAGCGGAC
CCACCGACCCCCGGCCTCCACCTCGACGCATTGCTGTGCCTTCCCGCAGCTCAGCTTCTG
TCCCCAAGCCAGCACCCCAGCCCTATCCCTTTACGTCATCCCTGAGCACCATCAACTATG
ATGAGTTTCCCACCATGGTGTTTCCTTCTGGGCAGATCAGCCAGGCCTCGGCCTTGGCCC
CGGCCCCTCCCCAAGTCCTGCCCCAGGCTCCAGCCCCTGCCCCTGCTCCAGCCATGGTAT
CAGCTCTGGCCCAGGCCCCAGCCCCTGTCCCAGTCCTAGCCCCAGGCCCTCCTCAGGCTG
TGGCCCCACCTGCCCCCAAGCCCACCCAGGCTGGGGAAGGAACGCTGTCAGAGGCCCTGC
TGCAGCTGCAGTTTGATGATGAAGACCTGGGGGCCTTGCTTGGCAACAGCACAGACCCAG
CTGTGTTCACAGACCTGGCATCCGTCGACAACTCCGAGTTTCAGCAGCTGCTGAACCAGG
GCATACCTGTGGCCCCCCACACAACTGAGCCCATGCTGATGGAGTACCCTGAGGCTATAA
CTCGCCTAGTGACAGCCCAGAGGCCCCCCGACCCAGCTCCTGCTCCACTGGGGGCCCCGG
GGCTCCCCAATGGCCTCCTTTCAGGAGATGAAGACTTCTCCTCCATTGCGGACATGGACT
TCTCAGCCCTGCTGAGTCAGATCAGCTCCTAAGGGGGTGACGCCTGCCCTCCCCAGAGCA
CTGGTTGCAGGGGATTGAAGCCCTCCAAAAGCACTTACGGATTCTGGTGGGGTGTGTTCC
AACTGCCCCCAACTTTGTGGATGTCTTCCTTGGAGGGGGGAGCCATATTTTATTCTTTTA
TTGTCAGTATCTGTATCTCTCTCTCTTTTTGGAGGTGCTTAAGCAGAAGCATTAACTTCT
CTGGAAAGGGGGGAGCTGGGGAAACTCAAACTTTTCCCCTGTCCTGATGGTCAGCTCCCT
TCTCTGTAGGGAACTGTGGGGTCCCCCATCCCCATCCTCCAGCTTCTGGTACTCTCCTAG
AGACAGAAGCAGGCTGGAGGTAAGGCCTTTGAGCCCACAAAGCCTTATCAAGTGTCTTCC
ATCATGGATTCATTACAGCTTAATCAAAATAACGCCCCAGATACCAGCCCCTGTATGGCA
CTGGCATTGTCCCTGTGCCTAACACCAGCGTTTGAGGGGCTGCCTTCCTGCCCTACAGAG
GTCTCTGCCGGCTCTTTCCTTGCTCAACCATGGCTGAAGGAAACAGTGCAACAGCACTGG
CTCTCTCCAGGATCCAGAAGGGGTTTGGTCTGGACTTCCTTGCTCTCCCCTCTTCTCAAG
TGCCTTAATAGTAGGGTAAGTTGTTAAGAGTGGGGGAGAGCAGGCTGGCAGCTCTCCAGT
CAGGAGGCATAGTTTTTAGTGAACAATCAAAGCACTTGGACTCTTGCTCTTTCTACTCTG
AACTAATAAAGCTGTTGCCAAGCTGGACGGCACGAGCTCGTGCC
>gi|11496239|gb|NP_068810.1|RELA 537 aa linear v-rel
reticuloendotheliosis viral oncogene homolog A, nuclear
factor of kappa light polypeptide gene enhancer in B-cells
3, p65; v-rel avian reticuloendotheliosis viral oncogene
homolog A (nuclear factor of kappa light polypeptide gene
enhancer in B-cells 3 (p65)) [Homo sapiens].
MDELFPLIFPAEQPKQRGMRFRYKCEGRSAGSIPGERSTDTTKTHPTIKINGYTGPGTVR
ISLVTKDPPHRPHPHELVGKDCRDGFYEAELCPDRCIHSFQNLGIQCVKKRDLEQAISQR
IQTNNNPFQVPIEEQRGDYDLNAVRLCFQVTVRDPSGRPLRLPPVLSHPIFDNRAPNTAE
LKICRVNRNSGSCLGGDEIFLLCDKVQKEDIEVYFTGPGWEARGSFSQADVHRQVAIVFR
TPPYADPSLQAPVRVSMQLRRPSDRELSEPMEFQYLPDTDDRHRIEEKRKRTYETFKSIM
KKSPFSGPTDPRPPPRRIAVPSRSSASVPKPAPQPYPFTSSLSTINYDEFPTMVFPSGQI
SQASALAPAPPQVLPQAPAPAPAPAMVSALAQAPAPVPVLAPGPPQAVAPPAPKPTQAGE
GTLSEALLQLQFDDEDLGALLGNSTDPAVFTDLASVDNSEFQQLLNQGIPVAPHTTEPML
MEYPEAITRLVTAQRPPDPAPAPLGAPGLPNGLLSGDEDFSSIADMDFSALLSQISS
>gi|23312372|gb|NM_001065.2|TNFRSF1A 2236 bp mRNA Homo
sapiens tumor necrosis factor receptor superfamily, member
1A (TNFRSF1A), mRNA.
GCTGTTGCAACACTGCCTCACTCTTCCCCTCCCACCTTCTCTCCCCTCCTCTCTGCTTTA
ATTTTCTCAGAATTCTCTGGACTGAGGCTCCAGTTCTGGCCTTTGGGGTTCAAGATCACT
GGGACCAGGCCGTGATCTCTATGCCCGAGTCTCAACCCTCAACTGTCACCCCAAGGCACT
TGGGACGTCCTGGACAGACCGAGTCCCGGGAAGCCCCAGCACTGCCGCTGCCACACTGCC
CTGAGCCCAAATGGGGGAGTGAGAGGCCATAGCTGTCTGGCATGGGCCTCTCCACCGTGC
CTGACCTGCTGCTGCCACTGGTGCTCCTGGAGCTGTTGGTGGGAATATACCCCTCAGGGG
TTATTGGACTGGTCCCTCACCTAGGGGACAGGGAGAAGAGAGATAGTGTGTGTCCCCAAG
GAAAATATATCCACCCTCAAAATAATTCGATTTGCTGTACCAAGTGCCACAAAGGAACCT
ACTTGTACAATGACTGTCCAGGCCCGGGGCAGGATACGGACTGCAGGGAGTGTGAGAGCG
GCTCCTTCACCGCTTCAGAAAACCACCTCAGACACTGCCTCAGCTGCTCCAAATGCCGAA
AGGAAATGGGTCAGGTGGAGATCTCTTCTTGCACAGTGGACCGGGACACCGTGTGTGGCT
GCAGGAAGAACCAGTACCGGCATTATTGGAGTGAAAACCTTTTCCAGTGCTTCAATTGCA
GCCTCTGCCTCAATGGGACCGTGCACCTCTCCTGCCAGGAGAAACAGAACACCGTGTGCA
CCTGCCATGCAGGTTTCTTTCTAAGAGAAAACGAGTGTGTCTCCTGTAGTAACTGTAAGA
AAAGCCTGGAGTGCACGAAGTTGTGCCTACCCCAGATTGAGAATGTTAAGGGCACTGAGG
ACTCAGGCACCACAGTGCTGTTGCCCCTGGTCATTTTCTTTGGTCTTTGCCTTTTATCCC
TCCTCTTCATTGGTTTAATGTATCGCTACCAACGGTGGAAGTCCAAGCTCTACTCCATTG
TTTGTGGGAAATCGACACCTGAAAAAGAGGGGGAGCTTGAAGGAACTACTACTAAGCCCC
TGGCCCCAAACCCAAGCTTCAGTCCCACTCCAGGCTTCACCCCCACCCTGGGCTTCAGTC
CCGTGCCCAGTTCCACCTTCACCTCCAGCTCCACCTATACCCCCGGTGACTGTCCCAACT
TTGCGGCTCCCCGCAGAGAGGTGGCACCACCCTATCAGGGGGCTGACCCCATCCTTGCGA
CAGCCCTCGCCTCCGACCCCATCCCCAACCCCCTTCAGAAGTGGGAGGACAGCGCCCACA
AGCCACAGAGCCTAGACACTGATGACCCCGCGACGCTGTACGCCGTGGTGGAGAACGTGC
CCCCGTTGCGCTGGAAGGAATTCGTGCGGCGCCTAGGGCTGAGCGACCACGAGATCGATC
GGCTGGAGCTGCAGAACGGGCGCTGCCTGCGCGAGGCGCAATACAGCATGCTGGCGACCT
GGAGGCGGCGCACGCCGCGGCGCGAGGCCACGCTGGAGCTGCTGGGACGCGTGCTCCGCG
ACATGGACCTGCTGGGCTGCCTGGAGGACATCGAGGAGGCGCTTTGCGGCCCCGCCGCCC
TCCCGCCCGCGCCCAGTCTTCTCAGATGAGGCTGCGCCCCTGCGGGCAGCTCTAAGGACC
GTCCTGCGAGATCGCCTTCCAACCCCACTTTTTTCTGGAAAGGAGGGGTCCTGCAGGGGC
AAGCAGGAGCTAGCAGCCGCCTACTTGGTGCTAACCCCTCGATGTACATAGCTTTTCTCA
GCTGCCTGCGCGCCGCCGACAGTCAGCGCTGTGCGCGCGGAGAGAGGTGCGCCGTGGGCT
CAAGAGCCTGAGTGGGTGGTTTGCGAGGATGAGGGACGCTATGCCTCATGCCCGTTTTGG
GTGTCCTCACCAGCAAGGCTGCTCGGGGGCCCCTGGTTCGTCCCTGAGCCTTTTTCACAG
TGCATAAGCAGTTTTTTTTGTTTTTGTTTTGTTTTGTTTTGTTTTTAAATCAATCATGTT
ACACTAATAGAAACTTGGCACTCCTGTGCCCTCTGCCTGGACAAGCACATAGCAAGCTGA
ACTGTCCTAAGGCAGGGGCGAGCACGGAACAATGGGGCCTTCAGCTGGAGCTGTGGACTT
TTGTACATACACTAAAATTCTGAAGTTAAAGCTCTGCTCTTGGAAAAAAAAAAAAAAAAA
AAAAAAAAAAAAAAAA
>gi|4507575|gb|NP_001056.1|TNFRSF1A 455 aa linear tumor
necrosis factor receptor 1 precursor; tumor necrosis factor
receptor type 1; tumor necrosis factor-alpha receptor; tumor
necrosis factor binding protein 1 [Homo sapiens].
MGLSTVPDLLLPLVLLELLVGIYPSGVIGLVPHLGDREKRDSVCPQGKYIHPQNNSICCT
KCHKGTYLYNDCPGPGQDTDCRECESGSFTASENHLRHCLSCSKCRKEMGQVEISSCTVD
RDTVCGCRKNQYRHYWSENLFQCFNCSLCLNGTVHLSCQEKQNTVCTCHAGFFLRENECV
SCSNCKKSLECTKLCLPQIENVKGTEDSGTTVLLPLVIFFGLCLLSLLFIGLMYRYQRWK
SKLYSIVCGKSTPEKEGELEGTTTKPLAPNPSFSPTPGFTPTLGFSPVPSSTFTSSSTYT
PGDCPNFAAPRREVAPPYQGADPILATALASDPIPNPLQKWEDSAHKPQSLDTDDPATLY
AVVENVPPLRWKEFVRRLGLSDHEIDRLELQNGRCLREAQYSMLATWRRRTPRREATLEL
LGRVLRDMDLLGCLEDIEEALCGPAALPPAPSLLR
>gi|4506738|gb|NM_003952.1|RPS6KB2 1735 bp mRNA Homo sapiens
ribosomal protein S6 kinase, 70kDa, polypeptide 2 (RPS6KB2),
mRNA.
AGAGACTCGTGCCGAATGGCACGAGGCCGACGGGCCCGCGGGGCCGGCGCCGCCATGGCG
GCCGTGTTTGATTTGGATTTGGAGACGGAGGAAGGCAGCGAGGGCGAGGGCGAGCCAGAG
CTCAGCCCCGCGGACGCATGTCCCCTTGCCGAGTTGAGGGCAGCTGGCCTAGAGCCTGTG
GGACACTATGAAGAGGTGGAGCTGACTGAGACCAGCGTGAACGTTGGCCCAGAGCGCATC
GGGCCCCACTGCTTTGAGCTGCTGCGTGTGCTGGGCAAGGGGGGCTATGGCAAGGTGTTC
CAGGTGCGAAAGGTGCAAGGCACCAACTTGGGCAAAATATATGCCATGAAAGTCCTAAGG
AAGGCCAAAATTGTGCGCAATGCCAAGGACACAGCACACACACGGGCTGAGCGGAACATT
CTAGAGTCAGTGAAGCACCCCTTTATTGTGGAACTGGCCTATGCCTTCCAGACTGGTGGC
AAACTCTACCTCATCCTTGAGTGCCTCAGTGGTGGCGAGCTCTTCACGCATCTGGAGCGA
GAGGGCATCTTCCTGGAAGATACGGCCTGCTTCTACCTGGCTGAGATCACGCTGGCCCTG
GGCCATCTCCACTCCCAGGGCATCATCTACCGGGACCTCAAGCCCGAGAACATCATGCTC
AGCAGCCAGGGCCACATCAAACTGACCGACTTTGGACTCTGCAAGGAGTCTATCCATGAG
GGCGCCGTCACTCACACCTTCTGCGGCACCATTGAGTACATGGCCCCTGAGATTCTGGTG
CGCAGTGGCCACAACCGGGCTGTGGACTGGTGGAGCCTGGGGGCCCTGATGTACGACATG
CTCACTGGATCGCCGCCCTTTACCGCAGAGAACCGGAAGAAAACCATGGATAAGATCATC
AGGGGCAAGCTGGCACTGCCCCCCTACCTCACCCCAGATGCCCGGGACCTTGTCAAAAAG
TTTCTGAAACGGAATCCCAGCCAGCGGATTGGGGGTGGCCCAGGGGATGCTGCTGATGTG
CAGAGACATCCCTTTTTCCGGCACATGAATTGGGACGACCTTCTGGCCTGGCGTGTGGAC
CCCCCTTTCAGGCCCTGTCTGCAGTCAGAGGAGGACGTGAGCCAGTTTGATACCCGCTTC
ACACGGCAGACGCCGGTGGACAGTCCTGATGACACAGCCCTCAGCGAGAGTGCCAACCAG
GCCTTCCTGGGCTTCACATACGTGGCGCCGTCTGTCCTGGACAGCATCAAGGAGGGCTTC
TCCTTCCAGCCCAAGCTGCGCTCACCCAGGCGCCTCAACAGTAGCCCCCGGGTCCCCGTC
AGCCCCCTCAAGTTCTCCCCTTTTGAGGGGTTTCGGCCCAGCCCCAGCCTGCCGGAGCCC
ACGGAGCTACCTCTACCTCCACTCCTGCCACCGCCGCCGCCCTCGACCACCGCCCCTCTC
CCCATCCGTCCCCCCTCAGGGACCAAGAAGTCCAAGAGGGGCCGTGGGCGTCCAGGGCGC
TAGGAAGCCGGGTGGGGGTGAGGGTAGCCCTTGAGCCCTGTCCCTGCGGCTGTGAGAGCA
GCAGGACCCTGGGCCAGTTCCAGAGACCTGGGGGTGTGTCTGGGGGTGGGGTGTGAGTGC
GTATGAAAGTGTGTGTCTGCTGGGGCAGCTGTGCCCCTGAATCATGGGCACGGAGGGCCG
CCCGCCACACCCCGCGCTCAACTGCTCCCGTGGAAGATTAAAGGGCTGAATCATG
>gi|4506739|gb|NP_003943.1|RPS6KB2 495 aa linear ribosomal
protein S6 kinase, 70kDa, polypeptide 2; ribosomal protein
S6 kinase, 70kD, polypeptide 2; p70 ribosomal S6 kinase beta
[Homo sapiens].
MARGRRARGAGAAMAAVFDLDLETEEGSEGEGEPELSPADACPLAELRAAGLEPVGHYEE
VELTETSVNVGPERIGPHCFELLRVLGKGGYGKVFQVRKVQGTNLGKIYAMKVLRKAKIV
RNAKDTAHTRAERNILESVKHPFIVELAYAFQTGGKLYLILECLSGGELFTHLEREGIFL
EDTACFYLAEITLALGHLHSQGIIYRDLKPENIMLSSQGHIKLTDFGLCKESIHEGAVTH
TFCGTIEYMAPEILVRSGHNRAVDWWSLGALMYDMLTGSPPFTAENRKKTMDKIIRGKLA
LPPYLTPDARDLVKKFLKRNPSQRIGGGPGDAADVQRHPFFRHMNWDDLLAWRVDPPFRP
CLQSEEDVSQFDTRFTRQTPVDSPDDTALSESANQAFLGFTYVAPSVLDSIKEGFSFQPK
LRSPRRLNSSPRVPVSPLKFSPFEGFRPSPSLPEPTELPLPPLLPPPPPSTTAPLPIRPP
SGTKKSKRGRGRPGR
>gi|11995473|gb|NM_019884.1|GSK3A 2169 bp mRNA Homo sapiens
glycogen synthase kinase 3 alpha (GSK3A), mRNA.
GCCAGAGCGGCGCGGCCTGGAAGAGGCCAGGGCCCGGGGGAGGCGACGGCAGCGGCGGCG
GCTGGGGCAGCCCGGGCAGCCCGAGCCCCGCAGCCTGGGCCTGTGCTCGGCGCCATGAGC
GGCGGCGGGCCTTCGGGAGGCGGCCCTGGGGGCTCGGGCAGGGCGCGGACTAGCTCGTTC
GCGGAGCCCGGCGGCGGAGGCGGAGGAGGCGGCGGCGGCCCCGGAGGCTCGGCCTCCGGC
CCAGGCGGCACCGGCGGCGGAAAGGCATCTGTCGGGGCCATGGGTGGGGGCGTCGGGGCC
TCGAGCTCCGGGGGTGGACCCGGCGGCAGCGGCGGAGGAGGCAGCGGAGGCCCCGGCGCA
GGCACTAGCTTCCCGCCGCCCGGGGTGAAGCTGGGCCGTGACAGCGGGAAGGTGACCACA
GTCGTAGCCACTCTAGGCCAAGGCCCAGAGCGCTCCCAAGAAGTGGCTTACACGGACATC
AAAGTGATTGGCAATGGCTCATTTGGGGTCGTGTACCAGGCACGGCTGGCAGAGACCAGG
GAACTAGTCGCCATCAAGAAGGTTCTCCAGGACAAGAGGTTCAAGAACCGAGAGCTGCAG
ATCATGCGTAAGCTGGACCACTGCAATATTGTGAGGCTGAGATACTTTTTCTACTCCAGT
GGCGAGAAGAAAGACGAGCTTTACCTAAATCTGGTGCTGGAATATGTGCCCGAGACAGTG
TACCGGGTGGCCCGCCACTTCACCAAGGCCAAGTTGACCATCCCTATCCTCTATGTCAAG
GTGTACATGTACCAGCTCTTCCGCAGCTTGGCCTACATCCACTCCCAGGGCGTGTGTCAC
CGCGACATCAAGCCCCAGAACCTGCTGGTGGACCCTGACACTGCTGTCCTCAAGCTCTGC
GATTTTGGCAGTGCAAAGCAGTTGGTCCGAGGGGAGCCCAATGTCTCCTACATCTGTTCT
CGCTACTACCGGGCCCCAGAGCTCATCTTTGGAGCCACTGATTACACCTCATCCATCGAT
GTTTGGTCAGCTGGCTGTGTACTGGCAGAGCTCCTCTTGGGCCAGCCCATCTTCCCTGGG
GACAGTGGGGTGGACCAGCTGGTGGAGATCATCAAGGTGCTGGGAACACCAACCCGGGAA
CAAATCCGAGAGATGAACCCCAACTACACGGAGTTCAAGTTCCCTCAGATTAAAGCTCAC
CCCTGGACAAAGGTGTTCAAATCTCGAACGCCGCCAGAGGCCATCGCGCTCTGCTCTAGC
CTGCTGGAGTACACCCCATCCTCAAGGCTCTCCCCACTAGAGGCCTGTGCGCACAGCTTC
TTTGATGAACTGCGATGTCTGGGAACCCAGCTGCCTAACAACCGCCCACTTCCCCCTCTC
TTCAACTTCAGTGCTGGTGAACTCTCCATCCAACCGTCTCTCAACGCCATTCTCATCCCT
CCTCACTTGAGGTCCCCCAGCGGCACTACCACCCTCACCCCGTCCTCACAAGCTTTAACT
GAGACTCCGACCAGCTCAGACTGGCAGTCGACCGATGCCACACCTACCCTCACTAACTCC
TCCTGAGGGCCCCACCAAGCACCCTTCCACTTCCATCTGGGAGCCCCAAGAGGGCGTGGG
AAGGGGGGCCATAGCCCATCAAGCTCCTGCCCTGGCTGGGCCCCTAGACTAGAGGGCAGA
GGTAAATGAGTCCCTGTCCCCACCTCCAGTCCCTCCCTCACCAGCCTCACCCCTGTGGTG
GGCTTTTTAAGAGGATTTTAACTGGTTGTGGGGAGGGAAGAGAAGGACAGGGTGTTGGGG
GGATGAGGACCTCCTACCCCCTTGGCCCCCTCCCCTCCCCCAGACCTCCACCTCCTCCAG
ACCCCCTCCCCTCCTGTGTCCCTTGTAAATAGAACCAGCCCAGCCCGTCTCCTCTTCCCT
TCCCTGGCCCCCGGGTGTAAATAGATTGTTATAATTTTTTTCTTAAAGAAAACGTCGATT
CGCACCGTCCAACCTGCCCCGCCCCTCCTACAGCTGTAACTCCCCTCCTGTCCTCTGCCC
CCAAGGTCTACTCCCTCCTCACCCCACCCTGGAGGGCCAGGGGAGTGGAGAGAGCTCCTG
ATGTCTTAGTTTCCACAGTAAGGTTTGCCTGTGTACAGACCTCCGTTCAATAAATTATTG
GCATGAAAA
>gi|11995474|gb|NP_063937.1|GSK3A 483 aa linear glycogen
synthase kinase 3 alpha [Homo sapiens].
MSGGGPSGGGPGGSGRARTSSFAEPGGGGGGGGGGPGGSASGPGGTGGGKASVGAMGGGV
GASSSGGGPGGSGGGGSGGPGAGTSFPPPGVKLGRDSGKVTTVVATLGQGPERSQEVAYT
DIKVIGNGSFGVVYQARLAETRELVAIKKVLQDKRFKNRELQIMRKLDHCNIVRLRYFFY
SSGEKKDELYLNLVLEYVPETVYRVARHFTKAKLTIPILYVKVYMYQLFRSLAYIHSQGV
CHRDIKPQNLLVDPDTAVLKLCDFGSAKQLVRGEPNVSYICSRYYRAPELIFGATDYTSS
IDVWSAGCVLAELLLGQPIFPGDSGVDQLVEIIKVLGTPTREQIREMNPNYTEFKFPQIK
AHPWTKVFKSRTPPEAIALCSSLLEYTPSSRLSPLEACAHSFFDELRCLGTQLPNNRPLP
PLFNFSAGELSIQPSLNAILIPPHLRSPSGTTTLTPSSQALTETPTSSDWQSTDATPTLT
NSS
>gi|7019350|gb|NM_013246.1|CLC 1689 bp mRNA Homo sapiens
cardiotrophin-like cytokine (CLC), mRNA.
GCCTCCGGGAGAGGAGCCGCACCCGGCCGGCCCGGCCCCAGCCCCATGGACCTCCGAGCA
GGGGACTCGTGGGGGATGTTAGCGTGCCTGTGCACGGTGCTCTGGCACCTCCCTGCAGTG
CCAGCTCTCAATCGCACAGGGGACCCAGGGCCTGGCCCCTCCATCCAGAAAACCTATGAC
CTCACCCGCTACCTGGAGCACCAACTCCGCAGCTTGGCTGGGACCTATCTGAACTACCTG
GGCCCCCCTTTCAACGAGCCAGACTTCAACCCTCCCCGCCTGGGGGCAGAGACTCTGCCC
AGGGCCACTGTTGACTTGGAGGTGTGGCGAAGCCTCAATGACAAACTGCGGCTGACCCAG
AACTACGAGGCCTACAGCCACCTTCTGTGTTACTTGCGTGGCCTCAACCGTCAGGCTGCC
ACTGCTGAGCTGCGCCGCAGCCTGGCCCACTTCTGCACCAGCCTCCAGGGCCTGCTGGGC
AGCATTGCGGGCGTCATGGCAGCTCTGGGCTACCCACTGCCCCAGCCGCTGCCTGGGACT
GAACCCACTTGGACTCCTGGCCCTGCCCACAGTGACTTCCTCCAGAAGATGGACGACTTC
TGGCTGCTGAAGGAGCTGCAGACCTGGCTGTGGCGCTCGGCCAAGGACTTCAACCGGCTC
AAGAAGAAGATGCAGCCTCCAGCAGCTGCAGTCACCCTGCACCTGGGGGCTCATGGCTTC
TGACTTCTGACCTTCTCCTCTTCGCTCCCCCTTCAAACCCTGCTCCCACTTTGTGAGAGC
CAGCCCTGTATGCCAACACCTGTTGAGCCAGGAGACAGAAGCTGTGAGCCTCTGGCCCTT
TCCTGGACCGGCTGGGCGTGTGATGCGATCAGCCCTGTCTCCTCCCCACCTCCCAAAGGT
CTACCGAGCTGGGGAGGAGGTACAGTAGGCCCTGTCCTGTCCTGTTTCTACAGGAAGTCA
TGCTCGAGGGAGTGTGAAGTGGTTCAGGTTGGTGCAGAGGCGCTCATGGCCTCCTGCTTC
TTGCCTACCACTTGGCCAGTGCCCACCCAGCCCCTCAGGTGGCACATCTGGAGGGCAGGG
GTTGAGGGGCCACCACCACACATGCCTTTCTGGGGTGAAGCCCTTTGGCTGCCCCACTCT
CCTTGGATGGGTGTTGCTCCCTTATCCCCAAATCACTCTATACATCCAATTCAGGAAACA
AACATGGTGGCAATTCTACACAAAAAGAGATGAGATTAACAGTGCAGGGTTGGGGTCTGC
ATTGGAGGTGCCCTATAAACCAGAAGAGAAAATACTGAAAGCACAGGGGCAGGGACAGAC
CAGACCAGACCCAGGAGTCTCCAAAGCACAGAGTGGCAAACAAAACCCGAGCTGAGCATC
AGGACCTTGCCTCGAATTGTCTTCCAGTATTACGGTGCCTCTTCTCTGCCCCCTTTCCCA
GGGTATCTGTGGGTTGCCAGGCTGGGGAGGGCAACCATAGCCACACCACAGGATTTCCTG
AAAGTTTACAATGCAGTAGCATTTTGGGGTGTAGGGTGGCAGCTCCCCAAGGCCCTGCCC
CCCAGCCCCACCCACTCATGACTCTAAGTGTGTTGTATTAATATTTATTTATTTGGAGAT
GTTATTTATTAGATGATATTTATTGCAGAATTTCTATTCTTGTATTAACAAATAAAATGC
TTGCCCCAG
>gi|7019351|gb|NP_037378.1|CLC 225 aa linear cardiotrophin-
like cytokine; neurotrophin-1/B-cell stimulating factor-3
[Homo sapiens].
MDLRAGDSWGMLACLCTVLWHLPAVPALNRTGDPGPGPSIQKTYDLTRYLEHQLRSLAGT
YLNYLGPPFNEPDFNPPRLGAETLPRATVDLEVWRSLNDKLRLTQNYEAYSHLLCYLRGL
NRQAATAELRRSLAHFCTSLQGLLGSIAGVMAALGYPLPQPLPGTEPTWTPGPAHSDFLQ
KMDDFWLLKELQTWLWRSAKDFNRLKKKMQPPAAAVTLHLGAHGF
>gi|22068574|gb|XM_036493.3|ZNF213 3073 bp mRNA Homo sapiens
zinc finger protein 213 (ZNF213), mRNA.
GGCCTCTGGCCGCCTGGCTCCAACATCAAGCACCGGGCTCCGAGTGGCCGGGATCAGCGC
CCCGAGGCAGAGGCCGGAGGGCGCGCGCACTGCTAGGAAGTGCTGGTCCCCCGCGCCGCT
CTGCCAGCTTGGTCCCCCGGCAGACGCCCCTGTACGATCGCCGCTCGCCCCGCGGGCGAG
GCTGCGGTGGACAGCGCGGGGCTCCGGCTGGCTCGCCTTCCCGCCTGCCGTGTCCTGCTG
AGCGACCCTGGAGTACACATCCAGATGCCAGCCCAGCTACCACAGGGGATCCCTCTGGGA
GACTGAAAGTACAGGTTCTGGGGCCCAGGTTGAAGCCGACCAACCCTGAGCCTCAGGCCA
GGGGAATGGCAGCCCCCTTGGAGGCCCAGGACCAGGCCCCTGGGGAGGGAGAAGGGCTTC
TGATTGTGAAAGTGGAAGATTCCTCCTGGGAACAGGAATCTGCCCAGCATGAGGATGGCA
GGGATTCCGAAGCCTGCCGCCAGCGCTTCCGGCAATTCTGCTACGGGGATGTGCATGGGC
CTCATGAGGCCTTCAGCCAGCTCTGGGAGCTCTGCTGCCGCTGGCTGCGGCCCGAGCTGC
GTACCAAGGAGCAGATCCTGGAGCTGCTGGTGCTGGAGCAGTTCCTGACAGTGCTGCCAG
GGGAGATCCAGGGCTGGGTGCGTGAGCAGCACCCGGGAAGCGGTGAGGAGGCTGTCGCCT
TGGTGGAGGACCTACAGAAGCAGCCAGTGAAAGCCTGGCGACAGGATGTGCCCTCGGAGG
AGGCGGAACCCGAGGCTGCAGGCCGGGGATCCCAGGCCACGGGGCCTCCCCCGACGGTGG
GGGCACGGAGGCGGCCGTCTGTTCCCCAGGAGCAGCACAGCCATAGCGCCCAGCCTCCTG
CTCTTCTTAAAGAGGGTCGTCCCGGAGAGACGACGGACACCTGCTTTGTCTCTGGGGTCC
ATGGACCTGTGGCATTGGGAGACATCCCATTCTATTTCTCCCGGGAAGAATGGGGCACCC
TGGACCCTGCTCAGCGGGATCTCTTCTGGGACATAAAGCGGGAGAACTCCCGGAACACCA
CCCTGGGTTTTGGGCTCAAAGGCCAAAGTGAGAAGTCCCTGCTGCAGGAGATGGTGCCGG
TGGTGCCAGGCCAGACAGGCAGCGACGTGACTGTGTCCTGGAGCCCCGAGGAGGCTGAGG
CCTGGGAGAGCGAGAACCGGCCGAGGGCGGCCCTGGGCCCAGTGGTGGGCGCGCGACGGG
GGCGGCCACCCACTCGCCGGCGCCAGTTCCGGGACCTGGCAGCCGAGAAGCCGCACAGCT
GCGGGCAGTGTGGAAAGCGCTTCCGCTGGGGCTCGGACCTGGCGCGGCACCAGCGCACGC
ACACGGGCGAGAAGCCACACAAGTGCCCTGAGTGCGACAAGAGCTTCCGCAGCTCCTCGG
ACCTGGTGCGCCACCAAGGCGTGCACACGGGCGAGAAGCCCTTCTCCTGTTCCGAGTGCG
GCAAGAGCTTCAGCCGCAGCGCCTACCTGGCCGACCACCAGCGCATACACACGGGCGAGA
AGCCTTTCGGCTGCAGCGACTGCGGCAAGAGCTTCTCGCTGCGCTCCTACCTGCTGGACC
ATCGGCGTGTGCACACCGGTGAGCGGCCCTTCGGCTGCGGAGAGTGCGACAAGAGCTTCA
AGCAGCGCGCGCACCTCATCGCGCATCAGAGCCTGCACGCCAAGATGGCCCAGCCCGTGG
GGTGAGCAGCTGGCTTGGCCGGAAACCCGGGGGAGGCCCAGCCACGGCACATCCTGCTTT
GTTCACCACTGGGACTCTCCTTCCATCTGTGGCCACCTCCCGGGCTGTCCGAGGGACCCC
AGGGTACCTCACACTCGGAGCTCGCCTGCCCTGCTTGGCTCTGAGGACCTGCCCAGCGCT
CAAAGGGAACGGAAGCCTTCCCCTCCCGCCCCCGATCTTGTCCTCTTTCCCCCTTCTGCG
CCTAGCGTTCCTCTTCCCCTCTAGTTTCCTGGAGCCCCAACACATTCCTGGCAGGGACAG
CAGGGTGGCAAGGACTCAGGTCTAGGTCCCTTCCCAGAAGCCCCCGAGCCTCATTTGACT
GTGTGGCTCTTTGGCCCCCACCCTGTGGGGTGGGTCCATGGGTCAGGCCTCTGCCCTACC
AACCTGTGCCTTTCAGTGGGCGTGGAGGACTGGCCTTGGCCCCCCAGGGGGCTGCTGGAC
TTTGGGAGAGACAGCCCACACCTGTGGGACCGCGGGTCTTAGTCACGGCGGCAGGGGCTT
TCTGGCCCCCTCCCACTCCCGTTTCCAGGCCATGACCACTCTGCCCTGTCCTGGCCATAC
GGACTCGGCCTGCCTTTGCCCTCGGCCTACTTGCCCTAGCATGAGGCTCTGAGAGCCACC
TGCCCACCAATCTGGTGAGGATAATGGTGGCTCCAGCGACAGGAGGCCAACCCTGGAGAC
CAAGAACAGGGCGCCTGGCTGCCATCTTTTCCTCCAGAGGTGGGGCTGCACCAGACTCAG
CACTAGCACTCCATCAGCACTAGCACCTCACTCCATCAGCACTAGCACCTCACTCCATCG
GCCCCGGCACCCTGCTCCATCGGCACTGGCGCCCTGCTCCATCGGCACTAATGCTCCACT
CGGCGCCCCACTCCATCGGCCCCGCTCCATCGGCACTAATGCCCCACTCGGCGCCCCACT
CCATCAGCACTAATGCTCCACTCCATTGGCACTAACGCCCCAACTCCAGCGGCACTAATG
ACCCGCTCCTTTGACATTGGTGCCCCACTCCATCAGCACTAACGCCCTGCTCCATCGGCA
CTGGTGTCCCACTCCATTGTCACTAACGTCCGGCTCCATCGGCACTACCACCCCGCTCCA
TCATCACTATGTCCAGCTCCGTCGGCACTACCACCCTGCTCCATCATCACTACGTCCAGC
TCCAACGGCACTGGTGCCCCATTCCATCGGCACTAACGCCCCGCTCCACCGGCACCAGTG
CCTCGCTCCATTGGCACCAACGCCCAGCTCCACCGGTACTGGCTCCCTGCTCCATCGGCA
CTAACGCCCTGCT
>gi|14777854|gb|XP_036493.1|ZNF213 459 aa linear similar to
Zinc finger protein 213 (Putative transcription factor CR53)
[Homo sapiens].
MAAPLEAQDQAPGEGEGLLIVKVEDSSWEQESAQHEDGRDSEACRQRFRQFCYGDVHGPH
EAFSQLWELCCRWLRPELRTKEQILELLVLEQFLTVLPGEIQGWVREQHPGSGEEAVALV
EDLQKQPVKAWRQDVPSEEAEPEAAGRGSQATGPPPTVGARRRPSVPQEQHSHSAQPPAL
LKEGRPGETTDTCFVSGVHGPVALGDIPFYFSREEWGTLDPAQRDLFWDIKRENSRNTTL
GFGLKGQSEKSLLQEMVPVVPGQTGSDVTVSWSPEEAEAWESENRPRAALGPVVGARRGR
PPTRRRQFRDLAAEKPHSCGQCGKRFRWGSDLARHQRTHTGEKPHKCPECDKSFRSSSDL
VRHQGVHTGEKPFSCSECGKSFSRSAYLADHQRIHTGEKPFGCSDCGKSFSLRSYLLDHR
RVHTGERPFGCGECDKSFKQRAHLIAHQSLHAKMAQPVG
>gi|21536281|gb|NM_003656.3|CAMK1 1501 bp mRNA Homo sapiens
calcium/calmodulin-dependent protein kinase I (CAMK1), mRNA.
GGAGAGAGCCGCCGAGCCGAGCCGAGCCCCAGCTCCAGCAAGAGCGCGGGCGGGTGGCCC
AGGCACGCAGCGGTGAGGACCGCGGCCACAGCTCGGCGCCAACCACCGCGGGCCTCCCAG
CCAGCCCCGCGGCGGGGCAGCCGCAGGAGCCCTGGCTGTGGTCGGGGGGCAGTGGGCCAT
GCTGGGGGCAGTGGAAGGCCCCAGGTGGAAGCAGGCGGAGGACATTAGAGACATCTACGA
CTTCCGAGATGTTCTGGGCACGGGGGCCTTCTCGGAGGTGATCCTGGCAGAAGATAAGAG
GACGCAGAAGCTGGTGGCCATCAAATGCATTGCCAAGGAGGCCCTGGAGGGCAAGGAAGG
CAGCATGGAGAATGAGATTGCTGTCCTGCACAAGATCAAGCACCCCAACATTGTAGCCCT
GGATGACATCTATGAGAGTGGGGGCCACCTCTACCTCATCATGCAGCTGGTGTCGGGTGG
GGAGCTCTTTGACCGTATTGTGGAAAAAGGCTTCTACACGGAGCGGGACGCCAGCCGCCT
CATCTTCCAGGTGCTGGATGCTGTGAAATACCTGCATGACCTGGGCATTGTACACCGGGA
TCTCAAGCCAGAGAATCTGCTGTACTACAGCCTGGATGAAGACTCCAAAATCATGATCTC
CGACTTTGGCCTCTCCAAGATGGAGGACCCGGGCAGTGTGCTCTCCACCGCCTGTGGAAC
TCCGGGATACGTGGCCCCTGAAGTCCTGGCCCAGAAGCCCTACAGCAAGGCTGTGGATTG
CTGGTCCATAGGTGTCATCGCCTACATCTTGCTCTGCGGTTACCCTCCCTTCTATGACGA
GAATGATGCCAAACTCTTTGAACAGATTTTGAAGGCCGAGTACGAGTTTGACTCTCCTTA
CTGGGACGACATCTCTGACTCTGCCAAAGATTTCATCCGGCACTTGATGGAGAAGGACCC
AGAGAAAAGATTCACCTGTGAGCAGGCCTTGCAGCACCCATGGATTGCAGGAGATACAGC
TCTAGATAAGAATATCCACCAGTCGGTGAGTGAGCAGATCAAGAAGAACTTTGCCAAGAG
CAAGTGGAAGCAAGCCTTCAATGCCACGGCTGTGGTGCGGCACATGAGGAAACTGCAGCT
GGGCACCAGCCAGGAGGGGCAGGGGCAGACGGCGAGCCATGGGGAGCTGCTGACACCAGT
GGCTGGGGGGCCGGCAGCTGGCTGTTGCTGTCGAGACTGCTGCGTGGAGCCGGGCACAGA
ACTGTCCCCCACACTGCCCCACCAGCTCTAGGGCCCTGGACCTCGGGTCATGATCCTCTG
CGTGGGAGGGCTTGGGGGCAGCCTGCTCCCCTTCCCTCCCTGAACCGGGAGTTTCTCTGC
CCTGTCCCCTCCTCACCTGCTTCCCTACCACTCCTCACTGCATTTTCCATACAAATGTTT
CTATTTTATTGTTCCTTCTTGTAATAAAGGGAAGATAAAACCAAAAAAAAAAAAAAAAAA
A
>gi|4502553|gb|NP_003647.1|CAMK1 370 aa linear calcium/
calmodulin-dependent protein kinase I [Homo sapiens].
MLGAVEGPRWKQAEDIRDIYDFRDVLGTGAFSEVILAEDKRTQKLVAIKCIAKEALEGKE
GSMENEIAVLHKIKHPNIVALDDIYESGGHLYLIMQLVSGGELFDRIVEKGFYTERDASR
LIFQVLDAVKYLHDLGIVHRDLKPENLLYYSLDEDSKIMISDFGLSKMEDPGSVLSTACG
TPGYVAPEVLAQKPYSKAVDCWSIGVIAYILLCGYPPFYDENDAKLFEQILKAEYEFDSP
YWDDISDSAKDFIRHLMEKDPEKRFTCEQALQHPWIAGDTALDKNIHQSVSEQIKKNFAK
SKWKQAFNATAVVRHMRKLQLGTSQEGQGQTASHGELLTPVAGGPAAGCCCRDCCVEPGT
ELSPTLPHQL
>gi|13186237|gb|NM_023107.1|FGFR1 2590 bp mRNA Homo sapiens
fibroblast growth factor receptor 1 (fms-related tyrosine
kinase 2, Pfeiffer syndrome) (FGFR1), transcript variant 5,
mRNA.
CCTCTTGCGGCCACAGGCGCGGCGTCCTCGGCGGCGGGCGGCAGCTAGCGGGAGCCGGGA
CGCCGGTGCAGCCGCAGCGCGCGGAGGAACCCGGGTGTGCCGGGAGCTGGGCGGCCACGT
CCGGACGGGACCGAGACCCCTCGTAGCGCATTGCGGCGACCTCGCCTTCCCCGGCCGCGA
GCGCGCCGCTGCTTGAAAAGCCGCGGAACCCAAGGACTTTTCTCCGGTCCGAGCTCGGGG
CGCCCCGCAGGCGCACGGTACCCGTGCTGCAGTCGGGCACGCCGCGGCGCCGGGGGCCTC
CGCAGGGCGATGGAGCCGGTCTGCAAGGAAAGTGAGGCGCCGCCGCTGCGTTCTGGAGGA
GGGGGGCACAAGGTCTGGAGACCCCGGGTGGCGGACGGGAGCCCTCCCCCCGCCCCGCCT
CCGGGGCACCAGCTCCGGCTCCATTGTTCCCGCCCGGGCTGGAGGCGCCGAGCACCGAGC
GCCGCCGGGAGTCGAGCGCCGGCCGCGGAGCTCTTGCGACCCCGCCAGGACCCGAACAGA
GCCCGGGGGCGGCGGGCCGGAGCCGGGGACGCGGGCACACGCCCGCTCGCACAAGCCACG
GCGGACTCTCCCGAGGCGGAACCTCCACGCCGAGCGAGGGTCAGTTTGAAAAGGAGGATC
GAGCTCACTGTGGAGTATCCATGGAGATGTGGAGCCTTGTCACCAACCTCTAACTGCAGA
ACTGGGATGTGGAGCTGGAAGTGCCTCCTCTTCTGGGCTGTGCTGGTCACAGCCACACTC
TGCACCGCTAGGCCGTCCCCGACCTTGCCTGAACAAGATGCTCTCCCCTCCTCGGAGGAT
GATGATGATGATGATGACTCCTCTTCAGAGGAGAAAGAAACAGATAACACCAAACCAAAC
CGTATGCCCGTAGCTCCATATTGGACATCCCCAGAAAAGATGGAAAAGAAATTGCATGCA
GTGCCGGCTGCCAAGACAGTGAAGTTCAAATGCCCTTCCAGTGGGACCCCAAACCCCACA
CTGCGCTGGTTGAAAAATGGCAAAGAATTCAAACCTGACCACAGAATTGGAGGCTACAAG
GTCCGTTATGCCACCTGGAGCATCATAATGGACTCTGTGGTGCCCTCTGACAAGGGCAAC
TACACCTGCATTGTGGAGAATGAGTACGGCAGCATCAACCACACATACCAGCTGGATGTC
GTGGAGCGGTCCCCTCACCGGCCCATCCTGCAAGCAGGGTTGCCCGCCAACAAAACAGTG
GCCCTGGGTAGCAACGTGGAGTTCATGTGTAAGGTGTACAGTGACCCGCAGCCGCACATC
CAGTGGCTAAAGCACATCGAGGTGAATGGGAGCAAGATTGGCCCAGACAACCTGCCTTAT
GTCCAGATCTTGAAGGTAATCATGGCACCAGTCTTCGTGGGCCAGTCTACTGGGAAGGAG
ACCACTGTCTCGGGGGCTCAAGTTCCTGTGGGCAGGCTCAGTTGCCCCCGAATGGGATCA
TTCCTCACGCTTCAGGCACACACACTCCATCTCAGTAGGGATCTAGCCACATCCCCCAGG
ACTAGTAACAGAGGTCACAAAGTGGAGGTGAGCTGGGAACAGAGGGCTGCAGGGATGGGT
GGTGCTGGTCTGTAATAAGCTTTGAGAGCAACGTCACTGGGGCTTTGGGGTCAGCTACAC
AAGGAAGGCATTTGGACCCCTGCCTTTTCATTGCCCGAAACCAGAGCCTTTCCACCAAGC
GTTTCCCAGTCTTAGCCCTGTGTTCTGAGTTACGTACGATCTTTCTGGCAAATGGGGTGC
ATGATAAGAGCATCTCTTACGAAGAGTTGGAAAAACAAATGCCATATATAAATTCTAAGC
CATATGAGGACGAGGAGTAATGGCATTTTCTTCCTTTTTCCTCTCACTCCCAGACATTCA
TTGTCCCTGAATGCTCCATTAATCCAGGGAAGGTAATTGCCTAAATCTCCAGTGGATCTC
GCAACAGGAAGGAACCAGAAGCTGGGAAAGTTGTTTACCTCTTTGTCCCAGAGTTAGACC
TCATCCTCCCCTAGCTTAGCTGTCTCAGAGATATACTGGCCCTCCCTTCTCTTCTCTTTG
CTGCTGGTGCTAAAACTGCTCTGTAGGTCATTGGCCACTGTCTCCACTCACAACCCCTGC
TCCAGTCCTGGAGGGAGTGGGTTAAACACAAATAGAACATTCCATTTGAAGCAGTGATTC
TTTTTTTTTTTTTTTTTTTTTAATCAAATGCTTTGGACTTTTGAAGTCCACTTGTTCTGT
ACTTGTAAAAGGGAAAGAAGGCCGGGCGCAGTCGTCACGCCTGTAATCCCAGCACTTTAG
ATCACTTGAGGTCAGGAGTTTGAGACCAGCCCGGCCAACATGGTGAAACCCCATCTCTAC
TAAAAATACAAAAATTAGCTGTGCATAGTGGTTGGCACCTGTAGTCCCAGCTACTCAGGA
GGCTGAGGCAAGCTAACTGCTTGAACCCAGAAGGCAGAGGTTGCAGTGAGCTGAGATCAC
GCCACTGCACTCCAGCCTGGGTGACAGAGTGAGTGAGACTCTGCGTTAAAAAAAAAAAAA
AAAAAAAAAA
>gi|13186238|gb|NP_075595.1|FGFR1 302 aa linear fibroblast
growth factor receptor 1 isoform 5 precursor; fms-related
tyrosine kinase-2; heparin-binding growth factor receptor;
FMS-like tyrosine kinase 2; basic fibroblast growth factor
receptor 1; N-sam tyrosine kinase; FLG protein; protein-
tyrosine kinase; tyrosylprotein kinase; hydroxyaryl-protein
kinase [Homo sapiens].
MWSWKCLLFWAVLVTATLCTARPSPTLPEQDALPSSEDDDDDDDSSSEEKETDNTKPNRM
PVAPYWTSPEKMEKKLHAVPAAKTVKFKCPSSGTPNPTLRWLKNGKEFKPDHRIGGYKVR
YATWSIIMDSVVPSDKGNYTCIVENEYGSINHTYQLDVVERSPHRPILQAGLPANKTVAL
GSNVEFMCKVYSDPQPHIQWLKHIEVNGSKIGPDNLPYVQILKVIMAPVFVGQSTGKETT
VSGAQVPVGRLSCPRMGSFLTLQAHTLHLSRDLATSPRTSNRGHKVEVSWEQRAAGMGGA
GL
>gi|4758007|gb|NM_004071.1|CLK1 1834 bp mRNA Homo sapiens
CDC-like kinase 1 (CLK1), mRNA.
ATTTTTAGATAATCATTAAAGACCACAGAAAATGTAACAGATCCTACTCTTCAAAATAAT
TGCTATTCAGTATTAAAACGAGCAGTCAGCTGCGTGATTCCCGTGATTGCGTTACAAGCT
TTGTCTCCTTCGACTTGGAGTCTTTGTCCAGGACGATGAGACACTCAAAGAGAACTTACT
GTCCTGATTGGGATGACAAGGATTGGGATTATGGAAAATGGAGGAGCAGCAGCAGTCATA
AAAGAAGGAAGAGATCACATAGCAGTGCCCAGGAGAACAAGCGCTGCAAATACAATCACT
CTAAAATGTGTGATAGCCATTATTTGGAAAGCAGGTCTATAAATGAGAAAGATTATCATA
GTCGACGCTACATTGATGAGTACAGAAATGACTACACTCAAGGATGTGAACCTGGACATC
GCCAAAGAGACCATGAAAGCCGGTATCAGAACCATAGTAGCAAGTCTTCTGGTAGAAGTG
GAAGAAGTAGTTATAAAAGCAAACACAGGATTCACCACAGTACTTCACATCGTCGTTCAC
ATGGGAAGAGTCACCGAAGGAAAAGAACCAGGAGTGTAGAGGATGATGAGGAGGGTCACC
TGATCTGTCAGAGTGGAGACGTACTAAGTGCAAGATATGAAATTGTTGATACTTTAGGTG
AAGGAGCTTTTGGAAAAGTTGTGGAGTGCATCGATCATAAAGCGGGAGGTAGACATGTAG
CAGTAAAAATAGTTAAAAATGTGGATAGATACTGTGAAGCTGCTCGCTCAGAAATACAAG
TTCTGGAACATCTGAATACAACAGACCCCAACAGTACTTTCCGCTGTGTCCAGATGTTGG
AATGGTTTGAGCATCATGGTCACATTTGCATTGTTTTTGAACTATTGGGACTTAGTACTT
ACGACTTCATTAAAGAAAATGGTTTTCTACCATTTCGACTGGATCATATCAGAAAGATGG
CATATCAGATATGCAAGTCTGTGAATTTTTTGCACAGTAATAAGTTGACTCACACAGACT
TAAAGCCTGAAAACATCTTATTTGTGCAGTCTGACTACACAGAGGCGTATAATCCCAAAA
TAAAACGTGATGAACGCACCTTAATAAATCCAGATATTAAAGTTGTAGACTTTGGTAGTG
CAACATATGATGACGAACATCACAGTACATTGGTATCTACAAGACATTATAGAGCACCTG
AAGTTATTTTAGCCCTAGGGTGGTCCCAACCATGTGATGTCTGGAGCATAGGATGCATTC
TTATTGAATACTATCTTGGGTTTACCGTATTTCCAACACACGATAGTAAGGAGCATTTAG
CAATGATGGAAAGGATTCTTGGACCTCTACCAAAACATATGATACAGAAAACCAGGAAAC
GTAAATATTTTCACCACGATCGATTAGACTGGGATGAACACAGTTCTGCCGGCAGATATG
TTTCAAGAGCCTGTAAACCTCTGAAGGAATTTATGCTTTCTCAAGATGTTGAACATGAGC
GTCTCTTTGACCTCATTCAGAAAATGTTGGAGTATGATCCAGCCAAAAGAATTACTCTCA
GAGAAGCCTTAAAGCATCCTTTCTTTGACCTTCTGAAGAAAAGTATATAGATCTGTAATT
GGACAGCTCTCTCGAAGAGATCTTACAGACTGTATCAGTCTAATTTTTAAATTTTAAGTT
ATTTTGTACAGCTTTGTAAATTCTTAACATTTTTATATTGCCATGTTTATTTTGTTTGGG
TAATTTGGTTCATTAAGTACATAGCTAAGGTAATGAACATCTTTTTCAGTAATTGTAAAG
TGATTTATTCAGAATAAATTTTTTGTGCTTATGA
>gi|4758008|gb|NP_004062.1|CLK1 484 aa linear CDC-like
kinase 1; protein tyrosine kinase STY [Homo sapiens].
MRHSKRTYCPDWDDKDWDYGKWRSSSSHKRRKRSHSSAQENKRCKYNHSKMCDSHYLESR
SINEKDYHSRRYIDEYRNDYTQGCEPGHRQRDHESRYQNHSSKSSGRSGRSSYKSKHRIH
HSTSHRRSHGKSHRRKRTRSVEDDEEGHLICQSGDVLSARYEIVDTLGEGAFGKVVECID
HKAGGRHVAVKIVKNVDRYCEAARSEIQVLEHLNTTDPNSTFRCVQMLEWFEHHGHICIV
FELLGLSTYDFIKENGFLPFRLDHIRKMAYQICKSVNFLHSNKLTHTDLKPENILFVQSD
YTEAYNPKIKRDERTLINPDIKVVDFGSATYDDEHHSTLVSTRHYPAPEVILALGWSQPC
DVWSIGCILIEYYLGFTVFPTHDSKEHLAMMERILGPLPKHMIQKTRKRKYFHHDRLDWD
EHSSAGRYVSRACKPLKEFMLSQDVEHERLFDLIQKMLEYDPAKRITLREALKHPFFDLL
KKSI
>gi|20127640|gb|NM_025128.2|MUS81 2352 bp mRNA Homo sapiens
MUS81 endonuclease (MUS81), mRNA.
GGCACGAGGGTCTCAAAGGCTGGCTGGAGTGGAGCCAAAGGAAAAGATCGTTAGAGACAG
CGCCCCTGACCAACCACTTAGAGCAGCGCAGGGGTGGGAGGGCGGCCGCAGGCTCTCCTC
TCGTTAGTGCCCCCTGTGTTTGGGGCCCCGTGATCTCAACGGTCCTGCCCTCGGTCTCCC
TCTTCCCCCGCCCCGCCCTGGGCCAGGTGTTCGAATCCCGACTCCAGAACTGGCGGCGTC
CCAGTCCCGCGGGCGTGGAGCGCCGGAGGACCCGCCCTCGGGCTCATGGCGGCCCCGGTC
CGCCTGGGCCGGAAGCGCCCGCTGCCTGCCTGTCCCAACCCGCTCTTCGTTCGCTGGCTG
ACCGAGTGGCGGGACGAGGCGACCCGCAGCAGGCACCGCACGCGCTTCGTATTTCAGAAG
GCGCTGCGTTCCCTCCGACGGTACCCACTGCCGCTGCGCAGCGGGAAGGAAGCTAAGATC
CTACAGCACTTCGGAGACGGGCTCTGCCGGATGCTGGACGAGCGGCTGCAGCGGCACCGA
ACATCGGGCGGTGACCATGCCCCGGACTCACCATCTGGAGAGAACAGTCCAGCCCCGCAG
GGGCGACTTGCGGAAGTCCAGGACTCTTCCATGCCAGTTCCTGCCCAGCCCAAAGCGGGA
GGCTCTGGCAGCTACTGGCCAGCTCGGCACTCAGGAGCCCGAGTGATACTGCTGGTGCTC
TACCGGGAGCACCTGAATCCTAATGGTCACCACTTCTTAACCAAGGAGGAGCTGCTGCAG
AGGTGTGCTCAGAAGTCCCCCAGGGTAGCCCCTGGGAGTGCCCCACCCTGGCCAGCCCTC
CGCTCCCTCCTTCACAGGAACCTGGTCCTCAGGACACACCAGCCAGCCAGGTACTCATTG
ACCCCAGAGGGCCTGGAGCTGGCCCAGAAGTTGGCCGAGTCAGAAGGCCTGAGCTTGCTG
AATGTGGGCATCGGGCCCAAGGAGCCCCCTGGGGAGGAGACAGCAGTGCCAGGAGCAGCT
TCAGCAGAGCTTGCCAGTGAAGCAGGGGTCCAGCAGCAGCCACTGGAGCTGAGGCCTGGA
GAGTACAGGGTGCTGTTGTGTGTGGACATTGGCGAGACCCGGGGGGGCGGGCACAGGCCG
GAGCTGCTCCGAGAGCTACAGCGGCTGCACGTGACCCACACGGTGCGCAAGCTGCACGTT
GGAGATTTTGTGTGGGTGGCTCAGGAGACCAATCCTAGAGACCCAGCAAACCCTGGGGAG
TTGGTACTGGATCACATTGTGGAGCGCAAGCGACTGGATGACCTTTGCAGCAGCATCATC
GACGGCCGCTTCCGGGAGCAGAAGTTCCGACTGAAGCGCTGTGGTCTGGAGCGCCGGGTA
TACCTGGTGGAAGAGCATGGTTCCGTCCACAACCTCAGCCTTCCTGAGAGCACACTGCTG
CAGGCTGTCACCAAGACTCAGGTCATTGATGGCTTTTTTGTGAAGCGCACAGCAGACATT
AAGGAGTCAGCCGCCTACCTGGCCCTCTTGACTCGGGGCCTGCAGAGACTCTACCAGGGC
CACACCCTACGCAGCCGCCCCTGGGGAACCCCTGGGAACCCTGAATCAGGGGCCATGACC
TCTCCAAACCCTCTCTGCTCACTCCTCACCTTCAGTGACTTCAACGCAGGAGCCATCAAG
AATAAGGCCCAGTCGGTGCGAGAAGTGTTTGCCCGGCAGCTGATGCAGGTGCGCGGAGTG
AGTGGGGAGAAGGCAGCAGCCCTGGTGGATCGATACAGCACCCCTGCCAGCCTCCTGGCC
GCCTATGATGCCTGTGCCACCCCCAAGGAACAAGAGACACTGCTGAGCACCATTAAGTGT
GGGCGTCTACAGAGGAATCTGGGGCCTGCTCTGAGCAGGACCTTATCCCAGCTCTACTGC
AGCTACGGCCCCTTGACCTGAGCTTATGCCGTGAAACAGCCCCCAGCCCCCGTCTGTCCC
CCAACCCAGGCTAGCCAGCCTTTTAACAACATCTTTTGGGGTACAATTAGAATCTAAGTG
TTTGCAGCCATATGTGTCATGTAGAAGATGCCTAGCCCTGGGGACCTTGTGAAATACGCA
GGAACCAGGGATACCATCTGGTCCAGTGGTTTTTAAACAAAGCTGCTTAGCACCTGGAAT
TCCCTGGTCAGGGAGATGGAGTCAGTGGGGCATTGCAGCTTGGAATCTATTTTATGTCAC
CAGTTGGTCCTCATCAAATAAAATTTCCTTAGGAGTGCAGAGGGCTCATTGGGAAAATAA
AAATAATAAAAATAAATAAAACTTCCTAAAAGAAAAGATTGAAACCCAAAAAAAAAAAAA
AAAAAAAAAAAA
>gi|13376707|gb|NP_079404.1|MUS82 476 aa linear MUS81
endonuclease [Homo sapiens].
MLDERLQRHRTSGGDHAPDSPSGENSPAPQGRLAEVQDSSMPVPAQPKAGGSGSYWPARH
SGARVILLVLYREHLNPNGHHFLTKEELLQRCAQKSPRVAPGSAPPWPALRSLLHRNLVL
RTHQPARYSLTPEGLELAQKLAESEGLSLLNVGIGPKEPPGEETAVPGAASAELASEAGV
QQQPLELRPGEYRVLLCVDIGETRGGGHRPELLRELQRLHVTHTVRKLHVGDFVWVAQET
NPRDPANPGELVLDHIVERKRLDDLCSSIIDGRFREQKFRLKRCGLERRVYLVEEHGSVH
NLSLPESTLLQAVTNTQVIDGFFVKRTADIKESAAYLALLTRGLQRLYQGHTLRSRPWGT
PGNPESGAMTSPNPLCSLLTFSDFNAGAIKNKAQSVREVFARQLMQVRGVSGEKAAALVD
RYSTPASLLAAYDACATPKEQETLLSTIKCGRLQRNLGPALSRTLSQLYCSYGPLT
>gi|19923239|gb|NM_003376.2|VEGF 3166 bp mRNA Homo sapiens
vascular endothelial growth factor (VEGF), mRNA.
AAGAGCTCCAGAGAGAAGTCGAGGAAGAGAGAGACGGGGTCAGAGAGAGCGCGCGGGCGT
GCGAGCAGCGAAAGCGACAGGGGCAAAGTGAGTGACCTGCTTTTGGGGGTGACCGCCGGA
GCGCGGCGTGAGCCCTCCCCCTTGGGATCCCGCAGCTGACCAGTCGCGCTGACGGACAGA
CAGACAGACACCGCCCCCAGCCCCAGTTACCACCTCCTCCCCGGCCGGCGGCGGACAGTG
GACGCGGCGGCGAGCCGCGGGCAGGGGCCGGAGCCCGCCCCCGGAGGCGGGGTGGAGGGG
GTCGGAGCTCGCGGCGTCGCACTGAAACTTTTCGTCCAACTTCTGGGCTGTTCTCGCTTC
GGAGGAGCCGTGGTCCGCGCGGGGGAAGCCGAGCCGAGCGGAGCCGCGAGAAGTGCTAGC
TCGGGCCGGGAGGAGCCGCAGCCGGAGGAGGGGGAGGAGGAAGAAGAGAAGGAAGAGGAG
AGGGGGCCGCAGTGGCGACTCGGCGCTCGGAAGCCGGGCTCATGGACGGGTGAGGCGGCG
GTGTGCGCAGACAGTGCTCCAGCGCGCGCGCTCCCCAGCCCTGGCCCGGCCTCGGGCCGG
GAGGAAGAGTAGCTCGCCGAGGCGCCGAGGAGAGCGGGCCGCCCCACAGCCCGAGCCGGA
GAGGGACGCGAGCCGCGCGCCCCGGTCGGGCCTCCGAAACCATGAACTTTCTGCTGTCTT
GGGTGCATTGGAGCCTTGCCTTGCTGCTCTACCTCCACCATGCCAAGTGGTCCCAGGCTG
CACCCATGGCAGAAGGAGGAGGGCAGAATCATCACGAAGTGGTGAAGTTCATGGATGTCT
ATCAGCGCAGCTACTGCCATCCAATCGAGACCCTGGTGGACATCTTCCAGGAGTACCCTG
ATGAGATCGAGTACATCTTCAAGCCATCCTGTGTGCCCCTGATGCGATGCGGGGGCTGCT
CCAATGACGAGGGCCTGGAGTGTGTGCCCACTGAGGAGTCCAACATCACCATGCAGATTA
TGCGGATCAAACCTCACCAAGGCCAGCACATAGGAGAGATGAGCTTCCTACAGCACAACA
AATGTGAATGCAGACCAAAGAAAGATAGAGCAAGACAAGAAAATCCCTGTGGGCCTTGCT
CAGAGCGGAGAAAGCATTTGTTTGTACAAGATCCGCAGACGTGTAAATGTTCCTGCAAAA
ACACACACTCGCGTTGCAAGGCGAGGCAGCTTGAGTTAAACGAACGTACTTGCAGATGTG
ACAAGCCGAGGCGGTGAGCCGGGCAGGAGGAAGGAGCCTCCCTCAGGGTTTCGGGAACCA
GATCTCTCTCCAGGAAAGACTGATACAGAACGATCGATACAGAAACCACGCTGCCGCCAC
CACACCATCACCATCGACAGAACAGTCCTTAATCCAGAAACCTGAAATGAAGGAAGAGGA
GACTCTGCGCAGAGCACTTTGGGTCCGGAGGGCGAGACTCCGGCGGAAGCATTCCCGGGC
GGGTGACCCAGCACGGTCCCTCTTGGAATTGGATTCGCCATTTTATTTTTCTTGCTGCTA
AATCACCGAGCCCGGAAGATTAGAGAGTTTTATTTCTGGGATTCCTGTAGACACACCCAC
CCACATACATACATTTATATATATATATATTATATATATATAAAAATAAATATCTCTATT
TTATATATATAAAATATATATATTCTTTTTTTAAATTAACAGTGCTAATGTTATTGGTGT
CTTCACTGGATGTATTTGACTGCTGTGGACTTGAGTTGGGAGGGGAATGTTCCCACTCAG
ATCCTGACAGGGAAGAGGAGGAGATGAGAGACTCTGGCATGATCTTTTTTTTGTCCCACT
TGGTGGGGCCAGGGTCCTCTCCCCTGCCCAAGAATGTGCAAGGCCAGGGCATGGGGGCAA
ATATGACCCAGTTTTGGGAACACCGACAAACCCAGCCCTGGCGCTGAGCCTCTCTACCCC
AGGTCAGACGGACAGAAAGACAAATCACAGGTTCCGGGATGAGGACACCGGCTCTGACCA
GGAGTTTGGGGAGCTTCAGGACATTGCTGTGCTTTGGGGATTCCCTCCACATGCTGCACG
CGCATCTCGCCCCCAGGGGCACTGCCTGGAAGATTCAGGAGCCTGGGCGGCCTTCGCTTA
CTCTCACCTGCTTCTGAGTTGCCCAGGAGGCCACTGGCAGATGTCCCGGCGAAGAGAAGA
GACACATTGTTGGAAGAAGCAGCCCATGACAGCGCCCCTTCCTGGGACTCGCCCTCATCC
TCTTCCTGCTCCCCTTCCTGGGGTGCAGCCTAAAAGGACCTATGTCCTCACACCATTGAA
ACCACTAGTTCTGTCCCCCCAGGAAACCTGGTTGTGTGTGTGTGAGTGGTTGACCTTCCT
CCATCCCCTGGTCCTTCCCTTCCCTTCCCGAGGCACAGAGAGACAGGGCAGGATCCACGT
GCCCATTGTGGAGGCAGAGAAAAGAGAAAGTGTTTTATATACGGTACTTATTTAATATCC
CTTTTTAATTAGAAATTAGAACAGTTAATTTAATTAAAGAGTAGGGTTTTTTTTCAGTAT
TCTTGGTTAATATTTAATTTCAACTATTTATGAGATGTATCTTTTGCTCTCTCTTGCTCT
CTTATTTGTACCGGTTTTTGTATATAAAATTCATGTTTCCAATCTCTCTCTCCCTGATCG
GTGACAGTCACTAGCTTATCTTGAACAGATATTTAATTTTGCTAACACTCAGCTCTGCCC
TCCCCGATCCCCTGGCTCCCCAGCACACATTCCTTTGAAAGAGGGTTTCAATATACATCT
ACATACTATATATATATTGGGCAACTTGTATTTGTGTGTATATATATATATATATGTTTA
TGTATATATGTGATCCTGAAAAAATAAACATCGCTATTCTGTTTTTTATATGTTCAAACC
AAACAAGAAAAAATAGAGAATTCTACATACTAAATCTCTCTCCTTTTTTAATTTTAATAT
TTGTTATCATTTATTTATTGGTGCTACTGTTTATCCGTAATAATTGTGGGGAAAAGATAT
TAACATCACGTCTTTGTCTCTAGTGCAGTTTTTCGAGATATTCCGTAGTACATATTTATT
TTTAAACAACGACAAAGAAATACAGATATATCTTAAAAAAAAAAAA
>gi|19923240|gb|NP_003367.2|VEGF 191 aa linear vascular
endothelial growth factor [Homo sapiens].
MNFLLSWVHWSLALLLYLHHAKWSQAAPMAEGGGQNHHEVVKFMDVYQRSYCHPIETLVD
IFQEYPDEIEYIFKPSCVPLMRCGGCSNDEGLECVPTEESNITMQIMRIKPHQGQEIGEM
SFLQHNKCECRPKKDRARQENPCGPCSERRKHLFVQDPQTCKCSCKNTHSRCKARQLELN
ERTCRCDKPRR
>gi|16306545|gb|NM_033649.1|FGF18 1466 bp mRNA Homo sapiens
fibroblast growth factor 18 (FGF18), transcript variant 2,
mRNA.
CACGGCCGGAGAGACGCGGAGGAGGAGACATGAGCCGGCGGGCGCCCAGACGGAGCGGCC
GTGACGCTTTCGCGCTGCAGCCGCGCGCCCCGACCCCGGAGCGCTGACCCCTGGCCCCAC
GCAGCTCCGCGCCCGGGCCGGAGAGCGCAACTCGGCTTCCAGACCCGCCGCGCATGCTGT
CCCCGGACTGAGCCGGGCAGCCAGCCTCCCACGGACGCCCGGACGGCCGGCCGGCCAGCA
GTGAGCGAGCTTCCCCGCACCGGCCAGGCGCCTCCTGCACAGCGGCTGCCGCCCCGCAGC
CCCTGCGCCAGCCCGGAGGGCGCAGCGCTCGGGAGGAGCCGCGCGGGGCGCTGATGCCGC
AGGGCGCGCCGCGGAGCGCCCCGGAGCAGCAGAGTCTGCAGCAGCAGCAGCCGGCGAGGA
GGGAGCAGCAGCAGCGGCGGCGGCGGCGGCGGCGGCGGCGGAGGCGCCCGGTCCCGGCCG
CGCGGAGCGGACATGTGCAGGCTGGGCTAGGAGCCGCCGCCTCCCTCCCGCCCAGCGATG
TATTCACCGCCCTCCGCCTGCACTTGCCTGTGTTTACACTTCCTGCTGCTGTGCTTCCAG
GTACAGGTGCTGGTTGCCGAGGAGAACGTGGACTTCCGCATCCACGTGGAGAACCAGACG
CGGGCTCGGGACGATGTGAGCCGTAAGCAGCTGCGGCTGTACCAGCTCTACAGCCGGACC
AGTGGGAAACACATCCAGGTCCTGGGCCGCAGGATCAGTGCCCGCGGCGAGGATGGGGAC
AAGTATGCCCAGCTCCTAGTGGAGACAGACACCTTCGGTAGTCAAGTCCGGATCAAGGGC
AAGGAGACGGAATTCTACCTGTGCATGAACCGCAAAGGCAAGCTCGTGGGGAAGCCCGAT
GGCACCAGCAAGGAGTGTGTGTTCATCGAGAAGGTTCTGGAGAACAACTACACGGCCCTG
ATGTCGGCTAAGTACTCCGGCTGGTACGTGGGCTTCACCAAGAAGGGGCGGCCGCGGAAG
GGCCCCAAGACCCGGGAGAACCAGCAGGACGTGCATTTCATGAAGCGCTACCCCAAGGGG
CAGCCGGAGCTTCAGAAGCCCTTCAAGTACACGACGGTGACCAAGAGGTCCCGTCGGATC
CGGCCCACACACCCTGCCTAGGCCACCCCGCCGCGGCCCTCAGGTCGCCCTGGCCACACT
CACACTCCCAGAAAACTGCATCAGAGGAATATTTTTACATGAAAAATAAGGATTTTATTG
TTGACTTGAAACCCCCGATGACAAAAGACTCACGCAAAGGGACTGTAGTCAACCCACAGG
TGCTTGTCTCTCTCTAGGAACAGACAACTCTAAACTCGTCCCCAGAGGAGGACTTGAATG
AGGAAACCAACACTTTGAGAAACCAAAGTCCTTTTTCCCAAAGGTTCTGAAAGGAAAAAA
AAAAAAAAACAAAAAAAAAAAAAAAA
>gi|16306546|gb|NP_387498.1|FGF18 207 aa linear fibroblast
growth factor 18 precursor [Homo sapiens].
MYSAPSACTCLCLHFLLLCFQVQVLVAEENVDFRIHVENQTRARDDVSRKQLRLYQLYSR
TSGKHIQVLGRRISARGEDGDKYAQLLVETDTFGSQVRIKGKETEFYLCMNRKGKLVGKP
DGTSKECVFIEKVLENNYTALMSAKYSGWYVGFTKKGRPRKGPKTRENQQDVHFMKRYPK
GQPELQKPFKYTTVTKRSRRIRPTHPA
>gi|24496766|gb|NM_004712.3|HGS 2926 bp mRNA Homo sapiens
hepatocyte growth factor-regulated tyrosine kinase substrate
(HGS), mRNA.
CGGAAGCGGAAGTCGGGGGGCGCGCCAGCTCGTAGCAGGGGAGCGCCCGCGGCGTCGGGT
TTGGGCTGGAGGTCGCCATGGGGCGAGGCAGCGGCACCTTCGAGCGTCTCCTAGACAAGG
CGACCAGCCAGCTCCTGTTGGAGACAGATTGGGAGTCCATTTTGCAGATCTGCGACCTGA
TCCGCCAAGGGGACACACAAGCAAAATATGCTGTGAATTCCATCAAGAAGAAAGTCAACG
ACAAGAACCCACACGTCGCCTTGTATGCCCTGGAGGTCATGGAATCTGTGGTAAAGAACT
GTGGCCAGACAGTTCATGATGAGGTGGCCAACAAGCAGACCATGGAGGAGCTGAAGGACC
TGCTGAAGAGACAAGTGGAGGTAAACGTCCGTAACAAGATCCTGTACCTGATCCAGGCCT
GGGCGCATGCCTTCCGGAACGAGCCCAAGTACAAGGTGGTCCAGGACACCTACCAGATCA
TGAAGGTGGAGGGGCACGTCTTTCCAGAATTCAAAGAGAGCGATGCCATGTTTGCTGCCG
AGAGAGCCCCAGACTGGGTGGACGCTGAGGAATGCCACCGCTGCAGGGTGCAGTTCGGGG
TGATGACCCGTAAGCACCACTGCCGGGCGTGTGGGCAGATATTCTGTGGAAAGTGTTCTT
CCAAGTACTCCACCATCCCCAAGTTTGGCATCGAGAAGGAGGTGCGCGTGTGTGAGCCCT
GCTACGAGCAGCTGAACAGGAAAGCGGAGGGAAAGGCCACTTCCACCACTGAGCTGCCCC
CCGAGTACCTGACCAGCCCCCTGTCTCAGCAGTCCCAGCTGCCCCCCAAGAGGGACGAGA
CGGCCCTGCAGGAGGAGGAGGAGCTGCAGCTGGCCCTGGCGCTGTCACAGTCAGAGGCGG
AGGAGAAGGAGAGGCTGAGACAGAAGTCCACGTACACTTCGTACCCCAAGGCGGAGCCCA
TGCCCTCGGCCTCCTCAGCGCCCCCCGCCAGCAGCCTGTACTCTTCACCTGTGAACTCGT
CGGCGCCTCTGGCTGAGGACATCGACCCTGAGCTCGCACGGTATCTCAACCGGAACTACT
GGGAGAAGAAGCAGGAGGAGGCTCGCAAGAGCCCCACGCCATCTGCGCCCGTGCCCCTGA
CGGAGCCGGCTGCACAGCCTGGGGAAGGGCACGCAGCCCCCACCAACGTGGTGGAGAACC
CCCTCCCGGAGACAGACTCTCAGCCCATTCCTCCCTCTGGTGGCCCCTTTAGTGAGCCAC
AGTTCCACAATGGCGAGTCTGAGGAGAGCCACGAGCAGTTCCTGAAGGCGCTGCAGAACG
CCGTCACCACCTTCGTGAACCGCATGAAGAGTAACCACATGCGGGGCCGCAGCATCACCA
ATGACTCGGCCGTGCTCTCACTCTTCCAGTCCATCAACGGCATGCACCCGCAGCTGCTGG
AGCTGCTCAACCAGCTGGACGAGCGCAGGCTGTACTATGAGGGGCTGCAGGACAAGCTGG
CACAGATCCGCGATGCCCGGGGGGCGCTGAGTGCCCTGCGCGAAGAGCACCGGGAGAAGC
TTCGCCGGGCAGCCGAGGAGGCAGAGCGCCAGCGCCAGATCCAGCTGGCCCAGAAGCTGG
AGATAATGCGGCAGAAGAAGCAGGAGTACCTGGAGGTGCAGAGGCAGCTGGCCATCCAGC
GCCTGCAGGAGCAGGAGAAGGAGCGGCAGATGCGGCTGGAGCAGCAGAAGCAGACGGTCC
AGATGCGCGCGCAGATGCCCGCCTTCCCCCTGCCCTACGCCCAGCTCCAGGCCATGCCCG
CAGCCGGAGGTGTGCTCTACCAGCCCTCGGGACCAGCCAGCTTCCCCAGCACCTTCAGCC
CTGCCGGCTCGGTGGAGGGCTCCCCAATGCACGGCGTGTACATGAGCCAGCCGGCCCCTG
CCGCTGGCCCCTACCCCAGCATGCCCAGCACTGCGGCTGATCCCAGCATGGTGAGTGCCT
ACATGTACCCAGCAGGGGCCACTGGGGCGCAGGCGGCCCCCCAGGCCCAGGCCGGACCCA
CCGCCAGCCCCGCTTACTCATCCTACCAGCCTACTCCCACAGCGGGCTACCAGAACGTGG
CCTCCCAGGCCCCACAGAGCCTCCCGGCCATCTCTCAGCCTCCGCAGTCCAGCACCATGG
GCTACATGGGGAGCCAGTCAGTCTCCATGGGCTACCAGCCTTACAACATGCAGAATCTCA
TGACCACCCTCCCAAGCCAGGATGCGTCTCTGCCACCCCAGCAGCCCTACATCGCGGGGC
AGCAGCCCATGTACCAGCAGATGGCACCCTCTGGCGGTCCCCCCCAGCAGCAGCCCCCCG
TGGCCCAGCAACCGCAGGCACAGGGGCCGCCGGCACAGGGCAGCGAGGCCCAGCTCATTT
CATTCGACTGACCCAGGCCATGCTCACGTCCGGAGTAACACTACATACAGTTCACCTGAA
ACGCCTCGTCTCTAACTGCCGTCGTCCTGCCTCCCTGTCCTCTACTGCCGGTAGTGTCCC
TTCTCTGCGAGTGAGGGGGGGCCTTCACCCCAAGCCCACCTCCCTTGTCCTCAGCCTACT
GCAGTCCCTGAGTTAGTCTCTGCTTTCTTTCCCCAGGGCTGGGCCATGGGGAGGGAAGGA
CTTTCTCCCAGGGGAAGCCCCCAGCCCTGTGGGTCATGGTCTGTGAGAGGTGGCAGGAAT
GGGGACCCTCACCCCCCAAGCAGCCTGTGCCCTCTGGCCGCACTGTGAGCTGGCTGTGGT
GTCTGGGTGTGGCCTGGGGCTCCCTCTGCAGGGGCCTCTCTCGGCAGCCACAGCCAAGGG
TGGAGGCTTCAGGTCTCCAGCTTCTCTGCTTCTCAGCTGCCATCTCCAGTGCCCCAGAAT
GGTACAGCGATAATAAAATGTATTTCAGAAAAAAAAAAAAAAAAAA
>gi|4758528|gb|NP_004703.1|HGS 777 aa linear hepatocyte
growth factor-regulated tyrosine kinase substrate; human
growth factor-regulated tyrosine kinase substrate [Homo
sapiens].
MGRGSGTFERLLDKATSQLLLETDWESILQICDLIRQGDTQAKYAVNSIKKKVNDKNPHV
ALYALEVMESVVKNCGQTVHDEVANKQTMEELKDLLKRQVEVNVRNKILYLIQAWAHAFR
NEPKYKVVQDTYQIMKVEGHVFPEFKESDAMFAAERAPDWVDAEECHRCRVQFGVMTRKH
HCRACGQIFCGKCSSKYSTIPKFGIEKEVRVCEPCYEQLNRKAEGKATSTTELPPEYLTS
PLSQQSQLPPKRDETALQEEEELQLALALSQSEAEEKERLRQKSTYTSYPKAEPMPSASS
APPASSLYSSPVNSSAPLAEDIDPELARYLNRNYWEKKQEEARKSPTPSAPVPLTEPAAQ
PGEGHAAPTNVVENPLPETDSQPIPPSGGPFSEPQFHNGESEESHEQFLKALQNAVTTFV
NRMKSNHMRGRSITNDSAVLSLFQSINGMHPQLLELLNQLDERRLYYEGLQDKLAQIRDA
RGALSALREEHREKLRRAAEEAERQRQIQLAQKLEIMRQKKQEYLEVQRQLAIQRLQEQE
KERQMRLEQQKQTVQMRAQMPAFPLPYAQLQAMPAAGGVLYQPSGPASFPSTFSPAGSVE
GSPMHGVYMSQPAPAAGPYPSMPSTAADPSMVSAYMYPAGATGAQAAPQAQAGPTASPAY
SSYQPTPTAGYQNVASQAPQSLPAISQPPQSSTMGYMGSQSVSMGYQPYNMQNLMTTLPS
QDASLPPQQPYIAGQQPMYQQMAPSGGPPQQQPPVAQQPQAQGPPAQGSEAQLISFD
>gi|20127435|gb|NM_003821.2|RIPK2 1898 bp mRNA Homo sapiens
receptor-interacting serine-threonine kinase 2 (RIPK2),
mRNA.
GGCACGAGGGTCAGCTCTGGTTCGGAGAAGCAGCGGCTGGCGTGGGCCATCCGGGGAATG
GGCGCCCTCGTGACCTAGTGTTGCGGGGCAAAAAGGGTCTTGCCGGCCTCGCTCGTGCAG
GGGCGTATCTGGGCGCCTGAGCGCGGCGTGGGAGCCTTGGGAGCCGCCGCAGCAGGGGGC
ACACCCGGAACCGGCCTGAGCGCCCGGGACCATGAACGGGGAGGCCATCTGCAGCGCCCT
GCCCACCATTCCCTACCACAAACTCGCCGACCTGCGCTACCTGAGCCGCGGCGCCTCTGG
CACTGTGTCGTCCGCCCGCCACGCAGACTGGCGCGTCCAGGTGGCCGTGAAGCACCTGCA
CATCCACACTCCGCTGCTCGACAGTGAAAGAAAGGATGTCTTAAGAGAAGCTGAAATTTT
ACACAAAGCTAGATTTAGTTACATTCTTCCAATTTTGGGAATTTGCAATGAGCCTGAATT
TTTGGGAATAGTTACTGAATACATGCCAAATGGATCATTAAATGAACTCCTACATAGGAA
AACTGAATATCCTGATGTTGCTTGGCCATTGAGATTTCGCATCCTGCATGAAATTGCCCT
TGGTGTAAATTACCTGCACAATATGACTCCTCCTTTACTTCATCATGACTTGAAGACTCA
GAATATCTTATTGGACAATGAATTTCATGTTAAGATTGCAGATTTTGGTTTATCAAAGTG
GCGCATGATGTCCCTCTCACAGTCACGAAGTAGCAAATCTGCACCAGAAGGAGGGACAAT
TATCTATATGCCACCTGAAAACTATGAACCTGGACAAAAATCAAGGGCCAGTATCAAGCA
CGATATATATAGCTATGCAGTTATCACATGGGAAGTGTTATCCAGAAAACAGCCTTTTGA
AGATGTCACCAATCCTTTGCAGATAATGTATAGTGTGTCACAAGGACATCGACCTGTTAT
TAATGAAGAAAGTTTGCCATATGATATACCTCACCGAGCACGTATGATCTCTCTAATAGA
AAGTGGATGGGCACAAAATCCAGATGAAAGACCATCTTTCTTAAAATGTTTAATAGAACT
TGAACCAGTTTTGAGAACATTTGAAGAGATAACTTTTCTTGAAGCTGTTATTCAGCTAAA
GAAAACAAAGTTACAGAGTGTTTCAAGTGCCATTCACCTATGTGACAAGAAGAAAATGGA
ATTATCTCTGAACATACCTGTAAATCATGGTCCACAAGAGGAATCATGTGGATCCTCTCA
GCTCCATGAAAATAGTGGTTCTCCTGAAACTTCAAGGTCCCTGCCAGCTCCTCAAGACAA
TGATTTTTTATCTAGAAAAGCTCAAGACTGTTATTTTATGAAGCTGCATCACTGTCCTGG
AAATCACAGTTGGGATAGCACCATTTCTGGATCTCAAAGGGCTGCATTCTGTGATCACAA
GACCACTCCATGCTCTTCAGCAATAATAAATCCACTCTCAACTGCAGGAAACTCAGAACG
TCTGCAGCCTGGTATAGCCCAGCAGTGGATCCAGAGCAAAAGGGAAGACATTGTGAACCA
AATGACAGAAGCCTGCCTTAACCAGTCGCTAGATGCCCTTCTGTCCAGGGACTTGATCAT
GAAAGAGGACTATGAACTTGTTAGTACCAAGCCTACAAGGACCTCAAAAGTCAGACAATT
ACTAGACACTACTGACATCCAAGGAGAAGAATTTGCCAAAGTTATAGTACAAAAATTGAA
AGATAACAAACAAATGGGTCTTCAGCCTTACCCGGAAATACTTGTGGTTTCTAGATCACC
ATCTTTAAATTTACTTCAAAATAAAAGCATGTAAGTGACTGTTTTTCAAGAAGAAATGTG
TTTCATAAAAGGATATTTATAAAAAAAAAAAAAAAAAA
>gi|4506537|gb|NP_003812.1|RIPK2 540 aa linear receptor-
interacting serine-threonine kinase 2; receptor interacting
protein 2 [Homo sapiens].
MNGEAICSALPTIPYHKLADLRYLSRGASGTVSSARRADWRVQVAVKHLHIHTPLLDSER
KDVLREAEILHKARFSYILPILGICNEPEFLGIVTEYMPNGSLNELLHRKTEYPDVAWPL
RFRILHEIALGVNYLHNMTPPLLHHDLKTQNILLDNEFHVKIADFGLSKWRMMSLSQSRS
SKSAPEGGTIIYMPPENYEPGQKSRASIKHDIYSYAVITWEVLSRKQPFEDVTNPLQIMY
SVSQGHRPVINEESLPYDIPHRARMISLIESGWAQNPDERPSFLKCLIELEPVLRTFEEI
TFLEAVIQLKKTKLQSVSSAIHLCDKKKMELSLNIPVNHGPQEESCGSSQLHENSGSPET
SRSLPAPQDNDFLSRKAQDCYFMKLHHCPGNHSWDSTISGSQRAAFCDHKTTPCSSAIIN
PLSTAGNSERLQPGIAQQWIQSKREDIVNQMTEACLNQSLDALLSRDLIMKEDYELVSTK
PTRTSKVRQLLDTTDIQGEEFAKVIVQKLKDNKQMGLQPYPEILVVSRSPSLNLLQNKSM
>gi|26051238|gb|NM_021137.3|TNFAIP1 3571 bp mRNA Homo
sapiens tumor necrosis factor, alpha-induced protein 1
(endothelial) (TNFAIP1), mRNA.
CACAGCTTGGGACTGCTGAGGGGCAGGCGGCTGCAGGCTAGGGGCGGCTCGGAGTCCGCT
GGCCACCCAGCTGAGAGGAGAGGCGCCCCCGGGGACGCACTGAGATTATGAGGCTCTGGC
CTCCACTGGCCACTCACTCGTGACCCTTTCCACCACGGCGGAGCCTTCCAAGCCTACCTC
CTGCCGTGTGGTGATCTACCTGCAGCGGGAGATGTCGGGGGACACCTGCCTGTGCCCAGC
CTCAGGGGCCAAGCCCAAGCTCAGTGGCTTCAAGGGAGGAGGGTTGGGCAACAAGTATGT
CCAGCTCAACGTGGGCGGCTCTCTGTACTACACCACTGTGCGGGCCCTGACCCGCCACGA
CACCATGCTCAAGGCCATGTTCAGTGGGCGCATGGAGGTGCTGACCGACAAAGAAGGCTG
GATCCTCATAGACCGTTGTGGAAAGCACTTTGGCACCATTTTGAATTACCTCCGAGATGA
CACCATCACCCTCCCTCAGAACCGGCAAGAAATCAAGGAATTGATGGCTGAAGCAAAGTA
TTACCTCATCCAGGGGCTGGTGAATATGTGCCAGAGTGCCCTGCAGGACAAGAAGGACTC
CTACCAGCCTGTGTGCAACATCCCCATCATCACATCCCTAAAGGAGGAGGAGCGGCTCAT
CGAATCCTCCACCAAGCCCGTGGTGAAGCTGCTGTACAACAGAAGCAACAACAAGTATTC
CTACACCAGCAACTCTGACGACCACCTGCTGAAAAACATCGAGCTGTTTGACAAGCTCTC
CCTGCGCTTCAACGGCCGCGTGCTCTTCATCAAGGATGTCATTGGTGACGAGATCTGCTG
CTGGTCCTTTTATGGCCAGGGCCGTAAGCTGGCAGAGGTGTGCTGTACCTCCATCGTGTA
TGCCACGGAGAAGAAGCAGACCAAGGTGGAATTCCCAGAGGCCCGAATCTATGAGGAGAC
ACTCAACGTCCTACTCTATGAGACTCCCCGCGTCCCCGACAACTCCTTGTTGGAGGCCAC
AAGCCGTAGCCGCAGCCAGGCTTCCCCCAGTGAAGATGAGGAGACCTTTGAACTGCGGGA
CCGTGTCCGCCGCATCCACGTCAAGCGCTACAGCACTTACGATGACCGGCAGCTCGGCCA
CCAGTCTACCCATCGCGACTGACCAGACCCTCAGGGAGTCAGGGCACGGGAGGCCCTATC
TCCCATCCTGTGGAACCCGCCCCATTGGCCACCCCATGCTGCTGCTGCCTGGGTCTCTGC
TCTAGCACCCAGAGGCATGACAGGCCCTGCTCAGAGGTCAGAGGGTCTGGGCAGAGGAGG
GACCACATTCCCCTGCCTTGCCCCTGAGCACTTCTGGAGACTGCGTCCTGTCCTATCTGC
TCACCATCACCCTTCCTGCCCGACGGAGCTGCTTCTGCTCCCTGGGGCATATGGACTGAC
CCACCTCCTGCTGAGAACCTTCCCCTAGGCCCTGTGCAGAAGGGCTACTGCCCCTTAGGC
CTCAGCTGGGGGAAAGGCAGTTCTGGTGCTGTAGAGGCCCTGGTGCAGAAAGTGGGACGT
CTTTTTTCCTAAGGTGTTTAAGCACAGGCTTGATAAGTTTGGTTTTTAAAAAATAATCTA
GGAAATGAATAATTCTAAATCTAGTAATGAGGAAACTGAGCATTTCTTTTGCCCTCCAGG
GTGCCAAGACCCTACATATGACAGAACCCTTGGCCCTTCTCCATGCCTGTGGGATCTGTT
TCTTTAAAGCACTTTGTACTGTTATTCAGGAGGTTGATAATCTCCTTGACCCATGTCTTT
CTACCCTAATCCCCACTTCCCTGCAGAATCAATCTGAGGGAGGGGATAAAGAGGAAGCAA
TAAAAAAAAAACATCCGACAGAGCAGCTCTGGCTTTGCCAGCCTGGCCAGCAGCTCAGAG
TGCACCGAGGAGGGAAGGATGGCTAAGCTGGGACCGGCAGTCCTCACAGGGTGCCTGTGA
GAAAGGACATTTTACCCCCACATCATAGTCACATCACTGACTCCTAGGTCTAGCACGACT
GCTCTTTGTGATTCTCTTGAGTACCCTTGGCTTCCAGCCATGCTGTCCTCACATACGGTA
AAGCCAAAGAGCTGTCACATGGGCCAGAAACATGAGCCACGGCAGGAAGACCGTGGAGCC
CGTGGGCACTGCATGGTGTTGGCTGGCATGCCCATCAGCTGAGGACAGCAAACTCCCAGC
AGCCCCTACAGAGGTGGCACATGCTTGGCCACACATCTACTCCCTGCCCACACCATCTAT
GCTCTTGGTTGGTGCTGGCTGGGATGGCGGTTCTGCCCAGTGGTGTCTCTGAGCGCGGGA
TGACAGGAGCAACCGAAGCACCCTGAAGGCCTTCACTCCTTGTTGGGTAACTCAGCCATG
GAGATGCCAAGCACTAGCCAGGAGGTGAGTTCCTCTTTAGGGCTTTGGTTTTCATTCCTT
TTTGTTTGGCTTGGCCAAACCAGAATTCAGCTTATCTGAATTATTTTCCAAAGGAATGCT
GTCAGGGAGGGACTGTTCTGCCAGCCTAACAAAGCAACGTAGCCACGTATAGTACCCACT
TTCTGCTCTTTGGAGAGAACACAGGTTATCAAGTTCATCTCTCTTGACTACTCTTATGAT
AGCTGATGCCACAGAGCCTATGGGCAAATGCCAGACCCAGGGTTAGACACAAGGACCTGA
AGTGACATGACGGCGGGACAGGGGAAATGTGACTTTCTAATTAGGCATTTTATGTTAGTC
ACAGTCTTGAATGTATAAACAGCACTAAGACTCTCAGGTCAGGTACCTTGGTGATCAGCT
ACTAGTTCTTCCAGCCCTCATTGAGGTAACAAGATAAAGACAAATCCACTTCTTTGGCCA
AATTCAGGCTTTGGCTTTATGACTTTCCCACAGAGACTGGAATGCGTCAGCCTGAGACCA
CTGGCCTATTTTCTCAGCTGCCCTCTTGAGGTCCTTTAACACTCAAATTCCCAGCTCCCC
ACTGAGGTGTTGTGATGCTTGCCTTTTGACCTCCCCATCCCCTTTAGTCCCTGCTTACTA
CTTTGACATTCACATCCTCAGTGTCTCAGTCTTTTTTGCCGAGAAAGCACAGTAGTCTGG
GACTGGGCATTTATCTTCTCTGACTGAAAATCTCTCCTTGGTCTTAAGGAAAATACTAAC
ATTGAACTCACTGACATGATCTTAGCTTCTTTAATCAGACTTTGTGACTTAAAAGTTTGG
GGGTTTTCTTTGAAAGTTTCCAGCCCTATTCAGAAAGCAACTCTTGGCTGTGTGCATTTT
TCAACTCCAAGCAGCCCAGGGGTAAGTAAACAAAGTATGGATGAAGGTCAGATTTTCTTG
TCAGTTTCTGAGAAACCTGGCAGCCTGCTGTTAACAACACAGGCCAGTATTGGGTTTTAT
TGAATTTGGTATGTGACCAAGGTCGGCCTAAAGGATGGCGCAGGTCCTGGGCAGGAAAGA
ATTTTTCCTTTATCACATAACTGTAATATTTGGTTGCTCAGCATAAGTGATGGAAGCAAA
CACTAATTTCTAATAAAATTGTGTTAAACTC
>gi|10863937|gb|NP_066960.1|TNFAIP1 316 aa linear tumor
necrosis factor, alpha-induced protein 1 [Homo sapiens].
MSGDTCLCPASGAKPKLSGFKGGGLGNKYVQLNVGGSLYYTTVRALTRHDTMLKAMFSGR
MEVLTDKEGWILIDRCGKHFGTILNYLRDDTITLPQNRQEIKELMAEAKYYLIQGLVNMC
QSALQDKKDSYQPVCNIPIITSLKEEERLIESSTKPVVKLLYNRSNNKYSYTSNSDDHLL
KNIELFDKLSLRFNGRVLFIKDVIGDEICCWSFYGQGRKLAEVCCTSIVYATEKKQTKVE
FPEARIYEETLNVLLYETPRVPDNSLLEATSRSRSQASPSEDEETPELRDRVRRIHVKRY
STYDDRQLGHQSTHRD
>gi|27597077|gb|NM_006293.2|TYRO3 3949 bp mRNA Homo sapiens
TYRO3 protein tyrosine kinase (TYRO3), mRNA.
GCGGTGGCGCGGGAGCGGCCCCGGGGACCCCGCGCTGCTGACGGCGGCGACCGCGGCCGG
AGGCGGGCGCGGGTCTCGGAGGCGGTCGCCTCAGCACCGCCCCACGGGCGGCCCCAGCCC
CTCCCGCAGCCCTCCTCCCTCCCGCTCCCTTCCCGCCGCCTCCTCCCCGCCCTCCTCCCT
CCTCGCTCGCGGGCCGGGCCCGGCATGGTGCGGCGTCGCCGCCGATGGCGCTGAGGCGGA
GCATGGGGCGGCCGGGGCTCCCGCCGCTGCCGCTGCCGCCGCCACCGCGGCTCGGGCTGC
TGCTGGCGGCTCTGGCTTCTCTGCTGCTCCCGGAGTCCGCCGCCGCAGGTCTGAAGCTCA
TGGGAGCCCCGGTGAAGCTGACAGTGTCTCAGGGGCAGCCGGTGAAGCTCAACTGCAGTG
TGGAGGGGATGGAGGAGCCTGACATCCAGTGGGTGAAGGATGGGGCTGTGGTCCAGAACT
TGGACCAGTTGTACATCCCAGTCAGCGAGCAGCACTGGATCGGCTTCCTCAGCCTGAAGT
CAGTGGAGCGCTCTGACGCCGGCCGGTACTGGTGCCAGGTGGAGGATGGGGGTGAAACCG
AGATCTCCCAGCCAGTGTGGCTCACGGTAGAAGGTGTGCCATTTTTCACAGTGGAGCCAA
AAGATCTGGCAGTGCCACCCAATGCCCCTTTCCAACTGTCTTGTGAGGCTGTGGGTCCCC
CTGAACCTGTTACCATTGTCTGGTGGAGAGGAACTACGAAGATCGGGGGACCCGCTCCCT
CTCCATCTGTTTTAAATGTAACAGGGGTGACCCAGAGCACCATGTTTTCCTGTGAAGCTC
ACAACCTAAAAGGCCTGGCCTCTTCTCGCACAGCCACTGTTCACCTTCAAGCACTGCCTG
CAGCCCCCTTCAACATCACCGTGACAAAGCTTTCCAGCAGCAACGCTAGTGTGGCCTGGA
TGCCAGGTGCTGATGGCCGAGCTCTGCTACAGTCCTGTACAGTTCAGGTGACACAGGCCC
CAGGAGGCTGGGAAGTCCTGGCTGTTGTGGTCCCTGTGCCCCCCTTTACCTGCCTGCTCC
GGGACCTGGTGCCTGCCACCAACTACAGCCTCAGGGTGCGCTGTGCCAATGCCTTGGGGC
CCTCTCCCTATGCTGACTGGGTGCCCTTTCAGACCAAGGGTCTAGCCCCAGCCAGCGCTC
CCCAAAACCTCCATGCCATCCGCACAGATTCAGGCCTCATCTTGGAGTGGGAAGAAGTGA
TCCCCGAGGCCCCTTTGGAAGGCCCCCTGGGACCCTACAAACTGTCCTGGGTTCAAGACA
ATGGAACCCAGGATGAGCTGACAGTGGAGGGGACCAGGGCCAATTTGACAGGCTGGGATC
CCCAAAAGGACCTGATCGTACGTGTGTGCGTCTCCAATGCAGTTGGCTGTGGACCCTGGA
GTCAGCCACTGGTGGTCTCTTCTCATGACCGTGCAGGCCAGCAGGGCCCTCCTCACAGCC
GCACATCCTGGGTACCTGTGGTCCTTGGTGTGCTAACGGCCCTGGTGACGGCTGCTGCCC
TGGCCCTCATCCTGCTTCGAAAGAGACGGAAAGAGACGCGGTTTGGGCAAGCCTTTGACA
GTGTCATGGCCCGGGGAGAGCCAGCCGTTCACTTCCGGGCAGCCCGGTCCTTCAATCGAG
AAAGGCCCGAGCGCATCGAGGCCACATTGGACAGCTTGGGCATCAGCGATGAACTAAAGG
AAAAACTGGAGGATGTGCTCATCCCAGAGCAGCAGTTCACCCTGGGCCGGATGTTGGGCA
AAGGAGAGTTTGGTTCAGTGCGGGAGGCCCAGCTGAAGCAAGAGGATGGCTCCTTTGTGA
AAGTGGCTGTGAAGATGCTGAAAGCTGACATCATTGCCTCAAGCGACATTGAAGAGTTCC
TCAGGGAAGCAGCTTGCATGAAGGAGTTTGACCATCCACACGTGGCCAAACTTGTTGGGG
TAAGCCTCCGGAGCAGGGCTAAAGGCCGTCTCCCCATCCCCATGGTCATCTTGCCCTTCA
TGAAGCATGGGGACCTGCATGCCTTCCTGCTCGCCTCCCGGATTGGGGAGAACCCCTTTA
ACCTACCCCTCCAGACCCTGATCCGGTTCATGGTGGACATTGCCTGCGGCATGGAGTACC
TGAGCTCTCGGAACTTCATCCACCGAGACCTGGCTGCTCGGAATTGCATGCTGGCAGAGG
ACATGACAGTGTGTGTGGCTGACTTCGGACTCTCCCGGAAGATCTACAGTGGGGACTACT
ATCGTCAAGGCTGTGCCTCCAAACTGCCTGTCAAGTGGCTGGCCCTGGAGAGCCTGGCCG
ACAACCTGTATACTGTGCAGAGTGACGTGTGGGCGTTCGGGGTGACCATGTGGGAGATCA
TGACACGTGGGCAGACGCCATATGCTGGCATCGAAAACGCTGAGATTTACAACTACCTCA
TTGGCGGGAACCGCCTGAAACAGCCTCCGGAGTGTATGGAGGACGTGTATGATCTCATGT
ACCAGTGCTGGAGTGCTGACCCCAAGCAGCGCCCGAGCTTTACTTGTCTGCGAATGGAAC
TGGAGAACATCTTGGGCCAGCTGTCTGTGCTATCTGCCAGCCAGGACCCCTTATACATCA
ACATCGAGAGAGCTGAGGAGCCCACTGCGGGAGGCAGCCTGGAGCTACCTGGCAGGGATC
AGCCCTACAGTGGGGCTGGGGATGGCAGTGGCATGGGGGCAGTGGGTGGCACTCCCAGTG
ACTGTCGGTACATACTCACCCCCGGAGGGCTGGCTGAGCAGCCAGGGCAGGCAGAGCACC
AGCCAGAGAGTCCCCTCAATGAGACACAGAGGCTTTTGCTGCTGCAGCAAGGGCTACTGC
CACACAGTAGCTGTTAGCCCACAGGCAGAGGGCATCGGGGCCATTTGGCCGGCTCTGGTG
GCCACTGAGCTGGCTGACTAAGCCCCGTCTGACCCCAGCCCAGACAGCAAGGTGTGGAGG
CTCCTGTGGTAGTCCTCCCAAGCTGTGCTGGGAAGCCCGGACTGACCAAATCACCCAATC
CCAGTTCTTCCTGCAACCACTCTGTGGCCAGCCTGGCATCAGTTTAGGCCTTGGCTTGAT
GGAAGTGGGCCAGTCCTGGTTGTCTGAACCCAGGCAGCTGGCAGGAGTGGGGTGGTTATG
TTTCCATGGTTACCATGGGTGTGGATGGCAGTGTGGGGAGGGCAGGTCCAGCTCTGTGGG
CCCTACCCTCCTGCTGAGCTGCCCCTGCTGCTTAAGTGCATGCATTGAGCTGCCTCCAGC
CTGGTGGCCCAGCTATTACCACACTTGGGGTTTAAATATCCAGGTGTGCCCCTCCAAGTC
ACAAAGAGATGTCCTTGTAATATTCCCTTTTAGGTGAGGGTTGGTAAGGGGTTGGTATCT
CAGGTCTGAATCTTCACCATCTTTCTGATTCCGCACCCTGCCTACGCCAGGAGAAGTTGA
GGGGAGCATGCTTCCCTGCAGCTGACCGGGTCACACAAAGGCATGCTGGAGTACCCAGCC
TATCAGGTGCCCCTCTTCCAAAGGCAGCGTGCCGAGCCAGCAAGAGGAAGGGGTGCTGTG
AGGCTTGCCCAGGAGCAAGTGAGGCCGGAGAGGAGTTCAGGAACCCTTCTCCATACCCAC
AATCTGAGCACGCTACCAAATCTCAAAATATCCTAAGACTAACAAAGGCAGCTGTGTCTG
AGCCCAACCCTTCTAAACGGTGACCTTTAGTGCCAACTTCCCCTCTAACTGGACAGCCTC
TTCTGTCCCAAGTCTCCAGAGAGAAATCAGGCCTGATGAGGGGGAATTCCTGGAACCTGG
ACCCCAGCCTTGGTGGGGGAGCCTCTGGAATGCATGGGGCGGGTCCTAGCTGTTAGGGAC
ATTTCCAAGCTGTTAGTTGCTGTTTAAAATAGAAATAAAATTGAAGACT
>gi|27597078|gb|NP_006284.2|TYRO3 890 aa linear TYRO3
protein tyrosine kinase; Brt; Dtk, Sky; Tif; Tyro3 protein
tyrosine kinase (sea-related receptor tyrosine kinase);
tyrosine-protein kinase receptor TYRO3 precursor [Homo
sapiens].
MALRRSMGRPGLPPLPLPPPPRLGLLLAALASLLLPESAAAGLKLMGAPVKLTVSQGQPV
KLNCSVEGMEEPDIQWVKDGAVVQNLDQLYIPVSEQHWIGFLSLKSVERSDAGRYWCQVE
DGGETEISQPVWLTVEGVPFFTVEPKDLAVPPNAPFQLSCEAVGPPEPVTIVWWRGTTKI
GGPAPSPSVLNVTGVTQSTMFSCEAHNLKGLASSRTATVHLQALPAAPFNITVTKLSSSN
ASVAWMPGADGRALLQSCTVQVTQAPGGWEVLAVVVPVPPFTCLLRDLVPATNYSLRVRC
ANALGPSPYADWVPFQTKGLAPASAPQNLHAIRTDSGLILEWEEVIPEAPLEGPLGPYKL
SWVQDNGTQDELTVEGTRANLTGWDPQKDLIVRVCVSNAVGCGPWSQPLVVSSHDRAGQQ
GPPHSRTSWVPVVLGVLTALVTAAALALILLRKRRKETRFGQAFDSVMARGEPAVHFRAA
RSFNRERPERIEATLDSLGISDELKEKLEDVLIPEQQFTLGRMLGKGEFGSVREAQLKQE
DGSFVKVAVKMLKADIIASSDIEEFLREAACMKEFDHPHVAKLVGVSLRSRAKGRLPIPM
VILPFMKHGDLHAFLLASRIGENPFNLPLQTLIRFMVDIACGMEYLSSRNFIHRDLAARN
CMLAEDMTVCVADFGLSRKIYSGDYYRQGCASKLPVKWLALESLADNLYTVQSDVWAFGV
TMWEIMTRGQTPYAGIENAEIYNYLIGGNRLKQPPECMEDVYDLMYQCWSADPKQRPSFT
CLRMELENILGQLSVLSASQDPLYINIERAEEPTAGGSLELPGRDQPYSGAGDGSGMGAV
GGTPSDCRYILTPGGLAEQPGQAEHQPESPLNETQRLLLLQQGLLPHSSC
>gi|4502884|gb|NM_003992.1|CLK3 1762 bp mRNA Homo sapiens
CDC-like kinase 3 (CLK3), transcript variant phclk3, mRNA.
TGGGGCACTGGTACCTCCAGGACCTGGAGTGTACTGGAAGAAATGGTGCAGTCCAGATGC
ATCACTGTAAGCGATACCGCTCCCCTGAACCAGACCCGTACCTGAGCTACCGATGGAAGA
GGAGGAGGTCCTACAGTCGGGAACATGAAGGGAGACTGCGATACCCGTCCCGAAGGGAGC
CTCCCCCACGAAGATCTCGGTCCAGAAGCCATGACCGCCTGCCCTACCAGAGGAGGTACC
GGGAGCGCCGTGACAGCGATACATACCGGTGTGAAGAGCGGAGCCCATCCTTTGGAGAGG
ACTACTATGGACCTTCACGTTCTCGTCATCGTCGGCGATCGCGGGAGAGGGGGCCATACC
GGACCCGCAAGCATGCCCACCACTGCCACAAACGCCGCACCAGGTCTTGTAGCAGCGCCT
CCTCGAGAAGCCAACAGAGCAGTAAGCGCACAGGCCGGAGTGTGGAAGATGACAAGGAGG
GTCACCTGGTGTGCCGGATCGGCGATTGGCTCCAAGAGCGATATGAGATTGTGGGGAACC
TGGGTGAAGGCACCTTTGGCAAGGTGGTGGAGTGCTTGGACCATGCCAGAGGGAAGTCTC
AGGTTGCCCTGAAGATCATCCGCAACGTGGGCAAGTACCGGGAGGCTGCCCGGCTAGAAA
TCAACGTGCTCAAAAAAATCAAGGAGAAGGACAAAGAAAACAAGTTCCTGTGTGTCTTGA
TGTCTGACTGGTTCAACTTCCACGGTCACATGTGCATCGCCTTTGAGCTCCTGGGCAAGA
ACACCTTTGAGTTCCTGAAGGAGAATAACTTCCAGCCTTACCCCCTACCACATGTCCGGC
ACATGGCCTACCAGCTCTGCCACGCCCTTAGATTTCTGCATGAGAATCAGCTGACCCATA
CAGACTTGAAACCTGAGAACATCCTGTTTGTGAATTCTGAGTTTGAAACCCTCTACAATG
AGCACAAGAGCTGTGAGGAGAAGTCAGTGAAGAACACCAGCATCCGAGTGGCTGACTTTG
GCAGTGCCACATTTGACCATGAGCACCACACCACCATTGTGGCCACCCGTCACTATCGCC
CGCCTGAGGTGATCCTTGAGCTGGGCTGGGCACAGCCCTGTGACGTCTGGAGCATTGGCT
GCATTCTCTTTGAGTACTACCGGGGCTTCACACTCTTCCAGACCCACGAAAACCGAGAGC
ACCTGGTGATGATGGAGAAGATCCTAGGGCCCATCCCATCACACATGATCCACCGTACCA
GGAAGCAGAAATATTTCTACAAAGGGGGCCTAGTTTGGGATGAGAACAGCTCTGACGGCC
GGTATGTGAAGGAGAACTGCAAACCTCTGAAGAGTTACATGCTCCAAGACTCCCTGGAGC
ACGTGCAGCTGTTTGACCTGATGAGGAGGATGTTAGAATTTGACCCTGCCCAGCGCATCA
CACTGGCCGAGGCCCTGCTGCACCCCTTCTTTGCTGGCCTGACCCCTGAGGAGCGGTCCT
TCCACACCAGCCGCAACCCAAGCAGATGACAGGCACAGGCCACCGCATGAGGAGATGGAG
GGCGGGACTGGGCCGCCCAGCCCCTTGACTCCAGCCTCGACCGCCAGCCCCAGGCCAGAG
CCACCCAATGAACAGTGCAATGTGAAGGAAGGCAGGAGCCTGCAGGGGAGCAGACTTGGT
GCCCAGCTGCCAGAAAGCACAGATTTGACCCAAGCTATTTATATGTTATAAAGTTATAAT
AAAGTGTTTCTTACTGTTTGTA
>gi|4502885|gb|NP_003983.1|CLK3 490 aa linear CDC-like
kinase 3 isoform hclk3 [Homo sapiens].
MHHCKRYRSPEPDPYLSYRWKRRRSYSREHEGRLRYPSRREPPPRRSRSRSHDRLPYQRR
YRERRDSDTYRCEERSPSFGEDYYGPSRSRHRRRSRERGPYRTRKHAHHCHKRRTRSCSS
ASSRSQQSSKRTGRSVEDDKEGHLVCRIGDWLQBRYEIVGNLGEGTFGKVVECLDHARGK
SQVALKIIRNVGKYREAARLEINVLKKIKEKDKENKFLCVLMSDWFNFHGHMCIAFELLG
KWTFEFLKENNFQPYPLPHVRHMAYQLCHALRFLHENQLTHTDLKPENILFVNSEFETLY
NEHKSCEEKSVKNTSIRVADFGSATFDHEHHTTIVATRHYRPPEVILELGWAQPCDVWSI
GCILFEYYRGFTLFQTHENREHLVMMEKILGPIPSHMIHRTRKQKYFYKGGLVWDENSSD
GRYVKENCKPLKSYMLQDSLEHVQLFDLMRRMLEFDPAQRITLAEALLHPFFAGLTPEER
SFHTSRNPSR
>gi|9910121|gb|NM_020249.1|ADAMTS9 3674 bp mRNA Homo sapiens
a disintegrin-like and metalloprotease (reprolysin type)
with thrombospondin type 1 motif, 9 (ADAMTS9), mRNA.
GCGGGAAGCACCATGCAGTTTGTATCCTGGGCCACACTGCTAACGCTCCTGGTGCGGGAC
CTGGCCGAGATGGGGAGCCCAGACGCCGCGGCGGCCGTACGCAAGGACAGGCTGCACCCG
AGGCAAGTGAAATTATTAGAGACCCTGGGCGAATACGAAATCGTGTCTCCCATCCGAGTG
AACGCTCTCGGAGAACCCTTTCCCACGAACGTCCACTTCAAAAGAACGCGACGGAGCATT
AACTCTGCCACTGACCCCTGGCCTGCCTTCGCCTCCTCCTCTTCCTCCTCTACCTCCTCC
CAGGCGCATTACCGCCTCTCTGCCTTCGGCCAGCAGTTTCTATTTAATCTCACCGCCAAT
GCCGGATTTATCGCTCCACTGTTCACTGTCACCCTCCTCGGGACGCCCGGGGTGAATCAG
ACCAAGTTTTATTCCGAAGAGGAAGCGGAACTCAAGCACTGTTTCTACAAAGGCTATGTC
AATACCAACTCCGAGCACACGGCCGTCATCAGCCTCTGCTCAGGAATGCTGGGCACATTC
CGGTCTCATGATGGGGATTATTTTATTGAACCACTACAGTCTATGGATGAACAAGAAGAT
GAAGAGGAACAAAACAAACCCCACATCATTTATAGGCGCAGCGCCCCCCAGAGAGAGCCC
TCAACAGGAAGGCATGCATGTGACACCTCAGAACACAAAAATAGGCACAGTAAAGACAAG
AAGAAAACCAGAGCAAGAAAATGGGGAGAAAGGATTAACCTGGCTGGTGACGTAGCAGCA
TTAAACAGCGGCTTAGCAACAGAGGCATTTTCTGCTTATGGTAATAAGACGGACAACACA
AGAGAAAAGAGGACCCACAGAAGGACAAAACGTTTTTTATCCTATCCACGGTTTGTAGAA
GTCTTGGTGGTGGCAGACAACAGAATGGTTTCATACCATGGAGAAAACCTTCAACACTAT
ATTTTAACTTTAATGTCAATTGTAGCCTCTATCTATAAAGACCCAAGTATTGGAAATTTA
ATTAATATTGTTATTGTGAACTTAATTGTGATTCATAATGAACAGGATGGGCCTTCCATA
TCTTTTAATGCTCAGACAACATTAAAAAACCTTTGCCAGTGGCAGCATTCGAAGAACAGT
CCAGGTGGAATCCATCATGATACTGCTGTTCTCTTAACAAGACAGGATATCTGCAGAGCT
CACGACAAATGTGATACCTTAGGCCTGGCTGAACTGGGAACCATTTGTGATCCCTATAGA
AGCTGTTCTATTAGTGAAGATAGTGGATTGAGTACAGCTTTTACGATCGCCCATGAGCTG
GGCCATGTGTTTAACATGCCTCATGATGACAACAACAAATGTAAAGAAGAAGGAGTTAAG
AGTCCCCAGCATGTCATGGCTCCAACACTGAACTTCTACACCAACCCCTGGATGTGGTCA
AAGTGTAGTCGAAAATATATCACTGAGTTTTTAGACACTGGTTATGGCGAGTGTTTGCTT
AACGAACCTGAATCCAGACCCTACCCTTTGCCTGTCCAACTGCCAGGCATCCTTTACAAC
GTGAATAAACAATGTGAATTGATTTTTGGACCAGGTTCTCAGGTGTGCCCATATATGATG
CAGTGCAGACGGCTCTGGTGCAATAACGTCAATGGAGTACACAAAGGCTGCCGGACTCAG
CACACACCCTGGGCCGATGGGACGGAGTGCGAGCCTGGAAAGCACTGCAAGTATGGATTT
TGTGTTCCCAAAGAAATGGATGTCCCCGTGACAGATGGATCCTGGGGAAGTTGGAGTCCC
TTTGGAACCTGCTCCAGAACATGTGGAGGGGGCATCAAAACAGCCATTCGAGAGTGCAAC
AGACCAGAACCAAAAAATGGTGGAAAATACTGTGTAGGACGTAGAATGAAATTTAAGTCC
TGCAACACGGAGCCATGTCTCAAGCAGAAGCGAGACTTCCGAGATGAACAGTGTGCTCAC
TTTGACGGGAAGCATTTTAACATCAACGGTCTGCTTCCCAATGTGCGCTGGGTCCCTAAA
TACAGTGGAATTCTGATGAAGGACCGGTGCAAGTTGTTCTGCAGAGTGGCAGGGAACACA
GCCTACTATCAGCTTCGAGACAGAGTGATAGATGGAACTCCTTGTGGCCAGGACACAAAT
GATATCTGTGTCCAGGGCCTTTGCCGGCAAGCTGGATGCGATCATGTTTTAAACTCAAAA
GCCCGGAGAGATAAATGTGGGGTTTGTGGTGGCGATAATTCTTCATGCAAAACAGTGGCA
GGAACATTTAATACAGTACATTATGGTTACAATACTGTGGTCCGAATTCCAGCTGGTGCT
ACCAATATTGATGTGCGGCAGCACAGTTTCTCAGGGGAAACAGACGATGACAACTACTTA
GCTTTATCAAGCAGTAAAGGTGAATTCTTGCTAAATGGAAACTTTGTTGTCACAATGGCC
AAAAGGGAAATTCGCATTGGGAATGCTGTGGTAGAGTACAGTGGGTCCGAGACTGCCGTA
GAAAGAATTAACTCAACAGATCGCATTGAGCAAGAACTTTTGCTTCAGGTTTTGTCGGTG
GGAAAGTTGTACAACCCCGATGTACGCTATTCTTTCAATATTCCAATTGAAGATAAACCT
CAGCAGTTTTACTGGAACAGTCATGGGCCATGGCAAGCATGCAGTAAACCCTGCCAAGGG
GAACGGAAACGAAAACTTGTTTGCACCAGGGAATCTGATCAGCTTACTGTTTCTGATCAA
AGATGCGATCGGCTGCCCCAGCCTGGACACATTACTGAACCCTGTGGTACAGACTGTGAC
CTGAGGTGGCATGTTGCCAGCAGGAGTGAATGTAGTGCCCAGTGTGGCTTGGGTTACCGC
ACATTGGACATCTACTGTGCCAAATATAGCAGGCTGGATGGGAAGACTGAGAAGGTTGAT
GATGGTTTTTGCAGCAGCCATCCCAAACCAAGCAACCGTGAAAAATGCTCAGGGGAATGT
AACACGGGTGGCTGGCGCTATTCTGCCTGGACTGAATGTTCAAAAAGCTGTGACGGTGGG
ACCCAGAGGAGAAGGGCTATTTGTGTCAATACCCGAAATGATGTACTGGATGACAGCAAA
TGCACACATCAAGAGAAAGTTACCATTCAGAGGTGCAGTGAGTTCCCTTGTCCACAGTGG
AAATCTGGAGACTGGTCAGAGGTAAGATGGGAGGGCTGTTATTTCCCCTAGGTCATCTCT
TACATTCTAGTTCTGGTGCTCTCTATCTGTTTAAGACAAACCCTTGTGCACCTTTCTCCC
ACCTCTCCCTTTCTCCCTTGTCTCCCTTGAGAAAACAACTCCAGTTCTCTGCCTGCACCA
TGACTGTCGTACTGGATGTAACTAGTCTACCAGTGACCTCAGGGCACTTTGGGCTTGGCT
AGATCACTCACTGTTGTAGCTTCTGTTGTGATTTTGAAGTTGCAGTCCATCACCTTCCCT
CCTCTTTGAGCCCTAGCTAAGTCACTGAAAGGAAATCATGGATTTATTAATCATAAAGCT
ATACTAGCTCACATCTGAAGTCAACATGAAGTTTCCTACTTCCTTGTCTTTGAAATAAGA
GAATTAGACCCCAGGGAGTGACCTCTCTGACTTACCCATCCAACTGCCCAAAAAAAAAAA
AAAAAAAAAAAAAA
>gi|99101122|gb|NP_064634.1|ADAMTS9 1072 aa linear a
disintegrin and metalloproteinase with thrombospondin
motifs-9 preproprotein [Homo sapiens].
MQFVSWATLLTLLVRDLAEMGSPDAAAAVRKDRLHPRQVKLLETLGEYEIVSPIRVNALG
EPFPTNVHFKRTRRSINSATDPWPAFASSSSSSTSSQAHYRLSAFGQQFLFNLTANAGFI
APLFTVTLLGTPGVNQTKFYSEEEAELKHCFYKGYVNTNSEHTAVISLCSGMLGTFRSHD
GDYFIEPLQSMDEQEDEEEQNKPHIIYRRSAPQREPSTGRHACDTSEHKNRHSKDKKKTR
ARKWGERINLAGDVAALNSGLATEAFSAYGNKTDNTREKRTHRRTKRFLSYPRFVEVLVV
ADNRMVSYHGENLQHYILTLMSIVASIYKDPSIGNLINIVIVNLIVIHNEQDGPSISFNA
QTTLKNLCQWQHSKNSPGGIHHDTAVLLTRQDICRAHDKCDTLGLAELGTICDPYRSCSI
SEDSGLSTAFTIAHELGHVFNMPHDDNNKCKEEGVKSPQHVMAPTLNFYTNPWMWSKCSR
KYITEFLDTGYGECLLNEPESRPYPLPVQLPGILYNVNKQCELIFGPGSQVCPYMMQCRR
LWCNNVNGVHKGCRTQHTPWADGTECEPGKHCKYGFCVPKEMDVPVTDGSWGSWSPFGTC
SRTCGGGIKTAIRECNRPEPKNGGKYCVGRRMKFKSCNTEPCLKQKRDFRDEQCAHFDGK
HFNINGLLPNVRWVPKYSGILMKDRCKLFCRVAGNTAYYQLRDRVIDGTPCGQDTNDICV
QGLCRQAGCDHVLNSKARRDKCGVCGGDNSSCKTVAGTFNTVHYGYNTVVRIPAGATNID
VRQHSFSGETDDDNYLALSSSKGEFLLNGNFVVTMAKREIRIGNAVVEYSGSETAVERIN
STDRIEQELLLQVLSVGKLYNPDVRYSFNIPIEDKPQQFYWNSHGPWQACSKPCQGERKR
KLVCTRESDQLTVSDQRCDRLPQPGHITEPCGTDCDLRWHVASRSECSAQCGLGYRTLDI
YCAKYSRLDGKTEKVDDGFCSSHPKPSNREKCSGECNTGGWRYSAWTECSKSCDGGTQRR
RAICVNTRNDVLDDSKCTHQEKVTIQRCSEFPCPQWKSGDWSEVRWEGCYFP
>gi|17981697|gb|NM_001262.2|CDKN2C 2104 bp mRNA Homo sapiens
cyclin-dependent kinase inhibitor 2C (p18, inhibits CDK4)
(CDKN2c), transcript variant 1, mRNA.
CTCTGCCGAGCCTCCTTAAAACTCTGCCGTTAAAATGGGGGCGGGTTTTTCAACTCAAAA
AGCGCTCAATTTTTTTCTTTTCAAAAAAAGCTGATGAGGTCGGAAAAAAGGGAGAAGAAA
CCGGCACCCTCTCTGAGAGGCAACAGAAGCAGCAATTGTTTCAGCGAAAAAAGCAGCAAG
GGAGGGAGTGAAGGAAAAAAGCAAAAAAGGGGGCGACACGCAAGTGCCTGTAGGGGTGAA
AGGAGCAGGGACCGGCGATCTAGGGGGGGATCAGCTACAAAAGAAACTGTCACTGGGAGC
GGTGCGGCCAAGGAGGAAGCAGTGCTGCCAGGCTCTGCTCCAGGGCACAGCTGGCTGGCG
GCTGCCCTGTCCGCAGCAAAGGGGCACAGGCCGGGGACCGCGAGAGGTGGCAAAGTGGCA
CCGGGCGCCGAGGCTGCTGAGCGCTCGCCGAGACGGCGACCGGACTGGCTGCCCCGGAAC
TGCGGCGACTCTCCCTACTCAGAACTTGGCCTACGTTTCCCAGGACTCTCCCCATCTCCA
GAGGCCCCCACAAAACCGGGAAAGGAAGGAAAGGACAGCGGCGGCAGCAGCTCAATGAGT
GCCTACAGCAGAAAGCCTGAACGAGCTCGGTCGTAGGCGGGAAGTTCCCGGGGGGGCTGC
CCAGTGCAGCCGCAATGCTGCCGCGAGCTGCCCCAGCAGTCCGGGCTCCGTAGACGCTTT
CCGCATCACTCTCCTTCCTCGGGCTGCCGGGAGTCCCGGGACCTGGCGGGGCCGGCATGA
CGGGCTTCTCGGGGGCCCGCCGCACGCCCGGCAGCCTCCGGAGACGCGCGCCGAGCCCGG
CTCCCACGGCCTCTGAGGCTCGGCGGGGCTGCGGCTGCCTGGCGGGCGGGCTCCGGAGCT
TTCCTGAGCGGCATTAGCCCACGGCTTGGCCCGGACGCGACCAAAGGCTCTTCTGGAGAA
GCCCAGAGCACTGGGCAATCGTTACGACCTGTAACTTGAGGGCCACCGAACTGCTACTCC
CGTTCGCCTTTGGCGATCATCTTTTAACCCTCCGGAGCACGTCAGCATCCAGCCACCGCG
GCGCTCTCCCAGCAGCGGAGGACCCAGGACTATCCCTTCGGCGAGACGGATGGAAACCGA
GCCCCCTGGAGGACCTGCCCCTGCAGTTCTGCCTCACACGGCTCAAGTCACCACCGTGAA
CAAGGGACCCTAAAGAATGGCCGAGCCTTGGGGGAACGAGTTGGCGTCCGCAGCTGCCAG
GGGGGACCTAGAGCAACTTACTAGTTTGTTGCAAAATAATGTAAACGTCAATGCACAAAA
TGGATTTGGAAGGACTGCGCTGCAGGTTATGAAACTTGGAAATCCCGAGATTGCCAGGAG
ACTGCTACTTAGAGGTGCTAATCCCGATTTGAAAGACCGAACTGGTTTCGCTGTCATTCA
TGATGCGGCCAGAGCAGGTTTCCTGGACACTTTACAGACTTTGCTGGAGTTTCAAGCTGA
TGTTAACATCGAGGATAATGAAGGGAACCTGCCCTTGCACTTGGCTGCCAAAGAAGGCCA
CCTCCGGGTGGTGGAGTTCCTGGTGAAGCACACGGCCAGCAATGTGGGGCATCGGAACCA
TAAGGGGGACACCGCCTGTGATTTGGCCAGGCTCTATGGGAGGAATGAGGTTGTTAGCCT
GATGCAGGCAAACGGGGCTGGGGGAGCCACAAATCTTCAATAAACGTGGGGAGGGCTCCC
CCACGTTGCCTCTACTTTATCAATTAACTGAGTAGCTCTCCTGACTTTTAATGTCATTTG
TTAAAATACAGTTCTGTCATATGTTAAGCAGCTAAATTTTCTGAAACTGCATAAGTGAAA
ATCTTACAACAGGCTTATGAATATATTTAAGCAACATCTTTTTAACCTGCAAAATCTGTT
CTAACATGTAATTGCAGATAACTTTGACTTTCTTCTGAATATTTTATCTTTCCTTGGCTT
TTCCCTTGCTTCCCCTTTTGCCAATCTCAACACCCAAGTTGAAGACTTTGTTTTTAAAAT
GGTTTGTCCTGATGCTTTTGTCTAATTAAAACACTTTCAAAACAGGAAAAAAAAAAAAAA
AAAA
>gi|4502751|gb|NP_001253.1|CDKN2C 168 aa linear cyclin-
dependent kinase inhibitor 2C; cyclin-dependent kinase 6
inhibitor p18; cyclin-dependent kinase 4 inhibitor C;
cyclin-dependent inhibitor; CDK6 inhibitor p18 [Homo
sapiens].
MAEPWGNELASAAARGDLEQLTSLLQNNVNVNAQNGFGRTALQVMKLGNPEIARRLLLRG
ANPDLKDRTGFAVIHDAARAGFLDTLQTLLEFQADVNIEDNEGNLPLHLAAKEGHLRVVE
FLVKHTASNVGHRNHKGDTACDLARLYGRNEVVSLMQANGAGGATNLQ
>gi|23510344|gb|NM_002037.3|FYN 2650 bp mRNA Homo sapiens
FYN oncogene related to SRC, FGR, YES (FYN), transcript
variant 1, mRNA.
GCCGCGCTGGTGGCGGCGGCGCGTCGTTGCAGTTGCGCCATCTGTCAGGAGCGGAGCCGG
CGAGGAGGGGGCTGCCGCGGGCGAGGAGGAGGGGTCGCCGCGAGCCGAAGGCCTTCGAGA
CCCGCCCGCCGCCCGGCGGCGAGAGTAGAGGCGAGGTTGTTGTGCGAGCGGCGCGTCCTC
TCCCGCCCGGGCGCGCCGCGCTTCTCCCAGCGCACCGAGGACCGCCCGGGCGCACACAAA
GCCGCCGCCCGCGCCGCACCGCCCGGCGGCCGCCGCCCGCGCCAGGGAGGGATTCGGCCG
CCGGGCCGGGGACACCCCGGCGCCGCCCCCTCGGTGCTCTCGGAAGGCCCACCGGCTCCC
GGGCCCGCCGGGGACCCCCCGGAGCCGCCTCGGCCGCGCCGGAGGAGGGCGGGGAGAGGA
CCATGTGAGTGGGCTCCGGAGCCTCAGCGCCGCGCAGTTTTTTTGAAGAAGCAGGATGCT
GATCTAAACGTGGAAAAAGACCAGTCCTGCCTCTGTTGTAGAAGACATGTGGTGTATATA
AAGTTTGTGATCGTTGGCGGACATTTTGGAATTTAGATAATGGGCTGTGTGCAATGTAAG
GATAAAGAAGCAACAAAACTGACGGAGGAGAGGGACGGCAGCCTGAACCAGAGCTCTGGG
TACCGCTATGGCACAGACCCCACCCCTCAGCACTACCCCAGCTTCGGTGTGACCTCCATC
CCCAACTACAACAACTTCCACGCAGCCGGGGGCCAAGGACTCACCGTCTTTGGAGGTGTG
AACTCTTCGTCTCATACGGGGACCTTGCGTACGAGAGGAGGAACAGGAGTGACACTCTTT
GTGGCCCTTTATGACTATGAAGCACGGACAGAAGATGACCTGAGTTTTCACAAAGGAGAA
AAATTTCAAATATTGAACAGCTCGGAAGGAGATTGGTGGGAAGCCCGCTCCTTGACAACT
GGAGAGACAGGTTACATTCCCAGCAATTATGTGGCTCCAGTTGACTCTATCCAGGCAGAA
GAGTGGTACTTTGGAAAACTTGGCCGAAAAGATGCTGAGCGACAGCTATTGTCCTTTGGA
AACCCAAGAGGTACCTTTCTTATCCGCGAGAGTGAAACCACCAAAGGTGCCTATTCACTT
TCTATCCGTGATTGGGATGATATGAAAGGAGACCATGTCAAACATTATAAAATTCGCAAA
CTTGACAATGGTGGATACTACATTACCACCCGGGCCCAGTTTGAAACACTTCAGCAGCTT
GTACAACATTACTCAGAGAGAGCTGCAGGTCTCTGCTGCCGCCTAGTAGTTCCCTGTCAC
AAAGGGATGCCAAGGCTTACCGATCTGTCTGTCAAAACCAAAGATGTCTGGGAAATCCCT
CGAGAATCCCTGCAGTTGATCAAGAGACTGGGAAATGGGCAGTTTGGGGAAGTATGGATG
GGTACCTGGAATGGAAACACAAAAGTAGCCATAAAGACTCTTAAACCAGGCACAATGTCC
CCCGAATCATTCCTTGAGGAAGCGCAGATCATGAAGAAGCTGAAGCACGACAAGCTGGTC
CAGCTCTATGCAGTGGTGTCTGAGGAGCCCATCTACATCGTCACCGAGTATATGAACAAA
GGAAGTTTACTGGATTTCTTAAAAGATGGAGAAGGAAGAGCTCTGAAATTACCAAATCTT
GTGGACATGGCAGCACAGGTGGCTGCAGGAATGGCTTACATCGAGCGCATGAATTATATC
CATAGAGATCTGCGATCAGCAAACATTCTAGTGGGGAATGGACTCATATGCAAGATTGCT
GACTTCGGATTGGCCCGATTGATAGAAGACAATGAGTACACAGCAAGACAAGGTGCAAAG
TTCCCCATCAAGTGGACGGCCCCCGAGGCAGCCCTGTACGGGAGGTTCACAATCAAGTCT
GACGTGTGGTCTTTTGGAATCTTACTCACAGAGCTGGTCACCAAAGGAAGAGTGCCATAC
CCAGGCATGAACAACCGGGAGGTGCTGGAGCAGGTGGAGCGAGGCTACAGGATGCCCTGC
CCGCAGGACTGCCCCATCTCTCTGCATGAGCTCATGATCCACTGCTGGAAAAAGGACCCT
GAAGAACGCCCCACTTTTGAGTACTTGCAGAGCTTCCTGGAAGACTACTTTACCGCGACA
GAGCCCCAGTACCAACCTGGTGAAAACCTGTAAGGCCCGGGTCTGCGGAGAGAGGCCTTG
TCCCAGAGGCTGCCCCACCCCTCCCCATTAGCTTTCAATTCCGTAGCCAGCTGCTCCCCA
GCAGCGGAACCGCCCAGGATCAGATTGCATGTGACTCTGAAGCTGACGAACTTCCATGGC
CCTCATTAATGACACTTGTCCCCAAATCCGAACCTCCTCTGTGAAGCATTCGAGACAGAA
CCTTGTTATTTCTCAGACTTTGGAAAATGCATTGTATCGATGTTATGTAAAAGGCCAAAC
CTCTGTTCAGTGTAAATAGTTACTCCAGTGCCAACAATCCTAGTGCTTTCCTTTTTTAAA
AATGCAAATCCTATGTGATTTTAACTCTGTCTTCACCTGATTCAACTAAAAAAAAAAAAG
TATTATTTTCCAAAAGTGGCCTCTTTGTCTAAAACAATAAAATTTTTTTTCATGTTTTAA
CAAAAACCAA
>gi|4503823|gb|NP_002028.1|FYN 537 aa linear protein-
tyrosine kinase fyn isoform a; proto-oncogene tyrosine-
protein kinase fyn; src/yes-related novel gene; src-like
kinase; c-syn protooncogene; tyrosine kinase p59fyn(T);
OKT3-induced calcium influx regulator [Homo sapiens].
MGCVQCKDKEATKLTEERDGSLNQSSGYRYGTDPTPQHYPSFGVTSIPNYNNFHAAGGQG
LTVFGGVNSSSHTGTLRTRGGTGVTLFVALYDYEARTEDDLSFHKGEKFQILNSSEGDWW
EARSLTTGETGYIPSNYVAPVDSIQAEEWYFGKLGRKDAERQLLSFGNPRGTFLIRESET
TKGAYSLSIRDWDDMKGDHVKHYKIRKLDNGGYYITTRAQFETLQQLVQHYSERAAGLCC
RLVVPCHKGMPRLTDLSVKTKDVWEIPRESLQLIKRLGNGQFGEVWMGTWNGNTKVAIKT
LKPGTMSPESFLEEAQIMKKLKHDKLVQLYAVVSEEPIYIVTEYMNKGSLLDFLKDGEGR
ALKLPNLVDMAAQVAAGMAYIERMNYIHRDLRSANILVGNGLICKIADFGLARLIEDNEY
TARQGAKFPIKWTAPEAALYGRFTIKSDVWSFGILLTELVTKGRVPYPGMNNREVLEQVE
RGYRMPCPQDCPISLHELMIHCWKKDPEERPTFEYLQSFLEDYFTATEPQYQPGENL
>gi|15055546|gb|NM_000800.2|FGF1 2357 bp mRNA Homo sapiens
fibroblast growth factor 1 (acidic) (FGF1), transcript
variant 1, mRNA.
GAGCCGGGCTACTCTGAGAAGAAGACACCAAGTGGATTCTGCTTCCCCTGGGACAGCACT
GAGCGAGTGTGGAGAGAGGTACAGCCCTCGGCCTACAAGCTCTTTAGTCTTGAAAGCGCC
ACAAGCAGCAGCTGCTGAGCCATGGCTGAAGGGGAAATCACCACCTTCACAGCCCTGACC
GAGAAGTTTAATCTGCCTCCAGGGAATTACAAGAAGCCCAAACTCCTCTACTGTAGCAAC
GGGGGCCACTTCCTGAGGATCCTTCCGGATGGCACAGTGGATGGGACAAGGGACAGGAGC
GACCAGCACATTCAGCTGCAGCTCAGTGCGGAAAGCGTGGGGGAGGTGTATATAAAGAGT
ACCGAGACTGGCCAGTACTTGGCCATGGACACCGACGGGCTTTTATACGGCTCACAGACA
CCAAATGAGGAATGTTTGTTCCTGGAAAGGCTGGAGGAGAACCATTACAACACCTATATA
TCCAAGAAGCATGCAGAGAAGAATTGGTTTGTTGGCCTCAAGAAGAATGGGAGCTGCAAA
CGCGGTCCTCGGACTCACTATGGCCAGAAAGCAATCTTGTTTCTCCCCCTGCCAGTCTCT
TCTGATTAAAGAGATCTGTTCTGGGTGTTGACCACTCCAGAGAAGTTTCGAGGGGTCCTC
ACCTGGTTGACCCAAAAATGTTCCCTTGACCATTGGCTGCGCTAACCCCCAGCCCACAGA
GCCTGAATTTGTAAGCAACTTGCTTCTAAATGCCCAGTTCACTTCTTTGCAGAGCCTTTT
ACCCCTGCACAGTTTAGAACAGAGGGACCAAATTGCTTCTAGGAGTCAACTGGCTGGCCA
GTCTGGGTCTGGGTTTGGATCTCCAATTGCCTCTTGCAGGCTGAGTCCCTCCATGCAAAA
GTGGGGCTAAATGAAGTGTGTTAAGGGGTCGGCTAAGTGGGACATTAGTAACTGCACACT
ATTTCCCTCTACTGAGTAAACCCTATCTGTGATTCCCCCAAACATCTGGCATGGCTCCCT
TTTGTCCTTCCTGTGCCCTGCAAATATTAGCAAAGAAGCTTCATGCCAGGTTAGGAAGGC
AGCATTCCATGACCAGAAACAGGGACAAAGAAATCCCCCCTTCAGAACAGAGGCATTTAA
AATGGAAAAGAGAGATTGGATTTTGGTGGGTAACTTAGAAGGATGGCATCTCCATGTAGA
ATAAATGAAGAAAGGGAGGCCCAGCCGCAGGAAGGCAGAATAAATCCTTGGGAGTCATTA
CCACGCCTTGACCTTCCCAAGGTTACTCAGCAGCAGAGAGCCCTGGGTGACTTCAGGTGG
AGAGCACTAGAAGTGGTTTCCTGATAACAAGCAAGGATATCAGAGCTGGGAAATTCATGT
GGATCTGGGGACTGAGTGTGGGAGTGCAGAGAAAGAAAGGGAAACTGGCTGAGGGGATAC
CATAAAAAGAGGATGATTTCAGAAGGAGAAGGAAAAAGAAAGTAATGCCACACATTGTGC
TTGGCCCCTGGTAAGCAGAGGCTTTGGGGTCCTAGCCCAGTGCTTCTCCAACACTGAAGT
GCTTGCAGATCATCTGGGGACCTGGTTTGAATGGAGATTCTGATTCAGTGGGTTGGGGGC
AGAGTTTCTGCAGTTCCATCAGGTCCCCCCCAGGTGCAGGTGCTGACAATACTGCTGCCT
TACCCGCCATACATTAAGGAGCAGGGTCCTGGTCCTAAAGAGTTATTCAAATGAAGGTGG
TTCGACGCCCCGAACCTCACCTGACCTCAACTAACCCTTAAAAATGCACACCTCATGAGT
CTACCTGAGCATTCAGGCAGCACTGACAATAGTTATGCCTGTACTAAGGAGCATGATTTT
AAGAGGCTTTGGCCAATGCCTATAAAATGCCCATTTCGAAGATATACAAAAACATACTTC
AAAAATGTTAAACCCTTACCAACAGCTTTTCCCAGGAGACCATTTGTATTACCATTACTT
GTATAAATACACTTCCTGCTTAAACTTGACCCAGGTGGCTAGCAAATTAGAAACACCATT
CATCTCTAACATATGATACTGATGCCATGTAAAGGCCTTTAATAAGTCATTGAAATTTAC
TGTGAGACTGTATGTTTTAATTGCATTTAAAAATATATAGCTTGAAAGCAGTTAAACTGA
TTAGTATTCAGGCACTGAGAATGATAGTAATAGGATACAATGTATAAGCTACTCACTTAT
CTGATACTTATTTACCTATAAAATGAGATTTTTGTTTTCCACTGTGCTATTACAAATTTT
CTTTTGAAAGTAGGAACTCTTAAGCAATGGTAATTGTGAATAAAAATTGATGAGAGTGTT
AAAAAAAAAAAAAAAAA
>gi|4503697|gb|NP_000791.1|FGF1 155 aa linear fibroblast
growth factor 1 (acidic) isoform 1 precursor; heparin-
binding growth factor 1 precursor; endothelial cell growth
factor, alpha; endothelial cell growth factor, beta [Homo
sapiens].
MAEGEITTFTALTEKFNLPPGNYKKPKLLYCSNGGHFLRILPDGTVDGTRDRSDQHIQLQ
LSAESVGEVYIKSTETGQYLAMDTDGLLYGSQTPNEECLFLERLEENHYNTYISKKHAEK
NWFVGLKKNGSCKRGPRTHYGQKAILFLPLPVSSD
>gi|27552761|gb|NM_002825.3|PTN 1029 bp mRNA Homo sapiens
pleiotrophin (heparin binding growth factor 8, neurite
growth-promoting factor 1) (PTN), mRNA.
TCTGCTTTTAATAAGCTTCCCAATCAGCTCTCGAGTGCAAAGCGCTCTCCCTCCCTCGCC
CAGCCTTCGTCCTCCTGGCCCGCTCCTCTCATCCCTCCCATTCTCCATTTCCCTTCCGTT
CCCTCCCTGTCAGGGCGTAATTGAGTCAAAGGCAGGATCAGGTTCCCCGCCTTCCAGTCC
AAAAATCCCGCCAAGAGAGCCCCAGAGCAGAGGAAAATCCAAAGTGGAGAGAGGGGAAGA
AAGAGACCAGTGAGTCATCCGTCCAGAAGGCGGGGAGAGCAGCAGCGGCCCAAGCAGGAG
CTGCAGCGAGCCGGGTACCTGGACTCAGCGGTAGCAACCTCGCCCCTTGCAACAAAGGCA
GACTGAGCGCCAGAGAGGACGTTTCCAACTCAAAAATGCAGGCTCAACAGTACCAGCAGC
AGCGTCGAAAATTTGCAGCTGCCTTCTTGGCATTCATTTTCATACTGGCAGCTGTGGATA
CTGCTGAAGCAGGGAAGAAAGAGAAACCAGAAAAAAAAGTGAAGAAGTCTGACTGTGGAG
AATGGCAGTGGAGTGTGTGTGTGCCCACCAGTGGAGACTGTGGGCTGGGCACACGGGAGG
GCACTCGGACTGGAGCTGAGTGCAAGCAAACCATGAAGACCCAGAGATGTAAGATCCCCT
GCAACTGGAAGAAGCAATTTGGCGCGGAGTGCAAATACCAGTTCCAGGCCTGGGGAGAAT
GTGACCTGAACACAGCCCTGAAGACCAGAACTGGAAGTCTGAAGCGAGCCCTGCACAATG
CCGAATGCCAGAAGACTGTCACCATCTCCAAGCCCTGTGGCAAACTGACCAAGCCCAAAC
CTCAAGCAGAATCTAAGAAGAAGAAAAAGGAAGGCAAGAAACAGGAGAAGATGCTGGATT
AAAAGATGTCACCTGTGGAACATAAAAAGGACATCAGCAAACAGGATCAGTTAACTATTG
CATTTATATGTACCGTAGGCTTTGTATTCAAAAATTATCTATAGCTAAGTACACAATAAG
CAAAAACAA
>gi|4506281|gb|NP_002816.1|PTN 168 aa linear pleiotrophin
(heparin binding growth factor 8, neurite growth-promoting
factor 1); heparin affin regulatory protein; heparin-binding
growth-associated molecule [Homo sapiens].
MQAQQYQQQRRKFAAAFLAFIFILAAVDTAEAGKKEKPEKKVKKSDCGEWQWSVCVPTSG
DCGLGTREGTRTGAECKQTMKTQRCKIPCNWKKQFGAECKYQFQAWGECDLNTALKTRTG
SLKRALHNAECQKTVTISKPCGKLTKPKPQAESKKKKKEGKKQEKMLD
>gi|4504008|gb|NM_000169.1|GLA 1350 bp mRNA Homo sapiens
galactosidase, alpha (GLA), mRNA.
AGGTTAATCTTAAAAGCCCAGGTTACCCGCGGAAATTTATGCTGTCCGGTCACCGTGACA
ATGCAGCTGAGGAACCCAGAACTACATCTGGGCTGCGCGCTTGCGCTTCGCTTCCTGGCC
CTCGTTTCCTGGGACATCCCTGGGGCTAGAGCACTGGACAATGGATTGGCAAGGACGCCT
ACCATGGGCTGGCTGCACTGGGAGCGCTTCATGTGCAACCTTGACTGCCAGGAAGAGCCA
GATTCCTGCATCAGTGAGAAGCTCTTCATGGAGATGGCAGAGCTCATGGTCTCAGAAGGC
TGGAAGGATGCAGGTTATGAGTACCTCTGCATTGATGACTGTTGGATGGCTCCCCAAAGA
GATTCAGAAGGCAGACTTCAGGCAGACCCTCAGCGCTTTCCTCATGGGATTCGCCAGCTA
GCTAATTATGTTCACAGCAAAGGACTGAAGCTAGGGATTTATGCAGATGTTGGAAATAAA
ACCTGCGCAGGCTTCCCTGGGAGTTTTGGATACTACGACATTGATGCCCAGACCTTTGCT
GACTGGGGAGTAGATCTGCTAAAATTTGATGGTTGTTACTGTGACAGTTTGGAAAATTTG
GCAGATGGTTATAAGCACATGTCCTTGGCCCTGAATAGGACTGGCAGAAGCATTGTGTAC
TCCTGTGAGTGGCCTCTTTATATGTGGCCCTTTCAAAAGCCCAATTATACAGAAATCCGA
CAGTACTGCAATCACTGGCGAAATTTTGCTGACATTGATGATTCCTGGAAAAGTATAAAG
AGTATCTTGGACTGGACATCTTTTAACCAGGAGAGAATTGTTGATGTTGCTGGACCAGGG
GGTTGGAATGACCCAGATATGTTAGTGATTGGCAACTTTGGCCTCAGCTGGAATCAGCAA
GTAACTCAGATGGCCCTCTGGGCTATCATGGCTGCTCCTTTATTCATGTCTAATGACCTC
CGACACATCAGCCCTCAAGCCAAAGCTCTCCTTCAGGATAAGGACGTAATTGCCATCAAT
CAGGACCCCTTGGGCAAGCAAGGGTACCAGCTTAGACAGGGAGACAACTTTGAAGTGTGG
GAACGACCTCTCTCAGGCTTAGCCTGGGCTGTAGCTATGATAAACCGGCAGGAGATTGGT
GGACCTCGCTCTTATACCATCGCAGTTGCTTCCCTGGGTAAAGGAGTGGCCTGTAATCCT
GCCTGCTTCATCACACAGCTCCTCCCTGTGAAAAGGAAGCTAGGGTTCTATGAATGGACT
TCAAGGTTAAGAAGTCACATAAATCCCACAGGCACTGTTTTGCTTCAGCTAGAAAATACA
ATGCAGATGTCATTAAAAGACTTACTTTAA
>gi|4504009|gb|NP_000160.1|GLA 429 aa linear galactosidase,
alpha [Homo sapiens].
MQLRNPELHLGCALALRFLALVSWDIPGARALDNGLARTPTMGWLHWERFMCNLDCQEEP
DSCISEKLFMEMAELMVSEGWKDAGYEYLCIDDCWMAPQRDSEGRLQADPQRFPHGIRQL
ANYVHSKGLKLGIYADVGNKTCAGFPGSFGYYDIDAQTFADWGVDLLKFDGCYCDSLENL
ADGYKHMSLALNRTGRSIVYSCEQPLYMWPFQKPNYTEIRQYCNHWRNFADIDDSWKSIK
SILDWTSFNQERIVDVAGPGGWNDPDMLVIGNFGLSWNQQVTQMALWAIMAAPLFMSNDL
RHISPQAKALLQDKDVIAINQDPLGKQGYQLRQGDNFEVWERPLSGLAWAVAMINRQEIG
GPRSYTIAVASLGKGVACNPACFITQLLPVKRKLGFYEWTSRLRSHINPTGTVLLQLENT
MQMSLKDLL
>gi|18587778|gb|XM_091624.1|LOC162542 287 bp mRNA Homo
sapiens similar to ADP-ribosylation factor 1 (LOC162542),
mRNA.
GTCTGATTTTTATGGTTGACAGTAATGACAGAGAGCAGATTGATGAGGCCTGGGAAGTGC
TAACTTACTTGTTAGAGGACGATGAGCTCAGAAATGCAGTTTTATTGGTATTTGCCAATA
AACAAGATCTCCCTAATACTATGAACGCGGCAGAGATAACGGACAAGCTCGGCCTCCATT
CCCTCCGCTACAGAAACTGGCACATTCAGGCTACTTGTGCCACTACTGGACATGGGCTTT
ACGAAGGCCTGAACTGGCTCGCCAACCAGTTCCAGAACCAGAACTGA
>gi|18587779|gb|XP_091624.1|LOC162542 91 aa linear similar
to ADP-ribosylation factor 1 [Homo sapiens].
MVDSNDREQIDEAWEVLTYLLEDDELRNAVLLVFANKQDLPNTMNAAEITDKLGLHSLRY
RNWHIQATCATTGHGLYEGLNWLANQFQNQN
>gi|4557572|gb|NM_000401.1|EXT2 3781 bp mRNA Homo sapiens
exostoses (multiple) 2 (EXT2), mRNA.
CTGTCTGAGCATTTCACTGCGGAGCCTGAGCGCGCCTGCCTGGGAAAACACTGCAGCGGT
GCTCGGACTCCTCCTGTCCAGCAGGAGGCGCGGCCCGGCAGCTCCCGCATGCGCAGTGCG
CTCGGTGTCAGACGGCCCGGATCCCGGTTACCGGCCCCTCGCTCGCTGCTCGCCAGCCCA
GACTCGGCCCTGGCAGTGGCGGCTGGCGATTCGGACCGATCCGACCTGGGCGGAGGTGGC
CCGCGCCCCGCGGCATGAGCCGGTGACCAAGCTCGGGGCCGAGCGGGAGGCAGCCGTGGC
CGAGCCACAGGGATCTGATTCCTCCCAGGGGGATGTCCTGCGCCTCAGGGTCCGGTGGTG
GCCTGCGGCATCCCTTGCGGTGCCAGAAGCCGTGGGACGAGTGTCTTTAATGTTATAGAG
CTACTCAGAGTTGCTGTTTCTCCTTGAGATGCTTTTGGAGTGTGAGGAAGAGGCTGTCTG
TGTCATTATGTGTGCGTCGGTCAAGTATAATATCCGGGGTCCTGCCCTCATCCCAAGAAT
GAAGACCAAGCACCGAATCTACTATATCACCCTCTTCTCCATTGTCCTCCTGGGCCTCAT
TGCCACTGGCATGTTTCAGTTTTGGCCCCATTCTATCGAGTCCTCAAATGACTGGAATGT
AGAGAAGCGCAGCATCCGTGATGTGCCGGTTGTTAGGCTGCCAGCCGACAGTCCCATCCC
AGAGCGGGGGGATCTCAGTTGCAGAATGCACACGTGTTTTGATGTCTATCGCTGTGGCTT
CAACCCAAAGAACAAAATCAAGGTGTATATCTATGCTCTGAAAAAGTACGTGGATGACTT
TGGCGTCTCTGTCAGCAACACCATCTCCCGGGAGTATAATGAACTGCTCATGGCCATCTC
AGACAGTGACTACTACACTGATGACATCAACCGGGCCTGTCTGTTTGTTCCCTCCATCGA
TGTGCTTAACCAGAACACACTGCGCATCAAGGAGACAGCACAAGCGATGGCCCAGCTCTC
TAGGTGGGATCGAGGTACGAATCACCTGTTGTTCAACATGTTGCCTGGAGGTCCCCCAGA
TTATAACACAGCCCTGGATGTCCCCAGAGACAGGGCCCTGTTGGCTGGTGGCGGCTTTTC
TACGTGGACTTACCGGCAAGGCTACGATGTCAGCATTCCTGTCTATAGTCCACTGTCAGC
TGAGGTGGATCTTCCAGAGAAAGGACCAGGTCCACGGCAATACTTCCTCCTGTCATCTCA
GGTGGGTCTCCATCCTGAGTACAGAGAGGACCTAGAAGCCCTCCAGGTCAAACATGGAGA
GTCAGTGTTAGTACTCGATAAATGCACCAACCTCTCAGAGGGTGTCCTTTCTGTCCGTAA
GCGCTGCCACAAGCACCAGGTCTTCGATTACCCACAGGTGCTACAGGAGGCTACTTTCTG
TGTGGTTCTTCGTGGAGCTCGGCTGGGCCAGGCAGTATTGAGCGATGTGTTACAAGCTGG
CTGTGTCCCGGTTGTCATTGCAGACTCCTATATTTTGCCTTTCTCTGAAGTTCTTGACTG
GAAGAGAGCATCTGTGGTTGTACCAGAAGAAAAGATGTCAGATGTGTACAGTATTTTGCA
GAGCATCCCCCAAAGACAGATTGAAGAAATGCAGAGACAGGCCCGGTGGTTCTGGGAAGC
GTACTTCCAGTCAATTAAAGCCATTGCCCTGGCCACCCTGCAGATTATCAATGACCGGAT
CTATCCATATGCTGCCATCTCCTATGAAGAATGGAATGACCCTCCTGCTGTGAAGTGGGG
CAGCGTGAGCAATCCACTCTTCCTCCCGCTGATCCCACCACAGTCTCAAGGGTTCACCGC
CATAGTCCTCACCTACGACCGAGTAGAGAGCCTCTTCCGGGTCATCACTGAAGTGTCCAA
GGTGCCCAGTCTATCCAAACTACTTGTCGTCTGGAATAATCAGAATAAAAACCCTCCAGA
AGATTCTCTCTGGCCCAAAATCCGGGTTCCATTAAAAGTTGTGAGGACTGCTGAAAACAA
GTTAAGTAACCGTTTCTTCCCTTATGATGAAATCGAGACAGAAGCTGTTCTGGCCATTGA
TGATGATATCATTATGCTGACCTCTGACGAGCTGCAATTTGGTTATGAGGTCTGGCGGGA
ATTTCCTGACCGGTTGGTGGGTTACCCGGGTCGTCTGCATCTCTGGGACCATGAGATGAA
TAAGTGGAAGTATGAGTCTGAGTGGACGAATGAAGTGTCCATGGTGCTCACTGGGGCAGC
TTTTTATCACAAGTATTTTAATTACCTGTATACCTACAAAATGCCTGGGGATATCAAGAA
CTGGGTAGATGCTCATATGAACTGTGAAGATATTGCCATGAACTTCCTGGTGGCCAACGT
CACGGGAAAAGCAGTTATCAAGGTAACCCCACGAAAGAAATTCAAGTGTCCTGAGTGCAC
AGCCATAGATGGGCTTTCACTAGACCAAACACACATGGTGGAGAGGTCAGAGTGCATCAA
CAAGTTTGCTTCAGTCTTCGGGACCATGCCTCTCAAGGTGGTGGAACACCGAGCTGACCC
TGTCCTGTACAAAGATGACTTTCCTGAGAAGCTGAAGAGCTTCCCCAACATTGGCAGCTT
ATGAAACGTGTCATTGGTGGAGGTCTGAATGTGAGGCTGGGACAGAGGGAGAGAACAAGG
CCTCCCAGCACTCTGATGTCAGAGTAGTAGGTTAAGGGTGGAAGGTTGACCTACTTGGAT
CTTGGCATGCACCCACCTAACCCACTTTCTCAAGAACAAGAACCTAGAATGAATATCCAA
GCACCTCGAGCTATGCAACCTCTGTTCTTGTATTTCTTATGATCTCTGATGGGTTCTTCT
CGAAAATGCCAAGTGGAAGACTTTGTGGCATGCTCCAGATTTAAATCCAGCTGAGGCTCC
CTTTGTTTTCAGTTCCATGTAACAATCTGGAAGGAAACTTCACGGACAGGAAGACTGCTG
GAGAAGAGAAGCGTGTTAGCCCATTTGAGGTCTGGGGAATCATGTAAAGGGTACCCAGAC
CTCACTTTTAGTTATTTACATCAATGAGTTCTTTCAGGGAACCAAACCCAGAATTCGGTG
CAAAAGCCAAACATCTTGGTGGGATTTGATAAATGCCTTGGGACCTGGAGTGCTGGGCTT
GTGCACAGGAAGAGCACCAGCCGCTGAGTCAGGATCCTGTCAGTTCCATGAGCTATTCCT
CTTTGGTTTGGCTTTTTGATATGATTAAAATTATTTTTTATTCCTTTTTCTACTGTGTCT
TAAACACCAATTCCTGATAGTCCAAGGAACCACCTTTCTCCCTTGATATATTTAACTCCG
TCTTTGGCCTGACAACAGTCTTCTGCCCATGTCTGGGAACACACGCCAGGAGGAATGTCT
GATACCCTCTGCATCAAGCGTAAGAAGGTCCCAAATCATAACCATTTTAAGAACAGATGA
CTCAGAAACCTCCAGAGGAATCTGTTTGCTTCCTGATTAGATCCAGTCAATGTTTTAAAG
GTATTGTCAGAGAAAAACAGAGGGTCTGTACTAGCCATGCAAGGAGTCGCTCTAGCTGGT
ACCCGTAAAAGTTGTGGGATTGTGACCCCCCATCCCAAGGGGATGCCAAAATTTCTCTCA
TTCTTTTGGTATAAACTTAACATTAGCCAGGGAGGTTCTGGCTAACGTTAAATGCTGCTA
TACAACTGCTTTGCAACAGTTGCTGGTATATTTAAATCATTAAATTTCAGCATTTACTAA
T
>gi|4557573|gb|NP_000392.1|EXT2 718 aa linear exostoses
(multiple) 2 [Homo sapiens].
MCASVKYNIRGPALIPRMKTKHRIYYITLFSIVLLGLIATGMFQFWPHSIESSNDWNVEK
RSIRDVPVVRLPADSPIPERGDLSCRMHTCFDVYRCGFNPKNKIKVYIYALKKYVDDFGV
SVSNTISREYNELLMAISDSDYYTDDINRACLFVPSIDVLNQNTLRIKETAQAMAQLSRW
DRGTNHLLFNMLPGGPPDYNTALDVPRDRALLAGGGFSTWTYRQGYDVSIPVYSPLSAEV
DLPEKGPGPRQYFLLSSQVGLHPEYREDLEALQVKHGESVLVLDKCTNLSEGVLSVRKRC
HKHQVFDYPQVLQEATFCVVLRGARLGQAVLSDVLQAGCVPVVIADSYILPFSEVLDWKR
ASVVVPEEKMSDVYSILQSIPQRQIEEMQRQARWFWEAYFQSIKAIALATLQIINDRIYP
YAAISYEEWNDPPAVKWGSVSNPLFLPLIPPQSQGFTAIVLTYDRVESLFRVITEVSKVP
SLSKLLVVWNNQNKNPPEDSLWPKIRVPLKVVRTAENKLSNRFFPYDEIETEAVLAIDDD
IIMLTSDELQFGYEVWREFPDRLVGYPGRLHLWDHEMNKWKYESEWTNEVSMVLTGAAFY
HKYFNYLYTYKMPGDIKNWVDAHMNCEDIANNFLVANVTGKAVIKVTPRKKFKCPECTAI
DGLSLDQTHMVERSECINKFASVFGTMPLKVVEHRADPVLYKDDFPEKLKSFPNIGSL
>gi|27597083|gb|NM_006838.2|METAP2 1908 bp mRNA Homo sapiens
methionyl aminopeptidase 2 (METAP2), mRNA.
CTCTGTCTCATTCCCTCGCGCTCTCTCGGGCAACATGGCGGGTGTGGAGGAGGTAGCGGC
CTCCGGGAGCCACCTGAATGGCGACCTGGATCCAGACGACAGGGAAGAAGGAGCTGCCTC
TACGGCTGAGGAAGCAGCCAAGAAAAAAAGACGAAAGAAGAAGAAGAGCAAAGGGCCTTC
TGCAGCAGGGGAACAGGAACCTGATAAAGAATCAGGAGCCTCAGTGGATGAAGTAGCAAG
ACAGTTGGAAAGATCAGCATTGGAAGATAAAGAAAGAGATGAAGATGATGAAGATGGAGA
TGGCGATGGAGATGGAGCAACTGGAAAGAAGAAGAAAAAGAAGAAGAAGAAGAGAGGACC
AAAAGTTCAAACAGACCCTCCCTCAGTTCCAATATGTGACCTGTATCCTAATGGTGTATT
TCCCAAAGGACAAGAATGCGAATACCCACCCACACAAGATGGGCGAACAGCTGCTTGGAG
AACTACAAGTGAAGAAAAGAAAGCATTAGATCAGGCAAGTGAAGAGATTTGGAATGATTT
TCGAGAAGCTGCAGAAGCACATCGACAAGTTAGAAAATACGTAATGAGCTGGATCAAGCC
TGGGATGACAATGATAGAAATCTGTGAAAAGTTGGAAGACTGTTCACGCAAGTTAATAAA
AGAGAATGGATTAAATGCAGGCCTGGCATTTCCTACTGGATGTTCTCTCAATAATTGTGC
TGCCCATTATACTCCCAATGCCGGTGACACAACAGTATTACAGTATGATGACATCTGTAA
AATAGACTTTGGAACACATATAAGTGGTAGGATTATTGACTGTGCTTTTACTGTCACTTT
TAATCCCAAATATGATACGTTATTAAAAGCTGTAAAAGATGCTACTAACACTGGAATAAA
GTGTGCTGGAATTGATGTTCGTCTGTGTGATGTTGGTGAGGCCATCCAAGAAGTTATGGA
GTCCTATGAAGTTGAAATAGATGGGAAGACATATCAAGTGAAACCAATCCGTAATCTAAA
TGGACATTCAATTGGGCAATATAGAATACATGCTGGAAAAACAGTGCCGATTGTGAAAGG
AGGGGAGGCAACAAGAATGGAGGAAGGAGAAGTATATGCAATTGAAACCTTTGGTAGTAC
AGGAAAAGGTGTTGTTCATGATGATATGGAATGTTCACATTACATGAAAAATTTTGATGT
TGGACATGTGCCAATAAGGCTTCCAAGAACAAAACACTTGTTAAATGTCATCAATGAAAA
CTTTGGAACCCTTGCCTTCTGCCGCAGATGGCTGGATCGCTTGGGAGAAAGTAAATACTT
GATGGCTCTGAAGAATCTGTGTGACTTGGGCATTGTAGATCCATATCCACCATTATGTGA
CATTAAAGGATCATATACAGCGCAATTTGAACATACCATCCTGTTGCGTCCAACATGTAA
AGAAGTTGTCAGCAGAGGAGATGACTATTAAACTTAGTCCAAAGCCACCTCAACACCTTT
ATTTTCTGAGCTTTGTTGGAAAACATGATACCAGAATTAATTTGCCACATGTTGTCTGTT
TTAACAGTGGACCCATGTAATACTTTTATCCATGTTTAAAAAGAAGGAATTTGGACAAAG
GCAAACCGTCTAATGTAATTAACCAACGAAAAAGCTTTCCGGACTTTTAAATGCTAACTG
TTTTTCCCCTTCCTGTCTAGGAAAATGCTATAAAGCTCAAATTAGTTAGGAATGACTTAT
ACGTTTTGTTTTGAATACCTAAGAGATACTTTTTGGATATTTATATTGCCATATTCTTAC
TTGAATGCTTTGAATGACTACATCCAGTTCTGCACCTATACCCTCTGGTGTTGCTTTTTA
ACCTTCCTGGAATCCATTTCTAAAAAATAAAGACATTTTCAGATCTGA
>gi|5803092|gb|NP_006829.1|METAP2 478 aa linear methionyl
aminopeptidase 2; methionine aminopeptidase; eIF-2-
associated p67 [Homo sapiens].
MAGVEEVAASGSHLNGDLDPDDREEGAASTAEEAAKKKRRKKKKSKGPSAAGEQEPDKES
GASVDEVARQLERSALEDKERDEDDEDGDGDGDGATGKKKKKKKKKRGPKVQTDPPSVPI
CDLYPNGVFPKGQECEYPPTQDGRTAAWRTTSEEKKALDQASEEIWNDFREAAEAHRQVR
KYVMSWIKPGMTMIEICEKLEDCSRKLIKENGLNAGLAFPTGCSLNNCAAHYTPNAGDTT
VLQYDDICKIDFGTHISGRIIDCAFTVTFNPKYDTLLKAVKDATNTGIKCAGIDVRLCDV
GEAIQEVMESYEVEIDGKTYQVKPIRNLNGHSIGQYRIHAGKTVPIVKGGEATRMEEGEV
YAIETFGSTGKGVVHDDMECSHYMKNFDVGHVPIRLPRTKHLLNVINENFGTLAFCRRWL
DRLGESKYLMALKNLCDLGIVDPYPPLCDIKGSYTAQFEHTILLRPTCKEVVSRGDDY
>gi|10864040|gb|NM_021230.1|MLL3 12689 bp mRNA Homo sapiens
myeloid/lymphoid or mixed-lineage leukemia3 (MLL3), mRNA.
AAAATTCCTTAGTTGCTGGCTTTGACCTTTTATGTTGCTGAGTTTTACACATCTATTTTC
TCAACTGCCATATCCTAGGGGGCTTGGAGTACCCATAATACAGTGAGCCCACCTTCCTGG
TCCCCAGACATTTCAGAAGGTCGGGAAATTTTTAAACCCAGGCAGCTTCCTGGCAGTGCC
ATTTGGAGCATCAAAGTGGGCCGTGGGTCTGGATTTCCAGGAAAGCGGAGACCTCGAGGT
GCAGGACTGTCGGGGCGAGGTGGCCGAGGCAGGTCAAAGCTGAAAAGTGGAATCGGAGCT
GTTGTATTACCTGGGGTGTCTACTGCAGATATTTCATCAAATAAGGATGATGAAGAAAAC
TCTATGCACAATACAGTTGTGTTGTTTTCTAGCAGTGACAAGTTCACTTTGAATCAGGAT
ATGTGTGTAGTTTGTGGCAGTTTTGGCCAAGGAGCAGAAGGAAGATTACTTGCCTGTTCT
CAGTGTGGTCAGTGTTACCATCCATACTGTGTCAGTATTAAGATCACTAAAGTGGTTCTT
AGCAAAGGTTGGAGGTGTCTTGAGTGCACTGTGTGTGAGGCCTGTGGGAAGGCAACTGAC
CCAGGAAGACTCCTGCTGTGTGATGACTGTGACATAAGTTATCACACCTACTGCCTAGAC
CCTCCATTGCAGACAGTTCCCAAAGGAGGCTGGAAGTGCAAATGGTGTGTTTGGTGCAGA
CACTGTGGAGCAACATCTGCAGGTCTAAGATGTGAATGGCAGAACAATTACACACAGTGC
GCTCCTTGTGCAAGCTTATCTTCCTGTCCAGTCTGCTATCGAAACTATAGAGAAGAAGAT
CTTATTCTGCAATGTAGACAATGTGATAGATGGATGCATGCAGTTTGTCAGAACTTAAAT
ACTGAGGAAGAAGTGGAAAATGTAGCAGACATTGGTTTTGATTGTAGCATGTGCAGACCC
TATATGCCTGCGTCTAATGTGCCTTCCTCAGACTGCTGTGAATCTTCACTTGTAGCACAA
ATTGTCACAAAAGTAAAAGAGCTAGACCCACCCAAGACTTATACCCAGGATGGTGTGTGT
TTGACTGAATCAGGGATGACTCAGTTACAGAGCCTCACAGTTACAGTTCCAAGAAGAAAA
CGGTCAAAACCAAAATTGAAATTGAAGATTATAAATCAGAATAGCGTGGCCGTCCTTCAG
ACCCCTCCAGACATCCAATCAGAGCATTCAAGGGATGGTGAAATGGATGATAGTCGAGAA
GGAGAACTTATGGATTGTGATGGAAAATCAGAATCTAGTCCTGAGCGGGAAGCTGTGGAT
GATGAAACTAAGGGAGTGGAAGGAACAGATGGTGTCAAAAAGAGAAAAAGGAAACCATAC
AGACCAGGTATTGGTGGATTTATGGTGCGGCAAAGAAGTCGAACTGGGCAAGGGAAAACC
AAAAGATCTGTGATCAGAAAAGATTCCTCAGGCTCTATTTCCGAGCAGTTACCTTGCAGA
GATGATGGCTGGAGTGAGCAGTTACCAGATACTTTAGTTGATGAATCTGTTTCTGTTACT
GAAAGCACTGAAAAAATAAAGAAGAGATACCGAAAAAGGAAAAATAAGCTTGAAGAAACT
TTCCCTGCCTATTTACAAGAAGCTTTCTTTGGAAAAGATCTTCTAGATACAAGTAGACAA
AGCAAGATAAGTTTAGATAATCTGTCAGAAGATGGAGCTCAGCTTTTATATAAAACAAAC
ATGAACACAGGTTTCTTGGATCCTTCCTTAGATCCACTACTTAGTTCATCCTCGGCTCCA
ACAAAATCTGGAACTCACGGTCCTGCTGATGACCCATTAGCTGATATTTCTGAAGTTTTA
AACACAGATGATGACATTCTTGGAATAATTTCAGATGATCTAGCAAAATCAGTTGATCAT
TCAGATATTGGTCCTGTCACTGATGATCCTTCCTCTTTGCCTCAGCCAAATGTCAATCAG
AGTTCACGACCATTAAGTGAAGAACAGCTAGATGGGATCCTCAGTCCTGAACTAGACAAA
ATGGTCACAGATGGAGCAATTCTTGGAAAATTATATAAAATTCCAGAGCTTGGCGGAAAA
GATGTTGAAGACTTATTTACAGCTGTACTTAGTCCTGCGAACACTCAGCCAACTCCATTG
CCACAGCCTCCCCCACCAACACAGCTGTTGCCAATACACAATCAGGATGCTTTTTCACGG
ATGCCTCTCATGAATGGCCTTATTGGATCCAGTCCTCATCTCCCACATAATTCTTTGCCA
CCTGGAAGCGGACTGGGAACTTTCTCTGCAATTGCACAATCCTCTTATCCTGATGCCAGG
GATAAAAATTCAGCCTTTAATCCAATGGCAAGTGATCCTAACAACTCTTGGACATCATCA
GCTCCCACTGTGGAAGGAGAAAATGACACAATGTCGAATGCCCAGAGAAGCACGCTTAAG
TGGGAGAAAGAGGAGGCTCTGGGTGAAATGGCAACTGTTGCCCCAGTTCTCTACACCAAT
ATTAATTTCCCCAACTTAAAGGAAGAATTCCCTGATTGGACTACTAGAGTGAAGCAAATT
GCCAAATTGTGGAGAAAAGCAAGCTCACAAGAAAGAGCACCATATGTGCAAAAAGCCAGA
GATAACAGAGCTGCTTTACGCATTAATAAAGTACAGATGTCAAATGATTCCATGAAAAGG
CAGCAACAGCAAGATAGCATTGATCCCAGCTCTCGTATTGATTCGGAGCTTTTTAAAGAT
CCTTTAAAGCAAAGAGAATCAGAACATGAACAGGAATGGAAATTTAGACAGCAAATGCGT
CAGAAAAGTAAGCAGCAAGCTAAAATTGAAGCCACACAGAAACTTGAACAGGTGAAAAAT
GAGCAGCAGCAGCAGCAACAACAGCAATTTGGTTCTCAGCATCTTCTGGTGCAGTCTGGT
TCAGATACACCAAGTAGTGGGATACAGAGTCCCTTGACACCTCAGCCTGGCAATGGAAAT
ATGTCTCCTGCACAGTCATTCCATAAAGAACTGTTTACAAAACAGCCACCCAGTACCCCT
ACGTCTACATCTTCAGATGATGTGTTTGTAAAGCCACAAGCTCCACCTCCTCCTCCAGCC
CCATCCCGGATTCCCATCCAGGATAGTCTTTCTCAGGCTCAGACTTCTCAGCCACCCTCA
CCGCAAGTGTTTTCACCTQGGTCCTCTAACTCACGACCACCATCTCCAATGGATCCATAT
GCAAAAATGGTTGGTACCCCTCGACCACCTCCTGTGGGCCATAGTTTTTCCAGAAGAAAT
TCTGCTGCACCAGTGGAAAACTGTACACCTTTATCATCGGTATCTAGGCCCCTTCAAATG
AATGAGACAACAGCAAATAGGCCATCCCCTGTCAGAGATTTATGTTCTTCTTCCACGACA
AATAATGACCCCTATGCAAAACCTCCAGACACACCTAGGCCTGTGATGACAGATCAATTT
CCCAAATCCTTGGGCCTATCCCGGTCTCCTGTAGTTTCAGAACAAACTGCAAAAGGCCCT
ATAGCAGCTGGAACCAGTGATCACTTTACTAAACCATCTCCTAGGGCAGATGTGTTTCAA
AGACAAAGGATACCTGACTCATATGCACGACCCTTGTTGACACCTGCACCTCTTGATAGT
GGTCCTGGACCTTTTAAGACTCCAATGCAACCTCCTCCATCCTCTCAGGATCCTTATGGA
TCAGTGTCACAGGCATCAAGGCGATTGTCTGTTGACCCTTATGAAAGGCCTGCTTTGACA
CCAAGACCTATAGATAATTTTTCTCATAATCAGTCAAATGATCCATATAGTCAGCCTCCC
CTTACCCCACATCCAGCAGTGAATGAATCTTTTGCCCATCCTTCAAGGGCTTTTTCCCAG
CCTGGAACCATATCAAGGCCAACATCTCAGGACCCATACTCCCAACCCCCAGGAACTCCA
CGACCTGTTGTAGATTCTTATTCCCAATCTTCAGGAACAGCTAGGTCCAATACAGACCCT
TACTCTCAACCTCCTGGAACTCCCCGGCCTACTACTGTTGACCCATATAGTCAGCAGCCC
CAAACCCCAAGACCATCTACACAAACTGACTTGTTTGTTACACCTGTAACAAATCAGAGG
CATTCTGATCCATATGCTCATCCTCCTGGAACACCAAGACCTGGAATTTCTGTCCCTTAC
TCTCAGCCACCAGCAACACCAAGGCCAAGGATTTCAGAGGGTTTTACTAGGTCCTCAATG
ACAAGACCAGTCCTCATGCCAAATCAGGATCCTTTCCTGCAAGCAGCACAAAACCGAGGA
CCAGCTTTACCTGGCCCGTTGGTAAGGCCACCTGATACATGTTCCCAGACACCTAGGCCC
CCTGGACCTGGTCTTTCAGACACATTTAGCCGTGTTTCCCCATCTGCTGCCCGTGATCCC
TATGATCAGTCTCCAATGACTCCAAGATCTCAGTCTGACTCTTTTGGAACAAGTCAAACT
GCCCATGATGTTGCTGATCAGCCAAGGCCTGGATCAGAGGGGAGCTTCTGTGCATCTTCA
AACTCTCCAATGCACTCCCAAGGCCAGCAGTTCTCTGGTGTCTCCCAACTTCCTGGACCT
GTGCCAACTTCAGGAGTAACTGATACACAGAATACTGTAAATATGGCCCAAGCAGATACA
GAGAAATTGAGACAGCGGCAGAAGTTACGTGAAATCATTCTCCAGCAGCAACAGCAGAAG
AAGATTGCAGGTCGACAGGAGAAGGGGTCACAGGACTCACCCGCAGTGCCTCATCCAGGG
CCTCTTCAACACTGGCAACCAGAGAATGTTAACCAGGCTTTCACCAGACCCCCACCTCCC
TATCCTGGGAACATTAGGTCTCCTGTTGCCCCTCCTTTAGGACCTAGATATGCTGTTTTC
CCAAAAGATCAGCGTGGACCCTATCCTCCTGATGTTGCTAGTATGGGGATGAGACCTCAT
GGATTTAGATTTGGATTTCCAGGAGGTAGTCATGGTACCATGCCGAGTCAAGAGCGCTTC
CTTGTGCCTCCTCAGCAAATACAGGGATCTGGAGTTTCTCCACAGCTAAGAAGATCAGTA
TCTGTAGATATGCCTAGGCCTTTAAATAACTCACAAATGAATAATCCAGTTGGACTTCCT
CAGCATTTTTCACCACAGAGCTTGCCAGTTCAGCAGCACAACATACTGGGCCAAGCATAT
ATTGAACTGAGACATAGGGCTCCTGACGGAAGGCAACGGCTGCCTTTCAGTGCTCCACCT
GGCAGCGTTGTAGAGGCATCTTCTAATCTGAGACATGGAAACTTCATTCCCCGGCCAGAC
TTTCCGGGCCCTAGACACACAGACCCCATGCGACGACCTCCCCAGGGTCTACCTAATCAG
CTACCTGTGCACCCAGATTTGGAACAAGTGCCACCATCTCAACAAGAGCAAGGTCATTCT
GTCCATTCATCTTCTATGGTCATGAGGACTCTGAACCATCCACTAGGTGGTGAATTTTCA
GAAGCTCCTTTGTCAACATCTGTACCGTCTGAAACAACGTCTGATAATTTACAGATAACC
ACCCAGCCTTCTGATGGTCTAGAGGAAAAACTTGATTCTGATGACCCTTCTGTGAAGGAA
CTGGATGTTAAAGACCTTGAGGGGGTTGAAGTCAAAGACTTAGATGATGAAGATCTTGAA
AACTTAAATTTAGATACAGAGGATGGCAAGGTAGTTGAATTGGATACTTTAGATAATTTG
GAAACTAATGATCCCAACCTGGATGACCTCTTAAGGTCAGGAGAGTTTGATATCATTGCA
TATACAGATCCAGAACTTGACATGGGAGATAAGAAAAGCATGTTTAATGAGGAACTAGAC
CTTCCAATTGATGATAAGTTAGATAATCAGTGTGTATCTGTTGAACCAAAAAAAAAGGAA
CAAGAAAACAAAACTCTGGTTCTCTCTGATAAACATTCACCACAGAAAAAATCCACTGTT
ACCAATGAGGTAAAAACGGAAGTACTGTCTCCAAATTCTAAGGTGGAATCCAAATGTGAA
ACTGAAAAAAATGATGAGAATAAAGATAATGTTGACACTCCTTGCTCACAGGCTTCTGCT
CACTCAGACCTAAATGATGGAGAAAAGACTTCTTTGCATCCTTGTGATCCAGATCTATTT
GAGAAAAGAACCAATCGAGAAACTGCTGGCCCCAGTGCAAATGTCATTCAGGCATCCACT
CAACTACCTGCTCAAGATGTAATAAACTCTTGTGGCATAACTGGATCAACTCCAGTTCTC
TCAAGTTTACTTGCTAATGAGAAATCTGATAATTCAGACATTAGGCCATCGGGGTCTCCA
CCACCACCAACTCTGCCGGCCTCCCCATCCAATCATGTGTCAAGTTTGCCTCCTTTCATA
GCACCGCCTGGCCGTGTTTTGGATAATGCCATGAATTCTAATGTGACAGTAGTCTCTAGG
GTAAACCATGTTTTTTCTCAGGGTGTGCAGGTAAACCCAGGGCTCATTCCAGGTCAATCA
ACAGTTAACCACAGTCTGGGGACAGGAAAACCTGCAACTCAAACTGGGCCTCAAACAAGT
CAGTCTGGTACCAGTAGCATGTCTGGACCCCAACAGCTAATGATTCCTCAAACATTAGCA
CAGCAGAATAGAGAGAGGCCCCTTCTTCTAGAAGAACAGCCTCTACTTCTACAGGATCTT
TTGGATCAAGAAAGGCAAGAACAGCAGCAGCAAAGACAGATGCAAGCCATGATTCGTCAG
CGATCAGAACCGTTCTTCCCTAATATTGATTTTGATGCAATTACAGATCCTATAATGAAA
GCCAAAATGGTGGCCCTTAAAGGTATAAATAAAGTGATGGCACAAACAAATCTGGGCATG
CCACCAATGGTGATGAGCAGGTTCCCTTTTATGGGCCAGGTGGTAACTGGAACACAGAAC
AGTGAAGGACAGAACCTTGGACCACAGGCCATTCCTCAGGATGGCAGTATAACACATCAG
ATTTCTAGGCCTAATCCTCCAAATTTTGGTCCAGGCTTTGTCAATGATTCACAGCGTAAG
CAGTATGAAGAGTGGCTCCAGGAGACCCAACAGCTGCTTCAAATGCAGCAGAAGTATCTT
GAAGAACAAATTGGTGCTCACAGAAAATCTAAGAAGGCCCTTTCAGCTAAACAACGTACT
GCCAAGAAAGCTGGGCGTGAATTTCCAGAGGAAGATGCAGAACAACTCAAGCATGTTACT
GAACAGCAAAGCATGGTTCAGAAACAGCTAGAACAGATTCGTAAACAACAGAAAGAACAT
GCTGAATTGATTGAAGATTATCGGATCAAACAGCAGCAGCAATGTGCAATGGCCCCACCT
ACCATGATGCCCAGTGTCCAGCCCCAGCCACCCCTAATTCCAGGTGCCACTCCACCCACC
ATGAGCCAACCCACCTTTCCCATGGTGCCACAGCAGCTTCAGCACCAGCAGCACACAACA
GTTATTTCTGGCCATACTAGCCCTGTTAGAATGCCCAGTTTACCTGGATGGCAACCCAAC
AGTGCTCCTGCCCACCTGCCCCTCAATCCTCCTAGAATTCAGCCCCCAATTGCCCAGTTA
CCAATAAAAACTTGTACACCAGCCCCAGGGACAGTCTCAAATGCAAATCCACAGAGTGGA
CCACCACCTCGGGTAGAATTTGATGACAACAATCCCTTTAGTGAAAGTTTTCAAGAACGG
GAACGTAAGGAACGTTTACGAGAACAGCAAGAGAGACAACGGATCCAACTCATGCAGGAG
GTAGATAGACAAAGAGCTTTGCAGCAGAGGATGGAAATGGAGCAGCATGGTATGGTGGGC
TCTGAGATAAGTAGTAGTAGGACATCTGTGTCCCAGATTCCCTTCTACAGTTCCGACTTA
CCTTGTGATTTTATGCAACCTCTAGGACCCCTTCAGCAGTCTCCACAACACCAACAGCAA
ATGGGGCAGGTTTTACAGCAGCAGAATATACAACAAGGATCAATTAATTCACCCTCCACC
CAAACTTTCATGCAGACTAATGAGCGAAGGCAGGTAGGCCCTCCTTCATTTGTTCCTGAT
TCACCATCAATCCCTGTTGGAAGCCCAAATTTTTCTTCTGTGAAGCAGGGACATGGAAAT
CTTTCTGGGACCAGCTTCCAGCAGTCCCCAGTGAGGCCTTCTTTTACACCTGCTTTACCA
GCAGCACCTCCAGTAGCTAATAGCAGTCTCCCATGTGGCCAAGATTCTACTATAACCCAT
GGACACAGTTATCCGGGATCAACCCAATCGCTCATTCAGTTGTATTCTGATATAATCCCA
GAGGAAAAAGGGAAAAAGAAAAGAACAAGAAAGAAGAAAAGAGATGATGATGCAGAATCC
ACCAAGGCTCCATCAACTCCCCATTCAGATATAACTGCCCCACCGACTCCAGGCATCTCA
GAAACTACCTCTACTCCTGCAGTGAGCACACCCAGTGAGCTTCCTCAACAAGCCGACCAA
GAGTCGGTGGAACCAGTCGGCCCATCCACTCCCAATATGGCAGCAGGCCAGCTATGTACA
GAATTAGAGAACAAACTGCCCAATAGTGATTTCTCACAAGCAACTCCAAATCAACAGACG
TATGCAAATTCAGAAGTAGACAAGCTCTCCATGGAAACCCCTGCCAAAACAGAAGAGATA
AAACTGGAAAAGGCTGAGACAGAGTCCTGCCCAGGCCAAGAGGAGCCTAAATTGGAGGAA
CAGAATGGTAGTAAGGTAGAAGGAAACGCTGTAGCCTGTCCTGTCTCCTCAGCACAGAGT
CCTCCCCATTCTGCTGGGGCCCCTGCTGCCAAAGGAGACTCAGGGAATGAACTTCTGAAA
CACTTGTTGAAAAATAAAAAGTCATCTTCTCTTTTGAATCAAAAACCTGAGGGCAGTATT
TGTTCAGAAGATGACTGTACAAAGGATAATAAACTAGTTGAGAAGCAGAACCCAGCTGAA
GGACTGCAAACTTTGGGGGCTCAAATGCAAGGTGGTTTTGGATGTGGCAACCAGTTGCCA
AAAACAGATGGAGGAAGTGAAACCAAGAAACAGCGAAGCAAACGGACTCAGAGGACGGGT
GAGAAAGCAGCACCTCGCTCAAAGAAAAGGAAAAAGGACGAAGAGGAGAAACAAGCTATG
TACTCTAGCACTGACACGTTTACCCACTTGAAACAGGTGAGGCAGCTCTCTCTGCTCCCT
CTAATGGAACCAATCATTGGAGTGAACTTTGCGCACTTTCTTCCTTATGGCAGTGGCCAA
TTTAATAGTGGGAATCGACTTCTAGGAACTTTTGGCAGTGCTACCCTGGAAGGGGTTTCG
GACTACTATTCTCAGTTGATCTACAAGCAGAATAATTTAAGTAATCCTCCAACACCCCCT
GCCTCTCTTCCTCCTACACCACCTCCTATGGCTTGTCAGAAGATGGCCAATGGTTTTGCA
ACAACTGAAGAACTTGCTGGAAAAGCCGGAGTGTTAGTGAGCCATGAAGTTACCAAAACT
CTAGGACCTAAACCATTTCAGCTGCCCTTCAGACCCCAGGACGACTTGTTGGCCCGAGCT
CTTGCTCAGGGCCCCAAGACAGTTGATGTGCCAGCCTCCCTCCCAACACCACCTCATAAC
AATCAGGAAGAATTAAGGATACAGGATCACTGTGGTGATCGAGATACTCCTGACAGTTTT
GTTCCCTCATCCTCTCCTGAGAGTGTGGTTGGGGTAGAAGTGAGCAGGTATCCAGATCTG
TCATTGGTCAAGGAGGAGCCTCCAGAACCGGTGCCGTCCCCCATCATTCCAATTCTTCCT
AGCACTGCTGGGAAAAGTTCAGAATCAAGAAGGAATGACATCAAAACTGAGCCAGGCACT
TTATATTTTGCGTCACCTTTTGGTCCTTCCCCAAATGGTCCCAGATCAGGTCTTATATCT
GTAGCAATTACTCTGCATCCTACAGCTGCTGAGAACATTAGCAGTGTTGTGGCTGCATTT
TCCGACCTTCTTCACGTCCGAATCCCTAACAGCTATGAGGTTAGCAGTGCTCCAGATGTC
CCATCCATGGGTTTGGTCAGTAGCCACAGAATCAACCCGGGTTTGGAGTATCGACAGCAT
TTACTTCTCCGTGGGCCTCCGCCAGGATCTGCAAACCCTCCCAGATTAGTGAGCTCTTAC
CGGCTGAAGCAGCCTAATGTACCATTTCCTCCAACAAGCAATGGTCTTTCTGGATATAAG
GATTCTAGTCATGGTATTGCAGAAAGCGCAGCACTCAGACCACAGTGGTGTTGTCATTGT
AAAGTGGTTATTCTTGGAAGTGGTGTGCGGAAATCTTTCAAAGATCTGACCCTTTTGAAC
AAGGATTCCCGAGAAAGCACCAAGAGGGTAGAGAAGGACATTGTCTTCTGTAGTAATAAC
TGCTTTATTCTTTATTCATCAACTGCACAAGCGAAAAACTCAGAAAACAAGGAATCCATT
CCTTCATTGCCACAATCACCTATGAGAGAAACGCCTTCCAAAGCATTTCATCAGTACAGC
AACAACATCTCCACTTTGGATGTGCACTGTCTCCCCCAGCTCCCAGAGAAAGCTTCTCCC
CCTGCCTCACCACCCATCGCCTTCCCTCCTGCTTTTGAAGCAGCCCAAGTCGAGGCCAAG
CCAGATGAGCTGAAGGTGACAGTCAAGCTGAAGCCTCGGCTAAGAGCTGTCCATGGTGGG
TTTGAAGATTGCAGGCCGCTCAATAAAAAATGGAGAGGAATGAAATGGAAGAAGTGGAGC
ATTCATATTGTAATCCCTAAGGGGACATTTAAACCACCTTGTGAGGATGAAATAGATGAA
TTTCTAAAGAAATTGGGCACTTCCCTTAAACCTGATCCTGTGCCCAAAGACTATCGGAAA
TGTTGCTTTTGTCATGAAGAAGGTGATGGATTGACAGATGGACCAGCAAGGCTACTCAAC
CTTGACTTGGATCTGTGGGTCCACTTGAACTGCGCTCTGTGGTCCACGGAGGTCTATGAG
ACTCAGGCTGGTGCCTTAATAAATGTGGAGCTAGCTCTGAGGAGAGGCCTACAAATGAAA
TGTGTCTTCTGTCACAAGACGGGTGCCACTAGTGGATGCCACAGATTTCGATGCACCAAC
ATTTATCACTTCACTTGCGCCATTAAAGCACAATGCATGTTTTTTAAGGACAAAACTATG
CTTTGCCCCATGCACAAACCAAAGGGAATTCATGAGCAAGAATTAAGTTACTTTGCAGTC
TTCAGGAGGGTCTATGTTCAGCGTGATGAGGTGCGACAGATTGCTAGCATCGTGCAACGA
GGAGAACGGGACCATACCTTTCGCGTGGGTAGCCTCATCTTCCACACAATTGGTCAGCTG
CTTCCACAGCAGATGCAAGCATTCCATTCTCCTAAAGCACTCTTCCCTGTGGGCTATGAA
GCCAGCCGGCTGTACTGGAGCACTCGCTATGCCAATAGGCGCTGCCGCTACCTGTGCTCC
ATTGAGGAGAAGGATGGGCGCCCAGTGTTTGTCATCAGGATTGTGGAACAAGGCCATGAA
GACCTGGTTCTAAGTGACATCTCACCTAAAGGTGTCTGGGATAAGATTTTGGAGCCTGTG
GCATGTGTGAGAAAAAAGTCTGAAATGCTCCAGCTTTTCCCAGCGTATTTAAAAGGAGAG
GATCTGTTTGGCCTGACCGTCTCTGCAGTGGCACGCATAGCGGAATCACTTCCTGGGGTT
GAGGCATGTGAAAATTATACCTTCCGATACGGCCGAAATCCTCTCATGGAACTTCCTCTT
GCCGTTAACCCCACAGGTTGTGCCCGTTCTGAACCTAAAATGAGTGCCCATGTCAAGAGG
CCTCACACCTTAAACAGCACCAGCACCTCAAAGTCATTTCAGAGCACAGTCACTGGAGAA
CTGAACGCACCTTATAGTAAACAGTTTGTTCACTCCAAGTCATCGCAGTACCGGAAGATG
AAAACTGAATGGAAATCCAATGTGTATCTGGCACGGTCTCGGATTCAGGGGCTGGGCCTG
TATGCTGCTCGAGACATTGAGAAACACACCATGGTCATTGAGTACATCGGGACTATCATT
CGAAACGAAGTAGCCAACAGGAAAGAGAAGCTTTATGAGTCTCAGAACCGTGGTGTGTAC
ATGTTCCGCATGGATAACGACCATGTGATTGACGCGACGCTCACAGGAGGGCCCGCAAGG
TATATCAACCATTCGTGTGCACCTAATTGTGTGGCTGAAGTGGTGACTTTTGAGAGAGGA
CACAAAATTATCATCAGCTCCAGTCGGAGAATCCAGAAAGGAGAAGAGCTCTGCTATGAC
TATAAGTTTGACTTTGAAGATGACCAGCACAAGATTCCGTGTCACTGTGGAGCTGTGAAC
TGCCGGAAGTGGATGAACTGAAATGCATTCCTTGCTAGCTCAGCGGGCGGCTTGTCCCTA
GGAAGAGGCGATTCAACACACCATTGGAATTTTGCAGACAGAAAGAGATTTTTGTTTTCT
GTTTTATGACTTTTTGAAAAAGCTTCTGGGAGTTCTGATTTCCTCAGTCCTTTAGGTTAA
AGCAGCGCCAGGAGGAAGCTGACAGAAGCAGCGTTCCTGAAGTGGCCGAGGTTAAACGGA
ATCACAGAATGGTCCAGCACTTTTGCTTT
>gi|10864041|gb|NP_067053.1|MLL3 4025 aa linear myeloid/
lynphoid or mixed-lineage leukemia 3; ALR-like protein
[Homo sapiens].
MRNTVVLFSSSDKFTLNQDMCVVCGSFGQGAEGRLLACSQCGQCYEPYCVSIKITKVVLS
KGWRCLECTVCEACGKATDPGRLLLCDDCDISYHTYCLDPPLQTVPKGGWKCKWCVWCRH
CGATSAGLRCEWQNNYTQCAPCASLSSCPVCYRNYREEDLILQCRQCDRWMHAVCQNLNT
EEEVENVADIGFDCSMCRPYMPASNVPSSDCCESSLVAQIVTKVKELDPPKTYTQDGVCL
TESGMTQLQSLTVTVPRRKRSKPKLKLKIINQNSVAVLQTPPDIQSEHSRDGEMDDSREG
ELMDCDGKSESSPEREAVDDETKGVEGTDGVKKRKRKPYRPGIGGFMVRQRSRTGQGKTK
RSVIRKDSSGSISEQLPCRDDGWSEQLPDTLVDESVSVTESTEKIKKRYRKRKNKLEETF
PAYLQEAFFGKDLLDTSRQSKISLDNLSEDGAQLLYKTNMNTGFLDPSLDPLLSSSSAPT
KSGTHGPADDPLADISEVLNTDDDILGIISDDLAKSVDHSDIGPVTDDPSSLPQPNVNQS
SRPLSEEQLDGILSPELDKMVTDGAILGKLYKIPELGGKDVEDLFTAVLSPANTQPTPLP
QPPPPTQLLPIHNQDAFSRMPLMNGLIGSSPHLPHNSLPPGSGLGTFSAIAQSSYPDARD
KNSAFNPMASDPNNSWTSSAPTVEGENDTMSNAQRSTLKWEKEEALGEMATVAPVLYTNI
NFPNLKEEFPDWTTRVKQIAKLWRKASSQERAPYVQKARDNRAALRINKVQMSNDSMKRQ
QQQDSIDPSSRIDSELFKDPLKQRESEHEQEWKFRQQMRQKSKQQAKIEATQKLEQVKNE
QQQQQQQQFGSQHLLVQSGSDTPSSGIQSPLTPQPGNGNMSPAQSFHKELFTKQPPSTPT
STSSDDVFVKPQAPPPPPAPSRIPIQDSLSQAQTSQPPSPQVFSPGSSNSRPPSPMDPYA
KMVGTPRPPPVGHSFSRRNSAAPVENCTPLSSVSRPLQMNETTANRPSPVRDLCSSSTTN
NDPYAKPPDTPRPVMTDQFPKSLGLSRSPVVSEQTAKGPIAAGTSDHFTKPSPRADVFQR
QRIPDSYARPLLTPAPLDSGPGPFKTPMQPPPSSQDPYGSVSQASRRLSVDPYERPALTP
RPIDNFSHNQSNDPYSQPPLTPHPAVNESFAHPSRAFSQPGTISRPTSQDPYSQPPGTPR
PVVDSYSQSSGTARSNTDPYSQPPGTPRPTTVDPYSQQPQTPRPSTQTDLFVTPVTNQRH
SDPYAHPPGTPRPGISVPYSQPPATPRPRISEGFTRSSMTRPVLMPNQDPFLQAAQNRGP
ALPGPLVRPPDTCSQTPRPPGPGLSDTFSRVSPSAARDPYDQSPMTPRSQSDSFGTSQTA
HDVADQPRPGSEGSFCASSNSPMHSQGQQFSGVSQLPGPVPTSGVTDTQNTVNMAQADTE
KLRQRQKLREIILQQQQQKKIAGRQEKGSQDSPAVPHPGPLQHWQPENVNQAFTRPPPPY
PGNIRSPVAPPLGPRYAVFPKDQRGPYPPDVASMGMRPHGFRFGFPGGSHGTMPSQERFL
VPPQQIQGSGVSPQLRRSVSVDMPRPLNNSQMNWPVGLPQHFSPQSLPVQQHNILGQAYI
ELRHRAPDGRQRLPFSAPPGSVVEASSNLRHGNFIPRPDFPGPRHTDPMRRPPQGLPNQL
PVHPDLEQVPPSQQEQGESVHSSSMVMRTLNHPLGGEFSEAPLSTSVPSETTSDNLQITT
QPSDGLEEKLDSDDPSVKELDVKDLEGVEVKDLDDEDLENLNLDTEDGKVVELDTLDNLE
TNDPNLDDLLRSGEFDIIAYTDPELDMGDKKSMFNEELDLPIDDKLDNQCVSVEPKKKEQ
ENKTLVLSDKHSPQKKSTVTNEVKTEVLSPNSKVESKCETEKNDENKDNVDTPCSQASAH
SDLNDGEKTSLHPCDPDLFEKRTNRETAGPSANVIQASTQLPAQDVINSCGITGSTPVLS
SLLANEKSDNSDIRPSGSPPPPTLPASPSNHVSSLPPFIAPPGRVLDNAMNSNVTVVSRV
NHVFSQGVQVNPGLIPGQSTVNHSLGTGKPATQTGPQTSQSGTSSMSGPQQLMIPQTLAQ
QNRERPLLLEEQPLLLQDLLDQERQEQQQQRQMQAMIRQRSEPFFPNIDFDAITDPIMKA
KMVALKGINKVMAQNNLGMPPMVMSRFPFMGQVVTGTQNSEGQNLGPQAIPQDGSITHQI
SRPNPPNFGPGFVNDSQRKQYEEWLQETQQLLQMQQKYLEEQIGAHRKSKKALSAKQRTA
KKAGREFPEEDAEQLKHVTEQQSMVQKQLEQIRKQQKEHAELIEDYRIKQQQQCAMAPPT
MMPSVQPQPPLIPGATPPTMSQPTFPMVPQQLQHQQHTTVISGHTSPVRMPSLPGWQPNS
APAHLPLNPPRIQPPIAQLPIKTCTPAPGTVSNANPQSGPPPRVEFDDNNPFSESFQERE
RKERLREQQERQRIQLMQEVDRQRALQQRMEMEQHGMVGSEISSSRTSVSQIPFYSSDLP
CDFMQPLGPLQQSPQHQQQMGQVLQQQNIQQGSINSPSTQTFMQTNERRQVGPPSFVPDS
PSIPVGSPNFSSVKQGHGNLSGTSFQQSPVRPSFTPALPAAPPVANSSLPCGQDSTITHG
HSYPGSTQSLIQLYSDIIPEEKGKKKRTRKKKRDDDAESTKAPSTPHSDITAPPTPGISE
TTSTPAVSTPSELPQQADQESVEPVGPSTPNMAAGQLCTELENKLPNSDFSQATPNQQTY
ANSEVDKLSMETPAKTEEIKLEKAETESCPGQEEPKLEEQNGSKVEGNAVACPVSSAQSP
PHSAGAPAAKGDSGNELLKHLLKNKKSSSLLNQKPEGSICSEDDCTKDNKLVEKQNPAEG
LQTLGAQMQGGFGCGNQLPKTDGGSETKKQRSKRTQRTGEKAAPRSKKRKKDEEEKQAMY
SSTDTFTHLKQVRQLSLLPLMEPIIGVNFAHFLPYGSGQFNSGNRLLGTFGSATLEGVSD
YYSQLIYKQNNLSNPPTPPASLPPTPPPMACQKMANGFATTEELAGKAGVLVSHEVTKTL
GPKPFQLPFRPQDDLLARALAQGPKTVDVPASLPTPPHNNQEELRIQDHCGDRDTPDSFV
PSSSPESVVGVEVSRYPDLSLVKEEPPEPVPSPIIPILPSTAGKSSESRRNDIKTEPGTL
YFASPFGPSPNGPRSGLISVAITLHPTAAENISSVVAAFSDLLHVRIPNSYEVSSAPDVP
SMGLVSSHRINPGLEYRQHLLLRGPPPGSANPPRLVSSYRLKQPNVPFPPTSNGLSGYKD
SSHGIAESAALRPQWCCHCKVVILGSGVRKSFKDLTLLNKDSRESTKRVEKDIVFCSNNC
FILYSSTAQAKNSENKESIPSLPQSPMRETPSKAFHQYSNNISTLDVHCLPQLPEKASPP
ASPPIAFPPAFEAAQVEAKPDELKVTVKLKPRLRAVHGGFEDCRPLNKKWRGMKWKKWSI
HIVIPKGTFKPPCEDEIDEFLKKLGTSLKPDPVPKDYRKCCFCHEEGDGLTDGPARLLNL
DLDLWVHLNCALWSTEVYETQAGALINVELALRRGLQMKCVFCHKTGATSGCHRFRCTNI
YHFTCAIKAQCMFFKDKTMLCPMHKPKGIHEQELSYFAVFRRVYVQRDEVRQIASIVQRG
ERDHTFRVGSLIFHTIGQLLPQQMQAFHSPKALFPVGYEASRLYWSTRYANRRCRYLCSI
EEKDGRPVFVIRIVEQGHEDLVLSDISPKGVWDKILEPVACVRKKSEMLQLFPAYLKGED
LFGLTVSAVARIAESLPGVEACENYTFRYGRNPLMELPLAVNPTGCARSEPKMSAHVKRP
HTLNSTSTSKSFQSTVTGELNAPYSKQFVHSKSSQYRKMKTEWKSNVYLARSRIQGLGLY
AARDIEKHTMVIEYIGTIIRNEVANRKEKLYESQNRGVYMFRMDNDHVIDATLTGGPARY
INHSCAPNCVAEVVTFERGHKIIISSSRRIQKGEELCYDYKFDFEDDQHKIPCHCGAVNC
RKWMN
>gi|21359851|gb|NM_000966.2|RARG 2663 bp mRNA Homo sapiens
retinoic acid receptor, gamma (RARG), mRNA.
GGCACGAGGCAGTGGGCAGGCCAGGCAGGGCGGGTACGGAGCCTCCCAGGCTGGGGCAGT
GGGCATGGGCAGGGGCTGTGGCTGAAGACCTCGCCCGCCCACTGCAGACTCCAGGGGACT
CTCACACCGCAGCTGCCATGGCCACCAATAAGGAGCGACTCTTTGCGGCTGGTGCCCTGG
GGCCTGGATCTGGCTACCCAGGGGCAGGTTTCCCCTTCGCCTTCCCAGGGGCACTCAGGG
GGTCTCCGCCTTTCGAGATGCTGAGCCCTAGCTTCCGGGGCCTGGGCCAGCCTGACCTCC
CCAAGGAGATGGCCTCTCTGTCGGTGGAGACACAGAGCACCAGCTCAGAGGAGATGGTGC
CCAGCTCGCCCTCGCCCCCTCCGCCTCCTCGGGTCTACAAGCCATGCTTCGTGTGCAATG
ACAAGTCCTCTGGCTACCACTATGGGGTCAGCTCTTGTGAAGGCTGCAAGGGCTTCTTTC
GCCGAAGCATCCAGAAGAACATGGTGTACACGTGTCACCGCGACAAAAACTGTATCATCA
ACAAGGTGACCAGGAATCGCTGCCAGTACTGCCGGCTACAGAAGTGCTTCGAAGTGGGCA
TGTCCAAGGAAGCTGTGCGAAATGACCGGAACAAGAAGAAGAAAGAGGTGAAGGAAGAAG
GGTCACCTGACAGCTATGAGCTGAGCCCTCAGTTAGAAGAGCTCATCACCAAGGTCAGCA
AAGCCCATCAGGAGACTTTCCCCTCGCTCTGCCAGCTGGGCAAGTATACCACGAACTCCA
GTGCAGACCACCGCGTGCAGCTGGATCTGGGGCTGTGGGACAAGTTCAGTGAGCTGGCTA
CCAAGTGCATCATCAAGATCGTGGAGTTTGCCAAGCGGTTGCCTGGCTTTACAGGGCTCA
GCATTGCTGACCAGATCACTCTGCTCAAAGCTGCCTGCCTAGATATCCTGATGCTGCGTA
TCTGCACAAGGTACACCCCAGAGCAGGACACCATGACCTTCTCCGACGGGCTGACCCTGA
ACCGGACCCAGATGCACAATGCCGGCTTCGGGCCCCTCACAGACCTTGTCTTTGCCTTTG
CTGGGCAGCTCCTGCCCCTGGAGATGGATGACACCGAGACAGGGCTGCTCAGCGCCATCT
GCCTCATCTGCGGAGACCGCATGGACCTGGAGGAGCCCGAAAAAGTGGACAAGCTGCAGG
AGCCACTGCTGGAAGCCCTGAGGCTGTACGCCCGGCGCCGGCGGCCCAGCCAGCCCTACA
TGTTCCCAAGGATGCTAATGAAAATCACCGACCTCCGGGGCATCAGCACTAAGGGAGCTG
AAAGGGCCATTACTCTGAAGATGGAGATTCCAGGCCCGATGCCTCCCTTAATCCGAGAGA
TGCTGGAGAACCCTGAAATGTTTGAGGATGACTCCTCGCAGCCTGGTCCCCACCCCAATG
CCTCTAGCGAGGATGAGGTTCCTGGGGGCCAGGGCAAAGGGGGCCTGAAGTCCCCAGCCT
GACCAGGGCCCCTGACCTCCCCGCTGTGGGGGTTGGGGCTTCAGGCAGCAGACTGACCAT
CTCCCAGACCGCCAGTGACTGGGGGAGGACCTGCTCTGCCCTCTCCCCACCCCTTCCAAT
GAGCTCCTTGTTTTTGCCAAAGTTTCTAGGGGTGCCTCTGTGTTCATCCCCTTCCTGATC
TAACCGGCTCCCTCGCCAGTCCCGGGGGCCTGCCCTGCTCCCACCAGGAGAGAGGGCAAA
GGGATGAGCCTGGGTTTGGACTCTAAAATCTCAGCACTGCCCCATGGGTCCTAGACTTCC
CAGGGCAAGAGGAAGACCCTGCCATTCCACAGCCCCTTCCTCTGCCAGGTGCTTGGCTCT
CTGAGAGCAAACAGGAACACTAGAGACCAAAAAGGGGACAAAGGAGAAGGGCTGAGCCCA
CCTTCTTGCTCCTACCCTTGGTGCCTAATGCTGTGTGATGCACCTGCAGGGTGTGTGCTA
GCCTCTGTGCCCCGTCCTTGTGCCAGGTCAAGGTGGGGGCAGGCTGGGCCCTGCATTTCT
GGGGCAGGAACAGAGGGTGAAAGGGACAGATAGATGCAGGTCCATTCTGCACCTCTTGGC
TCGGGTGCAGAGTTCACCCTGTGCCCTCCGTTATAAGTCCCTCCCCCAGCCCTGTCATGT
GCCTTGGGCTCCTCCTGCCCTCCATCTCAGCCATTGGGGCAGGGACCCTCCTACACTACA
GAGGGGCCAGGGGATCCCTCTCTCCCTAGTGCCTTCCACCCTTTACTCCCCAGAGCAGCT
TGGCCCAGGGAGGGGGGATGCTGCTTAGCTGATCCCGCCCTGACCCAGAGGAAGCCTCTA
TTTATTTATTAGCTTTTGTTTACACCGTGGAATTGACCCCTTCCTCCAGGGGTCTTGGGT
GGGGGAGCCCAGGGCCCCTGTGACCCCTCCTTTCTTCCTCCAATCCCCAGTTTGTATTTA
GCTGCCAAATAAGATTCCCATTGGCTCCCTGTGTTCTCTTGGGGGGTCAGGGTGCTGTCC
CCTCCCCTCTGTTTACATCTCCCCTCTACCCCGCTGTATCGCATATTGCTGAGTTTTCTA
TTTTTGCAAAATAAAGTGATGGAAACTCAAAAAAAAAAAAAAAAAAAAAAAAAAAAAAAA
AAAAAAAAAAAAAAAAAAAAAAA
>gi|4506423|gb|NP_000957.1|RARG 454 aa linear retinoic acid
receptor, gamma; Retinoic acid receptor, gamma polypeptide
[Homo sapiens].
MATNKERLFAAGALGPGSGYPGAGFPFAFPGALRGSPPFEMLSPSFRGLGQPDLPKEMAS
LSVETQSTSSEEMVPSSPSPPPPPRVYKPCFVCNDKSSGYHYGVSSCEGCKGFFRRSIQK
NMVYTCHRDKNCIINKVTRNRCQYCRLQKCFEVGMSKEAVRNDRNKKKKEVKEEGSPSDY
ELSPQLEELITKVSKAHQETFPSLCQLGKYTTNSSADHRVQLDLGLWDKFSELATKCIIK
IVEFAKRLPGFTGLSIADQITLLKAACLDILMLRICTRYTPEQDTMTFSDGLTLNRTQMH
NAGFGPLTDLVFAFAGQLLPLEMDDTETGLLSAICLICGDRMDLEEPEKVDKLQEPLLEA
LRLYARRRRPSQPYMFPRMLMKITDLRGISTKGAERAITLKMEIPGPMPPLIREMLENPE
MFEDDSSQPGPHPNASSEDEVPGGQGKGGLKSPA
>gi|14670376|gb|NM_015318.1|P114-RHO-GEF 5113 bp mRNA Homo
sapiens Rho-specific guanine nucleotide exchange factor p114
(P114-RHO-GEF), mRNA.
GCTGGCGGAGAGCGGCCTGCGGGCGATCGGGCCGAGCCTCGCTCAAGGAGCACCCCCGGG
GCACCCTCCTGTCCGATGGCAGCCCGGCCCTGTCCAGGAATGTCGGTATGACGGTCTCTC
AGAAAGGGGGTCCCCAGCCAACACCGAGCCCGGCTGGCCCTGGGACGCAACTCGGACCAA
TCACAGGAGAGATGGATGAAGCCGATTCTGCGTTTTTAAAATTTAAGCAGACAGCTGATG
ACTCTCTGTCCCTTACATCTCCAAACACCGAGTCCATTTTTGTAGAAGATCCCTACACCG
CCTCGCTGAGGAGTGAGATTGAGTCAGACGGCCACGAGTTTGAAGCTGAGTCCTGGAGCC
TCGCCGTGGATGCAGCCTACGCCAAGAAGCAAAAGAGGGAGGTGGTGAAAAGACAAGATG
TCCTTTATGAGCTGATGCAGACAGAGGTGCACCACGTGCGGACGCTCAAGATCATGCTGA
AGGTGTACTCCAGGGCCCTGCAGGAGGAGCTGCAGTTCAGCAGCAAGGCCATTGGCCGCC
TCTTCCCATGCGCTGACGACCTGCTGGAGACGCACAGCCACTTCCTCGCTCGGCTCAAGG
AGCGCCGCCAGGAGTCCCTGGAGGAGGGCAGTGACCGGAATTATGTCATCCAGAAAATCG
GCGACCTCCTGGTTCAGCAGTTTTCAGGTGAAAATGGGGAGAGAATGAAAGAAAAGTACG
GTGTGTTTTGTAGTGGCCACAATGAAGCTGTTAGTCATTACAAGTTGCTGCTTCAGCAAA
ACAAGAAATTTCAAAACTTGATCAAGAAAATTGGCAACTTCTCCATCGTGCGGCGGCTTG
GCGTGCAGGAGTGCATTCTCCTGGTTACACAACGCATAACCAAATACCCAGTGCTGGTGG
AGCGCATCATCCAGAACACGGAAGCTGGCACTGAGGACTATGAAGACCTGACCCAGGCCT
TGAACCTCATCAAAGATATCATCTCACAAGTGGACGCCAAGGTCAGTGAGTGTGAGAAGG
GCCAGCGCCTCAGGGAGATCGCAGGGAAGATGGACCTGAAGTCTTCCAGCAAACTCAAGA
ACGGGCTCACCTTCCGCAAGGAAGACATGCTTCAGCGGCAGCTCCACCTGGAGGGCATGC
TATGCTGGAAGACCACATCAGGGCGCTTGAAAGATATCCTGGCTATCCTGCTGACCGACG
TACTTTTGCTGCTACAAGAAAAAGATCAGAAATACGTCTTTGCTTCTGTGGACTCAAAGC
CACCCGTCATCTCGTTACAAAAGCTCATCGTGAGGGAAGTGGCCAACGAGGAGAAAGCGA
TGTTTCTGATCAGCGCCTCCTTGCAAGGGCCGGAGATGTATGAAATCTACACGAGCTCCA
AAGAGGACAGGAACGCCTGGATGGCCCACATCCAAAGGGCTGTGGAGAGCTGCCCTGACG
AGGAGGAGGGGCCCTTCAGCCTGCCCGAAGAGGAAAGGAAGGTGGTCGAGGCCCGCGCCA
CGAGACTCCGGGACTTTCAAGAGCGGTTGAGCATGAAAGACCAGCTGATCGCACAGAGCC
TCCTAGAGAAACAGCAGATCTACCTGGAGATGGCCGAGATGGGCGGCCTCGAAGACCTGC
CCCAGCCCCGAGGCCTATTCCGTGGAGGGGACCCATCCGAGACCCTGCAGGGGGAGCTAA
TTCTCAAGTCGGCCATGAGCGAGATCGAGGGCATCCAGAGCCTGATCTGCAGGCGGCTGG
GCAGCGCCAACGGCCAGGCGGAAGACGGAGGCAGCTCCACAGGCCCGCCCAGGAGGGCTG
AGACCTTCGCGGGCTACGACTGCACAAACAGCCCCACCAAGAATGGCAGTTTCAAGAAGA
AAGTCAGCAGCACTGACCCCAGGCCCCGAGACTGGCGAGGCCCCCCAAACAGCCCGGACT
TGAAGCTCAGTGACAGTGACATTCCTGGGAGCTCTGAGGAATCGCCGCAGGTGGTGGAGG
CGCCAGGCACGGAATCCGATCCCCGTCTGCCCACCGTCCTGGAGTCGGAGCTTGTCCAGC
GGATCCAGACACTGTCCCAGCTGCTCCTGAACCTTCAGGCGGTAATCGCCCACCAGGACA
GCTATGTGGAGACGCAGCGGGCTGCCATCCAGGAGCGGGAGAAGCAGTTCCGGCTGCAGT
CGACGCGTGGGAACCTGCTGCTGGAGCAGGAGCGGCAACGCAACTTCGAGAAGCAGCGGG
AGGAGCGCGCGGCCCTGGAGAAGCTGCAGAGCCAGCTGCGGCACGAGCAGCAGCGCTGGG
AGCGCGAGCGCCAGTGGCAGCACCAGGAGCTGGAGCGTGCGGGCGCGCGGCTGCAGGAGC
GCGAGGGCGAGGCGCGGCAGCTACGCGAGCGGCTGGAGCAGGAGCGGGCCGAGCTGGAGC
GCCAGCGCCAGGCCTACCAGCACGACCTGGAGCGGCTGCGCGAGGCCCAGCGTGCCGTGG
AGCGCGAGCGGGAGCGCCTGGAGCTGCTGCGCCGCCTCAAGAAGCAGAACACCGCGCCAG
GCGCGCTGCCGCCCGACACACTGGCCGAGGCCCAGCCCCCAAGCCACCCTCCCAGCTTCA
ACGGGGAAGGGCTGGAGGGCCCTCGGGTGAGCATGCTGCCATCCGGCGTGGGGCCAGAGT
ACGCAGAGCGCCCCGAGGTGGCTCGCCGGGACAGCGCCCCCACCGAGAGCCGGCTGGCCA
AGAGCGATGTGCCCATCCAGCTGCTCAGCGCCACCAACCAGTTCCAGAGGCAGGCGGCCG
TGCAGCAGCAGATCCCCACCJAGCTGGCGGCCTCCACCAAGGGTGGCAAGGACAAGGGCG
GCAAGAGCAGGGGCTCTCAGCGCTGGGAGAGCTCAGCGTCCTTCGACCTGAAGCAGCAGC
TGCTGCTCAACAAGCTCATGGGGAAAGATGAGAGCACCTCACGGAACCGCCGCTCGCTGA
GCCCTATCCTGCCCGGCAGACACAGTCCTGCGCCCCCACCAGACCCTGGCTTCCCCGCCC
CGAGCCCACCGCCAGCTGACAGCCCCTCCGAGGGCTTCTCTCTCAAGGCCGGGGGCACAG
CCCTCCTGCCCGGCCCCCCAGCTCCCTCGCCACTGCCGGCCACACCACTCAGCGCCAAGG
AGGACGCCAGCAAAGAAGACGTCATCTTCTTCTAAAAGGGCCGTGACTCAAGGAAAGTTT
TTAATGGAAAGTTGAGCCAGAACTAAACCAGGGAGCTGTCTGAAATCATAGCACCCCATC
CGGGTGGCGGGGAGATCAACTCCGAGCTGTTTTTCCGAGGCAGTGAGGAACGGTGCCGGC
TCTGCACGGAGCTGAGGACAGGACAGACCTTGCTTTGAGAAGGAGCTGCCGGCCGGGGCC
ACGCTCCACAGCCGCCGCGCGACAGTGGAGCCAAGGGTTAGGGCACCAGGAGGGGCCAGG
TGGCGTCGGCAGCATCTGTCCCCAGAATCAGGCAGAATCCACTTCCCAAACAGAGCCCCA
CGCAGGTTCACCATGAACCTCAGGGTCAGGGAATGAGCCAGGCACGGGGGCATGGGCAGA
GAGGGCCACGGGGCAGGGCCCACTGAGGGAACATCAGTGGCCCTCCAGTCAGGTTCTGTG
GGTTTGGAAGCCCATCGTGAAAGGGGCTGACCTTTGCCCCTTTTTACTTGGCATTGGTTT
TGAAACCAGCTGTTTCCCAAACTCTGCTTCCCAAGGGCAACCGTTGCTGTTCACACGCTC
AGCCTGTCTGGGGGAGCGGGCCTCTAGCTTCAGCCAGGGCGGGTACACACCCTGGGCACA
GGGTCCTCAGCCCCCGGGAAATGAGCTCCCAGGGCTGGCGTCCCACCTTCCAGGTGGGGG
CTGGCACATCACAGACTGTCGAGAGCGCCATGTCCCAGGGCATGCAGAGGTTGCACCTAG
AGACGTTGCAGCAAGTGGACAAGTGGCCGCTGTGCGGGCCCCTCGCTTGTAGTGAGCTGT
TGCAGCTTACGGTCCGTTCCCTGGAGGGGTGGAGGAAGGAGGTGTTGGGCAGCATCAAAG
GTGCTGGGACATCCCAGGGTGGTGAGATCCATCCACGATCCAGCTCCGGTGGAGAAAGGG
CCCATGTCAAGCCTTGTTCTGCACCCCAAGCATTGGTGGTAGGACTGGGTCCTGGCTGAT
CGTCCTTGTTCCCAGTGGGGTACATGTGAGCCCCTGCCAGGGCCAAGTCCTTCTCCCGAA
CCCAGGGTCCTGGGAACTGCAGATCCCGGGGGGATTCAGCCCTTCTCCCACTGTGCTGGC
AGAGGCACTCCTGTGACGCTGAATACAGTGAACAGGGACATTCCCGCCACTCGGGGACAG
ATGGGCACAAGGGAGGGGAAACTCCATCAGGAAGTGCTCCCCTGGGCAGAGGCGCCCACT
GGGTGCTGTGGGCTCAGGAGGGGGCGGGGCAGGAGCTGGTGCCAACCGGGAACCAGAGCC
CCACAGCCATACAGCCCATTGGTGACAAGGTCCTGAGAACACAGTGGCCAGGTGTCCCCA
GGCTCCTGGCCCCTCCGACGACCTCAACTCTGCCCAGCCCGGTCCCTGGCCATCAGCGAC
GCTGTCCGCCCCCCGTCAGATCCCATGTGTGCCATGTTTATCATCAGTGTTTTGTATTTT
TGTACTGAGTATCGGAGCACTTTACAGAAGCTGACTGTACATTCCTGTTCTGTTGTGAAG
AGAACATTCCCAGACCCTGGCACCCTCCTGAGCCGGCGTGTGCCGGTCCAGCCCTCCGAG
ATGCCACAATTCCTTGGATGGGGGAGAAGTTCAAGGAATTTCTGCTCGGCCACGCGGTGG
GAACCCCGCGTCCCCGCCATGTGGCAGAGGGGTCTCAGTCGTGCTAGGCATCGGGCGGCA
GCGCCGACAGCCCTTCCCTCGCCAGTGCCCCTCGGCCACTCCTGGGTTGGAGCCCGATTT
TATTTGTAAAGTTGACAGTCGAGCAAATGTTCCTATTTTCGTGGGATCTGCACACGTCTT
TGTCAGTTGTGGTCATGATCTTAGTCACCTGCTAATTATTTTTACAATGATTACAACATT
TCCTCACTGCGGGATATTTCTGACCCGCTTTAGAACTTAAGACCTGATTCTAGCAATAAA
CGTGTCCGAGATG
>gi|14670377|gb|NP_056133.1|P114-RHO-GEF 1015 aa linear Rho-
specific guanine nucleotide exchange factor p114 [Homo
sapiens].
MTVSQKGGPQPTPSPAGPGTQLGPITGEMDEADSAFLKFKQTADDSLSLTSPNTESIFVE
DPYTASLRSEIESDGHEFEAESWSLAVDAAYAKKQKREVVKRQDVLYELMQTEVHHVRTL
KIMLKVYSRALQEELQFSSKAIGRLFPCADDLLETHSHFLARLKERRQESLEEGSDRNYV
IQKIGDLLVQQFSGENGERMKEKYGVFCSGHNEAVSHYKLLLQQNKKFQNLIKKIGNFSI
VRRLGVQECILLVTQRITKYPVLVERIIQNTEAGTEDYEDLTQALNLIKDIISQVDAKVS
ECEKGQRLREIAGKMDLKSSSKLKNGLTFRKEDMLQRQLHLEGMLCWKTTSGRLKDILAI
LLTDVLLLLQEKDQKYVFASVDSKPPVISLQKLIVREVANEEKAMFLISASLQGPEMYEI
YTSSKEDRNAWMAHIQRAVESCPDEEEGPFSLPEEERKVVEARATRLRDFQERLSMKDQL
IAQSLLEKQQIYLEMAEMGGLEDLPQPRGLFRGGDPSETLQGELILKSAMSEIEGIQSLI
CRRLGSANGQAEDGGSSTGPPRRAETFAGYDCTNSPTKNGSFKKKVSSTDPRPRDWRGPP
NSPDLKLSDSDIPGSSEESPQVVEAPGTESDPRLPTVLESELVQRIQTLSQLLLNLQAVI
AHQDSYVETQRAAIQEREKQFRLQSTRGNLLLEQERQRNFEKQREERAALEKLQSQLRHE
QQRWERERQWQHQELERAGARLQEREGEARQLRERLEQERAELERQRQAYQHDLERLREA
QRAVERERERLELLRRLKKQNTAPGALPPDTLAEAQPPSHPPSFNGEGLEGPRVSMLPSG
VGPEYAERPEVARRDSAPTESRLAKSDVPIQLLSATNQFQRQAAVQQQIPTKLAASTKGG
KDKGGKSRGSQRWESSASFDLKQQLLLNKLMGKDESTSRNRRSLSPILPGRHSPAPPPDP
GFPAPSPPPADSPSEGFSLKAGGTALLPGPPAPSPLPATPLSAKEDASKEDVIFF
>gi|23238259|gb|NM_005198.3|CHKL 1595 bp mRNA Homo sapiens
choline kinase-like (CHKL), transcript variant 1, mRNA.
CCCGGGCCGGGGCACGGAGAGAGCCGAGCGCCGCAGCCGTGAGCCGAATAGAGCCGGAGA
GACCCGAGTATGACCGGAGAAGCCCAGGCCGGCCGGAAGAGGAGCCGAGCGCGGCCGGAA
GGAACCGAGCCCGTCCGAAGGGAGCGGAGCGCAGCCTGGCCTGGGGCCCGGTCGAGCCCG
CGCCATGGCGGCCGAGGCGACAGCTGTGGCCGGAAGCGGGGCTGTTGGCGGCTGCCTGGC
CAAAGACGGCTTGCAGCAGTCTAAGTGCCCGGACACTACCCCAAAACGGCGGCGCGCCTC
GTCGCTGTCGCGTGACGCCGAGCGCCGAGCCTACCAATGGTGCCGGGAGTACTTGGGCGG
GGCCTGGCGCCGAGTGCAGCCCGAGGAGCTGAGGGTTTACCCCGTGAGCGGAGGCCTCAG
CAACCTGCTCTTCCGCTGCTCGCTCCCGGACCACCTGCCCAGCGTTGGCGAGGAGCCCCG
GGAGGTGCTTCTGCGGCTGTACGGAGCCATCTTGCAGGGCGTGGACTCCCTGGTGCTAGA
AAGCGTGATGTTCGCCATACTTGCGGAGCGGTCGCTGGGGCCCCAGCTGTACGGAGTCTT
CCCAGAGGGCCGGCTGGAACAGTACATCCCAAGTCGGCCATTGAAAACTCAAGAGCTTCG
AGAGCCAGTGTTGTCAGCAGCCATTGCCACGAAGATGGCGCAATTTCATGGCATGGAGAT
GCCTTTCACCAAGGAGCCCCACTGGCTGTTTGGGACCATGGAGCGGTACCTAAAACAGAT
CCAGGACCTGCCCCCAACTGGCCTCCCTGAGATGAACCTGCTGGAGATGTACAGCCTGAA
GGATGAGATGGGCAACCTCAGGAAGTTACTAGAGTCTACCCCATCGCCAGTCGTCTTCTG
CCACAATGACATCCAGGAAGGGAACATCTTGCTGCTCTCAGAGCCAGAAAATGCTGACAG
CCTCATGCTGGTGGACTTCGAGTACAGCAGTTATAACTATAGGGGCTTTGACATTGGGAA
CCATTTTTGTGAGTGGGTTTATGATTATACTCACGAGGAATGGCCTTTCTACAAAGCAAG
GCCCACAGACTACCCCACTCAAGAACAGCAGTTGCATTTTATTCGTCATTACCTGGCAGA
GGCAAAGAAAGGTGAGACCCTCTCCCAAGAGGAGCAGAGAAAACTGGAAGAAGATTTGCT
GGTAGAAGTCAGTCGGTATGCTCTGGCATCCCATTTCTTCTGGGGTCTGTGGTCCATCCT
CCAGGCATCCATGTCCACCATAGAATTTGGTTACTTGGACTATGCCCAGTCTCGGTTCCA
GTTCTACTTCCAGCAGAAGGGGCAGCTGACCAGTGTCGACTCCTCATCCTGACTCCACCC
TCCCACTCCTTGGATTTCTCCTGGAGCCTCCAGGGCAGGACCTTGGAGGGAGGAACAACG
AGCAGAAGGCCCTGGCGACTGGGCTGAGCCCCCAAGTGAAACTGAGGTTCAGGAGACCGG
CCTGTTCCTGAGTTTGAGTAGGTCCCCATGGCTGGCAGGCCAGAGCCCCGTGCTGTGTAT
GTAACACAATAAACAAGCTTCTTCTTCCCACCCTG
>gi|6978649|gb|NP_005189.2|CHKL 395 aa linear choline/
ethanolamine kinase isoform a [Homo sapiens].
MAAEATAVAGSGAVGGCLAKDGLQQSKCPDTTPKRRPASSLSRDAERRAYQWCREYLGGA
WRRVQPEELRVYPVSGGLSNLLFRCSLPDHLPSVGEEPREVLLRLYGAILQGVDSLVLES
VMFAILAERSLGPQLYGVFPEGRLEQYIPSRPLKTQELREPVLSAAIATKMAQFHGMEMP
FTKEPHWLFGTMERYLKQIQDLPPTGLPEMNLLEMYSLKDEMGNLRKLLESTPSPVVFCH
NDIQEGNILLLSEPENADSLMLVDFEYSSYNYRGFDIGNHFCEWVYDYTHEEWPFYKARP
TDYPTQEQQLHFIRHYLAEAKKGETLSQEEQRKLEEDLLVEVSRYALASHFFWGLWSILQ
ASMSTIEFGYLDYAQSRFQFYFQQKGQLTSVHSSS
>gi|4757755|gb|NM_004039.1|ANXA2 1362 bp mRNA Homo sapiens
annexin A2 (ANXA2), mRNA.
CATTTGGGGACGCTCTCAGCTCTCGGCGCACGGCCCAGCTTCCTTCAAAATGTCTACTGT
TCACGAAATCCTGTGCAAGCTCAGCTTGGAGGGTGATCACTCTACACCCCCAAGTGCATA
TGGGTCTGTCAAAGCCTATACTAACTTTGATGCTGAGCGGGATGCTTTGAACATTGAAAC
AGCCATCAAGACCAAAGGTGTGGATGAGGTCACCATTGTCAACATTTTGACCAACCGCAG
CAATGCACAGAGACAGGATATTGCCTTCGCCTACCAGAGAAGGACCAAAAAGGAACTTGC
ATCAGCACTGAAGTCAGCCTTATCTGGCCACCTGGAGACGGTGATTTTGGGCCTATTGAA
GACACCTGCTCAGTATGACGCTTCTGAGCTAAAAGCTTCCATGAAGGGGCTGGGAACCGA
CGAGGACTCTCTCATTGAGATCATCTGCTCCAGAACCAACCAGGAGCTGCAGGAAATTAA
CAGAGTCTACAAGGAAATGTACAAGACTGATCTGGAGAAGGACATTATTTCGGACACATC
TGGTGACTTCCGCAAGCTGATGGTTGCCCTGGCAAAGGGTAGAAGAGCAGAGGATGGCTC
TGTCATTGATTATGAACTGATTGACCAAGATGCTCGGGATCTCTATGACGCTGGAGTGAA
GAGGAAAGGAACTGATGTTCCCAAGTGGATCAGCATCATGACCGAGCGGAGCGTGCCCCA
CCTCCAGAAAGTATTTGATAGGTACAAGAGTTACAGCCCTTATGACATGTTGGAAAGCAT
CAGGAAAGAGGTTAAAGGAGACCTGGAAAATGCTTTCCTGAACCTGGTTCAGTGCATTCA
GAACAAGCCCCTGTATTTTGCTGATCGGCTGTATGACTCCATGAAGGGCAAGGGGACGCG
AGATAAGGTCCTGATCAGAATCATGGTCTCCCGCAGTGAAGTGGACATGTTGAAAATTAG
GTCTGAATTCAAGAGAAAGTACGGCAAGTCCCTGTACTATTATATCCAGCAAGACACTAA
GGGCGACTACCAGAAAGCGCTGCTGTACCTGTGTGGTGGAGATGACTGAAGCCCGACACG
GCCTGAGCGTCCAGAAATGGTGCTCACCATGCTTCCAGCTAACAGGTCTAGAAAACCAGC
TTGCGAATAACAGTCCCCGTGGCCATCCCTGTGAGGGTGACGTTAGCATTACCCCCAACC
TCATTTTAGTTGCCTAAGCATTGCCTGGCCTTCCTGTCTAGTCTCTCCTGTAAGCCAAAG
AAATGAACATTCCAAGGAGTTGGAAGTGAAGTCTATGATGTGAAACACTTTGCCTCCTGT
GTACTGTGTCATAAACAGATGAATAAACTGAATTTGTACTTT
>gi|4757756|gb|NP_004030.1|ANXA2 339 aa linear annexin A2;
annexin II; annexin II (lipocortin II); calpactin I, heavy
polypeptide (p36); lipocortin II; Annexin II (lipocortin I);
annexin II (lipocortin II; calpactin I, heavy polypeptide)
[Homo sapiens].
MSTVHEILCKLSLEGDHSTPPSAYGSVKAYTNFDAERDALNIETAIKTKGVDEVTIVNIL
TNRSNAQRQDIAFAYQRRTKKELASALKSALSGHLETVILGLLKTPAQYDASELKASMKG
LGTDEDSLIEIICSRTNQELQEINRVYKEMYKTDLEKDIISDTSGDFRKLMVALAKGRRA
EDGSVIDYELIDQDARDLYDAGVKRKGTDVPKWISIMTERSVPHLQKVFDRYKSYSPYDM
LESIRKEVKGDLENAFLNLVQCIQNKPLYFADRLYDSMKGKGTRDKVLIRIMVSRSEVDM
LKIRSEFKRKYGKSLYYYIQQDTKGDYQKALLYLCGGDD
>gi|27484939|gb|XM_084635.3|LOC143785 1982 bp mRNA Homo
sapiens similar to hypothetical protein XP_084635 [Homo
sapiens](LOC143785), mRNA.
TACTATCAGGGGGCAAGAGCCTTTCTCTCCAGCTACACACTCCATCTCCCGGGAGCAAGG
GGAAACTCCGAGAGGAGGGCAACAGAGCCAGCATCTTGCCAGGGCCCCGGAGGAGGGGTT
CCCCGCTACGCCTGTGCCGGAGGAGTTCCAGTCACCGAGCGAGGGGCGCAAGGGTGGGTG
CATCCTGCGCTGCGGCGGGCGCGCTACCCAGACGCTGGTGTGCAGAGCCACATGAAGCCT
GCTGGGGACTGGGGGCCAGGGAGCAGCAAGCCAGCTGGGACTGAGGCGGACGCTGTCTCA
GGGAGACGCTGACTCGCAAAGACACTCCCTTCCTTGTGCCTGGGTAAAAAGTCTCCTCCT
GGGGTCCCTGGCCATCCTGAATATCCAGAATGGTGTTTCTGAAGTTCTTCTGCATGAGTT
TCTTCTGCCACCTGTGTCAAGGCTACTTCGATGGCCCCCTCTACCCAGAGATGTCCAATG
GGACTCTGCACCACTACTTCGTGCCCGATGGGGACTATGAGGAGAACGATGACCCCGAGA
AGTGCCAGCTGCTCTTCAGGGTGAGTGACCACAGGCGCTGCTCCCAGGGGGAGGGGAGCC
AGGTTGGCAGCCTGCTGAGCCTCACCCTGCGGGAGGAGTTCACCGTGCTGGGCCGCCAGG
TGGAGGATGCTGGGCGCGTGCTGGAGGGCATCAGCAAAAGCATCTCCTACGACCTAGACG
GGGAAGAGAGCTATGGCAAGTACCTGCGGCGGGAGTCCCACCAGATCGGGGATGCCTACT
CCAACTCGGACAAATCCCTCACTGAGCTGGAGAGCAAGTTCAAGCAGGGCCAGGAACAGG
ACAGCCGGCAGGAGAGCAGGCTCAACGAGGACTTTCTGGGAATGCTGGTCCACACCAGGT
CCCTGCTGAAGGAGACACTGGACATCTCTGTGGGGCTCAGGGACAAATACGAGCTGCTGG
CCCTCACCATTAGGAGCCATGGGACCCGACTAGGTCGGCTGAAAAATGATTATCTTAAAG
TATAGGTGGAAGGATACAAATGCTAGAAAGAGGGAATCAAATCAGCCCCGTTTTGGAGGG
TGGGGGACAGAAGATGGGGCTACATTTCCCCCATACCTACTATTTTTTTATATCCCGATT
TGCACTTTGAGAATACATCTAAGGTCATCTTTCAAAAGAGAAAAATTGGACACTTGAGTG
ACTTTGTTTTTAGTTTTGTTTTTGTACATTATTTATGTGATTGTTATGGAATTGTCACCT
GGAAAGAACAATTTTAAGCAATGTCATTTCTAGATGGGTTTCTAATTCTGCAGAGACACC
CGTTTCAGCCACATCTAAAAGAGCACAGTTTATGTGGTGCGGAATTAAACTTCCCCATCC
TGCAGATTATGTGGAAATACCCAAAGATAATAGTGCATAGCTCCTTTCAGCCTCTAGCCT
TCACTCCTGGGCTCCAAAAGCTATCCCAGTTGCCTGTTTTTCAAATGAGGTTCAAGGTGC
TGCTTTGCATGCCTGCCAACCCATGGAAGTTGTTTCTTACTTCTTTTCTCTCTTATTTAT
TAACCATGGTCTGAGAGTTGTTTTTGTTCTATGTAACAGTATTGCCACAAAACTATAGGC
AAATCGTGTTTGCAGGGAGATTTCTGATGCCTCTGTGGGTGTGTGTAAGTTAAAGTGGCC
ACATTTAAGAAGGCCAAGCTTTGTAGTGGTTGCACAGTCACACTGATATGCTGATTTGCT
CTTTCTCATTGTATGTCTATGCTTTGTCATCAGTGCTATAGTAAATTACAAAGAAATAGG
TAGATTGTATGAACATACCCACAAATGCCTATGATTTAGGTTACCAATGTATTCTTTCTC
ATTTGGGGTTTTGCTTCTGTCTGTCTGTTTATTGGAAACTTGTACTTCAAGTAGGGGGAA
TCCTAATTCTAATAACTCCTTAGCTAAGTTTTATTATTCAGGCAATAAACATGTTTTCAT
GT
>gi|18578340|gb|XP_084635.1|LOC143785 211 aa linear similar
to hypothetical protein XP_084635 [Homo sapiens].
MVFLKFFCMSFFCHLCQGYFDGPLYPEMSNGTLHHYFVPDGDYEENDDPEKCQLLFRVSD
HRRCSQGEGSQVGSLLSLTLREEFTVLGRQVEDAGRVLEGISKSISYDLDGEESYGKYLR
RESHQIGDAYSNSDKSLTELESKFKQGQEQDSRQESRLNEDFLGMLVHTRSLLKETLDIS
VGLRDKYELLALTIRSHGTRLGRLKNDYLKV
>gi|4507464|gb|NM_003239.1|TGFB3 2574 bp mRNA Homo sapiens
transforming growth factor, beta 3 (TGFB3), mRNA.
CCTGTTTAGACACATGGACAACAATCCCAGCGCTACAAGGCACACAGTCCGCTTCTTCGT
CCTCAGGGTTGCCAGCGCTTCCTGGAAGTCCTGAAGCTCTCGCAGTGCAGTGAGTTCATG
CACCTTCTTGCCAAGCCTCAGTCTTTGGGATCTGGGGAGGCCGCCTGGTTTTCCTCCCTC
CTTCTGCACGTCTGCTGGGGTCTCTTCCTCTCCAGGCCTTGCCGTCCCCCTGGCCTCTCT
TCCCAGCTCACACATGAAGATGCACTTGCAAAGGGCTCTGGTGGTCCTGGCCCTGCTGAA
CTTTGCCACGGTCAGCCTCTCTCTGTCCACTTGCACCACCTTGGACTTCGGCCACATCAA
GAAGAAGAGGGTGGAAGCCATTAGGGGACAGATCTTGAGCAAGCTCAGGCTCACCAGCCC
CCCTGAGCCAACGGTGATGACCCACGTCCCCTATCAGGTCCTGGCCCTTTACAACAGCAC
CCGGGAGCTGCTGGAGGAGATGCATGGGGAGAGGGAGGAAGGCTGCACCCAGGAAAACAC
CGAGTCGGAATACTATGCCAAAGAAATCCATAAATTCGACATGATCCAGGGGCTGGCGGA
GCACAACGAACTGGCTGTCTGCCCTAAAGGAATTACCTCCAAGGTTTTCCGCTTCAATGT
GTCCTCAGTGGAGAAAAATAGAACCAACCTATTCCGAGCAGAATTCCGGGTCTTGCGGGT
GCCCAACCCCAGCTCTAAGCGGAATGAGCAGAGGATCGAGCTCTTCCAGATCCTTCGGCC
AGATGAGCACATTGCCAAACAGCGCTATATCGGTGGCAAGAATCTGCCCACACGGGGCAC
TGCCGAGTGGCTGTCCTTTGATGTCACTGACACTGTGCGTGAGTGGCTGTTGAGAAGAGA
GTCCAACTTAGGTCTAGAAATCAGCATTCACTGTCCATGTCACACCTTTCAGCCCAATGG
AGATATCCTGGAAAACATTCACGAGGTGATGGAAATCAAATTCAAAGGCGTGGACAATGA
GGATGACCATGGCCGTGGAGATCTGGGGCGCCTCAAGAAGCAGAAGGATCACCACAACCC
TCATCTAATCCTCATGATGATTCCCCCACACCGGCTCGACAACCCGGGCCAGGGGGGTCA
GAGGAAGAAGCGGGCTTTGGACACCAATTACTGCTTCCGCAACTTGGAGGAGAACTGCTG
TGTGCGCCCCCTCTACATTGACTTCCGACAGGATCTGGGCTGGAAGTGGGTCCATGAACC
TAAGGGCTACTATGCCAACTTCTGCTCAGGCCCTTGCCCATACCTCCGCAGTGCAGACAC
AACCCACAGCACGGTGCTGGGACTGTACAACACTCTGAACCCTGAAGCATCTGCCTCGCC
TTGCTGCGTGCCCCAGGACCTGGAGCCCCTGACCATCCTGTACTATGTTGGGAGGACCCC
CAAAGTGGAGCAGCTCTCCAACATGGTGGTGAAGTCTTGTAAATGTAGCTGAGACCCCAC
GTGCGACAGAGAGAGGGGAGAGAGAACCACCACTGCCTGACTGCCCGCTCCTCGGGAAAC
ACACAAGCAACAAACCTCACTGAGAGGCCTGGAGCCCACAACCTTCGGCTCCGGGCAAAT
GGCTGAGATGGAGGTTTCCTTTTGGAACATTTCTTTCTTGCTGGCTCTGAGAATCACGGT
GGTAAAGAAAGTGTGGGTTTGGTTAGAGGAAGGCTGAACTCTTCAGAACACACAGACTTT
CTGTGACGCAGACAGAGGGGATGGGGATAGAGGAAAGGGATGGTAAGTTGAGATGTTGTG
TGGCAATGGGATTTGGGCTACCCTAAAGGGAGAAGGAAGGGCAGAGAATGGCTGGGTCAG
GGCCAGACTGGAAGACACTTCAGATCTGAGGTTGGATTTGCTCATTGCTGTACCACATCT
GCTCTAGGGATCTGGATTATGTTATAcAGGCAAGCATTTTTTTTTTTTTAAAGACAGGTT
ACGAAGACAAAGTCCCAGAATTGTATCTCATACTGTCTGGGATTAAGGGCAAATCTATTA
CTTTTGCAAACTGTCCTCTACATCAATTAACATCGTGGGTCACTACAGGGAGAAAATCCA
GGTCATGCAGTTCCTGGCCCATCAACTGTATTGGGCCTTTTGGATATGCTGAACGCAGAA
GAAAGGGTGGAAATCAACCCTCTCCTGTCTGCCCTCTGGGTCCCTCCTCTCACCTCTCCC
TCGATCATATTTCCCCTTGGACACTTGGTTAGACGCCTTCCAGGTCAGGATGCACATTTC
TGGATTGTGGTTCCATGCAGCCTTGGGGCATTATGGGTCTTCCCCCACTTCCCCTCCAAG
ACCCTGTGTTCATTTGGTGTTCCTGGAAGCAGGTGCTACAACATGTGAGGCATTCGGGGA
AGCTGCACATGTGCCACACAGTGACTTGGCCCCAGACGCATAGACTGAGGTATAAAGACA
AGTATGAATATTACTCTCAAAATCTTTGTATAAATAAATATTTTTGGGGCATCCTGGATG
ATTTCATCTTCTGGAATATTGTTTCTAGAACAGTAAAAGCCTTATTCTAAGGTG
>gi|4507465|gb|NP_003230.1|TGFB3 412 aa linear transforming
growth factor, beta 3 [Homo sapiens].
MKMHLQRALVVLALLNFATVSLSLSTCTTLDFGHIKKKRVEAIRGQILSKLRLTSPPEPT
VMTHVPYQVLALYNSTRELLEEMHGEREEGCTQENTESEYYAKEIHKFDMIQGLAEHNEL
AVCPKGITSKVFRFNVSSVEKNRTNLFRAEFRVLRVPNPSSKRNEQRIELFQILRPDEHI
AKQRYIGGKNLPTRGTAEWLSFDVTDTVREWLLRRESNLGLEISIHCPCHTFQPNGDILE
NIHEVMEIKFKGVDNEDDHGRGDLGRLKKQKDHHNPHLILMMIPPHRLDNPGQGGQRKKR
ALDTNYCFRNLEENCCVRPLYIDFRQDLGWKWVHEPKGYYANFCSGPCPYLRSADTTHST
VLGLYNTLNPEASASPCCVPQDLEPLTILYYVGRTPKVEQLSNMVVKSCKCS
>gi|21735553|gb|NM_002419.2|MAP3K11 3603 bp mRNA Homo
sapiens mitogen-activated protein kinase kinase kinase 11
(MAP3K11), mRNA.
ACAAAGGGAGGAGGAAGAAGGGAGCGGGGTCGGAGCCGTCGGGGCCAAAGGAGACGGGGC
CAGGAACAGGCAGTCTCGGCCCAACTGCGGACGCTCCCTCCACCCCCTGCGCAAAAAGAC
CCAACCGGAGTTGAGGCGCTGCCCCTGAAGGCCCCACCTTACACTTGGCGGGGGCCGGAG
CCAGGCTCCCAGGACTGCTCCAGAACCGAGGGAAGCTCGGGTCCCTCCAAGCTAGCCATG
GTGAGGCGCCGGAGGCCCCGGGGCCCCACCCCCCCGGCCTGACCACACTGCCCTGGGTGC
CCTCCTCCAGAAGCCCGAGATGCGGGGGGCCGGGAGACAACACTCCTGGCTCCCCAGAGA
GGCGTGGGTCTGGGGCTGAGGGCCAGGGCCCGGATGCCCAGGTTCCGGGACTAGGGCCTT
GGCAGCCAGCGGGGGTGGGGACCACGGGCACCCAGAGAAGGTCCTCCACACATCCCAGCG
CCGGCTCCCGGCCATGGAGCCCTTGAAGAGCCTCTTCCTCAAGAGCCCTCTAGGGTCATG
GAATGGCAGTGGCAGCGGGGGTGGTGGGGGCGGTGGAGGAGGCCGGCCTGAGGGGTCTCC
AAAGGCAGCGGGTTATGCCAACCCGGTGTGGACAGCCCTGTTCGACTACGAGCCCAGTGG
GCAGGATGAGCTGGCCCTGAGGAAGGGTGACCGTGTGGAGGTGCTGTCCCGGGACGCAGC
CATCTCAGGAGACGAGGGCTGGTGGGCGGGCCAGGTGGGTGGCCAGGTGGGCATCTTCCC
GTCCAACTATGTGTCTCGGGGTGGTGGCCCGCCCCCCTGCGAGGTGGCCAGCTTCCAGGA
GCTGCGGCTGGAGGAGGTGATCGGCATTGGAGGCTTTGGCAAGGTGTACAGGGGCAGCTG
GCGAGGTGAGCTGGTGGCTGTGAAGGCAGCTCGCCAGGACCCCGATGAGGACATCAGTGT
GACAGCCGAGAGCGTTCGCCAGGAGGCCCGGCTCTTCGCCATGCTGGCACACCCCAACAT
CATTGCCCTCAAGGCTGTGTGCCTGGAGGAGCCCAACCTGTGCCTGGTGATGGAGTATGC
AGCCGGTGGGCCCCTCAGCCGAGCTCTGGCCGGGCGGCGCGTGCCTCCCCATGTGCTGGT
CAACTGGGCTGTGCAGATTGCCCGTGGGATGCACTACCTGCACTGCGAGGCCCTGGTGCC
CGTCATCCACCGTGATCTCAAGTCCAACAACATTTTGCTGCTGCAGCCCATTGAGAGTGA
CGACATGGAGCACAAGACCCTGAAGATCACCGACTTTGGCCTGGCCCGAGAGTGGCACAA
AACCACACAAATGAGTGCCGCGGGCACCTACGCCTGGATGGCTCCTGAGGTTATCAAGGC
CTCCACCTTCTCTAAGGGCAGTGACGTCTGGAGTTTTGGGGTGCTGCTGTGGGAACTGCT
GACCGGGGAGGTGCCATACCGTGGCATTGACTGCCTTGCTGTGGCCTATGGCGTAGCTGT
TAACAAGCTCACACTGCCCATCCCATCCACCTGCCCCGAGCCCTTCGCACAGCTTATGGC
CGACTGCTGGGCGCAGGACCCCCACCGCAGGCCCGACTTCGCCTCCATCCTGCAGCAGTT
GGAGGCGCTGGAGGCACAGGTCCTACGGGAAATGCCGCGGGACTCCTTCCATTCCATGCA
GGAAGGCTGGAAGCGCGAGATCCAGGGTCTCTTCGACGAGCTGCGAGCCAAGGAAAAGGA
ACTACTGAGCCGCGAGGAGGAGCTGACGCGAGCGGCGCGCGAGCAGCGGTCACAGGCGGA
GCAGCTGCGGCGGCGCGAGCACCTGCTGGCCCAGTGGGAGCTAGAGGTGTTCGAGCGCGA
GCTGACGCTGCTGCTGCAGCAGGTGGACCGCGAGCGACCGCACGTGCGCCGCCGCCGCGG
GACATTCAAGCGCAGCAAGCTCCGGGCGCGCGACGGCGGCGAGCGTATCAGCATGCCACT
CGACTTCAAGCACCGCATCACCGTGCAGGCCTCACCCGGCCTTGACCGGAGGAGAAACGT
CTTCGAGGTCGGGCCTGGGGATTCGCCCACCTTTCCCCGGTTCCGAGCCATCCAGTTGGA
GCCTGCAGAGCCAGGCCAGGCATGGGGCCGCCAGTCCCCCCGACGTCTGGAGGACTCAAG
CAATGGAGAGCGGCGAGCATGCTGGGCTTGGGGTCCCAGTTCCCCCAAGCCTGGGGAAGC
CCAGAATGGGAGGAGAAGGTCCCGCATGGACGAAGCCACATGGTACCTGGATTCAGATGA
CTCATCCCCCTTAGGATCTCCTTCCACACCCCCAGCACTCAATGGTAACCCCCCGCGGCC
TAGCCTGGAGCCCGAGGAGCCCAAGAGGCCTGTCCCCGCAGAGCGCGGTAGCAGCTCTGG
GACGCCCAAGCTGATCCAGCGGGCGCTGCTGCGCGGCACCGCCCTGCTCGCCTCGCTGGG
CCTTGGCCGCGACCTGCAGCCGCCGGGAGGCCCAGGACGCGAGCGCGGGGAGTCCCCGAC
AACACCCCCCACGCCAACGCCCGCGCCCTGCCCGACCGAGCCGCCCCCTTCCCCGCTCAT
CTGCTTCTCGCTCAAGACGCCCGACTCCCCGCCCACTCCTGCACCCCTGTTGCTGGACCT
GGGTATCCCTGTGGGCCAGCGGTCAGCCAAGAGCCCCCGACGTGAGGAGGAGCCCCGCGG
AGGCACTGTCTCACCCCCACCGGGGACATCACGCTCTGCTCCTGGCACCCCAGGCACCCC
ACGTTCACCACCCCTGGGCCTCATCAGCCGACCTCGGCCCTCGCCCCTTCGCAGCCGCAT
TGATCCCTGGAGCTTTGTGTCAGCTGGGCCACGGCCTTCTCCCCTGCCATCACCACAGCC
TGCACCCCGCCGAGCACCCTGGACCTTGTTCCCGGACTCAGACCCCTTCTGGGACTCCCC
ACCTGCCAACCCCTTCCAGGGGGGCCCCCAGGACTGCAGGGCACAGACCAAAGACATGGG
TGCCCAGGCCCCGTGGGTGCCGGAAGCGGGGCCTTGAGTGGGCCAGGCCACTCCCCCGAG
CTCCAGCTGCCTTAGGAGGAGTCACAGCATACACTGGAACAGGAGCTGGGTCAGCCTCTG
CAGCTGCCTCAGTTTCCCCAGGGACCCCACCCCCCTTTGGGGGTCAGGAACACTACACTG
CACAGGAAGCCTTCACACTGGAAGGGGGACCTGCGCCCCCACATCTGAAACCTGTAGGTC
CCCCCAGCTCACCTGCCCTACTGGGGCCCAACACTGTACCCAGCTGGTTGGGAGGACCAG
AGCCTGTCTCAGGGAATTGCCTGCTGGGGTGATGCAGGGAGGAGGGGAGGTGCAGGGAAG
AGGGGCCGGCCTCAGCTGTCACCAGCACTTTTGACCAAGTCCTGCTACTGCGGCCCCTGC
CCTAGGGCTTAGAGCATGGACCTCCTGCCCTGGGGGTCATCTGGGGCCAGGGCTCTCTGG
ATGCCTTCCTGCTGCCCCAGCCAGGGTTGGAGTCTTAGCCTCGGGATCCAGTGAAGCCAG
AAGCCAAATAAACTCAAAAGCTGTCTCCCCAAAAAAAAAAAAAAAAAAAAAAAAAAAAAA
AAA
>gi|4505195|gb|NP_002410.1|MAP3K11 847 aa linear mitogen-
activated protein kinase kinase kinase 11; mixed lineage
kinase 3; SH3 domain-containing proline-rich kinase;
protein-tyrosine kinase PTK1 [Homo sapiens].
MEPLKSLFLKSPLGSWNGSGSGGGGGGGGGRPEGSPKAAGYANPVWTALFDYEPSGQDEL
ALRKGDRVEVLSRDAAISGDEGWWAGQVGGQVGIFPSNYVSRGGGPPPCEVASFQELRLE
EVIGIGGFGKVYRGSWRGELVAVKAARQDPDEDISVTAESVRQEARLFAMLAHPNIIALK
AVCLEEPNLCLVMEYAAGGPLSRALAGRRVPPHVLVNWAVQIARGMHYLHCEALVPVIHR
DLKSNNILLLQPIESDDMEHKTLKITDFGLAREWHKTTQMSAAGTYAWMAPEVIKASTFS
KGSDVWSFGVLLWELLTGEVPYRGIDCLAVAYGVAVNKLTLPIPSTCPEPFAQLMADCWA
QDPHRRPDFASILQQLEALEAQVLREMPRDSFHSMQEGWKREIQGLFDELRAKEKELLSR
EEELTRAAREQRSQAEQLRRREHLLAQWELEVFERELTLLLQQVDRERPHVRRRRGTFKR
SKLRARDGGERISMPLDFKHRITVQASPGLDRRRNVFEVGPGDSPTFPRFPAIQLEPAEP
GQAWGRQSPRRLEDSSNGERRACWAWGPSSPKPGEAQNGRRRSRMDEATWYLDSDDSSPL
GSPSTPPALNGNPPRPSLEPEEPKRPVPAERGSSSGTPKLIQRALLRGTALLASLGLGRD
LQPPGGPGRERGESPTTPPTPTPAPCPTEPPPSPLICFSLKTPDSPPTPAPLLLDLGIPV
GQRSAKSPRREEEPRGGTVSPPPGTSRSAPGTPGTPRSPPLGLISRPRPSPLRSRIDPWS
FVSAGPRPSPLPSPQPAPRRAPWTLFPDSDPFWDSPPANPFQGGPQDCRAQTKDMGAQAP
WVPEAGP
>gi|4505784|gb|NM_000294.1|PHKG2 1571 bp mRNA Homo sapiens
phosphorylase kinase, gamma 2 (testis) (PHKG2), mRNA.
AAGGTGAGCGACTGCAGGCAAACCCGGCGACAGCGCAGCTCGCGTCGACCCTGGCTCCTC
TGCCTGCCCCCTCAGGCCCCCGCCTCCTTCAGGATGACGCTGGACGTGGGGCCGGAGGAT
GAGCTGCCCGACTGGGCCGCCGCCAAAGAGTTTTACCAGAAGTACGACCCTAAGGACGTC
ATCGGCAGAGGAGTGAGCTCTGTGGTCCGCCGTTGTGTTCATCGAGCTACTGGCCACGAG
TTTGCGGTGAAGATTATGGAAGTGACAGCTGAGCGGCTGAGTCCTGAGCAGCTGGAGGAG
GTGCGGGAAGCCACACGGCGAGAGACACACATCCTTCGCCAGGTCGCCGGCCACCCCCAC
ATCATCACCCTCATCGATTCCTACGAGTCTTCTAGCTTCATGTTCCTGGTGTTTGACCTG
ATGCGGAAGGGAGAGCTGTTTGACTATCTCACAGAGAAGGTGGCCCTCTCTGAAAAGGAA
ACCAGGTCCATCATGCGGTCTCTGCTGGAAGCAGTGAGCTTTCTCCATGCCAACAACATT
GTGCATCGAGATCTGAAGCCCGAGAATATTCTCCTAGATGACAATATGCAGATCCGACTT
TCAGATTTCGGGTTCTCCTGCCACTTGGAACCTGGCGAGAAGCTTCGAGAGTTGTGTGGG
ACCCCAGGGTATCTAGCGCCAGAGATCCTTAAATGCTCCATGGATGAAACCCACCCAGGC
TATGGCAAGGAGGTCGACCTCTGGGCCTGTGGGGTGATCTTGTTCACACTCCTGGCTGGC
TCGCCACCCTTCTGGCACCGGCGGCAGATCCTGATGTTACGCATGATCATGGAGGGCCAG
TACCAGTTCAGTTCCCCCGAGTGGGATGACCGTTCCAGCACTGTCAAAGACCTGATCTCC
AGGCTGCTGCAGGTGGATCCTGAGGCACGCCTGACAGCTGAGCAGGCCCTACAGCACCCC
TTCTTTGAGCGTTGTGAAGGCAGCCAACCCTGGAACCTCACCCCCCGCCAGCGGTTCCGG
GTGGCAGTGTGGACAGTGCTGGCTGCTGGACGAGTGGCCCTAAGCACCCATCGTGTACGG
CCACTGACCAAGAATGCACTGTTGAGGGACCCTTATGCGCTGCGGTCAGTGCGGCACCTC
ATCGACAACTGTGCCTTCCGGCTCTACGGGCACTGGGTAAAGAAAGGGGAGCAGCAGAAC
CGGGCGGCTCTCTTTCAGCACCGGCCCCCTGGGCCTTTTCCCATCATGGGCCCTGAAGAG
GAGGGAGACTCTGCTGCTATAACTGAGGATGAGGCCGTGCTTGTGCTGGGCTAGGACCTC
AACCCCAGGGATTCCCAGGAAGCAGAACTCTCCAGAAGAAGGGTTTTGATCATTCCAGCT
CCTCTGGGCTCTGGCCTCAGGCCCACTAATGATCCTGCTACCCTCTTGAAGACCAGCCCG
GTACCTCTCTCCCCACTGGCCAGGACTCTGAGATCAGAGCTGGGGTGGAAGGGAGCCATT
CTGAACGCCACGCCTGGCCCGGTCAGTGCTGCATGCACTGCATATGAAATAAAATCTGCT
ACACGCCAGGG
>gi|4505785|gb|NP_000285.1|PHKG2 406 aa linear phosphorylase
kinase, gamma 2 (testis); Phosphorylase kinase, gamma 2
(testis/liver) [Homo sapiens].
MTLDVGPEDELPDWAAAKEFYQKYDPKDVIGRGVSSVVRRCVHRATGHEFAVKIMEVTAE
RLSPEQLEEVREATRRETHILRQVAGHPHIITLIDSYESSSFMFLVFDLMRKGELFDYLT
EKVALSEKETRSIMRSLLEAVSFLHANNIVHRDLKPENILLDDNMQIRLSDFGFSCHLEP
GEKLRELCGTPGYLAPEILKCSMDETHPGYGKEVDLWACGVILFTLLAGSPPFWHRRQIL
MLRMIMEGQYQFSSPEWDDRSSTVKDLISRLLQVDPEARLTAEQALQHPFFERCEGSQPW
NLTPRQRFRVAVWTVLAAGRVALSTHRVRPLTKNALLRDPYALRSVRHLIDNCAFRLYGH
WVKKGEQQNRAALFQHRPPGPFPIMGPEEEGDSAAITEDEAVLVLG
>gi|5453789|gb|NM_006169.1|NNMT 952 bp mRNA Homo sapiens
nicotinamide N-methyltransferase (NNMT), mRNA.
TGAACTCTGGATGCTGTTAGCCTGAGACTCAGGAAGACAACTTCTGCAGGGTCACTCCCT
GGCTTCTGGAGGAAAGAGAAGGAGGGCAGTGCTCCAGTGGTACAGAAGTGAGACATAATG
GAATCAGGCTTCACCTCCAAGGACACCTATCTAAGCCATTTTAACCCTCGGGATTACCTA
GAAAAATATTACAAGTTTGGTTCTAGGCACTCTGCAGAAAGCCAGATTCTTAAGCACCTT
CTGAAAAATCTTTTCAAGATATTCTGCCTAGACGGTGTGAAGGGAGACCTGCTGATTGAC
ATCGGCTCTGGCCCCACTATCTATCAGCTCCTCTCTGCTTGTGAATCCTTTAAGGAGATC
GTCGTCACTGACTACTCAGACCAGAACCTGCAGGAGCTGGAGAAGTGGCTGAAGAAAGAG
CCAGAGGCCTTTGACTGGTCCCCAGTGGTGACCTATGTGTGTGATCTTGAAGGGAACAGA
GTCAAGGGTCCAGAGAAGGAGGAGAAGTTGAGACAGGCGGTCAAGCAGGTGCTGAAGTGT
GATGTGACTCAGAGCCAGCCACTGGGGGCCGTCCCCTTACCCCCGGCTGACTGCGTGCTC
AGCACACTGTGTCTGGATGCCGCCTGCCCAGACCTCCCCACCTACTGCAGGGCGCTCAGG
AACCTCGGCAGCCTACTGAAGCCAGGGGGCTTCCTGGTGATCATGGATGCGCTCAAGAGC
AGCTACTACATGATTGGTGAGCAGAAGTTCTCCAGCCTCCCCCTGGGCCGGGAGGCAGTA
GAGGCTGCTGTGAAAGAGGCTGGCTACACAATCGAATGGTTTGAGGTGATCTCGCAAAGT
TATTCTTCCACCATGGCCAACAACGAAGGACTTTTCTCCCTGGTGGCGAGGAAGCTGAGC
AGACCCCTGTGATGCCTGTGACCTCAATTAAAGCAATTCCTTTGACCTGTCA
>gi|5453790|gb|NP_006160.1|NNMT 264 aa linear nicotinamide
N-methyltransferase [Homo sapiens].
MESGFTSKDTYLSHFNPRDYLEKYYKFGSRHSAESQILKHLLKNLFKIFCLDGVKGDLLI
DIGSGPTIYQLLSACESFKEIVVTDYSDQNLQELEKWLKKEPEAFDWSPVVTYVCDLEGN
RVKGPEKEEKLRQAVKQVLKCDVTQSQPLGAVPLPPADCVLSTLCLDAACPDLPTYCRAL
RNLGSLLKPGGFLVIMDALKSSYYMIGEQKFSSLPLGREAVEAAVKEAGYTIEWFEVISQ
SYSSTMANNEGLFSLVARKLSRPL
>gi|4507668|gb|NM_003295.1|TPT1 830 bp mRNA Homo sapiens
tumor protein, translationally-controlled 1 (TPT1), mRNA.
CCCCCCCGAGCGCCGCTCCGGCTGCACCGCGCTCGCTCCGAGTTTCAGGCTCGTGCTAAG
CTAGCGCCGTCGTCGTCTCCCTTCAGTCGCCATCATGATTATCTACCGGGACCTCATCAG
CCACGATGAGATGTTCTCCGACATCTACAAGATCCGGGAGATCGCGGACGGGTTGTGCCT
GGAGGTGGAGGGGAAGATGGTCAGTAGGACAGAAGGTAACATTGATGACTCGCTCATTGG
TGGAAATGCCTCCGCTGAAGGCCCCGAGGGCGAAGGTACCGAAAGCACAGTAATCACTGG
TGTCGATATTGTCATGAACCATCACCTGCAGGAAACAAGTTTCACAAAAGAAGCCTACAA
GAAGTACATCAAAGATTACATGAAATCAATCAAAGGGAAACTTGAAGAACAGAGACCAGA
AAGAGTAAAACCTTTTATGACAGGGGCTGCAGAACAAATCAAGCACATCCTTGCTAATTT
CAAAAACTACCAGTTCTTTATTGGTGAAAACATGAATCCAGATGGCATGGTTGCTCTATT
GGACTACCGTGAGGATGGTGTGACCCCATATATGATTTTCTTTAAGGATGGTTTAGAAAT
GGAAAAATGTTAACAAATGTGGCAATTATTTTGGATCTATCACCTGTCATCATAACTGGC
TTCTGCTTGTCATCCACACAACACCAGGACTTAAGACAAATGGGACTGATGTCATCTTGA
GCTCTTCATTTATTTTGACTGTGATTTATTTGGAGTGGAGGCATTGTTTTTAAGAAAAAC
ATGTCATGTAGGTTGTCTAAAAATAAAATGCATTTAAACTCATTTGAGAG
>gi|4507669|gb|NP_003286.1|TPT1 172 aa linear tumor protein,
translationally-controlled 1; fortilin; histamine-releasing
factor [Homo sapiens].
MIIYRDLISHDEMFSDIYKIREIADGLCLEVEGKMVSRTEGNIDDSLIGGNASAEGPEGE
GTESTVITGVDIVMNHHLQETSFTKEAYKKYIKDYMKSIKGKLEEQRPERVKPFMTGAAE
QIKHILANFKNYQFFIGENMNPDGMVALLDYREDGVTPYMIFFKDGLEMEKC
>gi|27477073|gb|NM_018725.2|IL17BR 2077 bp mRNA Homo sapiens
interleukin 17B receptor (IL17BR), transcript variant 1,
mRNA.
AGCGCAGCGTGCGGGTGGCCTGGATCCCGCGCAGTGGCCCGGCGATGTCGCTCGTGCTGC
TAAGCCTGGCCGCGCTGTGCAGGAGCGCCGTACCCCGAGAGCCGACCGTTCAATGTGGCT
CTGAAACTGGGCCATCTCCAGAGTGGATGCTACAACATGATCTAATCCCCGGAGACTTGA
GGGACCTCCGAGTAGAACCTGTTACAACTAGTGTTGCAACAGGGGACTATTCAATTTTGA
TGAATGTAAGCTGGGTACTCCGGGCAGATGCCAGCATCCGCTTGTTGAAGGCCACCAAGA
TTTGTGTGACGGGCAAAAGCAACTTCCAGTCCTACAGCTGTGTGAGGTGCAATTACACAG
AGGCCTTCCAGACTCAGACCAGACCCTCTGGTGGTAAATGGACATTTTCCTACATCGGCT
TCCCTGTAGAGCTGAACACAGTCTATTTCATTGGGGCCCATAATATTCCTAATGCAAATA
TGAATGAAGATGGCCCTTCCATGTCTGTGAATTTCACCTCACCAGGCTGCCTAGACCACA
TAATGAAATATAAAAAAAAGTGTGTCAAGGCCGGAAGCCTGTGGGATCCGAACATCACTG
CTTGTAAGAAGAATGAGGAGACAGTAGAAGTGAACTTCACAACCACTCCCCTGGGAAACA
GATACATGGCTCTTATCCAACACAGCACTATCATCGGGTTTTCTCAGGTGTTTGAGCCAC
ACCAGAAGAAACAAACGCGAGCTTCAGTGGTGATTCCAGTGACTGGGGATAGTGAAGGTG
CTACGGTGCAGCTGACTCCATATTTTCCTACTTGTGGCAGCGACTGCATCCGACATAAAG
GAACAGTTGTGCTCTGCCCACAAACAGGCGTCCCTTTCCCTCTGGATAACAACAAAAGCA
AGCCGGGAGGCTGGCTGCCTCTCCTCCTGCTGTCTCTGCTGGTGGCCACATGGGTGCTGG
TGGCAGGGATCTATCTAATGTGGAGGCACGAAAGGATCAAGAAGACTTCCTTTTCTACCA
CCACACTACTGCCCCCCATTAAGGTTCTTGTGGTTTACCCATCTGAAATATGTTTCCATC
ACACAATTTGTTACTTCACTGAATTTCTTCAAAACCATTGCAGAAGTGAGGTCATCCTTG
AAAAGTGGCAGAAAAAGAAAATAGCAGAGATGGGTCCAGTGCAGTGGCTTGCCACTCAAA
AGAAGGCAGCAGACAAAGTCGTCTTCCTTCTTTCCAATGACGTCAACAGTGTGTGCGATG
GTACCTGTGGCAAGAGCGAGGGCAGTCCCAGTGAGAACTCTCAAGACCTCTTCCCCCTTG
CCTTTAACCTTTTCTGCAGTGATCTAAGAAGCCAGATTCATCTGCACAAATACGTGGTGG
TCTACTTTAGAGAGATTGATACAAAAGACGATTACAATGCTCTCAGTGTCTGCCCCAAGT
ACCACCTCATGAAGGATGCCACTGCTTTCTGTGCAGAACTTCTCCATGTCAAGCAGCAGG
TGTCAGCAGGAAAAAGATCACAAGCCTGCCACGATGGCTGCTGCTCCTTGTAGCCCACCC
ATGAGAAGCAAGAGACCTTAAAGGCTTCCTATCCCACCAATTACAGGGAAAAAACGTGTG
ATGATCCTGAAGCTTACTATGCAGCCTACAAACAGCCTTAGTAATTAAAACATTTTATAC
CAATAAAATTTTCAAATATTGCTAACTAATGTAGCATTAACTAACGATTGGAAACTACAT
TTACAACTTCAAAGCTGTTTTATACATAGAAATCAATTACAGTTTTAATTGAAAACTATA
ACCATTTTGATAATGCAACAATAAAGCATCTTCAGCCAAACATCTAGTCTTCCATAGACC
ATGCATTGCAGTGTACCCAGAACTGTTTAGCTAATATTCTATGTTTAATTAATGAATACT
AACTCTAAGAACCCCTCACTGATTCACTCAATAGCATCTTAAGTGAAAAACCTTCTATTA
CATGCAAAAAATCATTGTTTTTAAGATAACAAAAGTAGGGAATAAACAAGCTGAACCCAC
TTTTAAAAAAAAAAAAAAAAAAAAAAAAAAAAAAAAA
>gi|27477074|gb|NP_061195.2|IL17BR 502 aa linear IL-17B
receptor isoform 1 precursor; IL-17B receptor; interleukin
17 receptor homolog 1; interleukin 17 receptor homolog;
cytokine receptor CRL4 [Homo sapiens].
MSLVLLSLAALCRSAVPREPTVQCGSETGPSPEWMLQHDLIPGDLRDLRVEPVTTSVATG
DYSILMNVSWVLRADASIRLLKATKICVTGKSNFQSYSCVRCNYTEAFQTQTRPSGGKWT
FSYIGFPVELNTVYFIGAHNIPNANMNEDGPSMSVNFTSPGCLDHIMKYKKKCVKAGSLW
DPNITACKKNEETVEVNFTTTPLGNRYMALIQHSTIIGFSQVFEPHQKKQTRASVVIPVT
GDSEGATVQLTPYFPTCGSDCIRHKGTVVLCPQTGVPFPLDNNKSKPGGWLPLLLLSLLV
ATWVLVAGIYLMWRHERIKKTSFSTTTLLPPIKVLVVYPSEICFHHTICYFTEFLQNHCR
SEVILEKWQKKKIAEMGPVQWLATQKKAADKVVFLLSNDVNSVCDGTCGKSEGSPSENSQ
DLFPLAFNLFCSDLRSQIHLHKYVVVYFREIDTKDDYNALSVCPKYHLMKDATAFCAELL
HVKQQVSAGKRSQACHDGCCSL
>gi|14165275|gb|NM_032411.1|ECRG4 772 bp mRNA Homo sapiens
esophageal cancer related gene 4 protein (ECRG4), mRNA.
GGATAACCCGCGGCCGCGCCTGCCCGCTCGCACCCCTCTCCCGCGCCCGGTTCTCCCTCG
CAGCACCTCGAAGTGCGCCCCTCGCCCTCCTGCTCGCGCCCCGCCGCCATGGCTGCCTCC
CCCGCGCGGCCTGCTGTCCTGGCCCTGACCGGGCTGGCGCTGCTCCTGCTCCTGTGCTGG
GGCCCAGGTGGCATAAGTGGAAATAAACTCAAGCTGATGCTTCAAAAACGAGAAGCACCT
GTTCCAACTAAGACTAAAGTGGCCGTTGATGAGAATAAAGCCAAAGAATTCCTTGGCAGC
CTGAAGCGCCAGAAGCGGCAGCTGTGGGACCGGACTCGGCCCGAGGTGCAGCAGTGGTAC
CAGCAGTTTCTCTACATGGGCTTTGACGAAGCGAAATTTGAAGATGACATCACCTATTGG
CTTAACAGAGATCGAAATGGACATGAATACTATGGCGATTACTACCAACGTCACTATGAT
GAAGACTCTGCAATTGGTCCCCGGAGCCCCTACGGCTTTAGGCATGGAGCCAGCGTCAAC
TACGATGACTACTAACCATGACTTGCCACACGCTGTACAAGAAGCAAATAGCGATTCTCT
TCATGTATCTCCTAATGCCTTACACTACTTGGTTTCTGATTTGCTCTATTTCAGCAGATC
TTTCTACCTACTTTGGTGATCAAAAAAGAAGAGTTAAAACAACACATGTAAATGCCTTTT
GATATTTCATGGGAATGTTTAAAAATAGAAATAAAGCATTTTGTTAAAACGA
>gi|14165276|gb|NP_115787.1|ECRG4 148 aa linear esophageal
cancer related gene 4 protein [Homo sapiens].
MAASPARPAVLALTGLALLLLLCWGPGGISGNKLKLMLQKREAPVPTKTKVAVDENKAKE
FLGSLKRQKRQLWDRTRPEVQQWYQQFLYMGFDEAKFEDDITYWLNRDRNGHEYYGDYYQ
RHYDEDSAIGPRSPYGFRHGASVNYDDY

1. A method for identifying a nucleic acid associated with osteoarthritis (OA), which method comprises:
(a) transfecting a cell with a nucleic acid so that the nucleic acid is expressed by the cell; and
(b) detecting expression by the cell of one or more marker nucleic acids, each of said one or more marker nucleic acids being associated with OA
wherein expression of the one or more marker nucleic acids by the cell identifies the nucleic acid transfected into the cell as a nucleic acid associated with OA.

2. A method according to claim 1 wherein the cell is a chondrocyte cell.

3. A method according to claim 1 wherein the cell is a human chondrocyte cell.

4. A method according to claim 1 wherein at least one of the one or more marker nucleic acids is selected from the group consisting of: Aggrecanase-1, MMP-13, Collagen Type I, Collagen Type IIa, Collagen Type X, iNOS, Cox-2, Aggrecan and Decorin.

5. A method according to claim 1 wherein at least one of the one or more marker nucleic acids is selected from the group consisting of C17, SMOC2, OSF-2, MARCKS, retinoic acid receptor beta, Zic1, BASP1 and DIM1.

6. A method according to claim 1 in which expression of the one or more marker nucleic acids is detected by RT-PCR.

7. A method for identifying a nucleic acid associated with osteoarthritis (OA), which method comprises:
(a) transfecting a cell with a nucleic acid so that the nucleic acid is expressed by the cell; and
(b) detecting expression by the cell of one or more marker polypeptides, each of said one or more marker nucleic acids being associated with OA
wherein expression of the one or more marker polypeptides by the cell identifies the nucleic acid transfected into the cell as a nucleic acid associated with OA.

8. A method according to claim 7 wherein the cell is a chondrocyte cell.

9. A method according to claim 7 wherein the cell is a human chondrocyte cell.

10. A method according to claim 7 wherein the marker polypeptide is selected from the group consisting of: Aggrecanase-1, MMP-13, Collagen Type I, Collagen Type IIa, Collagen Type X, iNOS, Cox-2, Aggrecan and Decorin.

11. A method for identifying a polypeptide associated with osteoarthritis (OA), which method comprises:
(a) transfecting a cell with a nucleic acid that encodes a polypeptide, so that the polypeptide is expressed by the cell; and
(b) detecting expression by the cell of one or more marker nucleic acids, each of said one or more marker nucleic acids being associated with OA,
wherein expression of the one or more marker nucleic acids identifies the polypeptide expressed by the nucleic acid transfected into the cell as a polypeptide that is associated with OA.

12. A method according to claim 11 wherein the cell is a chondrocyte cell.

13. A method according to claim 11 wherein the cell is a human chondrocyte cell.

14. A method according to claim 11 wherein at least one of the marker nucleic acids is selected from the group consisting of: Aggrecanase-1, MMP-1 3, Collagen Type I, Collagen Type Ila, Collagen Type X, iNOS, Cox-2, Aggrecan and Decorin.

15. A method according to claim 11 wherein at least one of the one or more marker nucleic acids is selected from the group consisting of C17, SMOC2, OSF-2, MARCKS, retinoic acid receptor beta, Zic1, BASP 1 and DIM1.

16. A method according to claim 11 wherein expression of the marker nucleic acids is detected by RT-PCR.

17. A method for identifying a polypeptide associated with osteoarthritis (OA), which method comprises:
(a) transfecting a cell with a nucleic acid that encodes a polypeptide, so that the polypeptide is expressed by the cell; and
(b) detecting expression by the cell of one or more marker polypeptides, each of said one or more marker nucleic acids being associated with OA.
wherein expression of the one or more marker polypeptides by the cell identifies the polypeptide expressed by the nucleic acid as a polypeptide that is associated with OA.

18. A method according to claim 17 wherein the cell is a chondrocyte cell.

19. A method according to claim 17 wherein the cell is a human chondrocyte cell.

20. A method according to claim 17 wherein the marker polypeptide is selected from the group consisting of: Aggrecanase-1, MMP-1 3, Collagen Type I, Collagen Type Ila, Collagen Type X, iNOS, Cox-2, Aggrecan and Decorin.

21. A method for identifying a nucleic acid associated with osteoarthritis (OA), which method comprises:
(a) transfecting a chondrocyte cell with a nucleic acid so that the nucleic acid is expressed by the chondrocyte cell; and
(b) detecting proliferation of the chondrocyte cell
wherein proliferation of the chondrocyte cell indicates that the nucleic acid is associated with OA.

22. A method according to claim 21 in which the chondrocyte cell is a human chondrocyte cell.

23. A method according to claim 21 wherein proliferation of the chondrocyte cell is detected by a method which comprises:
(i) culturing the chondrocyte cell, and
(ii) identifying clusters of chondrocyte cells in said cell culture, said clusters being indicative of cells proliferation.

24. A method for identifying a polypeptide associated with osteoarthritis (OA), which method comprises:
(a) transfecting a chondrocyte cell with a nucleic acid that encodes a polypeptide, so that the polypeptide is expressed by the chondrocyte cell; and
(b) detecting proliferation of the chondrocyte cell,
wherein proliferation of the chondrocyte cell identifies the nucleic acid as a nucleic acid associated with OA.

25. A method according to claim 24 in which the chondrocyte cell is a human chondrocyte cell.

26. A method according to claim 24 wherein proliferation of the chondrocyte cell is detected by a method which comprises:
(i) culturing the chondrocyte cell, and
(ii) identifying clusters of chondrocyte cells in said cell culture, said clusters being indicative of cells proliferation.

27. A method for identifying an individual having osteoarthritis (OA), which method comprises:
(a) detecting a candidate gene or gene product in cartilage or chondrocyte cells from an individual, said candidate gene or gene product being a gene or gene product set forth in Table V or VI; and
(b) comparing the level of said candidate gene or gene product in the individual to levels of the candidate gene or gene product in individuals not having osteoarthritis,
wherein elevated levels of the candidate gene or gene product in cartilage or chondrocytes derived from the individual indicates that the individual has OA.

28. A method for identifying a compound that may be used to treat, prevent or ameliorate osteoarthritis (OA), which method comprises:
(a) contacting a test compound to a cell;
(b) detecting expression by the cell of a candidate gene or gene product, said candidate gene or gene product being a gene or gene product set forth in Table V or VI; and
(c) comparing the level of the candidate gene or gene product expressed by the cell contacted with the test compound to the level of expression by a cell that is not contacted with the test compound,
wherein a decreased expression of the candidate gene or gene product by the cell contacted with the test compound indicates that the test compound may be used to treat OA.

29. A method according to claim 28 wherein the cell is a chondrocyte cell

30. A method according to claim 28 wherein the cell is a human chondrocyte cell.

31. A method to treat, prevent or ameliorate OA comprising administering to a subject in need thereof an effective amount of one or more modulators of one or more candidate genes selected from the group consisting of those disclosed in Table V and Table VI.

32. The method of claim 31 wherein said modulator inhibits activity of the gene product encoded by said candidate gene in said subject.

33. The method of claim 31 wherein said modulator inhibits the expression of said candidate gene in said subject.

34. The method of claim 31 wherein said modulator comprises any one or more substances selected from the group consisting of antisense oligonucleotides, triple helix DNA, ribozymes, RNA aptamers, siRNA and double or single stranded RNA wherein said substances are designed to inhibit the expression of said candidate gene.

35. The method of claim 31 wherein said modulator comprises one or more antibodies to a gene product or fragments thereof, encoded by said candidate gene wherein said antibodies or fragments thereof can inhibit activity of said gene product.

36. A method to treat, prevent or ameliorate OA comprising administering to a subject in need thereof a pharmaceutical composition comprising an effective amount of one or more modulators of any one or more candidate genes selected from the group consisting of those disclosed in Table V and Table VI.

37. The method of claim 36 wherein said modulator inhibits activity of the gene product encoded by said candidate gene in said subject.

38. The method of claim 36 wherein said modulator inhibits the expression of said candidate gene in said subject.

39. The method of claim 36 wherein said modulator comprises any one or more substances selected from the group consisting of antisense oligonucleotides, triple helix DNA, ribozymes, RNA aptamers, si RNA and double or single stranded RNA wherein said substances are designed to inhibit expression of said candidate gene.

40. The method of claim 36 wherein said modulator comprises one or more antibodies to a gene product or fragments thereof, encoded by said candidate gene wherein said antibodies or fragments thereof can inhibit activity of said gene product.

41. A pharmaceutical composition comprising one or more modulators of any one or more candidate genes selected from the group consisting of those disclosed in Table V and Table VI in an amount effective to treat or ameliorate OA in a subject in need thereof.

42. The pharmaceutical composition according to claim 41 wherein said modulator inhibits the activity of the gene product encoded by said candidate gene in said subject.

43. The pharmaceutical composition of claim 41 wherein said modulator inhibits the expression of said candidate gene in said subject.

44. The pharmaceutical composition of claim 41 wherein said modulator comprises any one or more substances selected from the group consisting of antisense oligonucleotides, triple helix DNA, ribozymes, RNA aptamers, siRNA and double or single stranded RNA wherein said substances are designed to inhibit the expression of said candidate gene.

45. The pharmaceutical composition of claim 41 wherein said modulator comprises one or more antibodies to a gene product or fragments thereof, encoded by said candidate gene wherein said antibodies or fragments thereof can inhibit activity of said gene product.

46. A method to treat, prevent or ameliorate OA comprising
(a) assaying a subject for mRNA levels for any one or more candidate genes selected from the group consisting of those disclosed in Table V and Table VI.; and
(b) administering to a subject with increased levels of mRNA compared to controls a modulator of any one or more of said candidate genes in an amount sufficient to treat, prevent or ameliorate OA.

47. A method to treat, prevent or ameliorate OA comprising:
(a) assaying a subject for levels of any one or more gene products encoded by a candidate gene selected from the group consisting of those disclosed in Table V and Table VI; and,
(b) administering to a subject with increased levels compared to controls a modulator of any one or more of said gene products in an amount sufficient to treat, prevent or ameliorate OA.

48. A diagnostic kit for detecting mRNA levels or protein levels of a candidate gene or gene product selected from the group consisting of those disclosed in Table V and Table VI, said kit comprising:
(a) a polynucleotide of said candidate gene or a fragment thereof;
(b) a nucleotide sequence complementary to that of (a);
(c) an expression product of said candidate gene, or a fragment thereof; or
(d) an antibody to said expression product
wherein components (a), (b), (c) or (d) may comprise a substantial component.


SEQUENCE LISTING (180 sequences)

SEQ ID NO: 1 - DNA, 21 bp, unknown
  primer sequence which can bind to any organism
tttccctggcaaggactatga

SEQ ID NO: 2 - DNA, 17 bp, unknown
  primer sequence which can bind to any organism
aatggcgtgagtcgggc

SEQ ID NO: 3 - DNA, 26 bp, unknown
  primer sequence which can bind to any organism
tgatctcttttggaattaaggagcat

SEQ ID NO: 4 - DNA, 23 bp, unknown
  primer sequence which can bind to any organism
atgggcatctcctccataatttg

SEQ ID NO: 5 - DNA, 19 bp, unknown
  primer sequence which can bind to any organism
aaattgctggcagggttgc

SEQ ID NO: 6 - DNA, 21 bp, unknown
  primer sequence which can bind to any organism
tttctgtactgcgggtggaac

SEQ ID NO: 7 - DNA, 19 bp, unknown
  primer sequence which can bind to any organism
gcaaaccttcaaggcagcc

SEQ ID NO: 8 - DNA, 19 bp, unknown
  primer sequence which can bind to any organism
tgctgtttgcctcggacat

SEQ ID NO: 9 - DNA, 16 bp, unknown
  primer sequence which can bind to any organism
acgctgctcgtcgccg

SEQ ID NO: 10 - DNA, 20 bp, unknown
  primer sequence which can bind to any organism
gccagcctcctggacatcct

SEQ ID NO: 11 - DNA, 23 bp, unknown
  primer sequence which can bind to any organism
acccaacaccaagacacagttct

SEQ ID NO: 12 - DNA, 33 bp, unknown
  primer sequence which can bind to any organism
tcttactgctatacctttactctttatggtgta

SEQ ID NO: 13 - DNA, 19 bp, unknown
  primer sequence which can bind to any organism
cagccgcttcacctacagc

SEQ ID NO: 14 - DNA, 25 bp, unknown
  primer sequence which can bind to any organism
ttttgtattcaatcactgtcttgcc

SEQ ID NO: 15 - DNA, 20 bp, unknown
  primer sequence which can bind to any organism
gccagcctcctggacatcct

SEQ ID NO: 16 - DNA, 23 bp, unknown
  primer sequence which can bind to any organism
agtcctttcaggctagctgcatc

SEQ ID NO: 17 - DNA, 17 bp, unknown
  primer sequence which can bind to any organism
tcgaggacagcgaggcc

SEQ ID NO: 18 - DNA, 22 bp, unknown
  primer sequence which can bind to any organism
tcgagggtgtagcgtgtagaga

SEQ ID NO: 19 - DNA, 19 bp, unknown
  primer sequence which can bind to any organism
atggggaaggtgaaggtcg

SEQ ID NO: 20 - DNA, 20 bp, unknown
  primer sequence which can bind to any organism
taaaagcagccctggtgacc

SEQ ID NO: 21 - DNA, 19 bp, unknown
  primer sequence which can bind to any organism
caagtttgtacaaaaaagc

SEQ ID NO: 22 - DNA, 19 bp, unknown
  primer sequence which can bind to any organism
accactttgtacaagaaag

SEQ ID NO: 23 - protein, 836 aa, homo sapiens
MIPFLPMFSLLLLLIVNPINANNHYDKILAHSRIRGRDQGPNVCALQQILGTKKKYFSTCKNWYKKSICGQKTTVLYECCPGYMRMEGMKGCPAVLPIDHVYGTLGIVGATTTQRYSDASKLREEIEGKGSFTYFAPSNEAWDNLDSDIRRGLESNVNVELLNALHSHMINKRMLTKDLKNGMIIPSMYNNLGLFINHYPNGVVTVNCARIIHGNQIATNGVVHVIDRVLTQIGTSIQDFIEAEDDLSSFRAAAITSDILEALGRDGHFTLFAPTNEAFEKLPRGVLERFMGDKVASEALMKYHILNTLQCSESIMGGAVFETLEGNTIEIGCDGDSITVNGIKMVNKKDIVTNNGVIHLIDQVLIPDSAKQVIELAGKQQTTFTDLVAQLGLASALRPDGEYTLLAPVNNAFSDDTLSMVQRLLKLILQNHILKVKVGLNELYNGQILETIGGKQLRVFVYRTAVCIENSCMEKGSKQGRNGAIHIFREIIKPAEKSLHEKLKQDKRFSTFLSLLEAADLKELLTQPGDWTLFVPTNDAFKGMTSEEKEILIRDKNALQNIILYHLTPGVFIGKGFEPGVTNILKTTQGSKIFLKEVNDTLLVNELKSKESDIMTTNGVIHVVDKLLYPADTPVGNDQLLEILNKLIKYIQIKFVRGSTFKEIPVTVYTTKIITKVVEPKIKVIEGSLQPIIKTEGPTLTKVKIEGEPEFRLIKEGETITEVIHGEPIIKKYTKIIDGVPVEITEKETREERIITGPEIKYTRISTGGGETEETLKKLLQEEVTKVTKFIEGGDGHLFEDEEIKRLLQGDTPVRKLQANKKVQGSRRRLREGRSQ

SEQ ID NO: 24 - protein, 332 aa, homo sapiens
MGAQFSKTAAKGEAAAERPGEAAVASSPSKANGQENGHVKVNGDASPAAAESGAKEELQANGSAPAADKEEPAAAGSGAASPSSAEKGEPAAAAAPEAGASPVEKEAPAEGEAAEPGSATAAEGEAASAASSTSSPKAEDGATPSPSNETPKKKKKRFSFKKSFKLSGFSFKKNKKEAGEGGEAEAPAAEGGKDEAAGGAAAAAAEAGAASGEQAAAPGEEAAAGEEGAAGGDPQEAKPQEAAVAPEKPPASDETKAAEEPSKVEEKKAEEAGASAAACEAPSAAGPGAPPEQEAAPAEEPAAAAASSACAAPSQEAQPECSPEAPPAEAAE

SEQ ID NO: 25 - protein, 448 aa, homo sapiens
MFDCMDVLSVSPGQILDFYTASPSSCMLQEKALKACFSGLTQTEWQHRHTAQSIETQSTSSEELVPSPPSPLPPPRVYKPCFVCQDKSSGYHYGVSACEGCKGFFRRSIQKNMIYTCHRDKNCVINKVTRNRCQYCRLQKCFEVGMSKESVRNDRNKKKKETSKQECTESYEMTAELDDLTEKIRKAHQETFPSLCQLGKYTTNSSADHRVRLDLGLWDKFSELATKCIIKIVEFAKRLPGFTGLTIADQITLLKAACLDILILRICTRYTPEQDTMTFSDGLTLNRTQMHNAGFGPLTDLVFTFANQLLPLEMDDTETGLLSAICLICGDRQDLEEPTKVDKLQEPLLEALKIYIRKRRPSKPHMFPKILMKITDLRSISAKGAERVITLKMEIPGSMPPLIQEMLENSEGHEPLTPSSSGNTAEHSPSISPSSVENSGVSQSPLVQ

SEQ ID NO: 26 - protein, 336 aa, homo sapiens
MIYTCHRDKNCVINKVTRNRCQYCRLQKCFEVGMSKESVRNDRNKKKKETSKQECTESYEMTAELDDLTEKIRKAHQETFPSLCQLGKYTTNSSADHRVRLDLGLWDKFSELATKCIIKIVEFAKRLPGFTGLTIADQITLLKAACLDILILRICTRYTPEQDTMTFSDGLTLNRTQMHNAGFGPLTDLVFTFANQLLPLEMDDTETGLLSAICLICGDRQDLEEPTKVDKLQEPLLEALKIYIRKRRPSKPHMFPKILMKITDLRSISAKGAERVITLKMEIPGSMPPLIQEMLENSEGHEPLTPSSSGNTAEHSPSISPSSVENSGVSQSPLVQ

SEQ ID NO: 27 - protein, 227 aa, homo sapiens
MGGKLSKKKKGYNVNDEKAKEKDKKAEGAATEEEGTPKESEPQAAAEPAEAKEGKEKPDQDAEGKAEEKEGEKDAAAAKEEAPKAEPEKTEGAAEAKAEPPKAPEQEQAAPGPAAGGEAPKAAEAAAAPAESAAPAAGEEPSKEEGEPKKTEAPAAPAAQETKSDGAPASDSKPGSSEAAPSSKETPAATEAPSSTPKAQGPAASAEEPKPVEAPAANSDQTVTVKE

SEQ ID NO: 28 - protein, 447 aa, homo sapiens
MLLDAGPQYPAIGVTTFGASRHHSAGDVAERDVGLGINPFADGMGAFKLNPSSHELASAGQTAFTSQAPGYAAAAALGHHHHPGHVGSYSSAAFNSTRDFLFRNRGFGDAAAAASAQHSLFAASAGGFGGPHGHTDAAGHLLFPGLHEQAAGHASPNVVNGQMRLGFSGDMYPRPEQYGQVTSPRSEHYAAPQLHGYGPMNVNMAAHHGAGAFFRYMRQPIKQELICKWIEPEQLANPKKSCNKTFSTMHELVTHVTVEHVGGPEQSNHICFWEECPREGKPFKAKYKLVNHIRVHTGEKPFPCPFPGCGKVFARSENLKIHKRTHTGEKPFKCEFEGCDRRFANSSDRKKHMHVHTSDKPYLCKMCDKSYTHPSSLRKHMKVHESSSQGSQPSPAASSGYESSTPPTIVSPSTDNPTTSSLSPSSSAVHHTAGHSALSSNFNEWYV

SEQ ID NO: 29 - protein, 142 aa, homo sapiens
MSYMLPHLHNGWQVDQAILSEEDRVVVIRFGHDWDPTCMKMDEVLYSIAEKVKNFAVIYLVDITEVPDFNKMYELYDPCTVMFFFRNKHIMIDLGTGNNNKINWAMEDKQEMVDIIETVYRGARKGRGLVVSPKDYSTKYRY

SEQ ID NO: 30 - DNA, 1403 bp, homo sapiens
ccgggtgagtgagagagttggttggtgttgggccggaggaaagcgggaagactcatcggagcgtgtggatttgagccgccgcattttttaaccctagatctcgaaatgcatcgtgattcctgtccattggactgtaaggtttatgtaggcaatcttggaaacaatggcaacaagacggaattggaacgggcttttggctactatggaccactccgaagtgtgtgggttgctagaaacccacccggctttgcttttgttgaatttgaagatccccgagatgcagctgatgcagtccgagagctagatggaagaacactatgtggctgccgtgtaagagtggaactgtcgaatggtgaaaaaagaagtagaaatcgtggcccacctccctcttggggtcgtcgccctcgagatgattatcgtaggaggagtcctccacctcgtcgcagatctccaagaaggagaagcttctctcgcagccggagcaggtccctttctagagataggagaagagagagatcgctgtctcgggagagaaatcacaagccgtcccgatccttctctaggtctcgtagtcgatctaggtcaaatgaaaggaaatagaagacagtttgcaagagaagtggtgtacaggaaattacttcatttgacaggagtatgtacagaaaattcaagttttgtttgagacttcataagcttggtgcatttttaagatgttttagctgttcaaatctgtttgtctcttgaaacagtgacacaaaggtgtaattctctatggtttgaaatggatcatacgaggcatgtaataccaagaattgttactttacaatgttcccttaagcaaaattgaatttgctttgaacttttagttatgcacagactgataataaacctctaaacctgcccagcggaagtgtgtttttttttaaatttaaatacagaaacaactggcaaaaattgaactaagatttacttttttttccatagctgggatataggctgcagctatagttgaacaagcagtctttaaaaactgctgtgaaacacaggccatcagggaaaacgaaatgctgcactattaaattagaggtttttgaaaaatccaactctcatcctgggcagaggttgcctagttggtatagaatgttaagtttcaagaaagtttacctttgctttaggtcataagttccttatttgattgctgtatatggatacatggctgttcgtgacattctttatgtgcaaatttgtgatttcaaaaatgtcctgccagtttaagggtacattgtagagccgaactttgagttactgtgcaagatttttttttcatgctgtcatttgtaatatgttttgtgagaatccttgggattaaagttttggttacaaattgttaaaaaaaaaaaaaa

SEQ ID NO: 31 - protein, 164 aa, homo sapiens
MHRDSCPLDCKVYVGNLGNNGNKTELERAFGYYGPLRSVWVARNPPGFAFVEFEDPRDAADAVRELDGRTLCGCRVRVELSNGEKRSRNRGPPPSWGRRPRDDYRRRSPPPRRRSPRRRSFSRSRSRSLSRDRRRERSLSRERNHKPSRSFSRSRSRSRSNERK

SEQ ID NO: 32 - DNA, 1972 bp, homo sapiens
gaattcggcacgagggcgaccggcgcgtcgtgcggggctgcggcggagcctccttaaggaaggtgcaagaggttggcagcttcgattgaagcacatcgaccggcgacagcagccaggagtcatgagcgacagcggcgagcagaactacggcgagcgggaatcccgttctgcttccagaagtggaagtgctcacggatcggggaaatctgcaaggcatacccctgcaaggtctcgctccaaggaagattccaggcgttccagatcaaagtccaggtcccgatctgaatctaggtctagatccagaagaagctcccgaaggcattatacccggtcacggtctcgctcccgctcccatagacgatcacgtagcaggtcttacagtcgagattatcgtagacggcacagccacagccattctcccatgtctactcgcaggcgtcatgttgggaatcgggcaaatcctgatcctaactgttgtcttggagtatttgggctgagcttgtacaccacagaaagagatctaagagaagtgttctctaaatatggtcccattgccgatgtgtctattgtatatgaccagcagtctaggcgttcaagaggatttgcctttgtatattttgaaaatgtagatgatgccaaggaagctaaagaacgtgccaatggaatggagcttgatgggcgtaggatcagagttgatttctctataacaaaaagaccacatacgccaacaccaggaatttacatggggagacctacctatggcagctctcgccgtcgggattactatgacagaggatatgatcggggctatgatgatcgggactactatagcagatcatacagaggaggaggtggaggaggaggaggatggagagctgcccaagacagggatcagatttatagaaggcggtcaccttctccttactatagtcgtggaggatacagatcacgttccagatctcgatcatactcacctcgtcgctattaaagcatgaagactttctgaaacctgccctagagctgggatattgtttgtgggcaatattttttattgtctcttgtttaaaaagtgaacagtgcctagtgaagttaggtgacttttacaccttttacgatgactacttttggtggagttgaaatgctgttttcattctgcatttgtgtagtttggtgctttgttccaagttaagtgttttcagaaaagtatgttttgcatgtatttttttacagtctaaattttgactgctgagaagtttctattgtacaaaacttcatttaaaaggtttttctactgaatccagggtattctgaagatcgaagcctgtgtaaaatgctaccaaatggcaaaaagcaacaataaacagtttgatttttacttttctttctaacatatcaatgcttagcagaactattcagattgtcagtagtaaatttaaagacaaatgcccgttttcctccagtccatgaaacataccatacttatatacctgcaactaagtgtttaaaattatgctctgtaactctgtactgctagtattagaactaaaaatcttaaaatacagccagtgcttaatgcttatatcaatgtggatttgtcggcttttatgtaatctgtaatatgtatagcaggaaatacgaagagttacacagtgtatgccttaaaaggctgtttcttaaaggtgttacaaggggataatggtatttcaactagttatcagcaagtgacaatacattccaccacaaatacactcttgttcttctagcttttagactatatgaaaaaaccgggtgcttcaaagtacatgataagggaacactatacctgtcatggatgaactgaagactttgcctgttcattttttaaatattattttcaggtcctttgcttaccaaaggaggcccaatttcactcaaatgttttgagaactgtgtttaaataaacgcaaatgaaaagaaaaaaaaaaaaaaaaaa

SEQ ID NO: 33 - protein, 288 aa, homo sapiens
MSDSGEQNYGERESRSASRSGSAHGSGKSARHTPARSRSKEDSRRSRSKSRSRSESRSRSRRSSRRHYTRSRSRSRSHRRSRSRSYSRDYRRRHSHSHSPMSTRRRHVGNRANPDPNCCLGVFGLSLYTTERDLREVFSKYGPIADVSIVYDQQSRRSRGFAFVYFENVDDAKEAKERANGMELDGRRIRVDFSITKRPHTPTPGIYMGRPTYGSSRRRDYYDRGYDRGYDDRDYYSRSYRGGGGGGGGWRAAQDRDQIYRRRSPSPYYSRGGYRSRSRSRSYSPRRY

SEQ ID NO: 34 - DNA, 904 bp, homo sapiens
ggaattccgtcgacggcagcggcggcggcgggtgggaaatggcggagtatctggcctccatcttcggcaccgagaaagacaaagtcaactgttcattttatttcaaaattggagcatgtcgtcatggagacaggtgctctcggttgcacaataaaccgacgtttagccagaccattgccctcttgaacatttaccgtaaccctcaaaactcttcccagtctgctgacggtttgcgctgtgccgtgagcgatgtggagatgcaggaacactatgatgagttttttgaggaggtttttacagaaatggaggagaagtatggggaagtagaggagatgaacgtctgtgacaacctgggagaccacctggtggggaacgtgtacgtcaagtttcgccgtgaggaagatgcggaaaaggctgtgattgacttgaataaccgttggtttaatggacagccgatccacgccgagctgtcacccgtgacggacttcagagaagcctgctgccgtcagtatgagatgggagaatgcacacgaggcggcttctgcaacttcatgcatttgaagcccatttccagagagctgcggcgggagctgtatggccgccgtcgcaagaagcatagatcaagatcccgatcccgggagcgtcgttctcggtctagagaccgtggtcgtggcggtggcggtggcggtggtggaggtggcggcggacgggagcgtgacaggaggcggtcgagagatcgtgaaagatctgggcgattctgagccatgccatttttaccttatgtctgctagaaagtgttgtagttgattgaccaaaccagttcataaggggaattttttaaaaaacaacaaaaaaaaaacatacaaagatgggtttctgaataaaaatttgtagtgataacagt

SEQ ID NO: 35 - protein, 240 aa, homo sapiens
MAEYLASIFGTEKDKVNCSFYFKIGACRHGDRCSRLHNKPTFSQTIALLNIYRNPQNSSQSADGLRCAVSDVEMQEHYDEFFEEVFTEMEEKYGEVEEMNVCDNLGDHLVGNVYVKFRREEDAEKAVIDLNNRWFNGQPIHAELSPVTDFREACCRQYEMGECTRGGFCNFMHLKPISRELRRELYGRRRKKHRSRSRSRERRSRSRDRGRGGGGGGGGGGGGRERDRRRSRDRERSGRF

SEQ ID NO: 36 - DNA, 2090 bp, homo sapiens
gttggcgaggagtttcctgtttcccccgcagcgctgagttgaagttgagtgagtcactcgcgcgcacggagcgacgacacccccgcgcgtgcacccgctcgggacaggagccggactcctgtgcagcttccctcggccgccgggggcctccccgcgcctcgccggcctccaggcccctcctggctggcgagcgggcgccacatctggcccgcacatctgcgctgccggcccggcgcggggtccggagagggcgcggcgcggagcgcagccaggggtccgggaaggcgccgtccgtgcgctgggggctcggtctatgacgagcagcggggtctgccatgggtcgggggctgctcaggggcctgtggccgctgcacatcgtcctgtggacgcgtatcgccagcacgatcccaccgcacgttcagaagtcggttaataacgacatgatagtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggaaaaaaaaaagcctggtgagactttcttcatgtgttcctgtagctctgatgagtgcaatgacaacatcatcttctcagaagaatataacaccagcaatcctgacttgttgctagtcatatttcaagtgacaggcatcagcctcctgccaccactgggagttgccatatctgtcatcatcatcttctactgctaccgcgttaaccggcagcagaagctgagttcaacctgggaaaccggcaagacgcggaagctcatggagttcagcgagcactgtgccatcatcctggaagatgaccgctctgacatcagctccacgtgtgccaacaacatcaaccacaacacagagctgctgcccattgagctggacaccctggtggggaaaggtcgctttgctgaggtctataaggccaagctgaagcagaacacttcagagcagtttgagacagtggcagtcaagatctttccctatgaggagtatgcctcttggaagacagagaaggacatcttctcagacatcaatctgaagcatgagaacatactccagttcctgacggctgaggagcggaagacggagttggggaaacaatactggctgatcaccgccttccacgccaagggcaacctacaggagtacctgacgcggcatgtcatcagctgggaggacctgcgcaagctgggcagctccctcgcccgggggattgctcacctccacagtgatcacactccatgtgggaggcccaagatgcccatcgtgcacagggacctcaagagctccaatatcctcgtgaagaacgacctaacctgctgcctgtgtgactttgggctttccctgcgtctggaccctactctgtctgtggatgacctggctaacagtgggcaggtgggaactgcaagatacatggctccagaagtcctagaatccaggatgaatttggagaatgctgagtccttcaagcagaccgatgtctactccatggctctggtgctctgggaaatgacatctcgctgtaatgcagtgggagaagtaaaagattatgagcctccatttggttccaaggtgcgggagcacccctgtgtcgaaagcatgaaggacaacgtgttgagagatcgagggcgaccagaaattcccagcttctggctcaaccaccagggcatccagatggtgtgtgagacgttgactgagtgctgggaccacgacccagaggcccgtctcacagcccagtgtgtggcagaacgcttcagtgagctggagcatctggacaggctctcggggaggagctgctcggaggagaagattcctgaagacggctccctaaacactaccaaatagctcttatggggcaggctgggcatgtccaaagaggctgcccctctcaccaaa

SEQ ID NO: 37 - protein, 567 aa, homo sapiens
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSSTWETGKTRKLMEFSEHCAIILEDDRSDISSTCANNINHNTELLPIELDTLVGKGRFAEVYKAKLKQNTSEQFETVAVKIFPYEEYASWKTEKDIFSDINLKHENILQFLTAEERKTELGKQYWLITAFHAKGNLQEYLTRHVISWEDLRKLGSSLARGIAHLHSDHTPCGRPKMPIVHRDLKSSNILVKNDLTCCLCDFGLSLRLDPTLSVDDLANSGQVGTARYMAPEVLESRMNLENAESFKQTDVYSMALVLWEMTSRCNAVGEVKDYEPPFGSKVREHPCVESMKDNVLRDRGRPEIPSFWLNHQGIQMVCETLTECWDHDPEARLTAQCVAERFSELEHLDRLSGRSCSEEKIPEDGSLNTTK

SEQ ID NO: 38 - DNA, 1725 bp, homo sapiens
cgcctcttcacggcactgggatccgcatctgcctgggatcatcaagccctagaagctgggtttctttaaattagggctgccgttttctgtttctccctgggctgcggaaagccagaagattttatctagcttatacaaggctgctggtgttccctctttttttccacgagggtgtttttggctgcaattgcatgaaatcccaatggtgtagaccagtggcgatggatctaggagtttaccaactgagacatttttcaatttctttcttgtcatccttgctggggactgaaaacgcttctgtgagacttgataatagctcctctggtgcaagtgtggtagctattgacaacaaaatcgagcaagctatggatctagtgaaaagccatttgatgtatgcggtcagagaagaagtggaggtcctcaaagagcaaatcaaagaactaatagagaaaaattcccagctggagcaggagaacaatctgctgaagacactggccagtcctgagcagcttgcccagtttcaggcccagctgcagactggctccccccctgccaccacccagccacagggcaccacacagccccccgcccagccagcatcgcagggctcaggaccaaccgcatagctgcctatgcccccgcagaactggctgctgcgtgtgaactgaacagacggagaagatgtgctagggagaatctgcctccacagtcacccatttcattgctcgctgcgaaagagacgtgagactgacatatgccattatctcttttccagtattaaacactcatatgcttatggcttggagaaatttcttagttgggtgaattaaaggttaatccgagaattagcatggatataccgggacctcatgcagcttggcagatatctgagaaatggtttaattcatgctcaggagctgtgtgcctttccatcccttccggctccctacccctcacttccaagggttctctctcctgcttgcgcttagtgtcctacatggggttgtgaagcgatggagctcctcactggactcgcctctctcctctcctccccccaggaggaacttgaaaggagggtaaaaagactaaaatgagggggaacagagttcactgtacaaatttgacaactgtcaccaaaattcataaaaaacaatagtactgtgcctctttcttctcaaacaatggatgacacaaaactatgagagtgacaaaatggtgacaggtagctgggacctaggctatcttaccatgaaggttgttttgcttattgtatatttgtgtatgtagtgtaactattttgtacaatagaggactgtaactactatttaggttgtacagattgaaatttagttgtttcattggctgtctgaggaggtgtggacttttatatatagatctacataaaaactgctacatgacaaaaaccacacctaaaccccttttaagaatttggcacagttactcactttgtgtaatctgaaatctagctgctgaatacgctgaagtaaatccttgttcactgaagtctttcaattgagctggttgaatactttgaaaaatgctcagttctaactaatgaaatggatttcccagtaggggtttctgcatatcacctgtatagtagttatatgcatatgtttctgtgcatgttctctacacaattgtaaggtgtcactgtatttaactgttgcacttgtcaactttcaataaagcatataaatgttg

SEQ ID NO: 39 - protein, 144 aa, homo sapiens
MKSQWCRPVAMDLGVYQLRHFSISFLSSLLGTENASVRLDNSSSGASVVAIDNKIEQAMDLVKSHLMYAVREEVEVLKEQIKELIEKNSQLEQENNLLKTLASPEQLAQFQAQLQTGSPPATTQPQGTTQPPAQPASQGSGPTA

SEQ ID NO: 40 - DNA, 1693 bp, homo sapiens
gcagatccgctgtacttgcgggcgctacagtatgtcaatcgcttgccccagcacagtgggctccgtggcttaagacttgaaccaagtaaacgaagttctcttactgagaagtctcagtttcaaaagagcttctcctcatcaactggggatgattacagttcttcctaaaaaagcctacttgatgtgaagacaatgaggatgaagacctttatggtgatccacttccacttaataggatggctgctctttttctaaagaggttaacactacaaactgtaaagtctgaaaatagttgcattagatgttttggtaaacacatcctgcaaaagacagcaccagcacagttgtcccctattgcttctgccccaagactctccttcctaattcatgcaaaagcctttagtaccgctgaagacacccagaatgaaggaaaaaagacaaaaaagaataaaacagcttttagtaacgttggaagaaaaattagtcagcgagttattcacttatttgatgagaagggcaatgatttgggaaacatgcaccgagcaaatgtgattagacttatggatgagcgagacctgcgactggttcaaaggaacaccagcacagaacctgcagagtatcagctcatgacaggattgcagatcctccaggagcggcagaggctgagggagatggagaaggcgaaccccaaaactggaccaaccctgagaaaggaactgattttgtcttcaaatattggacaacatgatttggacacaaagactaaacagattcagcagtggattaagaaaaaacacctagtccagattaccataaagaaaggaaaaaatgtagacgtgtcagaaaatgaaatggaggagatatttcatcaaatactccagactatgcctggaatagctacattctcatctaggccacaagctgttcaaggaggaaaagctttaatgtgtgttcttcgtgctttgagcaaaaatgaggagaaggcatataaagaaactcaagagacccaggaaagagacactttgaacaaagatcatggaaatgataaggaatcaaatgttctgcatcagtaattttaataaagaaaagcatgctctgagagaaaaaaaagctcgctccttggtctgcagtcctttaaacaaagcagtgcagttcttagccaagggtaagtactgcaactgtcgagagcatcttgtcttccacacagttgggtgactctccgttttgacacaaagataagccttgcccttgtttccttttgggagggatatatccactgagatgagaggccaaactccgtttttcacgagattttttgactttgagcttcattttcttcttgtcaggatcatgtacaacagcatgcctagtgagactttgtttcattgcaaatgttttgccacagccagcatgttcacacacaaaagggcggctttcctcatggaaggagaggatatggctttggagattaaacacagttgtataggttcttccacagccttctcttggacagcgacataatcccttctggggcatgagtttatgtgttgcttaaggaacttgcgttaaagttttccggcaacttcacatggattcctttgaatgagttcaaatgttcccatgctaagctgagtctgtgccatagcaaaccatgatatagcaagtctccagaatgtgtacgaatcaatactcc

SEQ ID NO: 41 - protein, 278 aa, homo sapiens
MAALFLKRLTLQTVKSENSCIRCFGKHILQKTAPAQLSPIASAPRLSFLIHAKAFSTAEDTQNEGKKTKKNKTAFSNVGRKISQRVIHLFDEKGNDLGNMHRANVIRLMDERDLRLVQRNTSTEPAEYQLMTGLQILQERQRLREMEKANPKTGPTLRKELILSSNIGQHDLDTKTKQIQQWIKKKHLVQITIKKGKNVDVSENEMEEIFHQILQTMPGIATFSSRPQAVQGGKALMCVLRALSKNEEKAYKETQETQERDTLNKDHGNDKESNVLHQ

SEQ ID NO: 42 - DNA, 1776 bp, homo sapiens
cagcatggccaccaccgccacctgcacccgtttcaccgacgactaccagctcttcgaggagcttggcaagggtgctttctctgtggtccgcaggtgtgtgaagaaaacctccacgcaggagtacgcagcaaaaatcatcaataccaagaagttgtctgcccgggatcaccagaaactagaacgtgaggctcggatatgtcgacttctgaaacatccaaacatcgtgcgcctccatgacagtatttctgaagaagggtttcactacctcgtgtttgaccttgttaccggcggggagctgtttgaagacattgtggccagagagtactacagtgaagcagatgccagccactgtatacatcagattctggagagtgttaaccacatccaccagcatgacatcgtccacagggacctgaagcctgagaacctgctgctggcgagtaaatgcaagggtgccgccgtcaagctggctgattttggcctagccatcgaagtacagggagagcagcaggcttggtttggttttgctggcaccccaggttacttgtcccctgaggtcttgaggaaagatccctatggaaaacctgtggatatctgggcctgcggggtcatcctgtatatcctcctggtgggctatcctcccttctgggatgaggatcagcacaagctgtatcagcagatcaaggctggagcctatgatttcccatcaccagaatgggacacggtaactcctgaagccaagaacttgatcaaccagatgctgaccataaacccagcaaagcgcatcacggctgaccaggctctcaagcacccgtgggtctgtcaacgatccacggtggcatccatgatgcatcgtcaggagactgtggagtgtttgcgcaagttcaatgcccggagaaaactgaagggtgccatcctcacgaccatgcttgtctccaggaacttctcagctgccaaaagcctattgaacaagaagtcggatggcggtgtcaagccacagagcaacaacaaaaacagtctcgtaagcccagcccaagagcccgcgcccttgcagacggccatggagccacaaaccactgtggtacacaacgctacagatgggatcaagggctccacagagagctgcaacaccaccacagaagatgaggacctcaaagtgcgaaaacaggagatcattaagattacagaacagctgattgaagccatcaacaatggggactttgaggcctacacgaagatttgtgatccaggcctcacttcctttgagcctgaggcccttggtaacctcgtggaggggatggatttccataagttttactttgagaatctcctgtccaagaacagcaagcctatccataccaccatcctaaacccacacgtccacgtgattggggaggacgcagcgtgcatcgcctacatccgcctcacccagtacatcgacgggcagggtcggcctcgcaccagccagtcagaagagacccgggtctggcaccgtcgggatggcaagtggctcaatgtccactatcactgctcaggggcccctgccgcaccgctgcagtgagctcagccacaggggctttaggagattccagccggaggtccaaccttcgcagccagtggctctggagggcctgagtgacagcggcagtcctgtttgtttgaggtttaaaacaattcaattacaaaagcggcagcagccaatgcacgcccctgcatgcagccctcccgcccgcccttcgtgtctgtctctgctgtaccgaggtgttttttacattt

SEQ ID NO: 43 - protein, 518 aa, homo sapiens
MATTATCTRFTDDYQLFEELGKGAFSVVRRCVKKTSTQEYAAKIINTKKLSARDHQKLEREARICRLLKHPNIVRLHDSISEEGFHYLVFDLVTGGELFEDIVAREYYSEADASHCIHQILESVNHIHQHDIVHRDLKPENLLLASKCKGAAVKLADFGLAIEVQGEQQAWFGFAGTPGYLSPEVLRKDPYGKPVDIWACGVILYILLVGYPPFWDEDQHKLYQQIKAGAYDFPSPEWDTVTPEAKNLINQMLTINPAKRITADQALKHPWVCQRSTVASMMHRQETVECLRKFNARRKLKGAILTTMLVSRNFSAAKSLLNKKSDGGVKPQSNNKNSLVSPAQEPAPLQTAMEPQTTVVHNATDGIKGSTESCNTTTEDEDLKVRKQEIIKITEQLIEAINNGDFEAYTKICDPGLTSFEPEALGNLVEGMDFHKFYFENLLSKNSKPIHTTILNPHVHVIGEDAACIAYIRLTQYIDGQGRPRTSQSEETRVWHRRDGKWLNVHYHCSGAPAAPLQ

SEQ ID NO: 44 - DNA, 1377 bp, homo sapiens
ggccttcagccctctgtggtcccctctccccggggggctttgggattcttgtcaagctccttcaagagcctgcaagcacttaaccagccacccagagttccctcactgaagatctgagcatgacccgggacgaggcactgccggactctcattctgcacaggacttctatgagaattatgagcccaaagagatcctgggcaggggcgttagcagtgtggtcaggcgatgcatccacaagcccacgagccaggagtacgccgtgaaggtcatcgacgtcaccggtggaggcagcttcagcccggaggaggtgcgggagctgcgagaagccacgctgaaggaggtggacatcctgcgcaaggtctcagggcaccccaacatcatacagctgaaggacacttatgagaccaacactttcttcttcttggtgtttgacctgatgaagagaggggagctctttgactacctcactgagaaggtcaccttgagtgagaaggaaaccagaaagatcatgcgagctctgctggaggtgatctgcaccttgcacaaactcaacatcgtgcaccgggacctgaagcccgagaacattctcttggatgacaacatgaacatcaagctcacagactttggcttttcctgccagctggagccgggagagaggctgcgagaggtctgcgggacccccagttacctggcccctgagattatcgagtgctccatgaatgaggaccacccgggctacgggaaagaggtggacatgtggagcactggcgtcatcatgtacacgctgctggccggctccccgcccttctggcaccggaagcagatgctgatgctgaggatgatcatgagcggcaactaccagtttggctcgcccgagtgggatgattactcggacaccgtgaaggacctggtctcccgattcctggtggtgcaaccccagaaccgctacacagcggaagaggccttggcacaccccttcttccagcagtacttggtggaggaagtgcggcacttcagcccccgggggaagttcaaggtgatcgctctgaccgtgctggcttcagtgcggatctactaccagtaccgccgggtgaagcctgtgacccgggagatcgtcatccgagacccctatgccctccggcctctgcgccggctcatcgacgcctacgctttccgaatctatggccactgggtgaagaaggggcagcagcagaaccgggcagcccttttcgagaacacacccaaggccgtgctcctctccctggccgaggaggactactgaggggctggccagtcagggagggctagggggcaggtggggaggggaagccatggaaatacaagtcaaaggggtaaaaaaaaaaaaaaaaaaaaaa

SEQ ID NO: 45 - protein, 387 aa, homo sapiens
MTRDEALPDSHSAQDFYENYEPKEILGRGVSSVVRRCIHKPTSQEYAVKVIDVTGGGSFSPEEVRELREATLKEVDILRKVSGHPNIIQLKDTYETNTFFFLVFDLMKRGELFDYLTEKVTLSEKETRKIMRALLEVICTLHKLNIVHRDLKPENILLDDNMNIKLTDFGFSCQLEPGERLREVCGTPSYLAPEIIECSMNEDHPGYGKEVDMWSTGVIMYTLLAGSPPFWHRKQMLMLRMIMSGNYQFGSPEWDDYSDTVKDLVSRFLVVQPQNRYTAEEALAHPFFQQYLVEEVRHFSPRGKFKVIALTVLASVRIYYQYRRVKPVTREIVIRDPYALRPLRRLIDAYAFRIYGHWVKKGQQQNRAALFENTPKAVLLSLAEEDY

SEQ ID NO: 46 - DNA, 2360 bp, homo sapiens
gctacgcgggccacgctgctggctggcctgacctaggcgcgcggggtcgggcggccgcgcgggcgggctgagtgagcaagacaagacactcaagaagagcgagctgcgcctgggtcccggccaggcttgcacgcagaggcgggcggcagacggtgcccggcggaatctcctgagctccgccgcccagctctggtgccagcgcccagtggccgccgcttcgaaagtgactggtgcctcgccgcctcctctcggtgcgggaccatgaagctgctgccgtcggtggtgctgaagctctttctggctgcagttctctcggcactggtgactggcgagagcctggagcggcttcggagagggctagctgctggaaccagcaacccggaccctcccactgtatccacggaccagctgctacccctaggaggcggccgggaccggaaagtccgtgacttgcaagaggcagatctggaccttttgagagtcactttatcctccaagccacaagcactggccacaccaaacaaggaggagcacgggaaaagaaagaagaaaggcaaggggctagggaagaagagggacccatgtcttcggaaatacaaggacttctgcatccatggagaatgcaaatatgtgaaggagctccgggctccctcctgcatctgccacccgggttaccatggagagaggtgtcatgggctgagcctcccagtggaaaatcgcttatatacctatgaccacacaaccatcctggccgtggtggctgtggtgctgtcatctgtctgtctgctggtcatcgtggggcttctcatgtttaggtaccataggagaggaggttatgatgtggaaaatgaagagaaagtgaagttgggcatgactaattcccactgagagagacttgtgctcaaggaatcggctggggactgctacctctgagaagacacaaggtgatttcagactgcagaggggaaagacttccatctagtcacaaagactccttcgtccccagttgccgtctaggattgggcctcccataattgctttgccaaaataccagagccttcaagtgccaaacagagtatgtccgatggtatctgggtaagaagaaagcaaaagcaagggaccttcatgcccttctgattcccctccaccaaaccccacttcccctcataagtttgtttaaacacttatcttctggattagaatgccggttaaattccatatgctccaggatctttgactgaaaaaaaaaaagaagaagaagaaggagagcaagaaggaaagatttgtgaactggaagaaagcaacaaagattgagaagccatgtactcaagtaccaccaagggatctgccattgggaccctccagtgctggatttgatgagttaactgtgaaataccacaagcctgagaactgaattttgggacttctacccagatggaaaaataacaactatttttgttgttgttgtttgtaaatgcctcttaaattatatatttattttattctatgtatgttaatttatttagtttttaacaatctaacaataatatttcaagtgcctagactgttactttggcaatttcctggccctccactcctcatccccacaatctggcttagtgccacccacctttgccacaaagctaggatggttctgtgacccatctgtagtaatttattgtctgtctacatttctgcagatcttccgtggtcagagtgccactgcgggagctctgtatggtcaggatgtaggggttaacttggtcagagccactctatgagttggacttcagtcttgcctaggcgattttgtctaccatttgtgttttgaaagcccaaggtgctgatgtcaaagtgtaacagatatcagtgtctccccgtgtcctctccctgccaagtctcagaagaggttgggcttccatgcctgtagctttcctggtccctcacccccatggccccaggccacagcgtgggaactcactttcccttgtgtcaagacatttctctaactcctgccattcttctggtgctactccatgcaggggtcagtgcagcagaggacagtctggagaaggtattagcaaagcaaaaggctgagaaggaacagggaacattggagctgactgttcttggtaactgattacctgccaattgctaccgagaaggttggaggtggggaaggctttgtataatcccacccacctcaccaaaacgatgaaggtatgctgtcatggtcctttctggaagtttctggtgccatttctgaactgttacaacttgtatttccaaacctggttcatatttatactttgcaatccaaataaagataacccttattccataaaaaaaaaaaaaaaaaa

SEQ ID NO: 47 - protein, 208 aa, homo sapiens
MKLLPSVVLKLFLAAVLSALVTGESLERLRRGLAAGTSNPDPPTVSTDQLLPLGGGRDRKVRDLQEADLDLLRVTLSSKPQALATPNKEEHGKRKKKGKGLGKKRDPCLRKYKDFCIHGECKYVKELRAPSCICHPGYHGERCHGLSLPVENRLYTYDHTTILAVVAVVLSSVCLLVIVGLLMFRYHRRGGYDVENEEKVKLGMTNSH

SEQ ID NO: 48 - DNA, 4119 bp, homo sapiens
ctggagagcctgctgcccgcccgcccgtaaaatggtcccctcggctggacagctcgccctgttcgctctgggtattgtgttggctgcgtgccaggccttggagaacagcacgtccccgctgagtgcagacccgcccgtggctgcagcagtggtgtcccattttaatgactgcccagattcccacactcagttctgcttccatggaacctgcaggtttttggtgcaggaggacaagccagcatgtgtctgccattctgggtacgttggtgcacgctgtgagcatgcggacctcctggccgtggtggctgccagccagaagaagcaggccatcaccgccttggtggtggtctccatcgtggccctggctgtccttatcatcacatgtgtgctgatacactgctgccaggtccgaaaacactgtgagtggtgccgggccctcatctgccggcacgagaagcccagcgccctcctgaagggaagaaccgcttgctgccactcagaaacagtggtctgaagagcccagaggaggagtttggccaggtggactgtggcagatcaataaagaaaggcttcttcaggacagcactgccagagatgcctgggtgtgccacagaccttcctacttggcctgtaatcacctgtgcagccttttgtgggccttcaaaactctgtcaagaactccgtctgcttggggttattcagtgtgacctagagaagaaatcagcggaccacgatttcaagacttgttaaaaaagaactgcaaagagacggactcctgttcacctaggtgaggtgtgtgcagcagttggtgtctgagtccacatgtgtgcagttgtcttctgccagccatggattccaggctatatatttctttttaatgggccacctccccacaacagaattctgcccaacacaggagatttctatagttattgttttctgtcatttgcctactggggaagaaagtgaaggaggggaaactgtttaatatcacatgaagaccctagctttaagagaagctgtatcctctaaccacgagactctcaaccagcccaacatcttccatggacacatgacattgaagaccatcccaagctatcgccacccttggagatgatgtcttatttattagatggataatggttttatttttaatctcttaagtcaatgtaaaaagtataaaaccccttcagacttctacattaatgatgtatgtgttgctgactgaaaagctatactgattagaaatgtctggcctcttcaagacagctaaggcttgggaaaagtcttccagggtgcggagatggaaccagaggctgggttactggtaggaataaaggtaggggttcagaaatggtgccattgaagccacaaagccggtaaatgcctcaatacgttctgggagaaaacttagcaaatccatcagcagggatctgtcccctctgttggggagagaggaagagtgtgtgtgtctacacaggataaacccaatacatattgtactgctcagtgattaaatgggttcacttcctcgtgagccctcggtaagtatgtttagaaatagaacattagccacgagccataggcatttcaggccaaatccatgaaagggggaccagtcatttattttccattttgttgcttggttggtttgttgctttatttttaaaaggagaagtttaactttgctatttattttcgagcactaggaaaactattccagtaatttttttttcctcatttccattcaggatgccggctttattaacaaaaactctaacaagtcacctccactatgtgggtcttcctttcccctcaagagaaggagcaattgttcccctgacatctgggtccatctgacccatggggcctgcctgtgagaaacagtgggtcccttcaaatacatagtggatagctcatccctaggaattttcattaaaatttggaaacagagtaatgaagaaataatatataaactccttatgtgaggaaatgctactaatatctgaaaagtgaaagatttctatgtattaactcttaagtgcacctagcttattacatcgtgaaaggtacatttaaaatatgttaaattggcttgaaattttcagagaattttgtcttcccctaattcttcttccttggtctggaagaacaatttctatgaattttctctttattttttttttataattcagacaattctatgacccgtgtcttcatttttggcactcttatttaacaatgccacacctgaagcacttggatctgttcagagctgaccccctagcaacgtagttgacacagctccaggtttttaaattactaaaataagttcaagtttacatcccttgggccagatatgtgggttgaggcttgactgtagcatcctgcttagagaccaatcaatggacactggtttttagacctctatcaatcagtagttagcatccaagagactttgcagaggcgtaggaatgaggctggacagatggcggaacgagaggttccctgcgaagacttgagatttagtgtctgtgaatgttctagttcctaggtccagcaagtcacacctgccagtgccctcatccttatgcctgtaacacacatgcagtgagaggcctcacatatacgcctccctagaagtgccttccaagtcagtcctttggaaaccagcaggtctgaaaaagaggctgcatcaatgcaagcctggttggaccattgtccatgcctcaggatagaacagcctggcttatttggggatttttcttctagaaatcaaatgactgataagcattggctccctctgccatttaatggcaatggtagtctttggttagctgcaaaaatactccatttcaagttaaaaatgcatcttctaatccatctctgcaagctccctgtgtttccttgccctttagaaaatgaattgttcactacaattagagaatcatttaacatcctgacctggtaagctgccacacacctggcagtggggagcatcgctgtttccaatggctcaggagacaatgaaaagcccccatttaaaaaaataacaaacattttttaaaaggcctccaatactcttatggagcctggatttttcccactgctctacaggctgtgactttttttaagcatcctgacaggaaatgttttcttctacatggaaagatagacagcagccaaccctgatctggaagacagggccccggctggacacacgtggaaccaagccagggatgggctggccattgtgtccccgcaggagagatgggcagaatggccctagagttcttttccctgagaaaggagaaaaagatgggattgccactcacccacccacactggtaagggaggagaatttgtgcttctggagcttctcaagggattgtgttttgcaggtacagaaaactgcctgttatcttcaagccaggttttcgagggcacatgggtcaccagttgctttttcagtcaatttggccgggatggactaatgaggctctaacactgctcaggagacccctgccctctagttggttctgggctttgatctcttccaacctgcccagtcacagaaggaggaatgactcaaatgcccaaaaccaagaacacattgcagaagtaagacaaacatgtatatttttaaatgttctaacataagacctgttctctctagccattgatttaccaggctttctgaaagatctagtggttcacacagagagagagagagtactgaaaaagcaactcctcttcttagtcttaataatttactaaaatggtcaacttttcattatctttattataataaacctgatgcttttttttagaactccttactctgatgtctgtatatgttgcactgaaaaggttaatatttaatgttttaatttattttgtgtggtaagttaattttgatttctgtaatgtgttaatgtgattagcagttattttccttaatatctgaattatacttaaagagtagtgagcaatataagacgcaattgtgtttttcagtaatgtgcattgttattgagttgtactgtaccttatttggaaggatgaaggaatgaacctttttttcctaaaaaaaaaaaaaaaaaaaaaaaaaaaaaaaaa

SEQ ID NO: 49 - protein, 160 aa, homo sapiens
MVPSAGQLALFALGIVLAACQALENSTSPLSADPPVAAAVVSHFNDCPDSHTQFCFHGTCRFLVQEDKPACVCHSGYVGARCEHADLLAVVAASQKKQAITALVVVSIVALAVLIITCVLIHCCQVRKHCEWCRALICRHEKPSALLKGRTACCHSETVV

SEQ ID NO: 50 - DNA, 4259 bp, homo sapiens
atggcgaagatcgccaagactcacgaagatattgaagcacagattcgagaaattcaaggcaagaaggcagctcttgatgaagctcaaggagtgggcctcgattctacaggttattatgaccaggaaatttatggtggaagtgacagcagatttgctggatacgtgacatcaattgctgcaactgaacttgaagatgatgacgatgactattcatcatctacgagtttgcttggtcagaagaagccaggatatcatgcccctgtggcattgcttaatgatataccacagtcaacagaacagtatgatccatttgctgagcacagacctccaaagattgcagaccgggaagatgaatacaaaaagcataggcggaccatgataatttccccagagcgtcttgatccttttgcagatggagggaagacccctgatcctaaaatgaatgttaggacttacatggatgtaatgcgagaacaacacttgactaaagaagaacgagaaattaggcaacagctagcagaaaaagctaaagctggagaactaaaagtcgtcaatggagcagcagcgtcccagcctccatcaaaacgaaaacggcgttgggatcaaacagctgatcagactcctggtgccactcccaaaaaactatcaagttgggatcaggcagagacccctgggcatactccttccttaagatgggatgagacaccaggtcgtgcaaagggaagcgagactcctggagcaaccccaggctcaaaaatatgggatcctacacctagccacacaccagcgggagctgctactcctggacgaggtgatacaccaggccatgcgacaccaggccatggaggcgcaacttccagtgctcgtaaaaacagatgggatgaaacccccaaaacagagagagatactcctgggcatggaagtggatgggctgagactcctcgaacagatcgaggtggagattctattggtgaaacaccgactcctggagccagtaaaagaaaatcacggtgggatgaaacaccagctagtcagatgggtggaagcactccagttctgacccctggaaagacaccaattggcacaccagccatgaacatggctacccctactccaggtcacataatgagtatgactcctgaacagcttcaggcttggcggtgggaaagagaaattgatgagagaaatcgcccactttctgatgaggaattagatgctatgttcccagaaggatataaggtacttcctcctccagctggttatgttcctattcgaactccagctcgaaagctgacagctactccaacacctttgggtggtatgactggtttccacatgcaaactgaagatcgaactatgaaaagtgttaatgaccagccatctggaaatcttccatttttaaaacctgatgatattcaatactttgataaactattggttgatgttgatgaatcaacacttagtccagaagagcaaaaagagagaaaaataatgaagttgcttttaaaaattaagaatggaacaccaccaatgagaaaggctgcattgcgtcagattactgataaagctcgtgaatttggagctggtcctttgtttaatcagattcttcctctgctgatgtctcctacacttgaggatcaagagcgtcatttacttgtgaaagttattgataggatactgtacaaacttgatgacttagttcgtccatatgtgcataagatcctcgtggtcattgaaccgctattgattgatgaagattactatgctagagtggaaggcctagagatcatttctaatttggcaaaggctgctggtctggctactatgatctctaccatgagacctgatatagataacatggatgagtatgtccgtaacacaacagctagagcttttgctgttgtagcctctgccctgggcattccttctttattgcccttcttaaaagctgtgtgcaaaagcaagaagtcctggcaagcgagacacactggtattaagattgtacaacagatagctattcttatgggctgtgccatcttgccacatcttagaagtttagttgaaatcattgaacatggtcttgtggatgagcagcagaaagttcggaccatcagtgctttggccattgctgccttggctgaagcagcaactccttatggtatcgaatcttttgattctgtgttaaagcctttatggaagggtatccgccaacacagaggaaagggtttggctgctttcttgaaggctattgggtatcttattcctcttatggatgcagaatatgccaactactatactagagaagtgatgttaatccttattcgagaattccagtctcctgatgaggaaatgaaaaaaattgtgctgaaggtggtaaaacagtgttgtgggacagatggtgtagaagcaaactacattaaaacagagattcttcctcccttttttaaacacttctggcagcacaggatggctttggatagaagaaattaccgacagttagttgatactactgtggagttggcaaacaaagtaggtgcagcagaaattatatccaggattgtggatgatctgaaagatgaagccgaacagtacagaaaaatggtgatggagacaattgagaaaattatgggcaatttgggagcagcagatattgatcataaacttgaagaacaactgattgatggtattctttatgctttccaagaacagactacagaggactcagtaatgttgaacggctttggcacagtggttaatgctcttggcaaacgagtcaaaccatacttgcctcagatctgtggtacagttttgtggcgtttaaataacaaatctgctaaagttaggcaacaggcagctgacttgatttctcgaactgctgttgtcatgaagacttgtcaagaggaaaaattgatgggacacttgggtgttgtattgtatgagtatttgggtgaagagtaccctgaagtattgggcagcattcttggagcactgaaggccattgtaaatgtcataggtatgcataagatgactccaccaattaaagatctgctgcctagactcacccccatcttaaagaacagacatgaaaaagtacaagagaattgtattgatcttgttggtcgtattgctgacaggggagctgaatatgtatctgcaagagagtggatgaggatttgctttgagcttttagagctcttaaaagcccacaaaaaggctattcgtagagccacagtcaacacatttggttatattgcaaaggccattggccctcatgatgtattggctacacttctgaacaacctcaaagttcaagaaaggcagaacagagtttgtaccactgtagcaatagctattgttgcagaaacatgttcaccctttacagtactccctgccttaatgaatgaatacagagttcctgaactgaatgttcaaaatggagtgttaaaatcgctttccttcttgtttgaatatattggtgaaatgggaaaagactacatttatgccgtaacaccgttacttgaagatgctttaatggatagagaccttgtacacagacagacggctagtgcagtggtacagcacatgtcacttggggtttatggatttggttgtgaagattcgctgaatcacttgttgaactatgtatggcccaatgtatttgagacatctcctcatgtaattcaggcagttatgggagccctagagggcctgagagttgctattggaccatgtagaatgttgcaatattgtttacagggtctgtttcacccagcccggaaagtcagagatgtatattggaaaatttacaactccatctacattggttcccaggacgctctcatagcacattacccaagaatctacaacgatgataagaacacctatattcgttatgaacttgactatatcttataattttattgtttattttgtgtttaatgcacagctacttcacaccttaaacttgctttgatttggtgatgtaaacttttaaacattgcagttcagtgtagaactggtcatagaggaagagctagaaatccagtagcatgatttttaaataacctgtctttgtttttgatgttaaacagtaaatgccagtagtgaccaagaacacagtgattatatacactatactggagggatttcatttttaattcatctttatgaagatttagaactcattccttgtgtttaaagggaatgtttaattgagaaataaacatttgtgtacaaaatgctaaaaaaaaaaaaaaaaa

SEQ ID NO: 51 - protein, 1304 aa, homo sapiens
MAKIAKTHEDIEAQIREIQGKKAALDEAQGVGLDSTGYYDQEIYGGSDSRFAGYVTSIAATELEDDDDDYSSSTSLLGQKKPGYHAPVALLNDIPQSTEQYDPFAEHRPPKIADREDEYKKHRRTMIISPERLDPFADGGKTPDPKMNVRTYMDVMREQHLTKEEREIRQQLAEKAKAGELKVVNGAAASQPPSKRKRRWDQTADQTPGATPKKLSSWDQAETPGHTPSLRWDETPGRAKGSETPGATPGSKIWDPTPSHTPAGAATPGRGDTPGHATPGHGGATSSARKNRWDETPKTERDTPGHGSGWAETPRTDRGGDSIGETPTPGASKRKSRWDETPASQMGGSTPVLTPGKTPIGTPAMNMATPTPGHIMSMTPEQLQAWRWEREIDERNRPLSDEELDAMFPEGYKVLPPPAGYVPIRTPARKLTATPTPLGGMTGFHMQTEDRTMKSVNDQPSGNLPFLKPDDIQYFDKLLVDVDESTLSPEEQKERKIMKLLLKIKNGTPPMRKAALRQITDKAREFGAGPLFNQILPLLMSPTLEDQERHLLVKVIDRILYKLDDLVRPYVHKILVVIEPLLIDEDYYARVEGLEIISNLAKAAGLATMISTMRPDIDNMDEYVRNTTARAFAVVASALGIPSLLPFLKAVCKSKKSWQARHTGIKIVQQIAILMGCAILPHLRSLVEIIEHGLVDEQQKVRTISALAIAALAEAATPYGIESFDSVLKPLWKGIRQHRGKGLAAFLKAIGYLIPLMDAEYANYYTREVMLILIREFQSPDEEMKKIVLKVVKQCCGTDGVEANYIKTEILPPFFKHFWQHRMALDRRNYRQLVDTTVELANKVGAAEIISRIVDDLKDEAEQYRKMVMETIEKIMGNLGAADIDHKLEEQLIDGILYAFQEQTTEDSVMLNGFGTVVNALGKRVKPYLPQICGTVLWRLNNKSAKVRQQAADLISRTAVVMKTCQEEKLMGHLGVVLYEYLGEEYPEVLGSILGALKAIVNVIGMHKMTPPIKDLLPRLTPILKNRHEKVQENCIDLVGRIADRGAEYVSAREWMRICFELLELLKAHKKAIRRATVNTFGYIAKAIGPHDVLATLLNNLKVQERQNRVCTTVAIAIVAETCSPFTVLPALMNEYRVPELNVQNGVLKSLSFLFEYIGEMGKDYIYAVTPLLEDALMDRDLVHRQTASAVVQHMSLGVYGFGCEDSLNHLLNYVWPNVFETSPHVIQAVMGALEGLRVAIGPCRMLQYCLQGLFHPARKVRDVYWKIYNSIYIGSQDALIAHYPRIYNDDKNTYIRYELDYIL

SEQ ID NO: 52 - DNA, 2321 bp, homo sapiens
ggtggatgctgcggcatcggaggaccctgctggtggaggaaatggttcacgcccgtccccgttccctttgcaggcttgctattgtgcgtctgtgattgacaagaccacgaggctgagcgcgccctggagatttttctataaatggcttaacaccccagtctagactatttgctcggatataagggagacaattgtttttttgttctttgccggcgaaccctggctctgtagggctgacctggaatttaaccagtcttccctgagccggcggaggaggacaaaaaccgccgcgaccccggcagggtgggaagtgcagggcagcgctcccaagacacgcttgttggaggttcgggcctgggtgcttggttgtctgagcctccttttttgtgtttgcctgggtcctggagaggagcgcacggtatcatggattgcagtaacggatgctccgcagagtgtaccggagaaggaggatcaaaagaggtggtggggacttttaaggctaaagacctaatagtcacaccagctaccattttaaaggaaaaaccagaccccaataatctggtttttggaactgtgttcacggatcatatgctgacggtggagtggtcctcagagtttggatgggagaaacctcatatcaagcctcttcagaacctgtcattgcaccctggctcatcagctttgcactatgcagtggaattatttgaaggattgaaggcatttcgaggagtagataataaaattcgactgtttcagccaaacctcaacatggatagaatgtatcgctctgctgtgagggcaactctgccggtatttgacaaagaagagctcttagagtgtattcaacagcttgtgaaattggatcaagaatgggtcccatattcaacatctgctagtctgtatattcgtcctacattcattggaactgagccttctcttggagtcaagaagcctaccaaagccctgctctttgtactcttgagcccagtgggaccttatttttcaagtggaacctttaatccagtgtccctgtgggccaatcccaagtatgtaagagcctggaaaggtggaactggggactgcaagatgggagggaattacggctcatctctttttgcccaatgtgaagcagtagataatgggtgtcagcaggtcctgtggctctatggagaggaccatcagatcactgaagtgggaactatgaatctttttctttactggataaatgaagatggagaagaagaactggcaactcctccactagatggcatcattcttccaggagtgacaaggcggtgcattctggacctggcacatcagtgggacacagaactcagcttgttttcaattaatttgcctgattttctgcagttcatttacttttgaacaacataattgcaattgtagactgagagaaattgaaactttcaaagagccatatttctattgcagatatattttcctgctcttccaaatctacttacagcatgagttcttcttttaaatattcaaatattttgaatattgccaagagctttgatttccatttttatctcttgtgggtttataaattaagaaaaaatactcatcttatttttttaaacctctctatttttattgccctttattcaaataacttgttgacaaactttgaacttgaaccactgaggtaaaagaacaagaattaaacagatagtttaaacacatagcttaaaaggatctttttcccatttcctatccttgagcaaagaatatattcaaacactttggcagaagtcaatgaggttataccactaattccatgatgaaaatcaactgaatgtgatactgaaagagaaggaagagaattgtcactgtaaagtcaactgttagtcatattaggaaaaaaaatacatacaatacaatttctcaaataaagtccaaatatacattcaatgtttaaaaataatgagtatttcagatatttgaactcagtctgttctttattccataaaagatataggtaagccgtgcacggtggctcacaactataatcccagcactttggcactttgggaggctgaggtgggaggatcacatgagcccagcctgggcaacatagggagaccgctatctttacaaaataaaatataaaatataaaacctagttgggcatggcagcatacacctgtagtcccaggtgctcgggagactgagacaggaggatcgcttgggcctgggaggtcgaggctgcagtgagccaagattatgccactgcattccagcctgggtgacagggcaagaccctgtcttaaaaaaaaaaaaaaaaaaaaaaaaaaaaa

SEQ ID NO: 53 - protein, 320 aa, homo sapiens
MDCSNGCSAECTGEGGSKEVVGTFKAKDLIVTPATILKEKPDPNNLVFGTVFTDHMLTVEWSSEFGWEKPHIKPLQNLSLHPGSSALHYAVELFEGLKAFRGVDNKIRLFQPNLNMDRMYRSAVRATLPVFDKEELLECIQQLVKLDQEWVPYSTSASLYIRPTFIGTEPSLGVKKPTKALLFVLLSPVGPYFSSGTFNPVSLWANPKYVRAWKGGTGDCKMGGNYGSSLFAQCEAVDNGCQQVLWLYGEDHQITEVGTMNLFLYWINEDGEEELATPPLDGIILPGVTRRCILDLAHQWDTELSLFSINLPDFLQFIYF

SEQ ID NO: 54 - DNA, 2323 bp, homo sapiens
cccgcctcctggtaggagggggtttccgcttccggcagcagcggctgcagcctcgctctggtccctgcggctggcggccgagccgtgtgtctcctcctccatcgccgccatattgtctgtgtgagcagaggggagagcggccgccgccgctgccgcttccaccacagtttgaagaaaacaggtctgaaacaaggtcttacccccagctgcttctgaacacagtgactgccagatctccaaacatcaagtccagctttgtccgccaacctgtctgacatgtcgggacccgtgccaagcagggccagagtttacacagatgttaatacacacagacctcgagaatactgggattacgagtcacatgtggtggaatggggaaatcaagatgactaccagctggttcgaaaattaggccgaggtaaatacagtgaagtatttgaagccatcaacatcacaaataatgaaaaagttgttgttaaaattctcaagccagtaaaaaagaagaaaattaagcgtgaaataaagattttggagaatttgagaggaggtcccaacatcatcacactggcagacattgtaaaagaccctgtgtcacgaacccccgccttggtttttgaacacgtaaacaacacagacttcaagcaattgtaccagacgttaacagactatgatattcgattttacatgtatgagattctgaaggccctggattattgtcacagcatgggaattatgcacagagatgtcaagccccataatgtcatgattgatcatgagcacagaaagctacgactaatagactggggtttggctgagttttatcatcctggccaagaatataatgtccgagttgcttcccgatacttcaaaggtcctgagctacttgtagactatcagatgtacgattatagtttggatatgtggagtttgggttgtatgctggcaagtatgatctttcggaaggagccatttttccatggacatgacaattatgatcagttggtgaggatagccaaggttctggggacagaagatttatatgactatattgacaaatacaacattgaattagatccacgtttcaatgatatcttgggcagacactctcgaaagcgatgggaacgctttgtccacagtgaaaatcagcaccttgtcagccctgaggccttggatttcctggacaaactgctgcgatatgaccaccagtcacggcttactgcaagagaggcaatggagcacccctatttctacactgttgtgaaggaccaggctcgaatgggttcatctagcatgccagggggcagtacgcccgtcagcagcgccaatatgatgtcagggatttcttcagtgccaaccccttcaccccttggacctctggcaggctcaccagtgattgctgctgccaacccccttgggatgcctgttccagctgccgctggcgctcagcagtaacggccctatctgtctcctgatgcctgagcagaggtgggggagtccaccctctccttgatgcagcttgcgcctggcggggaggggtgaaacacttcagaagcaccgtgtctgaaccgttgcttgtggatttatagtagttcagtcataaaaaaaaaattataataggctgattttcttttttctttttttttttaactcgaacttttcataactcaggggattccctgaaaaattacctgcaggtggaatatttcatggacaaatttttttttctcccctcccaaatttagttcctcatcacaaaagaacaaagataaaccagcctcaatcccggctgctgcatttaggtggagacttcttcccattcccaccattgttcctccaccgtcccacactttagggggttggtatctcgtgctcttctccagagattacaaaaatgtagcttctcaggggaggcaggaagaaaggaaggaaggaaagaaggaagggaggacccaatctataggagcagtggactgcttgctggtcgcttacatcactttactccataagcgcttcagtggggttatcctagtggctcttgtggaagtgtgtcttagttacatcaagatgttgaaaatctacccaaaatgcagacagatactaaaaacttctgttcagtaagaatcatgtcttactgatctaaccctaaatccaactcatttatacttttatttttagttcagtttaaaatgttgataccttccctcccaggctccttaccttggtcttttccctgttcatctcccaacatgctgtgctccatagctggtaggagagggaaggcaaaatctttcttagttttctttgtcttggccattttgaattc

SEQ ID NO: 55 - protein, 391 aa, homo sapiens
MSGPVPSRARVYTDVNTHRPREYWDYESHVVEWGNQDDYQLVRKLGRGKYSEVFEAINITNNEKVVVKILKPVKKKKIKREIKILENLRGGPNIITLADIVKDPVSRTPALVFEHVNNTDFKQLYQTLTDYDIRFYMYEILKALDYCHSMGIMHRDVKPHNVMIDHEHRKLRLIDWGLAEFYHPGQEYNVRVASRYFKGPELLVDYQMYDYSLDMWSLGCMLASMIFRKEPFFHGHDNYDQLVRIAKVLGTEDLYDYIDKYNIELDPRFNDILGRHSRKRWERFVHSENQHLVSPEALDFLDKLLRYDHQSRLTAREAMEHPYFYTVVKDQARMGSSSMPGGSTPVSSANMMSGISSVPTPSPLGPLAGSPVIAAANPLGMPVPAAAGAQQ

SEQ ID NO: 56 - DNA, 2502 bp, homo sapiens
ctctttggccaagccctgcctctgtacagcctcgagtggacagccagaggctgcagctggagcccagagcccaagatggagccccagctggggcctgaggctgccgccctccgccctggctggctggccctgctgctgtgggtctcagccctgagctgttctttctccttgccagcttcttccctttcttctctggtgccccaagtcagaaccagctacaattttggaaggactttcctcggtcttgataaatgcaatgcctgcatcgggacatctatttgcaagaagttctttaaagaagaaataagatctgacaactggctggcttcccaccttggactgcctcccgattccttgctttcttatcctgcaaattactcagatgattccaaaatctggcgccctgtggagatctttagactggtcagcaaatatcaaaacgagatctcagacaggaaaatctgtgcctctgcatcagccccaaagacctgcagcattgagcgtgtcctgcggaaaacagagaggttccagaaatggctgcaggccaagcgcctcacgccggacctggtgcaggactgtcaccagggccagagagaactaaagttcctgtgtatgctgagataacaccagtgaaaaagcctggcatggagcccagcactgagaacttccagaaagtgttagccttctcccaactgtgttataccaaccacattttcaaatagtaatcattaaagaggcttctgcatcaaaccttcacatgcagctcccatgccaccctccagaattcaccaacacacaggcccaccagcaacaggctacctttgcacaatattctctgatgacaactccaaagccccggctctttccaccacactgtggtcccctagatggggctgttgctgagcccaccccaatccagatgtgatccccctgtgatctacttctggcaagattctcagtctggacaggtcttccctatgagatagaacctgataaggagctagggcaattctgacaacattaccaaaggcccacataacttctaaattttggtctggtctgaaggaaaacctgttctcgccctagtgatggatgaactctcttatctctggcttctagagggaaaaaaaaagcatacctcttttactttttaagtacctccatcagagtcatgaaatcacctgtcaagactatctatcttttatgtttccattctggtaagaactctttaaatgaggacactgctgattgctggtgatgttttttgagcaaacactcgggggtatggatgaaagccaatcgcaggtcaaatgactccttggggaagctacttctcctctattcagatttcactaaaatcttccaagatgaaagcaaatctagatttcggtcttcattgctgtccatttttgtaatgaacgagtgtttttcctttagctagtgtatcaggcagggttctaccagagaaacagaaccagtaggagatacatatacatgtccagatttatttcaaagaattgatttacatgattgtggggattggcaagtccaaaatccatatggtaggcctgcaatctgtaaacctttgggcaggagctgatgctgtagtttgcagatagaattccttgttccttaaaaaaatctgtttttgttcttaagggctttgaatgattggatcaggcccacccagattacctagataatctcttttacttaaagtaaactgattgtaggtgctaatcacatctatgaaatgccttcacagcaacacctagattagcattcaattgaataactggggaatacagcctagccaagttgacacataaaattaaccatcacagcaacatgcctgctaaattttatcgaccgtcttcagactgttaaggattgtggtagagaactgtgacagccactctcagcatcaccctgaaccaaaggcccctatcaagtaacaatatagccaagcaaaattccagtcaatagagacattgactggttggctggcttcccaagggatagcaccagacaagaaatgcaaggatgaggaaaccaggcacgggagagggaggggcaacagaggtccagggtttggttatctttttatttttcactgggaggtggtaagttagccctgttgcccatgtatgcagatgggagaagtgatttagaaactccaaagcaattggtaatccccaaaatgggtgtatctggtttgaaatgaaaccttattttattggaaatggttggtttcccaattctgtttgccattggccaatataattgtgggtttgcacatggccagcacatgccaaacagaagtagacaaaggtctcactctgtaagtgggaccttggggaggagctgcctccatcataaagggaggggttagtaaaaatggtctcttaagcctgttcctgctacagttatagaggttgctcagaaccttctcagcaaatatagcagttatctattgttgtgtattaaaccatttcaacacat

SEQ ID NO: 57 - protein, 182 aa, homo sapiens
MEPQLGPEAAALRPGWLALLLWVSALSCSFSLPASSLSSLVPQVRTSYNFGRTFLGLDKCNACIGTSICKKFFKEEIRSDNWLASHLGLPPDSLLSYPANYSDDSKIWRPVEIFRLVSKYQNEISDRKICASASAPKTCSIERVLRKTERFQKWLQAKRLTPDLVQDCHQGQRELKFLCMLR

SEQ ID NO: 58 - DNA, 3057 bp, homo sapiens
actttgctacggagtgcatcggacgtcgaagcctagagtctctgcgtctttccctcttccgctgcctcattcctttccttcctagccttggtcgtcgccgccaccatgaacaagaagaagaaaccgttcctagggatgcccgcgcccctcggctacgtgccggggctgggccggggcgccactggcttcaccacgcggtcagacattgggcccgcccgtgatgcaaatgaccctgtggatgatcgccatgcacccccaggcaagagaaccgttggggaccagatgaagaaaaatcaggctgctgacgatgacgacgaggatctaaatgacaccaattacgatgagtttaatggctatgctgggagcctcttctcaagtggaccctacgagaaagatgatgaggaagcagatgctatctatgcagccctggataaaaggatggatgaaagaagaaaagaaagacgggagcaaagggagaaagaagaaatagagaaatatcgtatggaacgccccaaaatccaacagcagttctcagacctcaagaggaagttggcagaagtcacagaagaagagtggctgagcatccccgaggttggcgatgccagaaataaacgtcagcggaacccacgctatgagaagctgacccctgttcctgacagtttctttgccaaacatttacagaccggagagaaccatacctcagtggatccccgacaaactcaatttggaggtcttaacacaccctatccaggtggactaaacactccatacccaggtggaatgacgccaggactgatgacacctggcacaggtgagctggacatgaggaagattggccaagcgaggaacactctgatggacatgaggctgagccaggtgtctgactccgtgagtggacagaccgtcgttgaccccaaaggctacctgacggatttaaattccatgatcccgacacacggaggagacatcaatgatatcaagaaggcgcgactgctcctcaagtctgttcgggagacgaaccctcatcacccgccagcctggattgcatcagcccgcctggaagaagtcactgggaagctacaagtagctcggaaccttatcatgaaggggacggagatgtgccccaagagtgaagatgtctggctggaagcagccaggttgcagcctggggacacagccaaggccgtggtagcccaagctgtccgtcatctcccacagtctgtcaggatttacatcagagccgcagagctggaaacggacattcgtgcaaagaagcgggttcttcggaaagccctcgagcatgttccaaactcggttcgcttgtggaaagcagccgttgagctggaagaacctgaagatgctagaatcatgctgagccgagctgtggagtgctgccccaccagcgtggagctctggcttgctctggcaaggctggagacctatgaaaatgcccgcaaggtcttgaacaaggcgcgggagaacattcctacagaccgacatatctggatcacggctgctaagctggaggaagccaatgggaacacgcagatggtggagaagatcatcgaccgagccatcacctcgctgcgggccaacggtgtggagatcaaccgtgagcagtggatccaggatgccgaggaatgtgacagggctgggagtgtggccacctgccaggccgtcatgcgtgccgtgattgggattgggattgaggaggaagatcggaagcatacctggatggaggatgctgacagttgtgtagcccacaatgccctggagtgtgcacgagccatctacgcctacgccctgcaggtgttccccagcaagaagagtgtgtggctgcgcgccgcgtacttcgagaagaaccatggcactcgggagtccctggaagcactcctgcagagggctgtggcccactgccccaaagcagaggtgctgtggctcatgggcgccaagtccaagtggctggcaggggatgtgcctgcagcaaggagcatcctggccctggccttccaggccaaccccaacagtgaggagatctggctggcagccgtgaagctggagtccgagaatgatgagtacgagcgggcccggaggctgctggccaaggcgcggagcagtgcccccaccgcccgggtgttcatgaagtctgtgaagctggagtgggtgcaagacaacatcagggcagcccaagatctgtgcgaggaggccctgcggcactatgaggacttccccaagctgtggatgatgaaggggcagatcgaggagcagaaggagatgatggagaaggcgcgggaagcctataaccaggggttgaagaagtgtccccactccacacccctgtggcttttgctctctcggctggaggagaagattgggcagcttactcgagcacgggccattttggaaaagtctcgtctgaagaacccaaagaaccctgggctgtggttggagtccgtgcggctggagtaccgtgcggggctgaagaacatcgcaaatacactcatggccaaggcgctgcaggagtgccccaactccggtatcctgtggtctgaggccatcttcctcgaggcaaggccccagaggaggaccaagagcgtggatgccctgaagaagtgtgagcatgacccccatgtgctcctggccgtggccaagctgttttggagtcagcggaagatcaccaaggccagggagtggttccaccgcactgtgaagattgactcggacctgggggatgcctgggccttcttctacaagtttgagctgcagcatggcactgaggagcagcaggaggaggtgaggaagcgctgtgagagtgcagagcctcggcatggggagctgtggtgcgccgtgtccaaggacatcgccaactggcagaagaagatcggggacatccttaggctggtggccggccgcatcaagaacaccttctgattgagcggttgccatggccggtctccgtggggcagggttgggccgcatgtggaagggctctgagctgtgtcctccttcattaaaagtttttatgtctcgtgtcagaaaaaaaaaaaaaaaaaaaaa

SEQ ID NO: 59 - protein, 941 aa, homo sapiens
MNKKKKPFLGMPAPLGYVPGLGRGATGFTTRSDIGPARDANDPVDDRHAPPGKRTVGDQMKKNQAADDDDEDLNDTNYDEFNGYAGSLFSSGPYEKDDEEADAIYAALDKRMDERRKERREQREKEEIEKYRMERPKIQQQFSDLKRKLAEVTEEEWLSIPEVGDARNKRQRNPRYEKLTPVPDSFFAKHLQTGENHTSVDPRQTQFGGLNTPYPGGLNTPYPGGMTPGLMTPGTGELDMRKIGQARNTLMDMRLSQVSDSVSGQTVVDPKGYLTDLNSMIPTHGGDINDIKKARLLLKSVRETNPHHPPAWIASARLEEVTGKLQVARNLIMKGTEMCPKSEDVWLEAARLQPGDTAKAVVAQAVRHLPQSVRIYIRAAELETDIRAKKRVLRKALEHVPNSVRLWKAAVELEEPEDARIMLSRAVECCPTSVELWLALARLETYENARKVLNKARENIPTDRHIWITAAKLEEANGNTQMVEKIIDRAITSLRANGVEINREQWIQDAEECDRAGSVATCQAVMRAVIGIGIEEEDRKHTWMEDADSCVAHNALECARAIYAYALQVFPSKKSVWLRAAYFEKNHGTRESLEALLQRAVAHCPKAEVLWLMGAKSKWLAGDVPAARSILALAFQANPNSEEIWLAAVKLESENDEYERARRLLAKARSSAPTARVFMKSVKLEWVQDNIRAAQDLCEEALRHYEDFPKLWMMKGQIEEQKEMMEKAREAYNQGLKKCPHSTPLWLLLSRLEEKIGQLTRARAILEKSRLKNPKNPGLWLESVRLEYRAGLKNIANTLMAKALQECPNSGILWSEAIFLEARPQRRTKSVDALKKCEHDPHVLLAVAKLFWSQRKITKAREWFHRTVKIDSDLGDAWAFFYKFELQHGTEEQQEEVRKRCESAEPRHGELWCAVSKDIANWQKKIGDILRLVAGRIKNTF

SEQ ID NO: 60 - DNA, 1287 bp, homo sapiens
ccctttcgctgcggcctttccccaacccggacccggcacttctcgggttccgcgactgccgatcgccccggcgcggcaccgctccctcaggagtcgcctaggccgcgcagtctcccgacttctcgtcaggctttcgcgccggcgctccagcaatcactggctggagaaggtgggcgttccggctcgagaggaccctgccgcggctccggaagagcctcgtcctgggcggcggtggtgcggcggtcgccgttatggccactgggctgggcggctgaccgcgggctaggaaagggcccagggcccgaatctcggtggccgctgctccagcgcggcctgcgccatggcctcctccgccgcctcctcggagcatttcgagaagctgcacgagatcttccgcggcctccatgaagacctacaaggggtgcccgagcggctgctggggacggcggggaccgaagaaaagaagaaattgatcagggattttgatgaaaagcaacaggaagcaaatgaaacgctggcagagatggaggaggagctacgttatgcacccctgtctttccgaaaccccatgatgtctaagcttcgaaactaccggaaggaccttgctaaactccatcgggaggtgagaagcacacctttgacagccacacctggaggccgaggagacatgaaatatggcatatatgctgtagagaatgagcatatgaatcggctacagtctcaaagggcaatgcttctgcagggcactgaaagcctgaaccgggccacccaaagtattgaacgttctcatcggattgccacagagactgaccagattggctcagaaatcatagaagagctgggggaacaacgagaccagttagaacgtaccaagagtagactggtaaacacaagtgaaaacttgagcaaaagtcggaagattctccgttcaatgtccagaaaagtgacaaccaacaagctgctgctttccattatcatcttactggagctcgccatcctgggaggcctggtttactacaaattctttcgcagccattgaacttctatagggaagggtttgtggaccagaactttgaccttgtgaatgcatgatgttagggatgtggatagaataagcatattgctgctgtgggctgacagttcaaggatgcactgtatagccaggctgtgggaggagggaggaaagatgaaaaaccacttaaatgtgaaggaacaacagcaacaagaccagtatgatataccaaggtaataaatgctgtttatgacttctttaaaaaaaaaaaaaaa

SEQ ID NO: 61 - protein, 232 aa, homo sapiens
MASSAASSEHFEKLHEIFRGLHEDLQGVPERLLGTAGTEEKKKLIRDFDEKQQEANETLAEMEEELRYAPLSFRNPMMSKLRNYRKDLAKLHREVRSTPLTATPGGRGDMKYGIYAVENEHMNRLQSQRAMLLQGTESLNRATQSIERSHRIATETDQIGSEIIEELGEQRDQLERTKSRLVNTSENLSKSRKILRSMSRKVTTNKLLLSIIILLELAILGGLVYYKFFRSH

SEQ ID NO: 62 - DNA, 1869 bp, homo sapiens
ccgagggtcaggctgcagaagcccagaatcccaccccagtccccaagtacagaggtcgctgtcaagatggagtttccaacccagtaaatccaagggccagaccgtgacctcataaagcatgatctccttctgtccagactgtggcaaaagtatccaagcggcattcaaattctgcccctactgtggaaattctttgcctgtagaggagcatgtagggtcccagacctttgtcaatccacatgtgtcatccttccaaggctcaaagagagggctgaactccagttttgaaacctctcctaagaaagtgaaatggtccagcaccgtcacctctccccgattatccctcttctcagatggtgacagttctgagtctgaagatactctgagttcctctgagagatccaaaggctccgggagcagacccccaacccccaaaagcagccctcagaagaccaggaagagccctcaggtgaccaggggtagccctcagaagaccagctgtagccctcagaagaccaggcagagccctcagacgctgaagcggagccgagtgaccacctcacttgaagctttgcccacagggacagtgctgacagacaagagtgggcgacagtggaagctgaagtccttccagaccagggacaaccagggcattctctatgaagctgcacccacctccaccctcacctgtgactcaggaccacagaagcaaaagttctcactcaaactggatgccaaggatgggcgcttgttcaatgagcagaacttcttccagcgggccgccaagcctctgcaagtcaacaagtggaagaagctgtactcgaccccactgctggccatccctacctgcatgggtttcggtgttcaccaggacaaatacaggttcttggtgttacccagcctggggaggagccttcagtcggccctggatgtcagcccaaagcatgtgctgtcagagaggtctgtgctgcaggtggcctgccggctgctggatgccctggagttcctccatgagaatgagtatgttcatggaaatgtgacagctgaaaatatctttgtggatccagaggaccagagtcaggtgactttggcaggctatggcttcgccttccgctattgcccaagtggcaaacacgtggcctacgtggaaggcagcaggagccctcacgagggggaccttgagttcattagcatggacctgcacaagggatgcgggccctcccgccgcagcgacctccagagcctgggctactgcatgctgaagtggctctacgggtttctgccatggacaaattgccttcccaacactgaggacatcatgaagcaaaaacagaagtttgttgataagccggggcccttcgtgggaccctgcggtcactggatcaggccctcagagaccctgcagaagtacctgaaggtggtgatggccctcacgtatgaggagaagccgccctacgccatgctgaggaacaacctagaagctttgctgcaggatctgcgtgtgtctccatatgaccccattggcctcccgatggtgccctaggtggaatccagaactttccatttgcagtgtgcaacagaaaaaaaaatgaagcaatgtgactcaaggcctgctgtttaatcacagataagcttctagaacaagccctggaatgtgcattcctgccactggtttcaggatactcatcagtcctgattagcctccggagggccccagtttccctcccgtgaatgtgaagttccccatcttggtggcctgcccttcagccagtgtcctagcaaagctggatggggttgggccggcccacaggggggacccctcctacccttgactcctctgtgctttggtaataaattgttttaccagag

SEQ ID NO: 63 - protein, 474 aa, homo sapiens
MISFCPDCGKSIQAAFKFCPYCGNSLPVEEHVGSQTFVNPHVSSFQGSKRGLNSSFETSPKKVKWSSTVTSPRLSLFSDGDSSESEDTLSSSERSKGSGSRPPTPKSSPQKTRKSPQVTRGSPQKTSCSPQKTRQSPQTLKRSRVTTSLEALPTGTVLTDKSGRQWKLKSFQTRDNQGILYEAAPTSTLTCDSGPQKQKFSLKLDAKDGRLFNEQNFFQRAAKPLQVNKWKKLYSTPLLAIPTCMGFGVHQDKYRFLVLPSLGRSLQSALDVSPKHVLSERSVLQVACRLLDALEFLHENEYVHGNVTAENIFVDPEDQSQVTLAGYGFAFRYCPSGKHVAYVEGSRSPHEGDLEFISMDLHKGCGPSRRSDLQSLGYCMLKWLYGFLPWTNCLPNTEDIMKQKQKFVDKPGPFVGPCGHWIRPSETLQKYLKVVMALTYEEKPPYAMLRNNLEALLQDLRVSPYDPIGLPMVP

SEQ ID NO: 64 - DNA, 1543 bp, homo sapiens
cgcccccgccgggcgtgtgtgtcgtgtgtgtttggggcccgcgcgggttgcgcgccctccgccttcgcgcctcctgcccccgaggccctactgctgcccctgtgcccctcgccccgccgggcgtcgcgggccaacatgggccaggaagaggagctgctgaggatcgccaaaaagctggagaagatggtggccaggaagaacacggaaggggccctggaccttctgaagaagctgcacagctgccagatgtccatccagctactacagacaaccaggattggagttgctgttaatggggtccgcaagcactgctcagacaaggaggtggtgtccttggccaaagtccttatcaaaaactggaagcggctgctagactcccctggacccccaaaaggagaaaaaggagaggaaagagaaaaggcaaagaagaaggaaaaagggcttgagtgttcagactggaagccagaagcaggcctttctccaccaaggaaaaaacgagaagaccccaaaaccaggagagactctgtggactccaagtcttctgcctcctcctctccaaaaagaccatcggtggaaagatcaaacagcagcaaatcaaaagcggagagccccaaaacacctagcagccccttgacccccacgtttgcctcttccatgtgtctcctggccccctgctatctcacaggggactctgtccgggacaagtgtgtggagatgctgtcagcagccctgaaggcggacgatgattacaaggactatggagtcaactgtgacaagatggcatcagaaatcgaagatcatatctaccaagagctcaagagcacggacatgaagtaccggaaccgcgtgcgcagccgcataagcaacctcaaggaccccaggaaccccggcctgcggcggaacgtgctcagtggggccatctccgcagggcttatagccaagatgacggcagaggaaatggccagtgatgaactgagggagttgaggaatgccatgacccaggaggccatccgtgagcaccagatggccaagactggcggcaccaccactgacctcttccagtgcagcaaatgcaagaagaagaactgcacctataaccaggtgcagacacgcagtgctgatgagcccatgactacctttgtcttatgcaatgaatgtggcaatcgctggaagttctgctgatggaacagccagccatgaacaaggtgaggaagaagaaagaggaagcgctgaattatctgaactggagaagcaataaaaattaaagtgaaggaaaatactgaactctgtctgagtgggatggtatgagttagaggaagaattctcttgcaaattaataatcggtcattagaaacaattggttaatgggggagcctaattggagaatgatgctgagaatttgtattgatgaacctcttttagaaactgcagagggctgggcacggtggtttatggctgtaatctgcaaactctgggaggctgaggtgggagaatcgcttaaccccagaagtttgagtccagcccaggcaacacagcaagaccc

SEQ ID NO: 65 - protein, 348 aa, homo sapiens
MGQEEELLRIAKKLEKMVARKNTEGALDLLKKLHSCQMSIQLLQTTRIGVAVNGVRKHCSDKEVVSLAKVLIKNWKRLLDSPGPPKGEKGEEREKAKKKEKGLECSDWKPEAGLSPPRKKREDPKTRRDSVDSKSSASSSPKRPSVERSNSSKSKAESPKTPSSPLTPTFASSMCLLAPCYLTGDSVRDKCVEMLSAALKADDDYKDYGVNCDKMASEIEDHIYQELKSTDMKYRNRVRSRISNLKDPRNPGLRRNVLSGAISAGLIAKMTAEEMASDELRELRNAMTQEAIREHQMAKTGGTTTDLFQCSKCKKKNCTYNQVQTRSADEPMTTFVLCNECGNRWKFC

SEQ ID NO: 66 - DNA, 2430 bp, homo sapiens
accggcagcgaggcgccgctcccgccgcctcagcccggccttcctcggctccggcgctccggtcgcggggcccgggttcctcggcacaccccgctccagccgcccccagagcctgtccccagcccttcggaagccccggcgccagcccgggccctcggcagggaggatgacggagctgcagtcggcactgctactgcgaagacagctggcagaactcaacaaaaatccagtggaaggcttttctgcaggtttaatagatgacaatgatctctaccgatgggaagtccttattattggccctccagatacactttatgaaggtggtgtttttaaggctcatcttactttcccaaaagattatcccctccgacctcctaaaatgaaattcattacagaaatctggcacccaaatgttgataaaaatggtgatgtgtgcatttctattcttcatgagcctggggaagataagtatggttatgaaaagccagaggaacgctggctccctatccacactgtggaaaccatcatgattagtgtcatttctatgctggcagaccctaatggagactcacctgctaatgttgatgctgcgaaagaatggagggaagatagaaatggagaatttaaaagaaaagttgcccgctgtgtaagaaaaagccaagagactgcttttgagtgacatttatttagcagctagtaacttcacttatttcagggtctccaattgagaaacatggcactgtttttcctgcactctacccacctattgctggacttctgttgtacaagttggcaaacactggctggaactgggctgcaataaaacatgccagttatcaatgctgacaagagcctaacaagtgccaacttacagatgattacgcattttgaattctaatgaactgttttaaccttcaggaagaattgtaaagacctgtacatagcacaacatgatccggataatatatatactgttcatgtacatccacaaatacaccttgtaccaaataatgctttcttgtagtagaataagaatcgtgtaaattctaagagattttagcaggttttctttcctattcattgtttcttatcagtttaaaaggattcctttaagcatgtcagatgaaaagcaattaggattaaaagtttccatttaatttcccttaaacccttgaggcttcattaaactcttttcacttactaaacttttgtatcttctttgttttgacacactcccctttgcttttatctcttacctgccagaatgttctcaaatgatttagttcaaatactgaaatacttaatgagcaattacttgatttttaatgatgacttcgaaggagtcatcactaggtgctttgtcctttttgtattctagttgcacccacctcttggattggatatagcaataacatttattggccgttgtgagctcttgatcccagtcattacccctgagaactaaaaatagatggttcttaattcaacttactgaaaatttccccaaacaatagcaaatctgacttttccctcttcagttgcctggtattaaggttggataaatgaagcatgcacagctacaggctttctacttaacttctgggtttgctattacaaatcctatttactctcatacccttctccttagtccttcatatttctctgcctctattcttctatactgcagatttttctcacctattgtacaaagaaattgcgatgtatattttcatgtaatttgattttggaattctgtcaccttatgtagtgagttcttccaaaatataattttttttcaataattgtcaagttgttggcttttattgtattgaatgaaggctataatactgagtgccagagaagtggtttaggaaaatctcaggttgattccttatgcaaatgaacttttaatacttgaaaatcacatggccatggcagtatatgtatttggttctatctagattcttctgtgaatctaaaagcattacaggggtaaatgctttgctatttgacgtatagatcccgtcactaacaatagtacacttggatgtgattaatgtttgagcttcaatatatttcatatcatacagttttctaaaacaacttcagcaaatggtaaaatgaacatgtgcagtgttaaaggcaggccttaggctccttcatgtttgttgtgaggttgtgtgtgggaagtagtctttggcttataagggatagaacttgagacagtagcagatgggacatggtgtttgattgtgagaatcagtgagaattcgtgcatctctgctctgtggggtttggagaaatgctttggcagaagagtgaaagaactcctgccaagagcccagacctctacaaacgttgtatgtccttttttaagcagaaataaaatggttgaggacgaaaaaaaaaaaaaaaaaaaaaaaaaaaaaa

SEQ ID NO: 67 - protein, 170 aa, homo sapiens
MTELQSALLLRRQLAELNKNPVEGFSAGLIDDNDLYRWEVLIIGPPDTLYEGGVFKAHLTFPKDYPLRPPKMKFITEIWHPNVDKNGDVCISILHEPGEDKYGYEKPEERWLPIHTVETIMISVISMLADPNGDSPANVDAAKEWREDRNGEFKRKVARCVRKSQETAFE

SEQ ID NO: 68 - DNA, 2258 bp, homo sapiens
gaagcttgaggccggagacgcccgccttcgggcccgtccgcccggcttccccgctcccgggtactggaagatgaaagagactatacaagggaccgggtcctgggggcctgagcctcctggacccggcatacccccagcttactcaagtcccaggcgggagcgtcttcgttggcccccacctcccaaaccccgactcaagtcaggtggagggtttgggccagatcctgggtcagggaccacagtgccagccagacgcctccctgtcccccgaccctcttttgatgcctcagcaagtgaagaggaggaagaagaggaggaggaggaggatgaagatgaagaggaggaagtggcagcttggaggctgcccccaagatggagtcagctgggaacctcccagcggccccgcccttcccgccccactcatcgaaaaacctgctcacagcgccgccgccgagccatgagagccttccggatgctgctctactcaaaaagcacctcgctgacattccactggaagctttgggggcgccaccggggccggcggcggggcctcgcacaccccaagaaccatctttcaccccagcaagggggtgcgacgccacaggtgccatccccctgttgtcgttttgactccccccgggggccacctccaccccggctgggtctgctaggtgctctcatggctgaggatggggtgagagggtctccaccagtgccctctgggccccccatggaggaagatggactcaggtggactccaaagtctcctctggaccctgactcgggcctcctttcatgtactctgcccaacggttttgggggacaatctgggccagaaggggagcgcagcttggcaccccctgatgccagcatcctcatcagcaatgtgtgcagcatcggggaccatgtggcccaggagctttttcagggctcagatttgggcatggcagaagaggcagagaggcctggggagaaagccggccagcacagccccctgcgagaggagcatgtgacctgcgtacagagcatcttggacgaattccttcaaacgtatggcagcctcatacccctcagcactgatgaggtagtagagaagctggaggacattttccagcaggagttttccaccccttccaggaagggcctggtgttgcagctgatccagtcttaccagcggatgccaggcaatgccatggtgaggggcttccgagtggcttataagcggcacgtgctgaccatggatgacttggggaccttgtatggacagaactggctcaatgaccaggtgatgaacatgtatggagacctggtcatggacacagtccctgaaaaggtgcatttcttcaatagtttcttctatgataaactccgtaccaagggttatgatggggtgaaaaggtggaccaaaaacgtggacatcttcaataaggagctactgctaatccccatccacctggaggtgcattggtccctcatctctgttgatgtgaggcgacgcaccatcacctattttgactcgcagcgtaccctaaaccgccgctgccctaagcatattgccaagtatctacaggcagaggcggtaaagaaagaccgactggatttccaccagggctggaaaggttacttcaaaatgaatgtggccaggcagaataatgacagtgactgtggtgcttttgtgttgcagtactgcaagcatctggccctgtctcagccattcagcttcacccagcaggacatgcccaaacttcgtcggcagatctacaaggagctgtgtcactgcaaactcactgtgtgagcctcgtaccccagaccccaagcccataaatgggaagggagacatgggagtcccttcccaagaaactccagttcctttcctctcttgcctcttcccactcacttccctttggtttttcatatttaaatgtttcaatttctgtatttttttttctttgagagaatacttgttgatttctgatgtgcagggggtggctacagaaaagcccctttcttcctctgtttgcaggggagtgtggccctgtggcctgggtggagcagtcatcctcccccttccccgtgcagggagcaggaaatcagtgctgggggtggtgggcggacaataggatcactgcctgccagatcttcaaacttttatatatatatatatatatatatatatatatataaaaatatataaatgccacggtcctgctctggtcaataaaggatcctttgttgatacgtaaaaaaaaaaaaaaaaa

SEQ ID NO: 69 - protein, 574 aa, homo sapiens
MKETIQGTGSWGPEPPGPGIPPAYSSPRRERLRWPPPPKPRLKSGGGFGPDPGSGTTVPARRLPVPRPSFDASASEEEEEEEEEEDEDEEEEVAAWRLPPRWSQLGTSQRPRPSRPTHRKTCSQRRRRAMRAFRMLLYSKSTSLTFHWKLWGRHRGRRRGLAHPKNHLSPQQGGATPQVPSPCCRFDSPRGPPPPRLGLLGALMAEDGVRGSPPVPSGPPMEEDGLRWTPKSPLDPDSGLLSCTLPNGFGGQSGPEGERSLAPPDASILISNVCSIGDHVAQELFQGSDLGMAEEAERPGEKAGQHSPLREEHVTCVQSILDEFLQTYGSLIPLSTDEVVEKLEDIFQQEFSTPSRKGLVLQLIQSYQRMPGNAMVRGFRVAYKRHVLTMDDLGTLYGQNWLNDQVMNMYGDLVMDTVPEKVHFFNSFFYDKLRTKGYDGVKRWTKNVDIFNKELLLIPIHLEVHWSLISVDVRRRTITYFDSQRTLNRRCPKHIAKYLQAEAVKKDRLDFHQGWKGYFKMNVARQNNDSDCGAFVLQYCKHLALSQPFSFTQQDMPKLRRQIYKELCHCKLTV

SEQ ID NO: 70 - DNA, 2733 bp, homo sapiens
aagggaagatggagacaatactggagcagcagcggcgctatcatgaggagaaggaacggctcatggacgtcatggctaaagagatgctcaccaagaagtccacgctccgggaccagatcaattctgatcaccgcactcgggccatgcaagataggtatatggaggtcagtgggaacctgagggatttgtatgatgataaggatggattacgaaaggaggagctcaatgccatttcaggacccaatgagtttgctgaattctataatagactcaagcaaataaaggaattccaccggaagcacccaaatgagatctgtgtgccaatgtcagtggaatttgaggaactcctgaaggctcgagagaatccaagtgaagaggcacaaaacttggtggagttcacagatgaggagggatatggtcgttatctcgatctccatgactgttacctcaagtacattaacctgaaggcatctgagaagctggattatatcacatacctgtccatctttgaccaattatttgacattcctaaagaaaggaagaatgcagagtataagagatacctagagatgctgcttgagtaccttcaggattacacagatagagtgaagcctctccaagatcagaatgaactttttgggaagattcaggctgagtttgagaagaaatgggagaatgggacctttcctggatggccgaaagagacaagcagtgccctgacccatgctggagcccatcttgacctctctgcattctcctcctgggaggagttggcttctctgggtttggacagattgaaatctgctctcttagctttaggcttgaaatgtggcgggaccctagaagagcgagcccagagactattcagtaccaaaggaaagtccctggagtcacttgatacctctttgtttgccaaaaatcccaagtcaaagggcaccaagcgagacactgaaaggaacaaagacattgcttttctagaagcccagatctatgaatatgtagagattctcggggaacagcgacatctcactcatgaaaatgtacagcgcaagcaagccaggacaggagaagagcgagaagaagaggaagaagagcagatcagtgagagtgagagtgaagatgaagagaacgagatcatttacaaccccaaaaacctgccacttggctgggatggcaaacctattccctactggctgtataagcttcatggcctaaatatcaactacaactgtgagatttgtggaaactacacctaccgagggcccaaagccttccagcgacactttgctgaatggcgtcatgctcatggcatgaggtgtttgggcatcccaaatactgctcactttgctaatgtgacacagattgaagatgctgtctccttgtgggccaaactgaaattgcagaaggcttcagaacgatggcagcctgacactgaggaagaatatgaagactcaagtgggaatgttgtgaataagaagacatacgaggatctgaaaagacaaggactgctctagtgttgagggatgtagctcagcttttgggctagcccaggcttccctaagatctgctttttctatttctcccaaccaaatcctcttaaagaccctttgctatgtagtctcatggtctagcatgcatcttgtagaaacaaggcatgctggcagattgcagggttgagatgtgttttatctgttttatattttaaaagattctgccagaaaataaaaccagaccttgttctaaagcccagggttatggaccaactcagtgcttcaggtcttaatgcctccatacctcttcctcaccaactttactagtagctgagatttaatgggcacctattatgctacatatcatgttaggtaaatctgacctgacctctttccccaccctcctttgttgctgcttccctgaatgagtattaccccaggatgaggtctgccatcagcttagttagccattgatgcaaatactagggaaagactaggaggatgagccagggttgctactaaggactaagtgtcgcaccaaggtttgccttttgtatttgcataaagaaaggagttggagctgggtgcagtggcttgtgcctgtagtcccagctacttgggaggctgaggcaggagggttgcttgagactagcctaggtaacatagtgagaccctgtctcattaaaaaaaaaaaaaaaaggcatggtggcacgcactgtagtcccagctactcaggagactgaggctagaagatcctttgaacctaggagtttgagaccagcctgggcgatatagtgaggccccatctcaaaaaaaaaaaaaagcggggggggggagttgggctgtgttggaatgggcctgcagcccaacaaacaagggaactaggaccgacagtgacttcaccagcttgctaggtcagaatgagagactggtgggtctgtctacctgtttcttctacaagatccctatttgactgtaaaagtagctaatactcacatgttctccaatcccaggtagccatggtagagttgggtagagttgagcagccgccccaggatccaaatgtggtgtctgaaatggaaagaactaaggcaaccaggaaggcactgatctgccttataagcacagtcatctgaaagtcaggcctgctgcaggacaggatcccccagagaccccatttgcctctcaacactcagaccttcaactgttttttaataaatctactttttaaaaaaaaaaaata

SEQ ID NO: 71 - protein, 501 aa, homo sapiens
METILEQQRRYHEEKERLMDVMAKEMLTKKSTLRDQINSDHRTRAMQDRYMEVSGNLRDLYDDKDGLRKEELNAISGPNEFAEFYNRLKQIKEFHRKHPNEICVPMSVEFEELLKARENPSEEAQNLVEFTDEEGYGRYLDLHDCYLKYINLKASEKLDYITYLSIFDQLFDIPKERKNAEYKRYLEMLLEYLQDYTDRVKPLQDQNELFGKIQAEFEKKWENGTFPGWPKETSSALTHAGAHLDLSAFSSWEELASLGLDRLKSALLALGLKCGGTLEERAQRLFSTKGKSLESLDTSLFAKNPKSKGTKRDTERNKDIAFLEAQIYEYVEILGEQRHLTHENVQRKQARTGEEREEEEEEQISESESEDEENEIIYNPKNLPLGWDGKPIPYWLYKLHGLNINYNCEICGNYTYRGPKAFQRHFAEWRHAHGMRCLGIPNTAHFANVTQIEDAVSLWAKLKLQKASERWQPDTEEEYEDSSGNVVNKKTYEDLKRQGLL

SEQ ID NO: 72 - DNA, 2514 bp, homo sapiens
gaggctgcgaggagccggcgcggtcgcagtctccacggcgcaggcccacggtagcgcagccgctctgagtagaacttcatggaggaacacggagtgacccaaaccgaacatatggctaccatagaagcacatgcagtggcccagcaagtgcagcaggtccatgtggctacttacaccgagcatagtatgctgagtgctgatgaagactcgccttcttctcccgaggacacctcttacgatgactcagatatactcaactccacagcagctgatgaggtgacagctcatctggcagctgcaggtcctgtgggaatggccgctgctgctgctgtggcaacaggaaagaaacggaaacggcctcatgtatttgagtctaatccatctatccggaagaggcaacaaacacgtttgcttcggaaacttcgagccacgttagatgaatatactactcgtgtgggacagcaagctattgtcctctgtatctcaccctccaaacctaaccctgtctttaaagtgtttggtgcagcacctttggagaatgtggtgcgtaagtacaagagcatgatcctggaagacctggagtctgctctggcagaacacgcccctgcgccacaggaggttaactcagaactgccgcctctcaccatcgacggaattccagtctctgtggacaaaatgacccaggcccagcttcgggcatttatcccagagatgctcaagtactctacaggtcggggaaaaccaggctgggggaaagaaagctgcaagcccatctggtggcctgaagatatcccctgggcaaatgtccggagtgatgtccgcacagaagagcaaaagcagagggtttcatggacccaggcactacggaccatagttaaaaactgttataaacagcatgggcgggaagaccttttgtatgcctttgaagatcagcaaacgcaaacacaggccacagccacacatagtatagctcatcttgtaccatcacagactgtagtccagacttttagtaaccctgatggcactgtctcacttatccaggttggtacgggggcaacagtagccacattggctgatgcttcagaattgccaaccacggtcaccgttgcccaagtgaattattctgccgtggctgatggagaggtggaacaaaattgggccacgttacagggaggtgagatgaccatccagacgacgcaagcatcagaggccacccaggcggtggcatcgttggcagaggccgcagtggcagcttctcaggagatgcagcagggagctacagtcactatggcgcttaacagcgaagctgccgcccatgctgtcgccaccctggctgaggccaccttacaaggtgggggacagatcgtcttgtctggggaaaccgcagcagccgtcggagcacttactggagtccaagatgctaatggcctctttatggcagatcgtgcaggtcgcaagtggatcctgactgacaaagccacaggcctggtccagatccctgtgagcatgtaccagactgtggtgaccagcctcgcccagggcaacggaccagtgcaggtggccatggcccctgtgaccaccaggatatcagacagcgcagtcaccatggacggccaagctgtggaggtggtgacattggaacagtgacatacagccatattatggcatcgttttctagtctacttcaaaattttttacacgtttgcagaggtgcaatcaaatggaattaagtctctcgactttggaaggaaagttttgttaacctttttttttttaaaaggaagaaagcggattttggaattgcattttttaaagcaccactcttgattttctgggattggtgaagaaactgcattgtcaatttcactgtcccaaaaaagccaaattgtggcaggacttctttctgcggaaatgtgtgtgtatacttatgtgtgtgtatgtgtgagtgtgaatatatgtatatgtgtacatatggacatacacatttacatatatataaagtatatatatacatatatatatatatatgtatgaaacccgcatggaattatctgtatgaaatcaaggtgcgctgtggaaacaataattcacccagtttagtgggtggtagggtacgtggccagacacagtcacccagtttttgttcataccagggtcatgcgttgagctactgacaaactcaggcggaggtgaccatgcccttcaccaaagctgcctcccagtggccacacagaactctccctgctggactcacctgaggaaagaggctccagcatggggtgggtcagagatgtgcttgcaaggtccagggactgcgtggtctgccagctgagatgctcctcgggctggcccaggtgctgaccttgccacaggcagatgaatgtcttgaaagctcccgggcctcagcctcccatctcctctccttcccaggaatccttgatctcatgactattaaaatgttgctctggttttaaggtcaaaaaaaaaaaaaaaaaaaaaaaaaaaaaaaaaaaa

SEQ ID NO: 73 - protein, 522 aa, homo sapiens
MEEHGVTQTEHMATIEAHAVAQQVQQVHVATYTEHSMLSADEDSPSSPEDTSYDDSDILNSTAADEVTAHLAAAGPVGMAAAAAVATGKKRKRPHVFESNPSIRKRQQTRLLRKLRATLDEYTTRVGQQAIVLCISPSKPNPVFKVFGAAPLENVVRKYKSMILEDLESALAEHAPAPQEVNSELPPLTIDGIPVSVDKMTQAQLRAFIPEMLKYSTGRGKPGWGKESCKPIWWPEDIPWANVRSDVRTEEQKQRVSWTQALRTIVKNCYKQHGREDLLYAFEDQQTQTQATATHSIAHLVPSQTVVQTFSNPDGTVSLIQVGTGATVATLADASELPTTVTVAQVNYSAVADGEVEQNWATLQGGEMTIQTTQASEATQAVASLAEAAVAASQEMQQGATVTMALNSEAAAHAVATLAEATLQGGGQIVLSGETAAAVGALTGVQDANGLFMADRAGRKWILTDKATGLVQIPVSMYQTVVTSLAQGNGPVQVAMAPVTTRISDSAVTMDGQAVEVVTLEQ

SEQ ID NO: 74 - DNA, 1806 bp, homo sapiens
ggccggagggagcccgcgctcggggcggcggctggaggcagcgcaccgagttcccgcgaggatccatgacctgacggggccccggagccgcgctgcctctcgggtgtcctgggtcggtggggagcccagtgctcgcaggccggcgggcgggccggagggctgcagtctccctcgcggtgagaggaaggcggaggagcgggaaccgcggcggcgctcgcgcggcgcctgcggggggaagggcagttccgggccgggccgcgcctcagcagggcggcggctcccagcgcagtctcagggcccgggtggcggcggcgactggagaaatcaagttgtgcggtcggtgatgcccgagtgagcggggggcctgggcctctgcccttaggaggcaactcccacgcaggccgcaaagggctctcgcggccgagaggcttcgtttcggtttcgcggcggcggcggcgttgttggctgaggggacccgggacacctgaatgcccccggccccggctcctccgacgcgatggggaaggtgctatccaaaatcttcgggaacaaggaaatgcggatcctcatgttgggcctggacgcggccggcaagacaacaatcctgtacaagttgaagctgggccagtcggtgaccaccattcccactgtgggtttcaacgtggagacggtgacttacaaaaatgtcaagttcaacgtatgggatgtgggcggccaggacaagatccggccgctctggcggcattactacactgggacccaaggtctcatcttcgtagtggactgcgccgaccgcgaccgcatcgatgaggctcgccaggagctgcaccgcattatcaatgaccgggagatgagggacgccataatcctcatcttcgccaacaagcaggacctgcccgatgccatgaaaccccacgagatccaggagaaactgggcctgacccggattcgggacaggaactggtatgtgcagccctcctgtgccacctcaggggacggactctatgaggggctcacatggttaacctctaactacaaatcttaatgagcattctccacccatcccctggaaggagagaaatcaaaaacccattcataggattatcgccaccatcacctctttcaattgccactttctcttcttttgaatttgaactctggagttactgttctacagtttggcggggacggggcttgggggttttctcttttgtttgtttccctttctttttcctttttttttttttttttttgttggctttgcgttaggatggctctgatctgacatttgacatgaacacaaagttgccaagatgctccttgttgacttccagcagaatgggaatgggggaaacacagcagttcttgggtaaaagtccctttgtaataataggtttgggatttttttatttcgagagaatctttcattttcctatgtatgcttttttccttttttgcccagtttccttatcacttgctgtagatggcttattttgcattcatgcagactatgttgcaagtctgtttcatctagtaaactgaaaattattgcttaatcaaactgccgtttgtcttttatatttaaggccttccccccccttccttatgagttctaacttagtaatttcaaatgtgaccttttatatctaagaccagtatagtaaacttagcccacagtggcaaataatgagtaatattgtaatatgttccagttgcacctcagtatgttaaacaggtaatgtaagaagttctctgaaatgtcagcaagtaagttctgaaacacatcatgcatgagtaggaataaac

SEQ ID NO: 75 - protein, 175 aa, homo sapiens
MGKVLSKIFGNKEMRILMLGLDAAGKTTILYKLKLGQSVTTIPTVGFNVETVTYKNVKFNVWDVGGQDKIRPLWRHYYTGTQGLIFVVDCADRDRIDEARQELHRIINDREMRDAIILIFANKQDLPDAMKPHEIQEKLGLTRIRDRNWYVQPSCATSGDGLYEGLTWLTSNYKS

SEQ ID NO: 76 - DNA, 1407 bp, homo sapiens
ctctccccggcccgatccgcccgccggctccccctcccccgatccctcgggtcccgggatgggggggcggtgaggcaggcacagccccccgcccccatggccgcccgtcggagccagaggcggagggggcgccggggggagccgggcaccgccctgctggtcccgctcgcgctgggcctgggcctggcgctggcctgcctcggcctcctgctggccgtggtcagtttggggagccgggcatcgctgtccgcccaggagcctgcccaggaggagctggtggcagaggaggaccaggacccgtcggaactgaatccccagacagaagaaagccaggatcctgcgcctttcctgaaccgactagttcggcctcgcagaagtgcacctaaaggccggaaaacacgggctcgaagagcgatcgcagcccattatgaagttcatccacgacctggacaggacggagcgcaggcaggtgtggacgggacagtgagtggctgggaggaagccagaatcaacagctccagccctctgcgctacaaccgccagatcggggagtttatagtcacccgggctgggctctactacctgtactgtcaggtgcactttgatgaggggaaggctgtctacctgaagctggacttgctggtggatggtgtgctggccctgcgctgcctggaggaattctcagccactgcggcgagttccctcgggccccagctccgcctctgccaggtgtctgggctgttggccctgcggccagggtcctccctgcggatccgcaccctcccctgggcccatctcaaggctgcccccttcctcacctacttcggactcttccaggttcactgaggggccctggtctccccgcagtcgtcccaggctgccggctcccctcgacagctctctgggcacccggtcccctctgccccaccctcagccgctctttgctccagacctgcccctccctctagaggctgcctgggcctgttcacgtgttttccatcccacataaatacagtattcccactcttatcttacaactcccccaccgcccactctccacctcactagctccccaatccctgaccctttgaggcccccagtgatctcgactcccccctggccacagacccccagggcattgtgttcactgtactctgtgggcaaggatgggtccagaagaccccacttcaggcactaagaggggctggacctggcggcaggaagccaaagagactgggcctaggccaggagttcccaaatgtgaggggcgagaaacaagacaagctcctcccttgagaattccctgtggatttttaaaacagatattatttttattattattgtgacaaaatgttgataaatggatattaaatagaataagtcataaaaaaaaaaaaaaaaaaaaaa

SEQ ID NO: 77 - protein, 249 aa, homo sapiens
MAARRSQRRRGRRGEPGTALLVPLALGLGLALACLGLLLAVVSLGSRASLSAQEPAQEELVAEEDQDPSELNPQTEESQDPAPFLNRLVRPRRSAPKGRKTRARRAIAAHYEVHPRPGQDGAQAGVDGTVSGWEEARINSSSPLRYNRQIGEFIVTRAGLYYLYCQVHFDEGKAVYLKLDLLVDGVLALRCLEEFSATAASSLGPQLRLCQVSGLLALRPGSSLRIRTLPWAHLKAAPFLTYFGLFQVH

SEQ ID NO: 78 - DNA, 2444 bp, homo sapiens
ggcacgaggcggggccgggtcgcagctgggcccgcggcatggacgaactgttccccctcatcttcccggcagagcagcccaagcagcggggcatgcgcttccgctacaagtgcgaggggcgctccgcgggcagcatcccaggcgagaggagcacagataccaccaagacccaccccaccatcaagatcaatggctacacaggaccagggacagtgcgcatctccctggtcaccaaggaccctcctcaccggcctcacccccacgagcttgtaggaaaggactgccgggatggcttctatgaggctgagctctgcccggaccgctgcatccacagtttccagaacctgggaatccagtgtgtgaagaagcgggacctggagcaggctatcagtcagcgcatccagaccaacaacaaccccttccaagttcctatagaagagcagcgtggggactacgacctgaatgctgtgcggctctgcttccaggtgacagtgcgggacccatcaggcaggcccctccgcctgccgcctgtcctttctcatcccatctttgacaatcgtgcccccaacactgccgagctcaagatctgccgagtgaaccgaaactctggcagctgcctcggtggggatgagatcttcctactgtgtgacaaggtgcagaaagaggacattgaggtgtatttcacgggaccaggctgggaggcccgaggctccttttcgcaagctgatgtgcaccgacaagtggccattgtgttccggacccctccctacgcagaccccagcctgcaggctcctgtgcgtgtctccatgcagctgcggcggccttccgaccgggagctcagtgagcccatggaattccagtacctgccagatacagacgatcgtcaccggattgaggagaaacgtaaaaggacatatgagaccttcaagagcatcatgaagaagagtcctttcagcggacccaccgacccccggcctccacctcgacgcattgctgtgccttcccgcagctcagcttctgtccccaagccagcaccccagccctatccctttacgtcatccctgagcaccatcaactatgatgagtttcccaccatggtgtttccttctgggcagatcagccaggcctcggccttggccccggcccctccccaagtcctgccccaggctccagcccctgcccctgctccagccatggtatcagctctggcccaggccccagcccctgtcccagtcctagccccaggccctcctcaggctgtggccccacctgcccccaagcccacccaggctggggaaggaacgctgtcagaggccctgctgcagctgcagtttgatgatgaagacctgggggccttgcttggcaacagcacagacccagctgtgttcacagacctggcatccgtcgacaactccgagtttcagcagctgctgaaccagggcatacctgtggccccccacacaactgagcccatgctgatggagtaccctgaggctataactcgcctagtgacagcccagaggccccccgacccagctcctgctccactgggggccccggggctccccaatggcctcctttcaggagatgaagacttctcctccattgcggacatggacttctcagccctgctgagtcagatcagctcctaagggggtgacgcctgccctccccagagcactggttgcaggggattgaagccctccaaaagcacttacggattctggtggggtgtgttccaactgcccccaactttgtggatgtcttccttggaggggggagccatattttattcttttattgtcagtatctgtatctctctctctttttggaggtgcttaagcagaagcattaacttctctggaaaggggggagctggggaaactcaaacttttcccctgtcctgatggtcagctcccttctctgtagggaactgtggggtcccccatccccatcctccagcttctggtactctcctagagacagaagcaggctggaggtaaggcctttgagcccacaaagccttatcaagtgtcttccatcatggattcattacagcttaatcaaaataacgccccagataccagcccctgtatggcactggcattgtccctgtgcctaacaccagcgtttgaggggctgccttcctgccctacagaggtctctgccggctctttccttgctcaaccatggctgaaggaaacagtgcaacagcactggctctctccaggatccagaaggggtttggtctggacttccttgctctcccctcttctcaagtgccttaatagtagggtaagttgttaagagtgggggagagcaggctggcagctctccagtcaggaggcatagtttttagtgaacaatcaaagcacttggactcttgctctttctactctgaactaataaagctgttgccaagctggacggcacgagctcgtgcc

SEQ ID NO: 79 - protein, 537 aa, homo sapiens
MDELFPLIFPAEQPKQRGMRFRYKCEGRSAGSIPGERSTDTTKTHPTIKINGYTGPGTVRISLVTKDPPHRPHPHELVGKDCRDGFYEAELCPDRCIHSFQNLGIQCVKKRDLEQAISQRIQTNNNPFQVPIEEQRGDYDLNAVRLCFQVTVRDPSGRPLRLPPVLSHPIFDNRAPNTAELKICRVNRNSGSCLGGDEIFLLCDKVQKEDIEVYFTGPGWEARGSFSQADVHRQVAIVFRTPPYADPSLQAPVRVSMQLRRPSDRELSEPMEFQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTAQRPPDPAPAPLGAPGLPNGLLSGDEDFSSIADMDFSALLSQISS

SEQ ID NO: 80 - DNA, 2236 bp, homo sapiens
gctgttgcaacactgcctcactcttcccctcccaccttctctcccctcctctctgctttaattttctcagaattctctggactgaggctccagttctggcctttggggttcaagatcactgggaccaggccgtgatctctatgcccgagtctcaaccctcaactgtcaccccaaggcacttgggacgtcctggacagaccgagtcccgggaagccccagcactgccgctgccacactgccctgagcccaaatgggggagtgagaggccatagctgtctggcatgggcctctccaccgtgcctgacctgctgctgccactggtgctcctggagctgttggtgggaatatacccctcaggggttattggactggtccctcacctaggggacagggagaagagagatagtgtgtgtccccaaggaaaatatatccaccctcaaaataattcgatttgctgtaccaagtgccacaaaggaacctacttgtacaatgactgtccaggcccggggcaggatacggactgcagggagtgtgagagcggctccttcaccgcttcagaaaaccacctcagacactgcctcagctgctccaaatgccgaaaggaaatgggtcaggtggagatctcttcttgcacagtggaccgggacaccgtgtgtggctgcaggaagaaccagtaccggcattattggagtgaaaaccttttccagtgcttcaattgcagcctctgcctcaatgggaccgtgcacctctcctgccaggagaaacagaacaccgtgtgcacctgccatgcaggtttctttctaagagaaaacgagtgtgtctcctgtagtaactgtaagaaaagcctggagtgcacgaagttgtgcctaccccagattgagaatgttaagggcactgaggactcaggcaccacagtgctgttgcccctggtcattttctttggtctttgccttttatccctcctcttcattggtttaatgtatcgctaccaacggtggaagtccaagctctactccattgtttgtgggaaatcgacacctgaaaaagagggggagcttgaaggaactactactaagcccctggccccaaacccaagcttcagtcccactccaggcttcacccccaccctgggcttcagtcccgtgcccagttccaccttcacctccagctccacctatacccccggtgactgtcccaactttgcggctccccgcagagaggtggcaccaccctatcagggggctgaccccatccttgcgacagccctcgcctccgaccccatccccaacccccttcagaagtgggaggacagcgcccacaagccacagagcctagacactgatgaccccgcgacgctgtacgccgtggtggagaacgtgcccccgttgcgctggaaggaattcgtgcggcgcctagggctgagcgaccacgagatcgatcggctggagctgcagaacgggcgctgcctgcgcgaggcgcaatacagcatgctggcgacctggaggcggcgcacgccgcggcgcgaggccacgctggagctgctgggacgcgtgctccgcgacatggacctgctgggctgcctggaggacatcgaggaggcgctttgcggccccgccgccctcccgcccgcgcccagtcttctcagatgaggctgcgcccctgcgggcagctctaaggaccgtcctgcgagatcgccttccaaccccacttttttctggaaaggaggggtcctgcaggggcaagcaggagctagcagccgcctacttggtgctaacccctcgatgtacatagcttttctcagctgcctgcgcgccgccgacagtcagcgctgtgcgcgcggagagaggtgcgccgtgggctcaagagcctgagtgggtggtttgcgaggatgagggacgctatgcctcatgcccgttttgggtgtcctcaccagcaaggctgctcgggggcccctggttcgtccctgagcctttttcacagtgcataagcagttttttttgtttttgttttgttttgttttgtttttaaatcaatcatgttacactaatagaaacttggcactcctgtgccctctgcctggacaagcacatagcaagctgaactgtcctaaggcaggggcgagcacggaacaatggggccttcagctggagctgtggacttttgtacatacactaaaattctgaagttaaagctctgctcttggaaaaaaaaaaaaaaaaaaaaaaaaaaaaaaaaa

SEQ ID NO: 81 - protein, 455 aa, homo sapiens
MGLSTVPDLLLPLVLLELLVGIYPSGVIGLVPHLGDREKRDSVCPQGKYIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSLCLNGTVHLSCQEKQNTVCTCHAGFFLRENECVSCSNCKKSLECTKLCLPQIENVKGTEDSGTTVLLPLVIFFGLCLLSLLFIGLMYRYQRWKSKLYSIVCGKSTPEKEGELEGTTTKPLAPNPSFSPTPGFTPTLGFSPVPSSTFTSSSTYTPGDCPNFAAPRREVAPPYQGADPILATALASDPIPNPLQKWEDSAHKPQSLDTDDPATLYAVVENVPPLRWKEFVRRLGLSDHEIDRLELQNGRCLREAQYSMLATWRRRTPRREATLELLGRVLRDMDLLGCLEDIEEALCGPAALPPAPSLLR

SEQ ID NO: 82 - DNA, 1735 bp, homo sapiens
agagactcgtgccgaatggcacgaggccgacgggcccgcggggccggcgccgccatggcggccgtgtttgatttggatttggagacggaggaaggcagcgagggcgagggcgagccagagctcagccccgcggacgcatgtccccttgccgagttgagggcagctggcctagagcctgtgggacactatgaagaggtggagctgactgagaccagcgtgaacgttggcccagagcgcatcgggccccactgctttgagctgctgcgtgtgctgggcaaggggggctatggcaaggtgttccaggtgcgaaaggtgcaaggcaccaacttgggcaaaatatatgccatgaaagtcctaaggaaggccaaaattgtgcgcaatgccaaggacacagcacacacacgggctgagcggaacattctagagtcagtgaagcacccctttattgtggaactggcctatgccttccagactggtggcaaactctacctcatccttgagtgcctcagtggtggcgagctcttcacgcatctggagcgagagggcatcttcctggaagatacggcctgcttctacctggctgagatcacgctggccctgggccatctccactcccagggcatcatctaccgggacctcaagcccgagaacatcatgctcagcagccagggccacatcaaactgaccgactttggactctgcaaggagtctatccatgagggcgccgtcactcacaccttctgcggcaccattgagtacatggcccctgagattctggtgcgcagtggccacaaccgggctgtggactggtggagcctgggggccctgatgtacgacatgctcactggatcgccgccctttaccgcagagaaccggaagaaaaccatggataagatcatcaggggcaagctggcactgcccccctacctcaccccagatgcccgggaccttgtcaaaaagtttctgaaacggaatcccagccagcggattgggggtggcccaggggatgctgctgatgtgcagagacatccctttttccggcacatgaattgggacgaccttctggcctggcgtgtggacccccctttcaggccctgtctgcagtcagaggaggacgtgagccagtttgatacccgcttcacacggcagacgccggtggacagtcctgatgacacagccctcagcgagagtgccaaccaggccttcctgggcttcacatacgtggcgccgtctgtcctggacagcatcaaggagggcttctccttccagcccaagctgcgctcacccaggcgcctcaacagtagcccccgggtccccgtcagccccctcaagttctccccttttgaggggtttcggcccagccccagcctgccggagcccacggagctacctctacctccactcctgccaccgccgccgccctcgaccaccgcccctctccccatccgtcccccctcagggaccaagaagtccaagaggggccgtgggcgtccagggcgctaggaagccgggtgggggtgagggtagcccttgagccctgtccctgcggctgtgagagcagcaggaccctgggccagttccagagacctgggggtgtgtctgggggtggggtgtgagtgcgtatgaaagtgtgtgtctgctggggcagctgtgcccctgaatcatgggcacggagggccgcccgccacaccccgcgctcaactgctcccgtggaagattaaagggctgaatcatg

SEQ ID NO: 83 - protein, 495 aa, homo sapiens
MARGRRARGAGAAMAAVFDLDLETEEGSEGEGEPELSPADACPLAELRAAGLEPVGHYEEVELTETSVNVGPERIGPHCFELLRVLGKGGYGKVFQVRKVQGTNLGKIYAMKVLRKAKIVRNAKDTAHTRAERNILESVKHPFIVELAYAFQTGGKLYLILECLSGGELFTHLEREGIFLEDTACFYLAEITLALGHLHSQGIIYRDLKPENIMLSSQGHIKLTDFGLCKESIHEGAVTHTFCGTIEYMAPEILVRSGHNRAVDWWSLGALMYDMLTGSPPFTAENRKKTMDKIIRGKLALPPYLTPDARDLVKKFLKRNPSQRIGGGPGDAADVQRHPFFRHMNWDDLLAWRVDPPFRPCLQSEEDVSQFDTRFTRQTPVDSPDDTALSESANQAFLGFTYVAPSVLDSIKEGFSFQPKLRSPRRLNSSPRVPVSPLKFSPFEGFRPSPSLPEPTELPLPPLLPPPPPSTTAPLPIRPPSGTKKSKRGRGRPGR

SEQ ID NO: 84 - DNA, 2169 bp, homo sapiens
gccagagcggcgcggcctggaagaggccagggcccgggggaggcgacggcagcggcggcggctggggcagcccgggcagcccgagccccgcagcctgggcctgtgctcggcgccatgagcggcggcgggccttcgggaggcggccctgggggctcgggcagggcgcggactagctcgttcgcggagcccggcggcggaggcggaggaggcggcggcggccccggaggctcggcctccggcccaggcggcaccggcggcggaaaggcatctgtcggggccatgggtgggggcgtcggggcctcgagctccgggggtggacccggcggcagcggcggaggaggcagcggaggccccggcgcaggcactagcttcccgccgcccggggtgaagctgggccgtgacagcgggaaggtgaccacagtcgtagccactctaggccaaggcccagagcgctcccaagaagtggcttacacggacatcaaagtgattggcaatggctcatttggggtcgtgtaccaggcacggctggcagagaccagggaactagtcgccatcaagaaggttctccaggacaagaggttcaagaaccgagagctgcagatcatgcgtaagctggaccactgcaatattgtgaggctgagatactttttctactccagtggcgagaagaaagacgagctttacctaaatctggtgctggaatatgtgcccgagacagtgtaccgggtggcccgccacttcaccaaggccaagttgaccatccctatcctctatgtcaaggtgtacatgtaccagctcttccgcagcttggcctacatccactcccagggcgtgtgtcaccgcgacatcaagccccagaacctgctggtggaccctgacactgctgtcctcaagctctgcgattttggcagtgcaaagcagttggtccgaggggagcccaatgtctcctacatctgttctcgctactaccgggccccagagctcatctttggagccactgattacacctcatccatcgatgtttggtcagctggctgtgtactggcagagctcctcttgggccagcccatcttccctggggacagtggggtggaccagctggtggagatcatcaaggtgctgggaacaccaacccgggaacaaatccgagagatgaaccccaactacacggagttcaagttccctcagattaaagctcacccctggacaaaggtgttcaaatctcgaacgccgccagaggccatcgcgctctgctctagcctgctggagtacaccccatcctcaaggctctccccactagaggcctgtgcgcacagcttctttgatgaactgcgatgtctgggaacccagctgcctaacaaccgcccacttccccctctcttcaacttcagtgctggtgaactctccatccaaccgtctctcaacgccattctcatccctcctcacttgaggtcccccagcggcactaccaccctcaccccgtcctcacaagctttaactgagactccgaccagctcagactggcagtcgaccgatgccacacctaccctcactaactcctcctgagggccccaccaagcacccttccacttccatctgggagccccaagagggcgtgggaaggggggccatagcccatcaagctcctgccctggctgggcccctagactagagggcagaggtaaatgagtccctgtccccacctccagtccctccctcaccagcctcacccctgtggtgggctttttaagaggattttaactggttgtggggagggaagagaaggacagggtgttggggggatgaggacctcctacccccttggccccctcccctcccccagacctccacctcctccagaccccctcccctcctgtgtcccttgtaaatagaaccagcccagcccgtctcctcttcccttccctggcccccgggtgtaaatagattgttataatttttttcttaaagaaaacgtcgattcgcaccgtccaacctgccccgcccctcctacagctgtaactcccctcctgtcctctgcccccaaggtctactccctcctcaccccaccctggagggccaggggagtggagagagctcctgatgtcttagtttccacagtaaggtttgcctgtgtacagacctccgttcaataaattattggcatgaaaa

SEQ ID NO: 85 - protein, 483 aa, homo sapiens
MSGGGPSGGGPGGSGRARTSSFAEPGGGGGGGGGGPGGSASGPGGTGGGKASVGAMGGGVGASSSGGGPGGSGGGGSGGPGAGTSFPPPGVKLGRDSGKVTTVVATLGQGPERSQEVAYTDIKVIGNGSFGVVYQARLAETRELVAIKKVLQDKRFKNRELQIMRKLDHCNIVRLRYFFYSSGEKKDELYLNLVLEYVPETVYRVARHFTKAKLTIPILYVKVYMYQLFRSLAYIHSQGVCHRDIKPQNLLVDPDTAVLKLCDFGSAKQLVRGEPNVSYICSRYYRAPELIFGATDYTSSIDVWSAGCVLAELLLGQPIFPGDSGVDQLVEIIKVLGTPTREQIREMNPNYTEFKFPQIKAHPWTKVFKSRTPPEAIALCSSLLEYTPSSRLSPLEACAHSFFDELRCLGTQLPNNRPLPPLFNFSAGELSIQPSLNAILIPPHLRSPSGTTTLTPSSQALTETPTSSDWQSTDATPTLTNSS

SEQ ID NO: 86 - DNA, 1689 bp, homo sapiens
gcctccgggagaggagccgcacccggccggcccggccccagccccatggacctccgagcaggggactcgtgggggatgttagcgtgcctgtgcacggtgctctggcacctccctgcagtgccagctctcaatcgcacaggggacccagggcctggcccctccatccagaaaacctatgacctcacccgctacctggagcaccaactccgcagcttggctgggacctatctgaactacctgggcccccctttcaacgagccagacttcaaccctccccgcctgggggcagagactctgcccagggccactgttgacttggaggtgtggcgaagcctcaatgacaaactgcggctgacccagaactacgaggcctacagccaccttctgtgttacttgcgtggcctcaaccgtcaggctgccactgctgagctgcgccgcagcctggcccacttctgcaccagcctccagggcctgctgggcagcattgcgggcgtcatggcagctctgggctacccactgccccagccgctgcctgggactgaacccacttggactcctggccctgcccacagtgacttcctccagaagatggacgacttctggctgctgaaggagctgcagacctggctgtggcgctcggccaaggacttcaaccggctcaagaagaagatgcagcctccagcagctgcagtcaccctgcacctgggggctcatggcttctgacttctgaccttctcctcttcgctcccccttcaaaccctgctcccactttgtgagagccagccctgtatgccaacacctgttgagccaggagacagaagctgtgagcctctggccctttcctggaccggctgggcgtgtgatgcgatcagccctgtctcctccccacctcccaaaggtctaccgagctggggaggaggtacagtaggccctgtcctgtcctgtttctacaggaagtcatgctcgagggagtgtgaagtggttcaggttggtgcagaggcgctcatggcctcctgcttcttgcctaccacttggccagtgcccacccagcccctcaggtggcacatctggagggcaggggttgaggggccaccaccacacatgcctttctggggtgaagccctttggctgccccactctccttggatgggtgttgctcccttatccccaaatcactctatacatccaattcaggaaacaaacatggtggcaattctacacaaaaagagatgagattaacagtgcagggttggggtctgcattggaggtgccctataaaccagaagagaaaatactgaaagcacaggggcagggacagaccagaccagacccaggagtctccaaagcacagagtggcaaacaaaacccgagctgagcatcaggaccttgcctcgaattgtcttccagtattacggtgcctcttctctgccccctttcccagggtatctgtgggttgccaggctggggagggcaaccatagccacaccacaggatttcctgaaagtttacaatgcagtagcattttggggtgtagggtggcagctccccaaggccctgccccccagccccacccactcatgactctaagtgtgttgtattaatatttatttatttggagatgttatttattagatgatatttattgcagaatttctattcttgtattaacaaataaaatgcttgccccag

SEQ ID NO: 87 - protein, 225 aa, homo sapiens
MDLRAGDSWGMLACLCTVLWHLPAVPALNRTGDPGPGPSIQKTYDLTRYLEHQLRSLAGTYLNYLGPPFNEPDFNPPRLGAETLPRATVDLEVWRSLNDKLRLTQNYEAYSHLLCYLRGLNRQAATAELRRSLAHFCTSLQGLLGSIAGVMAALGYPLPQPLPGTEPTWTPGPAHSDFLQKMDDFWLLKELQTWLWRSAKDFNRLKKKMQPPAAAVTLHLGAHGF

SEQ ID NO: 88 - DNA, 3073 bp, homo sapiens
ggcctctggccgcctggctccaacatcaagcaccgggctccgagtggccgggatcagcgccccgaggcagaggccggagggcgcgcgcactgctaggaagtgctggtcccccgcgccgctctgccagcttggtcccccggcagacgcccctgtacgatcgccgctcgccccgcgggcgaggctgcggtggacagcgcggggctccggctggctcgccttcccgcctgccgtgtcctgctgagcgaccctggagtacacatccagatgccagcccagctaccacaggggatccctctgggagactgaaagtacaggttctggggcccaggttgaagccgaccaaccctgagcctcaggccaggggaatggcagcccccttggaggcccaggaccaggcccctggggagggagaagggcttctgattgtgaaagtggaagattcctcctgggaacaggaatctgcccagcatgaggatggcagggattccgaagcctgccgccagcgcttccggcaattctgctacggggatgtgcatgggcctcatgaggccttcagccagctctgggagctctgctgccgctggctgcggcccgagctgcgtaccaaggagcagatcctggagctgctggtgctggagcagttcctgacagtgctgccaggggagatccagggctgggtgcgtgagcagcacccgggaagcggtgaggaggctgtcgccttggtggaggacctacagaagcagccagtgaaagcctggcgacaggatgtgccctcggaggaggcggaacccgaggctgcaggccggggatcccaggccacggggcctcccccgacggtgggggcacggaggcggccgtctgttccccaggagcagcacagccatagcgcccagcctcctgctcttcttaaagagggtcgtcccggagagacgacggacacctgctttgtctctggggtccatggacctgtggcattgggagacatcccattctatttctcccgggaagaatggggcaccctggaccctgctcagcgggatctcttctgggacataaagcgggagaactcccggaacaccaccctgggttttgggctcaaaggccaaagtgagaagtccctgctgcaggagatggtgccggtggtgccaggccagacaggcagcgacgtgactgtgtcctggagccccgaggaggctgaggcctgggagagcgagaaccggccgagggcggccctgggcccagtggtgggcgcgcgacgggggcggccacccactcgccggcgccagttccgggacctggcagccgagaagccgcacagctgcgggcagtgtggaaagcgcttccgctggggctcggacctggcgcggcaccagcgcacgcacacgggcgagaagccacacaagtgccctgagtgcgacaagagcttccgcagctcctcggacctggtgcgccaccaaggcgtgcacacgggcgagaagcccttctcctgttccgagtgcggcaagagcttcagccgcagcgcctacctggccgaccaccagcgcatacacacgggcgagaagcctttcggctgcagcgactgcggcaagagcttctcgctgcgctcctacctgctggaccatcggcgtgtgcacaccggtgagcggcccttcggctgcggagagtgcgacaagagcttcaagcagcgcgcgcacctcatcgcgcatcagagcctgcacgccaagatggcccagcccgtggggtgagcagctggcttggccggaaacccgggggaggcccagccacggcacatcctgctttgttcaccactgggactctccttccatctgtggccacctcccgggctgtccgagggaccccagggtacctcacactcggagctcgcctgccctgcttggctctgaggacctgcccagcgctcaaagggaacggaagccttcccctcccgcccccgatcttgtcctctttcccccttctgcgcctagcgttcctcttcccctctagtttcctggagccccaacacattcctggcagggacagcagggtggcaaggactcaggtctaggtcccttcccagaagcccccgagcctcatttgactgtgtggctctttggcccccaccctgtggggtgggtccatgggtcaggcctctgccctaccaacctgtgcctttcagtgggcgtggaggactggccttggccccccagggggctgctggactttgggagagacagcccacacctgtgggaccgcgggtcttagtcacggcggcaggggctttctggccccctcccactcccgtttccaggccatgaccactctgccctgtcctggccatacggactcggcctgcctttgccctcggcctacttgccctagcatgaggctctgagagccacctgcccaccaatctggtgaggataatggtggctccagcgacaggaggccaaccctggagaccaagaacagggcgcctggctgccatcttttcctccagaggtggggctgcaccagactcagcactagcactccatcagcactagcacctcactccatcagcactagcacctcactccatcggccccggcaccctgctccatcggcactggcgccctgctccatcggcactaatgctccactcggcgccccactccatcggccccgctccatcggcactaatgccccactcggcgccccactccatcagcactaatgctccactccattggcactaacgccccaactccagcggcactaatgacccgctcctttgacattggtgccccactccatcagcactaacgccctgctccatcggcactggtgtcccactccattgtcactaacgtccggctccatcggcactaccaccccgctccatcatcactatgtccagctccgtcggcactaccaccctgctccatcatcactacgtccagctccaacggcactggtgccccattccatcggcactaacgccccgctccaccggcaccagtgcctcgctccattggcaccaacgcccagctccaccggtactggctccctgctccatcggcactaacgccctgct

SEQ ID NO: 89 - protein, 459 aa, homo sapiens
MAAPLEAQDQAPGEGEGLLIVKVEDSSWEQESAQHEDGRDSEACRQRFRQFCYGDVHGPHEAFSQLWELCCRWLRPELRTKEQILELLVLEQFLTVLPGEIQGWVREQHPGSGEEAVALVEDLQKQPVKAWRQDVPSEEAEPEAAGRGSQATGPPPTVGARRRPSVPQEQHSHSAQPPALLKEGRPGETTDTCFVSGVHGPVALGDIPFYFSREEWGTLDPAQRDLFWDIKRENSRNTTLGFGLKGQSEKSLLQEMVPVVPGQTGSDVTVSWSPEEAEAWESENRPRAALGPVVGARRGRPPTRRRQFRDLAAEKPHSCGQCGKRFRWGSDLARHQRTHTGEKPHKCPECDKSFRSSSDLVRHQGVHTGEKPFSCSECGKSFSRSAYLADHQRIHTGEKPFGCSDCGKSFSLRSYLLDHRRVHTGERPFGCGECDKSFKQRAHLIAHQSLHAKMAQPVG

SEQ ID NO: 90 - DNA, 1501 bp, homo sapiens
ggagagagccgccgagccgagccgagccccagctccagcaagagcgcgggcgggtggcccaggcacgcagcggtgaggaccgcggccacagctcggcgccaaccaccgcgggcctcccagccagccccgcggcggggcagccgcaggagccctggctgtggtcggggggcagtgggccatgctgggggcagtggaaggccccaggtggaagcaggcggaggacattagagacatctacgacttccgagatgttctgggcacgggggccttctcggaggtgatcctggcagaagataagaggacgcagaagctggtggccatcaaatgcattgccaaggaggccctggagggcaaggaaggcagcatggagaatgagattgctgtcctgcacaagatcaagcaccccaacattgtagccctggatgacatctatgagagtgggggccacctctacctcatcatgcagctggtgtcgggtggggagctctttgaccgtattgtggaaaaaggcttctacacggagcgggacgccagccgcctcatcttccaggtgctggatgctgtgaaatacctgcatgacctgggcattgtacaccgggatctcaagccagagaatctgctgtactacagcctggatgaagactccaaaatcatgatctccgactttggcctctccaagatggaggacccgggcagtgtgctctccaccgcctgtggaactccgggatacgtggcccctgaagtcctggcccagaagccctacagcaaggctgtggattgctggtccataggtgtcatcgcctacatcttgctctgcggttaccctcccttctatgacgagaatgatgccaaactctttgaacagattttgaaggccgagtacgagtttgactctccttactgggacgacatctctgactctgccaaagatttcatccggcacttgatggagaaggacccagagaaaagattcacctgtgagcaggccttgcagcacccatggattgcaggagatacagctctagataagaatatccaccagtcggtgagtgagcagatcaagaagaactttgccaagagcaagtggaagcaagccttcaatgccacggctgtggtgcggcacatgaggaaactgcagctgggcaccagccaggaggggcaggggcagacggcgagccatggggagctgctgacaccagtggctggggggccggcagctggctgttgctgtcgagactgctgcgtggagccgggcacagaactgtcccccacactgccccaccagctctagggccctggacctcgggtcatgatcctctgcgtgggagggcttgggggcagcctgctccccttccctccctgaaccgggagtttctctgccctgtcccctcctcacctgcttccctaccactcctcactgcattttccatacaaatgtttctattttattgttccttcttgtaataaagggaagataaaaccaaaaaaaaaaaaaaaaaaa

SEQ ID NO: 91 - protein, 370 aa, homo sapiens
MLGAVEGPRWKQAEDIRDIYDFRDVLGTGAFSEVILAEDKRTQKLVAIKCIAKEALEGKEGSMENEIAVLHKIKHPNIVALDDIYESGGHLYLIMQLVSGGELFDRIVEKGFYTERDASRLIFQVLDAVKYLHDLGIVHRDLKPENLLYYSLDEDSKIMISDFGLSKMEDPGSVLSTACGTPGYVAPEVLAQKPYSKAVDCWSIGVIAYILLCGYPPFYDENDAKLFEQILKAEYEFDSPYWDDISDSAKDFIRHLMEKDPEKRFTCEQALQHPWIAGDTALDKNIHQSVSEQIKKNFAKSKWKQAFNATAVVRHMRKLQLGTSQEGQGQTASHGELLTPVAGGPAAGCCCRDCCVEPGTELSPTLPHQL

SEQ ID NO: 92 - DNA, 2590 bp, homo sapiens
cctcttgcggccacaggcgcggcgtcctcggcggcgggcggcagctagcgggagccgggacgccggtgcagccgcagcgcgcggaggaacccgggtgtgccgggagctgggcggccacgtccggacgggaccgagacccctcgtagcgcattgcggcgacctcgccttccccggccgcgagcgcgccgctgcttgaaaagccgcggaacccaaggacttttctccggtccgagctcggggcgccccgcaggcgcacggtacccgtgctgcagtcgggcacgccgcggcgccgggggcctccgcagggcgatggagccggtctgcaaggaaagtgaggcgccgccgctgcgttctggaggaggggggcacaaggtctggagaccccgggtggcggacgggagccctccccccgccccgcctccggggcaccagctccggctccattgttcccgcccgggctggaggcgccgagcaccgagcgccgccgggagtcgagcgccggccgcggagctcttgcgaccccgccaggacccgaacagagcccgggggcggcgggccggagccggggacgcgggcacacgcccgctcgcacaagccacggcggactctcccgaggcggaacctccacgccgagcgagggtcagtttgaaaaggaggatcgagctcactgtggagtatccatggagatgtggagccttgtcaccaacctctaactgcagaactgggatgtggagctggaagtgcctcctcttctgggctgtgctggtcacagccacactctgcaccgctaggccgtccccgaccttgcctgaacaagatgctctcccctcctcggaggatgatgatgatgatgatgactcctcttcagaggagaaagaaacagataacaccaaaccaaaccgtatgcccgtagctccatattggacatccccagaaaagatggaaaagaaattgcatgcagtgccggctgccaagacagtgaagttcaaatgcccttccagtgggaccccaaaccccacactgcgctggttgaaaaatggcaaagaattcaaacctgaccacagaattggaggctacaaggtccgttatgccacctggagcatcataatggactctgtggtgccctctgacaagggcaactacacctgcattgtggagaatgagtacggcagcatcaaccacacataccagctggatgtcgtggagcggtcccctcaccggcccatcctgcaagcagggttgcccgccaacaaaacagtggccctgggtagcaacgtggagttcatgtgtaaggtgtacagtgacccgcagccgcacatccagtggctaaagcacatcgaggtgaatgggagcaagattggcccagacaacctgccttatgtccagatcttgaaggtaatcatggcaccagtcttcgtgggccagtctactgggaaggagaccactgtctcgggggctcaagttcctgtgggcaggctcagttgcccccgaatgggatcattcctcacgcttcaggcacacacactccatctcagtagggatctagccacatcccccaggactagtaacagaggtcacaaagtggaggtgagctgggaacagagggctgcagggatgggtggtgctggtctgtaataagctttgagagcaacgtcactggggctttggggtcagctacacaaggaaggcatttggacccctgccttttcattgcccgaaaccagagcctttccaccaagcgtttcccagtcttagccctgtgttctgagttacgtacgatctttctggcaaatggggtgcatgataagagcatctcttacgaagagttggaaaaacaaatgccatatataaattctaagccatatgaggacgaggagtaatggcattttcttcctttttcctctcactcccagacattcattgtccctgaatgctccattaatccagggaaggtaattgcctaaatctccagtggatctcgcaacaggaaggaaccagaagctgggaaagttgtttacctctttgtcccagagttagacctcatcctcccctagcttagctgtctcagagatatactggccctcccttctcttctctttgctgctggtgctaaaactgctctgtaggtcattggccactgtctccactcacaacccctgctccagtcctggagggagtgggttaaacacaaatagaacattccatttgaagcagtgattctttttttttttttttttttttaatcaaatgctttggacttttgaagtccacttgttctgtacttgtaaaagggaaagaaggccgggcgcagtcgtcacgcctgtaatcccagcactttagatcacttgaggtcaggagtttgagaccagcccggccaacatggtgaaaccccatctctactaaaaatacaaaaattagctgtgcatagtggttggcacctgtagtcccagctactcaggaggctgaggcaagctaactgcttgaacccagaaggcagaggttgcagtgagctgagatcacgccactgcactccagcctgggtgacagagtgagtgagactctgcgttaaaaaaaaaaaaaaaaaaaaaaa

SEQ ID NO: 93 - protein, 302 aa, homo sapiens
MWSWKCLLFWAVLVTATLCTARPSPTLPEQDALPSSEDDDDDDDSSSEEKETDNTKPNRMPVAPYWTSPEKMEKKLHAVPAAKTVKFKCPSSGTPNPTLRWLKNGKEFKPDHRIGGYKVRYATWSIIMDSVVPSDKGNYTCIVENEYGSINHTYQLDVVERSPHRPILQAGLPANKTVALGSNVEFMCKVYSDPQPHIQWLKHIEVNGSKIGPDNLPYVQILKVIMAPVFVGQSTGKETTVSGAQVPVGRLSCPRMGSFLTLQAHTLHLSRDLATSPRTSNRGHKVEVSWEQRAAGMGGAGL

SEQ ID NO: 94 - DNA, 1834 bp, homo sapiens
atttttagataatcattaaagaccacagaaaatgtaacagatcctactcttcaaaataattgctattcagtattaaaacgagcagtcagctgcgtgattcccgtgattgcgttacaagctttgtctccttcgacttggagtctttgtccaggacgatgagacactcaaagagaacttactgtcctgattgggatgacaaggattgggattatggaaaatggaggagcagcagcagtcataaaagaaggaagagatcacatagcagtgcccaggagaacaagcgctgcaaatacaatcactctaaaatgtgtgatagccattatttggaaagcaggtctataaatgagaaagattatcatagtcgacgctacattgatgagtacagaaatgactacactcaaggatgtgaacctggacatcgccaaagagaccatgaaagccggtatcagaaccatagtagcaagtcttctggtagaagtggaagaagtagttataaaagcaaacacaggattcaccacagtacttcacatcgtcgttcacatgggaagagtcaccgaaggaaaagaaccaggagtgtagaggatgatgaggagggtcacctgatctgtcagagtggagacgtactaagtgcaagatatgaaattgttgatactttaggtgaaggagcttttggaaaagttgtggagtgcatcgatcataaagcgggaggtagacatgtagcagtaaaaatagttaaaaatgtggatagatactgtgaagctgctcgctcagaaatacaagttctggaacatctgaatacaacagaccccaacagtactttccgctgtgtccagatgttggaatggtttgagcatcatggtcacatttgcattgtttttgaactattgggacttagtacttacgacttcattaaagaaaatggttttctaccatttcgactggatcatatcagaaagatggcatatcagatatgcaagtctgtgaattttttgcacagtaataagttgactcacacagacttaaagcctgaaaacatcttatttgtgcagtctgactacacagaggcgtataatcccaaaataaaacgtgatgaacgcaccttaataaatccagatattaaagttgtagactttggtagtgcaacatatgatgacgaacatcacagtacattggtatctacaagacattatagagcacctgaagttattttagccctagggtggtcccaaccatgtgatgtctggagcataggatgcattcttattgaatactatcttgggtttaccgtatttccaacacacgatagtaaggagcatttagcaatgatggaaaggattcttggacctctaccaaaacatatgatacagaaaaccaggaaacgtaaatattttcaccacgatcgattagactgggatgaacacagttctgccggcagatatgtttcaagagcctgtaaacctctgaaggaatttatgctttctcaagatgttgaacatgagcgtctctttgacctcattcagaaaatgttggagtatgatccagccaaaagaattactctcagagaagccttaaagcatcctttctttgaccttctgaagaaaagtatatagatctgtaattggacagctctctcgaagagatcttacagactgtatcagtctaatttttaaattttaagttattttgtacagctttgtaaattcttaacatttttatattgccatgtttattttgtttgggtaatttggttcattaagtacatagctaaggtaatgaacatctttttcagtaattgtaaagtgatttattcagaataaattttttgtgcttatga

SEQ ID NO: 95 - protein, 484 aa, homo sapiens
MRHSKRTYCPDWDDKDWDYGKWRSSSSHKRRKRSHSSAQENKRCKYNHSKMCDSHYLESRSINEKDYHSRRYIDEYRNDYTQGCEPGHRQRDHESRYQNHSSKSSGRSGRSSYKSKHRIHHSTSHRRSHGKSHRRKRTRSVEDDEEGHLICQSGDVLSARYEIVDTLGEGAFGKVVECIDHKAGGRHVAVKIVKNVDRYCEAARSEIQVLEHLNTTDPNSTFRCVQMLEWFEHHGHICIVFELLGLSTYDFIKENGFLPFRLDHIRKMAYQICKSVNFLHSNKLTHTDLKPENILFVQSDYTEAYNPKIKRDERTLINPDIKVVDFGSATYDDEHHSTLVSTRHYRAPEVILALGWSQPCDVWSIGCILIEYYLGFTVFPTHDSKEHLAMMERILGPLPKHMIQKTRKRKYFHHDRLDWDEHSSAGRYVSRACKPLKEFMLSQDVEHERLFDLIQKMLEYDPAKRITLREALKHPFFDLLKKSI

SEQ ID NO: 96 - DNA, 2352 bp, homo sapiens
ggcacgagggtctcaaaggctggctggagtggagccaaaggaaaagatcgttagagacagcgcccctgaccaaccacttagagcagcgcaggggtgggagggcggccgcaggctctcctctcgttagtgccccctgtgtttggggccccgtgatctcaacggtcctgccctcggtctccctcttcccccgccccgccctgggccaggtgttcgaatcccgactccagaactggcggcgtcccagtcccgcgggcgtggagcgccggaggacccgccctcgggctcatggcggccccggtccgcctgggccggaagcgcccgctgcctgcctgtcccaacccgctcttcgttcgctggctgaccgagtggcgggacgaggcgacccgcagcaggcaccgcacgcgcttcgtatttcagaaggcgctgcgttccctccgacggtacccactgccgctgcgcagcgggaaggaagctaagatcctacagcacttcggagacgggctctgccggatgctggacgagcggctgcagcggcaccgaacatcgggcggtgaccatgccccggactcaccatctggagagaacagtccagccccgcaggggcgacttgcggaagtccaggactcttccatgccagttcctgcccagcccaaagcgggaggctctggcagctactggccagctcggcactcaggagcccgagtgatactgctggtgctctaccgggagcacctgaatcctaatggtcaccacttcttaaccaaggaggagctgctgcagaggtgtgctcagaagtcccccagggtagcccctgggagtgccccaccctggccagccctccgctccctccttcacaggaacctggtcctcaggacacaccagccagccaggtactcattgaccccagagggcctggagctggcccagaagttggccgagtcagaaggcctgagcttgctgaatgtgggcatcgggcccaaggagccccctggggaggagacagcagtgccaggagcagcttcagcagagcttgccagtgaagcaggggtccagcagcagccactggagctgaggcctggagagtacagggtgctgttgtgtgtggacattggcgagacccgggggggcgggcacaggccggagctgctccgagagctacagcggctgcacgtgacccacacggtgcgcaagctgcacgttggagattttgtgtgggtggctcaggagaccaatcctagagacccagcaaaccctggggagttggtactggatcacattgtggagcgcaagcgactggatgacctttgcagcagcatcatcgacggccgcttccgggagcagaagttccgactgaagcgctgtggtctggagcgccgggtatacctggtggaagagcatggttccgtccacaacctcagccttcctgagagcacactgctgcaggctgtcaccaacactcaggtcattgatggcttttttgtgaagcgcacagcagacattaaggagtcagccgcctacctggccctcttgactcggggcctgcagagactctaccagggccacaccctacgcagccgcccctggggaacccctgggaaccctgaatcaggggccatgacctctccaaaccctctctgctcactcctcaccttcagtgacttcaacgcaggagccatcaagaataaggcccagtcggtgcgagaagtgtttgcccggcagctgatgcaggtgcgcggagtgagtggggagaaggcagcagccctggtggatcgatacagcacccctgccagcctcctggccgcctatgatgcctgtgccacccccaaggaacaagagacactgctgagcaccattaagtgtgggcgtctacagaggaatctggggcctgctctgagcaggaccttatcccagctctactgcagctacggccccttgacctgagcttatgccgtgaaacagcccccagcccccgtctgtcccccaacccaggctagccagccttttaacaacatcttttggggtacaattagaatctaagtgtttgcagccatatgtgtcatgtagaagatgcctagccctggggaccttgtgaaatacgcaggaaccagggataccatctggtccagtggtttttaaacaaagctgcttagcacctggaattccctggtcagggagatggagtcagtggggcattgcagcttggaatctattttatgtcaccagttggtcctcatcaaataaaatttccttaggagtgcagagggctcattgggaaaataaaaataataaaaataaataaaacttcctaaaagaaaagattgaaacccaaaaaaaaaaaaaaaaaaaaaaaaa

SEQ ID NO: 97 - protein, 476 aa, homo sapiens
MLDERLQRHRTSGGDHAPDSPSGENSPAPQGRLAEVQDSSMPVPAQPKAGGSGSYWPARHSGARVILLVLYREHLNPNGHHFLTKEELLQRCAQKSPRVAPGSAPPWPALRSLLHRNLVLRTHQPARYSLTPEGLELAQKLAESEGLSLLNVGIGPKEPPGEETAVPGAASAELASEAGVQQQPLELRPGEYRVLLCVDIGETRGGGHRPELLRELQRLHVTHTVRKLHVGDFVWVAQETNPRDPANPGELVLDHIVERKRLDDLCSSIIDGRFREQKFRLKRCGLERRVYLVEEHGSVHNLSLPESTLLQAVTNTQVIDGFFVKRTADIKESAAYLALLTRGLQRLYQGHTLRSRPWGTPGNPESGAMTSPNPLCSLLTFSDFNAGAIKNKAQSVREVFARQLMQVRGVSGEKAAALVDRYSTPASLLAAYDACATPKEQETLLSTIKCGRLQRNLGPALSRTLSQLYCSYGPLT

SEQ ID NO: 98 - DNA, 3166 bp, homo sapiens
aagagctccagagagaagtcgaggaagagagagacggggtcagagagagcgcgcgggcgtgcgagcagcgaaagcgacaggggcaaagtgagtgacctgcttttgggggtgaccgccggagcgcggcgtgagccctcccccttgggatcccgcagctgaccagtcgcgctgacggacagacagacagacaccgcccccagccccagttaccacctcctccccggccggcggcggacagtggacgcggcggcgagccgcgggcaggggccggagcccgcccccggaggcggggtggagggggtcggagctcgcggcgtcgcactgaaacttttcgtccaacttctgggctgttctcgcttcggaggagccgtggtccgcgcgggggaagccgagccgagcggagccgcgagaagtgctagctcgggccgggaggagccgcagccggaggagggggaggaggaagaagagaaggaagaggagagggggccgcagtggcgactcggcgctcggaagccgggctcatggacgggtgaggcggcggtgtgcgcagacagtgctccagcgcgcgcgctccccagccctggcccggcctcgggccgggaggaagagtagctcgccgaggcgccgaggagagcgggccgccccacagcccgagccggagagggacgcgagccgcgcgccccggtcgggcctccgaaaccatgaactttctgctgtcttgggtgcattggagccttgccttgctgctctacctccaccatgccaagtggtcccaggctgcacccatggcagaaggaggagggcagaatcatcacgaagtggtgaagttcatggatgtctatcagcgcagctactgccatccaatcgagaccctggtggacatcttccaggagtaccctgatgagatcgagtacatcttcaagccatcctgtgtgcccctgatgcgatgcgggggctgctccaatgacgagggcctggagtgtgtgcccactgaggagtccaacatcaccatgcagattatgcggatcaaacctcaccaaggccagcacataggagagatgagcttcctacagcacaacaaatgtgaatgcagaccaaagaaagatagagcaagacaagaaaatccctgtgggccttgctcagagcggagaaagcatttgtttgtacaagatccgcagacgtgtaaatgttcctgcaaaaacacacactcgcgttgcaaggcgaggcagcttgagttaaacgaacgtacttgcagatgtgacaagccgaggcggtgagccgggcaggaggaaggagcctccctcagggtttcgggaaccagatctctctccaggaaagactgatacagaacgatcgatacagaaaccacgctgccgccaccacaccatcaccatcgacagaacagtccttaatccagaaacctgaaatgaaggaagaggagactctgcgcagagcactttgggtccggagggcgagactccggcggaagcattcccgggcgggtgacccagcacggtccctcttggaattggattcgccattttatttttcttgctgctaaatcaccgagcccggaagattagagagttttatttctgggattcctgtagacacacccacccacatacatacatttatatatatatatattatatatatataaaaataaatatctctattttatatatataaaatatatatattctttttttaaattaacagtgctaatgttattggtgtcttcactggatgtatttgactgctgtggacttgagttgggaggggaatgttcccactcagatcctgacagggaagaggaggagatgagagactctggcatgatcttttttttgtcccacttggtggggccagggtcctctcccctgcccaagaatgtgcaaggccagggcatgggggcaaatatgacccagttttgggaacaccgacaaacccagccctggcgctgagcctctctaccccaggtcagacggacagaaagacaaatcacaggttccgggatgaggacaccggctctgaccaggagtttggggagcttcaggacattgctgtgctttggggattccctccacatgctgcacgcgcatctcgcccccaggggcactgcctggaagattcaggagcctgggcggccttcgcttactctcacctgcttctgagttgcccaggaggccactggcagatgtcccggcgaagagaagagacacattgttggaagaagcagcccatgacagcgccccttcctgggactcgccctcatcctcttcctgctccccttcctggggtgcagcctaaaaggacctatgtcctcacaccattgaaaccactagttctgtccccccaggaaacctggttgtgtgtgtgtgagtggttgaccttcctccatcccctggtccttcccttcccttcccgaggcacagagagacagggcaggatccacgtgcccattgtggaggcagagaaaagagaaagtgttttatatacggtacttatttaatatccctttttaattagaaattagaacagttaatttaattaaagagtagggttttttttcagtattcttggttaatatttaatttcaactatttatgagatgtatcttttgctctctcttgctctcttatttgtaccggtttttgtatataaaattcatgtttccaatctctctctccctgatcggtgacagtcactagcttatcttgaacagatatttaattttgctaacactcagctctgccctccccgatcccctggctccccagcacacattcctttgaaagagggtttcaatatacatctacatactatatatatattgggcaacttgtatttgtgtgtatatatatatatatatgtttatgtatatatgtgatcctgaaaaaataaacatcgctattctgttttttatatgttcaaaccaaacaagaaaaaatagagaattctacatactaaatctctctccttttttaattttaatatttgttatcatttatttattggtgctactgtttatccgtaataattgtggggaaaagatattaacatcacgtctttgtctctagtgcagtttttcgagatattccgtagtacatatttatttttaaacaacgacaaagaaatacagatatatcttaaaaaaaaaaaa

SEQ ID NO: 99 - protein, 191 aa, homo sapiens
MNFLLSWVHWSLALLLYLHHAKWSQAAPMAEGGGQNHHEVVKFMDVYQRSYCHPIETLVDIFQEYPDEIEYIFKPSCVPLMRCGGCSNDEGLECVPTEESNITMQIMRIKPHQGQHIGEMSFLQHNKCECRPKKDRARQENPCGPCSERRKHLFVQDPQTCKCSCKNTHSRCKARQLELNERTCRCDKPRR

SEQ ID NO: 100 - DNA, 1466 bp, homo sapiens
cacggccggagagacgcggaggaggagacatgagccggcgggcgcccagacggagcggccgtgacgctttcgcgctgcagccgcgcgccccgaccccggagcgctgacccctggccccacgcagctccgcgcccgggccggagagcgcaactcggcttccagacccgccgcgcatgctgtccccggactgagccgggcagccagcctcccacggacgcccggacggccggccggccagcagtgagcgagcttccccgcaccggccaggcgcctcctgcacagcggctgccgccccgcagcccctgcgccagcccggagggcgcagcgctcgggaggagccgcgcggggcgctgatgccgcagggcgcgccgcggagcgccccggagcagcagagtctgcagcagcagcagccggcgaggagggagcagcagcagcggcggcggcggcggcggcggcggcggaggcgcccggtcccggccgcgcggagcggacatgtgcaggctgggctaggagccgccgcctccctcccgcccagcgatgtattcagcgccctccgcctgcacttgcctgtgtttacacttcctgctgctgtgcttccaggtacaggtgctggttgccgaggagaacgtggacttccgcatccacgtggagaaccagacgcgggctcgggacgatgtgagccgtaagcagctgcggctgtaccagctctacagccggaccagtgggaaacacatccaggtcctgggccgcaggatcagtgcccgcggcgaggatggggacaagtatgcccagctcctagtggagacagacaccttcggtagtcaagtccggatcaagggcaaggagacggaattctacctgtgcatgaaccgcaaaggcaagctcgtggggaagcccgatggcaccagcaaggagtgtgtgttcatcgagaaggttctggagaacaactacacggccctgatgtcggctaagtactccggctggtacgtgggcttcaccaagaaggggcggccgcggaagggccccaagacccgggagaaccagcaggacgtgcatttcatgaagcgctaccccaaggggcagccggagcttcagaagcccttcaagtacacgacggtgaccaagaggtcccgtcggatccggcccacacaccctgcctaggccaccccgccgcggccctcaggtcgccctggccacactcacactcccagaaaactgcatcagaggaatatttttacatgaaaaataaggattttattgttgacttgaaacccccgatgacaaaagactcacgcaaagggactgtagtcaacccacaggtgcttgtctctctctaggaacagacaactctaaactcgtccccagaggaggacttgaatgaggaaaccaacactttgagaaaccaaagtcctttttcccaaaggttctgaaaggaaaaaaaaaaaaaaacaaaaaaaaaaaaaaaa

SEQ ID NO: 101 - protein, 207 aa, homo sapiens
MYSAPSACTCLCLHFLLLCFQVQVLVAEENVDFRIHVENQTRARDDVSRKQLRLYQLYSRTSGKHIQVLGRRISARGEDGDKYAQLLVETDTFGSQVRIKGKETEFYLCMNRKGKLVGKPDGTSKECVFIEKVLENNYTALMSAKYSGWYVGFTKKGRPRKGPKTRENQQDVHFMKRYPKGQPELQKPFKYTTVTKRSRRIRPTHPA

SEQ ID NO: 102 - DNA, 2926 bp, homo sapiens
cggaagcggaagtcggggggcgcgccagctcgtagcaggggagcgcccgcggcgtcgggtttgggctggaggtcgccatggggcgaggcagcggcaccttcgagcgtctcctagacaaggcgaccagccagctcctgttggagacagattgggagtccattttgcagatctgcgacctgatccgccaaggggacacacaagcaaaatatgctgtgaattccatcaagaagaaagtcaacgacaagaacccacacgtcgccttgtatgccctggaggtcatggaatctgtggtaaagaactgtggccagacagttcatgatgaggtggccaacaagcagaccatggaggagctgaaggacctgctgaagagacaagtggaggtaaacgtccgtaacaagatcctgtacctgatccaggcctgggcgcatgccttccggaacgagcccaagtacaaggtggtccaggacacctaccagatcatgaaggtggaggggcacgtctttccagaattcaaagagagcgatgccatgtttgctgccgagagagccccagactgggtggacgctgaggaatgccaccgctgcagggtgcagttcggggtgatgacccgtaagcaccactgccgggcgtgtgggcagatattctgtggaaagtgttcttccaagtactccaccatccccaagtttggcatcgagaaggaggtgcgcgtgtgtgagccctgctacgagcagctgaacaggaaagcggagggaaaggccacttccaccactgagctgccccccgagtacctgaccagccccctgtctcagcagtcccagctgccccccaagagggacgagacggccctgcaggaggaggaggagctgcagctggccctggcgctgtcacagtcagaggcggaggagaaggagaggctgagacagaagtccacgtacacttcgtaccccaaggcggagcccatgccctcggcctcctcagcgccccccgccagcagcctgtactcttcacctgtgaactcgtcggcgcctctggctgaggacatcgaccctgagctcgcacggtatctcaaccggaactactgggagaagaagcaggaggaggctcgcaagagccccacgccatctgcgcccgtgcccctgacggagccggctgcacagcctggggaagggcacgcagcccccaccaacgtggtggagaaccccctcccggagacagactctcagcccattcctccctctggtggcccctttagtgagccacagttccacaatggcgagtctgaggagagccacgagcagttcctgaaggcgctgcagaacgccgtcaccaccttcgtgaaccgcatgaagagtaaccacatgcggggccgcagcatcaccaatgactcggccgtgctctcactcttccagtccatcaacggcatgcacccgcagctgctggagctgctcaaccagctggacgagcgcaggctgtactatgaggggctgcaggacaagctggcacagatccgcgatgcccggggggcgctgagtgccctgcgcgaagagcaccgggagaagcttcgccgggcagccgaggaggcagagcgccagcgccagatccagctggcccagaagctggagataatgcggcagaagaagcaggagtacctggaggtgcagaggcagctggccatccagcgcctgcaggagcaggagaaggagcggcagatgcggctggagcagcagaagcagacggtccagatgcgcgcgcagatgcccgccttccccctgccctacgcccagctccaggccatgcccgcagccggaggtgtgctctaccagccctcgggaccagccagcttccccagcaccttcagccctgccggctcggtggagggctccccaatgcacggcgtgtacatgagccagccggcccctgccgctggcccctaccccagcatgcccagcactgcggctgatcccagcatggtgagtgcctacatgtacccagcaggggccactggggcgcaggcggccccccaggcccaggccggacccaccgccagccccgcttactcatcctaccagcctactcccacagcgggctaccagaacgtggcctcccaggccccacagagcctcccggccatctctcagcctccgcagtccagcaccatgggctacatggggagccagtcagtctccatgggctaccagccttacaacatgcagaatctcatgaccaccctcccaagccaggatgcgtctctgccaccccagcagccctacatcgcggggcagcagcccatgtaccagcagatggcaccctctggcggtcccccccagcagcagccccccgtggcccagcaaccgcaggcacaggggccgccggcacagggcagcgaggcccagctcatttcattcgactgacccaggccatgctcacgtccggagtaacactacatacagttcacctgaaacgcctcgtctctaactgccgtcgtcctgcctccctgtcctctactgccggtagtgtcccttctctgcgagtgagggggggccttcaccccaagcccacctcccttgtcctcagcctactgcagtccctgagttagtctctgctttctttccccagggctgggccatggggagggaaggactttctcccaggggaagcccccagccctgtgggtcatggtctgtgagaggtggcaggaatggggaccctcaccccccaagcagcctgtgccctctggccgcactgtgagctggctgtggtgtctgggtgtggcctggggctccctctgcaggggcctctctcggcagccacagccaagggtggaggcttcaggtctccagcttctctgcttctcagctgccatctccagtgccccagaatggtacagcgataataaaatgtatttcagaaaaaaaaaaaaaaaaaa

SEQ ID NO: 103 - protein, 777 aa, homo sapiens
MGRGSGTFERLLDKATSQLLLETDWESILQICDLIRQGDTQAKYAVNSIKKKVNDKNPHVALYALEVMESVVKNCGQTVHDEVANKQTMEELKDLLKRQVEVNVRNKILYLIQAWAHAFRNEPKYKVVQDTYQIMKVEGHVFPEFKESDAMFAAERAPDWVDAEECHRCRVQFGVMTRKHHCRACGQIFCGKCSSKYSTIPKFGIEKEVRVCEPCYEQLNRKAEGKATSTTELPPEYLTSPLSQQSQLPPKRDETALQEEEELQLALALSQSEAEEKERLRQKSTYTSYPKAEPMPSASSAPPASSLYSSPVNSSAPLAEDIDPELARYLNRNYWEKKQEEARKSPTPSAPVPLTEPAAQPGEGHAAPTNVVENPLPETDSQPIPPSGGPFSEPQFHNGESEESHEQFLKALQNAVTTFVNRMKSNHMRGRSITNDSAVLSLFQSINGMHPQLLELLNQLDERRLYYEGLQDKLAQIRDARGALSALREEHREKLRRAAEEAERQRQIQLAQKLEIMRQKKQEYLEVQRQLAIQRLQEQEKERQMRLEQQKQTVQMRAQMPAFPLPYAQLQAMPAAGGVLYQPSGPASFPSTFSPAGSVEGSPMHGVYMSQPAPAAGPYPSMPSTAADPSMVSAYMYPAGATGAQAAPQAQAGPTASPAYSSYQPTPTAGYQNVASQAPQSLPAISQPPQSSTMGYMGSQSVSMGYQPYNMQNLMTTLPSQDASLPPQQPYIAGQQPMYQQMAPSGGPPQQQPPVAQQPQAQGPPAQGSEAQLISFD

SEQ ID NO: 104 - protein, 540 aa, homo sapiens
MNGEAICSALPTIPYHKLADLRYLSRGASGTVSSARHADWRVQVAVKHLHIHTPLLDSERKDVLREAEILHKARFSYILPILGICNEPEFLGIVTEYMPNGSLNELLHRKTEYPDVAWPLRFRILHEIALGVNYLHNMTPPLLHHDLKTQNILLDNEFHVKIADFGLSKWRMMSLSQSRSSKSAPEGGTIIYMPPENYEPGQKSRASIKHDIYSYAVITWEVLSRKQPFEDVTNPLQIMYSVSQGHRPVINEESLPYDIPHRARMISLIESGWAQNPDERPSFLKCLIELEPVLRTFEEITFLEAVIQLKKTKLQSVSSAIHLCDKKKMELSLNIPVNHGPQEESCGSSQLHENSGSPETSRSLPAPQDNDFLSRKAQDCYFMKLHHCPGNHSWDSTISGSQRAAFCDHKTTPCSSAIINPLSTAGNSERLQPGIAQQWIQSKREDIVNQMTEACLNQSLDALLSRDLIMKEDYELVSTKPTRTSKVRQLLDTTDIQGEEFAKVIVQKLKDNKQMGLQPYPEILVVSRSPSLNLLQNKSM

SEQ ID NO: 105 - DNA, 3571 bp, homo sapiens
cacagcttgggactgctgaggggcaggcggctgcaggctaggggcggctcggagtccgctggccacccagctgagaggagaggcgcccccggggacgcactgagattatgaggctctggcctccactggccactcactcgtgaccctttccaccacggcggagccttccaagcctacctcctgccgtgtggtgatctacctgcagcgggagatgtcgggggacacctgcctgtgcccagcctcaggggccaagcccaagctcagtggcttcaagggaggagggttgggcaacaagtatgtccagctcaacgtgggcggctctctgtactacaccactgtgcgggccctgacccgccacgacaccatgctcaaggccatgttcagtgggcgcatggaggtgctgaccgacaaagaaggctggatcctcatagaccgttgtggaaagcactttggcaccattttgaattacctccgagatgacaccatcaccctccctcagaaccggcaagaaatcaaggaattgatggctgaagcaaagtattacctcatccaggggctggtgaatatgtgccagagtgccctgcaggacaagaaggactcctaccagcctgtgtgcaacatccccatcatcacatccctaaaggaggaggagcggctcatcgaatcctccaccaagcccgtggtgaagctgctgtacaacagaagcaacaacaagtattcctacaccagcaactctgacgaccacctgctgaaaaacatcgagctgtttgacaagctctccctgcgcttcaacggccgcgtgctcttcatcaaggatgtcattggtgacgagatctgctgctggtccttttatggccagggccgtaagctggcagaggtgtgctgtacctccatcgtgtatgccacggagaagaagcagaccaaggtggaattcccagaggcccgaatctatgaggagacactcaacgtcctactctatgagactccccgcgtccccgacaactccttgttggaggccacaagccgtagccgcagccaggcttcccccagtgaagatgaggagacctttgaactgcgggaccgtgtccgccgcatccacgtcaagcgctacagcacttacgatgaccggcagctcggccaccagtctacccatcgcgactgaccagaccctcagggagtcagggcacgggaggccctatctcccatcctgtggaacccgccccattggccaccccatgctgctgctgcctgggtctctgctctagcacccagaggcatgacaggccctgctcagaggtcagagggtctgggcagaggagggaccacattcccctgccttgcccctgagcacttctggagactgcgtcctgtcctatctgctcaccatcacccttcctgcccgacggagctgcttctgctccctggggcatatggactgacccacctcctgctgagaaccttcccctaggccctgtgcagaagggctactgccccttaggcctcagctgggggaaaggcagttctggtgctgtagaggccctggtgcagaaagtgggacgtcttttttcctaaggtgtttaagcacaggcttgataagtttggtttttaaaaaataatctaggaaatgaataattctaaatctagtaatgaggaaactgagcatttcttttgccctccagggtgccaagaccctacatatgacagaacccttggcccttctccatgcctgtgggatctgtttctttaaagcactttgtactgttattcaggaggttgataatctccttgacccatgtctttctaccctaatccccacttccctgcagaatcaatctgagggaggggataaagaggaagcaataaaaaaaaaacatccgacagagcagctctggctttgccagcctggccagcagctcagagtgcaccgaggagggaaggatggctaagctgggaccggcagtcctcacagggtgcctgtgagaaaggacattttacccccacatcatagtcacatcactgactcctaggtctagcacgactgctctttgtgattctcttgagtacccttggcttccagccatgctgtcctcacatacggtaaagccaaagagctgtcacatgggccagaaacatgagccacggcaggaagaccgtggagcccgtgggcactgcatggtgttggctggcatgcccatcagctgaggacagcaaactcccagcagcccctacagaggtggcacatgcttggccacacatctactccctgcccacaccatctatgctcttggttggtgctggctgggatggcggttctgcccagtggtgtctctgagcgcgggatgacaggagcaaccgaagcaccctgaaggccttcactccttgttgggtaactcagccatggagatgccaagcactagccaggaggtgagttcctctttagggctttggttttcattcctttttgtttggcttggccaaaccagaattcagcttatctgaattattttccaaaggaatgctgtcagggagggactgttctgccagcctaacaaagcaacgtagccacgtatagtacccactttctgctctttggagagaacacaggttatcaagttcatctctcttgactactcttatgatagctgatgccacagagcctatgggcaaatgccagacccagggttagacacaaggacctgaagtgacatgacggcgggacaggggaaatgtgactttctaattaggcattttatgttagtcacagtcttgaatgtataaacagcactaagactctcaggtcaggtaccttggtgatcagctactagttcttccagccctcattgaggtaacaagataaagacaaatccacttctttggccaaattcaggctttggctttatgactttcccacagagactggaatgcgtcagcctgagaccactggcctattttctcagctgccctcttgaggtcctttaacactcaaattcccagctccccactgaggtgttgtgatgcttgccttttgacctccccatcccctttagtccctgcttactactttgacattcacatcctcagtgtctcagtcttttttgccgagaaagcacagtagtctgggactgggcatttatcttctctgactgaaaatctctccttggtcttaaggaaaatactaacattgaactcactgacatgatcttagcttctttaatcagactttgtgacttaaaagtttgggggttttctttgaaagtttccagccctattcagaaagcaactcttggctgtgtgcatttttcaactccaagcagcccaggggtaagtaaacaaagtatggatgaaggtcagattttcttgtcagtttctgagaaacctggcagcctgctgttaacaacacaggccagtattgggttttattgaatttggtatgtgaccaaggtcggcctaaaggatggcgcaggtcctgggcaggaaagaatttttcctttatcacataactgtaatatttggttgctcagcataagtgatggaagcaaacactaatttctaataaaattgtgttaaactc

SEQ ID NO: 106 - protein, 316 aa, homo sapiens
MSGDTCLCPASGAKPKLSGFKGGGLGNKYVQLNVGGSLYYTTVRALTRHDTMLKAMFSGRMEVLTDKEGWILIDRCGKHFGTILNYLRDDTITLPQNRQEIKELMAEAKYYLIQGLVNMCQSALQDKKDSYQPVCNIPIITSLKEEERLIESSTKPVVKLLYNRSNNKYSYTSNSDDHLLKNIELFDKLSLRFNGRVLFIKDVIGDEICCWSFYGQGRKLAEVCCTSIVYATEKKQTKVEFPEARIYEETLNVLLYETPRVPDNSLLEATSRSRSQASPSEDEETFELRDRVRRIHVKRYSTYDDRQLGHQSTHRD

SEQ ID NO: 107 - DNA, 1762 bp, homo sapiens
tggggcactggtacctccaggacctggagtgtactggaagaaatggtgcagtccagatgcatcactgtaagcgataccgctcccctgaaccagacccgtacctgagctaccgatggaagaggaggaggtcctacagtcgggaacatgaagggagactgcgatacccgtcccgaagggagcctcccccacgaagatctcggtccagaagccatgaccgcctgccctaccagaggaggtaccgggagcgccgtgacagcgatacataccggtgtgaagagcggagcccatcctttggagaggactactatggaccttcacgttctcgtcatcgtcggcgatcgcgggagagggggccataccggacccgcaagcatgcccaccactgccacaaacgccgcaccaggtcttgtagcagcgcctcctcgagaagccaacagagcagtaagcgcacaggccggagtgtggaagatgacaaggagggtcacctggtgtgccggatcggcgattggctccaagagcgatatgagattgtggggaacctgggtgaaggcacctttggcaaggtggtggagtgcttggaccatgccagagggaagtctcaggttgccctgaagatcatccgcaacgtgggcaagtaccgggaggctgcccggctagaaatcaacgtgctcaaaaaaatcaaggagaaggacaaagaaaacaagttcctgtgtgtcttgatgtctgactggttcaacttccacggtcacatgtgcatcgcctttgagctcctgggcaagaacacctttgagttcctgaaggagaataacttccagccttaccccctaccacatgtccggcacatggcctaccagctctgccacgcccttagatttctgcatgagaatcagctgacccatacagacttgaaacctgagaacatcctgtttgtgaattctgagtttgaaaccctctacaatgagcacaagagctgtgaggagaagtcagtgaagaacaccagcatccgagtggctgactttggcagtgccacatttgaccatgagcaccacaccaccattgtggccacccgtcactatcgcccgcctgaggtgatccttgagctgggctgggcacagccctgtgacgtctggagcattggctgcattctctttgagtactaccggggcttcacactcttccagacccacgaaaaccgagagcacctggtgatgatggagaagatcctagggcccatcccatcacacatgatccaccgtaccaggaagcagaaatatttctacaaagggggcctagtttgggatgagaacagctctgacggccggtatgtgaaggagaactgcaaacctctgaagagttacatgctccaagactccctggagcacgtgcagctgtttgacctgatgaggaggatgttagaatttgaccctgcccagcgcatcacactggccgaggccctgctgcaccccttctttgctggcctgacccctgaggagcggtccttccacaccagccgcaacccaagcagatgacaggcacaggccaccgcatgaggagatggagggcgggactgggccgcccagccccttgactccagcctcgaccgccagccccaggccagagccacccaatgaacagtgcaatgtgaaggaaggcaggagcctgcaggggagcagacttggtgcccagctgccagaaagcacagatttgacccaagctatttatatgttataaagttataataaagtgtttcttactgtttgta

SEQ ID NO: 108 - protein, 490 aa, homo sapiens
MHHCKRYRSPEPDPYLSYRWKRRRSYSREHEGRLRYPSRREPPPRRSRSRSHDRLPYQRRYRERRDSDTYRCEERSPSFGEDYYGPSRSRHRRRSRERGPYRTRKHAHHCHKRRTRSCSSASSRSQQSSKRTGRSVEDDKEGHLVCRIGDWLQERYEIVGNLGEGTFGKVVECLDHARGKSQVALKIIRNVGKYREAARLEINVLKKIKEKDKENKFLCVLMSDWFNFHGHMCIAFELLGKNTFEFLKENNFQPYPLPHVRHMAYQLCHALRFLHENQLTHTDLKPENILFVNSEFETLYNEHKSCEEKSVKNTSIRVADFGSATFDHEHHTTIVATRHYRPPEVILELGWAQPCDVWSIGCILFEYYRGFTLFQTHENREHLVMMEKILGPIPSHMIHRTRKQKYFYKGGLVWDENSSDGRYVKENCKPLKSYMLQDSLEHVQLFDLMRRMLEFDPAQRITLAEALLHPFFAGLTPEERSFHTSRNPSR

SEQ ID NO: 109 - DNA, 3674 bp, homo sapiens
gcgggaagcaccatgcagtttgtatcctgggccacactgctaacgctcctggtgcgggacctggccgagatggggagcccagacgccgcggcggccgtacgcaaggacaggctgcacccgaggcaagtgaaattattagagaccctgggcgaatacgaaatcgtgtctcccatccgagtgaacgctctcggagaaccctttcccacgaacgtccacttcaaaagaacgcgacggagcattaactctgccactgacccctggcctgccttcgcctcctcctcttcctcctctacctcctcccaggcgcattaccgcctctctgccttcggccagcagtttctatttaatctcaccgccaatgccggatttatcgctccactgttcactgtcaccctcctcgggacgcccggggtgaatcagaccaagttttattccgaagaggaagcggaactcaagcactgtttctacaaaggctatgtcaataccaactccgagcacacggccgtcatcagcctctgctcaggaatgctgggcacattccggtctcatgatggggattattttattgaaccactacagtctatggatgaacaagaagatgaagaggaacaaaacaaaccccacatcatttataggcgcagcgccccccagagagagccctcaacaggaaggcatgcatgtgacacctcagaacacaaaaataggcacagtaaagacaagaagaaaaccagagcaagaaaatggggagaaaggattaacctggctggtgacgtagcagcattaaacagcggcttagcaacagaggcattttctgcttatggtaataagacggacaacacaagagaaaagaggacccacagaaggacaaaacgttttttatcctatccacggtttgtagaagtcttggtggtggcagacaacagaatggtttcataccatggagaaaaccttcaacactatattttaactttaatgtcaattgtagcctctatctataaagacccaagtattggaaatttaattaatattgttattgtgaacttaattgtgattcataatgaacaggatgggccttccatatcttttaatgctcagacaacattaaaaaacctttgccagtggcagcattcgaagaacagtccaggtggaatccatcatgatactgctgttctcttaacaagacaggatatctgcagagctcacgacaaatgtgataccttaggcctggctgaactgggaaccatttgtgatccctatagaagctgttctattagtgaagatagtggattgagtacagcttttacgatcgcccatgagctgggccatgtgtttaacatgcctcatgatgacaacaacaaatgtaaagaagaaggagttaagagtccccagcatgtcatggctccaacactgaacttctacaccaacccctggatgtggtcaaagtgtagtcgaaaatatatcactgagtttttagacactggttatggcgagtgtttgcttaacgaacctgaatccagaccctaccctttgcctgtccaactgccaggcatcctttacaacgtgaataaacaatgtgaattgatttttggaccaggttctcaggtgtgcccatatatgatgcagtgcagacggctctggtgcaataacgtcaatggagtacacaaaggctgccggactcagcacacaccctgggccgatgggacggagtgcgagcctggaaagcactgcaagtatggattttgtgttcccaaagaaatggatgtccccgtgacagatggatcctggggaagttggagtccctttggaacctgctccagaacatgtggagggggcatcaaaacagccattcgagagtgcaacagaccagaaccaaaaaatggtggaaaatactgtgtaggacgtagaatgaaatttaagtcctgcaacacggagccatgtctcaagcagaagcgagacttccgagatgaacagtgtgctcactttgacgggaagcattttaacatcaacggtctgcttcccaatgtgcgctgggtccctaaatacagtggaattctgatgaaggaccggtgcaagttgttctgcagagtggcagggaacacagcctactatcagcttcgagacagagtgatagatggaactccttgtggccaggacacaaatgatatctgtgtccagggcctttgccggcaagctggatgcgatcatgttttaaactcaaaagcccggagagataaatgtggggtttgtggtggcgataattcttcatgcaaaacagtggcaggaacatttaatacagtacattatggttacaatactgtggtccgaattccagctggtgctaccaatattgatgtgcggcagcacagtttctcaggggaaacagacgatgacaactacttagctttatcaagcagtaaaggtgaattcttgctaaatggaaactttgttgtcacaatggccaaaagggaaattcgcattgggaatgctgtggtagagtacagtgggtccgagactgccgtagaaagaattaactcaacagatcgcattgagcaagaacttttgcttcaggttttgtcggtgggaaagttgtacaaccccgatgtacgctattctttcaatattccaattgaagataaacctcagcagttttactggaacagtcatgggccatggcaagcatgcagtaaaccctgccaaggggaacggaaacgaaaacttgtttgcaccagggaatctgatcagcttactgtttctgatcaaagatgcgatcggctgccccagcctggacacattactgaaccctgtggtacagactgtgacctgaggtggcatgttgccagcaggagtgaatgtagtgcccagtgtggcttgggttaccgcacattggacatctactgtgccaaatatagcaggctggatgggaagactgagaaggttgatgatggtttttgcagcagccatcccaaaccaagcaaccgtgaaaaatgctcaggggaatgtaacacgggtggctggcgctattctgcctggactgaatgttcaaaaagctgtgacggtgggacccagaggagaagggctatttgtgtcaatacccgaaatgatgtactggatgacagcaaatgcacacatcaagagaaagttaccattcagaggtgcagtgagttcccttgtccacagtggaaatctggagactggtcagaggtaagatgggagggctgttatttcccctaggtcatctcttacattctagttctggtgctctctatctgtttaagacaaacccttgtgcacctttctcccacctctccctttctcccttgtctcccttgagaaaacaactccagttctctgcctgcaccatgactgtcgtactggatgtaactagtctaccagtgacctcagggcactttgggcttggctagatcactcactgttgtagcttctgttgtgattttgaagttgcagtccatcaccttccctcctctttgagccctagctaagtcactgaaaggaaatcatggatttattaatcataaagctatactagctcacatctgaagtcaacatgaagtttcctacttccttgtctttgaaataagagaattagaccccagggagtgacctctctgacttacccatccaactgcccaaaaaaaaaaaaaaaaaaaaaaaaa

SEQ ID NO: 110 - protein, 1072 aa, homo sapiens
MQFVSWATLLTLLVRDLAEMGSPDAAAAVRKDRLHPRQVKLLETLGEYEIVSPIRVNALGEPFPTNVHFKRTRRSINSATDPWPAFASSSSSSTSSQAHYRLSAFGQQFLFNLTANAGFIAPLFTVTLLGTPGVNQTKFYSEEEAELKHCFYKGYVNTNSEHTAVISLCSGMLGTFRSHDGDYFIEPLQSMDEQEDEEEQNKPHIIYRRSAPQREPSTGRHACDTSEHKNRHSKDKKKTRARKWGERINLAGDVAALNSGLATEAFSAYGNKTDNTREKRTHRRTKRFLSYPRFVEVLVVADNRMVSYHGENLQHYILTLMSIVASIYKDPSIGNLINIVIVNLIVIHNEQDGPSISFNAQTTLKNLCQWQHSKNSPGGIHHDTAVLLTRQDICRAHDKCDTLGLAELGTICDPYRSCSISEDSGLSTAFTIAHELGHVFNMPHDDNNKCKEEGVKSPQHVMAPTLNFYTNPWMWSKCSRKYITEFLDTGYGECLLNEPESRPYPLPVQLPGILYNVNKQCELIFGPGSQVCPYMMQCRRLWCNNVNGVHKGCRTQHTPWADGTECEPGKHCKYGFCVPKEMDVPVTDGSWGSWSPFGTCSRTCGGGIKTAIRECNRPEPKNGGKYCVGRRMKFKSCNTEPCLKQKRDFRDEQCAHFDGKHFNINGLLPNVRWVPKYSGILMKDRCKLFCRVAGNTAYYQLRDRVIDGTPCGQDTNDICVQGLCRQAGCDHVLNSKARRDKCGVCGGDNSSCKTVAGTFNTVHYGYNTVVRIPAGATNIDVRQHSFSGETDDDNYLALSSSKGEFLLNGNFVVTMAKREIRIGNAVVEYSGSETAVERINSTDRIEQELLLQVLSVGKLYNPDVRYSFNIPIEDKPQQFYWNSHGPWQACSKPCQGERKRKLVCTRESDQLTVSDQRCDRLPQPGHITEPCGTDCDLRWHVASRSECSAQCGLGYRTLDIYCAKYSRLDGKTEKVDDGFCSSHPKPSNREKCSGECNTGGWRYSAWTECSKSCDGGTQRRRAICVNTRNDVLDDSKCTHQEKVTIQRCSEFPCPQWKSGDWSEVRWEGCYFP

SEQ ID NO: 111 - DNA, 2104 bp, homo sapiens
ctctgccgagcctccttaaaactctgccgttaaaatgggggcgggtttttcaactcaaaaagcgctcaatttttttcttttcaaaaaaagctgatgaggtcggaaaaaagggagaagaaaccggcaccctctctgagaggcaacagaagcagcaattgtttcagcgaaaaaagcagcaagggagggagtgaaggaaaaaagcaaaaaagggggcgacacgcaagtgcctgtaggggtgaaaggagcagggaccggcgatctagggggggatcagctacaaaagaaactgtcactgggagcggtgcggccaaggaggaagcagtgctgccaggctctgctccagggcacagctggctggcggctgccctgtccgcagcaaaggggcacaggccggggaccgcgagaggtggcaaagtggcaccgggcgccgaggctgctgagcgctcgccgagacggcgaccggactggctgccccggaactgcggcgactctccctactcagaacttggcctacgtttcccaggactctccccatctccagaggcccccacaaaaccgggaaaggaaggaaaggacagcggcggcagcagctcaatgagtgcctacagcagaaagcctgaacgagctcggtcgtaggcgggaagttcccgggggggctgcccagtgcagccgcaatgctgccgcgagctgccccagcagtccgggctccgtagacgctttccgcatcactctccttcctcgggctgccgggagtcccgggacctggcggggccggcatgacgggcttctcgggggcccgccgcacgcccggcagcctccggagacgcgcgccgagcccggctcccacggcctctgaggctcggcggggctgcggctgcctggcgggcgggctccggagctttcctgagcggcattagcccacggcttggcccggacgcgaccaaaggctcttctggagaagcccagagcactgggcaatcgttacgacctgtaacttgagggccaccgaactgctactcccgttcgcctttggcgatcatcttttaaccctccggagcacgtcagcatccagccaccgcggcgctctcccagcagcggaggacccaggactatcccttcggcgagacggatggaaaccgagccccctggaggacctgcccctgcagttctgcctcacacggctcaagtcaccaccgtgaacaagggaccctaaagaatggccgagccttgggggaacgagttggcgtccgcagctgccaggggggacctagagcaacttactagtttgttgcaaaataatgtaaacgtcaatgcacaaaatggatttggaaggactgcgctgcaggttatgaaacttggaaatcccgagattgccaggagactgctacttagaggtgctaatcccgatttgaaagaccgaactggtttcgctgtcattcatgatgcggccagagcaggtttcctggacactttacagactttgctggagtttcaagctgatgttaacatcgaggataatgaagggaacctgcccttgcacttggctgccaaagaaggccacctccgggtggtggagttcctggtgaagcacacggccagcaatgtggggcatcggaaccataagggggacaccgcctgtgatttggccaggctctatgggaggaatgaggttgttagcctgatgcaggcaaacggggctgggggagccacaaatcttcaataaacgtggggagggctcccccacgttgcctctactttatcaattaactgagtagctctcctgacttttaatgtcatttgttaaaatacagttctgtcatatgttaagcagctaaattttctgaaactgcataagtgaaaatcttacaacaggcttatgaatatatttaagcaacatctttttaacctgcaaaatctgttctaacatgtaattgcagataactttgactttcttctgaatattttatctttccttggcttttcccttgcttccccttttgccaatctcaacacccaagttgaagactttgtttttaaaatggtttgtcctgatgcttttgtctaattaaaacactttcaaaacaggaaaaaaaaaaaaaaaaaa

SEQ ID NO: 112 - protein, 168 aa, homo sapiens
MAEPWGNELASAAARGDLEQLTSLLQNNVNVNAQNGFGRTALQVMKLGNPEIARRLLLRGANPDLKDRTGFAVIHDAARAGFLDTLQTLLEFQADVNIEDNEGNLPLHLAAKEGHLRVVEFLVKHTASNVGHRNHKGDTACDLARLYGRNEVVSLMQANGAGGATNLQ

SEQ ID NO: 113 - DNA, 2650 bp, homo sapiens
gccgcgctggtggcggcggcgcgtcgttgcagttgcgccatctgtcaggagcggagccggcgaggagggggctgccgcgggcgaggaggaggggtcgccgcgagccgaaggccttcgagacccgcccgccgcccggcggcgagagtagaggcgaggttgttgtgcgagcggcgcgtcctctcccgcccgggcgcgccgcgcttctcccagcgcaccgaggaccgcccgggcgcacacaaagccgccgcccgcgccgcaccgcccggcggccgccgcccgcgccagggagggattcggccgccgggccggggacaccccggcgccgccccctcggtgctctcggaaggcccaccggctcccgggcccgccggggaccccccggagccgcctcggccgcgccggaggagggcggggagaggaccatgtgagtgggctccggagcctcagcgccgcgcagtttttttgaagaagcaggatgctgatctaaacgtggaaaaagaccagtcctgcctctgttgtagaagacatgtggtgtatataaagtttgtgatcgttggcggacattttggaatttagataatgggctgtgtgcaatgtaaggataaagaagcaacaaaactgacggaggagagggacggcagcctgaaccagagctctgggtaccgctatggcacagaccccacccctcagcactaccccagcttcggtgtgacctccatccccaactacaacaacttccacgcagccgggggccaaggactcaccgtctttggaggtgtgaactcttcgtctcatacggggaccttgcgtacgagaggaggaacaggagtgacactctttgtggccctttatgactatgaagcacggacagaagatgacctgagttttcacaaaggagaaaaatttcaaatattgaacagctcggaaggagattggtgggaagcccgctccttgacaactggagagacaggttacattcccagcaattatgtggctccagttgactctatccaggcagaagagtggtactttggaaaacttggccgaaaagatgctgagcgacagctattgtcctttggaaacccaagaggtacctttcttatccgcgagagtgaaaccaccaaaggtgcctattcactttctatccgtgattgggatgatatgaaaggagaccatgtcaaacattataaaattcgcaaacttgacaatggtggatactacattaccacccgggcccagtttgaaacacttcagcagcttgtacaacattactcagagagagctgcaggtctctgctgccgcctagtagttccctgtcacaaagggatgccaaggcttaccgatctgtctgtcaaaaccaaagatgtctgggaaatccctcgagaatccctgcagttgatcaagagactgggaaatgggcagtttggggaagtatggatgggtacctggaatggaaacacaaaagtagccataaagactcttaaaccaggcacaatgtcccccgaatcattccttgaggaagcgcagatcatgaagaagctgaagcacgacaagctggtccagctctatgcagtggtgtctgaggagcccatctacatcgtcaccgagtatatgaacaaaggaagtttactggatttcttaaaagatggagaaggaagagctctgaaattaccaaatcttgtggacatggcagcacaggtggctgcaggaatggcttacatcgagcgcatgaattatatccatagagatctgcgatcagcaaacattctagtggggaatggactcatatgcaagattgctgacttcggattggcccgattgatagaagacaatgagtacacagcaagacaaggtgcaaagttccccatcaagtggacggcccccgaggcagccctgtacgggaggttcacaatcaagtctgacgtgtggtcttttggaatcttactcacagagctggtcaccaaaggaagagtgccatacccaggcatgaacaaccgggaggtgctggagcaggtggagcgaggctacaggatgccctgcccgcaggactgccccatctctctgcatgagctcatgatccactgctggaaaaaggaccctgaagaacgccccacttttgagtacttgcagagcttcctggaagactactttaccgcgacagagccccagtaccaacctggtgaaaacctgtaaggcccgggtctgcggagagaggccttgtcccagaggctgccccacccctccccattagctttcaattccgtagccagctgctccccagcagcggaaccgcccaggatcagattgcatgtgactctgaagctgacgaacttccatggccctcattaatgacacttgtccccaaatccgaacctcctctgtgaagcattcgagacagaaccttgttatttctcagactttggaaaatgcattgtatcgatgttatgtaaaaggccaaacctctgttcagtgtaaatagttactccagtgccaacaatcctagtgctttccttttttaaaaatgcaaatcctatgtgattttaactctgtcttcacctgattcaactaaaaaaaaaaaagtattattttccaaaagtggcctctttgtctaaaacaataaaattttttttcatgttttaacaaaaaccaa

SEQ ID NO: 114 - protein, 537 aa, homo sapiens
MGCVQCKDKEATKLTEERDGSLNQSSGYRYGTDPTPQHYPSFGVTSIPNYNNFHAAGGQGLTVFGGVNSSSHTGTLRTRGGTGVTLFVALYDYEARTEDDLSFHKGEKFQILNSSEGDWWEARSLTTGETGYIPSNYVAPVDSIQAEEWYFGKLGRKDAERQLLSFGNPRGTFLIRESETTKGAYSLSIRDWDDMKGDHVKHYKIRKLDNGGYYITTRAQFETLQQLVQHYSERAAGLCCRLVVPCHKGMPRLTDLSVKTKDVWEIPRESLQLIKRLGNGQFGEVWMGTWNGNTKVAIKTLKPGTMSPESFLEEAQIMKKLKHDKLVQLYAVVSEEPIYIVTEYMNKGSLLDFLKDGEGRALKLPNLVDMAAQVAAGMAYIERMNYIHRDLRSANILVGNGLICKIADFGLARLIEDNEYTARQGAKFPIKWTAPEAALYGRFTIKSDVWSFGILLTELVTKGRVPYPGMNNREVLEQVERGYRMPCPQDCPISLHELMIHCWKKDPEERPTFEYLQSFLEDYFTATEPQYQPGENL

SEQ ID NO: 115 - DNA, 2357 bp, homo sapiens
gagccgggctactctgagaagaagacaccaagtggattctgcttcccctgggacagcactgagcgagtgtggagagaggtacagccctcggcctacaagctctttagtcttgaaagcgccacaagcagcagctgctgagccatggctgaaggggaaatcaccaccttcacagccctgaccgagaagtttaatctgcctccagggaattacaagaagcccaaactcctctactgtagcaacgggggccacttcctgaggatccttccggatggcacagtggatgggacaagggacaggagcgaccagcacattcagctgcagctcagtgcggaaagcgtgggggaggtgtatataaagagtaccgagactggccagtacttggccatggacaccgacgggcttttatacggctcacagacaccaaatgaggaatgtttgttcctggaaaggctggaggagaaccattacaacacctatatatccaagaagcatgcagagaagaattggtttgttggcctcaagaagaatgggagctgcaaacgcggtcctcggactcactatggccagaaagcaatcttgtttctccccctgccagtctcttctgattaaagagatctgttctgggtgttgaccactccagagaagtttcgaggggtcctcacctggttgacccaaaaatgttcccttgaccattggctgcgctaacccccagcccacagagcctgaatttgtaagcaacttgcttctaaatgcccagttcacttctttgcagagccttttacccctgcacagtttagaacagagggaccaaattgcttctaggagtcaactggctggccagtctgggtctgggtttggatctccaattgcctcttgcaggctgagtccctccatgcaaaagtggggctaaatgaagtgtgttaaggggtcggctaagtgggacattagtaactgcacactatttccctctactgagtaaaccctatctgtgattcccccaaacatctggcatggctcccttttgtccttcctgtgccctgcaaatattagcaaagaagcttcatgccaggttaggaaggcagcattccatgaccagaaacagggacaaagaaatccccccttcagaacagaggcatttaaaatggaaaagagagattggattttggtgggtaacttagaaggatggcatctccatgtagaataaatgaagaaagggaggcccagccgcaggaaggcagaataaatccttgggagtcattaccacgccttgaccttcccaaggttactcagcagcagagagccctgggtgacttcaggtggagagcactagaagtggtttcctgataacaagcaaggatatcagagctgggaaattcatgtggatctggggactgagtgtgggagtgcagagaaagaaagggaaactggctgaggggataccataaaaagaggatgatttcagaaggagaaggaaaaagaaagtaatgccacacattgtgcttggcccctggtaagcagaggctttggggtcctagcccagtgcttctccaacactgaagtgcttgcagatcatctggggacctggtttgaatggagattctgattcagtgggttgggggcagagtttctgcagttccatcaggtcccccccaggtgcaggtgctgacaatactgctgccttacccgccatacattaaggagcagggtcctggtcctaaagagttattcaaatgaaggtggttcgacgccccgaacctcacctgacctcaactaacccttaaaaatgcacacctcatgagtctacctgagcattcaggcagcactgacaatagttatgcctgtactaaggagcatgattttaagaggctttggccaatgcctataaaatgcccatttcgaagatatacaaaaacatacttcaaaaatgttaaacccttaccaacagcttttcccaggagaccatttgtattaccattacttgtataaatacacttcctgcttaaacttgacccaggtggctagcaaattagaaacaccattcatctctaacatatgatactgatgccatgtaaaggcctttaataagtcattgaaatttactgtgagactgtatgttttaattgcatttaaaaatatatagcttgaaagcagttaaactgattagtattcaggcactgagaatgatagtaataggatacaatgtataagctactcacttatctgatacttatttacctataaaatgagatttttgttttccactgtgctattacaaattttcttttgaaagtaggaactcttaagcaatggtaattgtgaataaaaattgatgagagtgttaaaaaaaaaaaaaaaaa

SEQ ID NO: 116 - protein, 155 aa, homo sapiens
MAEGEITTFTALTEKFNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 117 - DNA, 1029 bp, homo sapiens
tctgcttttaataagcttcccaatcagctctcgagtgcaaagcgctctccctccctcgcccagccttcgtcctcctggcccgctcctctcatccctcccattctccatttcccttccgttccctccctgtcagggcgtaattgagtcaaaggcaggatcaggttccccgccttccagtccaaaaatcccgccaagagagccccagagcagaggaaaatccaaagtggagagaggggaagaaagagaccagtgagtcatccgtccagaaggcggggagagcagcagcggcccaagcaggagctgcagcgagccgggtacctggactcagcggtagcaacctcgccccttgcaacaaaggcagactgagcgccagagaggacgtttccaactcaaaaatgcaggctcaacagtaccagcagcagcgtcgaaaatttgcagctgccttcttggcattcattttcatactggcagctgtggatactgctgaagcagggaagaaagagaaaccagaaaaaaaagtgaagaagtctgactgtggagaatggcagtggagtgtgtgtgtgcccaccagtggagactgtgggctgggcacacgggagggcactcggactggagctgagtgcaagcaaaccatgaagacccagagatgtaagatcccctgcaactggaagaagcaatttggcgcggagtgcaaataccagttccaggcctggggagaatgtgacctgaacacagccctgaagaccagaactggaagtctgaagcgagccctgcacaatgccgaatgccagaagactgtcaccatctccaagccctgtggcaaactgaccaagcccaaacctcaagcagaatctaagaagaagaaaaaggaaggcaagaaacaggagaagatgctggattaaaagatgtcacctgtggaacataaaaaggacatcagcaaacaggatcagttaactattgcatttatatgtaccgtaggctttgtattcaaaaattatctatagctaagtacacaataagcaaaaacaa

SEQ ID NO: 118 - protein, 168 aa, homo sapiens
MQAQQYQQQRRKFAAAFLAFIFILAAVDTAEAGKKEKPEKKVKKSDCGEWQWSVCVPTSGDCGLGTREGTRTGAECKQTMKTQRCKIPCNWKKQFGAECKYQFQAWGECDLNTALKTRTGSLKRALHNAECQKTVTISKPCGKLTKPKPQAESKKKKKEGKKQEKMLD

SEQ ID NO: 119 - DNA, 1350 bp, homo sapiens
aggttaatcttaaaagcccaggttacccgcggaaatttatgctgtccggtcaccgtgacaatgcagctgaggaacccagaactacatctgggctgcgcgcttgcgcttcgcttcctggccctcgtttcctgggacatccctggggctagagcactggacaatggattggcaaggacgcctaccatgggctggctgcactgggagcgcttcatgtgcaaccttgactgccaggaagagccagattcctgcatcagtgagaagctcttcatggagatggcagagctcatggtctcagaaggctggaaggatgcaggttatgagtacctctgcattgatgactgttggatggctccccaaagagattcagaaggcagacttcaggcagaccctcagcgctttcctcatgggattcgccagctagctaattatgttcacagcaaaggactgaagctagggatttatgcagatgttggaaataaaacctgcgcaggcttccctgggagttttggatactacgacattgatgcccagacctttgctgactggggagtagatctgctaaaatttgatggttgttactgtgacagtttggaaaatttggcagatggttataagcacatgtccttggccctgaataggactggcagaagcattgtgtactcctgtgagtggcctctttatatgtggccctttcaaaagcccaattatacagaaatccgacagtactgcaatcactggcgaaattttgctgacattgatgattcctggaaaagtataaagagtatcttggactggacatcttttaaccaggagagaattgttgatgttgctggaccagggggttggaatgacccagatatgttagtgattggcaactttggcctcagctggaatcagcaagtaactcagatggccctctgggctatcatggctgctcctttattcatgtctaatgacctccgacacatcagccctcaagccaaagctctccttcaggataaggacgtaattgccatcaatcaggaccccttgggcaagcaagggtaccagcttagacagggagacaactttgaagtgtgggaacgacctctctcaggcttagcctgggctgtagctatgataaaccggcaggagattggtggacctcgctcttataccatcgcagttgcttccctgggtaaaggagtggcctgtaatcctgcctgcttcatcacacagctcctccctgtgaaaaggaagctagggttctatgaatggacttcaaggttaagaagtcacataaatcccacaggcactgttttgcttcagctagaaaatacaatgcagatgtcattaaaagacttactttaa

SEQ ID NO: 120 - protein, 429 aa, homo sapiens
MQLRNPELHLGCALALRFLALVSWDIPGARALDNGLARTPTMGWLHWERFMCNLDCQEEPDSCISEKLFMEMAELMVSEGWKDAGYEYLCIDDCWMAPQRDSEGRLQADPQRFPHGIRQLANYVHSKGLKLGIYADVGNKTCAGFPGSFGYYDIDAQTFADWGVDLLKFDGCYCDSLENLADGYKHMSLALNRTGRSIVYSCEWPLYMWPFQKPNYTEIRQYCNHWRNFADIDDSWKSIKSILDWTSFNQERIVDVAGPGGWNDPDMLVIGNFGLSWNQQVTQMALWAIMAAPLFMSNDLRHISPQAKALLQDKDVIAINQDPLGKQGYQLRQGDNFEVWERPLSGLAWAVAMINRQEIGGPRSYTIAVASLGKGVACNPACFITQLLPVKRKLGFYEWTSRLRSHINPTGTVLLQLENTMQMSLKDLL

SEQ ID NO: 121 - DNA, 287 bp, homo sapiens
gtctgatttttatggttgacagtaatgacagagagcagattgatgaggcctgggaagtgctaacttacttgttagaggacgatgagctcagaaatgcagttttattggtatttgccaataaacaagatctccctaatactatgaacgcggcagagataacggacaagctcggcctccattccctccgctacagaaactggcacattcaggctacttgtgccactactggacatgggctttacgaaggcctgaactggctcgccaaccagttccagaaccagaactga

SEQ ID NO: 122 - protein, 91 aa, homo sapiens
MVDSNDREQIDEAWEVLTYLLEDDELRNAVLLVFANKQDLPNTMNAAEITDKLGLHSLRYRNWHIQATCATTGHGLYEGLNWLANQFQNQN

SEQ ID NO: 123 - DNA, 3781 bp, homo sapiens
ctgtctgagcatttcactgcggagcctgagcgcgcctgcctgggaaaacactgcagcggtgctcggactcctcctgtccagcaggaggcgcggcccggcagctcccgcatgcgcagtgcgctcggtgtcagacggcccggatcccggttaccggcccctcgctcgctgctcgccagcccagactcggccctggcagtggcggctggcgattcggaccgatccgacctgggcggaggtggcccgcgccccgcggcatgagccggtgaccaagctcggggccgagcgggaggcagccgtggccgagccacagggatctgattcctcccagggggatgtcctgcgcctcagggtccggtggtggcctgcggcatcccttgcggtgccagaagccgtgggacgagtgtctttaatgttatagagctactcagagttgctgtttctccttgagatgcttttggagtgtgaggaagaggctgtctgtgtcattatgtgtgcgtcggtcaagtataatatccggggtcctgccctcatcccaagaatgaagaccaagcaccgaatctactatatcaccctcttctccattgtcctcctgggcctcattgccactggcatgtttcagttttggccccattctatcgagtcctcaaatgactggaatgtagagaagcgcagcatccgtgatgtgccggttgttaggctgccagccgacagtcccatcccagagcggggggatctcagttgcagaatgcacacgtgttttgatgtctatcgctgtggcttcaacccaaagaacaaaatcaaggtgtatatctatgctctgaaaaagtacgtggatgactttggcgtctctgtcagcaacaccatctcccgggagtataatgaactgctcatggccatctcagacagtgactactacactgatgacatcaaccgggcctgtctgtttgttccctccatcgatgtgcttaaccagaacacactgcgcatcaaggagacagcacaagcgatggcccagctctctaggtgggatcgaggtacgaatcacctgttgttcaacatgttgcctggaggtcccccagattataacacagccctggatgtccccagagacagggccctgttggctggtggcggcttttctacgtggacttaccggcaaggctacgatgtcagcattcctgtctatagtccactgtcagctgaggtggatcttccagagaaaggaccaggtccacggcaatacttcctcctgtcatctcaggtgggtctccatcctgagtacagagaggacctagaagccctccaggtcaaacatggagagtcagtgttagtactcgataaatgcaccaacctctcagagggtgtcctttctgtccgtaagcgctgccacaagcaccaggtcttcgattacccacaggtgctacaggaggctactttctgtgtggttcttcgtggagctcggctgggccaggcagtattgagcgatgtgttacaagctggctgtgtcccggttgtcattgcagactcctatattttgcctttctctgaagttcttgactggaagagagcatctgtggttgtaccagaagaaaagatgtcagatgtgtacagtattttgcagagcatcccccaaagacagattgaagaaatgcagagacaggcccggtggttctgggaagcgtacttccagtcaattaaagccattgccctggccaccctgcagattatcaatgaccggatctatccatatgctgccatctcctatgaagaatggaatgaccctcctgctgtgaagtggggcagcgtgagcaatccactcttcctcccgctgatcccaccacagtctcaagggttcaccgccatagtcctcacctacgaccgagtagagagcctcttccgggtcatcactgaagtgtccaaggtgcccagtctatccaaactacttgtcgtctggaataatcagaataaaaaccctccagaagattctctctggcccaaaatccgggttccattaaaagttgtgaggactgctgaaaacaagttaagtaaccgtttcttcccttatgatgaaatcgagacagaagctgttctggccattgatgatgatatcattatgctgacctctgacgagctgcaatttggttatgaggtctggcgggaatttcctgaccggttggtgggttacccgggtcgtctgcatctctgggaccatgagatgaataagtggaagtatgagtctgagtggacgaatgaagtgtccatggtgctcactggggcagctttttatcacaagtattttaattacctgtatacctacaaaatgcctggggatatcaagaactgggtagatgctcatatgaactgtgaagatattgccatgaacttcctggtggccaacgtcacgggaaaagcagttatcaaggtaaccccacgaaagaaattcaagtgtcctgagtgcacagccatagatgggctttcactagaccaaacacacatggtggagaggtcagagtgcatcaacaagtttgcttcagtcttcgggaccatgcctctcaaggtggtggaacaccgagctgaccctgtcctgtacaaagatgactttcctgagaagctgaagagcttccccaacattggcagcttatgaaacgtgtcattggtggaggtctgaatgtgaggctgggacagagggagagaacaaggcctcccagcactctgatgtcagagtagtaggttaagggtggaaggttgacctacttggatcttggcatgcacccacctaacccactttctcaagaacaagaacctagaatgaatatccaagcacctcgagctatgcaacctctgttcttgtatttcttatgatctctgatgggttcttctcgaaaatgccaagtggaagactttgtggcatgctccagatttaaatccagctgaggctccctttgttttcagttccatgtaacaatctggaaggaaacttcacggacaggaagactgctggagaagagaagcgtgttagcccatttgaggtctggggaatcatgtaaagggtacccagacctcacttttagttatttacatcaatgagttctttcagggaaccaaacccagaattcggtgcaaaagccaaacatcttggtgggatttgataaatgccttgggacctggagtgctgggcttgtgcacaggaagagcaccagccgctgagtcaggatcctgtcagttccatgagctattcctctttggtttggctttttgatatgattaaaattattttttattcctttttctactgtgtcttaaacaccaattcctgatagtccaaggaaccacctttctcccttgatatatttaactccgtctttggcctgacaacagtcttctgcccatgtctgggaacacacgccaggaggaatgtctgataccctctgcatcaagcgtaagaaggtcccaaatcataaccattttaagaacagatgactcagaaacctccagaggaatctgtttgcttcctgattagatccagtcaatgttttaaaggtattgtcagagaaaaacagagggtctgtactagccatgcaaggagtcgctctagctggtacccgtaaaagttgtgggattgtgaccccccatcccaaggggatgccaaaatttctctcattcttttggtataaacttaacattagccagggaggttctggctaacgttaaatgctgctatacaactgctttgcaacagttgctggtatatttaaatcattaaatttcagcatttactaat

SEQ ID NO: 124 - protein, 718 aa, homo sapiens
MCASVKYNIRGPALIPRMKTKHRIYYITLFSIVLLGLIATGMFQFWPHSIESSNDWNVEKRSIRDVPVVRLPADSPIPERGDLSCRMHTCFDVYRCGFNPKNKIKVYIYALKKYVDDFGVSVSNTISREYNELLMAISDSDYYTDDINRACLFVPSIDVLNQNTLRIKETAQAMAQLSRWDRGTNHLLFNMLPGGPPDYNTALDVPRDRALLAGGGFSTWTYRQGYDVSIPVYSPLSAEVDLPEKGPGPRQYFLLSSQVGLHPEYREDLEALQVKHGESVLVLDKCTNLSEGVLSVRKRCHKHQVFDYPQVLQEATFCVVLRGARLGQAVLSDVLQAGCVPVVIADSYILPFSEVLDWKRASVVVPEEKMSDVYSILQSIPQRQIEEMQRQARWFWEAYFQSIKAIALATLQIINDRIYPYAAISYEEWNDPPAVKWGSVSNPLFLPLIPPQSQGFTAIVLTYDRVESLFRVITEVSKVPSLSKLLVVWNNQNKNPPEDSLWPKIRVPLKVVRTAENKLSNRFFPYDEIETEAVLAIDDDIIMLTSDELQFGYEVWREFPDRLVGYPGRLHLWDHEMNKWKYESEWTNEVSMVLTGAAFYHKYFNYLYTYKMPGDIKNWVDAHMNCEDIAMNFLVANVTGKAVIKVTPRKKFKCPECTAIDGLSLDQTHMVERSECINKFASVFGTMPLKVVEHRADPVLYKDDFPEKLKSFPNIGSL

SEQ ID NO: 125 - DNA, 1908 bp, homo sapiens
ctctgtctcattccctcgcgctctctcgggcaacatggcgggtgtggaggaggtagcggcctccgggagccacctgaatggcgacctggatccagacgacagggaagaaggagctgcctctacggctgaggaagcagccaagaaaaaaagacgaaagaagaagaagagcaaagggccttctgcagcaggggaacaggaacctgataaagaatcaggagcctcagtggatgaagtagcaagacagttggaaagatcagcattggaagataaagaaagagatgaagatgatgaagatggagatggcgatggagatggagcaactggaaagaagaagaaaaagaagaagaagaagagaggaccaaaagttcaaacagaccctccctcagttccaatatgtgacctgtatcctaatggtgtatttcccaaaggacaagaatgcgaatacccacccacacaagatgggcgaacagctgcttggagaactacaagtgaagaaaagaaagcattagatcaggcaagtgaagagatttggaatgattttcgagaagctgcagaagcacatcgacaagttagaaaatacgtaatgagctggatcaagcctgggatgacaatgatagaaatctgtgaaaagttggaagactgttcacgcaagttaataaaagagaatggattaaatgcaggcctggcatttcctactggatgttctctcaataattgtgctgcccattatactcccaatgccggtgacacaacagtattacagtatgatgacatctgtaaaatagactttggaacacatataagtggtaggattattgactgtgcttttactgtcacttttaatcccaaatatgatacgttattaaaagctgtaaaagatgctactaacactggaataaagtgtgctggaattgatgttcgtctgtgtgatgttggtgaggccatccaagaagttatggagtcctatgaagttgaaatagatgggaagacatatcaagtgaaaccaatccgtaatctaaatggacattcaattgggcaatatagaatacatgctggaaaaacagtgccgattgtgaaaggaggggaggcaacaagaatggaggaaggagaagtatatgcaattgaaacctttggtagtacaggaaaaggtgttgttcatgatgatatggaatgttcacattacatgaaaaattttgatgttggacatgtgccaataaggcttccaagaacaaaacacttgttaaatgtcatcaatgaaaactttggaacccttgccttctgccgcagatggctggatcgcttgggagaaagtaaatacttgatggctctgaagaatctgtgtgacttgggcattgtagatccatatccaccattatgtgacattaaaggatcatatacagcgcaatttgaacataccatcctgttgcgtccaacatgtaaagaagttgtcagcagaggagatgactattaaacttagtccaaagccacctcaacacctttattttctgagctttgttggaaaacatgataccagaattaatttgccacatgttgtctgttttaacagtggacccatgtaatacttttatccatgtttaaaaagaaggaatttggacaaaggcaaaccgtctaatgtaattaaccaacgaaaaagctttccggacttttaaatgctaactgtttttccccttcctgtctaggaaaatgctataaagctcaaattagttaggaatgacttatacgttttgttttgaatacctaagagatactttttggatatttatattgccatattcttacttgaatgctttgaatgactacatccagttctgcacctataccctctggtgttgctttttaaccttcctggaatccatttctaaaaaataaagacattttcagatctga

SEQ ID NO: 126 - protein, 478 aa, homo sapiens
MAGVEEVAASGSHLNGDLDPDDREEGAASTAEEAAKKKRRKKKKSKGPSAAGEQEPDKESGASVDEVARQLERSALEDKERDEDDEDGDGDGDGATGKKKKKKKKKRGPKVQTDPPSVPICDLYPNGVFPKGQECEYPPTQDGRTAAWRTTSEEKKALDQASEEIWNDFREAAEAHRQVRKYVMSWIKPGMTMIEICEKLEDCSRKLIKENGLNAGLAFPTGCSLNNCAAHYTPNAGDTTVLQYDDICKIDFGTHISGRIIDCAFTVTFNPKYDTLLKAVKDATNTGIKCAGIDVRLCDVGEAIQEVMESYEVEIDGKTYQVKPIRNLNGHSIGQYRIHAGKTVPIVKGGEATRMEEGEVYAIETFGSTGKGVVHDDMECSHYMKNFDVGHVPIRLPRTKHLLNVINENFGTLAFCRRWLDRLGESKYLMALKNLCDLGIVDPYPPLCDIKGSYTAQFEHTILLRPTCKEVVSRGDDY

SEQ ID NO: 127 - DNA, 12689 bp, homo sapiens
aaaattccttagttgctggctttgaccttttatgttgctgagttttacacatctattttctcaactgccatatcctagggggcttggagtacccataatacagtgagcccaccttcctggtccccagacatttcagaaggtcgggaaatttttaaacccaggcagcttcctggcagtgccatttggagcatcaaagtgggccgtgggtctggatttccaggaaagcggagacctcgaggtgcaggactgtcggggcgaggtggccgaggcaggtcaaagctgaaaagtggaatcggagctgttgtattacctggggtgtctactgcagatatttcatcaaataaggatgatgaagaaaactctatgcacaatacagttgtgttgttttctagcagtgacaagttcactttgaatcaggatatgtgtgtagtttgtggcagttttggccaaggagcagaaggaagattacttgcctgttctcagtgtggtcagtgttaccatccatactgtgtcagtattaagatcactaaagtggttcttagcaaaggttggaggtgtcttgagtgcactgtgtgtgaggcctgtgggaaggcaactgacccaggaagactcctgctgtgtgatgactgtgacataagttatcacacctactgcctagaccctccattgcagacagttcccaaaggaggctggaagtgcaaatggtgtgtttggtgcagacactgtggagcaacatctgcaggtctaagatgtgaatggcagaacaattacacacagtgcgctccttgtgcaagcttatcttcctgtccagtctgctatcgaaactatagagaagaagatcttattctgcaatgtagacaatgtgatagatggatgcatgcagtttgtcagaacttaaatactgaggaagaagtggaaaatgtagcagacattggttttgattgtagcatgtgcagaccctatatgcctgcgtctaatgtgccttcctcagactgctgtgaatcttcacttgtagcacaaattgtcacaaaagtaaaagagctagacccacccaagacttatacccaggatggtgtgtgtttgactgaatcagggatgactcagttacagagcctcacagttacagttccaagaagaaaacggtcaaaaccaaaattgaaattgaagattataaatcagaatagcgtggccgtccttcagacccctccagacatccaatcagagcattcaagggatggtgaaatggatgatagtcgagaaggagaacttatggattgtgatggaaaatcagaatctagtcctgagcgggaagctgtggatgatgaaactaagggagtggaaggaacagatggtgtcaaaaagagaaaaaggaaaccatacagaccaggtattggtggatttatggtgcggcaaagaagtcgaactgggcaagggaaaaccaaaagatctgtgatcagaaaagattcctcaggctctatttccgagcagttaccttgcagagatgatggctggagtgagcagttaccagatactttagttgatgaatctgtttctgttactgaaagcactgaaaaaataaagaagagataccgaaaaaggaaaaataagcttgaagaaactttccctgcctatttacaagaagctttctttggaaaagatcttctagatacaagtagacaaagcaagataagtttagataatctgtcagaagatggagctcagcttttatataaaacaaacatgaacacaggtttcttggatccttccttagatccactacttagttcatcctcggctccaacaaaatctggaactcacggtcctgctgatgacccattagctgatatttctgaagttttaaacacagatgatgacattcttggaataatttcagatgatctagcaaaatcagttgatcattcagatattggtcctgtcactgatgatccttcctctttgcctcagccaaatgtcaatcagagttcacgaccattaagtgaagaacagctagatgggatcctcagtcctgaactagacaaaatggtcacagatggagcaattcttggaaaattatataaaattccagagcttggcggaaaagatgttgaagacttatttacagctgtacttagtcctgcgaacactcagccaactccattgccacagcctcccccaccaacacagctgttgccaatacacaatcaggatgctttttcacggatgcctctcatgaatggccttattggatccagtcctcatctcccacataattctttgccacctggaagcggactgggaactttctctgcaattgcacaatcctcttatcctgatgccagggataaaaattcagcctttaatccaatggcaagtgatcctaacaactcttggacatcatcagctcccactgtggaaggagaaaatgacacaatgtcgaatgcccagagaagcacgcttaagtgggagaaagaggaggctctgggtgaaatggcaactgttgccccagttctctacaccaatattaatttccccaacttaaaggaagaattccctgattggactactagagtgaagcaaattgccaaattgtggagaaaagcaagctcacaagaaagagcaccatatgtgcaaaaagccagagataacagagctgctttacgcattaataaagtacagatgtcaaatgattccatgaaaaggcagcaacagcaagatagcattgatcccagctctcgtattgattcggagctttttaaagatcctttaaagcaaagagaatcagaacatgaacaggaatggaaatttagacagcaaatgcgtcagaaaagtaagcagcaagctaaaattgaagccacacagaaacttgaacaggtgaaaaatgagcagcagcagcagcaacaacagcaatttggttctcagcatcttctggtgcagtctggttcagatacaccaagtagtgggatacagagtcccttgacacctcagcctggcaatggaaatatgtctcctgcacagtcattccataaagaactgtttacaaaacagccacccagtacccctacgtctacatcttcagatgatgtgtttgtaaagccacaagctccacctcctcctccagccccatcccggattcccatccaggatagtctttctcaggctcagacttctcagccaccctcaccgcaagtgttttcacctgggtcctctaactcacgaccaccatctccaatggatccatatgcaaaaatggttggtacccctcgaccacctcctgtgggccatagtttttccagaagaaattctgctgcaccagtggaaaactgtacacctttatcatcggtatctaggccccttcaaatgaatgagacaacagcaaataggccatcccctgtcagagatttatgttcttcttccacgacaaataatgacccctatgcaaaacctccagacacacctaggcctgtgatgacagatcaatttcccaaatccttgggcctatcccggtctcctgtagtttcagaacaaactgcaaaaggccctatagcagctggaaccagtgatcactttactaaaccatctcctagggcagatgtgtttcaaagacaaaggatacctgactcatatgcacgacccttgttgacacctgcacctcttgatagtggtcctggaccttttaagactccaatgcaacctcctccatcctctcaggatccttatggatcagtgtcacaggcatcaaggcgattgtctgttgacccttatgaaaggcctgctttgacaccaagacctatagataatttttctcataatcagtcaaatgatccatatagtcagcctccccttaccccacatccagcagtgaatgaatcttttgcccatccttcaagggctttttcccagcctggaaccatatcaaggccaacatctcaggacccatactcccaacccccaggaactccacgacctgttgtagattcttattcccaatcttcaggaacagctaggtccaatacagacccttactctcaacctcctggaactccccggcctactactgttgacccatatagtcagcagccccaaaccccaagaccatctacacaaactgacttgtttgttacacctgtaacaaatcagaggcattctgatccatatgctcatcctcctggaacaccaagacctggaatttctgtcccttactctcagccaccagcaacaccaaggccaaggatttcagagggttttactaggtcctcaatgacaagaccagtcctcatgccaaatcaggatcctttcctgcaagcagcacaaaaccgaggaccagctttacctggcccgttggtaaggccacctgatacatgttcccagacacctaggccccctggacctggtctttcagacacatttagccgtgtttccccatctgctgcccgtgatccctatgatcagtctccaatgactccaagatctcagtctgactcttttggaacaagtcaaactgcccatgatgttgctgatcagccaaggcctggatcagaggggagcttctgtgcatcttcaaactctccaatgcactcccaaggccagcagttctctggtgtctcccaacttcctggacctgtgccaacttcaggagtaactgatacacagaatactgtaaatatggcccaagcagatacagagaaattgagacagcggcagaagttacgtgaaatcattctccagcagcaacagcagaagaagattgcaggtcgacaggagaaggggtcacaggactcacccgcagtgcctcatccagggcctcttcaacactggcaaccagagaatgttaaccaggctttcaccagacccccacctccctatcctgggaacattaggtctcctgttgcccctcctttaggacctagatatgctgttttcccaaaagatcagcgtggaccctatcctcctgatgttgctagtatggggatgagacctcatggatttagatttggatttccaggaggtagtcatggtaccatgccgagtcaagagcgcttccttgtgcctcctcagcaaatacagggatctggagtttctccacagctaagaagatcagtatctgtagatatgcctaggcctttaaataactcacaaatgaataatccagttggacttcctcagcatttttcaccacagagcttgccagttcagcagcacaacatactgggccaagcatatattgaactgagacatagggctcctgacggaaggcaacggctgcctttcagtgctccacctggcagcgttgtagaggcatcttctaatctgagacatggaaacttcattccccggccagactttccgggccctagacacacagaccccatgcgacgacctccccagggtctacctaatcagctacctgtgcacccagatttggaacaagtgccaccatctcaacaagagcaaggtcattctgtccattcatcttctatggtcatgaggactctgaaccatccactaggtggtgaattttcagaagctcctttgtcaacatctgtaccgtctgaaacaacgtctgataatttacagataaccacccagccttctgatggtctagaggaaaaacttgattctgatgacccttctgtgaaggaactggatgttaaagaccttgagggggttgaagtcaaagacttagatgatgaagatcttgaaaacttaaatttagatacagaggatggcaaggtagttgaattggatactttagataatttggaaactaatgatcccaacctggatgacctcttaaggtcaggagagtttgatatcattgcatatacagatccagaacttgacatgggagataagaaaagcatgtttaatgaggaactagaccttccaattgatgataagttagataatcagtgtgtatctgttgaaccaaaaaaaaaggaacaagaaaacaaaactctggttctctctgataaacattcaccacagaaaaaatccactgttaccaatgaggtaaaaacggaagtactgtctccaaattctaaggtggaatccaaatgtgaaactgaaaaaaatgatgagaataaagataatgttgacactccttgctcacaggcttctgctcactcagacctaaatgatggagaaaagacttctttgcatccttgtgatccagatctatttgagaaaagaaccaatcgagaaactgctggccccagtgcaaatgtcattcaggcatccactcaactacctgctcaagatgtaataaactcttgtggcataactggatcaactccagttctctcaagtttacttgctaatgagaaatctgataattcagacattaggccatcggggtctccaccaccaccaactctgccggcctccccatccaatcatgtgtcaagtttgcctcctttcatagcaccgcctggccgtgttttggataatgccatgaattctaatgtgacagtagtctctagggtaaaccatgttttttctcagggtgtgcaggtaaacccagggctcattccaggtcaatcaacagttaaccacagtctggggacaggaaaacctgcaactcaaactgggcctcaaacaagtcagtctggtaccagtagcatgtctggaccccaacagctaatgattcctcaaacattagcacagcagaatagagagaggccccttcttctagaagaacagcctctacttctacaggatcttttggatcaagaaaggcaagaacagcagcagcaaagacagatgcaagccatgattcgtcagcgatcagaaccgttcttccctaatattgattttgatgcaattacagatcctataatgaaagccaaaatggtggcccttaaaggtataaataaagtgatggcacaaaacaatctgggcatgccaccaatggtgatgagcaggttcccttttatgggccaggtggtaactggaacacagaacagtgaaggacagaaccttggaccacaggccattcctcaggatggcagtataacacatcagatttctaggcctaatcctccaaattttggtccaggctttgtcaatgattcacagcgtaagcagtatgaagagtggctccaggagacccaacagctgcttcaaatgcagcagaagtatcttgaagaacaaattggtgctcacagaaaatctaagaaggccctttcagctaaacaacgtactgccaagaaagctgggcgtgaatttccagaggaagatgcagaacaactcaagcatgttactgaacagcaaagcatggttcagaaacagctagaacagattcgtaaacaacagaaagaacatgctgaattgattgaagattatcggatcaaacagcagcagcaatgtgcaatggccccacctaccatgatgcccagtgtccagccccagccacccctaattccaggtgccactccacccaccatgagccaacccacctttcccatggtgccacagcagcttcagcaccagcagcacacaacagttatttctggccatactagccctgttagaatgcccagtttacctggatggcaacccaacagtgctcctgcccacctgcccctcaatcctcctagaattcagcccccaattgcccagttaccaataaaaacttgtacaccagccccagggacagtctcaaatgcaaatccacagagtggaccaccacctcgggtagaatttgatgacaacaatccctttagtgaaagttttcaagaacgggaacgtaaggaacgtttacgagaacagcaagagagacaacggatccaactcatgcaggaggtagatagacaaagagctttgcagcagaggatggaaatggagcagcatggtatggtgggctctgagataagtagtagtaggacatctgtgtcccagattcccttctacagttccgacttaccttgtgattttatgcaacctctaggaccccttcagcagtctccacaacaccaacagcaaatggggcaggttttacagcagcagaatatacaacaaggatcaattaattcaccctccacccaaactttcatgcagactaatgagcgaaggcaggtaggccctccttcatttgttcctgattcaccatcaatccctgttggaagcccaaatttttcttctgtgaagcagggacatggaaatctttctgggaccagcttccagcagtccccagtgaggccttcttttacacctgctttaccagcagcacctccagtagctaatagcagtctcccatgtggccaagattctactataacccatggacacagttatccgggatcaacccaatcgctcattcagttgtattctgatataatcccagaggaaaaagggaaaaagaaaagaacaagaaagaagaaaagagatgatgatgcagaatccaccaaggctccatcaactccccattcagatataactgccccaccgactccaggcatctcagaaactacctctactcctgcagtgagcacacccagtgagcttcctcaacaagccgaccaagagtcggtggaaccagtcggcccatccactcccaatatggcagcaggccagctatgtacagaattagagaacaaactgcccaatagtgatttctcacaagcaactccaaatcaacagacgtatgcaaattcagaagtagacaagctctccatggaaacccctgccaaaacagaagagataaaactggaaaaggctgagacagagtcctgcccaggccaagaggagcctaaattggaggaacagaatggtagtaaggtagaaggaaacgctgtagcctgtcctgtctcctcagcacagagtcctccccattctgctggggcccctgctgccaaaggagactcagggaatgaacttctgaaacacttgttgaaaaataaaaagtcatcttctcttttgaatcaaaaacctgagggcagtatttgttcagaagatgactgtacaaaggataataaactagttgagaagcagaacccagctgaaggactgcaaactttgggggctcaaatgcaaggtggttttggatgtggcaaccagttgccaaaaacagatggaggaagtgaaaccaagaaacagcgaagcaaacggactcagaggacgggtgagaaagcagcacctcgctcaaagaaaaggaaaaaggacgaagaggagaaacaagctatgtactctagcactgacacgtttacccacttgaaacaggtgaggcagctctctctgctccctctaatggaaccaatcattggagtgaactttgcgcactttcttccttatggcagtggccaatttaatagtgggaatcgacttctaggaacttttggcagtgctaccctggaaggggtttcggactactattctcagttgatctacaagcagaataatttaagtaatcctccaacaccccctgcctctcttcctcctacaccacctcctatggcttgtcagaagatggccaatggttttgcaacaactgaagaacttgctggaaaagccggagtgttagtgagccatgaagttaccaaaactctaggacctaaaccatttcagctgcccttcagaccccaggacgacttgttggcccgagctcttgctcagggccccaagacagttgatgtgccagcctccctcccaacaccacctcataacaatcaggaagaattaaggatacaggatcactgtggtgatcgagatactcctgacagttttgttccctcatcctctcctgagagtgtggttggggtagaagtgagcaggtatccagatctgtcattggtcaaggaggagcctccagaaccggtgccgtcccccatcattccaattcttcctagcactgctgggaaaagttcagaatcaagaaggaatgacatcaaaactgagccaggcactttatattttgcgtcaccttttggtccttccccaaatggtcccagatcaggtcttatatctgtagcaattactctgcatcctacagctgctgagaacattagcagtgttgtggctgcattttccgaccttcttcacgtccgaatccctaacagctatgaggttagcagtgctccagatgtcccatccatgggtttggtcagtagccacagaatcaacccgggtttggagtatcgacagcatttacttctccgtgggcctccgccaggatctgcaaaccctcccagattagtgagctcttaccggctgaagcagcctaatgtaccatttcctccaacaagcaatggtctttctggatataaggattctagtcatggtattgcagaaagcgcagcactcagaccacagtggtgttgtcattgtaaagtggttattcttggaagtggtgtgcggaaatctttcaaagatctgacccttttgaacaaggattcccgagaaagcaccaagagggtagagaaggacattgtcttctgtagtaataactgctttattctttattcatcaactgcacaagcgaaaaactcagaaaacaaggaatccattccttcattgccacaatcacctatgagagaaacgccttccaaagcatttcatcagtacagcaacaacatctccactttggatgtgcactgtctcccccagctcccagagaaagcttctccccctgcctcaccacccatcgccttccctcctgcttttgaagcagcccaagtcgaggccaagccagatgagctgaaggtgacagtcaagctgaagcctcggctaagagctgtccatggtgggtttgaagattgcaggccgctcaataaaaaatggagaggaatgaaatggaagaagtggagcattcatattgtaatccctaaggggacatttaaaccaccttgtgaggatgaaatagatgaatttctaaagaaattgggcacttcccttaaacctgatcctgtgcccaaagactatcggaaatgttgcttttgtcatgaagaaggtgatggattgacagatggaccagcaaggctactcaaccttgacttggatctgtgggtccacttgaactgcgctctgtggtccacggaggtctatgagactcaggctggtgccttaataaatgtggagctagctctgaggagaggcctacaaatgaaatgtgtcttctgtcacaagacgggtgccactagtggatgccacagatttcgatgcaccaacatttatcacttcacttgcgccattaaagcacaatgcatgttttttaaggacaaaactatgctttgccccatgcacaaaccaaagggaattcatgagcaagaattaagttactttgcagtcttcaggagggtctatgttcagcgtgatgaggtgcgacagattgctagcatcgtgcaacgaggagaacgggaccatacctttcgcgtgggtagcctcatcttccacacaattggtcagctgcttccacagcagatgcaagcattccattctcctaaagcactcttccctgtgggctatgaagccagccggctgtactggagcactcgctatgccaataggcgctgccgctacctgtgctccattgaggagaaggatgggcgcccagtgtttgtcatcaggattgtggaacaaggccatgaagacctggttctaagtgacatctcacctaaaggtgtctgggataagattttggagcctgtggcatgtgtgagaaaaaagtctgaaatgctccagcttttcccagcgtatttaaaaggagaggatctgtttggcctgaccgtctctgcagtggcacgcatagcggaatcacttcctggggttgaggcatgtgaaaattataccttccgatacggccgaaatcctctcatggaacttcctcttgccgttaaccccacaggttgtgcccgttctgaacctaaaatgagtgcccatgtcaagaggcctcacaccttaaacagcaccagcacctcaaagtcatttcagagcacagtcactggagaactgaacgcaccttatagtaaacagtttgttcactccaagtcatcgcagtaccggaagatgaaaactgaatggaaatccaatgtgtatctggcacggtctcggattcaggggctgggcctgtatgctgctcgagacattgagaaacacaccatggtcattgagtacatcgggactatcattcgaaacgaagtagccaacaggaaagagaagctttatgagtctcagaaccgtggtgtgtacatgttccgcatggataacgaccatgtgattgacgcgacgctcacaggagggcccgcaaggtatatcaaccattcgtgtgcacctaattgtgtggctgaagtggtgacttttgagagaggacacaaaattatcatcagctccagtcggagaatccagaaaggagaagagctctgctatgactataagtttgactttgaagatgaccagcacaagattccgtgtcactgtggagctgtgaactgccggaagtggatgaactgaaatgcattccttgctagctcagcgggcggcttgtccctaggaagaggcgattcaacacaccattggaattttgcagacagaaagagatttttgttttctgttttatgactttttgaaaaagcttctgggagttctgatttcctcagtcctttaggttaaagcagcgccaggaggaagctgacagaagcagcgttcctgaagtggccgaggttaaacggaatcacagaatggtccagcacttttgcttt

SEQ ID NO: 128 - protein, 4025 aa, homo sapiens
MHNTVVLFSSSDKFTLNQDMCVVCGSFGQGAEGRLLACSQCGQCYHPYCVSIKITKVVLSKGWRCLECTVCEACGKATDPGRLLLCDDCDISYHTYCLDPPLQTVPKGGWKCKWCVWCRHCGATSAGLRCEWQNNYTQCAPCASLSSCPVCYRNYREEDLILQCRQCDRWMHAVCQNLNTEEEVENVADIGFDCSMCRPYMPASNVPSSDCCESSLVAQIVTKVKELDPPKTYTQDGVCLTESGMTQLQSLTVTVPRRKRSKPKLKLKIINQNSVAVLQTPPDIQSEHSRDGEMDDSREGELMDCDGKSESSPEREAVDDETKGVEGTDGVKKRKRKPYRPGIGGFMVRQRSRTGQGKTKRSVIRKDSSGSISEQLPCRDDGWSEQLPDTLVDESVSVTESTEKIKKRYRKRKNKLEETFPAYLQEAFFGKDLLDTSRQSKISLDNLSEDGAQLLYKTNMNTGFLDPSLDPLLSSSSAPTKSGTHGPADDPLADISEVLNTDDDILGIISDDLAKSVDHSDIGPVTDDPSSLPQPNVNQSSRPLSEEQLDGILSPELDKMVTDGAILGKLYKIPELGGKDVEDLFTAVLSPANTQPTPLPQPPPPTQLLPIHNQDAFSRMPLMNGLIGSSPHLPHNSLPPGSGLGTFSAIAQSSYPDARDKNSAFNPMASDPNNSWTSSAPTVEGENDTMSNAQRSTLKWEKEEALGEMATVAPVLYTNINFPNLKEEFPDWTTRVKQIAKLWRKASSQERAPYVQKARDNRAALRINKVQMSNDSMKRQQQQDSIDPSSRIDSELFKDPLKQRESEHEQEWKFRQQMRQKSKQQAKIEATQKLEQVKNEQQQQQQQQFGSQHLLVQSGSDTPSSGIQSPLTPQPGNGNMSPAQSFHKELFTKQPPSTPTSTSSDDVFVKPQAPPPPPAPSRIPIQDSLSQAQTSQPPSPQVFSPGSSNSRPPSPMDPYAKMVGTPRPPPVGHSFSRRNSAAPVENCTPLSSVSRPLQMNETTANRPSPVRDLCSSSTTNNDPYAKPPDTPRPVMTDQFPKSLGLSRSPVVSEQTAKGPIAAGTSDHFTKPSPRADVFQRQRIPDSYARPLLTPAPLDSGPGPFKTPMQPPPSSQDPYGSVSQASRRLSVDPYERPALTPRPIDNFSHNQSNDPYSQPPLTPHPAVNESFAHPSRAFSQPGTISRPTSQDPYSQPPGTPRPVVDSYSQSSGTARSNTDPYSQPPGTPRPTTVDPYSQQPQTPRPSTQTDLFVTPVTNQRHSDPYAHPPGTPRPGISVPYSQPPATPRPRISEGFTRSSMTRPVLMPNQDPFLQAAQNRGPALPGPLVRPPDTCSQTPRPPGPGLSDTFSRVSPSAARDPYDQSPMTPRSQSDSFGTSQTAHDVADQPRPGSEGSFCASSNSPMHSQGQQFSGVSQLPGPVPTSGVTDTQNTVNMAQADTEKLRQRQKLREIILQQQQQKKIAGRQEKGSQDSPAVPHPGPLQHWQPENVNQAFTRPPPPYPGNIRSPVAPPLGPRYAVFPKDQRGPYPPDVASMGMRPHGFRFGFPGGSHGTMPSQERFLVPPQQIQGSGVSPQLRRSVSVDMPRPLNNSQMNNPVGLPQHFSPQSLPVQQHNILGQAYIELRHRAPDGRQRLPFSAPPGSVVEASSNLRHGNFIPRPDFPGPRHTDPMRRPPQGLPNQLPVHPDLEQVPPSQQEQGHSVHSSSMVMRTLNHPLGGEFSEAPLSTSVPSETTSDNLQITTQPSDGLEEKLDSDDPSVKELDVKDLEGVEVKDLDDEDLENLNLDTEDGKVVELDTLDNLETNDPNLDDLLRSGEFDIIAYTDPELDMGDKKSMFNEELDLPIDDKLDNQCVSVEPKKKEQENKTLVLSDKHSPQKKSTVTNEVKTEVLSPNSKVESKCETEKNDENKDNVDTPCSQASAHSDLNDGEKTSLHPCDPDLFEKRTNRETAGPSANVIQASTQLPAQDVINSCGITGSTPVLSSLLANEKSDNSDIRPSGSPPPPTLPASPSNHVSSLPPFIAPPGRVLDNAMNSNVTVVSRVNHVFSQGVQVNPGLIPGQSTVNHSLGTGKPATQTGPQTSQSGTSSMSGPQQLMIPQTLAQQNRERPLLLEEQPLLLQDLLDQERQEQQQQRQMQAMIRQRSEPFFPNIDFDAITDPIMKAKMVALKGINKVMAQNNLGMPPMVMSRFPFMGQVVTGTQNSEGQNLGPQAIPQDGSITHQISRPNPPNFGPGFVNDSQRKQYEEWLQETQQLLQMQQKYLEEQIGAHRKSKKALSAKQRTAKKAGREFPEEDAEQLKHVTEQQSMVQKQLEQIRKQQKEHAELIEDYRIKQQQQCAMAPPTMMPSVQPQPPLIPGATPPTMSQPTFPMVPQQLQHQQHTTVISGHTSPVRMPSLPGWQPNSAPAHLPLNPPRIQPPIAQLPIKTCTPAPGTVSNANPQSGPPPRVEFDDNNPFSESFQERERKERLREQQERQRIQLMQEVDRQRALQQRMEMEQHGMVGSEISSSRTSVSQIPFYSSDLPCDFMQPLGPLQQSPQHQQQMGQVLQQQNIQQGSINSPSTQTFMQTNERRQVGPPSFVPDSPSIPVGSPNFSSVKQGHGNLSGTSFQQSPVRPSFTPALPAAPPVANSSLPCGQDSTITHGHSYPGSTQSLIQLYSDIIPEEKGKKKRTRKKKRDDDAESTKAPSTPHSDITAPPTPGISETTSTPAVSTPSELPQQADQESVEPVGPSTPNMAAGQLCTELENKLPNSDFSQATPNQQTYANSEVDKLSMETPAKTEEIKLEKAETESCPGQEEPKLEEQNGSKVEGNAVACPVSSAQSPPHSAGAPAAKGDSGNELLKHLLKNKKSSSLLNQKPEGSICSEDDCTKDNKLVEKQNPAEGLQTLGAQMQGGFGCGNQLPKTDGGSETKKQRSKRTQRTGEKAAPRSKKRKKDEEEKQAMYSSTDTFTHLKQVRQLSLLPLMEPIIGVNFAHFLPYGSGQFNSGNRLLGTFGSATLEGVSDYYSQLIYKQNNLSNPPTPPASLPPTPPPMACQKMANGFATTEELAGKAGVLVSHEVTKTLGPKPFQLPFRPQDDLLARALAQGPKTVDVPASLPTPPHNNQEELRIQDHCGDRDTPDSFVPSSSPESVVGVEVSRYPDLSLVKEEPPEPVPSPIIPILPSTAGKSSESRRNDIKTEPGTLYFASPFGPSPNGPRSGLISVAITLHPTAAENISSVVAAFSDLLHVRIPNSYEVSSAPDVPSMGLVSSHRINPGLEYRQHLLLRGPPPGSANPPRLVSSYRLKQPNVPFPPTSNGLSGYKDSSHGIAESAALRPQWCCHCKVVILGSGVRKSFKDLTLLNKDSRESTKRVEKDIVFCSNNCFILYSSTAQAKNSENKESIPSLPQSPMRETPSKAFHQYSNNISTLDVHCLPQLPEKASPPASPPIAFPPAFEAAQVEAKPDELKVTVKLKPRLRAVHGGFEDCRPLNKKWRGMKWKKWSIHIVIPKGTFKPPCEDEIDEFLKKLGTSLKPDPVPKDYRKCCFCHEEGDGLTDGPARLLNLDLDLWVHLNCALWSTEVYETQAGALINVELALRRGLQMKCVFCHKTGATSGCHRFRCTNIYHFTCAIKAQCMFFKDKTMLCPMHKPKGIHEQELSYFAVFRRVYVQRDEVRQIASIVQRGERDHTFRVGSLIFHTIGQLLPQQMQAFHSPKALFPVGYEASRLYWSTRYANRRCRYLCSIEEKDGRPVFVIRIVEQGHEDLVLSDISPKGVWDKILEPVACVRKKSEMLQLFPAYLKGEDLFGLTVSAVARIAESLPGVEACENYTFRYGRNPLMELPLAVNPTGCARSEPKMSAHVKRPHTLNSTSTSKSFQSTVTGELNAPYSKQFVHSKSSQYRKMKTEWKSNVYLARSRIQGLGLYAARDIEKHTMVIEYIGTIIRNEVANRKEKLYESQNRGVYMFRMDNDHVIDATLTGGPARYINHSCAPNCVAEVVTFERGHKIIISSSRRIQKGEELCYDYKFDFEDDQHKIPCHCGAVNCRKWMN

SEQ ID NO: 129 - DNA, 2663 bp, homo sapiens
ggcacgaggcagtgggcaggccaggcagggcgggtacggagcctcccaggctggggcagtgggcatgggcaggggctgtggctgaagacctcgcccgcccactgcagactccaggggactctcacaccgcagctgccatggccaccaataaggagcgactctttgcggctggtgccctggggcctggatctggctacccaggggcaggtttccccttcgccttcccaggggcactcagggggtctccgcctttcgagatgctgagccctagcttccggggcctgggccagcctgacctccccaaggagatggcctctctgtcggtggagacacagagcaccagctcagaggagatggtgcccagctcgccctcgccccctccgcctcctcgggtctacaagccatgcttcgtgtgcaatgacaagtcctctggctaccactatggggtcagctcttgtgaaggctgcaagggcttctttcgccgaagcatccagaagaacatggtgtacacgtgtcaccgcgacaaaaactgtatcatcaacaaggtgaccaggaatcgctgccagtactgccggctacagaagtgcttcgaagtgggcatgtccaaggaagctgtgcgaaatgaccggaacaagaagaagaaagaggtgaaggaagaagggtcacctgacagctatgagctgagccctcagttagaagagctcatcaccaaggtcagcaaagcccatcaggagactttcccctcgctctgccagctgggcaagtataccacgaactccagtgcagaccaccgcgtgcagctggatctggggctgtgggacaagttcagtgagctggctaccaagtgcatcatcaagatcgtggagtttgccaagcggttgcctggctttacagggctcagcattgctgaccagatcactctgctcaaagctgcctgcctagatatcctgatgctgcgtatctgcacaaggtacaccccagagcaggacaccatgaccttctccgacgggctgaccctgaaccggacccagatgcacaatgccggcttcgggcccctcacagaccttgtctttgcctttgctgggcagctcctgcccctggagatggatgacaccgagacagggctgctcagcgccatctgcctcatctgcggagaccgcatggacctggaggagcccgaaaaagtggacaagctgcaggagccactgctggaagccctgaggctgtacgcccggcgccggcggcccagccagccctacatgttcccaaggatgctaatgaaaatcaccgacctccggggcatcagcactaagggagctgaaagggccattactctgaagatggagattccaggcccgatgcctcccttaatccgagagatgctggagaaccctgaaatgtttgaggatgactcctcgcagcctggtccccaccccaatgcctctagcgaggatgaggttcctgggggccagggcaaagggggcctgaagtccccagcctgaccagggcccctgacctccccgctgtgggggttggggcttcaggcagcagactgaccatctcccagaccgccagtgactgggggaggacctgctctgccctctccccaccccttccaatgagctccttgtttttgccaaagtttctaggggtgcctctgtgttcatccccttcctgatctaaccggctccctcgccagtcccgggggcctgccctgctcccaccaggagagagggcaaagggatgagcctgggtttggactctaaaatctcagcactgccccatgggtcctagacttcccagggcaagaggaagaccctgccattccacagccccttcctctgccaggtgcttggctctctgagagcaaacaggaacactagagaccaaaaaggggacaaaggagaagggctgagcccaccttcttgctcctacccttggtgcctaatgctgtgtgatgcacctgcagggtgtgtgctagcctctgtgccccgtccttgtgccaggtcaaggtgggggcaggctgggccctgcatttctggggcaggaacagagggtgaaagggacagatagatgcaggtccattctgcacctcttggctcgggtgcagagttcaccctgtgccctccgttataagtccctcccccagccctgtcatgtgccttgggctcctcctgccctccatctcagccattggggcagggaccctcctacactacagaggggccaggggatccctctctccctagtgccttccaccctttactccccagagcagcttggcccagggaggggggatgctgcttagctgatcccgccctgacccagaggaagcctctatttatttattagcttttgtttacaccgtggaattgaccccttcctccaggggtcttgggtgggggagcccagggcccctgtgacccctcctttcttcctccaatccccagtttgtatttagctgccaaataagattcccattggctccctgtgttctcttggggggtcagggtgctgtcccctcccctctgtttacatctcccctctaccccgctgtatcgcatattgctgagttttctatttttgcaaaataaagtgatggaaactcaaaaaaaaaaaaaaaaaaaaaaaaaaaaaaaaaaaaaaaaaaaaaaaaaaaaaaa

SEQ ID NO: 130 - protein, 454 aa, homo sapiens
MATNKERLFAAGALGPGSGYPGAGFPFAFPGALRGSPPFEMLSPSFRGLGQPDLPKEMASLSVETQSTSSEEMVPSSPSPPPPPRVYKPCFVCNDKSSGYHYGVSSCEGCKGFFRRSIQKNMVYTCHRDKNCIINKVTRNRCQYCRLQKCFEVGMSKEAVRNDRNKKKKEVKEEGSPDSYELSPQLEELITKVSKAHQETFPSLCQLGKYTTNSSADHRVQLDLGLWDKFSELATKCIIKIVEFAKRLPGFTGLSIADQITLLKAACLDILMLRICTRYTPEQDTMTFSDGLTLNRTQMHNAGFGPLTDLVFAFAGQLLPLEMDDTETGLLSAICLICGDRMDLEEPEKVDKLQEPLLEALRLYARRRRPSQPYMFPRMLMKITDLRGISTKGAERAITLKMEIPGPMPPLIREMLENPEMFEDDSSQPGPHPNASSEDEVPGGQGKGGLKSPA

SEQ ID NO: 131 - DNA, 5113 bp, homo sapiens
gctggcggagagcggcctgcgggcgatcgggccgagcctcgctcaaggagcacccccggggcaccctcctgtccgatggcagcccggccctgtccaggaatgtcggtatgacggtctctcagaaagggggtccccagccaacaccgagcccggctggccctgggacgcaactcggaccaatcacaggagagatggatgaagccgattctgcgtttttaaaatttaagcagacagctgatgactctctgtcccttacatctccaaacaccgagtccatttttgtagaagatccctacaccgcctcgctgaggagtgagattgagtcagacggccacgagtttgaagctgagtcctggagcctcgccgtggatgcagcctacgccaagaagcaaaagagggaggtggtgaaaagacaagatgtcctttatgagctgatgcagacagaggtgcaccacgtgcggacgctcaagatcatgctgaaggtgtactccagggccctgcaggaggagctgcagttcagcagcaaggccattggccgcctcttcccatgcgctgacgacctgctggagacgcacagccacttcctcgctcggctcaaggagcgccgccaggagtccctggaggagggcagtgaccggaattatgtcatccagaaaatcggcgacctcctggttcagcagttttcaggtgaaaatggggagagaatgaaagaaaagtacggtgtgttttgtagtggccacaatgaagctgttagtcattacaagttgctgcttcagcaaaacaagaaatttcaaaacttgatcaagaaaattggcaacttctccatcgtgcggcggcttggcgtgcaggagtgcattctcctggttacacaacgcataaccaaatacccagtgctggtggagcgcatcatccagaacacggaagctggcactgaggactatgaagacctgacccaggccttgaacctcatcaaagatatcatctcacaagtggacgccaaggtcagtgagtgtgagaagggccagcgcctcagggagatcgcagggaagatggacctgaagtcttccagcaaactcaagaacgggctcaccttccgcaaggaagacatgcttcagcggcagctccacctggagggcatgctatgctggaagaccacatcagggcgcttgaaagatatcctggctatcctgctgaccgacgtacttttgctgctacaagaaaaagatcagaaatacgtctttgcttctgtggactcaaagccacccgtcatctcgttacaaaagctcatcgtgagggaagtggccaacgaggagaaagcgatgtttctgatcagcgcctccttgcaagggccggagatgtatgaaatctacacgagctccaaagaggacaggaacgcctggatggcccacatccaaagggctgtggagagctgccctgacgaggaggaggggcccttcagcctgcccgaagaggaaaggaaggtggtcgaggcccgcgccacgagactccgggactttcaagagcggttgagcatgaaagaccagctgatcgcacagagcctcctagagaaacagcagatctacctggagatggccgagatgggcggcctcgaagacctgccccagccccgaggcctattccgtggaggggacccatccgagaccctgcagggggagctaattctcaagtcggccatgagcgagatcgagggcatccagagcctgatctgcaggcggctgggcagcgccaacggccaggcggaagacggaggcagctccacaggcccgcccaggagggctgagaccttcgcgggctacgactgcacaaacagccccaccaagaatggcagtttcaagaagaaagtcagcagcactgaccccaggccccgagactggcgaggccccccaaacagcccggacttgaagctcagtgacagtgacattcctgggagctctgaggaatcgccgcaggtggtggaggcgccaggcacggaatccgatccccgtctgcccaccgtcctggagtcggagcttgtccagcggatccagacactgtcccagctgctcctgaaccttcaggcggtaatcgcccaccaggacagctatgtggagacgcagcgggctgccatccaggagcgggagaagcagttccggctgcagtcgacgcgtgggaacctgctgctggagcaggagcggcaacgcaacttcgagaagcagcgggaggagcgcgcggccctggagaagctgcagagccagctgcggcacgagcagcagcgctgggagcgcgagcgccagtggcagcaccaggagctggagcgtgcgggcgcgcggctgcaggagcgcgagggcgaggcgcggcagctacgcgagcggctggagcaggagcgggccgagctggagcgccagcgccaggcctaccagcacgacctggagcggctgcgcgaggcccagcgtgccgtggagcgcgagcgggagcgcctggagctgctgcgccgcctcaagaagcagaacaccgcgccaggcgcgctgccgcccgacacactggccgaggcccagcccccaagccaccctcccagcttcaacggggaagggctggagggccctcgggtgagcatgctgccatccggcgtggggccagagtacgcagagcgccccgaggtggctcgccgggacagcgcccccaccgagagccggctggccaagagcgatgtgcccatccagctgctcagcgccaccaaccagttccagaggcaggcggccgtgcagcagcagatccccaccaagctggcggcctccaccaagggtggcaaggacaagggcggcaagagcaggggctctcagcgctgggagagctcagcgtccttcgacctgaagcagcagctgctgctcaacaagctcatggggaaagatgagagcacctcacggaaccgccgctcgctgagccctatcctgcccggcagacacagtcctgcgcccccaccagaccctggcttccccgccccgagcccaccgccagctgacagcccctccgagggcttctctctcaaggccgggggcacagccctcctgcccggccccccagctccctcgccactgccggccacaccactcagcgccaaggaggacgccagcaaagaagacgtcatcttcttctaaaagggccgtgactcaaggaaagtttttaatggaaagttgagccagaactaaaccagggagctgtctgaaatcatagcaccccatccgggtggcggggagatcaactccgagctgtttttccgaggcagtgaggaacggtgccggctctgcacggagctgaggacaggacagaccttgctttgagaaggagctgccggccggggccacgctccacagccgccgcgcgacagtggagccaagggttagggcaccaggaggggccaggtggcgtcggcagcatctgtccccagaatcaggcagaatccacttcccaaacagagccccacgcaggttcaccatgaacctcagggtcagggaatgagccaggcacgggggcatgggcagagagggccacggggcagggcccactgagggaacatcagtggccctccagtcaggttctgtgggtttggaagcccatcgtgaaaggggctgacctttgcccctttttacttggcattggttttgaaaccagctgtttcccaaactctgcttcccaagggcaaccgttgctgttcacacgctcagcctgtctgggggagcgggcctctagcttcagccagggcgggtacacaccctgggcacagggtcctcagcccccgggaaatgagctcccagggctggcgtcccaccttccaggtgggggctggcacatcacagactgtcgagagcgccatgtcccagggcatgcagaggttgcacctagagacgttgcagcaagtggacaagtggccgctgtgcgggcccctcgcttgtagtgagctgttgcagcttacggtccgttccctggaggggtggaggaaggaggtgttgggcagcatcaaaggtgctgggacatcccagggtggtgagatccatccacgatccagctccggtggagaaagggcccatgtcaagccttgttctgcaccccaagcattggtggtaggactgggtcctggctgatcgtccttgttcccagtggggtacatgtgagcccctgccagggccaagtccttctcccgaacccagggtcctgggaactgcagatcccggggggattcagcccttctcccactgtgctggcagaggcactcctgtgacgctgaatacagtgaacagggacattcccgccactcggggacagatgggcacaagggaggggaaactccatcaggaagtgctcccctgggcagaggcgcccactgggtgctgtgggctcaggagggggcggggcaggagctggtgccaaccgggaaccagagccccacagccatacagcccattggtgacaaggtcctgagaacacagtggccaggtgtccccaggctcctggcccctccgacgacctcaactctgcccagcccggtccctggccatcagcgacgctgtccgccccccgtcagatcccatgtgtgccatgtttatcatcagtgttttgtatttttgtactgagtatcggagcactttacagaagctgactgtacattcctgttctgttgtgaagagaacattcccagaccctggcaccctcctgagccggcgtgtgccggtccagccctccgagatgccacaattccttggatgggggagaagttcaaggaatttctgctcggccacgcggtgggaaccccgcgtccccgccatgtggcagaggggtctcagtcgtgctaggcatcgggcggcagcgccgacagcccttccctcgccagtgcccctcggccactcctgggttggagcccgattttatttgtaaagttgacagtcgagcaaatgttcctattttcgtgggatctgcacacgtctttgtcagttgtggtcatgatcttagtcacctgctaattatttttacaatgattacaacatttcctcactgcgggatatttctgacccgctttagaacttaagacctgattctagcaataaacgtgtccgagatg

SEQ ID NO: 132 - protein, 1015 aa, homo sapiens
MTVSQKGGPQPTPSPAGPGTQLGPITGEMDEADSAFLKFKQTADDSLSLTSPNTESIFVEDPYTASLRSEIESDGHEFEAESWSLAVDAAYAKKQKREVVKRQDVLYELMQTEVHHVRTLKIMLKVYSRALQEELQFSSKAIGRLFPCADDLLETHSHFLARLKERRQESLEEGSDRNYVIQKIGDLLVQQFSGENGERMKEKYGVFCSGHNEAVSHYKLLLQQNKKFQNLIKKIGNFSIVRRLGVQECILLVTQRITKYPVLVERIIQNTEAGTEDYEDLTQALNLIKDIISQVDAKVSECEKGQRLREIAGKMDLKSSSKLKNGLTFRKEDMLQRQLHLEGMLCWKTTSGRLKDILAILLTDVLLLLQEKDQKYVFASVDSKPPVISLQKLIVREVANEEKAMFLISASLQGPEMYEIYTSSKEDRNAWMAHIQRAVESCPDEEEGPFSLPEEERKVVEARATRLRDFQERLSMKDQLIAQSLLEKQQIYLEMAEMGGLEDLPQPRGLFRGGDPSETLQGELILKSAMSEIEGIQSLICRRLGSANGQAEDGGSSTGPPRRAETFAGYDCTNSPTKNGSFKKKVSSTDPRPRDWRGPPNSPDLKLSDSDIPGSSEESPQVVEAPGTESDPRLPTVLESELVQRIQTLSQLLLNLQAVIAHQDSYVETQRAAIQEREKQFRLQSTRGNLLLEQERQRNFEKQREERAALEKLQSQLRHEQQRWERERQWQHQELERAGARLQEREGEARQLRERLEQERAELERQRQAYQHDLERLREAQRAVERERERLELLRRLKKQNTAPGALPPDTLAEAQPPSHPPSFNGEGLEGPRVSMLPSGVGPEYAERPEVARRDSAPTESRLAKSDVPIQLLSATNQFQRQAAVQQQIPTKLAASTKGGKDKGGKSRGSQRWESSASFDLKQQLLLNKLMGKDESTSRNRRSLSPILPGRHSPAPPPDPGFPAPSPPPADSPSEGFSLKAGGTALLPGPPAPSPLPATPLSAKEDASKEDVIFF

SEQ ID NO: 133 - DNA, 1595 bp, homo sapiens
cccgggccggggcacggagagagccgagcgccgcagccgtgagccgaatagagccggagagacccgagtatgaccggagaagcccaggccggccggaagaggagccgagcgcggccggaaggaaccgagcccgtccgaagggagcggagcgcagcctggcctggggcccggtcgagcccgcgccatggcggccgaggcgacagctgtggccggaagcggggctgttggcggctgcctggccaaagacggcttgcagcagtctaagtgcccggacactaccccaaaacggcggcgcgcctcgtcgctgtcgcgtgacgccgagcgccgagcctaccaatggtgccgggagtacttgggcggggcctggcgccgagtgcagcccgaggagctgagggtttaccccgtgagcggaggcctcagcaacctgctcttccgctgctcgctcccggaccacctgcccagcgttggcgaggagccccgggaggtgcttctgcggctgtacggagccatcttgcagggcgtggactccctggtgctagaaagcgtgatgttcgccatacttgcggagcggtcgctggggccccagctgtacggagtcttcccagagggccggctggaacagtacatcccaagtcggccattgaaaactcaagagcttcgagagccagtgttgtcagcagccattgccacgaagatggcgcaatttcatggcatggagatgcctttcaccaaggagccccactggctgtttgggaccatggagcggtacctaaaacagatccaggacctgcccccaactggcctccctgagatgaacctgctggagatgtacagcctgaaggatgagatgggcaacctcaggaagttactagagtctaccccatcgccagtcgtcttctgccacaatgacatccaggaagggaacatcttgctgctctcagagccagaaaatgctgacagcctcatgctggtggacttcgagtacagcagttataactataggggctttgacattgggaaccatttttgtgagtgggtttatgattatactcacgaggaatggcctttctacaaagcaaggcccacagactaccccactcaagaacagcagttgcattttattcgtcattacctggcagaggcaaagaaaggtgagaccctctcccaagaggagcagagaaaactggaagaagatttgctggtagaagtcagtcggtatgctctggcatcccatttcttctggggtctgtggtccatcctccaggcatccatgtccaccatagaatttggttacttggactatgcccagtctcggttccagttctacttccagcagaaggggcagctgaccagtgtccactcctcatcctgactccaccctcccactccttggatttctcctggagcctccagggcaggaccttggagggaggaacaacgagcagaaggccctggcgactgggctgagcccccaagtgaaactgaggttcaggagaccggcctgttcctgagtttgagtaggtccccatggctggcaggccagagccccgtgctgtgtatgtaacacaataaacaagcttcttcttcccaccctg

SEQ ID NO: 134 - protein, 395 aa, homo sapiens
MAAEATAVAGSGAVGGCLAKDGLQQSKCPDTTPKRRRASSLSRDAERRAYQWCREYLGGAWRRVQPEELRVYPVSGGLSNLLFRCSLPDHLPSVGEEPREVLLRLYGAILQGVDSLVLESVMFAILAERSLGPQLYGVFPEGRLEQYIPSRPLKTQELREPVLSAAIATKMAQFHGMEMPFTKEPHWLFGTMERYLKQIQDLPPTGLPEMNLLEMYSLKDEMGNLRKLLESTPSPVVFCHNDIQEGNILLLSEPENADSLMLVDFEYSSYNYRGFDIGNHFCEWVYDYTHEEWPFYKARPTDYPTQEQQLHFIRHYLAEAKKGETLSQEEQRKLEEDLLVEVSRYALASHFFWGLWSILQASMSTIEFGYLDYAQSRFQFYFQQKGQLTSVHSSS

SEQ ID NO: 135 - DNA, 1362 bp, homo sapiens
catttggggacgctctcagctctcggcgcacggcccagcttccttcaaaatgtctactgttcacgaaatcctgtgcaagctcagcttggagggtgatcactctacacccccaagtgcatatgggtctgtcaaagcctatactaactttgatgctgagcgggatgctttgaacattgaaacagccatcaagaccaaaggtgtggatgaggtcaccattgtcaacattttgaccaaccgcagcaatgcacagagacaggatattgccttcgcctaccagagaaggaccaaaaaggaacttgcatcagcactgaagtcagccttatctggccacctggagacggtgattttgggcctattgaagacacctgctcagtatgacgcttctgagctaaaagcttccatgaaggggctgggaaccgacgaggactctctcattgagatcatctgctccagaaccaaccaggagctgcaggaaattaacagagtctacaaggaaatgtacaagactgatctggagaaggacattatttcggacacatctggtgacttccgcaagctgatggttgccctggcaaagggtagaagagcagaggatggctctgtcattgattatgaactgattgaccaagatgctcgggatctctatgacgctggagtgaagaggaaaggaactgatgttcccaagtggatcagcatcatgaccgagcggagcgtgccccacctccagaaagtatttgataggtacaagagttacagcccttatgacatgttggaaagcatcaggaaagaggttaaaggagacctggaaaatgctttcctgaacctggttcagtgcattcagaacaagcccctgtattttgctgatcggctgtatgactccatgaagggcaaggggacgcgagataaggtcctgatcagaatcatggtctcccgcagtgaagtggacatgttgaaaattaggtctgaattcaagagaaagtacggcaagtccctgtactattatatccagcaagacactaagggcgactaccagaaagcgctgctgtacctgtgtggtggagatgactgaagcccgacacggcctgagcgtccagaaatggtgctcaccatgcttccagctaacaggtctagaaaaccagcttgcgaataacagtccccgtggccatccctgtgagggtgacgttagcattacccccaacctcattttagttgcctaagcattgcctggccttcctgtctagtctctcctgtaagccaaagaaatgaacattccaaggagttggaagtgaagtctatgatgtgaaacactttgcctcctgtgtactgtgtcataaacagatgaataaactgaatttgtacttt

SEQ ID NO: 136 - protein, 339 aa, homo sapiens
MSTVHEILCKLSLEGDHSTPPSAYGSVKAYTNFDAERDALNIETAIKTKGVDEVTIVNILTNRSNAQRQDIAFAYQRRTKKELASALKSALSGHLETVILGLLKTPAQYDASELKASMKGLGTDEDSLIEIICSRTNQELQEINRVYKEMYKTDLEKDIISDTSGDFRKLMVALAKGRRAEDGSVIDYELIDQDARDLYDAGVKRKGTDVPKWISIMTERSVPHLQKVFDRYKSYSPYDMLESIRKEVKGDLENAFLNLVQCIQNKPLYFADRLYDSMKGKGTRDKVLIRIMVSRSEVDMLKIRSEFKRKYGKSLYYYIQQDTKGDYQKALLYLCGGDD

SEQ ID NO: 137 - DNA, 1982 bp, homo sapiens
tactatcagggggcaagagcctttctctccagctacacactccatctcccgggagcaaggggaaactccgagaggagggcaacagagccagcatcttgccagggccccggaggaggggttccccgctacgcctgtgccggaggagttccagtcaccgagcgaggggcgcaagggtgggtgcatcctgcgctgcggcgggcgcgctacccagacgctggtgtgcagagccacatgaagcctgctggggactgggggccagggagcagcaagccagctgggactgaggcggacgctgtctcagggagacgctgactcgcaaagacactcccttccttgtgcctgggtaaaaagtctcctcctggggtccctggccatcctgaatatccagaatggtgtttctgaagttcttctgcatgagtttcttctgccacctgtgtcaaggctacttcgatggccccctctacccagagatgtccaatgggactctgcaccactacttcgtgcccgatggggactatgaggagaacgatgaccccgagaagtgccagctgctcttcagggtgagtgaccacaggcgctgctcccagggggaggggagccaggttggcagcctgctgagcctcaccctgcgggaggagttcaccgtgctgggccgccaggtggaggatgctgggcgcgtgctggagggcatcagcaaaagcatctcctacgacctagacggggaagagagctatggcaagtacctgcggcgggagtcccaccagatcggggatgcctactccaactcggacaaatccctcactgagctggagagcaagttcaagcagggccaggaacaggacagccggcaggagagcaggctcaacgaggactttctgggaatgctggtccacaccaggtccctgctgaaggagacactggacatctctgtggggctcagggacaaatacgagctgctggccctcaccattaggagccatgggacccgactaggtcggctgaaaaatgattatcttaaagtataggtggaaggatacaaatgctagaaagagggaatcaaatcagccccgttttggagggtgggggacagaagatggggctacatttcccccatacctactatttttttatatcccgatttgcactttgagaatacatctaaggtcatctttcaaaagagaaaaattggacacttgagtgactttgtttttagttttgtttttgtacattatttatgtgattgttatggaattgtcacctggaaagaacaattttaagcaatgtcatttctagatgggtttctaattctgcagagacacccgtttcagccacatctaaaagagcacagtttatgtggtgcggaattaaacttccccatcctgcagattatgtggaaatacccaaagataatagtgcatagctcctttcagcctctagccttcactcctgggctccaaaagctatcccagttgcctgtttttcaaatgaggttcaaggtgctgctttgcatgcctgccaacccatggaagttgtttcttacttcttttctctcttatttattaaccatggtctgagagttgtttttgttctatgtaacagtattgccacaaaactataggcaaatcgtgtttgcagggagatttctgatgcctctgtgggtgtgtgtaagttaaagtggccacatttaagaaggccaagctttgtagtggttgcacagtcacactgatatgctgatttgctctttctcattgtatgtctatgctttgtcatcagtgctatagtaaattacaaagaaataggtagattgtatgaacatacccacaaatgcctatgatttaggttaccaatgtattctttctcatttggggttttgcttctgtctgtctgtttattggaaacttgtacttcaagtagggggaatcctaattctaataactccttagctaagttttattattcaggcaataaacatgttttcatgt

SEQ ID NO: 138 - protein, 211 aa, homo sapiens
MVFLKFFCMSFFCHLCQGYFDGPLYPEMSNGTLHHYFVPDGDYEENDDPEKCQLLFRVSDHRRCSQGEGSQVGSLLSLTLREEFTVLGRQVEDAGRVLEGISKSISYDLDGEESYGKYLRRESHQIGDAYSNSDKSLTELESKFKQGQEQDSRQESRLNEDFLGMLVHTRSLLKETLDISVGLRDKYELLALTIRSHGTRLGRLKNDYLKV

SEQ ID NO: 139 - DNA, 2574 bp, homo sapiens
cctgtttagacacatggacaacaatcccagcgctacaaggcacacagtccgcttcttcgtcctcagggttgccagcgcttcctggaagtcctgaagctctcgcagtgcagtgagttcatgcaccttcttgccaagcctcagtctttgggatctggggaggccgcctggttttcctccctccttctgcacgtctgctggggtctcttcctctccaggccttgccgtccccctggcctctcttcccagctcacacatgaagatgcacttgcaaagggctctggtggtcctggccctgctgaactttgccacggtcagcctctctctgtccacttgcaccaccttggacttcggccacatcaagaagaagagggtggaagccattaggggacagatcttgagcaagctcaggctcaccagcccccctgagccaacggtgatgacccacgtcccctatcaggtcctggccctttacaacagcacccgggagctgctggaggagatgcatggggagagggaggaaggctgcacccaggaaaacaccgagtcggaatactatgccaaagaaatccataaattcgacatgatccaggggctggcggagcacaacgaactggctgtctgccctaaaggaattacctccaaggttttccgcttcaatgtgtcctcagtggagaaaaatagaaccaacctattccgagcagaattccgggtcttgcgggtgcccaaccccagctctaagcggaatgagcagaggatcgagctcttccagatccttcggccagatgagcacattgccaaacagcgctatatcggtggcaagaatctgcccacacggggcactgccgagtggctgtcctttgatgtcactgacactgtgcgtgagtggctgttgagaagagagtccaacttaggtctagaaatcagcattcactgtccatgtcacacctttcagcccaatggagatatcctggaaaacattcacgaggtgatggaaatcaaattcaaaggcgtggacaatgaggatgaccatggccgtggagatctggggcgcctcaagaagcagaaggatcaccacaaccctcatctaatcctcatgatgattcccccacaccggctcgacaacccgggccaggggggtcagaggaagaagcgggctttggacaccaattactgcttccgcaacttggaggagaactgctgtgtgcgccccctctacattgacttccgacaggatctgggctggaagtgggtccatgaacctaagggctactatgccaacttctgctcaggcccttgcccatacctccgcagtgcagacacaacccacagcacggtgctgggactgtacaacactctgaaccctgaagcatctgcctcgccttgctgcgtgccccaggacctggagcccctgaccatcctgtactatgttgggaggacccccaaagtggagcagctctccaacatggtggtgaagtcttgtaaatgtagctgagaccccacgtgcgacagagagaggggagagagaaccaccactgcctgactgcccgctcctcgggaaacacacaagcaacaaacctcactgagaggcctggagcccacaaccttcggctccgggcaaatggctgagatggaggtttccttttggaacatttctttcttgctggctctgagaatcacggtggtaaagaaagtgtgggtttggttagaggaaggctgaactcttcagaacacacagactttctgtgacgcagacagaggggatggggatagaggaaagggatggtaagttgagatgttgtgtggcaatgggatttgggctaccctaaagggagaaggaagggcagagaatggctgggtcagggccagactggaagacacttcagatctgaggttggatttgctcattgctgtaccacatctgctctagggaatctggattatgttatacaaggcaagcatttttttttttaaagacaggttacgaagacaaagtcccagaattgtatctcatactgtctgggattaagggcaaatctattacttttgcaaactgtcctctacatcaattaacatcgtgggtcactacagggagaaaatccaggtcatgcagttcctggcccatcaactgtattgggccttttggatatgctgaacgcagaagaaagggtggaaatcaaccctctcctgtctgccctctgggtccctcctctcacctctccctcgatcatatttccccttggacacttggttagacgccttccaggtcaggatgcacatttctggattgtggttccatgcagccttggggcattatgggtcttcccccacttcccctccaagaccctgtgttcatttggtgttcctggaagcaggtgctacaacatgtgaggcattcggggaagctgcacatgtgccacacagtgacttggccccagacgcatagactgaggtataaagacaagtatgaatattactctcaaaatctttgtataaataaatatttttggggcatcctggatgatttcatcttctggaatattgtttctagaacagtaaaagccttattctaaggtg

SEQ ID NO: 140 - protein, 412 aa, homo sapiens
MKMHLQRALVVLALLNFATVSLSLSTCTTLDFGHIKKKRVEAIRGQILSKLRLTSPPEPTVMTHVPYQVLALYNSTRELLEEMHGEREEGCTQENTESEYYAKEIHKFDMIQGLAEHNELAVCPKGITSKVFRFNVSSVEKNRTNLFRAEFRVLRVPNPSSKRNEQRIELFQILRPDEHIAKQRYIGGKNLPTRGTAEWLSFDVTDTVREWLLRRESNLGLEISIHCPCHTFQPNGDILENIHEVMEIKFKGVDNEDDHGRGDLGRLKKQKDHHNPHLILMMIPPHRLDNPGQGGQRKKRALDTNYCFRNLEENCCVRPLYIDFRQDLGWKWVHEPKGYYANFCSGPCPYLRSADTTHSTVLGLYNTLNPEASASPCCVPQDLEPLTILYYVGRTPKVEQLSNMVVKSCKCS

SEQ ID NO: 141 - DNA, 3603 bp, homo sapiens
acaaagggaggaggaagaagggagcggggtcggagccgtcggggccaaaggagacggggccaggaacaggcagtctcggcccaactgcggacgctccctccaccccctgcgcaaaaagacccaaccggagttgaggcgctgcccctgaaggccccaccttacacttggcgggggccggagccaggctcccaggactgctccagaaccgagggaagctcgggtccctccaagctagccatggtgaggcgccggaggccccggggccccacccccccggcctgaccacactgccctgggtgccctcctccagaagcccgagatgcggggggccgggagacaacactcctggctccccagagaggcgtgggtctggggctgagggccagggcccggatgcccaggttccgggactagggccttggcagccagcgggggtggggaccacgggcacccagagaaggtcctccacacatcccagcgccggctcccggccatggagcccttgaagagcctcttcctcaagagccctctagggtcatggaatggcagtggcagcgggggtggtgggggcggtggaggaggccggcctgaggggtctccaaaggcagcgggttatgccaacccggtgtggacagccctgttcgactacgagcccagtgggcaggatgagctggccctgaggaagggtgaccgtgtggaggtgctgtcccgggacgcagccatctcaggagacgagggctggtgggcgggccaggtgggtggccaggtgggcatcttcccgtccaactatgtgtctcggggtggtggcccgcccccctgcgaggtggccagcttccaggagctgcggctggaggaggtgatcggcattggaggctttggcaaggtgtacaggggcagctggcgaggtgagctggtggctgtgaaggcagctcgccaggaccccgatgaggacatcagtgtgacagccgagagcgttcgccaggaggcccggctcttcgccatgctggcacaccccaacatcattgccctcaaggctgtgtgcctggaggagcccaacctgtgcctggtgatggagtatgcagccggtgggcccctcagccgagctctggccgggcggcgcgtgcctccccatgtgctggtcaactgggctgtgcagattgcccgtgggatgcactacctgcactgcgaggccctggtgcccgtcatccaccgtgatctcaagtccaacaacattttgctgctgcagcccattgagagtgacgacatggagcacaagaccctgaagatcaccgactttggcctggcccgagagtggcacaaaaccacacaaatgagtgccgcgggcacctacgcctggatggctcctgaggttatcaaggcctccaccttctctaagggcagtgacgtctggagttttggggtgctgctgtgggaactgctgaccggggaggtgccataccgtggcattgactgccttgctgtggcctatggcgtagctgttaacaagctcacactgcccatcccatccacctgccccgagcccttcgcacagcttatggccgactgctgggcgcaggacccccaccgcaggcccgacttcgcctccatcctgcagcagttggaggcgctggaggcacaggtcctacgggaaatgccgcgggactccttccattccatgcaggaaggctggaagcgcgagatccagggtctcttcgacgagctgcgagccaaggaaaaggaactactgagccgcgaggaggagctgacgcgagcggcgcgcgagcagcggtcacaggcggagcagctgcggcggcgcgagcacctgctggcccagtgggagctagaggtgttcgagcgcgagctgacgctgctgctgcagcaggtggaccgcgagcgaccgcacgtgcgccgccgccgcgggacattcaagcgcagcaagctccgggcgcgcgacggcggcgagcgtatcagcatgccactcgacttcaagcaccgcatcaccgtgcaggcctcacccggccttgaccggaggagaaacgtcttcgaggtcgggcctggggattcgcccacctttccccggttccgagccatccagttggagcctgcagagccaggccaggcatggggccgccagtccccccgacgtctggaggactcaagcaatggagagcggcgagcatgctgggcttggggtcccagttcccccaagcctggggaagcccagaatgggaggagaaggtcccgcatggacgaagccacatggtacctggattcagatgactcatcccccttaggatctccttccacacccccagcactcaatggtaaccccccgcggcctagcctggagcccgaggagcccaagaggcctgtccccgcagagcgcggtagcagctctgggacgcccaagctgatccagcgggcgctgctgcgcggcaccgccctgctcgcctcgctgggccttggccgcgacctgcagccgccgggaggcccaggacgcgagcgcggggagtccccgacaacaccccccacgccaacgcccgcgccctgcccgaccgagccgcccccttccccgctcatctgcttctcgctcaagacgcccgactccccgcccactcctgcacccctgttgctggacctgggtatccctgtgggccagcggtcagccaagagcccccgacgtgaggaggagccccgcggaggcactgtctcacccccaccggggacatcacgctctgctcctggcaccccaggcaccccacgttcaccacccctgggcctcatcagccgacctcggccctcgccccttcgcagccgcattgatccctggagctttgtgtcagctgggccacggccttctcccctgccatcaccacagcctgcaccccgccgagcaccctggaccttgttcccggactcagaccccttctgggactccccacctgccaaccccttccaggggggcccccaggactgcagggcacagaccaaagacatgggtgcccaggccccgtgggtgccggaagcggggccttgagtgggccaggccactcccccgagctccagctgccttaggaggagtcacagcatacactggaacaggagctgggtcagcctctgcagctgcctcagtttccccagggaccccacccccctttgggggtcaggaacactacactgcacaggaagccttcacactggaagggggacctgcgcccccacatctgaaacctgtaggtccccccagctcacctgccctactggggcccaacactgtacccagctggttgggaggaccagagcctgtctcagggaattgcctgctggggtgatgcagggaggaggggaggtgcagggaagaggggccggcctcagctgtcaccagcacttttgaccaagtcctgctactgcggcccctgccctagggcttagagcatggacctcctgccctgggggtcatctggggccagggctctctggatgccttcctgctgccccagccagggttggagtcttagcctcgggatccagtgaagccagaagccaaataaactcaaaagctgtctccccaaaaaaaaaaaaaaaaaaaaaaaaaaaaaaaaa

SEQ ID NO: 142 - protein, 847 aa, homo sapiens
MEPLKSLFLKSPLGSWNGSGSGGGGGGGGGRPEGSPKAAGYANPVWTALFDYEPSGQDELALRKGDRVEVLSRDAAISGDEGWWAGQVGGQVGIFPSNYVSRGGGPPPCEVASFQELRLEEVIGIGGFGKVYRGSWRGELVAVKAARQDPDEDISVTAESVRQEARLFAMLAHPNIIALKAVCLEEPNLCLVMEYAAGGPLSRALAGRRVPPHVLVNWAVQIARGMHYLHCEALVPVIHRDLKSNNILLLQPIESDDMEHKTLKITDFGLAREWHKTTQMSAAGTYAWMAPEVIKASTFSKGSDVWSFGVLLWELLTGEVPYRGIDCLAVAYGVAVNKLTLPIPSTCPEPFAQLMADCWAQDPHRRPDFASILQQLEALEAQVLREMPRDSFHSMQEGWKREIQGLFDELRAKEKELLSREEELTRAAREQRSQAEQLRRREHLLAQWELEVFERELTLLLQQVDRERPHVRRRRGTFKRSKLRARDGGERISMPLDFKHRITVQASPGLDRRRNVFEVGPGDSPTFPRFRAIQLEPAEPGQAWGRQSPRRLEDSSNGERRACWAWGPSSPKPGEAQNGRRRSRMDEATWYLDSDDSSPLGSPSTPPALNGNPPRPSLEPEEPKRPVPAERGSSSGTPKLIQRALLRGTALLASLGLGRDLQPPGGPGRERGESPTTPPTPTPAPCPTEPPPSPLICFSLKTPDSPPTPAPLLLDLGIPVGQRSAKSPRREEEPRGGTVSPPPGTSRSAPGTPGTPRSPPLGLISRPRPSPLRSRIDPWSFVSAGPRPSPLPSPQPAPRRAPWTLFPDSDPFWDSPPANPFQGGPQDCRAQTKDMGAQAPWVPEAGP

SEQ ID NO: 143 - DNA, 1571 bp, homo sapiens
aaggtgagcgactgcaggcaaacccggcgacagcgcagctcgcgtcgaccctggctcctctgcctgccccctcaggcccccgcctccttcaggatgacgctggacgtggggccggaggatgagctgcccgactgggccgccgccaaagagttttaccagaagtacgaccctaaggacgtcatcggcagaggagtgagctctgtggtccgccgttgtgttcatcgagctactggccacgagtttgcggtgaagattatggaagtgacagctgagcggctgagtcctgagcagctggaggaggtgcgggaagccacacggcgagagacacacatccttcgccaggtcgccggccacccccacatcatcaccctcatcgattcctacgagtcttctagcttcatgttcctggtgtttgacctgatgcggaagggagagctgtttgactatctcacagagaaggtggccctctctgaaaaggaaaccaggtccatcatgcggtctctgctggaagcagtgagctttctccatgccaacaacattgtgcatcgagatctgaagcccgagaatattctcctagatgacaatatgcagatccgactttcagatttcgggttctcctgccacttggaacctggcgagaagcttcgagagttgtgtgggaccccagggtatctagcgccagagatccttaaatgctccatggatgaaacccacccaggctatggcaaggaggtcgacctctgggcctgtggggtgatcttgttcacactcctggctggctcgccacccttctggcaccggcggcagatcctgatgttacgcatgatcatggagggccagtaccagttcagttcccccgagtgggatgaccgttccagcactgtcaaagacctgatctccaggctgctgcaggtggatcctgaggcacgcctgacagctgagcaggccctacagcaccccttctttgagcgttgtgaaggcagccaaccctggaacctcaccccccgccagcggttccgggtggcagtgtggacagtgctggctgctggacgagtggccctaagcacccatcgtgtacggccactgaccaagaatgcactgttgagggacccttatgcgctgcggtcagtgcggcacctcatcgacaactgtgccttccggctctacgggcactgggtaaagaaaggggagcagcagaaccgggcggctctctttcagcaccggccccctgggccttttcccatcatgggccctgaagaggagggagactctgctgctataactgaggatgaggccgtgcttgtgctgggctaggacctcaaccccagggattcccaggaagcagaactctccagaagaagggttttgatcattccagctcctctgggctctggcctcaggcccactaatgatcctgctaccctcttgaagaccagcccggtacctctctccccactggccaggactctgagatcagagctggggtggaagggagccattctgaacgccacgcctggcccggtcagtgctgcatgcactgcatatgaaataaaatctgctacacgccaggg

SEQ ID NO: 144 - protein, 406 aa, homo sapiens
MTLDVGPEDELPDWAAAKEFYQKYDPKDVIGRGVSSVVRRCVHRATGHEFAVKIMEVTAERLSPEQLEEVREATRRETHILRQVAGHPHIITLIDSYESSSFMFLVFDLMRKGELFDYLTEKVALSEKETRSIMRSLLEAVSFLHANNIVHRDLKPENILLDDNMQIRLSDFGFSCHLEPGEKLRELCGTPGYLAPEILKCSMDETHPGYGKEVDLWACGVILFTLLAGSPPFWHRRQILMLRMIMEGQYQFSSPEWDDRSSTVKDLISRLLQVDPEARLTAEQALQHPFFERCEGSQPWNLTPRQRFRVAVWTVLAAGRVALSTHRVRPLTKNALLRDPYALRSVRHLIDNCAFRLYGHWVKKGEQQNRAALFQHRPPGPFPIMGPEEEGDSAAITEDEAVLVLG

SEQ ID NO: 145 - DNA, 952 bp, homo sapiens
tgaactctggatgctgttagcctgagactcaggaagacaacttctgcagggtcactccctggcttctggaggaaagagaaggagggcagtgctccagtggtacagaagtgagacataatggaatcaggcttcacctccaaggacacctatctaagccattttaaccctcgggattacctagaaaaatattacaagtttggttctaggcactctgcagaaagccagattcttaagcaccttctgaaaaatcttttcaagatattctgcctagacggtgtgaagggagacctgctgattgacatcggctctggccccactatctatcagctcctctctgcttgtgaatcctttaaggagatcgtcgtcactgactactcagaccagaacctgcaggagctggagaagtggctgaagaaagagccagaggcctttgactggtccccagtggtgacctatgtgtgtgatcttgaagggaacagagtcaagggtccagagaaggaggagaagttgagacaggcggtcaagcaggtgctgaagtgtgatgtgactcagagccagccactgggggccgtccccttacccccggctgactgcgtgctcagcacactgtgtctggatgccgcctgcccagacctccccacctactgcagggcgctcaggaacctcggcagcctactgaagccagggggcttcctggtgatcatggatgcgctcaagagcagctactacatgattggtgagcagaagttctccagcctccccctgggccgggaggcagtagaggctgctgtgaaagaggctggctacacaatcgaatggtttgaggtgatctcgcaaagttattcttccaccatggccaacaacgaaggacttttctccctggtggcgaggaagctgagcagacccctgtgatgcctgtgacctcaattaaagcaattcctttgacctgtca

SEQ ID NO: 146 - protein, 264 aa, homo sapiens
MESGFTSKDTYLSHFNPRDYLEKYYKFGSRHSAESQILKHLLKNLFKIFCLDGVKGDLLIDIGSGPTIYQLLSACESFKEIVVTDYSDQNLQELEKWLKKEPEAFDWSPVVTYVCDLEGNRVKGPEKEEKLRQAVKQVLKCDVTQSQPLGAVPLPPADCVLSTLCLDAACPDLPTYCRALRNLGSLLKPGGFLVIMDALKSSYYMIGEQKFSSLPLGREAVEAAVKEAGYTIEWFEVISQSYSSTMANNEGLFSLVARKLSRPL

SEQ ID NO: 147 - DNA, 830 bp, homo sapiens
cccccccgagcgccgctccggctgcaccgcgctcgctccgagtttcaggctcgtgctaagctagcgccgtcgtcgtctcccttcagtcgccatcatgattatctaccgggacctcatcagccacgatgagatgttctccgacatctacaagatccgggagatcgcggacgggttgtgcctggaggtggaggggaagatggtcagtaggacagaaggtaacattgatgactcgctcattggtggaaatgcctccgctgaaggccccgagggcgaaggtaccgaaagcacagtaatcactggtgtcgatattgtcatgaaccatcacctgcaggaaacaagtttcacaaaagaagcctacaagaagtacatcaaagattacatgaaatcaatcaaagggaaacttgaagaacagagaccagaaagagtaaaaccttttatgacaggggctgcagaacaaatcaagcacatccttgctaatttcaaaaactaccagttctttattggtgaaaacatgaatccagatggcatggttgctctattggactaccgtgaggatggtgtgaccccatatatgattttctttaaggatggtttagaaatggaaaaatgttaacaaatgtggcaattattttggatctatcacctgtcatcataactggcttctgcttgtcatccacacaacaccaggacttaagacaaatgggactgatgtcatcttgagctcttcatttattttgactgtgatttatttggagtggaggcattgtttttaagaaaaacatgtcatgtaggttgtctaaaaataaaatgcatttaaactcatttgagag

SEQ ID NO: 148 - protein, 172 aa, homo sapiens
MIIYRDLISHDEMFSDIYKIREIADGLCLEVEGKMVSRTEGNIDDSLIGGNASAEGPEGEGTESTVITGVDIVMNHHLQETSFTKEAYKKYIKDYMKSIKGKLEEQRPERVKPFMTGAAEQIKHILANFKNYQFFIGENMNPDGMVALLDYREDGVTPYMIFFKDGLEMEKC

SEQ ID NO: 149 - DNA, 2077 bp, homo sapiens
agcgcagcgtgcgggtggcctggatcccgcgcagtggcccggcgatgtcgctcgtgctgctaagcctggccgcgctgtgcaggagcgccgtaccccgagagccgaccgttcaatgtggctctgaaactgggccatctccagagtggatgctacaacatgatctaatccccggagacttgagggacctccgagtagaacctgttacaactagtgttgcaacaggggactattcaattttgatgaatgtaagctgggtactccgggcagatgccagcatccgcttgttgaaggccaccaagatttgtgtgacgggcaaaagcaacttccagtcctacagctgtgtgaggtgcaattacacagaggccttccagactcagaccagaccctctggtggtaaatggacattttcctacatcggcttccctgtagagctgaacacagtctatttcattggggcccataatattcctaatgcaaatatgaatgaagatggcccttccatgtctgtgaatttcacctcaccaggctgcctagaccacataatgaaatataaaaaaaagtgtgtcaaggccggaagcctgtgggatccgaacatcactgcttgtaagaagaatgaggagacagtagaagtgaacttcacaaccactcccctgggaaacagatacatggctcttatccaacacagcactatcatcgggttttctcaggtgtttgagccacaccagaagaaacaaacgcgagcttcagtggtgattccagtgactggggatagtgaaggtgctacggtgcagctgactccatattttcctacttgtggcagcgactgcatccgacataaaggaacagttgtgctctgcccacaaacaggcgtccctttccctctggataacaacaaaagcaagccgggaggctggctgcctctcctcctgctgtctctgctggtggccacatgggtgctggtggcagggatctatctaatgtggaggcacgaaaggatcaagaagacttccttttctaccaccacactactgccccccattaaggttcttgtggtttacccatctgaaatatgtttccatcacacaatttgttacttcactgaatttcttcaaaaccattgcagaagtgaggtcatccttgaaaagtggcagaaaaagaaaatagcagagatgggtccagtgcagtggcttgccactcaaaagaaggcagcagacaaagtcgtcttccttctttccaatgacgtcaacagtgtgtgcgatggtacctgtggcaagagcgagggcagtcccagtgagaactctcaagacctcttcccccttgcctttaaccttttctgcagtgatctaagaagccagattcatctgcacaaatacgtggtggtctactttagagagattgatacaaaagacgattacaatgctctcagtgtctgccccaagtaccacctcatgaaggatgccactgctttctgtgcagaacttctccatgtcaagcagcaggtgtcagcaggaaaaagatcacaagcctgccacgatggctgctgctccttgtagcccacccatgagaagcaagagaccttaaaggcttcctatcccaccaattacagggaaaaaacgtgtgatgatcctgaagcttactatgcagcctacaaacagccttagtaattaaaacattttataccaataaaattttcaaatattgctaactaatgtagcattaactaacgattggaaactacatttacaacttcaaagctgttttatacatagaaatcaattacagttttaattgaaaactataaccattttgataatgcaacaataaagcatcttcagccaaacatctagtcttccatagaccatgcattgcagtgtacccagaactgtttagctaatattctatgtttaattaatgaatactaactctaagaacccctcactgattcactcaatagcatcttaagtgaaaaaccttctattacatgcaaaaaatcattgtttttaagataacaaaagtagggaataaacaagctgaacccacttttaaaaaaaaaaaaaaaaaaaaaaaaaaaaaaaaa

SEQ ID NO: 150 - protein, 502 aa, homo sapiens
MSLVLLSLAALCRSAVPREPTVQCGSETGPSPEWMLQHDLIPGDLRDLRVEPVTTSVATGDYSILMNVSWVLRADASIRLLKATKICVTGKSNFQSYSCVRCNYTEAFQTQTRPSGGKWTFSYIGFPVELNTVYFIGAHNIPNANMNEDGPSMSVNFTSPGCLDHIMKYKKKCVKAGSLWDPNITACKKNEETVEVNFTTTPLGNRYMALIQHSTIIGFSQVFEPHQKKQTRASVVIPVTGDSEGATVQLTPYFPTCGSDCIRHKGTVVLCPQTGVPFPLDNNKSKPGGWLPLLLLSLLVATWVLVAGIYLMWRHERIKKTSFSTTTLLPPIKVLVVYPSEICFHHTICYFTEFLQNHCRSEVILEKWQKKKIAEMGPVQWLATQKKAADKVVFLLSNDVNSVCDGTCGKSEGSPSENSQDLFPLAFNLFCSDLRSQIHLHKYVVVYFREIDTKDDYNALSVCPKYHLMKDATAFCAELLHVKQQVSAGKRSQACHDGCCSL

SEQ ID NO: 151 - DNA, 772 bp, homo sapiens
ggataacccgcggccgcgcctgcccgctcgcacccctctcccgcgcccggttctccctcgcagcacctcgaagtgcgcccctcgccctcctgctcgcgccccgccgccatggctgcctcccccgcgcggcctgctgtcctggccctgaccgggctggcgctgctcctgctcctgtgctggggcccaggtggcataagtggaaataaactcaagctgatgcttcaaaaacgagaagcacctgttccaactaagactaaagtggccgttgatgagaataaagccaaagaattccttggcagcctgaagcgccagaagcggcagctgtgggaccggactcggcccgaggtgcagcagtggtaccagcagtttctctacatgggctttgacgaagcgaaatttgaagatgacatcacctattggcttaacagagatcgaaatggacatgaatactatggcgattactaccaacgtcactatgatgaagactctgcaattggtccccggagcccctacggctttaggcatggagccagcgtcaactacgatgactactaaccatgacttgccacacgctgtacaagaagcaaatagcgattctcttcatgtatctcctaatgccttacactacttggtttctgatttgctctatttcagcagatctttctacctactttggtgatcaaaaaagaagagttaaaacaacacatgtaaatgccttttgatatttcatgggaatgtttaaaaatagaaataaagcattttgttaaaacga

SEQ ID NO: 152 - protein, 148 aa, homo sapiens
MAASPARPAVLALTGLALLLLLCWGPGGISGNKLKLMLQKREAPVPTKTKVAVDENKAKEFLGSLKRQKRQLWDRTRPEVQQWYQQFLYMGFDEAKFEDDITYWLNRDRNGHEYYGDYYQRHYDEDSAIGPRSPYGFRHGASVNYDDY

SEQ ID NO: 153 - DNA, 2526 bp, homo sapiens
gcacacagtgcacgaagacgctgtcgggagagcccaggattcaacacgggccttgagaaatgtggctcttgtacctcctggtgccggccctgttctgcagggcaggaggctccattcccatccctcagaagttatttggggaggtgacttcccctctgttccccaagccttaccccaacaactttgaaacaaccactgtgatcacagtccccacgggatacagggtgaagctcgtcttccagcagtttgacctggagccttctgaaggctgcttctatgattatgtcaagatctctgctgataagaaaagcctggggaggttctgtgggcaactgggttctccactgggcaaccccccgggaaagaaggaatttatgtcccaagggaacaagatgctgctgaccttccacacagacttctccaacgaggagaatgggaccatcatgttctacaagggcttcctggcctactaccaagctgtggaccttgatgaatgtgcttcccggagcaaattaggggaggaggatccccagccccagtgccagcacctgtgtcacaactacgttggaggctacttctgttcctgccgtccaggctatgagcttcaggaagacaggcattcctgccaggctgagtgcagcagcgagctgtacacggaggcatcaggctacatctccagcctggagtaccctcggtcctacccccctgacctgcgctgcaactacagcatccgggtggagcggggcctcaccctgcacctcaagttcctggagccttttgatattgatgaccaccagcaagtacactgcccctatgaccagctacagatctatgccaacgggaagaacattggcgagttctgtgggaagcaaaggccccccgacctcgacaccagcagcaatgctgtggatctgctgttcttcacagatgagtcgggggacagccggggctggaagctgcgctacaccaccgagatcatcaagtgcccccagcccaagaccctagacgagttcaccatcatccagaacctgcagcctcagtaccagttccgtgactacttcattgctacctgcaagcaaggctaccagctcatagaggggaaccaggtgctgcattccttcacagctgtctgccaggatgatggcacgtggcatcgtgccatgcccagatgcaagatcaaggactgtgggcagccccgaaacctgcctaatggtgacttccgttacaccaccacaatgggagtgaacacctacaaggcccgtatccagtactactgccatgagccatattacaagatgcagaccagagctggcagcagggagtctgagcaaggggtgtacacctgcacagcacagggcatttggaagaatgaacagaagggagagaagattcctcggtgcttgccagtgtgtgggaagcccgtgaaccccgtggaacagaggcagcgcatcatcggagggcaaaaagccaagatgggcaacttcccctggcaggtgttcaccaacatccacgggcgcgggggcggggccctgctgggcgaccgctggatcctcacagctgcccacaccctgtatcccaaggaacacgaagcgcaaagcaacgcctctttggatgtgttcctgggccacacaaatgtggaagagctcatgaagctaggaaatcaccccatccgcagggtcagcgtccacccggactaccgtcaggatgagtcctacaattttgagggggacatcgccctgctggagctggaaaatagtgtcaccctgggtcccaacctcctccccatctgcctccctgacaacgataccttctacgacctgggcttgatgggctatgtcagtggcttcggggtcatggaggagaagattgctcatgacctcaggtttgtccgtctgcccgtagctaatccacaggcctgtgagaactggctccggggaaagaataggatggatgtgttctctcaaaacatgttctgtgctggacacccatctctaaagcaggacgcctgccagggggatagtgggggcgtttttgcagtaagggacccgaacactgatcgctgggtggccacgggcatcgtgtcctggggcatcgggtgcagcaggggctatggcttctacaccaaagtgctcaactacgtggactggatcaagaaagagatggaggaggaggactgagcccagaattcactaggttcgaatccagagagcagtgtggaaaaaaaaaaacaaaaaacaactgaccagttgttgataaccactaagagtctctattaaaattactgatgcagaaagaccgtgtgtgaaattctctttcctgtagtcccattgatgtactttacctgaaacaacccaaagggcccctttctttcttctgaggattgcagaggatatagttatcaatctctagttgtcactttcctcttccactttgataccattgggtcattgaatataactttttccaaataaagttttatgagaaatgccagtgtgcaaaaaaaaaaaaaaaaaaaaaaaaaaaaaa

SEQ ID NO: 154 - protein, 705 aa, homo sapiens
MWLLYLLVPALFCRAGGSIPIPQKLFGEVTSPLFPKPYPNNFETTTVITVPTGYRVKLVFQQFDLEPSEGCFYDYVKISADKKSLGRFCGQLGSPLGNPPGKKEFMSQGNKMLLTFHTDFSNEENGTIMFYKGFLAYYQAVDLDECASRSKLGEEDPQPQCQHLCHNYVGGYFCSCRPGYELQEDRHSCQAECSSELYTEASGYISSLEYPRSYPPDLRCNYSIRVERGLTLHLKFLEPFDIDDHQQVHCPYDQLQIYANGKNIGEFCGKQRPPDLDTSSNAVDLLFFTDESGDSRGWKLRYTTEIIKCPQPKTLDEFTIIQNLQPQYQFRDYFIATCKQGYQLIEGNQVLHSFTAVCQDDGTWHRAMPRCKIKDCGQPRNLPNGDFRYTTTMGVNTYKARIQYYCHEPYYKMQTRAGSRESEQGVYTCTAQGIWKNEQKGEKIPRCLPVCGKPVNPVEQRQRIIGGQKAKMGNFPWQVFTNIHGRGGGALLGDRWILTAAHTLYPKEHEAQSNASLDVFLGHTNVEELMKLGNHPIRRVSVHPDYRQDESYNFEGDIALLELENSVTLGPNLLPICLPDNDTFYDLGLMGYVSGFGVMEEKIAHDLRFVRLPVANPQACENWLRGKNRMDVFSQNMFCAGHPSLKQDACQGDSGGVFAVRDPNTDRWVATGIVSWGIGCSRGYGFYTKVLNYVDWIKKEMEEED

SEQ ID NO: 155 - DNA, 827 bp, homo sapiens
ggaacagtgtggcccgccatgttcttctccgcggcgctccgggcccgggcggctggcctcaccgcccactggggaagacatgtaaggaatttgcataagacagctatgcaaaatggagctggaggagctttatttgtgcacagagatactcctgagaataaccctgatactccatttgatttcacaccagaaaactataagaggatagaggcaattgtaaaaaactatccagaaggccataaagcagcagctgttcttccagtcctggatttagcccaaaggcagaatgggtggttgcccatctctgctatgaacaaggttgcagaagttttacaagtacctccaatgagagtatatgaagtagcaactttttatacaatgtataatcgaaagccagttggaaagtatcacattcaggtctgcactactacaccctgcatgcttcgaaactctgacagcatactggaggccattcagaaaaagcttggaataaaggttggggagactacacctgacaaacttttcactcttatagaagtggaatgtttaggggcctgtgtgaacgcaccaatggttcaaataaatgacaattactatgaggatttgacagctaaggatattgaagaaattattgatgagctcaaggctggcaaaatcccaaaaccagggccaaggagtggacgcttctcttgtgagccagctggaggtcttacctctttgactgaaccacccaagggacctggatttggtgtacaagcaggcctttaatttatattgaactgtaaatatgtcactagagaaataaaatatggacttccaatctacgt

SEQ ID NO: 156 - protein, 249 aa, homo sapiens
MFFSAALRARAAGLTAHWGRHVRNLHKTAMQNGAGGALFVHRDTPENNPDTPFDFTPENYKRIEAIVKNYPEGHKAAAVLPVLDLAQRQNGWLPISAMNKVAEVLQVPPMRVYEVATFYTMYNRKPVGKYHIQVCTTTPCMLRNSDSILEAIQKKLGIKVGETTPDKLFTLIEVECLGACVNAPMVQINDNYYEDLTAKDIEEIIDELKAGKIPKPGPRSGRFSCEPAGGLTSLTEPPKGPGFGVQAGL

SEQ ID NO: 157 - DNA, 2020 bp, homo sapiens
ggcggggcggggctgagcgtgtttacatccgccgggtgcgcggcttcgccgcccgaggtcgttcggctcgggtaccatcctccgcgccatggacaccagcgacctgttcgccagctgcaggaagggggatgtgggccgagtgcgaccatgaacccgtcctgacccaggctctgacctccttcctgcccaggtacctgctggagcagcgagacgtggaggtactatgcctgcttgtgtgggcacgaggagctggtactctaccttctggccaatggagcccgctgcgaggccaacaccttcgatggtgagcgctgcctctatggggcactgagtgaccccatccgccgggctctacgcgattacaagcaggtcacggcttcctgcaggaggcgggattactatgacgacttcttgcagcggcttctagagcagggcatccacagtgacgtggtctttgtagtacacgggaagccattccgggtgcatcgctgcgtcctgggtgcacgtagtgcctactttgccaacatgctggacaccaaatggaagggcaagagtgtcgtggttctcaggcacccactgatcaaccccgtggcctttggggccctgctgcagtacctgtacacaggccgcctggacattggcgtagagcatgtgagtgactgtgagcgcctggccaagcaatgccagctgtgggacctgctcagcgacctggaggccaagtgcgagaaggtgtctgagtttgtggcgtctaagccaggcacgtgtgtgaaggtgctgaccatcgagcccccacctgcagacccccgcctccgggaggacatggcgctgctggccgattgtgccctgccccccgagctccgaggtgatctttgggagctgcccttcccttgtcctgacggcttcaacagctgccctgacatctgcttccgagtggctggctgcagcttcctctgccacaaggcctttttctgtggccgcagtgactacttccgagccctgctggatgaccacttccgagagagcgaggagccagcgacctcagggggccccccagccgtcaccctgcatggcatctcacccgacgtcttcactcacgtgctctactacatgtacagcgaccacactgagctgtcccccgaggcagcctatgatgtgctgagcgtcgccgacatgtacctgctgccaggcctgaagaggctgtgcggccgcagcctggctcagatgctagacgaggacactgtggtgggtgtgtggcgcgtggccaagctcttccgcctggcgcggcttgaggaccagtgcactgagtacatggccaaggtcattgagaagctggtggagcgggaggacttcgtggaggcggtgaaggaggaggcagcggctgtggcagcccggcaggagacggactctatcccgctggtggacgacatccgcttccacgtggccagcacggtgcagacctacagcgccatagaggaggcgcagcagcgtctgcgggcactcgaggacctgctcgtgtccatcggtctggactgttgagcccctggctgggcagccccaggggccaggagctctcttggagacaagcatgtgtatgcgtttgtgtgcagctcttcttcctgctccctgcacattgagggcttcatggggggtgcgaggggctcagtggggcttctcttccctccatgagcctggagaccccaggggaggatccatttgggatgagccccctccccccaatgcacaagccagcccccaagaccctgggggtggacaccactcagggaaacctggggtgggggtgggctttggtcttagcactttccttctccagatcccccctacccaccccagtcccaaatccagtcctctggcccttgcctagccctgaattgcttctctaagctggtgttcccatgcacagggccattcaggaagggctgggggagtgtgtgtggcaataaagcttgaaggcaccgtgggaaaaaaaaaaaaaaaaaaaaaaaaaaaaaaaaaaaaaaaa

SEQ ID NO: 158 - protein, 336 aa, homo sapiens
MLDTKWKGKSVVVLRHPLINPVAFGALLQYLYTGRLDIGVEHVSDCERLAKQCQLWDLLSDLEAKCEKVSEFVASKPGTCVKVLTIEPPPADPRLREDMALLADCALPPELRGDLWELPFPCPDGFNSCPDICFRVAGCSFLCHKAFFCGRSDYFRALLDDHFRESEEPATSGGPPAVTLHGISPDVFTHVLYYMYSDHTELSPEAAYDVLSVADMYLLPGLKRLCGRSLAQMLDEDTVVGVWRVAKLFRLARLEDQCTEYMAKVIEKLVEREDFVEAVKEEAAAVAARQETDSIPLVDDIRFHVASTVQTYSAIEEAQQRLRALEDLLVSIGLDC

SEQ ID NO: 159 - DNA, 2527 bp, homo sapiens
aaaacgaaagcactccgtgctggaagtaggaggagagtcaggactcccaggacagagagtgcacaaactacccagcacagccccctccgccccctctggaggctgaagagggattccagcccctgccacccacagacacgggctgactggggtgtctgccccccttgggggggggcagcacagggcctcaggcctgggtgccacctggcacctagaagatgcctgtgccctggttcttgctgtccttggcactgggccgaagcccagtggtcctttctctggagaggcttgtggggcctcaggacgctacccactgctctccgggcctctcctgccgcctctgggacagtgacatactctgcctgcctggggacatcgtgcctgctccgggccccgtgctggcgcctacgcacctgcagacagagctggtgctgaggtgccagaaggagaccgactgtgacctctgtctgcgtgtggctgtccacttggccgtgcatgggcactgggaagagcctgaagatgaggaaaagtttggaggagcagctgactcaggggtggaggagcctaggaatgcctctctccaggcccaagtcgtgctctccttccaggcctaccctactgcccgctgcgtcctgctggaggtgcaagtgcctgctgcccttgtgcagtttggtcagtctgtgggctctgtggtatatgactgcttcgaggctgccctagggagtgaggtacgaatctggtcctatactcagcccaggtacgagaaggaactcaaccacacacagcagctgcctgccctgccctggctcaacgtgtcagcagatggtgacaacgtgcatctggttctgaatgtctctgaggagcagcacttcggcctctccctgtactggaatcaggtccagggccccccaaaaccccggtggcacaaaaacctgactggaccgcagatcattaccttgaaccacacagacctggttccctgcctctgtattcaggtgtggcctctggaacctgactccgttaggacgaacatctgccccttcagggaggacccccgcgcacaccagaacctctggcaagccgcccgactgcgactgctgaccctgcagagctggctgctggacgcaccgtgctcgctgcccgcagaagcggcactgtgctggcgggctccgggtggggacccctgccagccactggtcccaccgctttcctgggagaacgtcactgtggacaaggttctcgagttcccattgctgaaaggccaccctaacctctgtgttcaggtgaacagctcggagaagctgcagctgcaggagtgcttgtgggctgactccctggggcctctcaaagacgatgtgctactgttggagacacgaggcccccaggacaacagatccctctgtgccttggaacccagtggctgtacttcactacccagcaaagcctccacgagggcagctcgccttggagagtacttactacaagacctgcagtcaggccagtgtctgcagctatgggacgatgacttgggagcgctatgggcctgccccatggacaaatacatccacaagcgctgggccctcgtgtggctggcctgcctactctttgccgctgcgctttccctcatcctccttctcaaaaaggatcacgcgaaagggtggctgaggctcttgaaacaggacgtccgctcggggggtgagtgggagcaagcgctgggcggagggccgcccccggggagccaggcctgtgccagctcacctcttccctccccatctgttttctccggcagcggccgccaggggccgcgcggctctgctcctctactcagccgatgactcgggtttcgagcgcctggtgggcgccctggcgtcggccctgtgccagctgccgctgcgcgtggccgtagacctgtggagccgtcgtgaactgagcgcgcaggggcccgtggcttggtttcacgcgcagcggcgccagaccctgcaggagggcggcgtggtggtcttgctcttctctcccggtgcggtggcgctgtgcagcgagtggctacaggatggggtgtccgggcccggggcgcacggcccgcacgacgccttccgcgcctcgctcagctgcgtgctgcccgacttcttgcagggccgggcgcccggcagctacgtgggggcctgcttcgacaggctgctccacccggacgccgtacccgcccttttccgcaccgtgcccgtcttcacactgccctcccaactgccagacttcctgggggccctgcagcagcctcgcgccccgcgttccgggcggctccaagagagagcggagcaagtgtcccgggcccttcagccagccctggatagctacttccatcccccggggactcccgcgccgggacgcggggtgggaccaggggcgggacctggggcgggggacgggacttaaataaaggcagacgctgtttttctacccatgtggcccaaaaaaaaaaaaaaaaaaaaaaaaaaaaaaaaaaaaaaaaaaaaaaaaaaaaaaaaaaaaa

SEQ ID NO: 160 - protein, 538 aa, homo sapiens
MPVPWFLLSLALGRSPVVLSLERLVGPQDATHCSPGLSCRLWDSDILCLPGDIVPAPGPVLAPTHLQTELVLRCQKETDCDLCLRVAVHLAVHGHWEEPEDEEKFGGAADSGVEEPRNASLQAQVVLSFQAYPTARCVLLEVQVPAALVQFGQSVGSVVYDCFEAALGSEVRIWSYTQPRYEKELNHTQQLPALPWLNVSADGDNVHLVLNVSEEQHFGLSLYWNQVQGPPKPRWHKNLTGPQIITLNHTDLVPCLCIQVWPLEPDSVRTNICPFREDPRAHQNLWQAARLRLLTLQSWLLDAPCSLPAEAALCWRAPGGDPCQPLVPPLSWENVTVDKVLEFPLLKGHPNLCVQVNSSEKLQLQECLWADSLGPLKDDVLLLETRGPQDNRSLCALEPSGCTSLPSKASTRAARLGEYLLQDLQSGQCLQLWDDDLGALWACPMDKYIHKRWALVWLACLLFAAALSLILLLKKDHAKGWLRLLKQDVRSGGEWEQALGGGPPPGSQACASSPLPSPSVFSGSGRQGPRGSAPLLSR

SEQ ID NO: 161 - DNA, 2471 bp, homo sapiens
agaaagcgagcagccacccagctccccgccaccgccatggtccccgacaccgcctgcgttcttctgctcaccctggctgccctcggcgcgtccggacagggccagagcccgttgggctcagacctgggcccgcagatgcttcgggaactgcaggaaaccaacgcggcgctgcaggacgtgcgggagctgctgcggcagcaggtcagggagatcacgttcctgaaaaacacggtgatggagtgtgacgcgtgcgggatgcagcagtcagtacgcaccggcctacccagcgtgcggcccctgctccactgcgcgcccggcttctgcttccccggcgtggcctgcatccagacggagagcggcgcgcgctgcggcccctgccccgcgggcttcacgggcaacggctcgcactgcaccgacgtcaacgagtgcaacgcccacccctgcttcccccgagtccgctgtatcaacaccagcccggggttccgctgcgaggcttgcccgccggggtacagcggccccacccaccagggcgtggggctggctttcgccaaggccaacaagcaggtttgcacggacatcaacgagtgtgagaccgggcaacataactgcgtccccaactccgtgtgcatcaacacccggggctccttccagtgcggcccgtgccagcccggcttcgtgggcgaccaggcgtccggctgccagcggcgcgcacagcgcttctgccccgacggctcgcccagcgagtgccacgagcatgcagactgcgtcctagagcgcgatggctcgcggtcgtgcgtgtgtgccgttggctgggccggcaacgggatcctctgtggtcgcgacactgacctagacggcttcccggacgagaagctgcgctgcccggagcgccagtgccgtaaggacaactgcgtgactgtgcccaactcagggcaggaggatgtggaccgcgatggcatcggagacgcctgcgatccggatgccgacggggacggggtccccaatgaaaaggacaactgcccgctggtgcggaacccagaccagcgcaacacggacgaggacaagtggggcgatgcgtgcgacaactgccggtcccagaagaacgacgaccaaaaggacacagaccaggacggccggggcgatgcgtgcgacgacgacatcgacggcgaccggatccgcaaccaggccgacaactgccctagggtacccaactcagaccagaaggacagtgatggcgatggtataggggatgcctgtgacaactgtccccagaagagcaacccggatcaggcggatgtggaccacgactttgtgggagatgcttgtgacagcgatcaagaccaggatggagacggacatcaggactctcgggacaactgtcccacggtgcctaacagtgcccaggaggactcagaccacgatggccagggtgatgcctgcgacgacgacgacgacaatgacggagtccctgacagtcgggacaactgccgcctggtgcctaaccccggccaggaggacgcggacagggacggcgtgggcgacgtgtgccaggacgactttgatgcagacaaggtggtagacaagatcgacgtgtgtccggagaacgctgaagtcacgctcaccgacttcagggccttccagacagtcgtgctggacccggagggtgacgcgcagattgaccccaactgggtggtgctcaaccagggaagggagatcgtgcagacaatgaacagcgacccaggcctggctgtgggttacactgccttcaatggcgtggacttcgagggcacgttccatgtgaacacggtcacggatgacgactatgcgggcttcatctttggctaccaggacagctccagcttctacgtggtcatgtggaagcagatggagcaaacgtattggcaggcgaaccccttccgtgctgtggccgagcctggcatccaactcaaggctgtgaagtcttccacaggccccggggaacagctgcggaacgctctgtggcatacaggagacacagagtcccaggtgcggctgctgtggaaggacccgcgaaacgtgggttggaaggacaagaagtcctatcgttggttcctgcagcaccggccccaagtgggctacatcagggtgcgattctatgagggccctgagctggtggccgacagcaacgtggtcttggacacaaccatgcggggtggccgcctgggggtcttctgcttctcccaggagaacatcatctgggccaacctgcgttaccgctgcaatgacaccatcccagaggactatgagacccatcagctgcggcaagcctagggaccagggtgaggacccgccggatgacagccaccctcaccgcggctggatgggggctctgcacccagccccaaggggtggccgtcctgagggggaagtgagaagggctcagagaggacaaaataaagtgtgtgtgcagggaaaaaaaaaaaaaaaaaaaa

SEQ ID NO: 162 - protein, 757 aa, homo sapiens
MVPDTACVLLLTLAALGASGQGQSPLGSDLGPQMLRELQETNAALQDVRELLRQQVREITFLKNTVMECDACGMQQSVRTGLPSVRPLLHCAPGFCFPGVACIQTESGARCGPCPAGFTGNGSHCTDVNECNAHPCFPRVRCINTSPGFRCEACPPGYSGPTHQGVGLAFAKANKQVCTDINECETGQHNCVPNSVCINTRGSFQCGPCQPGFVGDQASGCQRRAQRFCPDGSPSECHEHADCVLERDGSRSCVCAVGWAGNGILCGRDTDLDGFPDEKLRCPERQCRKDNCVTVPNSGQEDVDRDGIGDACDPDADGDGVPNEKDNCPLVRNPDQRNTDEDKWGDACDNCRSQKNDDQKDTDQDGRGDACDDDIDGDRIRNQADNCPRVPNSDQKDSDGDGIGDACDNCPQKSNPDQADVDHDFVGDACDSDQDQDGDGHQDSRDNCPTVPNSAQEDSDHDGQGDACDDDDDNDGVPDSRDNCRLVPNPGQEDADRDGVGDVCQDDFDADKVVDKIDVCPENAEVTLTDFRAFQTVVLDPEGDAQIDPNWVVLNQGREIVQTMNSDPGLAVGYTAFNGVDFEGTFHVNTVTDDDYAGFIFGYQDSSSFYVVMWKQMEQTYWQANPFRAVAEPGIQLKAVKSSTGPGEQLRNALWHTGDTESQVRLLWKDPRNVGWKDKKSYRWFLQHRPQVGYIRVRFYEGPELVADSNVVLDTTMRGGRLGVFCFSQENIIWANLRYRCNDTIPEDYETHQLRQA

SEQ ID NO: 163-166 - skipped (4 entries)

SEQ ID NO: 167 - DNA, 1982 bp, homo sapiens
ggcgagaggcgggctgaggcggcccagcggcggcaggtgaggcggaaccaaccctcctggccatgggaggggccgtggtggacgagggccccacaggcgtcaaggcccctgacggcggctggggctgggccgtgctcttcggctgtttcgtcatcactggcttctcctacgccttccccaaggccgtcagtgtcttcttcaaggagctcatacaggagtttgggatcggctacagcgacacagcctggatctcctccatcctgctggccatgctctacgggacaggtccgctctgcagtgtgtgcgtgaaccgctttggctgccggcccgtcatgcttgtggggggtctctttgcgtcgctgggcatggtggctgcgtccttttgccggagcatcatccaggtctacctcaccactggggtcatcacggggttgggtttggcactcaacttccagccctcgctcatcatgctgaaccgctacttcagcaagcggcgccccatggccaacgggctggcggcagcaggtagccctgtcttcctgtgtgccctgagcccgctggggcagctgctgcaggaccgctacggctggcggggcggcttcctcatcctgggcggcctgctgctcaactgctgcgtgtgtgccgcactcatgaggcccctggtggtcacggcccagccgggctcggggccgccgcgaccctcccggcgcctgctagacctgagcgtcttccgggaccgcggctttgtgctttacgccgtggccgcctcggtcatggtgctggggctcttcgtcccgcccgtgttcgtggtgagctacgccaaggacctgggcgtgcccgacaccaaggccgccttcctgctcaccatcctgggcttcattgacatcttcgcgcggccggccgcgggcttcgtggcggggcttgggaaggtgcggccctactccgtctacctcttcagcttctccatgttcttcaacggcctcgcggacctggcgggctctacggcgggcgactacggcggcctcgtggtcttctgcatcttctttggcatctcctacggcatggtgggggccctgcagttcgaggtgctcatggccatcgtgggcacccacaagttctccagtgccattggcctggtgctgctgatggaggcggtggccgtgctcgtcgggcccccttcgggaggcaaactcctggatgcgacccacgtctacatgtacgtgttcatcctggcgggggccgaggtgctcacctcctccctgattttgctgctgggcaacttcttctgcattaggaagaagcccaaagagccacagcctgaggtggcggccgcggaggaggagaagctccacaagcctcctgcagactcgggggtggacttgcgggaggtggagcatttcctgaaggctgagcctgagaaaaacggggaggtggttcacaccccggaaacaagtgtctgagtggctgggcggggccggcaggcacagggaggaggtacagaagccggcaacgcttgctatttattttacaaactggactggctcaggcagggccacggctgggctccagctgccggcccagcggatcgtcgcccgatcagtgttttgagggggaaggtggcggggtgggaaccgtgtcattccagagtggatctgcggtgaagccaagccgcaaggttacaaggcatcctcaccaggggccccgcctgctgctcccaggtggcctgcggccactgctatgctcaaggacctggaaacccatgcttcgagacaacgtgactttaatgggagggtgggtgggccgcagacaggctggcagggcaggtgctgcgtggggccctctccagcccgtcctaccctgggctcacatggggcctgtgcccacccctcttgagtgtcttggggacagctctttccacccctggaagatggaaataaacctgcgtgtgggtggagtgttctcgtgccgaattcaaaaagctt

SEQ ID NO: 168 - protein, 465 aa, homo sapiens
MGGAVVDEGPTGVKAPDGGWGWAVLFGCFVITGFSYAFPKAVSVFFKELIQEFGIGYSDTAWISSILLAMLYGTGPLCSVCVNRFGCRPVMLVGGLFASLGMVAASFCRSIIQVYLTTGVITGLGLALNFQPSLIMLNRYFSKRRPMANGLAAAGSPVFLCALSPLGQLLQDRYGWRGGFLILGGLLLNCCVCAALMRPLVVTAQPGSGPPRPSRRLLDLSVFRDRGFVLYAVAASVMVLGLFVPPVFVVSYAKDLGVPDTKAAFLLTILGFIDIFARPAAGFVAGLGKVRPYSVYLFSFSMFFNGLADLAGSTAGDYGGLVVFCIFFGISYGMVGALQFEVLMAIVGTHKFSSAIGLVLLMEAVAVLVGPPSGGKLLDATHVYMYVFILAGAEVLTSSLILLLGNFFCIRKKPKEPQPEVAAAEEEKLHKPPADSGVDLREVEHFLKAEPEKNGEVVHTPETSV

SEQ ID NO: 169 - DNA, 2357 bp, homo sapiens; identical to SEQ ID NO: 115

SEQ ID NO: 170 - protein, 155 aa, homo sapiens; identical to SEQ ID NO: 116

SEQ ID NO: 171 - DNA, 3213 bp, homo sapiens
agagactcaagatgattccctttttacccatgttttctctactattgctgcttattgttaaccctataaacgccaacaatcattatgacaagatcttggctcatagtcgtatcaggggtcgggaccaaggcccaaatgtctgtgcccttcaacagattttgggcaccaaaaagaaatacttcagcacttgtaagaactggtataaaaagtccatctgtggacagaaaacgactgttttatatgaatgttgccctggttatatgagaatggaaggaatgaaaggctgcccagcagttttgcccattgaccatgtttatggcactctgggcatcgtgggagccaccacaacgcagcgctattctgacgcctcaaaactgagggaggagatcgagggaaagggatccttcacttactttgcaccgagtaatgaggcttgggacaacttggattctgatatccgtagaggtttggagagcaacgtgaatgttgaattactgaatgctttacatagtcacatgattaataagagaatgttgaccaaggacttaaaaaatggcatgattattccttcaatgtataacaatttggggcttttcattaaccattatcctaatggggttgtcactgttaattgtgctcgaatcatccatgggaaccagattgcaacaaatggtgttgtccatgtcattgaccgtgtgcttacacaaattggtacctcaattcaagacttcattgaagcagaagatgacctttcatcttttagagcagctgccatcacatcggacatattggaggcccttggaagagacggtcacttcacactctttgctcccaccaatgaggcttttgagaaacttccacgaggtgtcctagaaaggttcatgggagacaaagtggcttccgaagctcttatgaagtaccacatcttaaatactctccagtgttctgagtctattatgggaggagcagtctttgagacgctggaaggaaatacaattgagataggatgtgacggtgacagtataacagtaaatggaatcaaaatggtgaacaaaaaggatattgtgacaaataatggtgtgatccatttgattgatcaggtcctaattcctgattctgccaaacaagttattgagctggctggaaaacagcaaaccaccttcacggatcttgtggcccaattaggcttggcatctgctctgaggccagatggagaatacactttgctggcacctgtgaataatgcattttctgatgatactctcagcatggttcagcgcctccttaaattaattctgcagaatcacatattgaaagtaaaagttggccttaatgagctttacaacgggcaaatactggaaaccatcggaggcaaacagctcagagtcttcgtatatcgtacagctgtctgcattgaaaattcatgcatggagaaagggagtaagcaagggagaaacggtgcgattcacatattccgcgagatcatcaagccagcagagaaatccctccatgaaaagttaaaacaagataagcgctttagcaccttcctcagcctacttgaagctgcagacttgaaagagctcctgacacaacctggagactggacattatttgtgccaaccaatgatgcttttaagggaatgactagtgaagaaaaagaaattctgatacgggacaaaaatgctcttcaaaacatcattctttatcacctgacaccaggagttttcattggaaaaggatttgaacctggtgttactaacattttaaagaccacacaaggaagcaaaatctttctgaaagaagtaaatgatacacttctggtgaatgaattgaaatcaaaagaatctgacatcatgacaacaaatggtgtaattcatgttgtagataaactcctctatccagcagacacacctgttggaaatgatcaactgctggaaatacttaataaattaatcaaatacatccaaattaagtttgttcgtggtagcaccttcaaagaaatccccgtgactgtctatacaactaaaattataaccaaagttgtggaaccaaaaattaaagtgattgaaggcagtcttcagcctattatcaaaactgaaggacccacactaacaaaagtcaaaattgaaggtgaacctgaattcagactgattaaagaaggtgaaacaataactgaagtgatccatggagagccaattattaaaaaatacaccaaaatcattgatggagtgcctgtggaaataactgaaaaagagacacgagaagaacgaatcattacaggtcctgaaataaaatacactaggatttctactggaggtggagaaacagaagaaactctgaagaaattgttacaagaagaggtcaccaaggtcaccaaattcattgaaggtggtgatggtcatttatttgaagatgaagaaattaaaagactgcttcagggagacacacccgtgaggaagttgcaagccaacaaaaaagttcaaggttctagaagacgattaagggaaggtcgttctcagtgaaaatccaaaaaccagaaaaaaatgtttatacaaccctaagtcaataacctgaccttagaaaattgtgagagccaagttgacttcaggaactgaaacatcagcacaaagaagcaatcatcaaataattctgaacacaaatttaatatttttttttctgaatgagaaacatgagggaaattgtggagttagcctcctgtggtaaaggaattgaagaaaatataacaccttacaccctttttcatcttgacattaaaagttctggctaactttggaatccattagagaaaaatccttgtcaccagattcattacaattcaaatcgaagagttgtgaactgttatcccattgaaaagaccgagccttgtatgtatgttatggatacataaaatgcacgcaagccattatctctccatgggaagctaagttataaaaataggtgcttggtgtacaaaactttttatatcaaaaggctttgcacatttctatatgagtgggtttactggtaaattatgttattttttacaactaattttgtactctcagaatgtttgtcatatgcttcttgcaatgcatattttttaatctcaaacgtttcaataaaaccatttttcagatataaagagaattacttcaaattgagtaattcagaaaaactcaagatttaagttaaaaagtggtttggacttgggaa

SEQ ID NO: 172 - DNA, 2589 bp, homo sapiens
caaccagggagatttctccattttcctcttgtctacagtgcggctacaaatctgggatttttttattacttctttttttttcgaactacacttgggctcctttttttgtgctcgacttttccaccctttttccctccctcctgtgctgctgctttttgatctcttcgactaaaatttttttatccggagtgtatttaatcggttctgttctgtcctctccaccacccccacccccctccctccggtgtgtgtgccgctgccgctgttgccgccgccgctgctgctgctgctcgccccgtcgttacaccaacccgaggctctttgtttcccctcttggatctgttgagtttctttgttgaagaagccagcatgggtgcccagttctccaagaccgcagcgaagggagaagccgccgcggagaggcctggggaggcggctgtggcctcgtcgccttccaaagcgaacggacaggagaatggccacgtgaaggtaaacggcgacgcttcgcccgcggccgccgagtcgggcgccaaggaggagctgcaggccaacggcagcgccccggccgccgacaaggaggagcccgcggccgccgggagcggggcggcgtcgccctcctcggccgagaaaggtgagccggccgccgccgctgcccccgaggccggggccagcccggtagagaaggaggcccccgcggaaggcgaggctgccgagcccggctcggccacggccgcggagggagaggccgcgtcggccgcctcctcgacttcttcgcccaaggccgaggacggggccacgccctcgcccagcaacgagaccccgaaaaaaaaaaagaagcgcttttccttcaagaagtctttcaagctgagcggcttctccttcaagaagaacaagaaggaggctggagaaggcggtgaggctgaggcgcccgctgccgaaggcggcaaggacgaggccgccgggggcgcagctgcggccgccgccgaggcgggcgcggcctccggggagcaggcagcggcgccgggcgaggaggcggcagcgggcgaggagggggcggcgggtggcgacccgcaggaggccaagccccaggaggccgctgtcgcgccagagaagccgcccgccagcgacgagaccaaggccgccgaggagcccagcaaggtggaggagaaaaaggccgaggaggccggggccagcgccgccgcctgcgaggccccctccgccgccgggcccggcgcgcccccggagcaggaggcagcccccgcggaggagcccgcggccgccgcagcctcgtcagcctgcgcagccccctcacaggaggcccagcccgagtgcagtccagaagcccccccagcggaggcggcagagtaaaagagcaagcttttgtgagataatcgaagaacttttctcccccgtttgtttgttggagtggtgccaggtactgttttggagaacttgtctacaaccagggattgattttaaagatgtctttttttattttacttttttttaagcaccaaattttgttgtttttttttttctcccctccccacagatcccatctcaaatcattctgttaaccaccattccaacaggtcgaggagagcttaaacaccttcttcctctgccttgtttctcttttattttttattttttcgcatcagtattaatgtttttgcatactttgcatctttattcaaaagtgtaaactttctttgtcaatctatggacatgcccatatatgaaggagatgggtgggtcaaaaagggatatcaaatgaagtgataggggtcacaatggggaaattgaagtggtgcataacattgccaaaatagtgtgccactagaaatggtgtaaaggctgtctttttttttttttttaaagaaaagttattaccatgtattttgtgaggcaggtttacaacactacaagtcttgagttaagaaggaaagaggaaaaaagaaaaaacaccaatacccagatttaaaaaaaaaaaaacgatcatagtcttaggagttcatttaaaccataggaacttttcacttatctcatgttagctgtaccagtcagtgattaagtagaactacaagttgtataggctttattgtttattgctggtttatgaccttaataaagtgtaattatgtattaccagcagggtgtttttaactgtgactattgtataaaaacaaatcttgatatccagaagcacatgaagtttgcaactttccaccctgcccatttttgtaaaactgcagtcatcttggaccttttaaaacacaaattttaaactcaaccaagctgtgataagtggaatggttactgtttatactgtggtatgtttttgattacagcagataatgctttcttttccagtcgtctttgagaataaaggaaaaaaaatcttcagatgcaatggttttgtgtagcatcttgtctatcatgttttgtaaatactggagaagctttgaccaatttgacttagagatggaatgtaactttgcttacaaaaattgctattaaactcctgcttaaggtgttctaattttctgtgagcacactaaaagcgaaaaataaatgtgaataaaatgt

SEQ ID NO: 173 - DNA, 3119 bp, homo sapiens
gtgacagaagtagtaggaagtgagctgttcagaggcaggagggtctattctttgccaaaggggggaccagaattccccatgcgagctgtttgaggactgggatgccgagaacgcgagcgatccgagcagggtttgtctgggcaccgtcggggtaggatccggaacgcattcggaaggctttttgcaagcatttacttggaaggagaacttgggatctttctgggaaccccccgccccggctggattggccgagcaagcctggaaaatggtaaatgatcatttggatcaattacaggcttttagctggcttgtctgtcataattcatgattcggggctgggaaaaagaccaacagcctacgtgccaaaaaaggggcagagtttgatggagttgggtggacttttctatgccatttgcctccacacctagaggataagcacttttgcagacattcagtgcaagggagatcatgtttgactgtatggatgttctgtcagtgagtcctgggcaaatcctggatttctacactgcgagtccgtcttcctgcatgctccaggagaaagctctcaaagcatgcttcagtggattgacccaaaccgaatggcagcatcggcacactgctcaatcaattgaaacacagagcaccagctctgaggaactcgtcccaagccccccatctccacttcctccccctcgagtgtacaaaccctgcttcgtctgccaggacaaatcatcagggtaccactatggggtcagcgcctgtgagggatgtaagggctttttccgcagaagtattcagaagaatatgatttacacttgtcaccgagataagaactgtgttattaataaagtcaccaggaatcgatgccaatactgtcgactccagaagtgctttgaagtgggaatgtccaaagaatctgtcaggaatgacaggaacaagaaaaagaaggagacttcgaagcaagaatgcacagagagctatgaaatgacagctgagttggacgatctcacagagaagatccgaaaagctcaccaggaaactttcccttcactctgccagctgggtaaatacaccacgaattccagtgctgaccatcgagtccgactggacctgggcctctgggacaaattcagtgaactggccaccaagtgcattattaagatcgtggagtttgctaaacgtctgcctggtttcactggcttgaccatcgcagaccaaattaccctgctgaaggccgcctgcctggacatcctgattcttagaatttgcaccaggtataccccagaacaagacaccatgactttctcagacggccttaccctaaatcgaactcagatgcacaatgctggatttggtcctctgactgaccttgtgttcacctttgccaaccagctcctgcctttggaaatggatgacacagaaacaggccttctcagtgccatctgcttaatctgtggagaccgccaggaccttgaggaaccgacaaaagtagataagctacaagaaccattgctggaagcactaaaaatttatatcagaaaaagacgacccagcaagcctcacatgtttccaaagatcttaatgaaaatcacagatctccgtagcatcagtgctaaaggtgcagagcgtgtaattaccttgaaaatggaaattcctggatcaatgccacctctcattcaagaaatgctggagaattctgaaggacatgaacccttgaccccaagttcaagtgggaacacagcagagcacagtcctagcatctcacccagctcagtggaaaacagtggggtcagtcagtcaccactcgtgcaataagacattttctagctacttcaaacattccccagtaccttcagttccaggatttaaaatgcaagaaaaaacatttttactgctgcttagtttttggactgaaaagatattaaaactcaagaaggaccaagaagttttcatatgtatcaatatatatactcctcactgtgtaacttacctagaaatacaaacttttccaattttaaaaaatcagccatttcatgcaaccagaaactagttaaaagcttctattttcctctttgaacactcaagatgcatggcaaagacccagtcaaaatgatttacccctggttaagtttctgaagactttgtacatacagaagtatggctctgttctttctatactgtatgtttggtgctttccttttgtcttgcatactcaaaataaccatgacaccaaggttatgaaatagactactgtacacgtctacctaggttcaaaaagataactgtcttgctttcatggaatagtcaagacatcaaggtaaggaaacaggactattgacaggactattgtacagtatgacaagataaggctgaagatattctactttagttagtatggaagcttgtctttgctctttctgatgctctcaaactgcatcttttatttcatgttgcccagtaaaagtatacaaattccctgcactagcagaagagaattctgtatcagtgtaactgccagttcagttaatcaaatgtcatttgttcaattgttaatgtcactttaaattaaaagtggtttattacttgtttaatgacataactacacagttagttaaaaaaaatttttttacagtaatgatagcctccaaggcagaaacacttttcagtgttaagtttttgtttacttgttcacaagccattagggaaatttcatgggataattagcaggctggtctaccactggaccatgtaactctagtgtccttcctgattcatgcctgatattgggatttttttccagcccttcttgatgccaagggctaattatattacatcccaaagaaacaggcatagaatctgcctcctttgaccttgttcaatcactatgaagcagagtgaaagctgtggtagagtggttaacagatacaagtgtcagtttcttagttctcatttaagcactactggaatttttttttttgatatattagcaagtctgtgatgtactttcactggctctgtttgtacattgagattgtttgtttaacaatgctttctatgttcatatactgtttacctttttccatggactctcctggcaaagaataaaatatatttatttt

SEQ ID NO: 174 - DNA, 2762 bp, homo sapiens
gtgacagaagtagtaggaagtgagctgttcagaggcaggagggtctattctttgccaaaggggggaccagaattccccatgcgagctgtttgaggactgggatgccgagaacgcgagcgatccgagcagggtttgtctgggcaccgtcggggtaggatccggaacgcattcggaaggctttttgcaagcatttacttggaaggagaacttgggatctttctgggaaccccccgccccggctggattggccgagcaagcctggaaaatgcaattgaaacacagagcaccagctctgaggaactcgtcccaagccccccatctccacttcctccccctcgagtgtacaaaccctgcttcgtctgccaggacaaatcatcagggtaccactatggggtcagcgcctgtgagggatgtaagggctttttccgcagaagtattcagaagaatatgatttacacttgtcaccgagataagaactgtgttattaataaagtcaccaggaatcgatgccaatactgtcgactccagaagtgctttgaagtgggaatgtccaaagaatctgtcaggaatgacaggaacaagaaaaagaaggagacttcgaagcaagaatgcacagagagctatgaaatgacagctgagttggacgatctcacagagaagatccgaaaagctcaccaggaaactttcccttcactctgccagctgggtaaatacaccacgaattccagtgctgaccatcgagtccgactggacctgggcctctgggacaaattcagtgaactggccaccaagtgcattattaagatcgtggagtttgctaaacgtctgcctggtttcactggcttgaccatcgcagaccaaattaccctgctgaaggccgcctgcctggacatcctgattcttagaatttgcaccaggtataccccagaacaagacaccatgactttctcagacggccttaccctaaatcgaactcagatgcacaatgctggatttggtcctctgactgaccttgtgttcacctttgccaaccagctcctgcctttggaaatggatgacacagaaacaggccttctcagtgccatctgcttaatctgtggagaccgccaggaccttgaggaaccgacaaaagtagataagctacaagaaccattgctggaagcactaaaaatttatatcagaaaaagacgacccagcaagcctcacatgtttccaaagatcttaatgaaaatcacagatctccgtagcatcagtgctaaaggtgcagagcgtgtaattaccttgaaaatggaaattcctggatcaatgccacctctcattcaagaaatgctggagaattctgaaggacatgaacccttgaccccaagttcaagtgggaacacagcagagcacagtcctagcatctcacccagctcagtggaaaacagtggggtcagtcagtcaccactcgtgcaataagacattttctagctacttcaaacattccccagtaccttcagttccaggatttaaaatgcaagaaaaaacatttttactgctgcttagtttttggactgaaaagatattaaaactcaagaaggaccaagaagttttcatatgtatcaatatatatactcctcactgtgtaacttacctagaaatacaaacttttccaattttaaaaaatcagccatttcatgcaaccagaaactagttaaaagcttctattttcctctttgaacactcaagatgcatggcaaagacccagtcaaaatgatttacccctggttaagtttctgaagactttgtacatacagaagtatggctctgttctttctatactgtatgtttggtgctttccttttgtcttgcatactcaaaataaccatgacaccaaggttatgaaatagactactgtacacgtctacctaggttcaaaaagataactgtcttgctttcatggaatagtcaagacatcaaggtaaggaaacaggactattgacaggactattgtacagtatgacaagataaggctgaagatattctactttagttagtatggaagcttgtctttgctctttctgatgctctcaaactgcatcttttatttcatgttgcccagtaaaagtatacaaattccctgcactagcagaagagaattctgtatcagtgtaactgccagttcagttaatcaaatgtcatttgttcaattgttaatgtcactttaaattaaaagtggtttattacttgtttaatgacataactacacagttagttaaaaaaaatttttttacagtaatgatagcctccaaggcagaaacacttttcagtgttaagtttttgtttacttgttcacaagccattagggaaatttcatgggataattagcaggctggtctaccactggaccatgtaactctagtgtccttcctgattcatgcctgatattgggatttttttccagcccttcttgatgccaagggctaattatattacatcccaaagaaacaggcatagaatctgcctcctttgaccttgttcaatcactatgaagcagagtgaaagctgtggtagagtggttaacagatacaagtgtcagtttcttagttctcatttaagcactactggaatttttttttttgatatattagcaagtctgtgatgtactttcactggctctgtttgtacattgagattgtttgtttaacaatgctttctatgttcatatactgtttacctttttccatggactctcctggcaaagaataaaatatatttatttt

SEQ ID NO: 175 - DNA, 1820 bp, homo sapiens
gcggcgacgacggcggcggcagcgctccaactggctcctcgctccgggctccgccgtcgagccgggagagagcctccgccagcggccaggcaccagccagacgacgccagcgaccccggcctctcggcggcaccgcgctaactcaggggctgcataggcacccagagccgaactccaagatgggaggcaagctcagcaagaagaagaagggctacaatgtgaacgacgagaaagccaaggagaaagacaagaaggccgagggcgcggcgacggaagaggaggggaccccgaaggagagtgagccccaggcggccgcagagcccgccgaggccaaggagggcaaggagaagcccgaccaggacgccgagggcaaggccgaggagaaggagggcgagaaggacgcggcggctgccaaggaggaggccccgaaggcggagcccgagaagacggagggcgcggcagaggccaaggctgagcccccgaaggcgcccgagcaggagcaggcggcccccggccccgctgcgggcggcgaggcccccaaagctgctgaggccgccgcggccccggccgagagcgcggcccctgccgccggggaggagcccagcaaggaggaaggggaacccaaaaagactgaggcgcccgcagctcctgccgcccaggagaccaaaagtgacggggccccagcttcagactcaaaacccggcagctcggaggctgccccctcttccaaggagacccccgcagccacggaagcgcctagttccacacccaaggcccagggccccgcagcctctgcagaagagcccaagccggtggaggccccggcagctaattccgaccaaaccgtaaccgtgaaagagtgacaaggacagcctataggaaaaacaataccacttaaaacaatctcctctctctctctctctctctctctctatctctctctctatctcctctctctctctcctctcctatctctcctctctctctctcctatactaacttgtttcaaattggaagtaatgatatgtattgcccaaggaaaaatacaggatgttgtcccatcaagggagggagggggtgggagaatccaaatagtatttttgtggggaaatatctaatataccttcagtcaactttaccaagaagtcctggatttccaagatccgcgtctgaaagtgcagtacatcgtttgtacctgaaactgccgccacatgcactcctccaccgctgagagttgaatagcttttcttctgcaatgggagttgggagtgatgcgtttgattctgcccacagggcctgtgccaaggcaatcagatctttatgagagcagtattttctgtgttttctttttaatttacagcctttcttattttgatatttttttaatgttgtggatgaatgccagctttcagacagagcccacttagcttgtccacatggatctcaatgccaatcctccattcttcctctccagatatttttgggagtgacaaacattctctcatcctacttagcctacctagatttctcatgacgagttaatgcatgtccgtggttgggtgcacctgtagttctgtttattggtcagtggaaatgaaaaaaaaaaaaaaaaaaagtctgcgttcattgcagttccagtttctcttccattctgtgtcacagacaccaacacaccactcattggaaaatggaaaaaaaaaacaaaaaaaaaacaaaaaaatgtacaatggatgcattgaaattatatgtaattgtataaatggtgcaacagtaataaagttaaacaattaaaaagaaaaaaaaaaaaaaaaaaaaaaaaa

SEQ ID NO: 176 - DNA, 3076 bp, homo sapiens
ctatcagtctcgcgctcaccgcccggctgaggaggtgaaagtttctccccaggaagataaaccgcaaaagacaatattgtgcatgatttgcgccttttctttggctttttctttctttcttcacccccccacccacttttttttttttttttttcaaaaagcagagagggaaaaacggagagtgaaggagcgaggaggcgagcgtgagagaaaggagagagagagaaaagaaagggcgaggggctagtggagaagtaaggaggggcgcgctgcgcgaggcggagagagggcgaagcagtcgcggcactggcgctcacattcctctatgctacaaatccaggaggaagtttttttttagggggctgagatgctccatgcctttccccgggcagccttgacgcgcggccctctcggcagagactgagcggcgagaaagtgcgagccgggccggcagaatctgcctggcgggcgctggagcctgcgttactcgcggcccgcagccgtccggctactttgcgtttggcccggccagcgcccgggcgcgccgcgccattgcctgcaggctaggacttcgcgaggtgggtcgactccccctccctcctcctcttcttcctcctcttcctcctcctcttgttcctcctcctcctcccgattttccctcctcggctggcgagggtggggggggcgggggaggccggggctcgccccgagcagccacgatgctcctggacgccggcccccagtacccagcgatcggcgtgaccacctttggcgcgtcccgccaccactccgcgggcgacgtggccgaacgagacgtgggcctgggcatcaacccgttcgccgacggcatgggcgccttcaagctcaaccccagttcgcacgagctggcttcggccggccagacggccttcacgtcgcaggcgccaggctacgcggctgctgcggccctgggccatcaccatcacccgggccacgtcggctcctattccagcgcagccttcaactccacgcgggactttctgttccgcaaccggggttttggcgacgcggcggcggcagccagcgcacagcacagcctctttgctgcatcggccgggggcttcgggggcccacacggccacacggacgccgcgggccacctcctcttccccgggcttcacgagcaggctgccggccacgcgtcgcctaacgtggtcaacgggcagatgaggctcggcttctcgggggacatgtacccgcgaccggagcagtacggccaggtgaccagcccgcgttcggagcactatgctgcgccgcagctgcacggctacgggcccatgaacgtgaacatggccgcgcatcacggcgccggcgccttcttccgctacatgcgccaacccatcaagcaagagctcatctgcaagtggatcgagcccgagcagctggccaaccccaaaaagtcgtgcaacaaaactttcagcaccatgcacgagctagttacgcacgtcaccgtggagcacgtaggtggcccggagcagagtaatcacatctgcttctgggaggagtgtccgcgcgagggcaagcccttcaaagccaaatacaaactggttaaccacatccgcgtgcacacgggcgagaagccctttccctgccccttccctggctgtggcaaggtcttcgcgcgctccgagaatttaaagatccacaaaaggacgcacacaggggagaagcccttcaagtgcgagtttgagggctgtgaccggcgcttcgctaacagcagcgaccgcaagaagcacatgcacgtgcacacgagcgacaagccctatctttgcaagatgtgcgacaagtcctacacgcatcccagttcgctgcgcaaacacatgaaggtccacgaatcctcctcgcagggctcgcagccttcgccggccgccagctctggctacgaatcctccacgcctcccaccatcgtgtctccctccacagacaacccgaccacaagctccttatcgccctcctcctccgcagtccaccacacagccggccacagtgcgctctcttccaattttaacgaatggtacgtttaaaatcagaaacaaaacatcgaacaaaaccctatttaagagactgatcacacacgtatacacaacattactgaaagaaccctgcgaatcaaaacaacccccacacagaccccgcaatccttttttaaaaaatctgccaatagacccaggacgagtaagagaggaagcatcaaccttttaaaaatttcctttcgctttcattatttttctttttttggcaaaggcttggtacccaaggtgcggtagggggtcgagggggaggaggccacctgaccaaatgccgccaaccccgagggccagtttcttgtcgaattggtacgggctctctggggcttcggcttctttttttctttgttttcttgtaaatacagaattattagcttaaaactgtactgttgaattctgtaaatagttatatctcggttggagcgggtgggtgggattgtggcgttgtggtctttgcattgggggaggggggagggaccggatgggcggggggagggggagggggaggggtgggcggccgaaagccaactgtttgtactgaatggcaagaatgttctagtaaatgtgtaccaaaatgtgaattactttgtacgattacagtctccacgtcgacctaacccaatattattggtattaatgtgctttttttgtataaagtgcaaacatttcgtcccaaagtctaagtactttagtgcagtaaaatgttgtttcatgtcctgtcaagaattcgtatagtacgagcctggatctgcgtgtcaaactgttccatttgtttatgtaaagtgatattaaaaaagatataaactataactgtccgttacttttggcaaaagatacaaccacataatgtatataattcctagtttccatatttatccgcatgtaaagggccggtttatccatgttacagctcttcaatatttatggctagaagaactcgtatgtacactttagtttccagaactgtttggtaacctttcgtaccttattaaagattcttaaatctcaaa

SEQ ID NO: 177 - DNA, 1415 bp, homo sapiens
gtttgcgggtgcgggcgggaccggatttcgtccgtgggcccgggggcggcgggggccggggagtgaggggccggctgagcccacctcgctgggccctccctggcgccccgccttgggcggcggcgagcgcgcgggccgccatgtcgtacatgctcccgcacctgcacaacggctggcaggtggaccaggccatcctctcggaggaggaccgcgtggtcgtcatccgcttcggccacgactgggatcctacgtgcatgaagatggacgaggtcctgtacagcatcgccgagaaggttaaaaattttgcagttatttatcttgtggatattacagaagtgcctgacttcaacaaaatgtatgagttatacgatccatgtactgtcatgtttttcttcaggaacaagcacatcatgattgacttggggactggcaacaacaacaagattaactgggccatggaggacaagcaggagatggtggacatcatcgagacggtgtaccgcggggcccgcaaaggccgcggcctggtggtgtcccccaaggactactccaccaagtaccgctactgaggcgccctcagtctgcgcggataaatgtcgtggagccctttttgtatggaaacgttttaagctatttaaagcctttggaaaatacaggaagctccagggctggagcacctctgagatggaattgataacatggtcttaactcaccgaaataaacaagcacgtggtgagaggagcaggcctacttgtttgttctcaggaaacttaatgaatagattactgattttcctagtcaaagttaattcttacccttggagtaaaacgaaggtgtttatcctgtgagcctgtgcgttttgcatactgggttggtttgctggggctgcggtgacagcatatgccgcgagctgggctttaacagagatgtgtgctctcacagctttgcaggcgggggtctgagatcagggtgtcgcgggtggggggtcactgctgaggccgtgaggggaatctgctcaggcctgtccctggcttctgggggctgctggtggtattttcagttccttggtgtgtggatacttcgccccatctctgccttcacctgtgtcctccctgtgtgggtgctggtgtccaaaatttccccttttcgtagtgacaccagctgtgttggattggggcccaccctgctccagcatggcctaatcttaactaattacatttgcaaggatcttatgtccacaaaagtcacagtctgaggtgctgggggttaggacttcaatatataaattttgcggttacacaattcaatccatgacagaatccaaaggtttactctggttataaaaacagtacaataaaatattgtttatagccttccctgtaaggaaaaaaaaaaaaaaaaaaaaaaaaaaaaaa

SEQ ID NO: 178 - DNA, 1898 bp, homo sapiens
ggcacgagggtcagctctggttcggagaagcagcggctggcgtgggccatccggggaatgggcgccctcgtgacctagtgttgcggggcaaaaagggtcttgccggcctcgctcgtgcaggggcgtatctgggcgcctgagcgcggcgtgggagccttgggagccgccgcagcagggggcacacccggaaccggcctgagcgcccgggaccatgaacggggaggccatctgcagcgccctgcccaccattccctaccacaaactcgccgacctgcgctacctgagccgcggcgcctctggcactgtgtcgtccgcccgccacgcagactggcgcgtccaggtggccgtgaagcacctgcacatccacactccgctgctcgacagtgaaagaaaggatgtcttaagagaagctgaaattttacacaaagctagatttagttacattcttccaattttgggaatttgcaatgagcctgaatttttgggaatagttactgaatacatgccaaatggatcattaaatgaactcctacataggaaaactgaatatcctgatgttgcttggccattgagatttcgcatcctgcatgaaattgcccttggtgtaaattacctgcacaatatgactcctcctttacttcatcatgacttgaagactcagaatatcttattggacaatgaatttcatgttaagattgcagattttggtttatcaaagtggcgcatgatgtccctctcacagtcacgaagtagcaaatctgcaccagaaggagggacaattatctatatgccacctgaaaactatgaacctggacaaaaatcaagggccagtatcaagcacgatatatatagctatgcagttatcacatgggaagtgttatccagaaaacagccttttgaagatgtcaccaatcctttgcagataatgtatagtgtgtcacaaggacatcgacctgttattaatgaagaaagtttgccatatgatatacctcaccgagcacgtatgatctctctaatagaaagtggatgggcacaaaatccagatgaaagaccatctttcttaaaatgtttaatagaacttgaaccagttttgagaacatttgaagagataacttttcttgaagctgttattcagctaaagaaaacaaagttacagagtgtttcaagtgccattcacctatgtgacaagaagaaaatggaattatctctgaacatacctgtaaatcatggtccacaagaggaatcatgtggatcctctcagctccatgaaaatagtggttctcctgaaacttcaaggtccctgccagctcctcaagacaatgattttttatctagaaaagctcaagactgttattttatgaagctgcatcactgtcctggaaatcacagttgggatagcaccatttctggatctcaaagggctgcattctgtgatcacaagaccactccatgctcttcagcaataataaatccactctcaactgcaggaaactcagaacgtctgcagcctggtatagcccagcagtggatccagagcaaaagggaagacattgtgaaccaaatgacagaagcctgccttaaccagtcgctagatgcccttctgtccagggacttgatcatgaaagaggactatgaacttgttagtaccaagcctacaaggacctcaaaagtcagacaattactagacactactgacatccaaggagaagaatttgccaaagttatagtacaaaaattgaaagataacaaacaaatgggtcttcagccttacccggaaatacttgtggtttctagatcaccatctttaaatttacttcaaaataaaagcatgtaagtgactgtttttcaagaagaaatgtgtttcataaaaggatatttataaaaaaaaaaaaaaaaaa

SEQ ID NO: 179 - DNA, 3949 bp, homo sapiens
gcggtggcgcgggagcggccccggggaccccgcgctgctgacggcggcgaccgcggccggaggcgggcgcgggtctcggaggcggtcgcctcagcaccgccccacgggcggccccagcccctcccgcagccctcctccctcccgctcccttcccgccgcctcctccccgccctcctccctcctcgctcgcgggccgggcccggcatggtgcggcgtcgccgccgatggcgctgaggcggagcatggggcggccggggctcccgccgctgccgctgccgccgccaccgcggctcgggctgctgctggcggctctggcttctctgctgctcccggagtccgccgccgcaggtctgaagctcatgggagccccggtgaagctgacagtgtctcaggggcagccggtgaagctcaactgcagtgtggaggggatggaggagcctgacatccagtgggtgaaggatggggctgtggtccagaacttggaccagttgtacatcccagtcagcgagcagcactggatcggcttcctcagcctgaagtcagtggagcgctctgacgccggccggtactggtgccaggtggaggatgggggtgaaaccgagatctcccagccagtgtggctcacggtagaaggtgtgccatttttcacagtggagccaaaagatctggcagtgccacccaatgcccctttccaactgtcttgtgaggctgtgggtccccctgaacctgttaccattgtctggtggagaggaactacgaagatcgggggacccgctccctctccatctgttttaaatgtaacaggggtgacccagagcaccatgttttcctgtgaagctcacaacctaaaaggcctggcctcttctcgcacagccactgttcaccttcaagcactgcctgcagcccccttcaacatcaccgtgacaaagctttccagcagcaacgctagtgtggcctggatgccaggtgctgatggccgagctctgctacagtcctgtacagttcaggtgacacaggccccaggaggctgggaagtcctggctgttgtggtccctgtgcccccctttacctgcctgctccgggacctggtgcctgccaccaactacagcctcagggtgcgctgtgccaatgccttggggccctctccctatgctgactgggtgccctttcagaccaagggtctagccccagccagcgctccccaaaacctccatgccatccgcacagattcaggcctcatcttggagtgggaagaagtgatccccgaggcccctttggaaggccccctgggaccctacaaactgtcctgggttcaagacaatggaacccaggatgagctgacagtggaggggaccagggccaatttgacaggctgggatccccaaaaggacctgatcgtacgtgtgtgcgtctccaatgcagttggctgtggaccctggagtcagccactggtggtctcttctcatgaccgtgcaggccagcagggccctcctcacagccgcacatcctgggtacctgtggtccttggtgtgctaacggccctggtgacggctgctgccctggccctcatcctgcttcgaaagagacggaaagagacgcggtttgggcaagcctttgacagtgtcatggcccggggagagccagccgttcacttccgggcagcccggtccttcaatcgagaaaggcccgagcgcatcgaggccacattggacagcttgggcatcagcgatgaactaaaggaaaaactggaggatgtgctcatcccagagcagcagttcaccctgggccggatgttgggcaaaggagagtttggttcagtgcgggaggcccagctgaagcaagaggatggctcctttgtgaaagtggctgtgaagatgctgaaagctgacatcattgcctcaagcgacattgaagagttcctcagggaagcagcttgcatgaaggagtttgaccatccacacgtggccaaacttgttggggtaagcctccggagcagggctaaaggccgtctccccatccccatggtcatcttgcccttcatgaagcatggggacctgcatgccttcctgctcgcctcccggattggggagaacccctttaacctacccctccagaccctgatccggttcatggtggacattgcctgcggcatggagtacctgagctctcggaacttcatccaccgagacctggctgctcggaattgcatgctggcagaggacatgacagtgtgtgtggctgacttcggactctcccggaagatctacagtggggactactatcgtcaaggctgtgcctccaaactgcctgtcaagtggctggccctggagagcctggccgacaacctgtatactgtgcagagtgacgtgtgggcgttcggggtgaccatgtgggagatcatgacacgtgggcagacgccatatgctggcatcgaaaacgctgagatttacaactacctcattggcgggaaccgcctgaaacagcctccggagtgtatggaggacgtgtatgatctcatgtaccagtgctggagtgctgaccccaagcagcgcccgagctttacttgtctgcgaatggaactggagaacatcttgggccagctgtctgtgctatctgccagccaggaccccttatacatcaacatcgagagagctgaggagcccactgcgggaggcagcctggagctacctggcagggatcagccctacagtggggctggggatggcagtggcatgggggcagtgggtggcactcccagtgactgtcggtacatactcacccccggagggctggctgagcagccagggcaggcagagcaccagccagagagtcccctcaatgagacacagaggcttttgctgctgcagcaagggctactgccacacagtagctgttagcccacaggcagagggcatcggggccatttggccggctctggtggccactgagctggctgactaagccccgtctgaccccagcccagacagcaaggtgtggaggctcctgtggtagtcctcccaagctgtgctgggaagcccggactgaccaaatcacccaatcccagttcttcctgcaaccactctgtggccagcctggcatcagtttaggccttggcttgatggaagtgggccagtcctggttgtctgaacccaggcagctggcaggagtggggtggttatgtttccatggttaccatgggtgtggatggcagtgtggggagggcaggtccagctctgtgggccctaccctcctgctgagctgcccctgctgcttaagtgcatgcattgagctgcctccagcctggtggcccagctattaccacacttggggtttaaatatccaggtgtgcccctccaagtcacaaagagatgtccttgtaatattcccttttaggtgagggttggtaaggggttggtatctcaggtctgaatcttcaccatctttctgattccgcaccctgcctacgccaggagaagttgaggggagcatgcttccctgcagctgaccgggtcacacaaaggcatgctggagtacccagcctatcaggtgcccctcttccaaaggcagcgtgccgagccagcaagaggaaggggtgctgtgaggcttgcccaggagcaagtgaggccggagaggagttcaggaacccttctccatacccacaatctgagcacgctaccaaatctcaaaatatcctaagactaacaaaggcagctgtgtctgagcccaacccttctaaacggtgacctttagtgccaacttcccctctaactggacagcctcttctgtcccaagtctccagagagaaatcaggcctgatgagggggaattcctggaacctggaccccagccttggtgggggagcctctggaatgcatggggcgggtcctagctgttagggacatttccaagctgttagttgctgtttaaaatagaaataaaattgaagact

SEQ ID NO: 180 - protein, 890 aa, homo sapiens
MALRRSMGRPGLPPLPLPPPPRLGLLLAALASLLLPESAAAGLKLMGAPVKLTVSQGQPVKLNCSVEGMEEPDIQWVKDGAVVQNLDQLYIPVSEQHWIGFLSLKSVERSDAGRYWCQVEDGGETEISQPVWLTVEGVPFFTVEPKDLAVPPNAPFQLSCEAVGPPEPVTIVWWRGTTKIGGPAPSPSVLNVTGVTQSTMFSCEAHNLKGLASSRTATVHLQALPAAPFNITVTKLSSSNASVAWMPGADGRALLQSCTVQVTQAPGGWEVLAVVVPVPPFTCLLRDLVPATNYSLRVRCANALGPSPYADWVPFQTKGLAPASAPQNLHAIRTDSGLILEWEEVIPEAPLEGPLGPYKLSWVQDNGTQDELTVEGTRANLTGWDPQKDLIVRVCVSNAVGCGPWSQPLVVSSHDRAGQQGPPHSRTSWVPVVLGVLTALVTAAALALILLRKRRKETRFGQAFDSVMARGEPAVHFRAARSFNRERPERIEATLDSLGISDELKEKLEDVLIPEQQFTLGRMLGKGEFGSVREAQLKQEDGSFVKVAVKMLKADIIASSDIEEFLREAACMKEFDHPHVAKLVGVSLRSRAKGRLPIPMVILPFMKHGDLHAFLLASRIGENPFNLPLQTLIRFMVDIACGMEYLSSRNFIHRDLAARNCMLAEDMTVCVADFGLSRKIYSGDYYRQGCASKLPVKWLALESLADNLYTVQSDVWAFGVTMWEIMTRGQTPYAGIENAEIYNYLIGGNRLKQPPECMEDVYDLMYQCWSADPKQRPSFTCLRMELENILGQLSVLSASQDPLYINIERAEEPTAGGSLELPGRDQPYSGAGDGSGMGAVGGTPSDCRYILTPGGLAEQPGQAEHQPESPLNETQRLLLLQQGLLPHSSC